(19) United States
(12) Patent Application Publication
(10) Pub. No.: US 2024/0217978 A1
(43) Pub. Date: Jul. 4, 2024

(54) COMPOUND AS ADENOSINE A2a RECEPTOR ANTAGONIST AND PHARMACEUTICAL COMPOSITION COMPRISING SAME

(71) Applicant: CHONG KUN DANG PHARMACEUTICAL CORP., Seoul (KR)

(72) Inventors: Chang Sik Lee, Gyeonggi-do (KR); Jaewon Lee, Gyeonggi-do (KR); Jae Young Lee, Gyeonggi-do (KR); Yesong Park, Gyeonggi-do (KR); Dalyong Gwak, Gyeonggi-do (KR); Hyunjin Michael Kim, Gyeonggi-do (KR)

(21) Appl. No.: 18/287,743

(22) PCT Filed: Apr. 21, 2022
(86) PCT No.: PCT/IB2022/053721

§ 371 Date: Oct. 20, 2023

(30) Foreign Application Priority Data
Apr. 23, 2021 (KR) 10-2021-0053352

(51) Int. Cl. C07D 487/04; A61K 31/519; A61K 31/53; A61K 31/5377; A61K 31/551; C07D 498/04; C07D 513/04; C07D 519/00

(52) U.S. Cl. CPC C07D 487/04; A61K 31/519; A61K 31/53; A61K 31/5377; A61K 31/551; C07D 498/04; C07D 513/04; C07D 519/00

(57) ABSTRACT

The present invention relates to a compound represented by formula 1 as an adenosine A2a receptor antagonist, stereoisomers thereof, pharmaceutically acceptable salts thereof, a method using the same, a medicinal use thereof, and a pharmaceutical composition including the same.

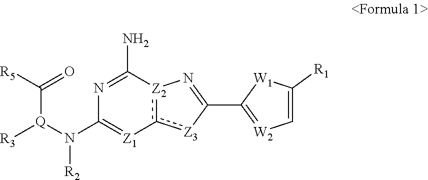

### TECHNICAL FIELD

The present invention relates to a compound as an adenosine A2a receptor antagonist, stereoisomers thereof, pharmaceutically acceptable salts thereof, a medicinal use thereof, and a pharmaceutical composition including the same.

### BACKGROUND ART

Adenosine refers to a variety of biologically active modifiers in the cardiovascular system and the nervous system that regulate various functions through interactions with specific cell surface receptors. In addition, adenosine is an immunosuppressive metabolite produced at a high level in a tumor microenvironment, accumulates in tumors to promote the proliferation of the tumors, and also serves to mediate a tumor escape in the immune system by conferring resistance to the immune system, etc. The tumor microenvironment is one of the important regulators for immune functions that influence cancer progression and metastasis. In the tumor microenvironment, a high concentration of adenosine inhibits the responses of antitumor cytotoxic lymphocytes, and T cells inhibit actions thereof and express an adenosine A2a receptor (A2aR), which blocks the removal of tumors by immunity.

The adenosine A2a receptor is one of A1, A2a, A2b and A3 receptors, which are four subtypes of a G-protein coupled receptor (GPCR) and is widely distributed in human tissues and highly expressed in striatum of the brain, immune cells, spleen, thymus, leukocytes, platelets, GABA-type neurons, olfactory bulbs and the like. At the same time, the adenosine A2a receptor is also expressed in other parts such as the heart, lungs, blood vessels, brain and the like, and exhibits high affinity for adenosine. The A2b receptor is also widely expressed, but mostly at a low level and less sensitive to adenosine. The A2a receptor has been a target of drugs for the treatment of Parkinson's disease and has recently been reported as a promising target for cancer immunotherapy. (J. Med. Chem. 2020, 63, 21, 12196-12212)

Basically, the immune cells of cancer patients develop resistance to cancer antigens and thus can recognize cancer cells, but are functionally inhibited, thus failing to effectively eliminate cancer cells. A key to immunotherapy is to wake up the immune cells that have fallen into resistance and induce them to become activated immune cells to destroy cancer cells. Such immunotherapy includes cytokine therapeutic agents such as interferon gamma, IL-2, etc., cancer vaccines using dendritic cells, cell therapy products using T cells, immune checkpoints of blocking immunosuppressive proteins, and the like.

Immune checkpoint proteins are cell membrane proteins that inhibit the differentiation, proliferation, and activity of immune cells. This suggests that immune checkpoint proteins may be a good target for cancer treatment. Indeed, in several animal cancer models, it has been confirmed that blocking of CTLA4, PD1 and PDL1 with antibodies inhibits cancer growth and increases a survival rate. Such therapeutic effect is based on a mechanism by which an inhibitory signal of the immune checkpoint proteins is blocked and thus cancer-specific T cells are activated. Based on animal test results, many clinical trials have been designed and conducted, and it is known that a much higher therapeutic effect is shown than that of conventional anticancer drugs (Leone and Emens, Journal for ImmunoTherapy of Cancer (2018) 6:57).

It is known that an A2a receptor antagonist may inhibit a key immunosuppressive pathway in the tumor microenvironment of certain cancers. It has been found that adenosine is more highly distributed in the tumor microenvironment of the certain cancers, unlike other normal tissues, and it has been announced that such overexpressed adenosine acts to weaken the core immune system centering on T cells (Cancer Cell, 2015 Apr. 13:27(4): 435-436).

According to a recent study, among the receptors of adenosine, the A2a receptor is particularly known as a major factor in influencing the overexpression of adenosine in the tumor microenvironment of certain cancers, and thus it has been reported that selective and appropriate blocking of this receptor may create a great synergy in anti-PD-1 immunotherapy (Cancer Immunol Res; 3 (5) May 2015; 506-517).

As such, blocking of the adenosine signaling pathway of the A2a receptor may reduce an inhibitory effect on the immune system and enhance the immune functions of T cells, and thus the adenosine A2a receptor antagonist is a promising negative mechanism capable of inhibiting tumor growth.

Accordingly, the present inventors have invented a novel compound structure as an A2a receptor antagonist which selectively inhibits the adenosine A2a receptor, and have used the same to inhibit or treat adenosine A2a receptor-associated diseases, thereby completing the present invention.

### RELATED ART REFERENCES

### Non-Patent Documents

J. Med. Chem. 2020, 63, 21, 12196-12212
Leone and Emens Journal for ImmunoTherapy of Cancer (2018) 6:57
Cancer Cell, 2015 Apr. 13: 27 (4), 435-436
Cancer Immunol Res; 3 (5); 506-517

### DISCLOSURE OF THE INVENTION

### Technical Problem

An object of the present invention is to provide a compound as an A2a receptor antagonist, stereoisomers thereof or pharmaceutically acceptable salts thereof.

Another object of the present invention is to provide a pharmaceutical composition including a compound as an A2a receptor antagonist, stereoisomers thereof or pharmaceutically acceptable salts thereof.

Still another object of the present invention is to provide a composition for treating or preventing adenosine A2a receptor-associated diseases, including a compound as an A2a receptor antagonist, stereoisomers thereof or pharmaceutically acceptable salts thereof.

Still another object of the present invention is to provide a composition for treating or preventing cancer or inflammatory diseases, including a compound as an A2a receptor antagonist, stereoisomers thereof or pharmaceutically acceptable salts thereof.

Still another object of the present invention is to provide a method for treating or preventing adenosine A2a receptor-associated diseases, including administering a therapeutically effective amount of said compound or the pharmaceutical composition including the compound.

Still another object of the present invention is to provide a use for treating or preventing adenosine A2a receptor-associated diseases or a use of said compound for preparing a medicament.

### Technical Solution to Problem

Hereinafter, the present invention will be described in more detail. In other words, all the combinations of various elements disclosed in the present invention fall within the scope of the present invention. In addition, it cannot be seen that the scope of the present invention is limited to the specific description below.

### Compound Represented by Formula 1

According to the objects, the compounds provided in the present invention may be as shown in (1) to (6) below.

The present invention may provide a compound represented by formula 1 below, stereoisomers thereof or pharmaceutically acceptable salts thereof:

(1) A compound represented by formula 1, stereoisomers thereof or pharmaceutically acceptable salts thereof:

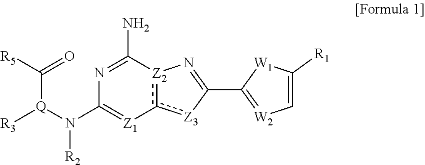

in formula 1,
W₁ is O or S;
W₂ is N or CH;
Z₁ is CH or N;
Z₂ is C or N;
Z₃ is N, O or S;
[symbol] and [symbol] each independently represent a single bond or a double bond (when [symbol] is a bond, [symbol] is a single bond, and when [symbol] is a single bond, [symbol] is a double bond);
Q is C—R₄ or N;
R₁ is H or —CH₃;
R₂ is H or C1-C5 alkyl, R₃ is H or —La-Ra, or R₂ and R₃ are linked to form a ring,
in which La is a single bond or C1-C3 alkylene, Ra is C1-C5 alkyl, C3-C6 cycloalkyl,

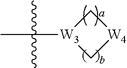

(a and b are each independently 1 or 2, W₃ is CH or N, W₄ is CH₂ or O, in which if W₃ is CH, then W₄ is not CH₂), phenyl or -phenylen-O-benzyl, and if Ra is C1-C5 alkyl or phenyl, then at least one of each H may be substituted with —OH or C1-C5 alkoxy;
a ring formed by linking R₂ and R₃ is a 4- to 6-membered N-containing heterocycloalkyl (in which at least one H of the N-containing heterocycloalkyl may be each independently substituted with C1-C5 alkyl or OH), or a 6- to 8-membered N-containing spiroheterocycloalkyl;
R₄ is H or C1-C5 alkyl;
R₅ is
—NH—(CH₂)_{y}—R_{b} (in which y is any one integer of 1 to 3, and R_{b} is a 5- or 6-membered heterocycloalkyl including any one of O and N);

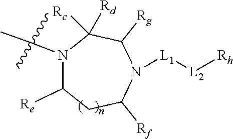

(in which n is 0 or 1, and R_{c}, R_{d}, Rₑ, R_{f} and R_{g} are each independently H or C1-C5 alkyl, but two selected from R_{c}, R_{d}, Rₑ, R_{f} and R_{g} may be linked to form CH₂ or CH₂—CH₂);

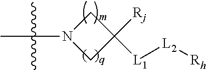

(in which m and q are each independently any one integer of 0 to 3, m and q may not be 0 at the same time, and Rⱼ is H or halogen);

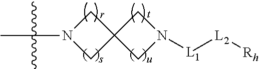

(in which r, s, t and u are each independently 1 or 2);

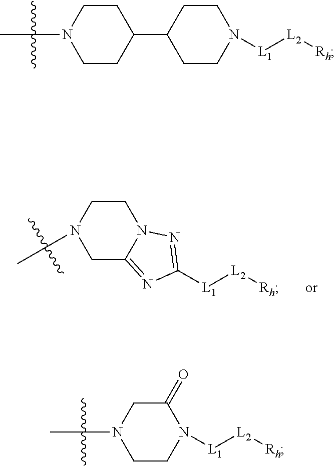

in above R₅,
L₁ is a single bond or C1-C3 alkylene;
L₂ is a single bond, —C(═O)—, —C(═O)NH—, —C(═O)—N(C1-C5 alkyl)-, —C(═O)—NH(C1-C5 alkylene)-, —S(═O)₂— or —S(═O)₂—(C1-C3 alkylene)-;
Rₕ is H, C1-C5 alkyl, C1-C5 alkoxy, C1-C5 haloalkyl, halogen, C3-C6 cycloalkyl, phenoxy, phenyl, —(C1-C5 alkylene)-phenyl, -phenylen-O—(C1-C5 alkyl), -phenylen-C(═O), -phenylen-piperazinyl, 4- to 6-membered heterocycloalkyl including 1 to 3 heteroatoms of at least one selected from N, O and S, 5- to 10-membered heteroaryl including 1 to 3 heteroatoms of at least one selected from N, O, and S,

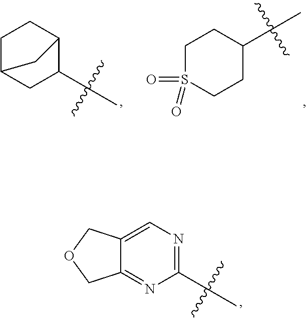 [SMILES: *C1CC2CCC1C2 ; *C1CCS(=O)(=O)CC1 ; *c1ncc2c(n1)COC2]

or —NR₆R₇;
R₆ and R₇ are each independently C1-C5 alkyl or C1-C5 haloalkyl; and
at least one H of Rₕ may be each independently substituted with C1-C5 alkyl, C1-C5 alkoxy, C1-C5 haloalkyl, OH or halogen.

In above formula 1, if Z₂ is C, then [symbol] and [symbol] may not be a double bond at the same time, and may not be a single bond at the same time.

In above formula 1

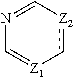

may be expressed as

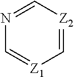

if [symbol] is a double bond, but may be explicitly viewed as substantially the same as the structure expressed as

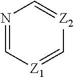

considering a definition of a resonance structure.

In above formula 1, if Z₂ is C, the compounds represented by formulas 1a and 1b below may mean substantially the same compound.

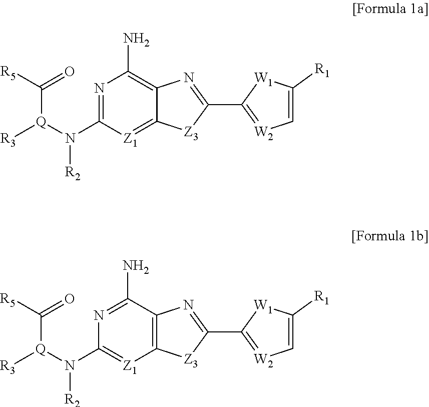

In above formula 1, a compound in which R₂ and R₃ are linked to form a ring may be represented by formula 1c below.

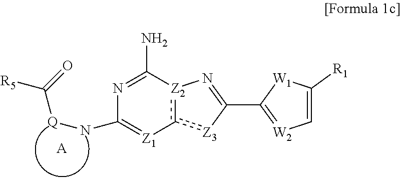

In above formula 1c, Q represents C—R₄ or N and a ring including Q and N represents a 4- to 6-membered N-containing heterocycloalkyl (in which at least one H of the N-containing heterocycloalkyl may be each independently substituted with C1-C5 alkyl or OH) or a 6- to 8-membered N-containing spiroheterocycloalkyl.

In the present invention, “alkyl” may mean a straight or branched saturated hydrocarbon group unless otherwise specified and, for example, “C1-C5 alkyl” may include methyl, ethyl, n-propyl, isopropyl, n-butyl, sec-butyl, tert-butyl, isobutyl, n-pentyl, sec-pentyl, tert-pentyl, isopentyl, sec-isopentyl, neo-pentyl, etc.

In the present invention, “alkylene” may mean a divalent functional group derived from the above-defined alkyl (including both straight and branched) unless otherwise specified and, for example, “C1-C3 alkylene” may include methylene (—CH₂—), ethylene (—CH₂CH₂—), n-propylene (—CH₂CH₂CH₂—), isopropylene (—CH(CH₃)—CH₂—) etc.

In the present invention, “hetero” may refer to a heteroatom or a heteroatomic group (that is, an atomic group containing a heteroatom) unless otherwise specified and may mean, for example, atoms such as oxygen (O), nitrogen (S), sulfur (S) and/or the like and an atomic group containing such a hetero atom.

In the present invention, “heteroaryl” may mean a heterocycle in which at least one carbon of an aromatic functional group is substituted with a heteroatom unless otherwise specified and the heteroatom may be O, N or S. For example, heteroaryl may include furyl, pyrrolyl, pyrazolyl, pyridyl, pyrimidyl, imidazolyl, triazolyl, triazinyl, pyridazinyl, pyrazinyl or the like, but is not limited thereto.

In the present invention, “heterocycloalkyl” may mean a cyclic alkyl group in which at least one carbon constituting a ring is substituted with a heteroatom unless otherwise specified. The heteroatom may be, for example, O, N or S. For example, heterocycloalkyl may include piperidinyl, morpholinyl, thiamorpholinyl, pyrrolidinyl, imidazolidinyl, oxiranyl, oxetanyl, tetrahydrofuranyl, tetrahydropyranyl, etc., but is not limited thereto.

In the present invention, “spiroheterocycloalkyl” may be a double ring including two rings sharing only one carbon, in which at least one of the two rings includes a heteroatom. The heteroatom may be, for example, 0, N or S. When one of the two rings is an x-angled shape and the other is an y-angled shape (in which x and y are each an integer of 3 or more), it may be referred to as a (x+y−1)-membered spiroheterocycloalkyl. For example, spiroheterocycloalkyl may be a 7-membered 5-azaspiro[2,4]heptanyl.

In the present invention, “haloalkyl” may mean a functional group in which at least one hydrogen is substituted with halogen in the alkyl group defined above. Examples of haloalkyl may include CF₃, CF₂H, CH₂F, CH₂CH₂F, CH₂CF₃, C(CH₃)₂CF₃, etc.

In the present invention, “halogen” may be F, Cl, Br or I unless otherwise specified.

(2) The compound represented by formula 1, stereoisomers thereof or pharmaceutically acceptable salts thereof according to above (1):

In formula 1,
W₁, W₂, Z₁, Z₂, Z₃, Q, R₁, R₂, R₃, R₄, [symbol] and [symbol] are each the same as defined above,
if W₁ is O, then W₂ is CH;
if W₁ is S, then W₂ is N;
R₅ is
—NH—(CH₂)_{y}—R_{b} (in which y is any one integer of 1 to 3, and R_{b} is a 5- or 6-membered heterocycloalkyl including O);

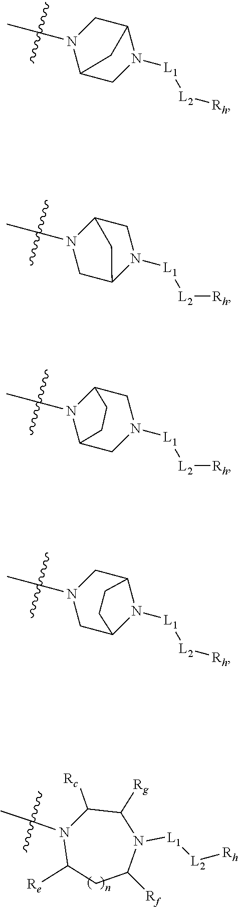

(in which n is 0 or 1, R_{c}, Rₑ, R_{f} and R_{g} are each independently H or C1-C5 alkyl) or

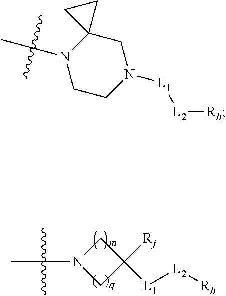

(in which m and q are each independently any one integer of 0 to 3, m and q may not be 0 at the same time, and Rⱼ is H or halogen);

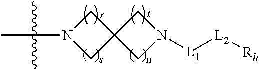

(in which r, s, t and u are each independently 1 or 2)

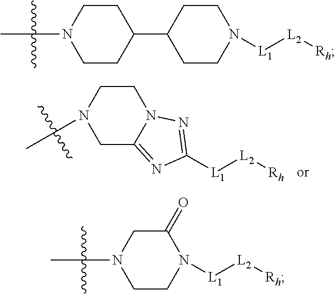

in above R₅, L₁, L₂ and Rₕ are each the same as defined above.

(3) The compound represented by formula 1, stereoisomers thereof or pharmaceutically acceptable salts thereof according to above (1) or (2):

In formula 1, W₁, W₂, Z₁, Z₂, Z₃, [symbol] and [symbol] are each the same as defined above;
Q is C—R₄;
R₁ and R₂ are each H;
R₃ is H or -La-Ra (in which La is a single bond or C1-C3 alkylene; Ra is C1-C5 alkyl, C3-C6 cycloalkyl,

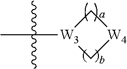

(a and b are each independently 1 or 2, W₃ is CH or N, W₄ is CH₂ or O, in which if W₃ is CH, then W₄ is not CH₂), phenyl or -phenylen-O-benzyl, and if Ra is C1-C5 alkyl or phenyl, then at least one of each H may be substituted with —OH or C1-C5 alkoxy;
R₄ is H or C1-C5 alkyl;
R₅ is

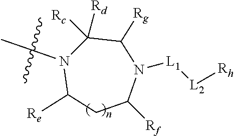

(in which n is 0 or 1, and R_{c}, R_{d}, Rₑ, R_{f} and R_{g} are each independently H or C1-C5 alkyl, but two selected from R_{c}, R_{d}, Rₑ, R_{f} and R_{g} may be linked to form CH₂ or CH₂—CH₂;

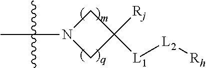

(in which m and q are each independently any one integer of 0 to 3, and Rⱼ is H or halogen);

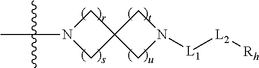

(in which r, s, t and u are each independently 1 or 2);

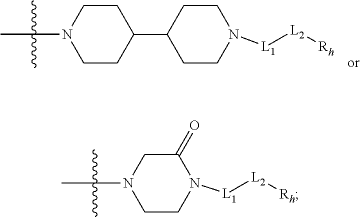

in above R₅,
L₁ is a single bond or C1-C3 alkylene;
L₂ is a single bond, —C(═O)— or —S(═O)₂—;
Rₕ is H, C1-C5 alkyl, C1-C5 alkoxy, C1-C5 haloalkyl, C3-C6 cycloalkyl, phenoxy, phenyl, 5- or 6-membered heterocycloalkyl including 1 to 3 heteroatoms of at least one selected from N and 0, or 5- or 6-membered heteroaryl including 1 to 3 heteroatoms of at least one selected from N and S; and
at least one H of Rₕ may be each independently substituted with C1-C5 alkoxy, C1-C5 haloalkyl, OH or halogen.

(4) The compound represented by formula 1, stereoisomers thereof or pharmaceutically acceptable salts thereof according to any one of above (1), (2), and (3):

In formula 1, W₁, W₂, Z₁, Z₂, Z₃, R₁, [symbol] and [symbol] are each the same as defined above,
Q is C—R₄ or N;
R₂ and R₃ are linked with each other to form 4- to 6-membered N-containing heterocycloalkyl (in which at least one H of the N-containing heterocycloalkyl may be each independently substituted with C1-C5 alkyl or OH), or a 6- to 8-membered N-containing spiroheterocycloalkyl;
R₄ is H or C1-C5 alkyl;
R₅ is
—NH—(CH₂)_{y}—R_{b} (in which y is any one integer of 1 to 3, and R_{b} is a 5- or 6-membered heterocycloalkyl including O);

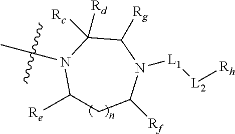

(in which n is 0 or 1, and R_{c}, R_{d}, Rₑ, R_{f} and R_{g} are each independently H or C1-C5 alkyl, but two selected from R_{c}, R_{d}, Rₑ, R_{f} and R_{g} may be linked to form CH₂ or CH₂—CH₂);

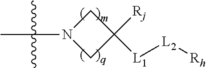

(in which m and q are each independently any one integer of 0 to 3, m and q may not be 0 at the same time, and Rⱼ is H or halogen);

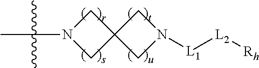

(in which r, s, t and u are each independently 1 or 2);

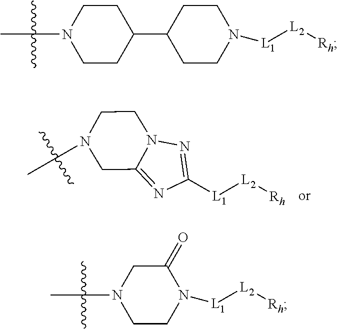

in above R₅,
L₁ is a single bond or C1-C3 alkylene;
L₂ is a single bond, —C(═O)—, —C(═O)NH—, —C(═O)—N(C1-C5 alkyl)-, —C(═O)—NH(C1-C5 alkylene)-, —S(═O)₂— or —S(═O)₂—(C1-C3 alkylene)-;
Rₕ is H, C1-C5 alkyl, C1-C5 alkoxy, C1-C5 haloalkyl, halogen, C3-C6 cycloalkyl, phenoxy, phenyl, —(C1-C3 alkylene)-phenyl, -phenylen-O—(C1-C5 alkyl), -phenylen-C(═O)—, -phenylen-piperazinyl, 4- to 6-membered heterocycloalkyl including 1 to 3 heteroatoms of at least one selected from N, O and S, 5- to 10-membered heteroaryl including 1 to 3 heteroatoms of at least one selected from N, O, and S,

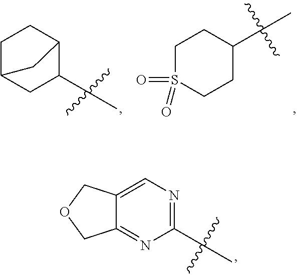 [SMILES: *C1CC2CCC1C2 ; *C1CCS(=O)(=O)CC1 ; *c1ncc2c(n1)COC2]

or —NR₆R₇;
R₆ and R₇ are each independently C1-C5 alkyl or C1-C5 haloalkyl; and
at least one H of Rₕ may be each independently substituted with C1-C5 alkyl, C1-C5 alkoxy, C1-C5 haloalkyl, OH or halogen.

(5) The compound represented by formula 1, stereoisomers thereof or pharmaceutically acceptable salts thereof according to any one of above (1), (2), (3), and (4), in which the compound may be at least one compound selected from the compounds shown in the table 1 below.

TABLE 1

| Example | |
|---|---|
| No. | Compound Strcutre |
| 1 | 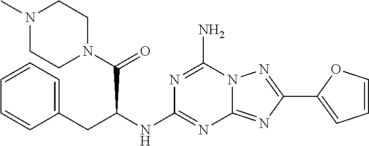 [SMILES: CN1CCN(C(=O)[C@H](Cc2ccccc2)Nc2nc(N)n3nc(-c4ccco4)nc3n2)CC1] |
| 2 | 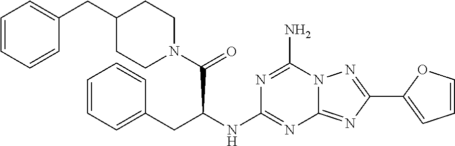 [SMILES: Nc1nc(N[C@@H](Cc2ccccc2)C(=O)N2CCC(Cc3ccccc3)CC2)nc2nc(-c3ccco3)nn12] |
| 3 | 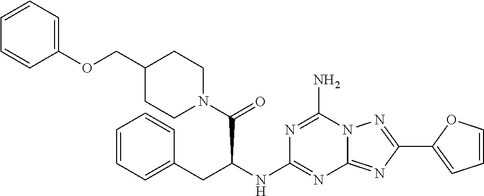 [SMILES: Nc1nc(N[C@@H](Cc2ccccc2)C(=O)N2CCC(COc3ccccc3)CC2)nc2nc(-c3ccco3)nn12] |
| 4 | 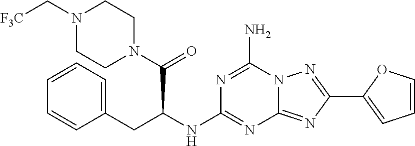 [SMILES: Nc1nc(N[C@@H](Cc2ccccc2)C(=O)N2CCN(CC(F)(F)F)CC2)nc2nc(-c3ccco3)nn12] |
| 5 | 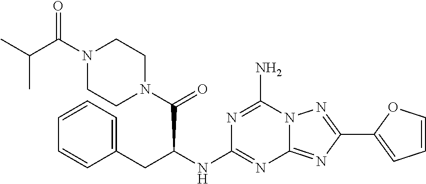 [SMILES: CC(C)C(=O)N1CCN(C(=O)[C@H](Cc2ccccc2)Nc2nc(N)n3nc(-c4ccco4)nc3n2)CC1] |
| 6 | 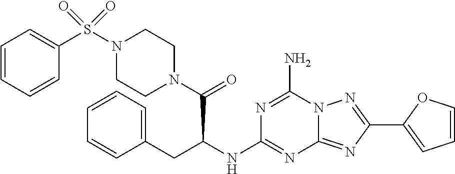 [SMILES: Nc1nc(N[C@@H](Cc2ccccc2)C(=O)N2CCN(S(=O)(=O)c3ccccc3)CC2)nc2nc(-c3ccco3)nn12] |
| 7 | 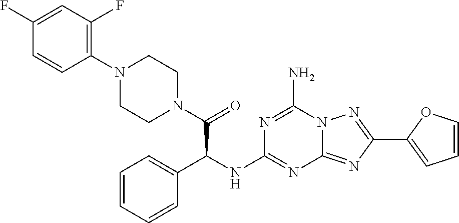 [SMILES: Nc1nc(N[C@H](C(=O)N2CCN(c3ccc(F)cc3F)CC2)c2ccccc2)nc2nc(-c3ccco3)nn12] |
| 8 | 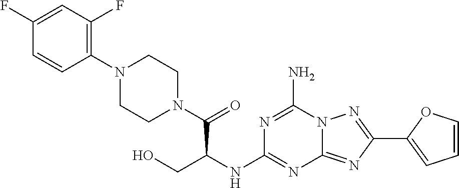 [SMILES: Nc1nc(N[C@@H](CO)C(=O)N2CCN(c3ccc(F)cc3F)CC2)nc2nc(-c3ccco3)nn12] |
| 9 | 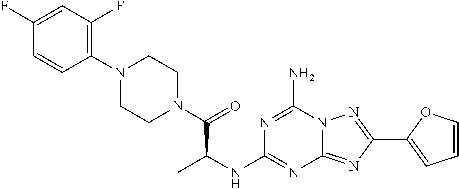 [SMILES: C[C@H](Nc1nc(N)n2nc(-c3ccco3)nc2n1)C(=O)N1CCN(c2ccc(F)cc2F)CC1] |
| 10 | 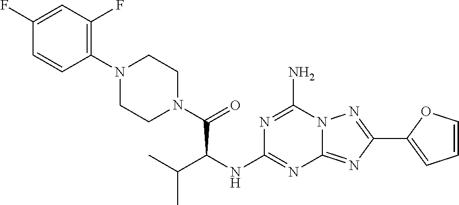 [SMILES: CC(C)[C@H](Nc1nc(N)n2nc(-c3ccco3)nc2n1)C(=O)N1CCN(c2ccc(F)cc2F)CC1] |
| 11 | 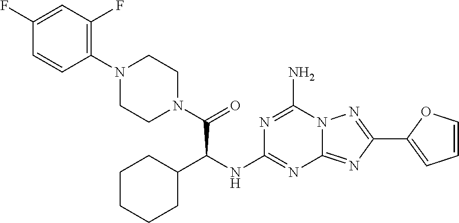 [SMILES: Nc1nc(N[C@H](C(=O)N2CCN(c3ccc(F)cc3F)CC2)C2CCCCC2)nc2nc(-c3ccco3)nn12] |
| 12 | 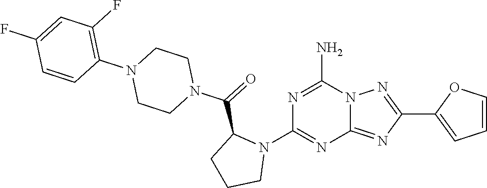 [SMILES: Nc1nc(N2CCC[C@H]2C(=O)N2CCN(c3ccc(F)cc3F)CC2)nc2nc(-c3ccco3)nn12] |
| 13 | 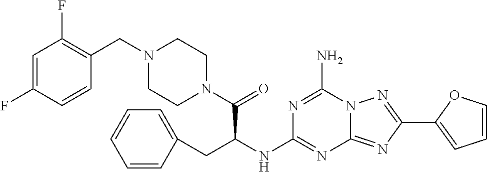 [SMILES: Nc1nc(N[C@@H](Cc2ccccc2)C(=O)N2CCN(Cc3ccc(F)cc3F)CC2)nc2nc(-c3ccco3)nn12] |
| 14 | 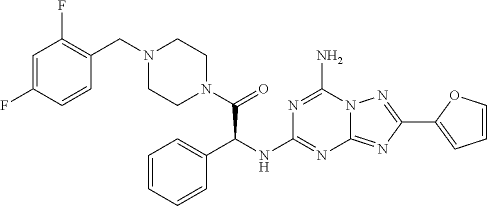 [SMILES: Nc1nc(N[C@H](C(=O)N2CCN(Cc3ccc(F)cc3F)CC2)c2ccccc2)nc2nc(-c3ccco3)nn12] |
| 15 | 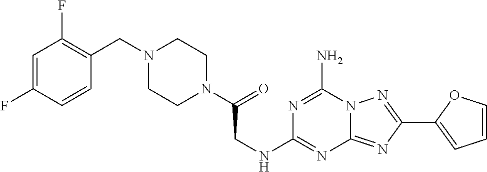 |
| 16 | 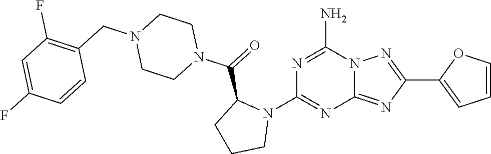 [SMILES: Nc1nc(N2CCC[C@H]2C(=O)N2CCN(Cc3ccc(F)cc3F)CC2)nc2nc(-c3ccco3)nn12] |
| 17 | 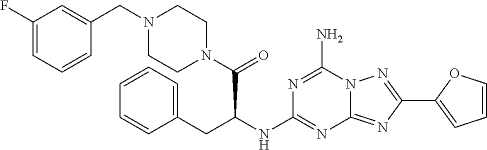 [SMILES: Nc1nc(N[C@@H](Cc2ccccc2)C(=O)N2CCN(Cc3cccc(F)c3)CC2)nc2nc(-c3ccco3)nn12] |
| 18 | 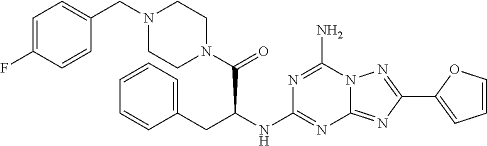 [SMILES: Nc1nc(N[C@@H](Cc2ccccc2)C(=O)N2CCN(Cc3ccc(F)cc3)CC2)nc2nc(-c3ccco3)nn12] |
| 19 | 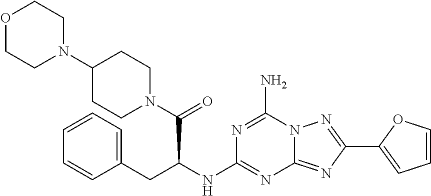 [SMILES: Nc1nc(N[C@@H](Cc2ccccc2)C(=O)N2CCC(N3CCOCC3)CC2)nc2nc(-c3ccco3)nn12] |
| 20 | 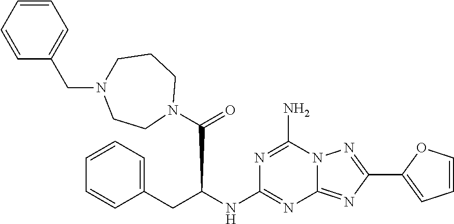 [SMILES: Nc1nc(N[C@@H](Cc2ccccc2)C(=O)N2CCCN(Cc3ccccc3)CC2)nc2nc(-c3ccco3)nn12] |
| 21 | 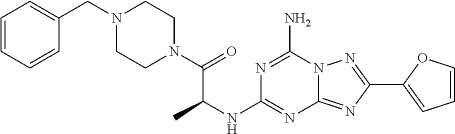 [SMILES: C[C@H](Nc1nc(N)n2nc(-c3ccco3)nc2n1)C(=O)N1CCN(Cc2ccccc2)CC1] |
| 22 | 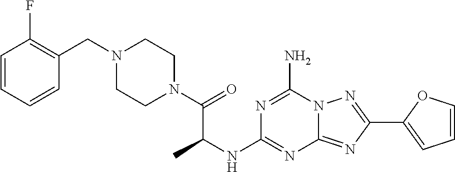 [SMILES: C[C@H](Nc1nc(N)n2nc(-c3ccco3)nc2n1)C(=O)N1CCN(Cc2ccccc2F)CC1] |
| 23 | 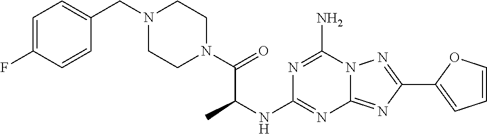 [SMILES: C[C@H](Nc1nc(N)n2nc(-c3ccco3)nc2n1)C(=O)N1CCN(Cc2ccc(F)cc2)CC1] |
| 24 | 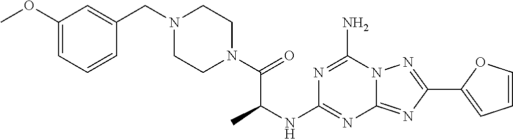 [SMILES: COc1cccc(CN2CCN(C(=O)[C@H](C)Nc3nc(N)n4nc(-c5ccco5)nc4n3)CC2)c1] |
| 25 | 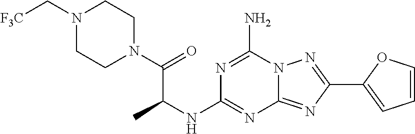 [SMILES: C[C@H](Nc1nc(N)n2nc(-c3ccco3)nc2n1)C(=O)N1CCN(CC(F)(F)F)CC1] |
| 26 | 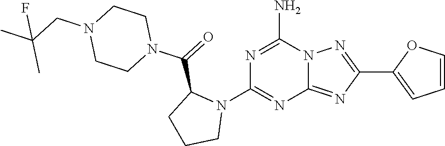 [SMILES: CC(C)(F)CN1CCN(C(=O)[C@@H]2CCCN2c2nc(N)n3nc(-c4ccco4)nc3n2)CC1] |
| 27 | 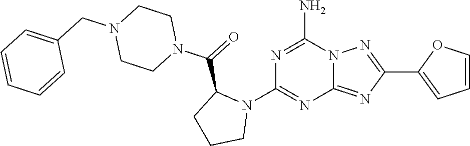 [SMILES: Nc1nc(N2CCC[C@H]2C(=O)N2CCN(Cc3ccccc3)CC2)nc2nc(-c3ccco3)nn12] |
| 28 | 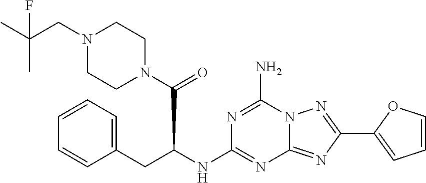 [SMILES: CC(C)(F)CN1CCN(C(=O)[C@H](Cc2ccccc2)Nc2nc(N)n3nc(-c4ccco4)nc3n2)CC1] |
| 29 | 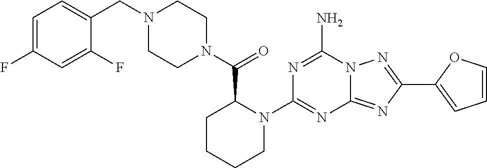 [SMILES: Nc1nc(N2CCCC[C@H]2C(=O)N2CCN(Cc3ccc(F)cc3F)CC2)nc2nc(-c3ccco3)nn12] |
| 30 | 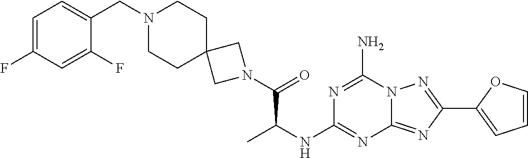 [SMILES: C[C@H](Nc1nc(N)n2nc(-c3ccco3)nc2n1)C(=O)N1CC2(CCN(Cc3ccc(F)cc3F)CC2)C1] |
| 31 | 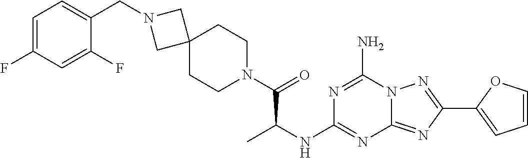 [SMILES: C[C@H](Nc1nc(N)n2nc(-c3ccco3)nc2n1)C(=O)N1CCC2(CC1)CN(Cc1ccc(F)cc1F)C2] |
| 32 | 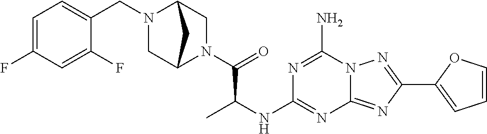 [SMILES: C[C@H](Nc1nc(N)n2nc(-c3ccco3)nc2n1)C(=O)N1C[C@@H]2C[C@H]1CN2Cc1ccc(F)cc1F] |
| 37 | 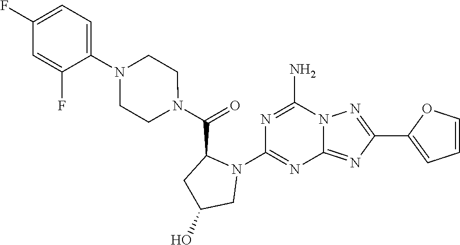 [SMILES: Nc1nc(N2C[C@H](O)C[C@H]2C(=O)N2CCN(c3ccc(F)cc3F)CC2)nc2nc(-c3ccco3)nn12] |
| 40 | 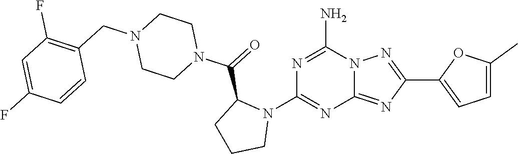 [SMILES: Cc1ccc(-c2nc3nc(N4CCC[C@H]4C(=O)N4CCN(Cc5ccc(F)cc5F)CC4)nc(N)n3n2)o1] |
| 41 | 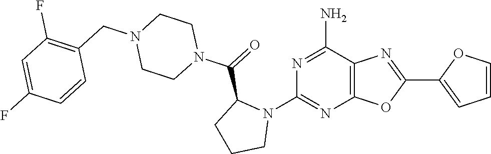 [SMILES: Nc1nc(N2CCC[C@H]2C(=O)N2CCN(Cc3ccc(F)cc3F)CC2)nc2oc(-c3ccco3)nc12] |
| 42 | 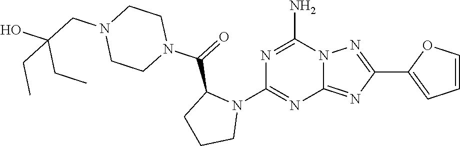 [SMILES: CCC(O)(CC)CN1CCN(C(=O)[C@@H]2CCCN2c2nc(N)n3nc(-c4ccco4)nc3n2)CC1] |
| 43 | 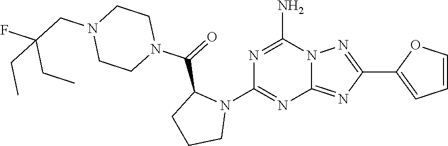 [SMILES: CCC(F)(CC)CN1CCN(C(=O)[C@@H]2CCCN2c2nc(N)n3nc(-c4ccco4)nc3n2)CC1] |
| 44 | 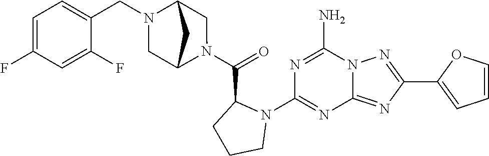 [SMILES: Nc1nc(N2CCC[C@H]2C(=O)N2C[C@@H]3C[C@H]2CN3Cc2ccc(F)cc2F)nc2nc(-c3ccco3)nn12] |
| 45 | 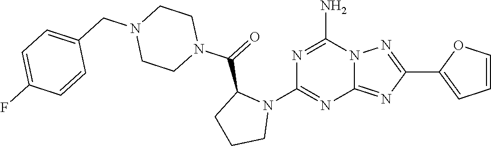 [SMILES: Nc1nc(N2CCC[C@H]2C(=O)N2CCN(Cc3ccc(F)cc3)CC2)nc2nc(-c3ccco3)nn12] |
| 46 | 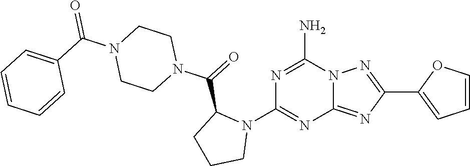 [SMILES: Nc1nc(N2CCC[C@H]2C(=O)N2CCN(C(=O)c3ccccc3)CC2)nc2nc(-c3ccco3)nn12] |
| 47 | 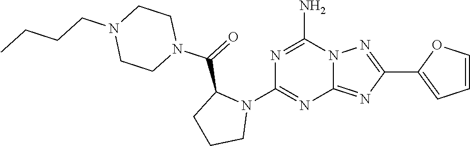 [SMILES: CCCCN1CCN(C(=O)[C@@H]2CCCN2c2nc(N)n3nc(-c4ccco4)nc3n2)CC1] |
| 48 | 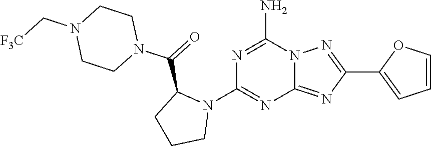 [SMILES: Nc1nc(N2CCC[C@H]2C(=O)N2CCN(CC(F)(F)F)CC2)nc2nc(-c3ccco3)nn12] |
| 49 | 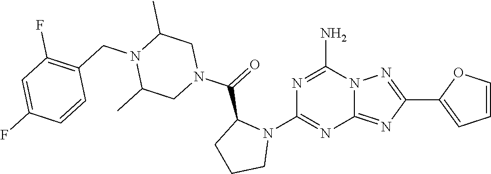 [SMILES: CC1CN(C(=O)[C@@H]2CCCN2c2nc(N)n3nc(-c4ccco4)nc3n2)CC(C)N1Cc1ccc(F)cc1F] |
| 50 | 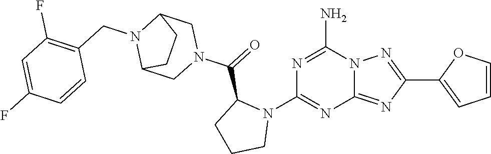 [SMILES: Nc1nc(N2CCC[C@H]2C(=O)N2CC3CCC(C2)N3Cc2ccc(F)cc2F)nc2nc(-c3ccco3)nn12] |
| 51 | 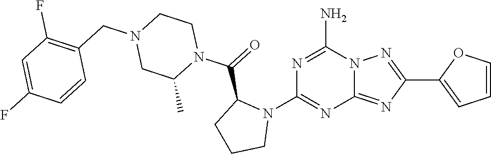 [SMILES: C[C@@H]1CN(Cc2ccc(F)cc2F)CCN1C(=O)[C@@H]1CCCN1c1nc(N)n2nc(-c3ccco3)nc2n1] |
| 52 | 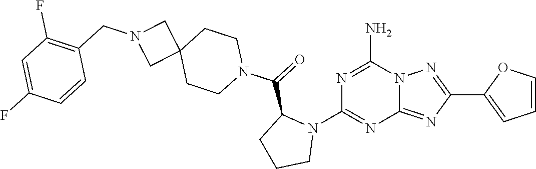 [SMILES: Nc1nc(N2CCC[C@H]2C(=O)N2CCC3(CC2)CN(Cc2ccc(F)cc2F)C3)nc2nc(-c3ccco3)nn12] |
| 53 | 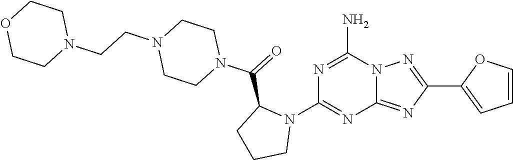 [SMILES: Nc1nc(N2CCC[C@H]2C(=O)N2CCN(CCN3CCOCC3)CC2)nc2nc(-c3ccco3)nn12] |
| 54 | 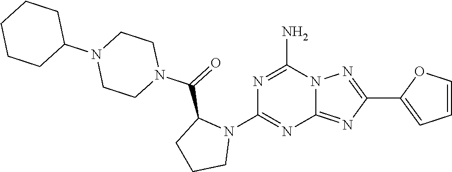 [SMILES: Nc1nc(N2CCC[C@H]2C(=O)N2CCN(C3CCCCC3)CC2)nc2nc(-c3ccco3)nn12] |
| 55 | 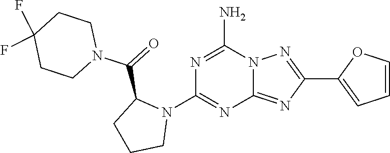 [SMILES: Nc1nc(N2CCC[C@H]2C(=O)N2CCC(F)(F)CC2)nc2nc(-c3ccco3)nn12] |
| 56 | 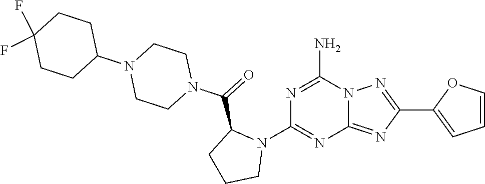 [SMILES: Nc1nc(N2CCC[C@H]2C(=O)N2CCN(C3CCC(F)(F)CC3)CC2)nc2nc(-c3ccco3)nn12] |
| 57 | 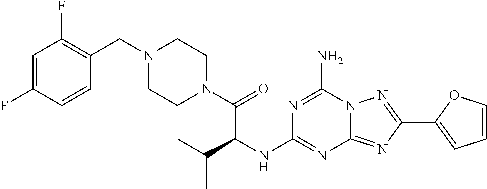 [SMILES: CC(C)[C@H](Nc1nc(N)n2nc(-c3ccco3)nc2n1)C(=O)N1CCN(Cc2ccc(F)cc2F)CC1] |
| 58 | 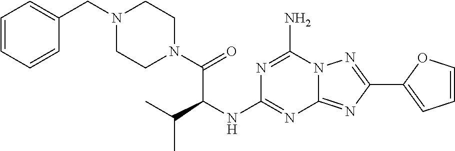 [SMILES: CC(C)[C@H](Nc1nc(N)n2nc(-c3ccco3)nc2n1)C(=O)N1CCN(Cc2ccccc2)CC1] |
| 59 | 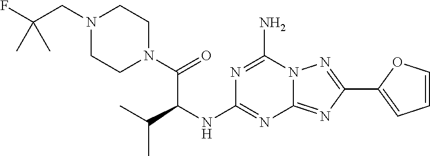 [SMILES: CC(C)[C@H](Nc1nc(N)n2nc(-c3ccco3)nc2n1)C(=O)N1CCN(CC(C)(C)F)CC1] |
| 60 | 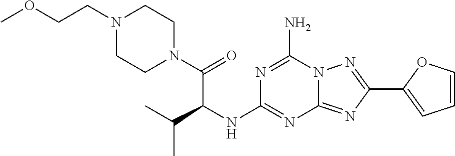 [SMILES: COCCN1CCN(C(=O)[C@@H](Nc2nc(N)n3nc(-c4ccco4)nc3n2)C(C)C)CC1] |
| 61 | 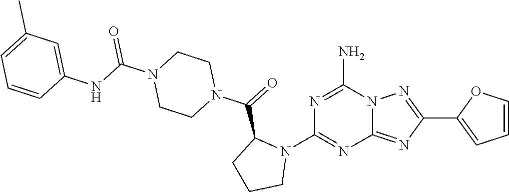 [SMILES: Cc1cccc(NC(=O)N2CCN(C(=O)[C@@H]3CCCN3c3nc(N)n4nc(-c5ccco5)nc4n3)CC2)c1] |
| 62 | 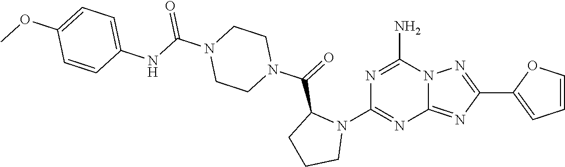 [SMILES: COc1ccc(NC(=O)N2CCN(C(=O)[C@@H]3CCCN3c3nc(N)n4nc(-c5ccco5)nc4n3)CC2)cc1] |
| 63 | 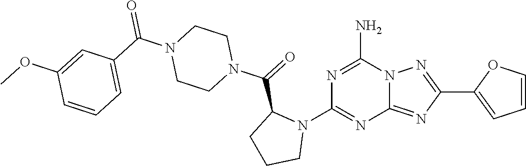 [SMILES: COc1cccc(C(=O)N2CCN(C(=O)[C@@H]3CCCN3c3nc(N)n4nc(-c5ccco5)nc4n3)CC2)c1] |
| 64 | 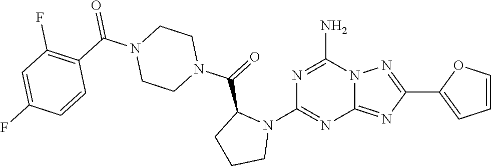 [SMILES: Nc1nc(N2CCC[C@H]2C(=O)N2CCN(C(=O)c3ccc(F)cc3F)CC2)nc2nc(-c3ccco3)nn12] |
| 65 | 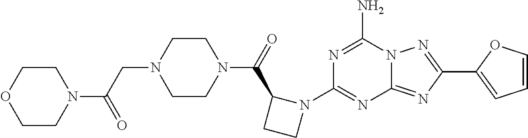 [SMILES: Nc1nc(N2CC[C@H]2C(=O)N2CCN(CC(=O)N3CCOCC3)CC2)nc2nc(-c3ccco3)nn12] |
| 66 | 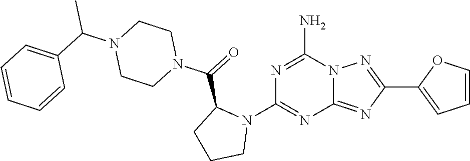 [SMILES: CC(c1ccccc1)N1CCN(C(=O)[C@@H]2CCCN2c2nc(N)n3nc(-c4ccco4)nc3n2)CC1] |
| 67 | 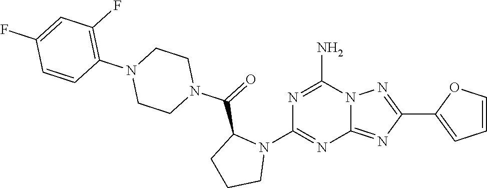 [SMILES: Nc1nc(N2CCC[C@H]2C(=O)N2CCN(c3ccc(F)cc3F)CC2)nc2nc(-c3ccco3)nn12] |
| 68 | 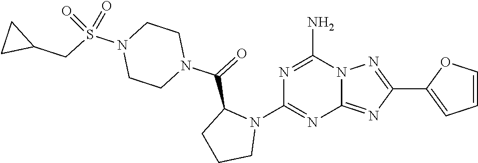 [SMILES: Nc1nc(N2CCC[C@H]2C(=O)N2CCN(S(=O)(=O)CC3CC3)CC2)nc2nc(-c3ccco3)nn12] |
| 69 | 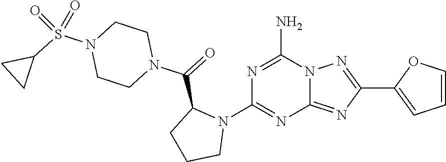 [SMILES: Nc1nc(N2CCC[C@H]2C(=O)N2CCN(S(=O)(=O)C3CC3)CC2)nc2nc(-c3ccco3)nn12] |
| 70 | 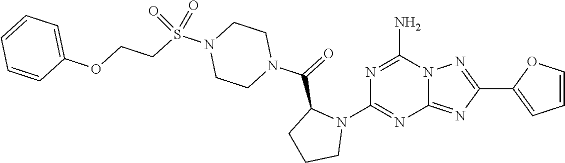 [SMILES: Nc1nc(N2CCC[C@H]2C(=O)N2CCN(S(=O)(=O)CCOc3ccccc3)CC2)nc2nc(-c3ccco3)nn12] |
| 71 | 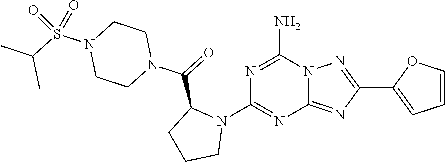 [SMILES: CC(C)S(=O)(=O)N1CCN(C(=O)[C@@H]2CCCN2c2nc(N)n3nc(-c4ccco4)nc3n2)CC1] |
| 72 | 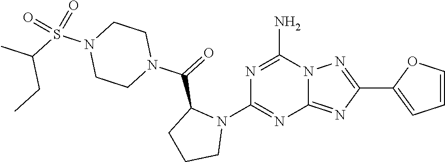 [SMILES: CCC(C)S(=O)(=O)N1CCN(C(=O)[C@@H]2CCCN2c2nc(N)n3nc(-c4ccco4)nc3n2)CC1] |
| 73 | 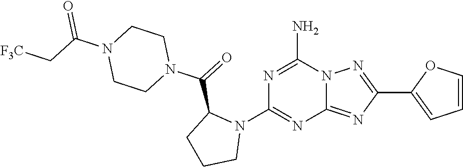 [SMILES: Nc1nc(N2CCC[C@H]2C(=O)N2CCN(C(=O)CC(F)(F)F)CC2)nc2nc(-c3ccco3)nn12] |
| 74 | 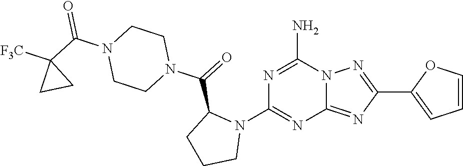 [SMILES: Nc1nc(N2CCC[C@H]2C(=O)N2CCN(C(=O)C3(C(F)(F)F)CC3)CC2)nc2nc(-c3ccco3)nn12] |
| 75 | 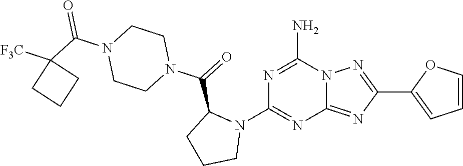 [SMILES: Nc1nc(N2CCC[C@H]2C(=O)N2CCN(C(=O)C3(C(F)(F)F)CCC3)CC2)nc2nc(-c3ccco3)nn12] |
| 76 | 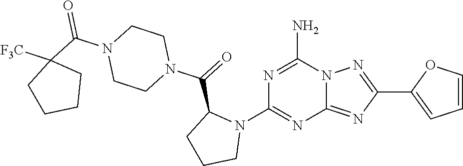 [SMILES: Nc1nc(N2CCC[C@H]2C(=O)N2CCN(C(=O)C3(C(F)(F)F)CCCC3)CC2)nc2nc(-c3ccco3)nn12] |
| 77 | 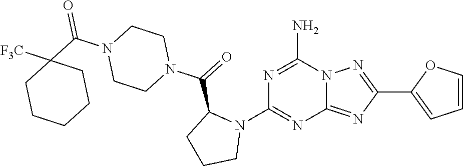 [SMILES: Nc1nc(N2CCC[C@H]2C(=O)N2CCN(C(=O)C3(C(F)(F)F)CCCCC3)CC2)nc2nc(-c3ccco3)nn12] |
| 78 | 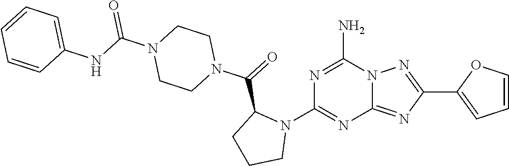 [SMILES: Nc1nc(N2CCC[C@H]2C(=O)N2CCN(C(=O)Nc3ccccc3)CC2)nc2nc(-c3ccco3)nn12] |
| 79 | 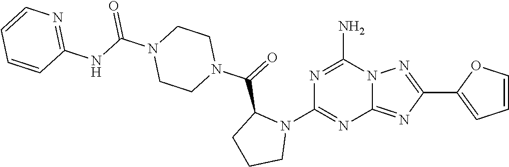 [SMILES: Nc1nc(N2CCC[C@H]2C(=O)N2CCN(C(=O)Nc3ccccn3)CC2)nc2nc(-c3ccco3)nn12] |
| 80 | 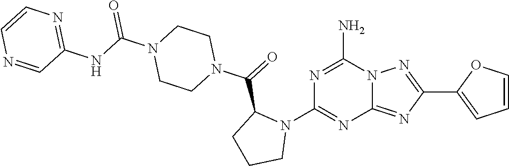 [SMILES: Nc1nc(N2CCC[C@H]2C(=O)N2CCN(C(=O)Nc3cnccn3)CC2)nc2nc(-c3ccco3)nn12] |
| 81 | 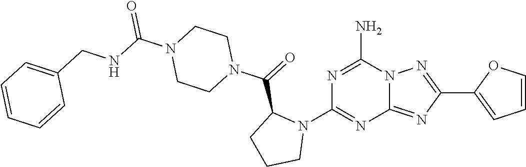 [SMILES: Nc1nc(N2CCC[C@H]2C(=O)N2CCN(C(=O)NCc3ccccc3)CC2)nc2nc(-c3ccco3)nn12] |
| 82 | 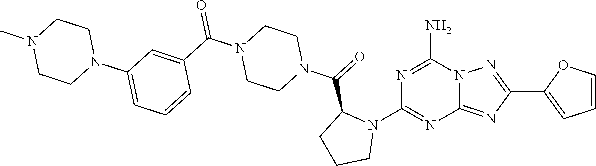 [SMILES: CN1CCN(c2cccc(C(=O)N3CCN(C(=O)[C@@H]4CCCN4c4nc(N)n5nc(-c6ccco6)nc5n4)CC3)c2)CC1] |
| 83 | 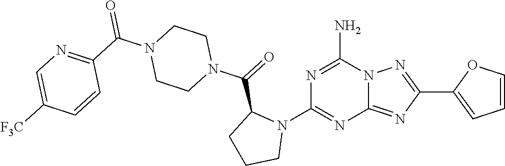 [SMILES: Nc1nc(N2CCC[C@H]2C(=O)N2CCN(C(=O)c3ccc(C(F)(F)F)cn3)CC2)nc2nc(-c3ccco3)nn12] |
| 84 | 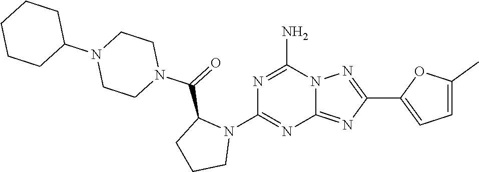 [SMILES: Cc1ccc(-c2nc3nc(N4CCC[C@H]4C(=O)N4CCN(C5CCCCC5)CC4)nc(N)n3n2)o1] |
| 85 | 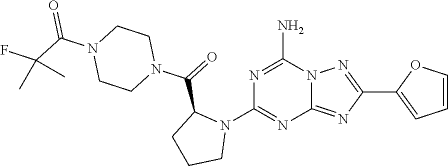 [SMILES: CC(C)(F)C(=O)N1CCN(C(=O)[C@@H]2CCCN2c2nc(N)n3nc(-c4ccco4)nc3n2)CC1] |
| 86 | 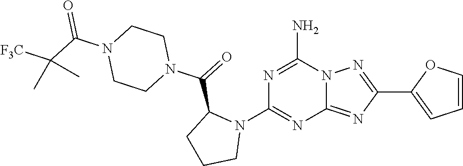 [SMILES: CC(C)(C(=O)N1CCN(C(=O)[C@@H]2CCCN2c2nc(N)n3nc(-c4ccco4)nc3n2)CC1)C(F)(F)F] |
| 87 | 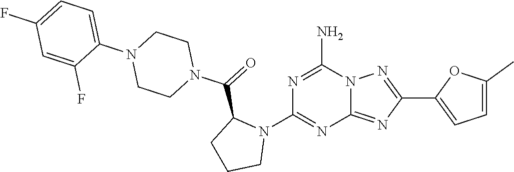 [SMILES: Cc1ccc(-c2nc3nc(N4CCC[C@H]4C(=O)N4CCN(c5ccc(F)cc5F)CC4)nc(N)n3n2)o1] |
| 88 | 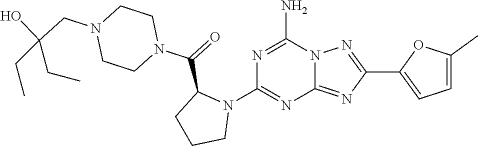 [SMILES: CCC(O)(CC)CN1CCN(C(=O)[C@@H]2CCCN2c2nc(N)n3nc(-c4ccc(C)o4)nc3n2)CC1] |
| 89 | 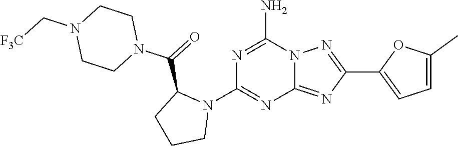 [SMILES: Cc1ccc(-c2nc3nc(N4CCC[C@H]4C(=O)N4CCN(CC(F)(F)F)CC4)nc(N)n3n2)o1] |
| 90 | 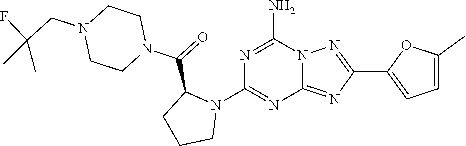 [SMILES: Cc1ccc(-c2nc3nc(N4CCC[C@H]4C(=O)N4CCN(CC(C)(C)F)CC4)nc(N)n3n2)o1] |
| 91 | 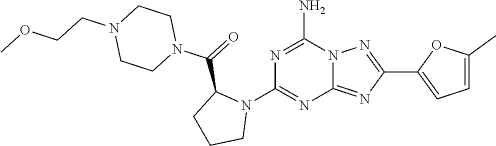 |
| 92 | 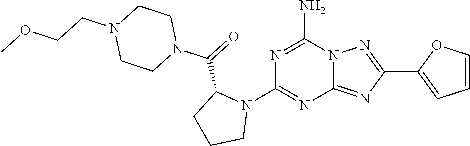 [SMILES: COCCN1CCN(C(=O)[C@H]2CCCN2c2nc(N)n3nc(-c4ccco4)nc3n2)CC1] |
| 93 | 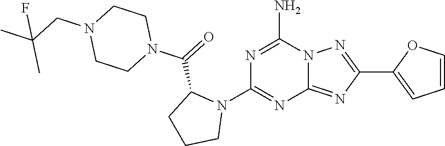 [SMILES: CC(C)(F)CN1CCN(C(=O)[C@H]2CCCN2c2nc(N)n3nc(-c4ccco4)nc3n2)CC1] |
| 94 | 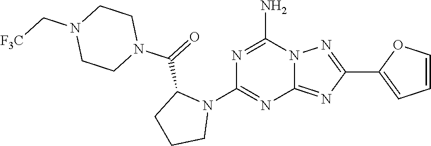 [SMILES: Nc1nc(N2CCC[C@@H]2C(=O)N2CCN(CC(F)(F)F)CC2)nc2nc(-c3ccco3)nn12] |
| 95 | 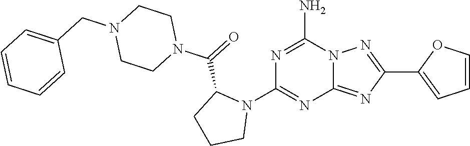 [SMILES: Nc1nc(N2CCC[C@@H]2C(=O)N2CCN(Cc3ccccc3)CC2)nc2nc(-c3ccco3)nn12] |
| 96 | 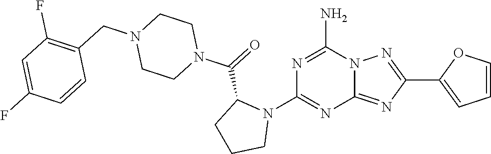 [SMILES: Nc1nc(N2CCC[C@@H]2C(=O)N2CCN(Cc3ccc(F)cc3F)CC2)nc2nc(-c3ccco3)nn12] |
| 97 | 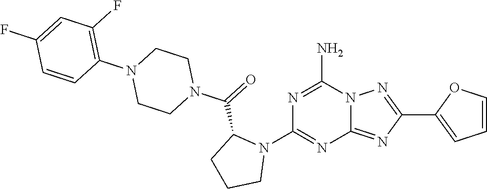 [SMILES: Nc1nc(N2CCC[C@@H]2C(=O)N2CCN(c3ccc(F)cc3F)CC2)nc2nc(-c3ccco3)nn12] |
| 98 | 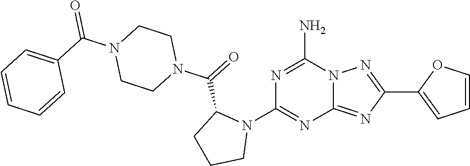 [SMILES: Nc1nc(N2CCC[C@@H]2C(=O)N2CCN(C(=O)c3ccccc3)CC2)nc2nc(-c3ccco3)nn12] |
| 99 | 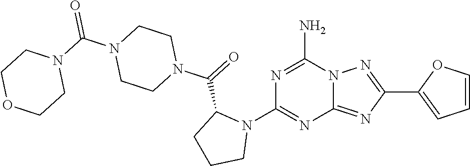 [SMILES: Nc1nc(N2CCC[C@@H]2C(=O)N2CCN(C(=O)N3CCOCC3)CC2)nc2nc(-c3ccco3)nn12] |
| 100 | 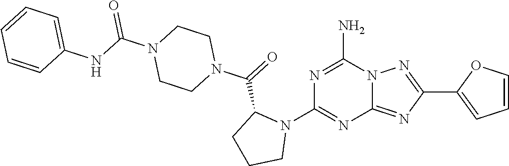 [SMILES: Nc1nc(N2CCC[C@@H]2C(=O)N2CCN(C(=O)Nc3ccccc3)CC2)nc2nc(-c3ccco3)nn12] |
| 101 | 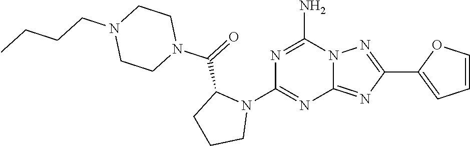 [SMILES: CCCCN1CCN(C(=O)[C@H]2CCCN2c2nc(N)n3nc(-c4ccco4)nc3n2)CC1] |
| 102 | 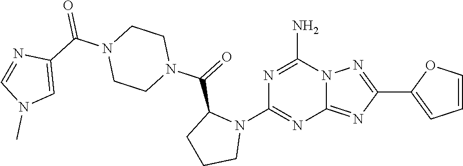 [SMILES: Cn1cnc(C(=O)N2CCN(C(=O)[C@@H]3CCCN3c3nc(N)n4nc(-c5ccco5)nc4n3)CC2)c1] |
| 103 | 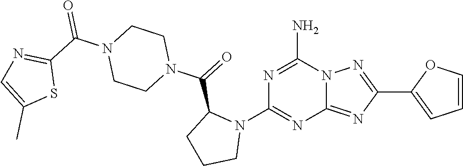 [SMILES: Cc1cnc(C(=O)N2CCN(C(=O)[C@@H]3CCCN3c3nc(N)n4nc(-c5ccco5)nc4n3)CC2)s1] |
| 104 | 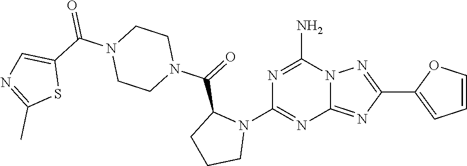 [SMILES: Cc1ncc(C(=O)N2CCN(C(=O)[C@@H]3CCCN3c3nc(N)n4nc(-c5ccco5)nc4n3)CC2)s1] |
| 105 | 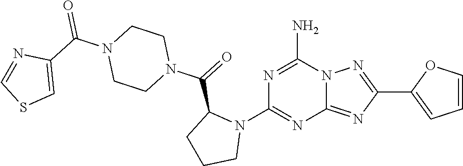 [SMILES: Nc1nc(N2CCC[C@H]2C(=O)N2CCN(C(=O)c3cscn3)CC2)nc2nc(-c3ccco3)nn12] |
| 106 | 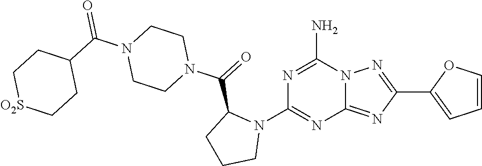 [SMILES: Nc1nc(N2CCC[C@H]2C(=O)N2CCN(C(=O)C3CCS(=O)(=O)CC3)CC2)nc2nc(-c3ccco3)nn12] |
| 107 | 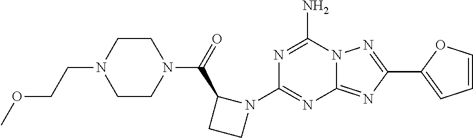 [SMILES: COCCN1CCN(C(=O)[C@@H]2CCN2c2nc(N)n3nc(-c4ccco4)nc3n2)CC1] |
| 108 | 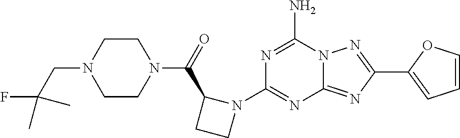 [SMILES: CC(C)(F)CN1CCN(C(=O)[C@@H]2CCN2c2nc(N)n3nc(-c4ccco4)nc3n2)CC1] |
| 109 | 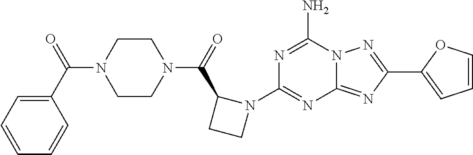 [SMILES: Nc1nc(N2CC[C@H]2C(=O)N2CCN(C(=O)c3ccccc3)CC2)nc2nc(-c3ccco3)nn12] |
| 110 | 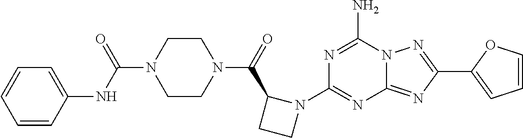 [SMILES: Nc1nc(N2CC[C@H]2C(=O)N2CCN(C(=O)Nc3ccccc3)CC2)nc2nc(-c3ccco3)nn12] |
| 111 | 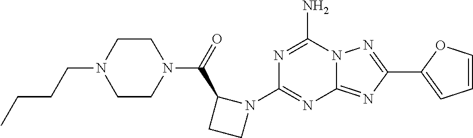 [SMILES: CCCCN1CCN(C(=O)[C@@H]2CCN2c2nc(N)n3nc(-c4ccco4)nc3n2)CC1] |
| 112 | 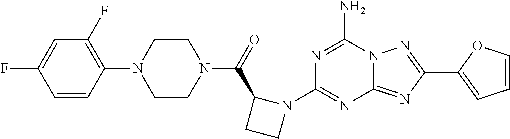 [SMILES: Nc1nc(N2CC[C@H]2C(=O)N2CCN(c3ccc(F)cc3F)CC2)nc2nc(-c3ccco3)nn12] |
| 113 | 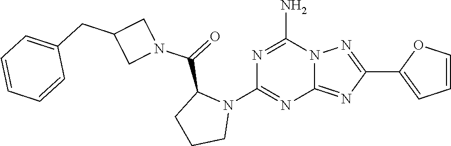 [SMILES: Nc1nc(N2CCC[C@H]2C(=O)N2CC(Cc3ccccc3)C2)nc2nc(-c3ccco3)nn12] |
| 114 | 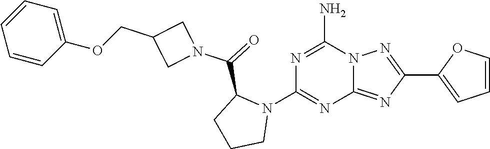 [SMILES: Nc1nc(N2CCC[C@H]2C(=O)N2CC(COc3ccccc3)C2)nc2nc(-c3ccco3)nn12] |
| 115 | 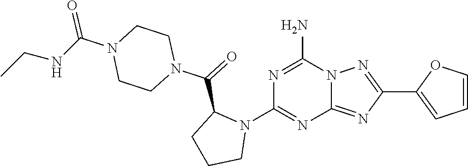 [SMILES: CCNC(=O)N1CCN(C(=O)[C@@H]2CCCN2c2nc(N)n3nc(-c4ccco4)nc3n2)CC1] |
| 116 | 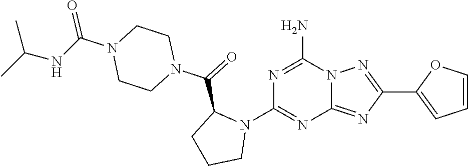 [SMILES: CC(C)NC(=O)N1CCN(C(=O)[C@@H]2CCCN2c2nc(N)n3nc(-c4ccco4)nc3n2)CC1] |
| 117 | 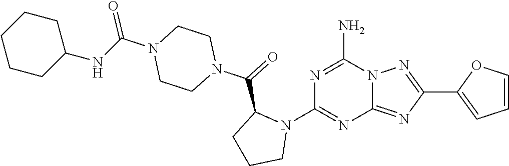 [SMILES: Nc1nc(N2CCC[C@H]2C(=O)N2CCN(C(=O)NC3CCCCC3)CC2)nc2nc(-c3ccco3)nn12] |
| 118 | 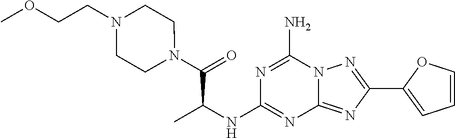 [SMILES: COCCN1CCN(C(=O)[C@H](C)Nc2nc(N)n3nc(-c4ccco4)nc3n2)CC1] |
| 119 | 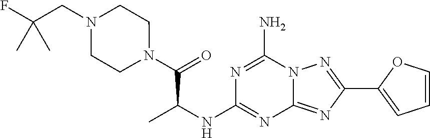 [SMILES: C[C@H](Nc1nc(N)n2nc(-c3ccco3)nc2n1)C(=O)N1CCN(CC(C)(C)F)CC1] |
| 120 | 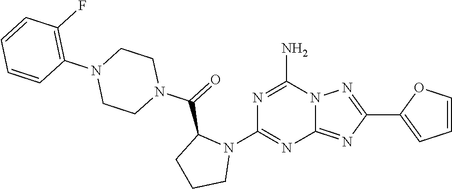 [SMILES: Nc1nc(N2CCC[C@H]2C(=O)N2CCN(c3ccccc3F)CC2)nc2nc(-c3ccco3)nn12] |
| 121 | 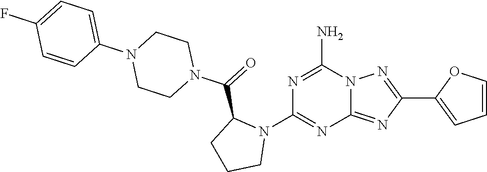 [SMILES: Nc1nc(N2CCC[C@H]2C(=O)N2CCN(c3ccc(F)cc3)CC2)nc2nc(-c3ccco3)nn12] |
| 122 | 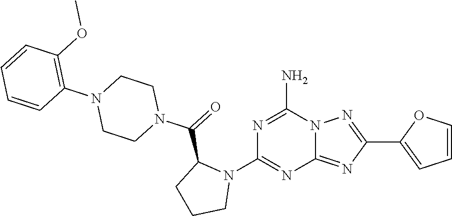 [SMILES: COc1ccccc1N1CCN(C(=O)[C@@H]2CCCN2c2nc(N)n3nc(-c4ccco4)nc3n2)CC1] |
| 123 | 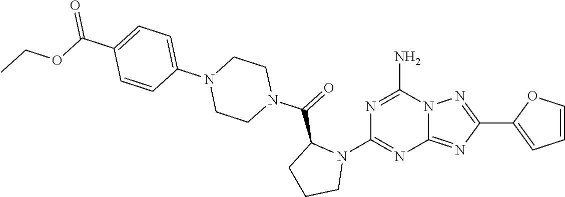 [SMILES: CCOC(=O)c1ccc(N2CCN(C(=O)[C@@H]3CCCN3c3nc(N)n4nc(-c5ccco5)nc4n3)CC2)cc1] |
| 124 | 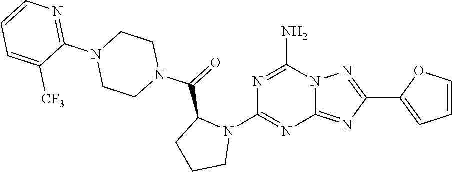 [SMILES: Nc1nc(N2CCC[C@H]2C(=O)N2CCN(c3ncccc3C(F)(F)F)CC2)nc2nc(-c3ccco3)nn12] |
| 125 | 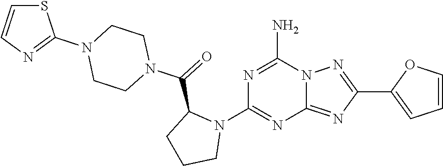 [SMILES: Nc1nc(N2CCC[C@H]2C(=O)N2CCN(c3nccs3)CC2)nc2nc(-c3ccco3)nn12] |
| 126 | 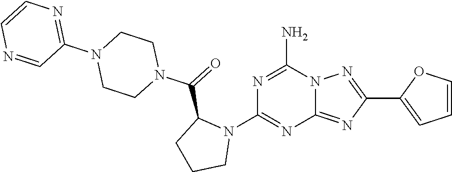 [SMILES: Nc1nc(N2CCC[C@H]2C(=O)N2CCN(c3cnccn3)CC2)nc2nc(-c3ccco3)nn12] |
| 127 | 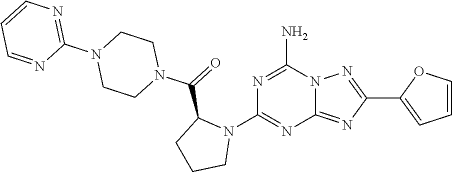 [SMILES: Nc1nc(N2CCC[C@H]2C(=O)N2CCN(c3ncccn3)CC2)nc2nc(-c3ccco3)nn12] |
| 128 | 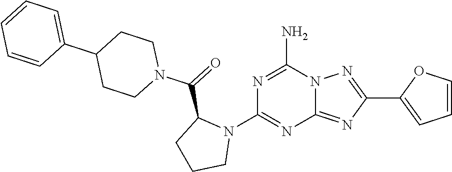 [SMILES: Nc1nc(N2CCC[C@H]2C(=O)N2CCC(c3ccccc3)CC2)nc2nc(-c3ccco3)nn12] |
| 129 | 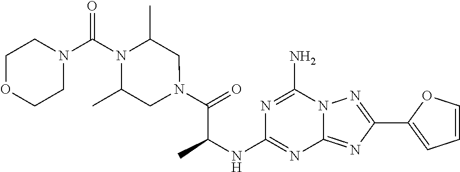 [SMILES: CC1CN(C(=O)[C@H](C)Nc2nc(N)n3nc(-c4ccco4)nc3n2)CC(C)N1C(=O)N1CCOCC1] |
| 130 | 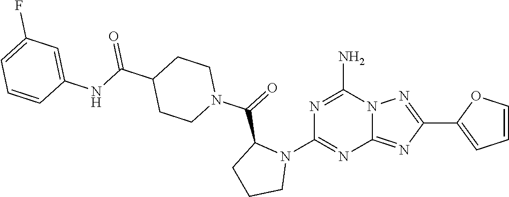 [SMILES: Nc1nc(N2CCC[C@H]2C(=O)N2CCC(C(=O)Nc3cccc(F)c3)CC2)nc2nc(-c3ccco3)nn12] |
| 131 | 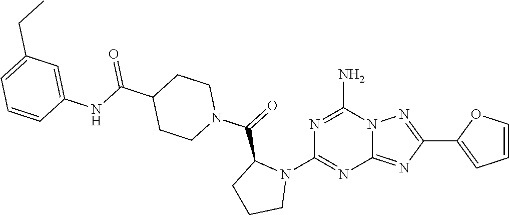 [SMILES: CCc1cccc(NC(=O)C2CCN(C(=O)[C@@H]3CCCN3c3nc(N)n4nc(-c5ccco5)nc4n3)CC2)c1] |
| 132 | 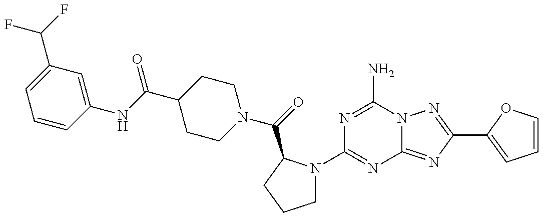 [SMILES: Nc1nc(N2CCC[C@H]2C(=O)N2CCC(C(=O)Nc3cccc(C(F)F)c3)CC2)nc2nc(-c3ccco3)nn12] |
| 133 | 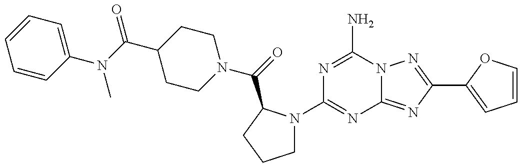 [SMILES: CN(C(=O)C1CCN(C(=O)[C@@H]2CCCN2c2nc(N)n3nc(-c4ccco4)nc3n2)CC1)c1ccccc1] |
| 134 | 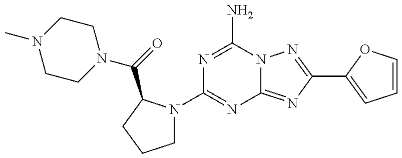 [SMILES: CN1CCN(C(=O)[C@@H]2CCCN2c2nc(N)n3nc(-c4ccco4)nc3n2)CC1] |
| 135 | 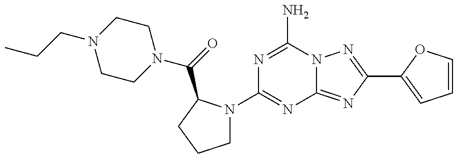 [SMILES: CCCN1CCN(C(=O)[C@@H]2CCCN2c2nc(N)n3nc(-c4ccco4)nc3n2)CC1] |
| 136 | 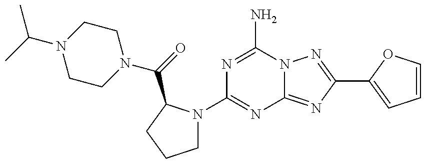 [SMILES: CC(C)N1CCN(C(=O)[C@@H]2CCCN2c2nc(N)n3nc(-c4ccco4)nc3n2)CC1] |
| 137 | 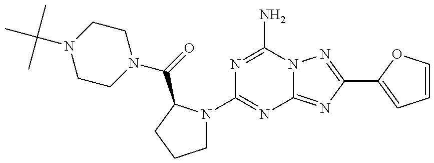 [SMILES: CC(C)(C)N1CCN(C(=O)[C@@H]2CCCN2c2nc(N)n3nc(-c4ccco4)nc3n2)CC1] |
| 138 | 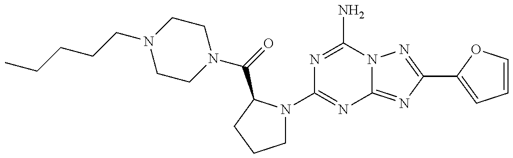 [SMILES: CCCCCN1CCN(C(=O)[C@@H]2CCCN2c2nc(N)n3nc(-c4ccco4)nc3n2)CC1] |
| 139 | 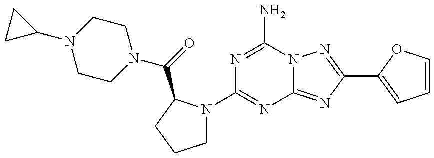 [SMILES: Nc1nc(N2CCC[C@H]2C(=O)N2CCN(C3CC3)CC2)nc2nc(-c3ccco3)nn12] |
| 140 | 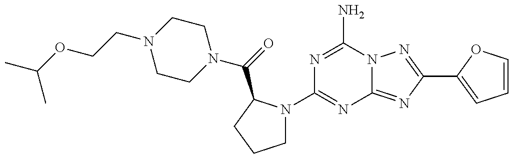 [SMILES: CC(C)OCCN1CCN(C(=O)[C@@H]2CCCN2c2nc(N)n3nc(-c4ccco4)nc3n2)CC1] |
| 141 | 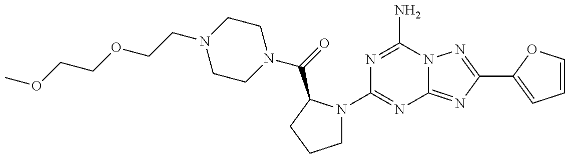 [SMILES: COCCOCCN1CCN(C(=O)[C@@H]2CCCN2c2nc(N)n3nc(-c4ccco4)nc3n2)CC1] |
| 142 | 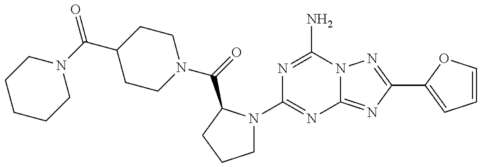 [SMILES: Nc1nc(N2CCC[C@H]2C(=O)N2CCC(C(=O)N3CCCCC3)CC2)nc2nc(-c3ccco3)nn12] |
| 143 | 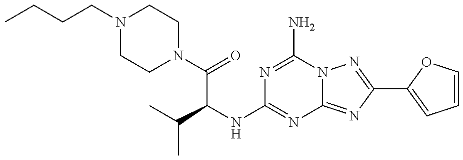 [SMILES: CCCCN1CCN(C(=O)[C@@H](Nc2nc(N)n3nc(-c4ccco4)nc3n2)C(C)C)CC1] |
| 144 | 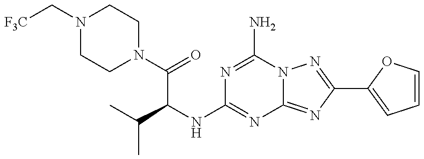 [SMILES: CC(C)[C@H](Nc1nc(N)n2nc(-c3ccco3)nc2n1)C(=O)N1CCN(CC(F)(F)F)CC1] |
| 145 | 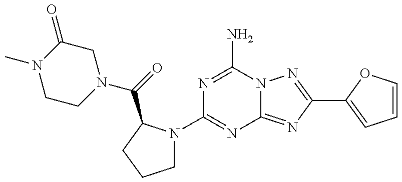 [SMILES: CN1CCN(C(=O)[C@@H]2CCCN2c2nc(N)n3nc(-c4ccco4)nc3n2)CC1=O] |
| 146 | 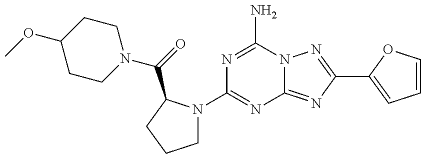 [SMILES: COC1CCN(C(=O)[C@@H]2CCCN2c2nc(N)n3nc(-c4ccco4)nc3n2)CC1] |
| 147 | 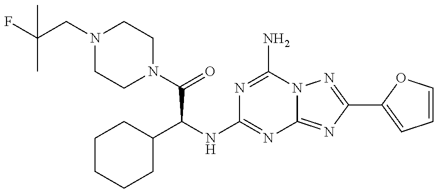 [SMILES: CC(C)(F)CN1CCN(C(=O)[C@@H](Nc2nc(N)n3nc(-c4ccco4)nc3n2)C2CCCCC2)CC1] |
| 148 | 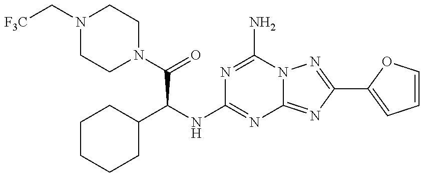 [SMILES: Nc1nc(N[C@H](C(=O)N2CCN(CC(F)(F)F)CC2)C2CCCCC2)nc2nc(-c3ccco3)nn12] |
| 149 | 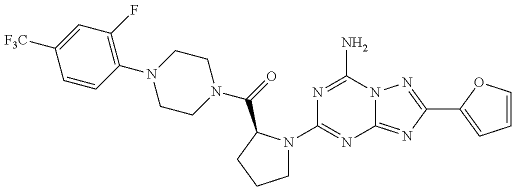 [SMILES: Nc1nc(N2CCC[C@H]2C(=O)N2CCN(c3ccc(C(F)(F)F)cc3F)CC2)nc2nc(-c3ccco3)nn12] |
| 150 | 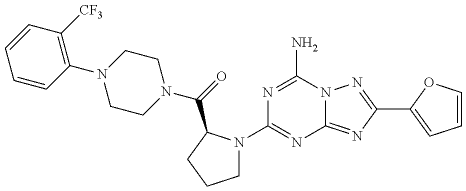 [SMILES: Nc1nc(N2CCC[C@H]2C(=O)N2CCN(c3ccccc3C(F)(F)F)CC2)nc2nc(-c3ccco3)nn12] |
| 151 | 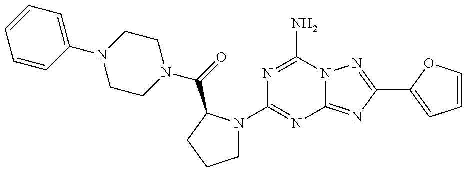 [SMILES: Nc1nc(N2CCC[C@H]2C(=O)N2CCN(c3ccccc3)CC2)nc2nc(-c3ccco3)nn12] |
| 152 | 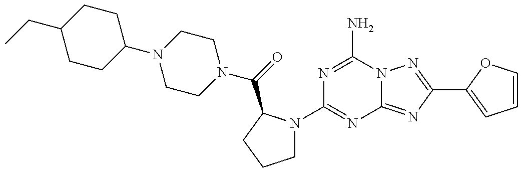 [SMILES: CCC1CCC(N2CCN(C(=O)[C@@H]3CCCN3c3nc(N)n4nc(-c5ccco5)nc4n3)CC2)CC1] |
| 153 | 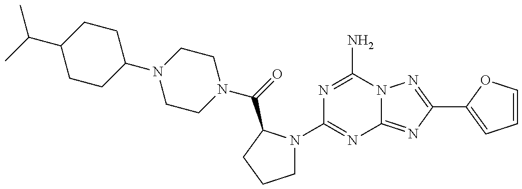 [SMILES: CC(C)C1CCC(N2CCN(C(=O)[C@@H]3CCCN3c3nc(N)n4nc(-c5ccco5)nc4n3)CC2)CC1] |
| 154 | 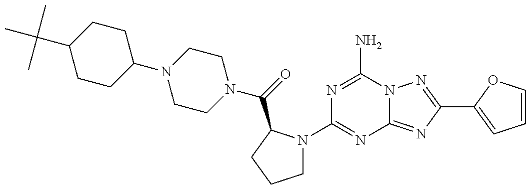 [SMILES: CC(C)(C)C1CCC(N2CCN(C(=O)[C@@H]3CCCN3c3nc(N)n4nc(-c5ccco5)nc4n3)CC2)CC1] |
| 155 | 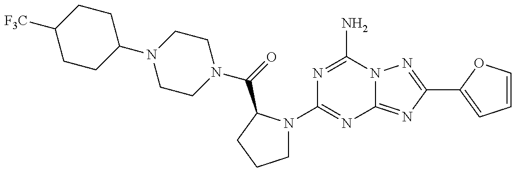 [SMILES: Nc1nc(N2CCC[C@H]2C(=O)N2CCN(C3CCC(C(F)(F)F)CC3)CC2)nc2nc(-c3ccco3)nn12] |
| 156 | 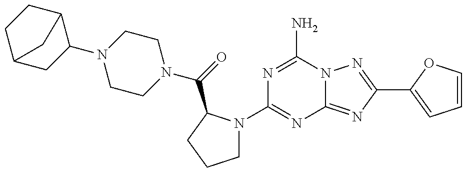 [SMILES: Nc1nc(N2CCC[C@H]2C(=O)N2CCN(C3CC4CCC3C4)CC2)nc2nc(-c3ccco3)nn12] |
| 157 | 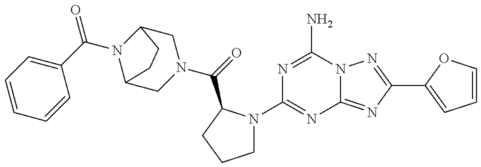 [SMILES: Nc1nc(N2CCC[C@H]2C(=O)N2CC3CCC(C2)N3C(=O)c2ccccc2)nc2nc(-c3ccco3)nn12] |
| 158 | 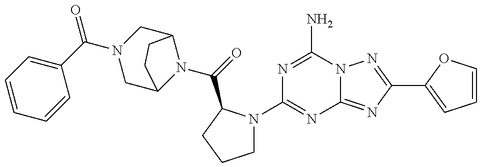 [SMILES: Nc1nc(N2CCC[C@H]2C(=O)N2C3CCC2CN(C(=O)c2ccccc2)C3)nc2nc(-c3ccco3)nn12] |
| 159 | 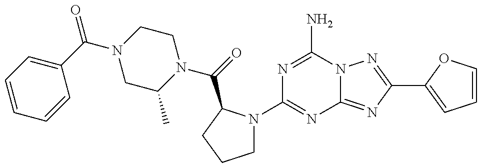 [SMILES: C[C@@H]1CN(C(=O)c2ccccc2)CCN1C(=O)[C@@H]1CCCN1c1nc(N)n2nc(-c3ccco3)nc2n1] |
| 160 | 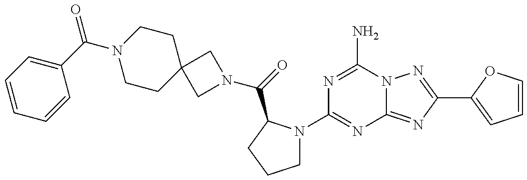 [SMILES: Nc1nc(N2CCC[C@H]2C(=O)N2CC3(CCN(C(=O)c4ccccc4)CC3)C2)nc2nc(-c3ccco3)nn12] |
| 161 | 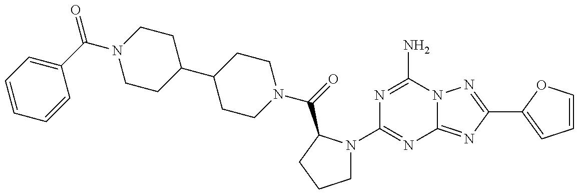 [SMILES: Nc1nc(N2CCC[C@H]2C(=O)N2CCC(C3CCN(C(=O)c4ccccc4)CC3)CC2)nc2nc(-c3ccco3)nn12] |
| 162 | 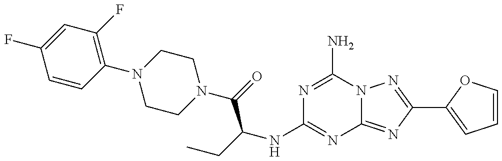 [SMILES: CC[C@H](Nc1nc(N)n2nc(-c3ccco3)nc2n1)C(=O)N1CCN(c2ccc(F)cc2F)CC1] |
| 163 | 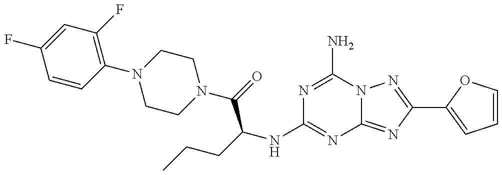 [SMILES: CCC[C@H](Nc1nc(N)n2nc(-c3ccco3)nc2n1)C(=O)N1CCN(c2ccc(F)cc2F)CC1] |
| 164 | 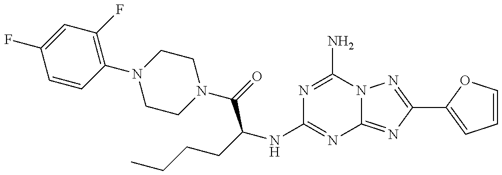 [SMILES: CCCC[C@H](Nc1nc(N)n2nc(-c3ccco3)nc2n1)C(=O)N1CCN(c2ccc(F)cc2F)CC1] |
| 165 | 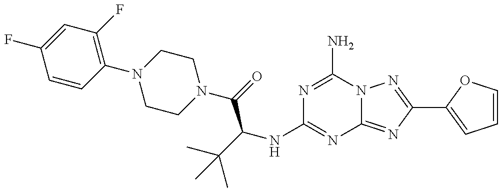 [SMILES: CC(C)(C)[C@H](Nc1nc(N)n2nc(-c3ccco3)nc2n1)C(=O)N1CCN(c2ccc(F)cc2F)CC1] |
| 166 | 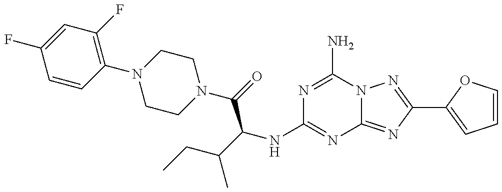 [SMILES: CCC(C)[C@H](Nc1nc(N)n2nc(-c3ccco3)nc2n1)C(=O)N1CCN(c2ccc(F)cc2F)CC1] |
| 167 | 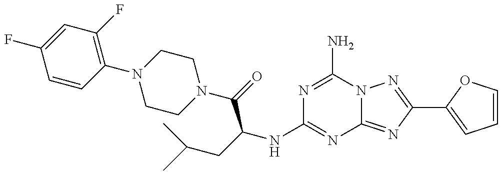 [SMILES: CC(C)C[C@H](Nc1nc(N)n2nc(-c3ccco3)nc2n1)C(=O)N1CCN(c2ccc(F)cc2F)CC1] |
| 168 | 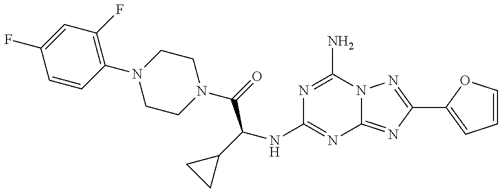 [SMILES: Nc1nc(N[C@H](C(=O)N2CCN(c3ccc(F)cc3F)CC2)C2CC2)nc2nc(-c3ccco3)nn12] |
| 169 | 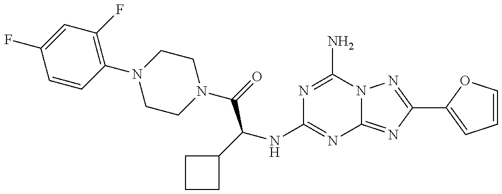 [SMILES: Nc1nc(N[C@H](C(=O)N2CCN(c3ccc(F)cc3F)CC2)C2CCC2)nc2nc(-c3ccco3)nn12] |
| 170 | 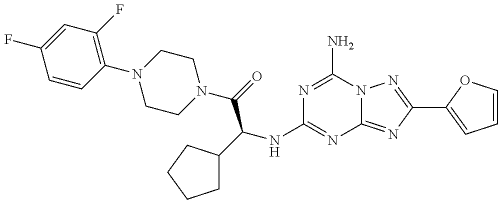 [SMILES: Nc1nc(N[C@H](C(=O)N2CCN(c3ccc(F)cc3F)CC2)C2CCCC2)nc2nc(-c3ccco3)nn12] |
| 171 | 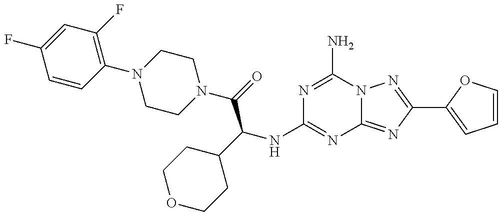 [SMILES: Nc1nc(N[C@H](C(=O)N2CCN(c3ccc(F)cc3F)CC2)C2CCOCC2)nc2nc(-c3ccco3)nn12] |
| 172 | 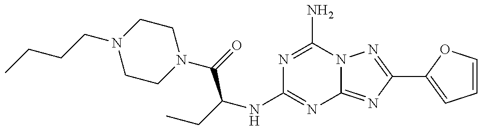 [SMILES: CCCCN1CCN(C(=O)[C@H](CC)Nc2nc(N)n3nc(-c4ccco4)nc3n2)CC1] |
| 173 | 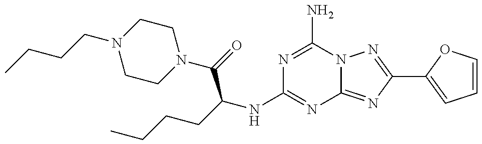 [SMILES: CCCC[C@H](Nc1nc(N)n2nc(-c3ccco3)nc2n1)C(=O)N1CCN(CCCC)CC1] |
| 174 | 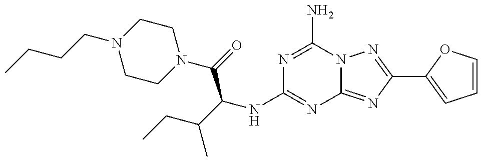 [SMILES: CCCCN1CCN(C(=O)[C@@H](Nc2nc(N)n3nc(-c4ccco4)nc3n2)C(C)CC)CC1] |
| 175 | 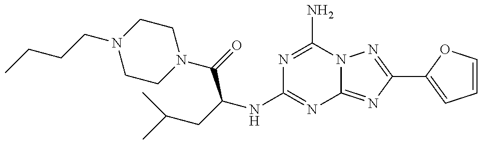 [SMILES: CCCCN1CCN(C(=O)[C@H](CC(C)C)Nc2nc(N)n3nc(-c4ccco4)nc3n2)CC1] |
| 176 | 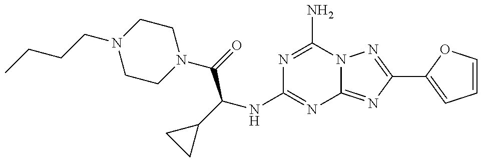 [SMILES: CCCCN1CCN(C(=O)[C@@H](Nc2nc(N)n3nc(-c4ccco4)nc3n2)C2CC2)CC1] |
| 177 | 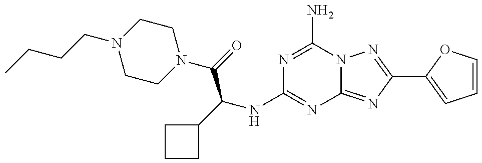 [SMILES: CCCCN1CCN(C(=O)[C@@H](Nc2nc(N)n3nc(-c4ccco4)nc3n2)C2CCC2)CC1] |
| 178 | 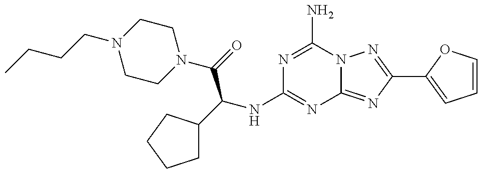 [SMILES: CCCCN1CCN(C(=O)[C@@H](Nc2nc(N)n3nc(-c4ccco4)nc3n2)C2CCCC2)CC1] |
| 179 | 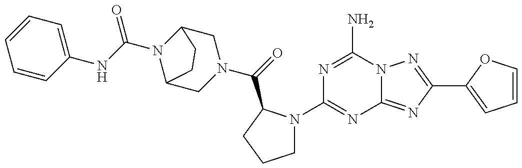 [SMILES: Nc1nc(N2CCC[C@H]2C(=O)N2CC3CCC(C2)N3C(=O)Nc2ccccc2)nc2nc(-c3ccco3)nn12] |
| 180 | 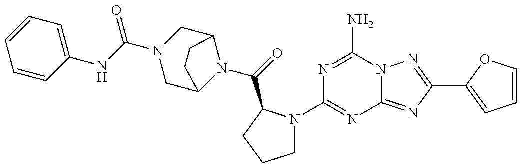 [SMILES: Nc1nc(N2CCC[C@H]2C(=O)N2C3CCC2CN(C(=O)Nc2ccccc2)C3)nc2nc(-c3ccco3)nn12] |
| 181 | 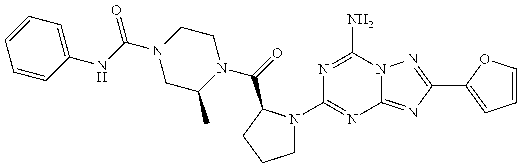 [SMILES: C[C@H]1CN(C(=O)Nc2ccccc2)CCN1C(=O)[C@@H]1CCCN1c1nc(N)n2nc(-c3ccco3)nc2n1] |
| 182 | 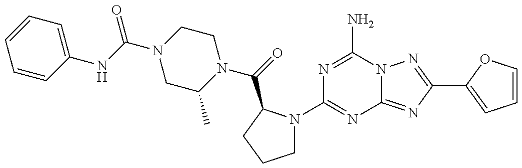 [SMILES: C[C@@H]1CN(C(=O)Nc2ccccc2)CCN1C(=O)[C@@H]1CCCN1c1nc(N)n2nc(-c3ccco3)nc2n1] |
| 183 | 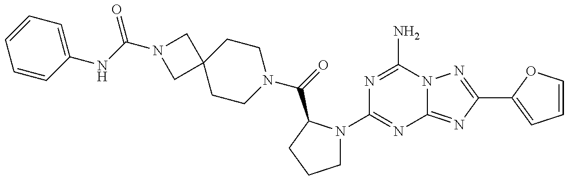 [SMILES: Nc1nc(N2CCC[C@H]2C(=O)N2CCC3(CC2)CN(C(=O)Nc2ccccc2)C3)nc2nc(-c3ccco3)nn12] |
| 184 | 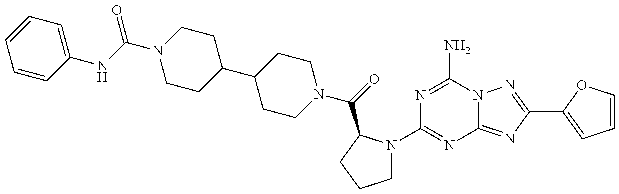 [SMILES: Nc1nc(N2CCC[C@H]2C(=O)N2CCC(C3CCN(C(=O)Nc4ccccc4)CC3)CC2)nc2nc(-c3ccco3)nn12] |
| 185 | 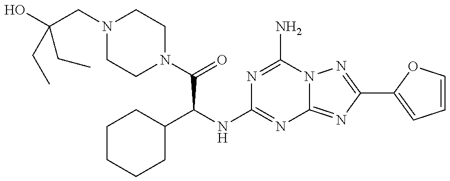 [SMILES: CCC(O)(CC)CN1CCN(C(=O)[C@@H](Nc2nc(N)n3nc(-c4ccco4)nc3n2)C2CCCCC2)CC1] |
| 186 | 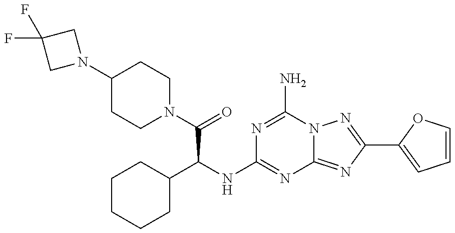 [SMILES: Nc1nc(N[C@H](C(=O)N2CCC(N3CC(F)(F)C3)CC2)C2CCCCC2)nc2nc(-c3ccco3)nn12] |
| 187 | 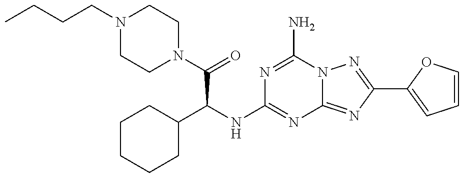 [SMILES: CCCCN1CCN(C(=O)[C@@H](Nc2nc(N)n3nc(-c4ccco4)nc3n2)C2CCCCC2)CC1] |
| 188 | 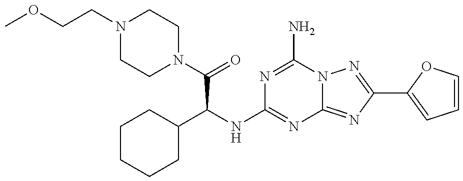 [SMILES: COCCN1CCN(C(=O)[C@@H](Nc2nc(N)n3nc(-c4ccco4)nc3n2)C2CCCCC2)CC1] |
| 189 | 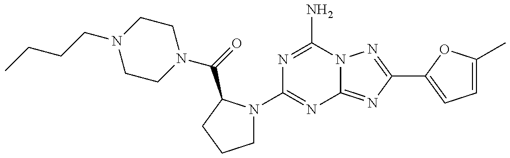 [SMILES: CCCCN1CCN(C(=O)[C@@H]2CCCN2c2nc(N)n3nc(-c4ccc(C)o4)nc3n2)CC1] |
| 190 | 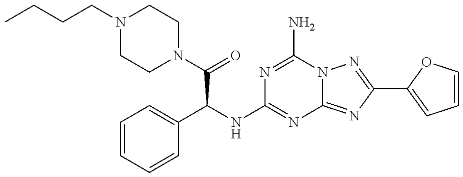 [SMILES: CCCCN1CCN(C(=O)[C@@H](Nc2nc(N)n3nc(-c4ccco4)nc3n2)c2ccccc2)CC1] |
| 191 | 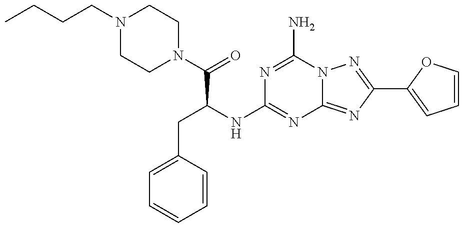 [SMILES: CCCCN1CCN(C(=O)[C@H](Cc2ccccc2)Nc2nc(N)n3nc(-c4ccco4)nc3n2)CC1] |
| 192 | 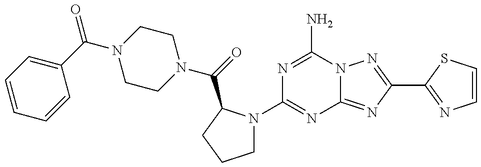 [SMILES: Nc1nc(N2CCC[C@H]2C(=O)N2CCN(C(=O)c3ccccc3)CC2)nc2nc(-c3nccs3)nn12] |
| 193 | 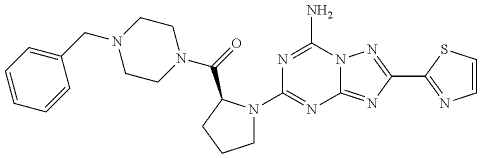 [SMILES: Nc1nc(N2CCC[C@H]2C(=O)N2CCN(Cc3ccccc3)CC2)nc2nc(-c3nccs3)nn12] |
| 194 | 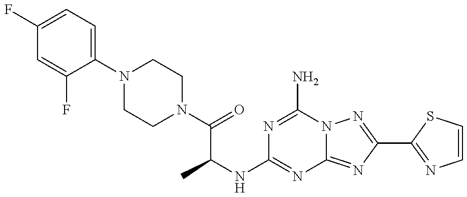 [SMILES: C[C@H](Nc1nc(N)n2nc(-c3nccs3)nc2n1)C(=O)N1CCN(c2ccc(F)cc2F)CC1] |
| 195 | 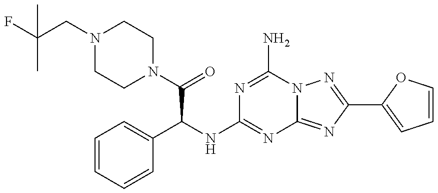 [SMILES: CC(C)(F)CN1CCN(C(=O)[C@@H](Nc2nc(N)n3nc(-c4ccco4)nc3n2)c2ccccc2)CC1] |
| 196 | 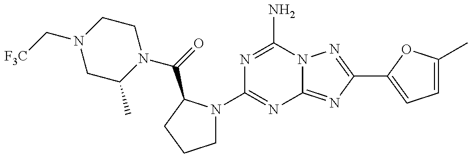 [SMILES: Cc1ccc(-c2nc3nc(N4CCC[C@H]4C(=O)N4CCN(CC(F)(F)F)C[C@H]4C)nc(N)n3n2)o1] |
| 197 | 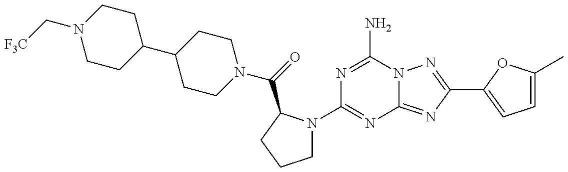 [SMILES: Cc1ccc(-c2nc3nc(N4CCC[C@H]4C(=O)N4CCC(C5CCN(CC(F)(F)F)CC5)CC4)nc(N)n3n2)o1] |
| 198 | 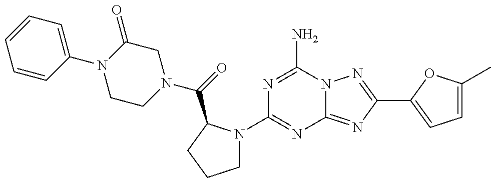 [SMILES: Cc1ccc(-c2nc3nc(N4CCC[C@H]4C(=O)N4CCN(c5ccccc5)C(=O)C4)nc(N)n3n2)o1] |
| 199 | 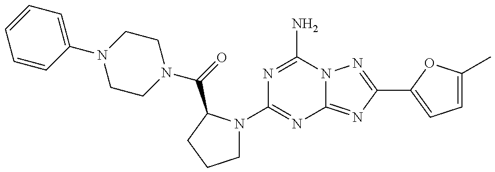 [SMILES: Cc1ccc(-c2nc3nc(N4CCC[C@H]4C(=O)N4CCN(c5ccccc5)CC4)nc(N)n3n2)o1] |
| 200 | 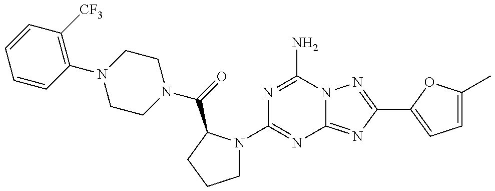 [SMILES: Cc1ccc(-c2nc3nc(N4CCC[C@H]4C(=O)N4CCN(c5ccccc5C(F)(F)F)CC4)nc(N)n3n2)o1] |
| 201 | 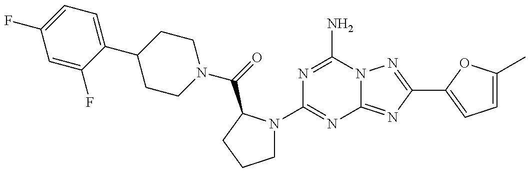 [SMILES: Cc1ccc(-c2nc3nc(N4CCC[C@H]4C(=O)N4CCC(c5ccc(F)cc5F)CC4)nc(N)n3n2)o1] |
| 202 | 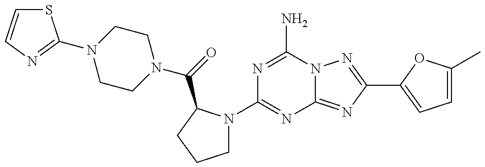 [SMILES: Cc1ccc(-c2nc3nc(N4CCC[C@H]4C(=O)N4CCN(c5nccs5)CC4)nc(N)n3n2)o1] |
| 203 | 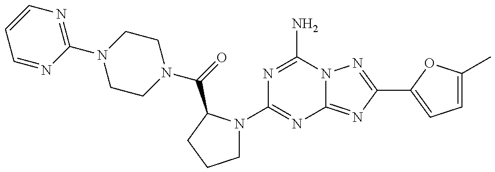 [SMILES: Cc1ccc(-c2nc3nc(N4CCC[C@H]4C(=O)N4CCN(c5ncccn5)CC4)nc(N)n3n2)o1] |
| 204 | 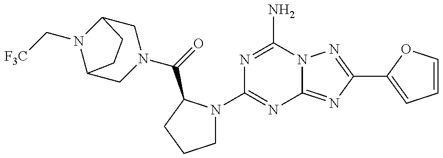 [SMILES: Nc1nc(N2CCC[C@H]2C(=O)N2CC3CCC(C2)N3CC(F)(F)F)nc2nc(-c3ccco3)nn12] |
| 205 | 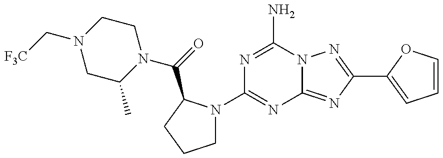 [SMILES: C[C@@H]1CN(CC(F)(F)F)CCN1C(=O)[C@@H]1CCCN1c1nc(N)n2nc(-c3ccco3)nc2n1] |
| 206 | 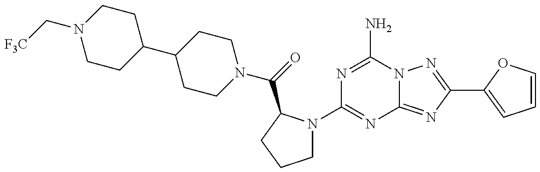 [SMILES: Nc1nc(N2CCC[C@H]2C(=O)N2CCC(C3CCN(CC(F)(F)F)CC3)CC2)nc2nc(-c3ccco3)nn12] |
| 207 | 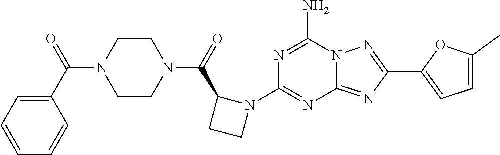 [SMILES: Cc1ccc(-c2nc3nc(N4CC[C@H]4C(=O)N4CCN(C(=O)c5ccccc5)CC4)nc(N)n3n2)o1] |
| 208 | 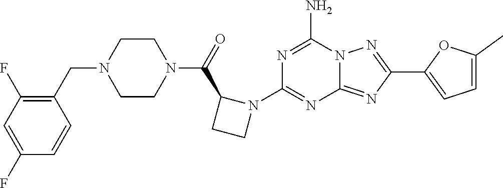 [SMILES: Cc1ccc(-c2nc3nc(N4CC[C@H]4C(=O)N4CCN(Cc5ccc(F)cc5F)CC4)nc(N)n3n2)o1] |
| 209 | 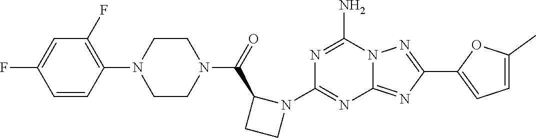 [SMILES: Cc1ccc(-c2nc3nc(N4CC[C@H]4C(=O)N4CCN(c5ccc(F)cc5F)CC4)nc(N)n3n2)o1] |
| 210 | 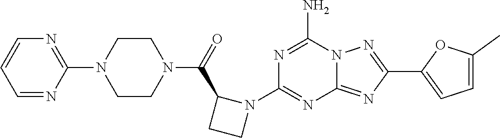 [SMILES: Cc1ccc(-c2nc3nc(N4CC[C@H]4C(=O)N4CCN(c5ncccn5)CC4)nc(N)n3n2)o1] |
| 211 | 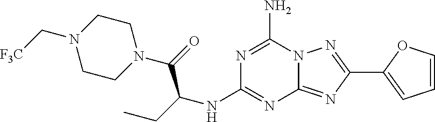 [SMILES: CC[C@H](Nc1nc(N)n2nc(-c3ccco3)nc2n1)C(=O)N1CCN(CC(F)(F)F)CC1] |
| 212 | 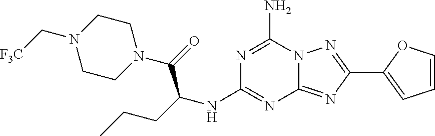 [SMILES: CCC[C@H](Nc1nc(N)n2nc(-c3ccco3)nc2n1)C(=O)N1CCN(CC(F)(F)F)CC1] |
| 213 | 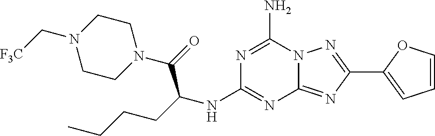 [SMILES: CCCC[C@H](Nc1nc(N)n2nc(-c3ccco3)nc2n1)C(=O)N1CCN(CC(F)(F)F)CC1] |
| 214 | 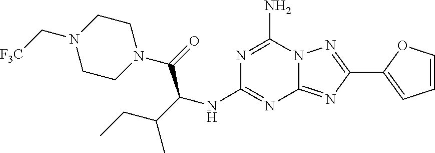 [SMILES: CCC(C)[C@H](Nc1nc(N)n2nc(-c3ccco3)nc2n1)C(=O)N1CCN(CC(F)(F)F)CC1] |
| 215 | 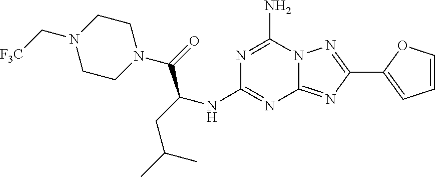 [SMILES: CC(C)C[C@H](Nc1nc(N)n2nc(-c3ccco3)nc2n1)C(=O)N1CCN(CC(F)(F)F)CC1] |
| 216 | 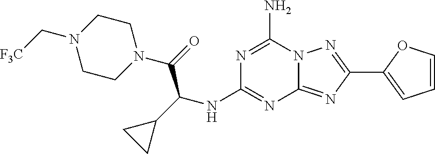 [SMILES: Nc1nc(N[C@H](C(=O)N2CCN(CC(F)(F)F)CC2)C2CC2)nc2nc(-c3ccco3)nn12] |
| 217 | 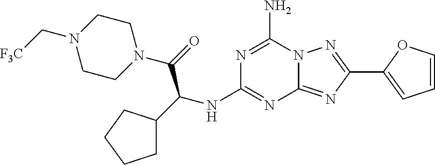 [SMILES: Nc1nc(N[C@H](C(=O)N2CCN(CC(F)(F)F)CC2)C2CCCC2)nc2nc(-c3ccco3)nn12] |
| 218 | 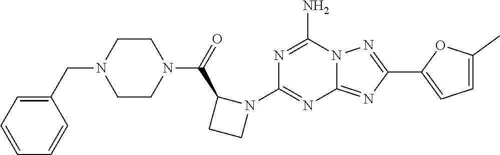 [SMILES: Cc1ccc(-c2nc3nc(N4CC[C@H]4C(=O)N4CCN(Cc5ccccc5)CC4)nc(N)n3n2)o1] |
| 219 | 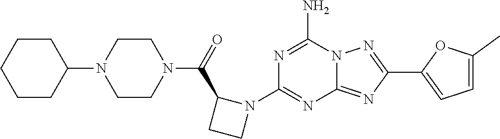 [SMILES: Cc1ccc(-c2nc3nc(N4CC[C@H]4C(=O)N4CCN(C5CCCCC5)CC4)nc(N)n3n2)o1] |
| 220 | 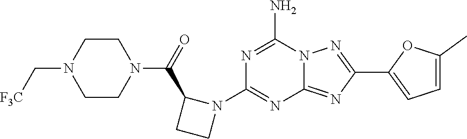 [SMILES: Cc1ccc(-c2nc3nc(N4CC[C@H]4C(=O)N4CCN(CC(F)(F)F)CC4)nc(N)n3n2)o1] |
| 221 | 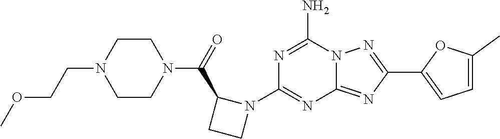 |
| 222 | 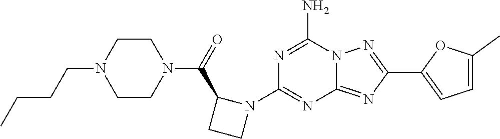 [SMILES: CCCCN1CCN(C(=O)[C@@H]2CCN2c2nc(N)n3nc(-c4ccc(C)o4)nc3n2)CC1] |
| 223 | 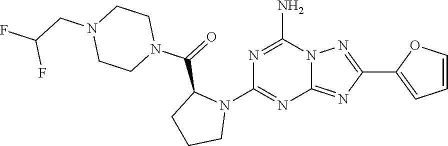 [SMILES: Nc1nc(N2CCC[C@H]2C(=O)N2CCN(CC(F)F)CC2)nc2nc(-c3ccco3)nn12] |
| 224 | 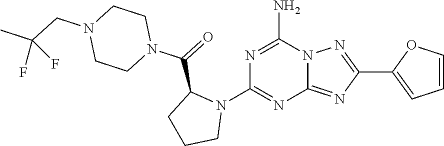 [SMILES: CC(F)(F)CN1CCN(C(=O)[C@@H]2CCCN2c2nc(N)n3nc(-c4ccco4)nc3n2)CC1] |
| 225 | 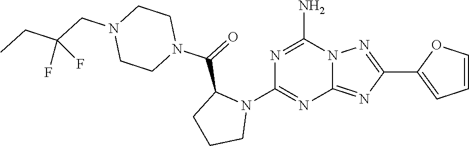 [SMILES: CCC(F)(F)CN1CCN(C(=O)[C@@H]2CCCN2c2nc(N)n3nc(-c4ccco4)nc3n2)CC1] |
| 226 | 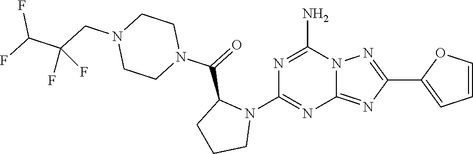 [SMILES: Nc1nc(N2CCC[C@H]2C(=O)N2CCN(CC(F)(F)C(F)F)CC2)nc2nc(-c3ccco3)nn12] |
| 227 | 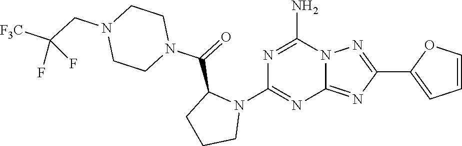 [SMILES: Nc1nc(N2CCC[C@H]2C(=O)N2CCN(CC(F)(F)C(F)(F)F)CC2)nc2nc(-c3ccco3)nn12] |
| 228 | 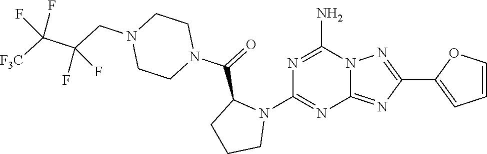 [SMILES: Nc1nc(N2CCC[C@H]2C(=O)N2CCN(CC(F)(F)C(F)(F)C(F)(F)F)CC2)nc2nc(-c3ccco3)nn12] |
| 229 | 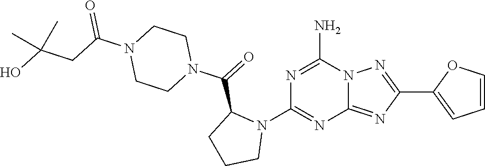 [SMILES: CC(C)(O)CC(=O)N1CCN(C(=O)[C@@H]2CCCN2c2nc(N)n3nc(-c4ccco4)nc3n2)CC1] |
| 230 | 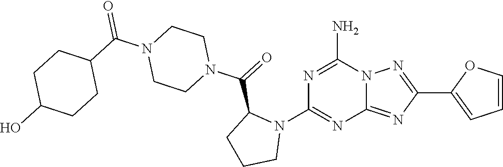 [SMILES: Nc1nc(N2CCC[C@H]2C(=O)N2CCN(C(=O)C3CCC(O)CC3)CC2)nc2nc(-c3ccco3)nn12] |
| 231 | 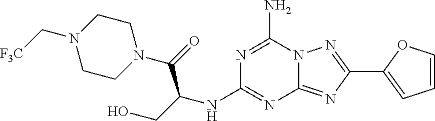 [SMILES: Nc1nc(N[C@@H](CO)C(=O)N2CCN(CC(F)(F)F)CC2)nc2nc(-c3ccco3)nn12] |
| 232 | 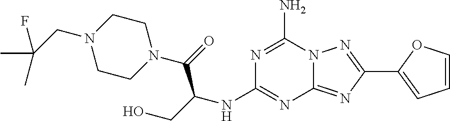 [SMILES: CC(C)(F)CN1CCN(C(=O)[C@H](CO)Nc2nc(N)n3nc(-c4ccco4)nc3n2)CC1] |
| 233 | 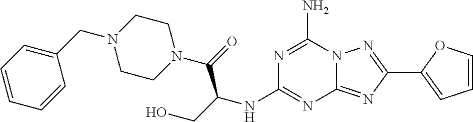 [SMILES: Nc1nc(N[C@@H](CO)C(=O)N2CCN(Cc3ccccc3)CC2)nc2nc(-c3ccco3)nn12] |
| 234 | 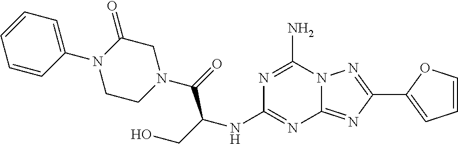 [SMILES: Nc1nc(N[C@@H](CO)C(=O)N2CCN(c3ccccc3)C(=O)C2)nc2nc(-c3ccco3)nn12] |
| 235 | 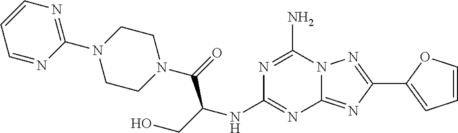 [SMILES: Nc1nc(N[C@@H](CO)C(=O)N2CCN(c3ncccn3)CC2)nc2nc(-c3ccco3)nn12] |
| 236 | 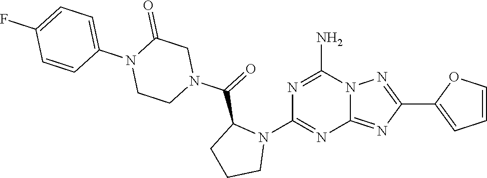 [SMILES: Nc1nc(N2CCC[C@H]2C(=O)N2CCN(c3ccc(F)cc3)C(=O)C2)nc2nc(-c3ccco3)nn12] |
| 237 | 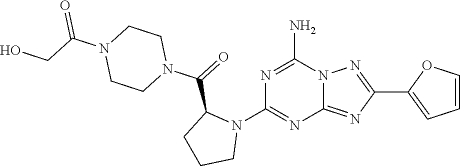 [SMILES: Nc1nc(N2CCC[C@H]2C(=O)N2CCN(C(=O)CO)CC2)nc2nc(-c3ccco3)nn12] |
| 238 | 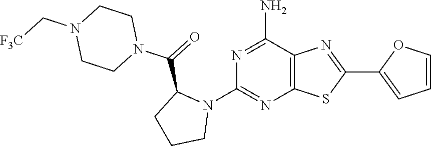 [SMILES: Nc1nc(N2CCC[C@H]2C(=O)N2CCN(CC(F)(F)F)CC2)nc2sc(-c3ccco3)nc12] |
| 239 | 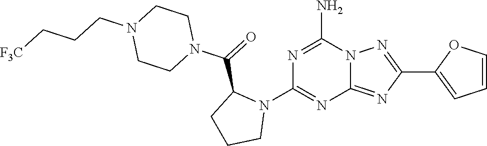 [SMILES: Nc1nc(N2CCC[C@H]2C(=O)N2CCN(CCCC(F)(F)F)CC2)nc2nc(-c3ccco3)nn12] |
| 240 | 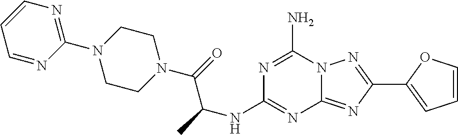 [SMILES: C[C@H](Nc1nc(N)n2nc(-c3ccco3)nc2n1)C(=O)N1CCN(c2ncccn2)CC1] |
| 241 | 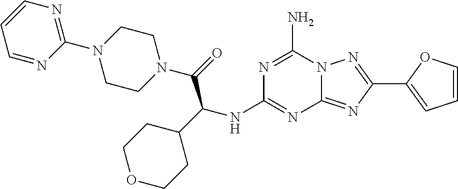 [SMILES: Nc1nc(N[C@H](C(=O)N2CCN(c3ncccn3)CC2)C2CCOCC2)nc2nc(-c3ccco3)nn12] |
| 242 | 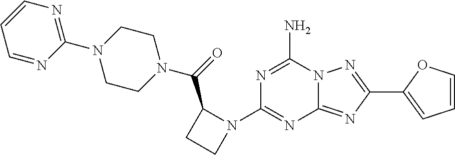 [SMILES: Nc1nc(N2CC[C@H]2C(=O)N2CCN(c3ncccn3)CC2)nc2nc(-c3ccco3)nn12] |
| 243 | 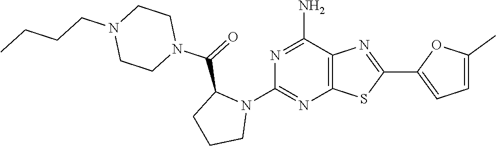 [SMILES: CCCCN1CCN(C(=O)[C@@H]2CCCN2c2nc(N)c3nc(-c4ccc(C)o4)sc3n2)CC1] |
| 244 | 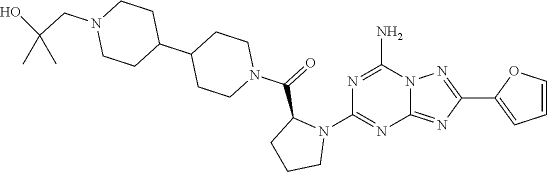 [SMILES: CC(C)(O)CN1CCC(C2CCN(C(=O)[C@@H]3CCCN3c3nc(N)n4nc(-c5ccco5)nc4n3)CC2)CC1] |
| 245 | 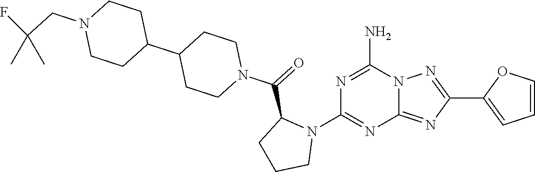 [SMILES: CC(C)(F)CN1CCC(C2CCN(C(=O)[C@@H]3CCCN3c3nc(N)n4nc(-c5ccco5)nc4n3)CC2)CC1] |
| 246 | 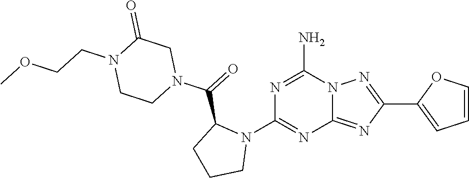 [SMILES: COCCN1CCN(C(=O)[C@@H]2CCCN2c2nc(N)n3nc(-c4ccco4)nc3n2)CC1=O] |
| 247 | 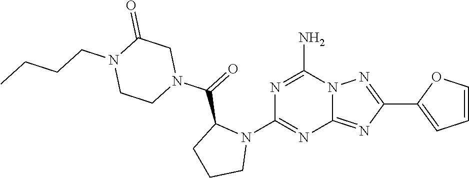 [SMILES: CCCCN1CCN(C(=O)[C@@H]2CCCN2c2nc(N)n3nc(-c4ccco4)nc3n2)CC1=O] |
| 248 | 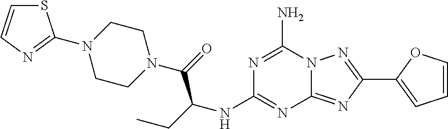 [SMILES: CC[C@H](Nc1nc(N)n2nc(-c3ccco3)nc2n1)C(=O)N1CCN(c2nccs2)CC1] |
| 249 | 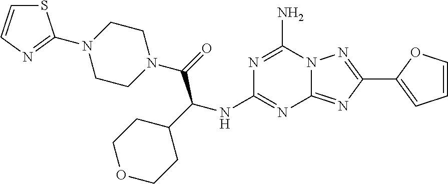 [SMILES: Nc1nc(N[C@H](C(=O)N2CCN(c3nccs3)CC2)C2CCOCC2)nc2nc(-c3ccco3)nn12] |
| 250 | 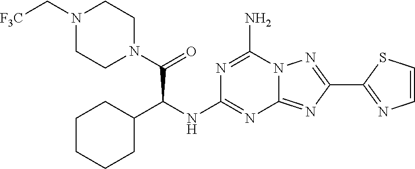 [SMILES: Nc1nc(N[C@H](C(=O)N2CCN(CC(F)(F)F)CC2)C2CCCCC2)nc2nc(-c3nccs3)nn12] |
| 251 | 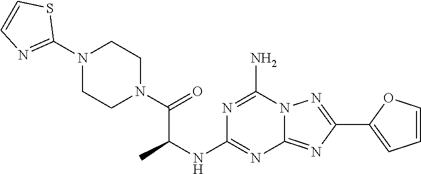 [SMILES: C[C@H](Nc1nc(N)n2nc(-c3ccco3)nc2n1)C(=O)N1CCN(c2nccs2)CC1] |
| 252 | 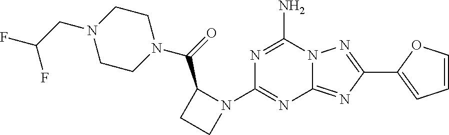 [SMILES: Nc1nc(N2CC[C@H]2C(=O)N2CCN(CC(F)F)CC2)nc2nc(-c3ccco3)nn12] |
| 253 | 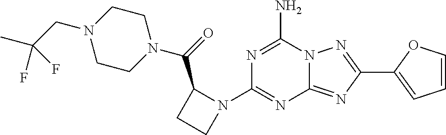 [SMILES: CC(F)(F)CN1CCN(C(=O)[C@@H]2CCN2c2nc(N)n3nc(-c4ccco4)nc3n2)CC1] |
| 254 | 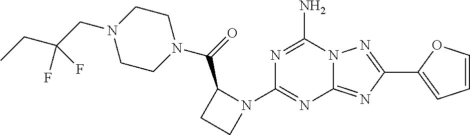 [SMILES: CCC(F)(F)CN1CCN(C(=O)[C@@H]2CCN2c2nc(N)n3nc(-c4ccco4)nc3n2)CC1] |
| 255 | 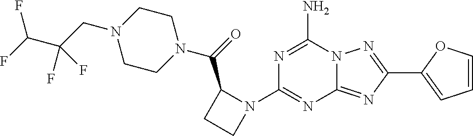 [SMILES: Nc1nc(N2CC[C@H]2C(=O)N2CCN(CC(F)(F)C(F)F)CC2)nc2nc(-c3ccco3)nn12] |
| 256 | 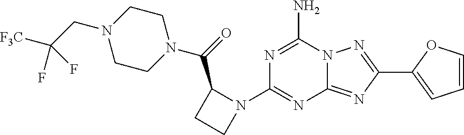 [SMILES: Nc1nc(N2CC[C@H]2C(=O)N2CCN(CC(F)(F)C(F)(F)F)CC2)nc2nc(-c3ccco3)nn12] |
| 257 | 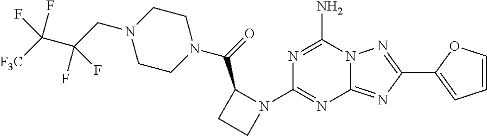 [SMILES: Nc1nc(N2CC[C@H]2C(=O)N2CCN(CC(F)(F)C(F)(F)C(F)(F)F)CC2)nc2nc(-c3ccco3)nn12] |
| 258 | 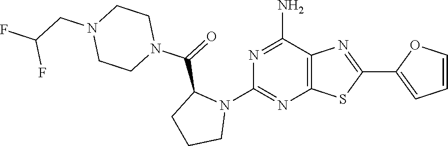 [SMILES: Nc1nc(N2CCC[C@H]2C(=O)N2CCN(CC(F)F)CC2)nc2sc(-c3ccco3)nc12] |
| 259 | 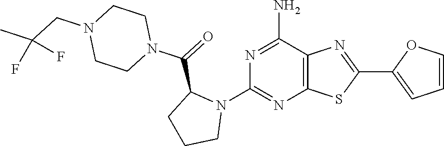 [SMILES: CC(F)(F)CN1CCN(C(=O)[C@@H]2CCCN2c2nc(N)c3nc(-c4ccco4)sc3n2)CC1] |
| 260 | 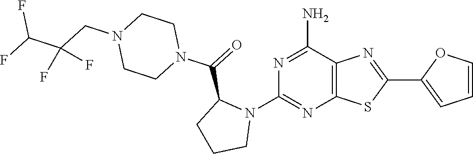 [SMILES: Nc1nc(N2CCC[C@H]2C(=O)N2CCN(CC(F)(F)C(F)F)CC2)nc2sc(-c3ccco3)nc12] |
| 261 | 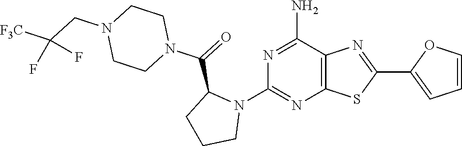 [SMILES: Nc1nc(N2CCC[C@H]2C(=O)N2CCN(CC(F)(F)C(F)(F)F)CC2)nc2sc(-c3ccco3)nc12] |
| 262 | 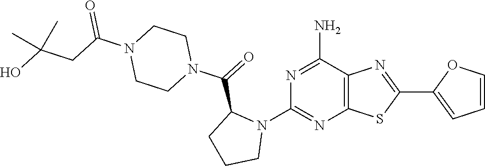 [SMILES: CC(C)(O)CC(=O)N1CCN(C(=O)[C@@H]2CCCN2c2nc(N)c3nc(-c4ccco4)sc3n2)CC1] |
| 263 | 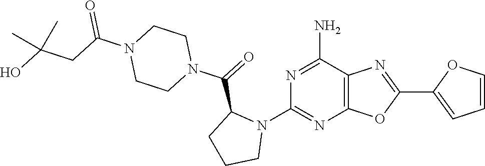 [SMILES: CC(C)(O)CC(=O)N1CCN(C(=O)[C@@H]2CCCN2c2nc(N)c3nc(-c4ccco4)oc3n2)CC1] |
| 264 | 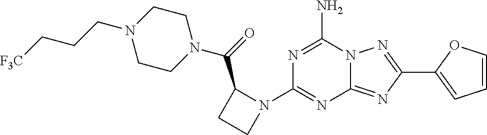 [SMILES: Nc1nc(N2CC[C@H]2C(=O)N2CCN(CCCC(F)(F)F)CC2)nc2nc(-c3ccco3)nn12] |
| 265 | 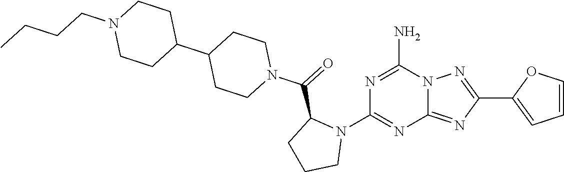 [SMILES: CCCCN1CCC(C2CCN(C(=O)[C@@H]3CCCN3c3nc(N)n4nc(-c5ccco5)nc4n3)CC2)CC1] |
| 266 | 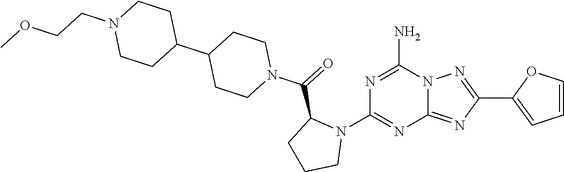 [SMILES: COCCN1CCC(C2CCN(C(=O)[C@@H]3CCCN3c3nc(N)n4nc(-c5ccco5)nc4n3)CC2)CC1] |
| 267 | 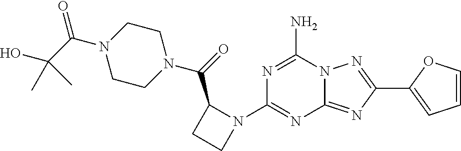 [SMILES: CC(C)(O)C(=O)N1CCN(C(=O)[C@@H]2CCN2c2nc(N)n3nc(-c4ccco4)nc3n2)CC1] |
| 268 | 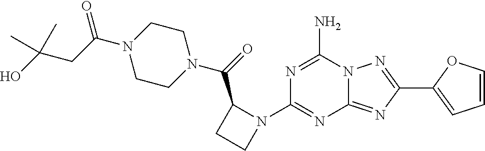 [SMILES: CC(C)(O)CC(=O)N1CCN(C(=O)[C@@H]2CCN2c2nc(N)n3nc(-c4ccco4)nc3n2)CC1] |
| 269 | 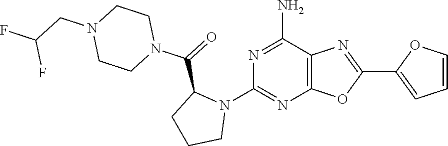 [SMILES: Nc1nc(N2CCC[C@H]2C(=O)N2CCN(CC(F)F)CC2)nc2oc(-c3ccco3)nc12] |
| 270 | 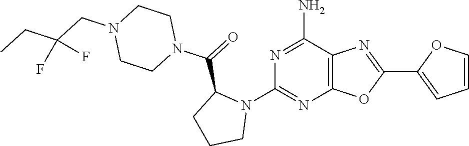 [SMILES: CCC(F)(F)CN1CCN(C(=O)[C@@H]2CCCN2c2nc(N)c3nc(-c4ccco4)oc3n2)CC1] |
| 271 | 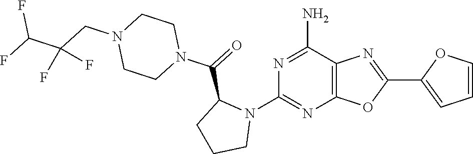 [SMILES: Nc1nc(N2CCC[C@H]2C(=O)N2CCN(CC(F)(F)C(F)F)CC2)nc2oc(-c3ccco3)nc12] |
| 272 | 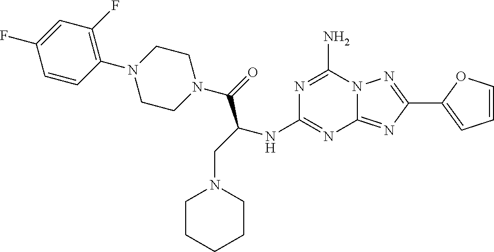 [SMILES: Nc1nc(N[C@@H](CN2CCCCC2)C(=O)N2CCN(c3ccc(F)cc3F)CC2)nc2nc(-c3ccco3)nn12] |
| 273 | 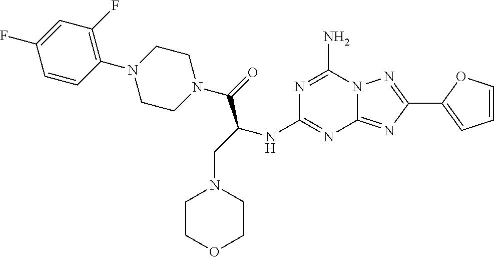 [SMILES: Nc1nc(N[C@@H](CN2CCOCC2)C(=O)N2CCN(c3ccc(F)cc3F)CC2)nc2nc(-c3ccco3)nn12] |
| 274 | 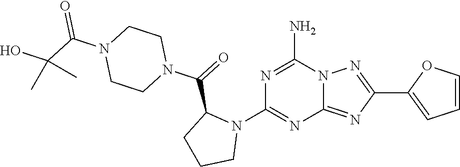 [SMILES: CC(C)(O)C(=O)N1CCN(C(=O)[C@@H]2CCCN2c2nc(N)n3nc(-c4ccco4)nc3n2)CC1] |
| 275 | 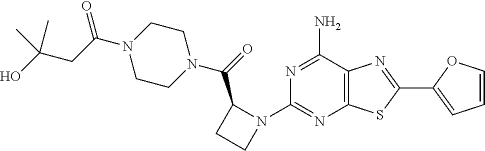 [SMILES: CC(C)(O)CC(=O)N1CCN(C(=O)[C@@H]2CCN2c2nc(N)c3nc(-c4ccco4)sc3n2)CC1] |
| 276 | 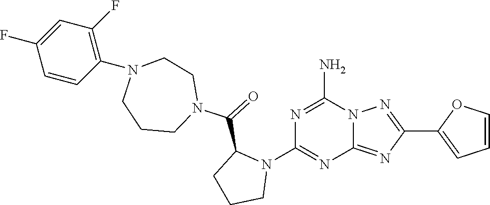 [SMILES: Nc1nc(N2CCC[C@H]2C(=O)N2CCCN(c3ccc(F)cc3F)CC2)nc2nc(-c3ccco3)nn12] |
| 277 | 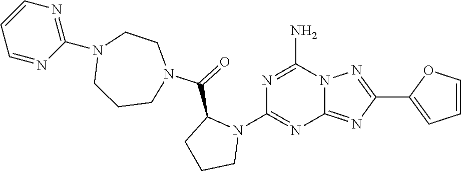 [SMILES: Nc1nc(N2CCC[C@H]2C(=O)N2CCCN(c3ncccn3)CC2)nc2nc(-c3ccco3)nn12] |
| 278 | 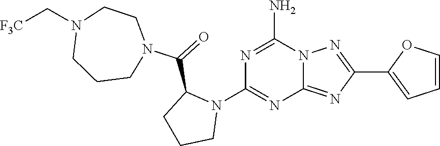 [SMILES: Nc1nc(N2CCC[C@H]2C(=O)N2CCCN(CC(F)(F)F)CC2)nc2nc(-c3ccco3)nn12] |
| 279 | 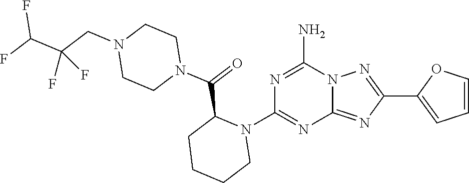 [SMILES: Nc1nc(N2CCCC[C@H]2C(=O)N2CCN(CC(F)(F)C(F)F)CC2)nc2nc(-c3ccco3)nn12] |
| 280 | 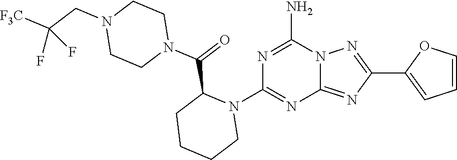 [SMILES: Nc1nc(N2CCCC[C@H]2C(=O)N2CCN(CC(F)(F)C(F)(F)F)CC2)nc2nc(-c3ccco3)nn12] |
| 281 | 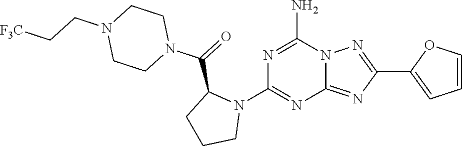 [SMILES: Nc1nc(N2CCC[C@H]2C(=O)N2CCN(CCC(F)(F)F)CC2)nc2nc(-c3ccco3)nn12] |
| 282 | 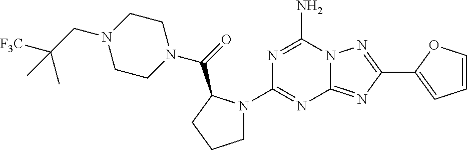 [SMILES: CC(C)(CN1CCN(C(=O)[C@@H]2CCCN2c2nc(N)n3nc(-c4ccco4)nc3n2)CC1)C(F)(F)F] |
| 283 | 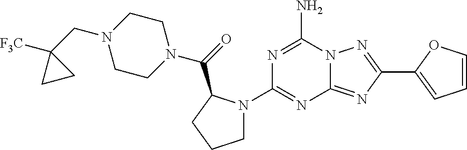 [SMILES: Nc1nc(N2CCC[C@H]2C(=O)N2CCN(CC3(C(F)(F)F)CC3)CC2)nc2nc(-c3ccco3)nn12] |
| 284 | 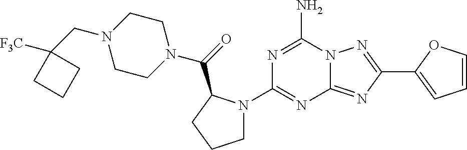 [SMILES: Nc1nc(N2CCC[C@H]2C(=O)N2CCN(CC3(C(F)(F)F)CCC3)CC2)nc2nc(-c3ccco3)nn12] |
| 285 | 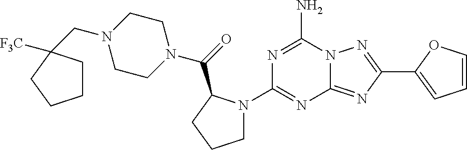 [SMILES: Nc1nc(N2CCC[C@H]2C(=O)N2CCN(CC3(C(F)(F)F)CCCC3)CC2)nc2nc(-c3ccco3)nn12] |
| 286 | 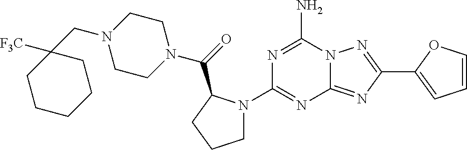 [SMILES: Nc1nc(N2CCC[C@H]2C(=O)N2CCN(CC3(C(F)(F)F)CCCCC3)CC2)nc2nc(-c3ccco3)nn12] |
| 287 | 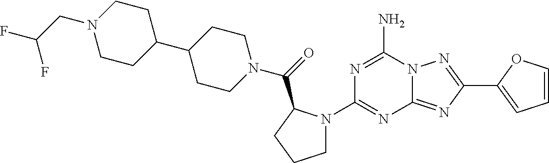 [SMILES: Nc1nc(N2CCC[C@H]2C(=O)N2CCC(C3CCN(CC(F)F)CC3)CC2)nc2nc(-c3ccco3)nn12] |
| 288 | 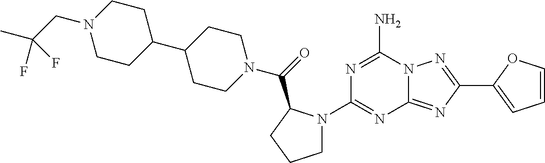 [SMILES: CC(F)(F)CN1CCC(C2CCN(C(=O)[C@@H]3CCCN3c3nc(N)n4nc(-c5ccco5)nc4n3)CC2)CC1] |
| 289 | 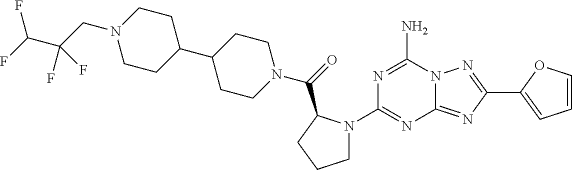 [SMILES: Nc1nc(N2CCC[C@H]2C(=O)N2CCC(C3CCN(CC(F)(F)C(F)F)CC3)CC2)nc2nc(-c3ccco3)nn12] |
| 290 | 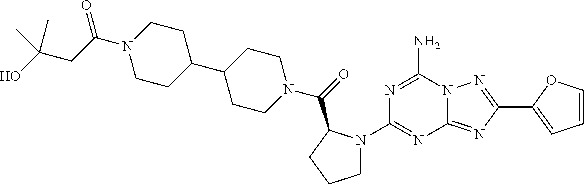 [SMILES: CC(C)(O)CC(=O)N1CCC(C2CCN(C(=O)[C@@H]3CCCN3c3nc(N)n4nc(-c5ccco5)nc4n3)CC2)CC1] |
| 291 | 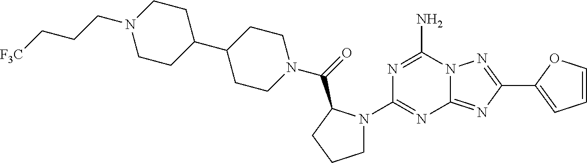 [SMILES: Nc1nc(N2CCC[C@H]2C(=O)N2CCC(C3CCN(CCCC(F)(F)F)CC3)CC2)nc2nc(-c3ccco3)nn12] |
| 292 | 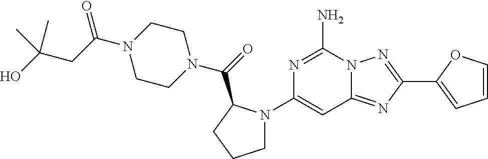 [SMILES: CC(C)(O)CC(=O)N1CCN(C(=O)[C@@H]2CCCN2c2cc3nc(-c4ccco4)nn3c(N)n2)CC1] |
| 293 | 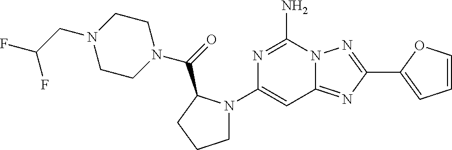 [SMILES: Nc1nc(N2CCC[C@H]2C(=O)N2CCN(CC(F)F)CC2)cc2nc(-c3ccco3)nn12] |
| 294 | 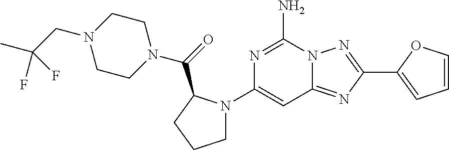 [SMILES: CC(F)(F)CN1CCN(C(=O)[C@@H]2CCCN2c2cc3nc(-c4ccco4)nn3c(N)n2)CC1] |
| 295 | 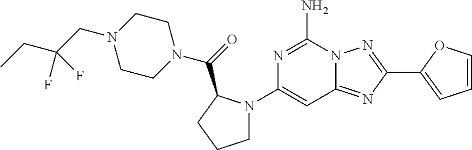 [SMILES: CCC(F)(F)CN1CCN(C(=O)[C@@H]2CCCN2c2cc3nc(-c4ccco4)nn3c(N)n2)CC1] |
| 296 | 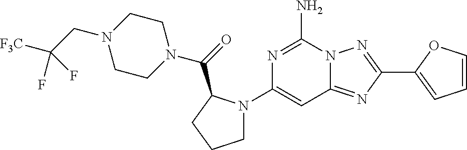 [SMILES: Nc1nc(N2CCC[C@H]2C(=O)N2CCN(CC(F)(F)C(F)(F)F)CC2)cc2nc(-c3ccco3)nn12] |
| 297 | 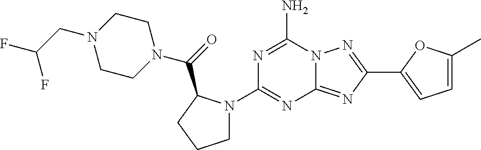 [SMILES: Cc1ccc(-c2nc3nc(N4CCC[C@H]4C(=O)N4CCN(CC(F)F)CC4)nc(N)n3n2)o1] |
| 298 | 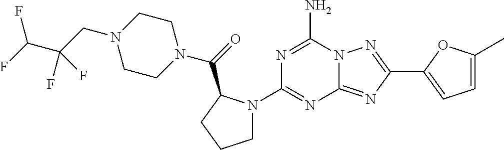 [SMILES: Cc1ccc(-c2nc3nc(N4CCC[C@H]4C(=O)N4CCN(CC(F)(F)C(F)F)CC4)nc(N)n3n2)o1] |
| 299 | 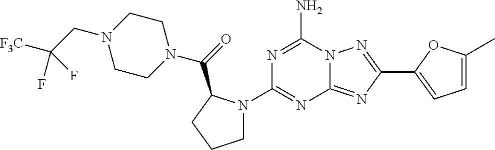 [SMILES: Cc1ccc(-c2nc3nc(N4CCC[C@H]4C(=O)N4CCN(CC(F)(F)C(F)(F)F)CC4)nc(N)n3n2)o1] |
| 300 | 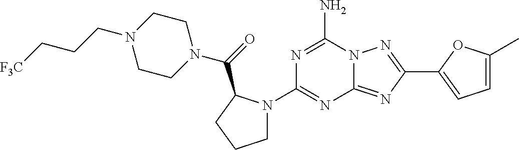 [SMILES: Cc1ccc(-c2nc3nc(N4CCC[C@H]4C(=O)N4CCN(CCCC(F)(F)F)CC4)nc(N)n3n2)o1] |
| 301 | 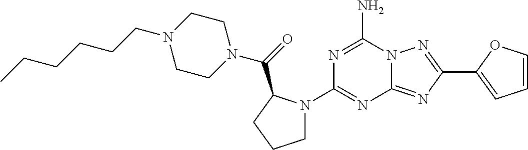 [SMILES: CCCCCCN1CCN(C(=O)[C@@H]2CCCN2c2nc(N)n3nc(-c4ccco4)nc3n2)CC1] |
| 302 | 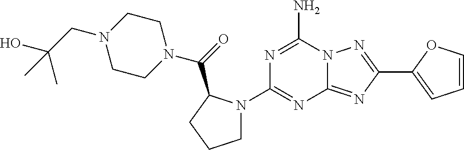 [SMILES: CC(C)(O)CN1CCN(C(=O)[C@@H]2CCCN2c2nc(N)n3nc(-c4ccco4)nc3n2)CC1] |
| 303 | 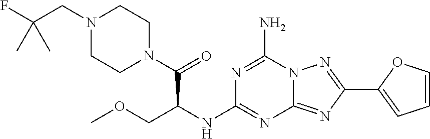 [SMILES: COC[C@H](Nc1nc(N)n2nc(-c3ccco3)nc2n1)C(=O)N1CCN(CC(C)(C)F)CC1] |
| 304 | 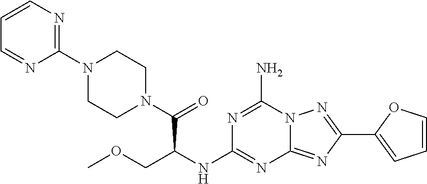 [SMILES: COC[C@H](Nc1nc(N)n2nc(-c3ccco3)nc2n1)C(=O)N1CCN(c2ncccn2)CC1] |
| 305 | 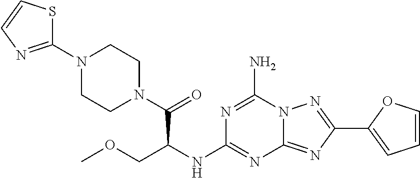 [SMILES: COC[C@H](Nc1nc(N)n2nc(-c3ccco3)nc2n1)C(=O)N1CCN(c2nccs2)CC1] |
| 306 | 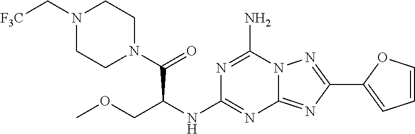 [SMILES: COC[C@H](Nc1nc(N)n2nc(-c3ccco3)nc2n1)C(=O)N1CCN(CC(F)(F)F)CC1] |
| 307 | 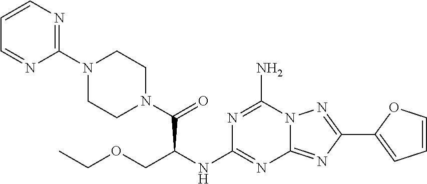 [SMILES: CCOC[C@H](Nc1nc(N)n2nc(-c3ccco3)nc2n1)C(=O)N1CCN(c2ncccn2)CC1] |
| 308 | 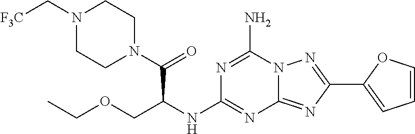 [SMILES: CCOC[C@H](Nc1nc(N)n2nc(-c3ccco3)nc2n1)C(=O)N1CCN(CC(F)(F)F)CC1] |
| 309 | 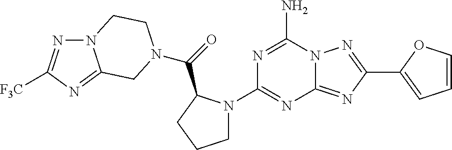 [SMILES: Nc1nc(N2CCC[C@H]2C(=O)N2CCn3nc(C(F)(F)F)nc3C2)nc2nc(-c3ccco3)nn12] |
| 310 | 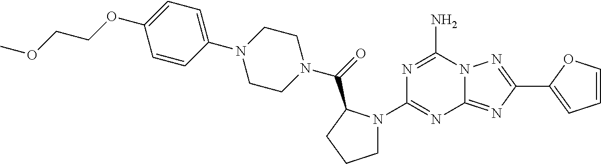 [SMILES: COCCOc1ccc(N2CCN(C(=O)[C@@H]3CCCN3c3nc(N)n4nc(-c5ccco5)nc4n3)CC2)cc1] |
| 311 | 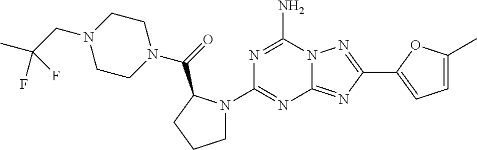 [SMILES: Cc1ccc(-c2nc3nc(N4CCC[C@H]4C(=O)N4CCN(CC(C)(F)F)CC4)nc(N)n3n2)o1] |
| 312 | 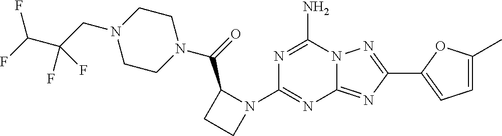 [SMILES: Cc1ccc(-c2nc3nc(N4CC[C@H]4C(=O)N4CCN(CC(F)(F)C(F)F)CC4)nc(N)n3n2)o1] |
| 313 | 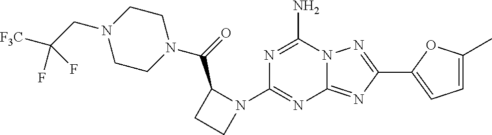 [SMILES: Cc1ccc(-c2nc3nc(N4CC[C@H]4C(=O)N4CCN(CC(F)(F)C(F)(F)F)CC4)nc(N)n3n2)o1] |
| 314 | 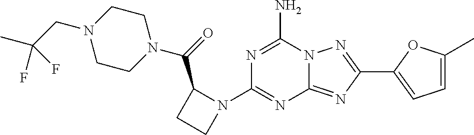 [SMILES: Cc1ccc(-c2nc3nc(N4CC[C@H]4C(=O)N4CCN(CC(C)(F)F)CC4)nc(N)n3n2)o1] |
| 315 | 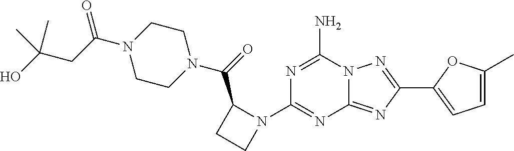 [SMILES: Cc1ccc(-c2nc3nc(N4CC[C@H]4C(=O)N4CCN(C(=O)CC(C)(C)O)CC4)nc(N)n3n2)o1] |
| 316 | 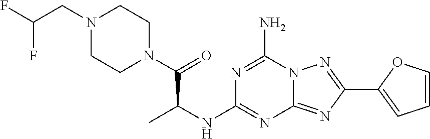 [SMILES: C[C@H](Nc1nc(N)n2nc(-c3ccco3)nc2n1)C(=O)N1CCN(CC(F)F)CC1] |
| 317 | 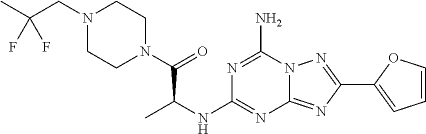 [SMILES: C[C@H](Nc1nc(N)n2nc(-c3ccco3)nc2n1)C(=O)N1CCN(CC(C)(F)F)CC1] |
| 318 | 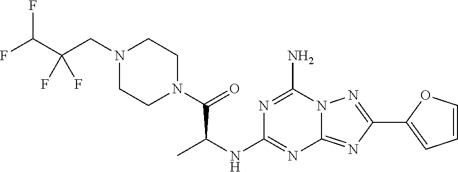 [SMILES: C[C@H](Nc1nc(N)n2nc(-c3ccco3)nc2n1)C(=O)N1CCN(CC(F)(F)C(F)F)CC1] |
| 319 | 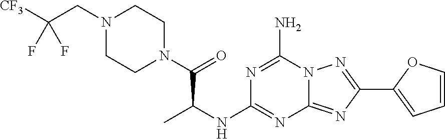 [SMILES: C[C@H](Nc1nc(N)n2nc(-c3ccco3)nc2n1)C(=O)N1CCN(CC(F)(F)C(F)(F)F)CC1] |
| 320 | 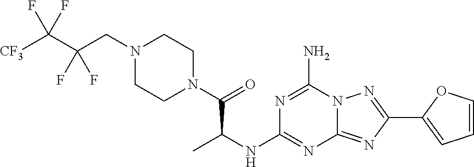 [SMILES: C[C@H](Nc1nc(N)n2nc(-c3ccco3)nc2n1)C(=O)N1CCN(CC(F)(F)C(F)(F)C(F)(F)F)CC1] |
| 321 | 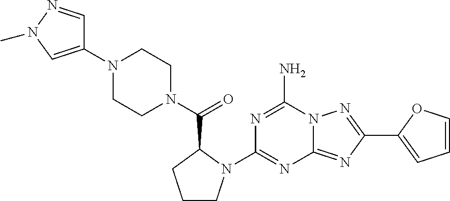 [SMILES: Cn1cc(N2CCN(C(=O)[C@@H]3CCCN3c3nc(N)n4nc(-c5ccco5)nc4n3)CC2)cn1] |
| 322 | 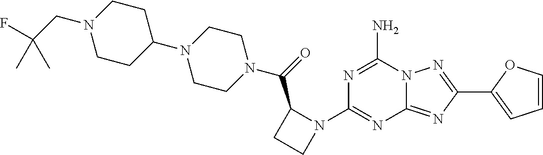 [SMILES: CC(C)(F)CN1CCC(N2CCN(C(=O)[C@@H]3CCN3c3nc(N)n4nc(-c5ccco5)nc4n3)CC2)CC1] |
| 323 | 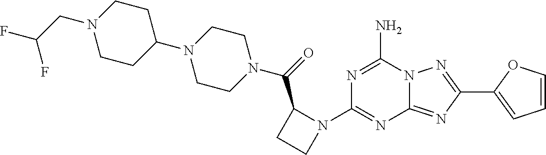 [SMILES: Nc1nc(N2CC[C@H]2C(=O)N2CCN(C3CCN(CC(F)F)CC3)CC2)nc2nc(-c3ccco3)nn12] |
| 324 | 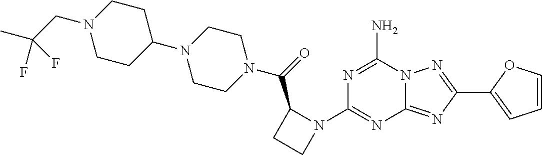 [SMILES: CC(F)(F)CN1CCC(N2CCN(C(=O)[C@@H]3CCN3c3nc(N)n4nc(-c5ccco5)nc4n3)CC2)CC1] |
| 325 | 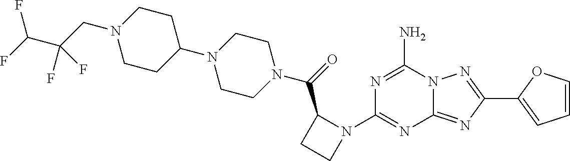 [SMILES: Nc1nc(N2CC[C@H]2C(=O)N2CCN(C3CCN(CC(F)(F)C(F)F)CC3)CC2)nc2nc(-c3ccco3)nn12] |
| 326 | 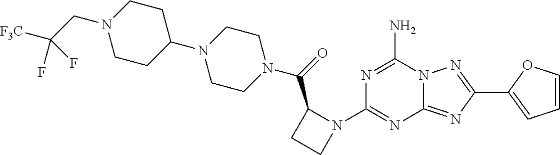 [SMILES: Nc1nc(N2CC[C@H]2C(=O)N2CCN(C3CCN(CC(F)(F)C(F)(F)F)CC3)CC2)nc2nc(-c3ccco3)nn12] |
| 327 | 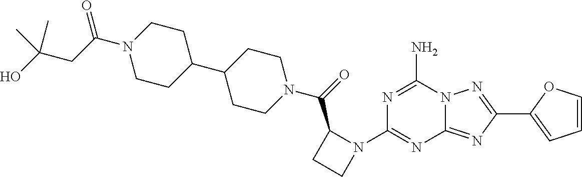 [SMILES: CC(C)(O)CC(=O)N1CCC(C2CCN(C(=O)[C@@H]3CCN3c3nc(N)n4nc(-c5ccco5)nc4n3)CC2)CC1] |
| 328 | 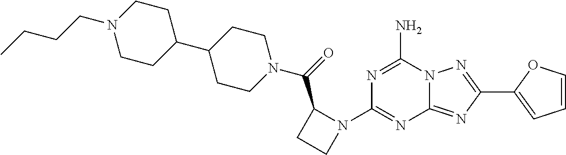 [SMILES: CCCCN1CCC(C2CCN(C(=O)[C@@H]3CCN3c3nc(N)n4nc(-c5ccco5)nc4n3)CC2)CC1] |
| 329 | 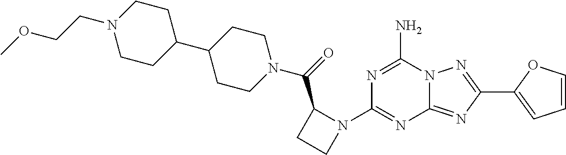 [SMILES: COCCN1CCC(C2CCN(C(=O)[C@@H]3CCN3c3nc(N)n4nc(-c5ccco5)nc4n3)CC2)CC1] |
| 330 | 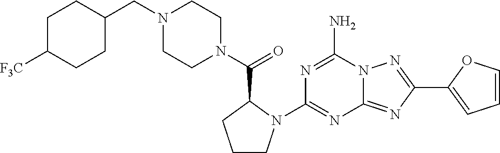 [SMILES: Nc1nc(N2CCC[C@H]2C(=O)N2CCN(CC3CCC(C(F)(F)F)CC3)CC2)nc2nc(-c3ccco3)nn12] |
| 331 | 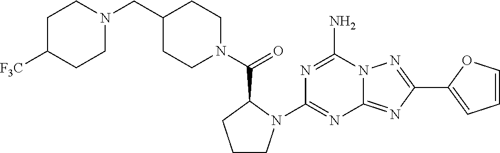 [SMILES: Nc1nc(N2CCC[C@H]2C(=O)N2CCC(CN3CCC(C(F)(F)F)CC3)CC2)nc2nc(-c3ccco3)nn12] |
| 332 | 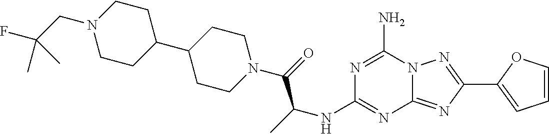 [SMILES: C[C@H](Nc1nc(N)n2nc(-c3ccco3)nc2n1)C(=O)N1CCC(C2CCN(CC(C)(C)F)CC2)CC1] |
| 333 | 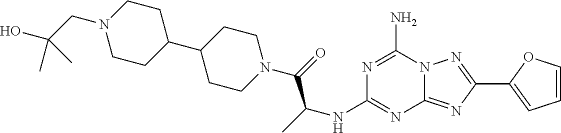 [SMILES: C[C@H](Nc1nc(N)n2nc(-c3ccco3)nc2n1)C(=O)N1CCC(C2CCN(CC(C)(C)O)CC2)CC1] |
| 334 | 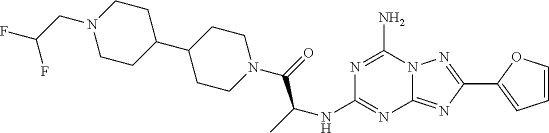 [SMILES: C[C@H](Nc1nc(N)n2nc(-c3ccco3)nc2n1)C(=O)N1CCC(C2CCN(CC(F)F)CC2)CC1] |
| 335 | 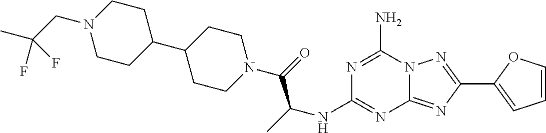 [SMILES: C[C@H](Nc1nc(N)n2nc(-c3ccco3)nc2n1)C(=O)N1CCC(C2CCN(CC(C)(F)F)CC2)CC1] |
| 336 | 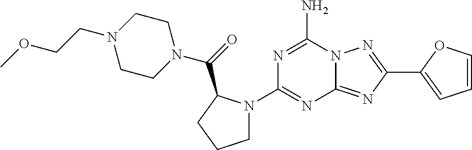 [SMILES: COCCN1CCN(C(=O)[C@@H]2CCCN2c2nc(N)n3nc(-c4ccco4)nc3n2)CC1] |
| 337 | 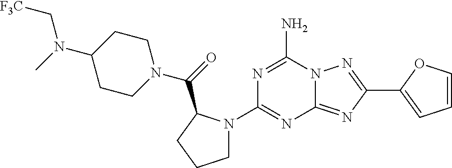 [SMILES: CN(CC(F)(F)F)C1CCN(C(=O)[C@@H]2CCCN2c2nc(N)n3nc(-c4ccco4)nc3n2)CC1] |
| 338 | 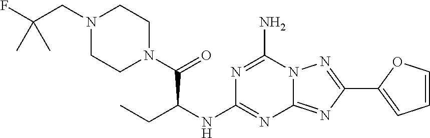 [SMILES: CC[C@H](Nc1nc(N)n2nc(-c3ccco3)nc2n1)C(=O)N1CCN(CC(C)(C)F)CC1] |
| 339 | 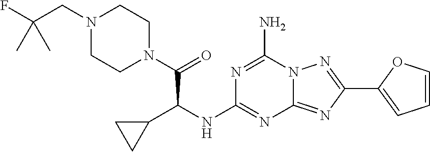 [SMILES: CC(C)(F)CN1CCN(C(=O)[C@@H](Nc2nc(N)n3nc(-c4ccco4)nc3n2)C2CC2)CC1] |
| 340 | 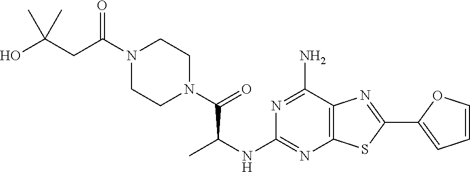 [SMILES: C[C@H](Nc1nc(N)c2nc(-c3ccco3)sc2n1)C(=O)N1CCN(C(=O)CC(C)(C)O)CC1] |
| 341 | 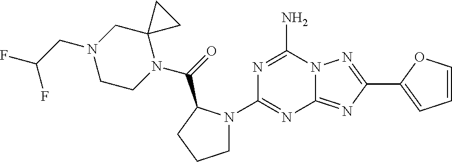 [SMILES: Nc1nc(N2CCC[C@H]2C(=O)N2CCN(CC(F)F)CC23CC3)nc2nc(-c3ccco3)nn12] |
| 342 | 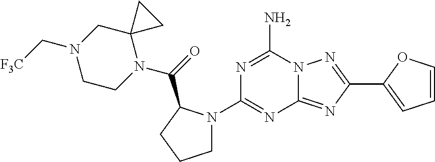 [SMILES: Nc1nc(N2CCC[C@H]2C(=O)N2CCN(CC(F)(F)F)CC23CC3)nc2nc(-c3ccco3)nn12] |
| 343 | 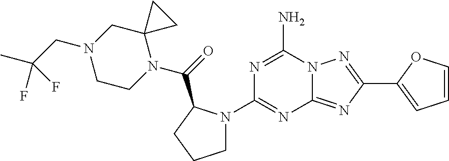 [SMILES: CC(F)(F)CN1CCN(C(=O)[C@@H]2CCCN2c2nc(N)n3nc(-c4ccco4)nc3n2)C2(CC2)C1] |
| 344 | 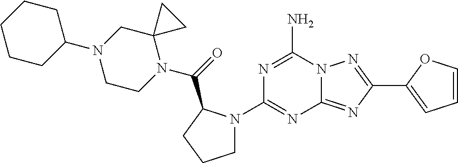 [SMILES: Nc1nc(N2CCC[C@H]2C(=O)N2CCN(C3CCCCC3)CC23CC3)nc2nc(-c3ccco3)nn12] |
| 345 | 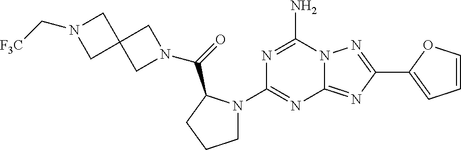 [SMILES: Nc1nc(N2CCC[C@H]2C(=O)N2CC3(CN(CC(F)(F)F)C3)C2)nc2nc(-c3ccco3)nn12] |
| 346 | 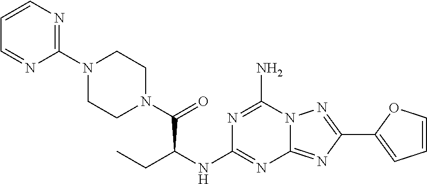 [SMILES: CC[C@H](Nc1nc(N)n2nc(-c3ccco3)nc2n1)C(=O)N1CCN(c2ncccn2)CC1] |
| 347 | 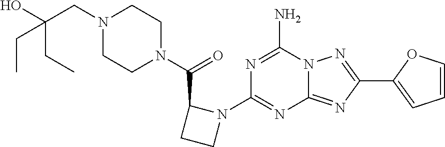 [SMILES: CCC(O)(CC)CN1CCN(C(=O)[C@@H]2CCN2c2nc(N)n3nc(-c4ccco4)nc3n2)CC1] |
| 348 | 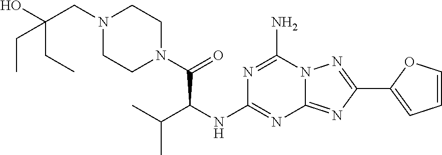 [SMILES: CCC(O)(CC)CN1CCN(C(=O)[C@@H](Nc2nc(N)n3nc(-c4ccco4)nc3n2)C(C)C)CC1] |
| 349 | 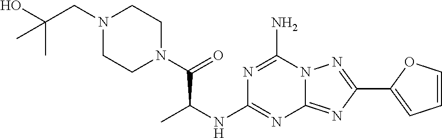 [SMILES: C[C@H](Nc1nc(N)n2nc(-c3ccco3)nc2n1)C(=O)N1CCN(CC(C)(C)O)CC1] |
| 350 | 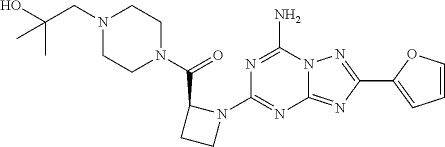 [SMILES: CC(C)(O)CN1CCN(C(=O)[C@@H]2CCN2c2nc(N)n3nc(-c4ccco4)nc3n2)CC1] |
| 351 | 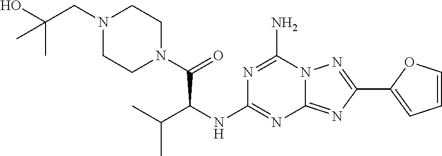 [SMILES: CC(C)[C@H](Nc1nc(N)n2nc(-c3ccco3)nc2n1)C(=O)N1CCN(CC(C)(C)O)CC1] |
| 352 | 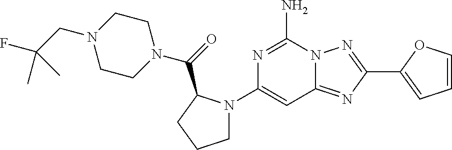 [SMILES: CC(C)(F)CN1CCN(C(=O)[C@@H]2CCCN2c2cc3nc(-c4ccco4)nn3c(N)n2)CC1] |
| 353 | 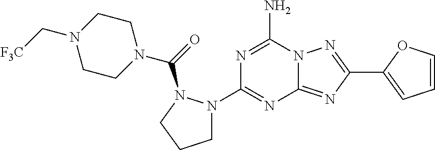 |
| 354 | 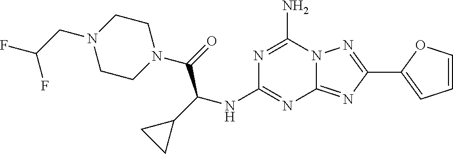 [SMILES: Nc1nc(N[C@H](C(=O)N2CCN(CC(F)F)CC2)C2CC2)nc2nc(-c3ccco3)nn12] |
| 355 | 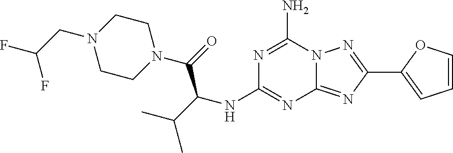 [SMILES: CC(C)[C@H](Nc1nc(N)n2nc(-c3ccco3)nc2n1)C(=O)N1CCN(CC(F)F)CC1] |
| 356 | 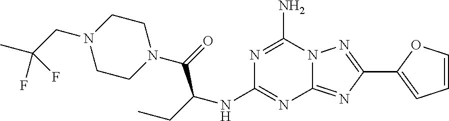 [SMILES: CC[C@H](Nc1nc(N)n2nc(-c3ccco3)nc2n1)C(=O)N1CCN(CC(C)(F)F)CC1] |
| 357 | 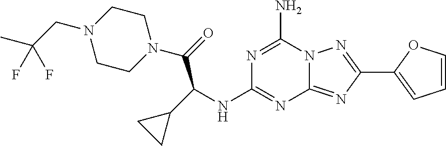 [SMILES: CC(F)(F)CN1CCN(C(=O)[C@@H](Nc2nc(N)n3nc(-c4ccco4)nc3n2)C2CC2)CC1] |
| 358 | 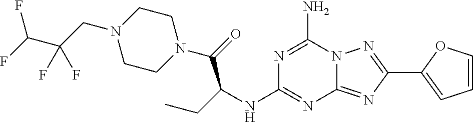 [SMILES: CC[C@H](Nc1nc(N)n2nc(-c3ccco3)nc2n1)C(=O)N1CCN(CC(F)(F)C(F)F)CC1] |
| 359 | 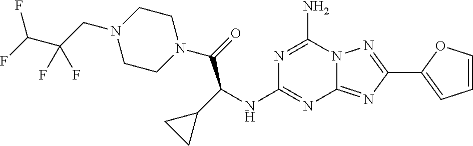 [SMILES: Nc1nc(N[C@H](C(=O)N2CCN(CC(F)(F)C(F)F)CC2)C2CC2)nc2nc(-c3ccco3)nn12] |
| 360 | 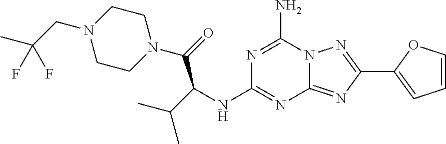 [SMILES: CC(C)[C@H](Nc1nc(N)n2nc(-c3ccco3)nc2n1)C(=O)N1CCN(CC(C)(F)F)CC1] |
| 361 | 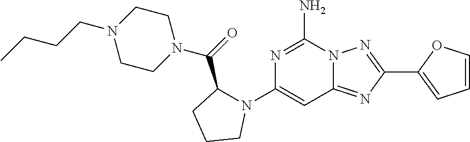 [SMILES: CCCCN1CCN(C(=O)[C@@H]2CCCN2c2cc3nc(-c4ccco4)nn3c(N)n2)CC1] |
| 362 | 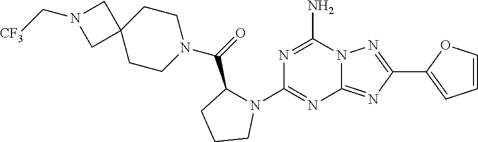 [SMILES: Nc1nc(N2CCC[C@H]2C(=O)N2CCC3(CC2)CN(CC(F)(F)F)C3)nc2nc(-c3ccco3)nn12] |
| 363 | 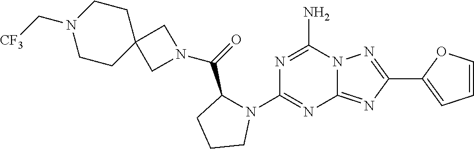 [SMILES: Nc1nc(N2CCC[C@H]2C(=O)N2CC3(CCN(CC(F)(F)F)CC3)C2)nc2nc(-c3ccco3)nn12] |
| 364 | 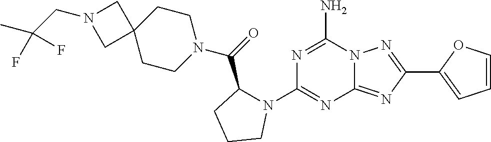 [SMILES: CC(F)(F)CN1CC2(CCN(C(=O)[C@@H]3CCCN3c3nc(N)n4nc(-c5ccco5)nc4n3)CC2)C1] |
| 365 | 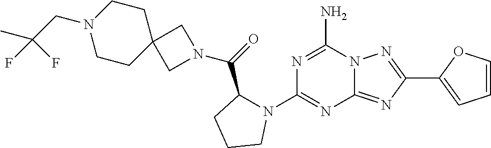 [SMILES: CC(F)(F)CN1CCC2(CC1)CN(C(=O)[C@@H]1CCCN1c1nc(N)n3nc(-c4ccco4)nc3n1)C2] |
| 366 | 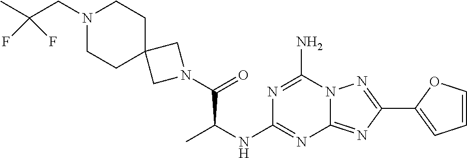 [SMILES: C[C@H](Nc1nc(N)n2nc(-c3ccco3)nc2n1)C(=O)N1CC2(CCN(CC(C)(F)F)CC2)C1] |
| 367 | 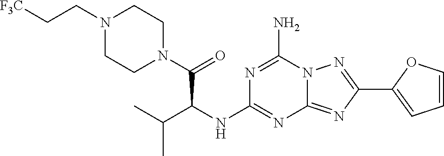 [SMILES: CC(C)[C@H](Nc1nc(N)n2nc(-c3ccco3)nc2n1)C(=O)N1CCN(CCC(F)(F)F)CC1] |
| 368 | 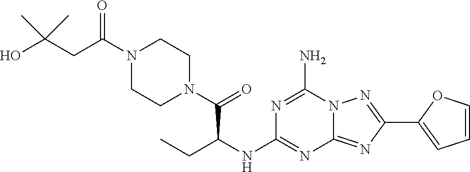 [SMILES: CC[C@H](Nc1nc(N)n2nc(-c3ccco3)nc2n1)C(=O)N1CCN(C(=O)CC(C)(C)O)CC1] |
| 369 | 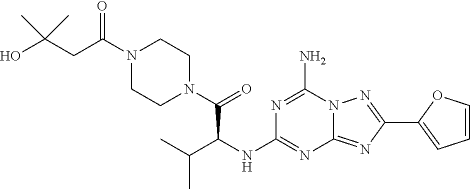 [SMILES: CC(C)[C@H](Nc1nc(N)n2nc(-c3ccco3)nc2n1)C(=O)N1CCN(C(=O)CC(C)(C)O)CC1] |
| 370 | 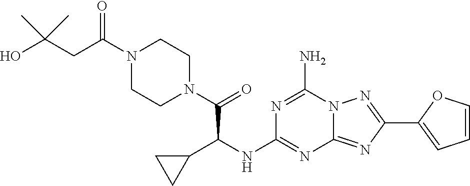 [SMILES: CC(C)(O)CC(=O)N1CCN(C(=O)[C@@H](Nc2nc(N)n3nc(-c4ccco4)nc3n2)C2CC2)CC1] |
| 371 | 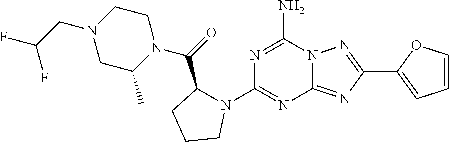 [SMILES: C[C@@H]1CN(CC(F)F)CCN1C(=O)[C@@H]1CCCN1c1nc(N)n2nc(-c3ccco3)nc2n1] |
| 372 | 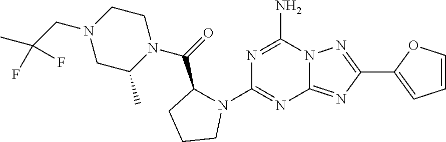 [SMILES: C[C@@H]1CN(CC(C)(F)F)CCN1C(=O)[C@@H]1CCCN1c1nc(N)n2nc(-c3ccco3)nc2n1] |
| 373 | 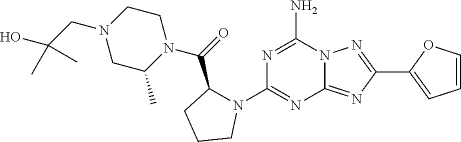 [SMILES: C[C@@H]1CN(CC(C)(C)O)CCN1C(=O)[C@@H]1CCCN1c1nc(N)n2nc(-c3ccco3)nc2n1] |
| 374 | 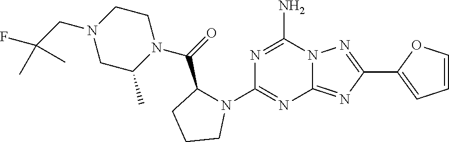 [SMILES: C[C@@H]1CN(CC(C)(C)F)CCN1C(=O)[C@@H]1CCCN1c1nc(N)n2nc(-c3ccco3)nc2n1] |
| 375 | 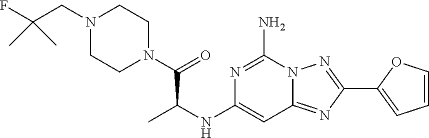 [SMILES: C[C@H](Nc1cc2nc(-c3ccco3)nn2c(N)n1)C(=O)N1CCN(CC(C)(C)F)CC1] |
| 376 | 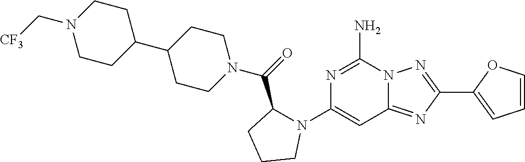 [SMILES: Nc1nc(N2CCC[C@H]2C(=O)N2CCC(C3CCN(CC(F)(F)F)CC3)CC2)cc2nc(-c3ccco3)nn12] |
| 377 | 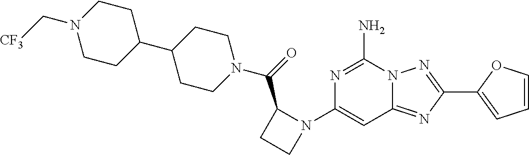 [SMILES: Nc1nc(N2CC[C@H]2C(=O)N2CCC(C3CCN(CC(F)(F)F)CC3)CC2)cc2nc(-c3ccco3)nn12] |
| 378 | 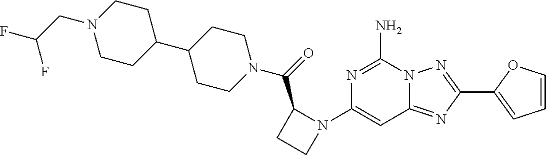 [SMILES: Nc1nc(N2CC[C@H]2C(=O)N2CCC(C3CCN(CC(F)F)CC3)CC2)cc2nc(-c3ccco3)nn12] |
| 379 | 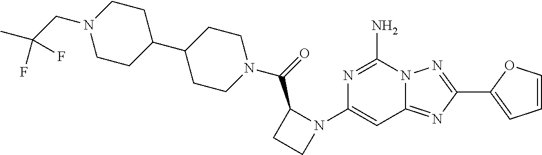 [SMILES: CC(F)(F)CN1CCC(C2CCN(C(=O)[C@@H]3CCN3c3cc4nc(-c5ccco5)nn4c(N)n3)CC2)CC1] |
| 380 | 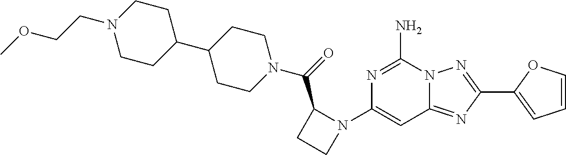 [SMILES: COCCN1CCC(C2CCN(C(=O)[C@@H]3CCN3c3cc4nc(-c5ccco5)nn4c(N)n3)CC2)CC1] |
| 381 | 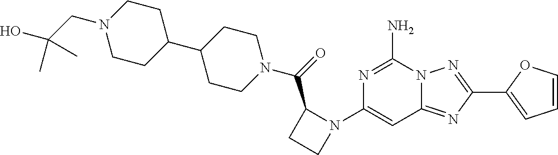 [SMILES: CC(C)(O)CN1CCC(C2CCN(C(=O)[C@@H]3CCN3c3cc4nc(-c5ccco5)nn4c(N)n3)CC2)CC1] |
| 382 | 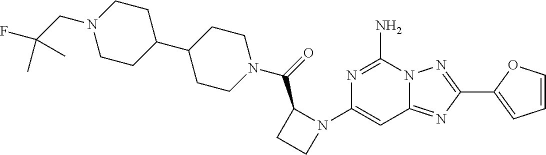 [SMILES: CC(C)(F)CN1CCC(C2CCN(C(=O)[C@@H]3CCN3c3cc4nc(-c5ccco5)nn4c(N)n3)CC2)CC1] |
| 383 | 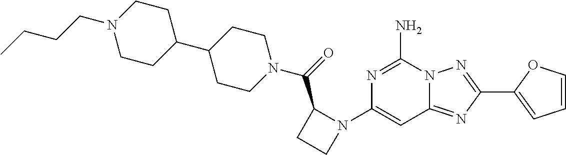 [SMILES: CCCCN1CCC(C2CCN(C(=O)[C@@H]3CCN3c3cc4nc(-c5ccco5)nn4c(N)n3)CC2)CC1] |
| 384 | 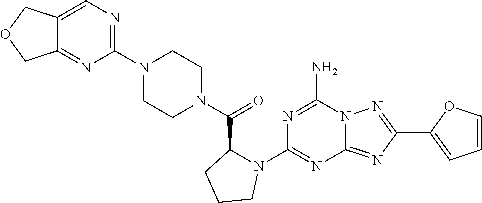 [SMILES: Nc1nc(N2CCC[C@H]2C(=O)N2CCN(c3ncc4c(n3)COC4)CC2)nc2nc(-c3ccco3)nn12] |
| 385 | 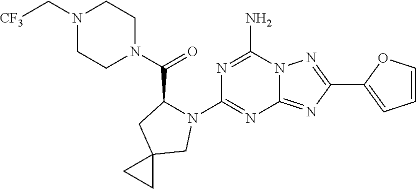 [SMILES: Nc1nc(N2CC3(CC3)C[C@H]2C(=O)N2CCN(CC(F)(F)F)CC2)nc2nc(-c3ccco3)nn12] |
| 386 | 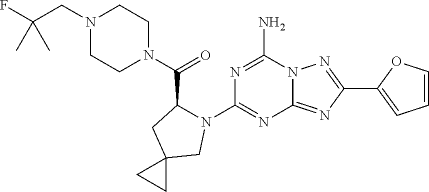 [SMILES: CC(C)(F)CN1CCN(C(=O)[C@@H]2CC3(CC3)CN2c2nc(N)n3nc(-c4ccco4)nc3n2)CC1] |
| 387 | 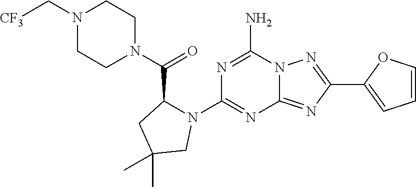 [SMILES: CC1(C)C[C@@H](C(=O)N2CCN(CC(F)(F)F)CC2)N(c2nc(N)n3nc(-c4ccco4)nc3n2)C1] |
| 388 | 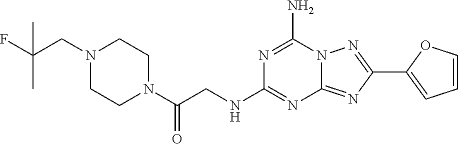 [SMILES: CC(C)(F)CN1CCN(C(=O)CNc2nc(N)n3nc(-c4ccco4)nc3n2)CC1] |
| 389 | 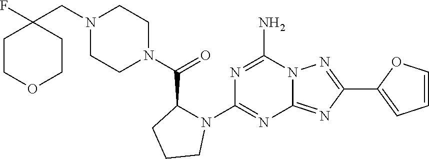 [SMILES: Nc1nc(N2CCC[C@H]2C(=O)N2CCN(CC3(F)CCOCC3)CC2)nc2nc(-c3ccco3)nn12] |
| 390 | 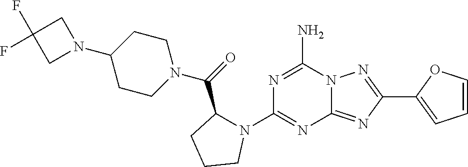 [SMILES: Nc1nc(N2CCC[C@H]2C(=O)N2CCC(N3CC(F)(F)C3)CC2)nc2nc(-c3ccco3)nn12] |
| 391 | 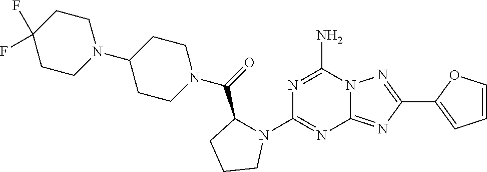 [SMILES: Nc1nc(N2CCC[C@H]2C(=O)N2CCC(N3CCC(F)(F)CC3)CC2)nc2nc(-c3ccco3)nn12] |
| 392 | 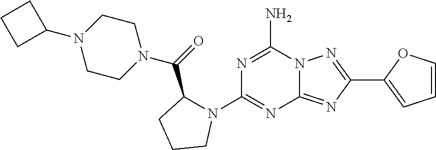 [SMILES: Nc1nc(N2CCC[C@H]2C(=O)N2CCN(C3CCC3)CC2)nc2nc(-c3ccco3)nn12] |
| 393 | 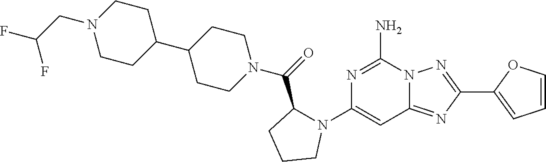 [SMILES: Nc1nc(N2CCC[C@H]2C(=O)N2CCC(C3CCN(CC(F)F)CC3)CC2)cc2nc(-c3ccco3)nn12] |
| 394 | 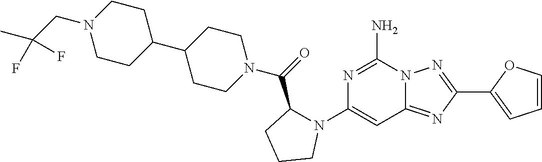 [SMILES: CC(F)(F)CN1CCC(C2CCN(C(=O)[C@@H]3CCCN3c3cc4nc(-c5ccco5)nn4c(N)n3)CC2)CC1] |
| 395 | 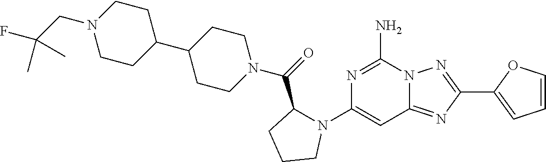 [SMILES: CC(C)(F)CN1CCC(C2CCN(C(=O)[C@@H]3CCCN3c3cc4nc(-c5ccco5)nn4c(N)n3)CC2)CC1] |
| 396 | 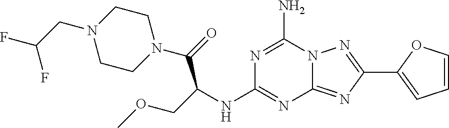 [SMILES: COC[C@H](Nc1nc(N)n2nc(-c3ccco3)nc2n1)C(=O)N1CCN(CC(F)F)CC1] |
| 397 | 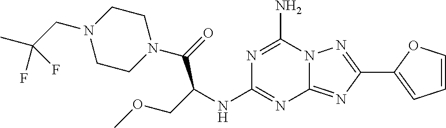 [SMILES: COC[C@H](Nc1nc(N)n2nc(-c3ccco3)nc2n1)C(=O)N1CCN(CC(C)(F)F)CC1] |
| 398 | 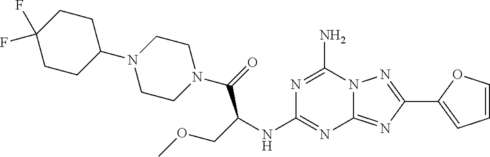 [SMILES: COC[C@H](Nc1nc(N)n2nc(-c3ccco3)nc2n1)C(=O)N1CCN(C2CCC(F)(F)CC2)CC1] |
| 399 | 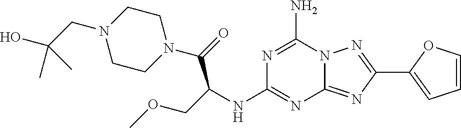 [SMILES: COC[C@H](Nc1nc(N)n2nc(-c3ccco3)nc2n1)C(=O)N1CCN(CC(C)(C)O)CC1] |
| 400 | 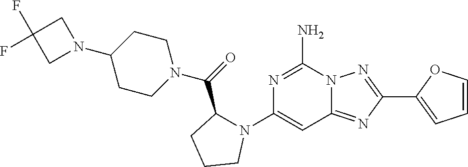 [SMILES: Nc1nc(N2CCC[C@H]2C(=O)N2CCC(N3CC(F)(F)C3)CC2)cc2nc(-c3ccco3)nn12] |
| 401 | 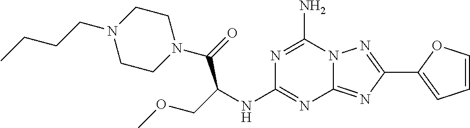 [SMILES: CCCCN1CCN(C(=O)[C@H](COC)Nc2nc(N)n3nc(-c4ccco4)nc3n2)CC1] |
| 402 | 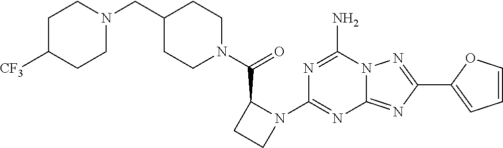 [SMILES: Nc1nc(N2CC[C@H]2C(=O)N2CCC(CN3CCC(C(F)(F)F)CC3)CC2)nc2nc(-c3ccco3)nn12] |
| 403 | 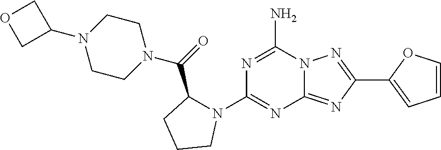 [SMILES: Nc1nc(N2CCC[C@H]2C(=O)N2CCN(C3COC3)CC2)nc2nc(-c3ccco3)nn12] |
| 404 | 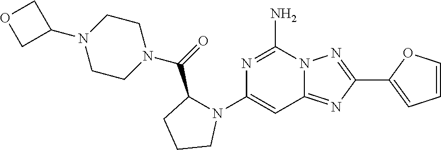 [SMILES: Nc1nc(N2CCC[C@H]2C(=O)N2CCN(C3COC3)CC2)cc2nc(-c3ccco3)nn12] |
| 405 | 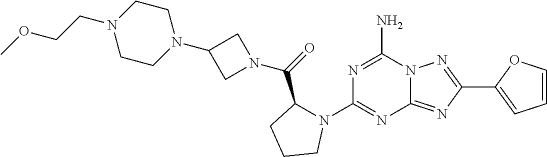 [SMILES: COCCN1CCN(C2CN(C(=O)[C@@H]3CCCN3c3nc(N)n4nc(-c5ccco5)nc4n3)C2)CC1] |
| 406 | 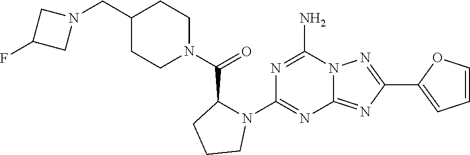 [SMILES: Nc1nc(N2CCC[C@H]2C(=O)N2CCC(CN3CC(F)C3)CC2)nc2nc(-c3ccco3)nn12] |

(6) The compound represented by formula 1, stereoisomers thereof or pharmaceutically acceptable salts thereof according to any one of above (1), (2), (3), (4), and (5), in which the compound represented by above formula 1, stereoisomers thereof or pharmaceutically acceptable salts thereof may include example compounds 25, 26, 48, 90, 111, 223, 224, 294, 303, 353 or 371.

In one embodiment, the compound represented by above formula 1, stereoisomers thereof or pharmaceutically acceptable salts thereof may include example compounds 4, 6, 10, 11, 13, 14, 1′7, 18, 32, 77, 123, 149, 150, 163, 164, 165, 166, 167 or 169.

The present invention may provide a compound as an A2a receptor antagonist, stereoisomers thereof or pharmaceutically acceptable salts thereof may be at least one compound selected from the compounds shown in the table 1 above.

In the present invention, “pharmaceutically acceptable salts” may mean the salts conventionally used in a pharmaceutical industry, for example, inorganic ion salts prepared from calcium, potassium, sodium, magnesium and the like; inorganic acid salts prepared from hydrochloric acid, nitric acid, phosphoric acid, bromic acid, iodic acid, perchloric acid, tartaric acid, sulfuric acid and the like; organic acid salts prepared from acetic acid, trifluoroacetic acid, citric acid, maleic acid, succinic acid, oxalic acid, benzoic acid, fumaric acid, mandelic acid, propionic acid, lactic acid, glycolic acid, gluconic acid, galacturonic acid, glutamic acid, glutaric acid, glucuronic acid, aspartic acid, ascorbic acid, carbonic acid, vanillic acid, hydroiodic acid, etc.; sulfonic acid salts prepared from methanesulfonic acid, ethanesulfonic acid, benzenesulfonic acid, p-toluenesulfonic acid, naphthalenesulfonic acid and the like; amino acid salts prepared from glycine, arginine, lysine, etc.; amine salts prepared from trimethylamine, triethylamine, ammonia, pyridine, picoline, etc.; and the like, but types of salts meant in the present invention are not limited to those listed salts.

In the present invention, “stereoisomer” may include a diastereomer and an optical isomer (enantiomer), in which the optical isomer may include not only an enantiomer, but also a mixture of the enantiomer and even a racemate. Such isomer may be separated by being split according to the related art, for example, column chromatography, HPLC or the like. Alternatively, a stereoisomer of each of the compound represented by formula 1 may be stereospecifically synthesized by using a known array of optically pure starting materials and/or reagents.

In the present invention, the compound as an A2a receptor antagonist may be the same as the compound list in this specification, but also include a pharmaceutically acceptable isotopic-labeled compound in which at least one may be replaced with an atom having the same atomic number, but having an atomic mass or mass number different from the atomic mass or mass number prevailing in nature. Examples of isotopes which may be included in the compound of the present invention may include: ²H, ³H, isotopes of hydrogen; ¹¹C, ¹³C, ¹⁴C, isotopes of carbon; ³⁶Cl, an isotope of chlorine; ¹⁸F, an isotope of fluorine; ¹²³I, ¹²⁵I, isotopes of iodine; ¹³N, ¹⁵N, isotopes of nitrogen; ¹⁵O, ¹⁷O, ¹⁸O, isotopes of oxygen; ³²P, an isotope of phosphorus; ³⁵S, an isotope of sulfur; and the like. A certain isotopic-labeled compound of the present invention, for example, a compound with radioactive isotopes incorporated, may be useful in studying drugs and/or a distribution of substrate tissues (e.g., assays). A radioactive isotope tritium, that is, ³H, and carbon-14, that is, ¹⁴C may be useful in view of ease of incorporation and means of immediate detection. Substitution with heavier isotopes, for example, substitution of hydrogen (¹H) with deuterium (²H), may exhibit an excellent therapeutic effect on diseases by enhancing metabolic stability, such as increasing a half-life in vivo or reducing a dosage. Substitution with positron-emitting isotopes, for example, ¹¹C, ¹⁵F, ¹⁸F, ¹⁵O, ¹³N, etc., may be useful in studying positron emission tomography (PET) to examine a substrate receptor occupancy. An isotopic-labeled compound of the present invention may be generally prepared by conventional techniques known to those skilled in the art, by processes similar to those described in the reaction formulas and/or examples and preparation examples described in this specification, using an appropriate isotopic-labeled reagent instead of the non-labeled reagent as used in this specification. Compounds represented by formula 1 and compounds exemplified in this specification, may include isotopic-labeled compounds of these compounds, such as, but not limited to, compounds including deuterated and tritiated isotopes and all other isotopes discussed above.

Method for Preparing Compound Represented by Formula 1

The present invention may provide a method for preparing a compound represented by formula 1, stereoisomers thereof or pharmaceutically acceptable salts thereof.

The compound represented by formula 1, stereoisomers thereof or pharmaceutically acceptable salts thereof may be prepared according to any one method of reaction formulas 1 to 10, which may be modified to a level apparent to those skilled in the art.

In reaction formulas 1 to 10 below, R₁ to R₇, Z₁ to Z₃, W₁ to W₄, Q, La, Ra to Rh, a, b, m, n, q, r, t, s, u, y, L₁ and L₂ may be each substantially the same as defined in formula 1, unless particularly defined. The “PG” may mean a protecting group and may include tert-butyloxycarbonyl (Boc), benzyloxycarbonyl (Cbz) or the like.

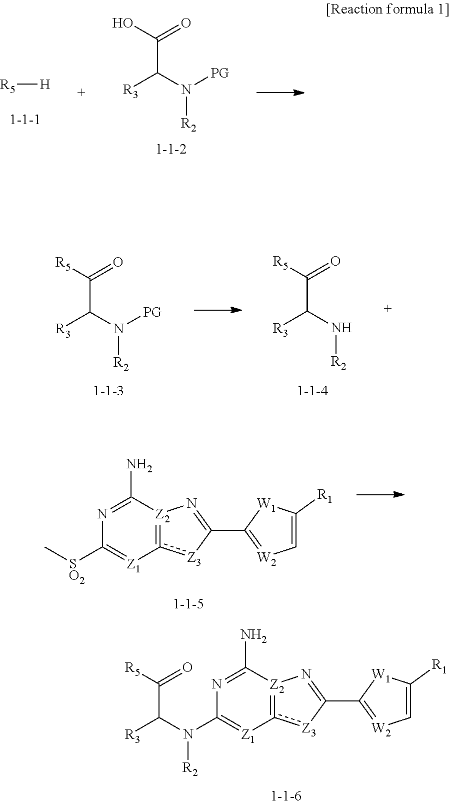

According to above reaction formula 1, a compound of formula 1-1-1 (R₅—H) may be reacted with formula 1-1-2 to prepare a compound of formula 1-1-3, after which a protecting group (PG) may be removed therefrom to prepare a compound of formula 1-1-4, which may be then subjected to a substitution reaction with a compound of formula 1-1-5, thereby preparing a compound of formula 1-1-6.

In the present invention, examples of the compounds prepared according to the same method as shown in above reaction formula 1 may include example compounds 1 to 3, 7 to 16, 19, 20, 26 to 29, 37, 40, 41, 48, 55, 57 to 60, 65, 67, 78 to 81, 84, 87, 90 to 98, 107 to 110, 112 to 114, 118 to 128, 134 to 151, 162 to 178, 187 to 195, 199 to 203, 207 to 222, 231 to 236, 238 to 243, 246 to 251, 264, 281, 300 to 310, 321, 336, 338, 339, 346, 349 to 352, 361, 367, 375, 385 to 399, 401 or the like.

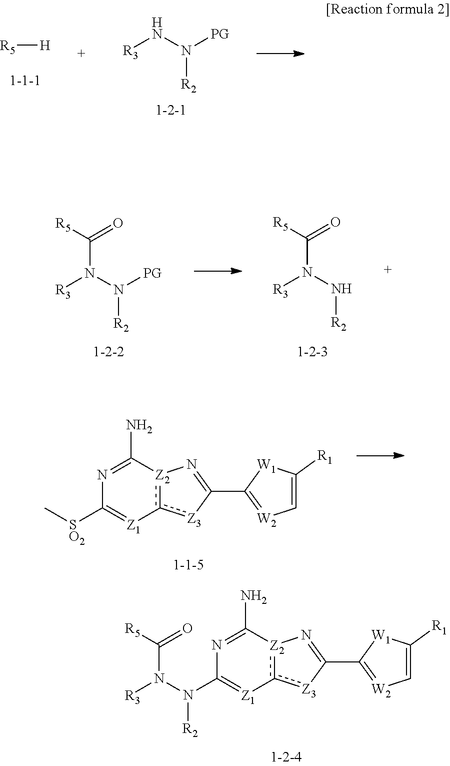

Above reaction formula 2 may show a synthesis method of a pyrazolidin-1-carboxamide compound, in which a compound of formula 1-1-1 and formula 1-2-1 may be subjected to a reaction to prepare a compound of formula 1-2-2, after which a protecting group may be removed therefrom, so as to prepare a compound of formula 1-2-3. After that, a compound of formula 1-2-4 may be prepared through a substitution reaction with a compound of formula 1-1-5.

In the present invention, a compound prepared by the same method as shown in above reaction formula 2 may include example compound 353, etc.

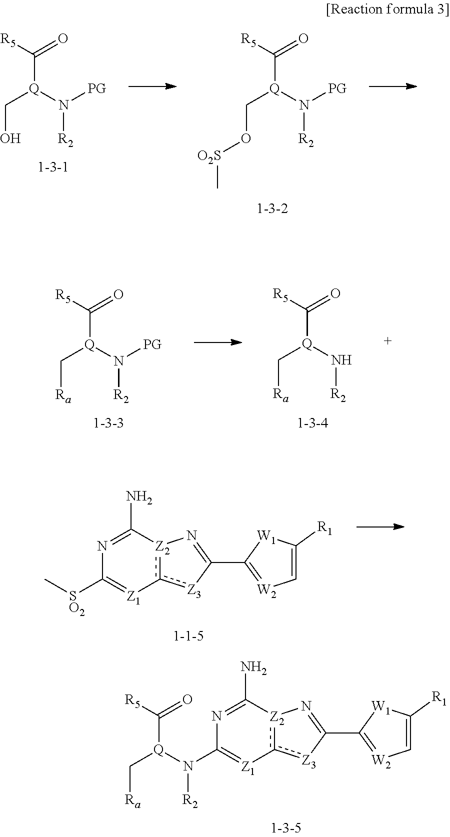

(In above reaction formula 3, Ra may represent

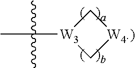

According to above reaction formula 3, a compound of formula 1-3-1 may be subjected to a methane sulfonylation reaction to obtain a compound of formula 1-3-2, which may be then subjected to a substitution reaction to prepare a compound of formula 1-3-3, after which a protecting group may be removed therefrom, so as to prepare a compound of formula 1-3-4. After that, a compound of formula 1-3-5 may be prepared through a substitution reaction with a compound of formula 1-1-5.

In the present invention, the compounds prepared by the same method as shown in above reaction formula 3 may include example compounds 272, 273, etc.

A compound of formula 1-1-1 represented by R₅—H in each of above reaction formulas 1 and 2 may be prepared according to the methods described in reaction formulas 4a to 4e below and reaction formulas 5a, 5b, and 6 to 8 below. In other words, a compound of formula 1-1 represented by R₅—H in each of above reaction formulas 1 and 2 may be a compound of formulas 1-4-7, 1-4-8, or 1-4-9 below, or may be a compound of formulas 1-5-5, 1-5-6, 1-6-3, 1-7-4, 1-7-5, or 1-8-3.

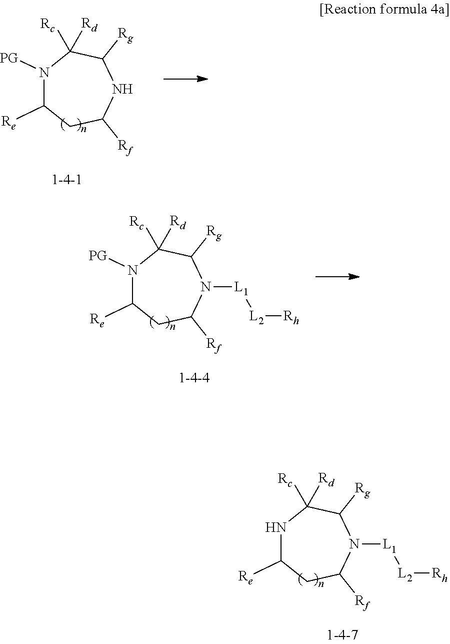

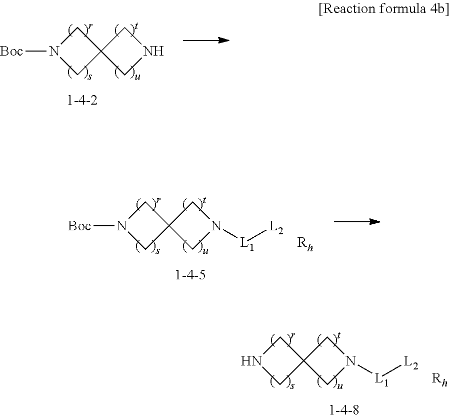

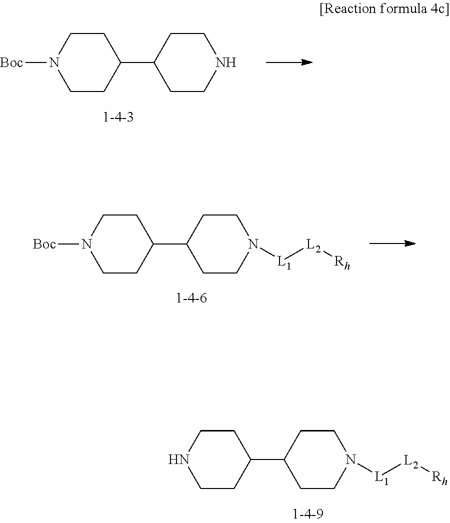

According to reaction formulas 4a to 4c, a substituent (-L₁-L₂-Rₕ) may be introduced into a compound of formula 1-4-1, 1-4-2 or 1-4-3 to prepare a compound of formula 1-4-4, 1-4-5 or 1-4-6, after which a protecting group (PG, Boc) may be removed therefrom, so as to prepare a compound of formula 1-4-7, 1-4-8 or 1-4-9 as a compound of formula 1-1-1 (R₅—H).

In the present invention, examples of compounds which may be synthesized according to a method as shown in above reaction formula 1 or 2 by using R₅—H prepared by the same method as shown above reaction formulas 4a to 4c may include example compounds 30 to 32, 42, 44, 49 to 52, 56, 73 to 77, 85, 86, 88, 129, 152 to 161, 179 to 184, 185, 196, 197, 204 to 206, 223 to 230, 237, 244, 252 to 263, 265 to 271, 274 to 280, 287 to 299, 311 to 320, 323 to 329, 333 to 335, 337, 340 to 345, 347, 348, 354 to 360, 362 to 366, 368 to 373, 376 to 381, 383, 384, 392 to 394, 396 to 398, 403, 404 or the like.

A substituent (-L₁-L₂-Rₕ) included in R₅ in each of above reaction formulas 4a to 4c may be introduced into a ring including N by using a coupling reaction with a compound having a halide compound such as acid chloride, oxalyl chloride, sulfonyl chloride, carbonyl chloride, etc., or a leaving group such as o-toluenesulfonyl fluoride, etc., a Buchwald-Hartwig reaction, a ring opening reaction through amide coupling and epoxide hydrolysis, a reductive amidation reaction, etc. For example, an introduction may be made into a ring including N by an alkylation or arylation reaction using a halide compound having a structure of X-L₁-L₂-Rₕ (in which X is halogen).

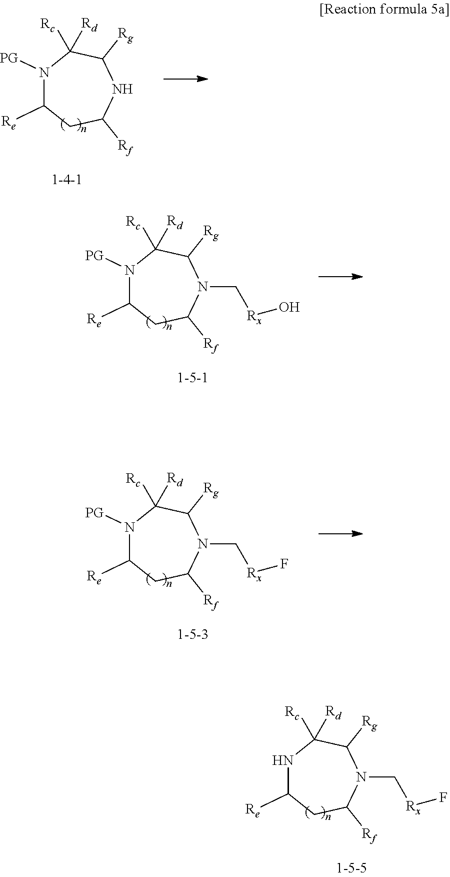

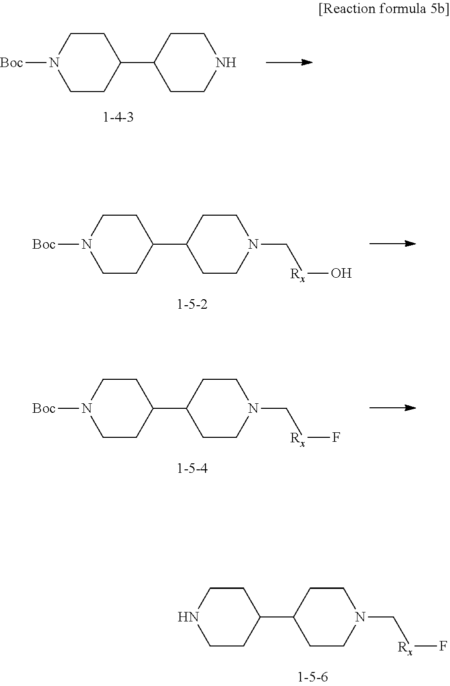

(Rx in above reaction formulas 5a and 5b may be each independently —C1-C7 alkylene-, and alkylene of Rx may mean a divalent substituent of straight or branched alkyl.)

According to above reaction formulas 5a and 5b, a substituent may be introduced into a compound of formula 1-4-1 or 1-4-3 to prepare a hydroxy compound of formula 1-5-1 or 1-5-2, which may be then subjected to a fluorination reaction to prepare a compound of formula 1-5-3 or 1-5-4, after which a protecting group (PG, Boc) may be removed therefrom, so as to prepare a compound of formula 1-5-5 or 1-5-6 as a compound of formula 1-1-1 (R₅—H).

In the present invention, examples of compounds with R₅ substituted as prepared by the same method as shown in above reaction formula 5a or 5b may include example compounds 43, 245, 322, 332, 374, 382, 395 or the like.

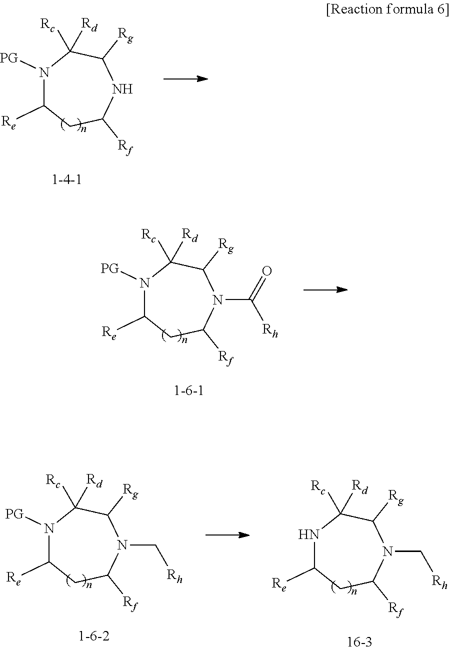

According to above reaction formula 6, a substituent may be introduced into a compound of formula 1-4-1 to prepare an amide compound of formula 1-6-1, which may be then subjected to a reduction reaction to prepare a compound of formula 1-6-2. A protecting group may be removed from the compound of formula 1-6-2 to prepare a compound of formula 1-6-3 as a compound of formula 1-1-1 (R₅—H).

In the present invention, examples of compounds which may be synthesized according to a method of above reaction formula 1 or 2 using R₅—H prepared by the same method as shown in above reaction formula 6 may include example compounds 282 to 286, 330, 389 or the like.

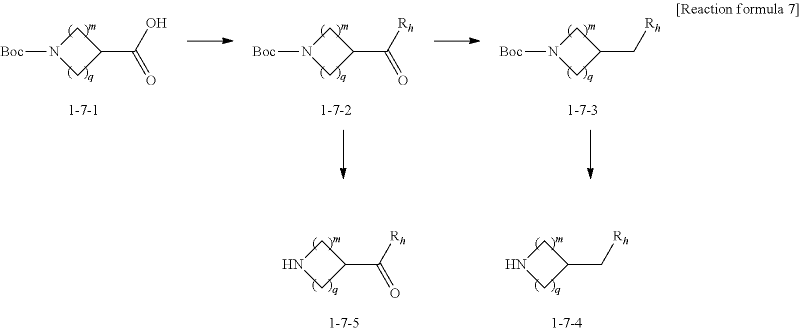

According to above reaction formula 7, amide may be introduced into a compound of formula 1-7-1 to prepare a compound of formula 1-7-2, which may be then subjected to a reduction reaction to prepare a compound of formula 1-7-3. A protecting group may be removed from the compound of formula 1-7-3, so as to prepare a compound of formula 1-7-4 as a compound of formula 1-1-1 (R₅—H). And, a protecting group may be removed from the compound of formula 1-7-2, so as to prepare a compound of formula 1-7-5 as a compound of formula 1-1-1 (R₅—H).

In the present invention, examples of compounds which may be synthesized according to a method of above reaction formula 1 or 2 using R₅—H prepared by the same method as shown in above reaction formula 7 may include example compounds 131 to 133, 331, 402, 406 or the like.

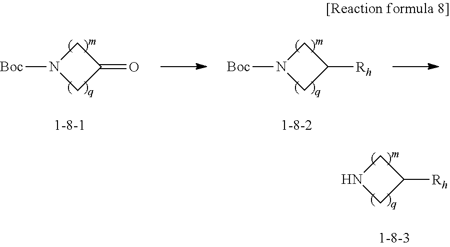

According to above reaction formula 8, a compound of formula 1-8-2 may be prepared through a reductive amination reaction to a compound of formula 1-8-1. A protecting group (Boc) may be removed from a compound of formula 1-8-2, so as to prepare a compound of formula 1-8-3.

In the present invention, examples of compounds which may be synthesized according to a method of above reaction formula 1 or 2 using R₅—H prepared by the same method as shown in above reaction formula 8 may include example compounds 186, 390, 391, 400, 405 or the like.

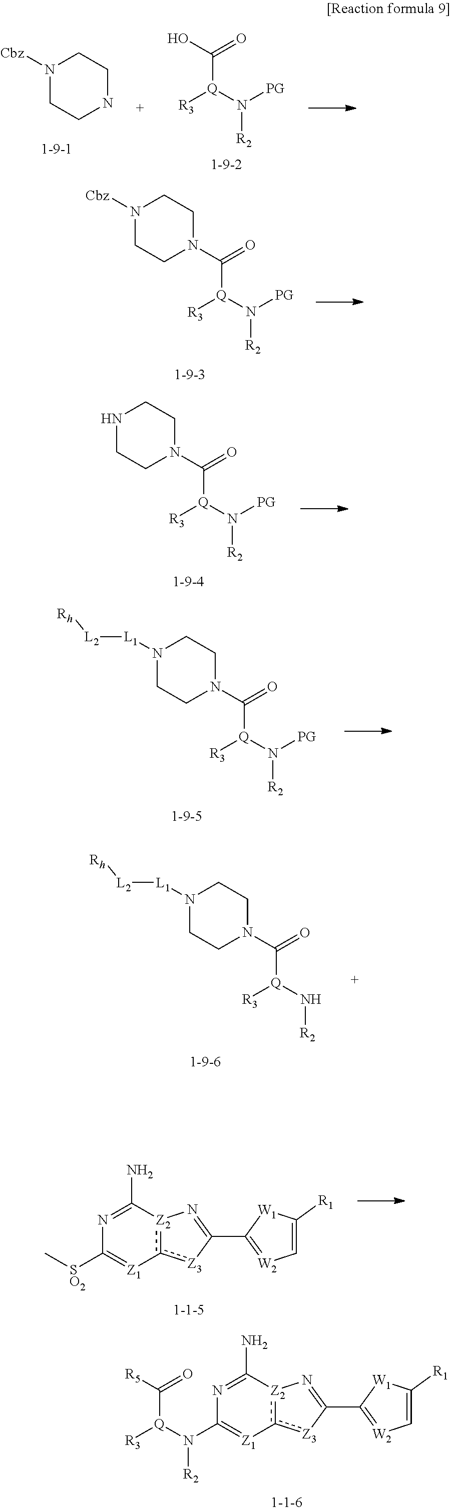

In above reaction formula 9, a compound of formula 1-9-1 and formula 1-9-2 may be subjected to a reaction to prepare a compound of formula 1-9-3, after which a protecting group (Cbz) may be removed from N of piperazine, so as to prepare a compound of formula 1-9-4. A substituent may be introduced into the compound of formula 1-9-4 to prepare a compound of formula 1-9-5, after which a protecting group (PG) may be removed from N of amine, so as to prepare a compound of formula 1-9-6. After that, a compound of formula 1-1-6 may be prepared through a substitution reaction with a compound of formula 1-1-5.

In the present invention, the compounds prepared by above reaction formula 9 may include example compounds 4 to 6, 17, 18, 21 to 25, 45 to 47, 61 to 64, 66, 68 to 72, 82, 89, 99 to 106, 111, 115 to 117, etc.

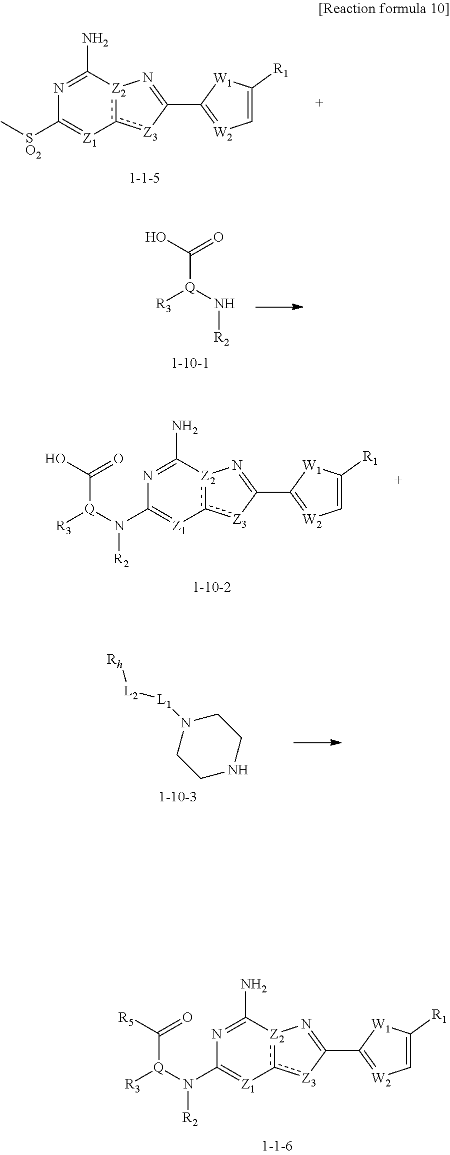

In above reaction formula 10, a compound of formula 1-10-1 may be introduced into a compound of formula 1-1-5 to obtain a compound of formula 1-10-2, after which the compound of formula 1-10-2 and a compound of formula 1-10-3 may be subjected to a reaction, thereby preparing a compound of formula 1-1-6.

In the present invention, the compounds prepared by above reaction formula 10 may include example compounds 53, 54, etc.

Composition Including Compound Represented by Formula 1, Use Thereof and Therapeutic Method Using the Same

The present invention may provide a pharmaceutical composition including a compound represented by above formula 1, compounds exemplified in this specification, stereoisomers thereof or pharmaceutically acceptable salts thereof as an active ingredient.

In addition, the present invention may provide a pharmaceutical composition for treating or preventing A2a receptor-associated diseases, including a compound represented by above formula 1, compounds exemplified in this specification, stereoisomers thereof or pharmaceutically acceptable salts thereof as an active ingredient.

The A2a receptor-associated diseases may be cancer or inflammatory diseases.

The cancer may be at least one selected from lung cancer, stomach cancer, ovarian cancer, prostate cancer, esophageal cancer, gastrointestinal cancer, pancreatic cancer, colorectal cancer, kidney cancer, testicular cancer, bladder cancer, breast cancer, uterine cancer, cervical cancer, head and neck cancer, blood cancer, bone cancer, liver cancer, thyroid cancer, skin cancer, lymphoma, leukemia, myeloma, sarcoma and virus-associated cancer.

The inflammatory disease may be at least one selected from rheumatoid arthritis, multiple sclerosis, Crohn's disease, ulcerative colitis, graft-versus-host disease, systemic lupus erythematosus, toxic shock syndrome, osteoarthritis, and insulin-dependent diabetes.

For administration, a pharmaceutical composition of the present invention may further include at least one type of a pharmaceutically acceptable carrier, in addition to the compound represented by above formula 1, stereoisomers thereof or pharmaceutically acceptable salts thereof. The pharmaceutically acceptable carrier to be used herein may include saline solution, sterilized water, Ringer's solution, buffered saline, dextrose solution, maltodextrin solution, glycerol, ethanol and a mixture of at least one ingredient thereof, and with the addition of other conventional additives such as antioxidants, buffer solutions, bacteriostatic agents, etc., if needed. In addition, diluents, dispersing agents, surfactants, binders and lubricants may be further added to formulate injectable dosage forms such as aqueous solutions, suspensions, emulsions, etc., pills, capsules, granules or tablets. Thus, the composition of the present invention may be patches, liquid medicines, pills, capsules, granules, tablets, suppositories, etc. The preparations may be prepared according to a conventional method used for formulation in the art or a method disclosed in Remington's Pharmaceutical Science (latest edition), Mack Publishing Company, Easton PA, and the composition may be formulated into various preparations depending on each disease or ingredient.

The composition of the present invention may be orally or parenterally administered (for example, applied intravenously, hypodermically, intraperitoneally or locally) according to a targeted method, in which a dosage thereof may vary in a range thereof depending on a patient's weight, age, gender, health condition and diet, an administration time, an administration method, an excretion rate, a severity of a disease and the like. The compound represented by formula 1 of the present invention may be administered once or several times a day by dividing the daily dosage of the compound, but is not necessarily limited thereto.

In addition to the compound represented by above formula 1, the compound exemplified in this specification, stereoisomers thereof or pharmaceutically acceptable salts thereof, the pharmaceutical composition of the present invention may further include at least one ingredient which may exhibit the same or similar medicinal effects or may bring synergy to medicinal effects in combination.

The present invention may provide a method for treating or preventing adenosine A2a receptor-associated diseases, including administering a therapeutically effective amount of the compound represented by above formula 1, the compound exemplified in this specification, stereoisomers thereof or pharmaceutically acceptable salts thereof; or a pharmaceutical composition including the same as an effective ingredient into a subject in need thereof.

As used herein, the term “therapeutically effective amount” may refer to an amount of the compound, the compound exemplified in this specification, stereoisomers thereof or pharmaceutically acceptable salts thereof, which are effective in treating or preventing adenosine A2a receptor-associated diseases. The adenosine A2a receptor-associated diseases may be cancer or inflammatory diseases.

In the present invention, the term “subject” may refer to mammals including humans, and the term “administration” may refer to providing a predetermined material to a subject through any appropriate method. It is apparent to those skilled in the art that the therapeutically effective dosage and the number of administration for effective ingredient of the present invention may vary depending on a desired effect.

In the present invention, the term “prevention” may refer to a delay of occurrence of disease, disorder or condition. If the occurrence of disease, disorder or condition is delayed for an expected period of time, the prevention may be considered as complete.

In the present invention, the term “treatment” may refer to the one that partially or completely reduces, ameliorates, alleviates, inhibits or delays the occurrence of a certain disease, disorder and/or condition, reduces a severity thereof, or reduces the occurrence of at least one symptom or property thereof.

The present invention may also provide a use of the compound represented by formula 1, the compound exemplified in this specification, stereoisomers thereof or pharmaceutically acceptable salts thereof; or a pharmaceutical composition including the same as an effective ingredient for treating or preventing adenosine A2a receptor-associated diseases. The adenosine A2a receptor-associated diseases may be cancer or inflammatory diseases.

The present invention may also provide a use of the compound represented by formula 1, the compound exemplified in this specification, stereoisomers thereof or pharmaceutically acceptable salts thereof; or a pharmaceutical composition including the same as an effective ingredient in preparing a medicament for treating or preventing adenosine A2a receptor-associated diseases. The adenosine A2a receptor-associated diseases may be cancer or inflammatory diseases.

Matters mentioned in the composition, therapeutic method and use of the present invention are equally applied, if not contradictory to each other.

### Advantageous Effects of Invention

A compound of the present invention, stereoisomers thereof or pharmaceutically acceptable salts thereof can exhibit an effective antagonistic activity against adenosine A2a receptors and can be advantageously used for treatment or prevention of adenosine A2a receptor-associated diseases.

### MODE FOR INVENTION

Hereinafter, the present invention will be described in more detail through preparation examples and exemplary examples. However, the following preparation examples and exemplary examples are provided for the purpose of illustrating the present invention, and thus the present invention is not limited to the preparation examples and exemplary examples.

### Preparation of Compound Represented by Formula 1

Each of the compounds according to the present invention was synthesized as follows.

In order to prepare compounds according to the present invention, each of the reaction compounds used in each reaction was purchased from Sigma Aldrich (company name), etc., or was synthesized by using an organic synthesis method obvious to those skilled in the chemistry field, and was used without a separate purification process. The compounds of each example were identified through 1H-NMR (Bruker, avance II 400) and Mass (Waters, SQD2) analysis.

### Example 1: Synthesis of Compound 1, (S)-2-((7-amino-2-(furan-2-yl)-[1,2,4]triazolo[1,5-a][1,3,5]triazin-5-yl)amino)-1-(4-methylpiperazin-1-yl)-3-phenylpropan-1-one

### [Step 1] Synthesis of tert-butyl (S)-(1-(4-methylpiperazin-1-yl)-1-oxo-3-phenylpropan-2-yl)carbamate

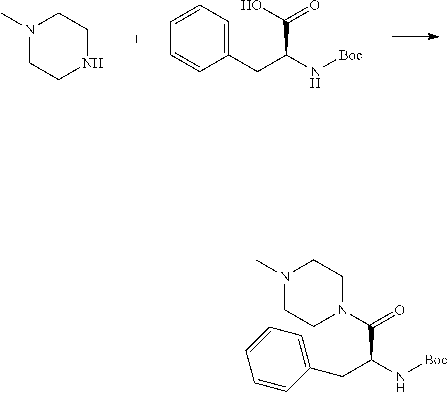

(Tert-butoxycarbonyl)-L-phenylalanine (10.000 g, 37.692 mmol), 1-methylpiperazine (8.390 mL, 75.384 mmol), [dimethylamino(triazolo[4,5-b]pyridin-3-yloxy)methylidene]-dimethylazanium, hexafluorophosphate (28.664 g, 75.384 mmol) and N,N-diisopropylethylamine (13.130 mL, 75.384 mmol) were dissolved in N,N-dimethylformamide (200 mL) at room temperature, and the resulting solution was stirred at the same temperature for 18 hours. Solvent was removed from the reaction mixture under reduced pressure, after which water was poured into the resulting concentrate and an organic layer was extracted with ethyl acetate. The organic layer was washed with saturated aqueous solution of sodium chloride, dehydrated with anhydrous sodium sulfate, filtered, and concentrated under reduced pressure. The resulting concentrate was purified via column chromatography (SiO₂, 120 g cartridge; methanol/dichloromethane=0 to 10%) and concentrated to obtain a title compound (10.000 g, 76.4%) as a white solid of a foam type.

### [Step 2] Synthesis of (S)-2-amino-1-(4-methylpiperazin-1-yl)-3-phenylpropan-1-one

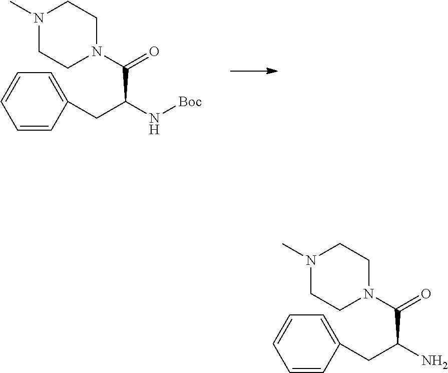 [SMILES: CN1CCN(C(=O)[C@H](Cc2ccccc2)NC(=O)OC(C)(C)C)CC1 ; CN1CCN(C(=O)[C@@H](N)Cc2ccccc2)CC1]

Tert-butyl (S)-(1-(4-methylpiperazin-1-yl)-1-oxo-3-phenylpropan-2-yl)carbamate (9.000 g, 25.902 mmol) prepared in step 1 and 2,2,2-trifluoroacetic acid (9.911 mL, 129.511 mmol) were dissolved in methanol (100 mL) at room temperature, after which the resulting solution was stirred at the same temperature for 18 hours. Aqueous solution of 2N-sodium hydroxide was poured into the reaction mixture and an organic layer was extracted with ethyl acetate. The organic layer was washed with saturated aqueous solution of sodium chloride, dehydrated with anhydrous sodium sulfate, filtered, and concentrated under reduced pressure. An obtained product was used without an additional purification process (title compound, 3.500 g, 54.6%, white solid).

### [Step 3] Synthesis of (S)-2-((7-amino-2-(furan-2-yl)-[1,2,4]triazolo[1,5-a][1,3,5]triazin-5-yl)amino)-1-(4-methylpiperazin-1-yl)-3-phenylpropan-1-one

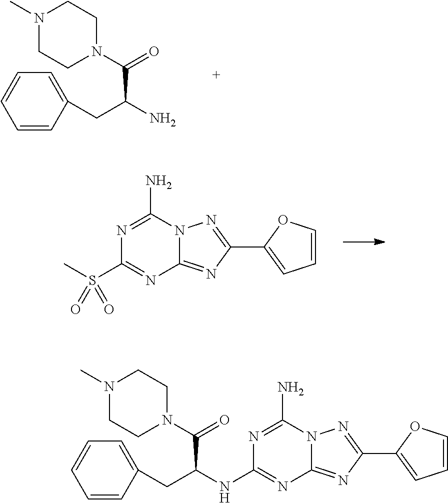 [SMILES: CN1CCN(C(=O)[C@@H](N)Cc2ccccc2)CC1 ; CS(=O)(=O)c1nc(N)n2nc(-c3ccco3)nc2n1 ; CN1CCN(C(=O)[C@H](Cc2ccccc2)Nc2nc(N)n3nc(-c4ccco4)nc3n2)CC1]

2-(Furan-2-yl)-5-(methylsulfonyl)-[1,2,4]triazolo[1,5-a][1,3,5]triazine-7-amine (3.500 g, 12.488 mmol) prepared in step 2, (S)-2-amino-1-(4-methylpiperazin-1-yl)-3-phenylpropan-1-one (3.089 g, 12.488 mmol) and triethylamine (3.481 mL, 24.977 mmol) were dissolved in dimethylsulfoxide (60 mL) at room temperature, and the resulting solution was stirred at the same temperature for 18 hours. Water was poured into the reaction mixture and an organic layer was extracted with ethyl acetate. The organic layer was washed with water, dehydrated with anhydrous magnesium sulfate, filtered, and concentrated under reduced pressure. The resulting concentrate was purified via column chromatography (SiO₂, 12 g cartridge; methanol/dichloromethane=0 to 8%) and concentrated to obtain a title compound (3.000 g, 53.7%) as a white solid form.

¹H NMR (400 MHz, Chloroform-d) δ 9.34 (s, 1H), 8.47-8.36 (m, 1H), 7.62 (s, 1H), 7.31-7.08 (m, 6H), 6.59 (s, 1H), 6.30-6.25 (m, 1H), 5.72-5.65 (m, 1H), 3.81-3.38 (m, 4H), 3.11-3.05 (m, 2H), 2.39-2.31 (m, 2H), 2.21 (s, 3H), 2.17-2.13 (m, 1H), 1.83-1.79 (m, 1H), 1.33 (s, 1H), 1.28 (s, 1H); LRMS (ES) m/z 488.4

### Examples 2, 3, 13, 19, 20, 28 and 191

Example compounds 2, 3, 13, 19, 20, 28 and 191 were each prepared through substantially the same synthesis method as a synthesis method of example compound 1 except for using the compounds of the following table instead of 1-methylpiperazine as R₅—H of above reaction formula 1 in step 1 of a synthesis method of example 1.

TABLE 2

| Example | |
|---|---|
| No. | R₅—H |
| 2 | 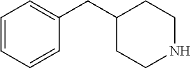 |
| 3 | 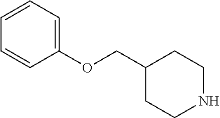 |
| 13 | 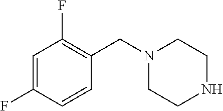 |
| 19 | 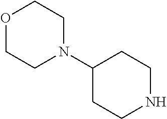 |
| 20 | 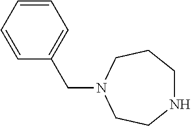 |
| 28 | 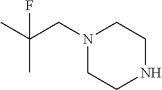 |
| 191 | 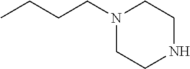 |

### Example 26: Synthesis of Compound 26, (S)-(1-(7-amino-2-(furan-2-yl)-[1,2,4]triazolo[1,5-a][1,3,5]triazin-5-yl)pyrrolidin-2-yl)(4-(2-fluoro-2-methylpropyl)piperazin-1-yl)methanone

### [Step 1] Synthesis of tert-butyl (S)-2-(4-(2-fluoro-2-methylpropyl)piperazin-1-carbonyl)pyrrolidin-1-carboxylate

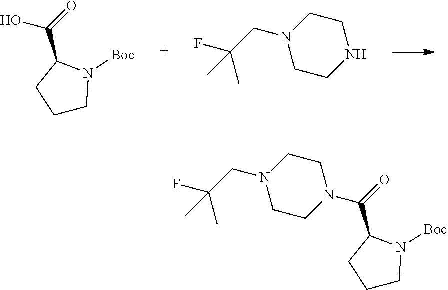 [SMILES: CC(C)(C)OC(=O)N1CCC[C@H]1C(=O)O ; CC(C)(F)CN1CCNCC1 ; CC(C)(F)CN1CCN(C(=O)[C@@H]2CCCN2C(=O)OC(C)(C)C)CC1]

(Tert-butoxycarbonyl)-L-proline (2.000 g, 9.292 mmol), 1-(2-fluoro-2-methylpropyl)piperazine (2.978 g, 18.583 mmol), [dimethylamino(triazolo[4,5-b]pyridin-3-yloxy)methylidene]-dimethylazanium; hexafluorophosphate (7.066 g, 18.583 mmol) and N,N-diisopropylethylamine (6.474 mL, 37.166 mmol) were dissolved in N,N-dimethylformamide (20 mL) at room temperature, and the resulting solution was stirred at the same temperature for 18 hours. Solvent was removed from the reaction mixture under reduced pressure, after which saturated aqueous solution of ammonium chloride was poured into the resulting concentrate and an organic layer was extracted with ethyl acetate. The organic layer was washed with saturated aqueous solution of sodium hydrogen carbonate, dehydrated with anhydrous sodium sulfate, filtered, and concentrated under reduced pressure. The resulting concentrate was purified via column chromatography (SiO₂, 24 g cartridge; ethyl acetate/hexane=0 to 50%) and concentrated to obtain a title compound (2.000 g, 60.2%) as a yellow solid form.

### [Step 2] Synthesis of (S)-1-(2-fluoro-2-methylpropyl)-4-prolylpiperazine

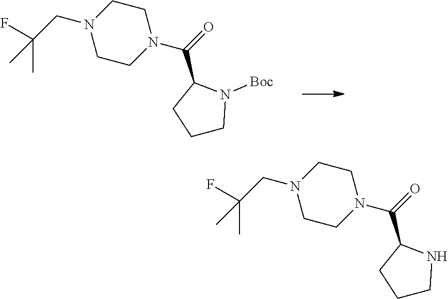 [SMILES: CC(C)(F)CN1CCN(C(=O)[C@@H]2CCCN2C(=O)OC(C)(C)C)CC1 ; CC(C)(F)CN1CCN(C(=O)[C@@H]2CCCN2)CC1]

Tert-butyl (S)-2-(4-(2-fluoro-2-methylpropyl)piperazin-1-carbonyl)pyrrolidin-1-carboxylate (0.200 g, 0.559 mmol) prepared in step 1 and hydrochloric acid (4.00 M solution in dioxane, 0.699 mL, 2.797 mmol) were dissolved in dichloromethane (5 mL)/methanol (1 mL) at room temperature, after which the resulting solution was stirred at the same temperature for 18 hours. Solvent was removed from the reaction mixture under reduced pressure, after which an obtained product was used without an additional purification process (title compound, 0.140 g, 97.2%, brown solid).

### [Step 3] Synthesis of (S)-(1-(7-amino-2-(furan-2-yl)-[1,2,4]triazolo[1,5-a][1,3,5]triazin-5-yl)pyrrolidin-2-yl)(4-(2-fluoro-2-methylpropyl)piperazin-1-yl)methanone

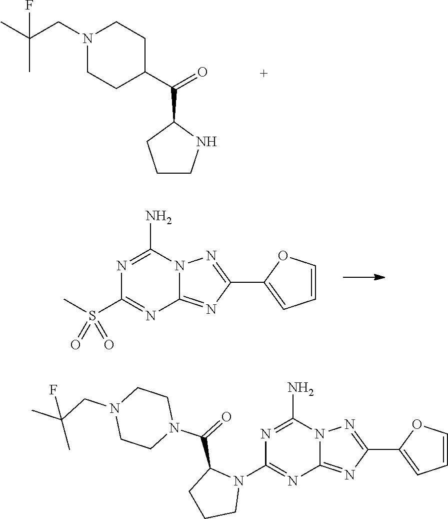

2-(furan-2-yl)-5-(methylsulfonyl)-[1,2,4]triazolo[1,5-a][1,3,5]triazine-7-amine (0.050 g, 0.178 mmol) prepared in step 2, (S)-1-(2-fluoro-2-methylpropyl)-4-prolylpiperazine (0.046 g, 0.178 mmol) and triethylamine (0.050 mL, 0.357 mmol) were dissolved in dimethylsulfoxide (1 mL) at room temperature, after which the resulting solution was stirred for 18 hours at the same temperature. Water was poured into the reaction mixture, and then an organic layer was extracted with dichloromethane, filtered via a plastic filter to remove a solid residue and an aqueous solution layer therefrom, and concentrated under reduced pressure. The resulting concentrate was purified via chromatography (SiO₂ plate, 20×20×1 mm; methanol/dichloromethane=0 to 5%) and concentrated to obtain a title compound (0.013 g, 15.3%) as a brown solid form.

¹H NMR (400 MHz, Chloroform-d) δ 7.60-7.51 (m, 1H), 7.24-7.13 (m, 1H), 6.54 (ddd, J=11.3, 3.4, 1.8 Hz, 1H), 6.30 (s, 1H), 6.14 (s, 1H), 5.13-4.83 (m, 1H), 3.95-3.39 (m, 6H), 2.72 (s, 1H), 2.57-2.38 (m, 4H), 2.29 (ddd, J=13.8, 9.7, 5.3 Hz, 1H), 2.16 (dt, J=12.7, 7.7 Hz, 1H), 1.47-1.33 (m, 6H). LRMS (ES) m/z 458.4 (M⁺+1).

### Example 48: Synthesis of Compound 48, (S)-(1-(7-amino-2-(furan-2-yl)-[1,2,4]triazolo[1,5-a][1,3,5]triazin-5-yl)pyrrolidin-2-yl)(4-(2,2,2-trifluoroethyl)piperazin-1-yl)methanone

### [Step 1] Synthesis of tert-butyl (S)-2-(4-(2,2,2-trifluoroethyl)piperazin-1-carbonyl)pyrrolidin-1-carboxylate

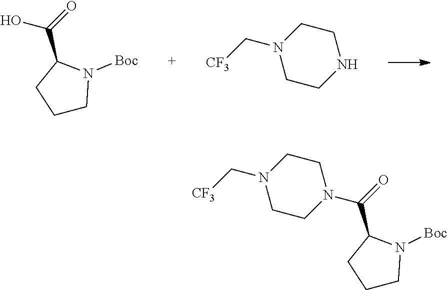 [SMILES: CC(C)(C)OC(=O)N1CCC[C@H]1C(=O)O ; FC(F)(F)CN1CCNCC1 ; CC(C)(C)OC(=O)N1CCC[C@H]1C(=O)N1CCN(CC(F)(F)F)CC1]

(Tert-butoxycarbonyl)-L-proline (1.000 g, 4.646 mmol), 1-(2,2,2-trifluoroethyl)piperazine (0.781 g, 4.646 mmol), [dimethylamino(triazolo[4,5-b]pyridin-3-yloxy)methylidene]-dimethylazanium; hexafluorophosphate (3.533 g, 9.292 mmol) and N,N-diisopropylethylamine (1.618 mL, 9.292 mmol) were dissolved in N,N-dimethylformamide (20 mL) at room temperature, and the resulting solution was stirred at the same temperature for 18 hours. Water was poured into the reaction mixture and an organic layer was extracted with ethyl acetate. The organic layer was washed with saturated aqueous solution of sodium chloride, dehydrated with anhydrous magnesium sulfate, filtered, and concentrated under reduced pressure. The resulting concentrate was purified via column chromatography (SiO₂, 12 g cartridge; ethyl acetate/hexane=0 to 50%) and concentrated to obtain a title compound (0.700 g, 41.2%) as a yellow solid form.

### [Step 2] Synthesis of (S)-1-prolyl-4-(2,2,2-trifluoroethyl)piperazine

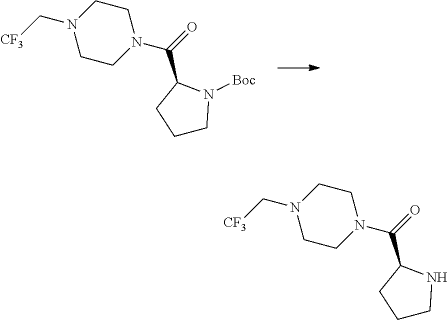 [SMILES: CC(C)(C)OC(=O)N1CCC[C@H]1C(=O)N1CCN(CC(F)(F)F)CC1 ; O=C([C@@H]1CCCN1)N1CCN(CC(F)(F)F)CC1]

Tert-butyl (S)-2-(4-(2,2,2-trifluoroethyl)piperazine-1-carbonyl)pyrrolidin-1-carboxylate (0.200 g, 0.547 mmol) prepared in step 1 and hydrochloric acid (4.00 M solution dioxane, 1.368 mL, 5.473 mmol) were dissolved in dichloromethane (5 mL) at room temperature, after which the resulting solution was stirred at the same temperature for 18 hours. Solvent was removed from the reaction mixture under reduced pressure, after which an obtained product was used without an additional purification process (title compound, 0.140 g, 96.4%, white solid).

### [Step 3] Synthesis of (S)-(1-(7-amino-2-(furan-2-yl)-[1,2,4]triazolo[1,5-a][1,3,5]triazin-5-yl)pyrrolidin-2-yl)(4-(2,2,2-trifluoroethyl)piperazin-1-yl)methanone

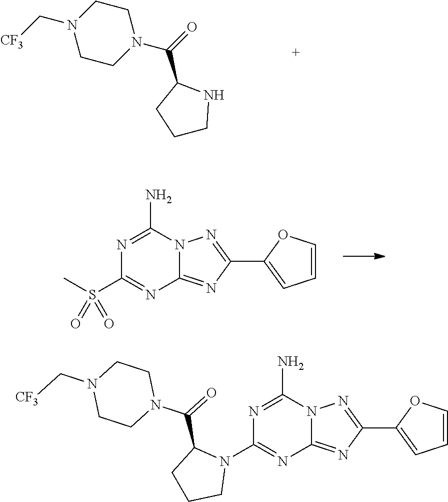

(S)-1-Propyl-4-(2,2,2-trifluoroethyl)piperazine (0.050 g, 0.188 mmol) prepared in step 2, 2-(furan-2-yl)-5-(methylsulfonyl)-[1,2,4]triazolo[1,5-a][1,3,5]triazin-7-amine (0.053 g, 0.188 mmol) and triethylamine (0.053 mL, 0.377 mmol) were dissolved in dimethylsulfoxide (1 mL) at room temperature, after which the resulting solution was stirred for 18 hours at the same temperature. Water was poured into the reaction mixture, and then an organic layer was extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; methanol/dichloromethane=0 to 10%) and concentrated to obtain a title compound (0.015 g, 16.5%) as a white solid form.

¹H NMR (400 MHz, Chloroform-d) δ 7.61-7.54 (m, 1H), 7.27-7.13 (m, 1H), 6.56 (ddd, J=5.2, 3.4, 1.8 Hz, 1H), 5.95 (d, J=74.2 Hz, 2H), 5.02 (ddd, J=63.5, 8.5, 3.0 Hz, 1H), 3.99-3.71 (m, 4H), 3.67-3.61 (m, 2H), 3.09 (p, J=9.5, 9.1 Hz, 3H), 2.80-2.60 (m, 3H), 2.38-2.08 (m, 2H), 2.06-1.97 (m, 2H). LRMS (ES) m/z 466.5 (M⁺+1).

### Example 90: Synthesis of Compound 90, (S)-(1-(7-amino-2-(5-methylfuran-2-yl)-[1,2,4]triazolo[1,5-a][1,3,5]triazin-5-yl)pyrrolidin-2-yl)(4-(2-fluoro-2-methylpropyl)piperazin-1-yl)methanone

### [Step 1] Synthesis of (S)-(1-(7-amino-2-(5-methylfuran-2-yl)-[1,2,4]triazolo[1,5-a][1,3,5]triazin-5-yl)pyrrolidin-2-yl)(4-(2-fluoro-2-methylpropyl)piperazin-1-yl)methanone

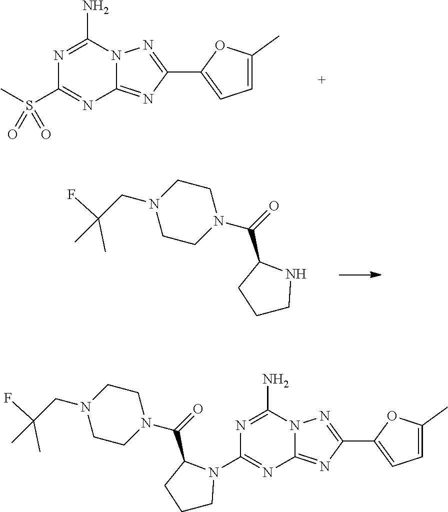

(S)-1-(2-Fluoro-2-methylpropyl)-4-prolylpiperazine (0.087 g, 0.340 mmol) prepared in synthesis step 2 of Example 26, 2-(5-methylfuran-2-yl)-5-(methylsulfonyl)-[1,2,4]triazolo[1,5-a][1,3,5]triazin-7-amine (0.050 g, 0.170 mmol) and triethylamine (0.047 mL, 0.340 mmol) were dissolved in dimethylsulfoxide (5 mL) at room temperature, and the resulting solution was stirred at the same temperature for 18 hours. Water was poured into the reaction mixture and an organic layer was extracted with ethyl acetate. The organic layer was washed with water, dehydrated with anhydrous sodium sulfate, filtered, and concentrated under reduced pressure. The resulting concentrate was purified via chromatography (SiO₂ plate, 20×20×1 mm; methanol/dichloromethane=5%) and concentrated to obtain a title compound (0.035 g, 43.7%) as a white solid form.

¹H NMR (400 MHz, DMSO-d₆) δ 8.27 (m, 2H), 6.94 (ddd, J=3.2, 2.2, 0.6 Hz, 1H), 6.29 (ddd, J=3.3, 1.7, 1.1 Hz, 1H), 5.02-4.92 (m, 1H), 3.72-3.39 (m, 6H), 2.72 (m, 1H), 2.61-2.52 (m, 5H), 2.48-2.18 (m, 4H), 2.04-1.71 (m, 3H), 1.39-1.26 (m, 6H); LRMS (ES) m/z 472.6 (M⁺+1).

### Examples 12, 16, 27, 55, 78 to 81, 113, 114, 120 to 128, 134 to 142, 145, 146, 149, 150, 151, 236, 239, 246, 247, 265, 266, 281, 301, 302, 309, 310 and 321

Example compounds 12, 16, 27, 55, 78 to 81, 113, 114, 120 to 128, 134 to 142, 145, 146, 149, 150, 151, 236, 239, 246, 247, 265, 266, 281, 301, 302, 309, 310 and 321 were each prepared through substantially the same synthesis method as a synthesis method of example compound 26 except for using the compounds of the following table instead of 1-(2-fluoro-2-methylpropyl)piperazine as R₅—H of above reaction formula 1 in step 1 of a synthesis method of example compound 26.

TABLE 3

| Example | |
|---|---|
| No. | R₅-H |
| 12 | 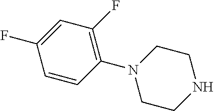 |
| 16 | 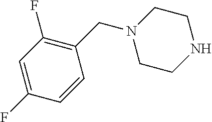 |
| 27 | 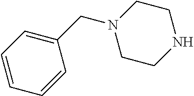 |
| 55 | 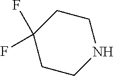 |
| 78 | 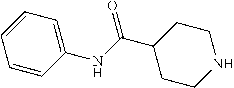 |
| 79 | 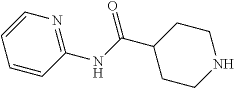 |
| 80 | 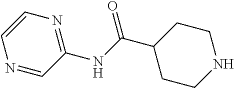 |
| 81 | 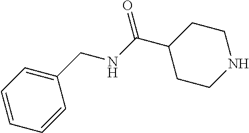 |
| 113 | 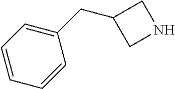 |
| 114 | 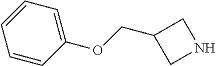 |
| 120 | 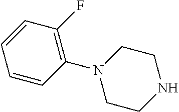 |
| 121 | 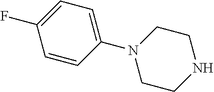 |
| 122 | 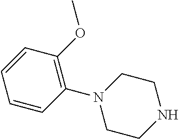 |
| 123 | 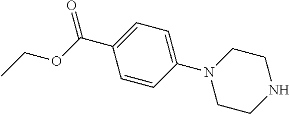 |
| 124 | 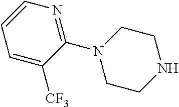 |
| 125 | 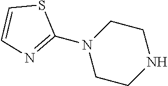 |
| 126 | 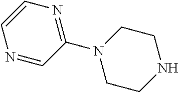 |
| 127 | 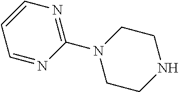 |
| 128 | 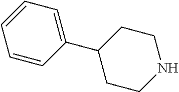 |
| 134 | 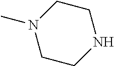 |
| 135 | 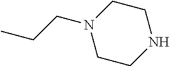 |
| 136 | 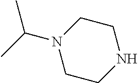 |
| 137 | 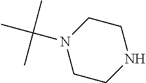 |
| 138 | 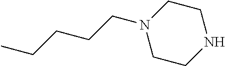 |
| 139 | 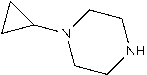 |
| 140 | 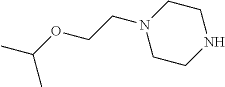 |
| 141 | 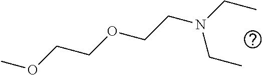 |
| 142 | 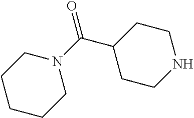 |
| 145 | 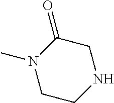 |
| 146 | 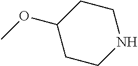 |
| 149 | 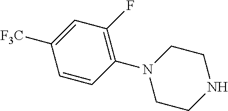 |
| 150 | 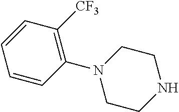 |
| 151 | 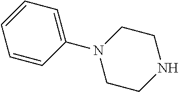 |
| 236 | 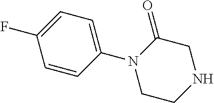 |
| 239 | 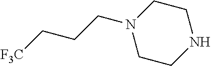 |
| 246 | 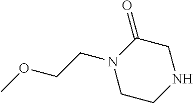 |
| 247 | 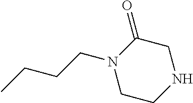 |
| 281 | 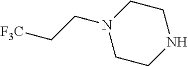 |
| 301 | 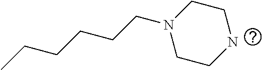 |
| 302 | 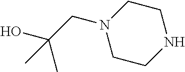 |
| 309 | 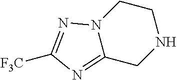 |
| 310 | 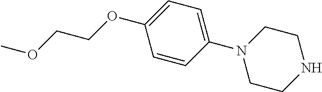 |
| 321 | 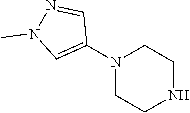 |

### Example 15: Synthesis of Compound 15

Example compound 15 was synthesized through substantially the same synthesis method as a synthesis method of example compound 16 except for using (tert-butoxycarbonyl)glycine instead of (tert-butoxycarbonyl)-L-proline.

### Example 37: Synthesis of Compound 37

Example compound 37 was synthesized through substantially the same synthesis method as a synthesis method of example compound 12 except for using (2S,4R)-1-(tert-butoxycarbonyl)-4-hydroxypyrrolidin-2-carboxylic acid instead of (tert-butoxycarbonyl)-L-proline.

### Example 385: Synthesis of Compound 385

Example compound 385 was synthesized through substantially the same synthesis method as a synthesis method of example compound 48 except for using (S)-5-(tert-butoxycarbonyl)-5-azaspiro[2.4]heptan-6-carboxylic acid instead of (tert-butoxycarbonyl)-L-proline.

### Example 386: Synthesis of Compound 386

Example compound 386 was synthesized through substantially the same synthesis method as a synthesis method of example compound 26 except for using (S)-5-(tert-butoxycarbonyl)-5-azaspiro[2.4]heptan-6-carboxylic acid instead of (tert-butoxycarbonyl)-L-proline.

### Example 387: Synthesis of Compound 387

Example compound 387 was synthesized through substantially the same synthesis method as a synthesis method of example compound 48 except for using (S)-1-(tert-butoxycarbonyl)-4,4-dimethylpyrrolidin-2-carboxylic acid instead of (tert-butoxycarbonyl)-L-proline.

### Example 388: Synthesis of Compound 388

Example compound 388 was synthesized through substantially the same synthesis method as a synthesis method of example compound 26 except for using (tert-butoxycarbonyl)glycine instead of (tert-butoxycarbonyl)-L-proline.

### Examples 65, 107, 108, 109, 110, 112, 242, 264 and 350

Example compound 108 was synthesized through substantially the same synthesis method as a synthesis method of example compound 26 except for using (S)-1-(tert-butoxycarbonyl)azetidin-2-carboxylic acid instead of (tert-butoxycarbonyl)-L-proline.

In addition, example compounds 65, 107, 109, 110, 112, 242, 264 and 350 were each prepared through substantially the same synthesis method as a synthesis method of example compound 26 except for using the compounds of the following table instead of 1-(2-fluoro-2-methylpropyl)piperazine as R₅—H of reaction formula 1.

TABLE 4

| Example | |
|---|---|
| No. | R₅—H |
| 65 | 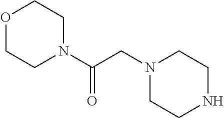 |
| 107 | 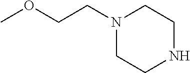 |
| 109 | 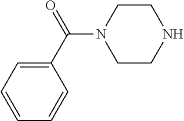 |
| 110 | 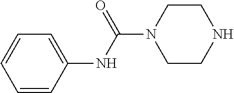 |
| 112 | 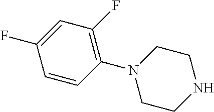 |
| 242 | 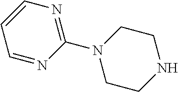 |
| 264 | 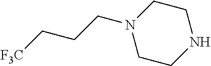 |
| 350 | 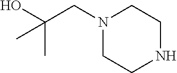 |

### Example 29: Synthesis of Compound 29

Example compound 29 was synthesized through substantially the same synthesis method as a synthesis method of example compound 16 except for using (S)-1-(tert-butoxycarbonyl)piperidin-2-carboxylic acid instead of (tert-butoxycarbonyl)-L-proline.

### Examples 92, 93, 94, 95, 96, 97 and 98

Example compounds 92, 93, 94, 95, 96, 97 and 98 were prepared through substantially the same synthesis method as a synthesis method of example compound 26 except for using the compounds of the following table instead of 1-(2-fluoro-2-methylpropyl)piperazine as R₅—H of reaction formula 1 and using tert-butoxycarbonyl-D-proline instead of (tert-butoxycarbonyl)-L-proline.

TABLE 5

| Example | |
|---|---|
| No. | R₅—H |
| 92 | 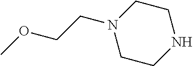 |
| 93 | 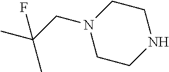 |
| 94 | 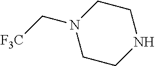 |
| 95 | 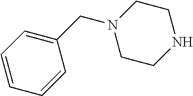 |
| 96 | 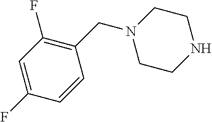 |
| 97 | 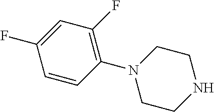 |
| 98 | 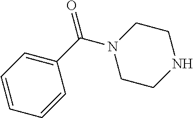 |

### Example 7: Synthesis of Compound 7

Example compound 7 was synthesized through substantially the same synthesis method as a synthesis method of example compound 12 except for using (S)-2-((tert-butoxycarbonyl)amino)-2-phenylacetic acid instead of (tert-butoxycarbonyl)-L-proline.

### Example 14: Synthesis of Compound 14

Example compound 14 was synthesized through substantially the same synthesis method as a synthesis method of example compound 16 except for using (S)-2-((tert-butoxycarbonyl)amino)-2-phenylacetic acid instead of (tert-butoxycarbonyl)-L-proline.

### Example 190: Synthesis of Compound 190

Example compound 190 was synthesized through substantially the same synthesis method as a synthesis method of example compound 191 except for using (S)-2-((tert-butoxycarbonyl)amino)-2-phenylacetic acid instead of (tert-butoxycarbonyl)-L-phenylalanine.

### Example 195: Synthesis of Compound 195

Example compound 195 was synthesized through substantially the same synthesis method as a synthesis method of example compound 26 except for using (S)-2-((tert-butoxycarbonyl)amino)-2-phenylacetic acid instead of (tert-butoxycarbonyl)-L-proline.

### Examples 171, 241 and 249

Example compound 171 was synthesized through substantially the same synthesis method as a synthesis method of example compound 12 except for using (S)-2-((tert-butoxycarbonyl)amino)-2-(tetrahydro-2H-pyran-4-yl)acetic acid instead of (S)-2-((tert-butoxycarbonyl)amino)-2-phenylacetic acid.

In addition, example compounds 241 and 249 were each prepared through substantially the same synthesis method as a synthesis method of example compound 171 except for using the compounds of the following table instead of 1-(2,4-difluorophenyl)piperazine as R₅—H.

TABLE 6

| Example | |
|---|---|
| No. | R₅—H |
| 241 | 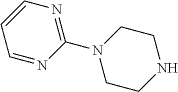 |
| 249 | 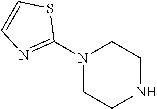 |

### Examples 8 and 231 to 235

Example compound 8 was synthesized through substantially the same synthesis method as a synthesis method of example compound 12 except for using (tert-butoxycarbonyl)-L-serine instead of (S)-2-((tert-butoxycarbonyl)amino)-2-phenylacetic acid.

In addition, example compounds 231 to 235 were each prepared through substantially the same synthesis method as a synthesis method of example compound 8 except for using the compounds of the following table instead of 1-(2,4-difluorophenyl)piperazine as R₅—H.

TABLE 7

| Example | |
|---|---|
| No. | R₅—H |
| 8 | 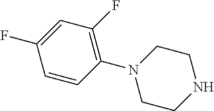 |
| 231 | 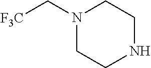 |
| 232 | 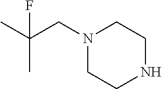 |
| 233 | 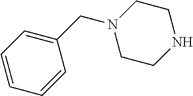 |
| 234 | 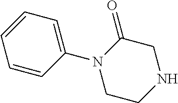 |
| 235 | 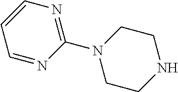 |

### Examples 9, 118, 119, 240, 251 and 349

Example compound 9 was synthesized through substantially the same synthesis method as a synthesis method of example compound 8 except for using (tert-butoxycarbonyl)-L-alanine instead of (tert-butoxycarbonyl)-L-serine.

In addition, example compounds 118, 119, 240, 251 and 349 were each prepared through substantially the same synthesis method as a synthesis method of example compound 9 except for using the compounds of the following table instead of 1-(2,4-difluorophenyl)piperazine as R₅—H.

TABLE 8

| Example | |
|---|---|
| No. | R₅—H |
| 118 | 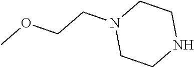 |
| 119 | 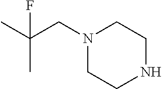 |
| 240 | 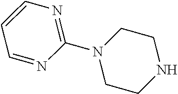 |
| 251 | 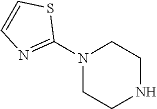 |
| 349 | 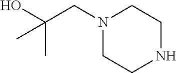 |

### Examples 10, 57 to 60, 143, 144, 351 and 367

Example compound 10 was synthesized through substantially the same synthesis method as a synthesis method of example compound 8 except for using (tert-butoxycarbonyl)-L-valine instead of (tert-butoxycarbonyl)-L-serine. In addition, example compounds 57 to 60, 143, 144, 351 and 367 were each prepared through substantially the same synthesis method as a synthesis method of example compound 10 except for using the compounds of the following table instead of 1-(2,4-difluorophenyl)piperazine as R₅—H.

TABLE 9

| Example | |
|---|---|
| No. | R₅—H |
| 57 | 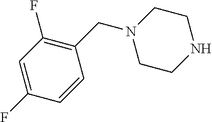 |
| 58 | 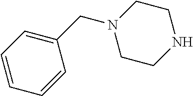 |
| 59 | 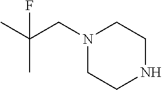 |
| 60 | 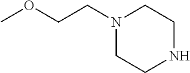 |
| 143 | 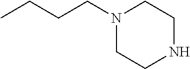 |
| 144 | 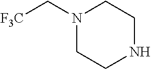 |
| 351 | 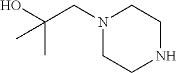 |
| 367 | 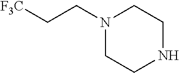 |

### Examples 162 to 167

Example compounds 162 to 167 were each prepared through substantially the same synthesis method as a synthesis method of example compound 9 except for using the compounds of the following table as N-protected amino acid instead of (tert-butoxycarbonyl)-L-alanine.

TABLE 10

| Example | |
|---|---|
| No. | Amino acid |
| 162 | 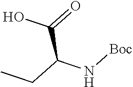 |
| 163 | 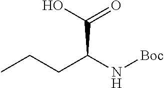 |
| 164 | 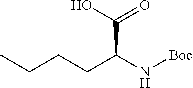 [SMILES: CCCC[C@H](NC(=O)OC(C)(C)C)C(=O)O] |
| 165 | 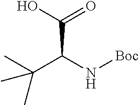 [SMILES: CC(C)(C)OC(=O)N[C@H](C(=O)O)C(C)(C)C] |
| 166 | 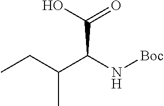 [SMILES: CCC(C)[C@H](NC(=O)OC(C)(C)C)C(=O)O] |
| 167 | 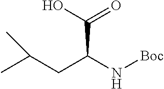 [SMILES: CC(C)C[C@H](NC(=O)OC(C)(C)C)C(=O)O] |

### Examples 172 to 175

Example compounds 172 to 175 were each prepared through substantially the same synthesis method as a synthesis method of example compound 143 except for using the compounds of the following table as N-protected amino acid instead of (tert-butoxycarbonyl)-L-valine.

TABLE 11

| Example | |
|---|---|
| No. | Amino acid |
| 172 | 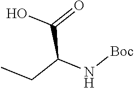 |
| 173 | 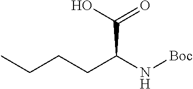 [SMILES: CCCC[C@H](NC(=O)OC(C)(C)C)C(=O)O] |
| 174 | 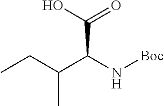 [SMILES: CCC(C)[C@H](NC(=O)OC(C)(C)C)C(=O)O] |
| 175 | 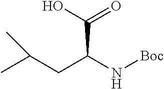 [SMILES: CC(C)C[C@H](NC(=O)OC(C)(C)C)C(=O)O] |

### Examples 211 to 215

Example compounds 211 to 215 were each prepared through substantially the same synthesis method as a synthesis method of example compound 144 except for using the compounds of the following table as N-protected amino acid instead of (tert-butoxycarbonyl)-L-valine.

TABLE 12

| Example | |
|---|---|
| No. | Amino acid |
| 211 | 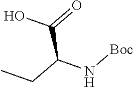 |
| 212 | 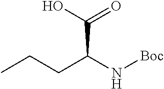 |
| 213 | 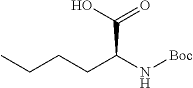 [SMILES: CCCC[C@H](NC(=O)OC(C)(C)C)C(=O)O] |
| 214 | 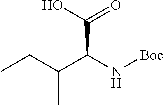 [SMILES: CCC(C)[C@H](NC(=O)OC(C)(C)C)C(=O)O] |
| 215 | 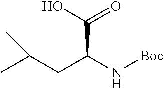 [SMILES: CC(C)C[C@H](NC(=O)OC(C)(C)C)C(=O)O] |

### Examples 248, 338 and 346

Example compound 338 was prepared through substantially the same synthesis method as a synthesis method of Example compound 240 except for using (S)-2-((tert-butoxycarbonyl)amino)butanoic acid instead of (tert-butoxycarbonyl)-L-alanine.

In addition, example compounds 248 and 346 were each prepared through substantially the same synthesis method as a synthesis method of example compound 338 except for using the compounds of the following table instead of 1-(2-fluoro-2-methylpropyl)piperazine as R₅—H.

TABLE 13

| Example | |
|---|---|
| No. | R₅—H |
| 248 | 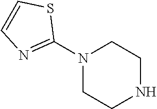 |
| 346 | 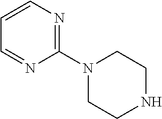 |

### Examples 11, 147, 148, 187 and 188

Example compound 11 was synthesized through substantially the same synthesis method as a synthesis method of example compound 12 except for using (S)-2-((tert-butoxycarbonyl)amino)-2-cyclohexylacetic acid instead of (S)-2-((tert-butoxycarbonyl)amino)-2-phenylacetic acid.

In addition, example compounds 147, 148, 187 and 188 were each prepared through substantially the same synthesis method as a synthesis method of example compound 11 except for using the compounds of the following table instead of 1-(2,4-difluorophenyl)piperazine as R₅—H.

TABLE 14

| Example | |
|---|---|
| No. | R₅—H |
| 147 | 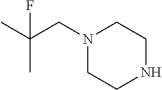 |
| 148 | 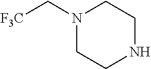 |
| 187 | 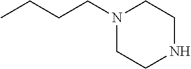 |
| 188 | 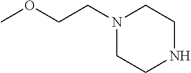 |

### Examples 168, 176, 216 and 339

Example compound 168 was synthesized through substantially the same synthesis method as a synthesis method of example compound 11 except for using (S)-2-((tert-butoxycarbonyl)amino)-2-cyclopropylacetic acid instead of (S)-2-((tert-butoxycarbonyl)amino)-2-cyclohexylacetic acid.

Example compounds 176, 216 and 339 were each prepared through substantially the same synthesis method as a synthesis method of example compound 168 except for using the compounds of the following table instead of 1-(2,4-difluorophenyl)piperazine as R₅—H.

TABLE 15

| Example | |
|---|---|
| No. | R₅—H |
| 176 | 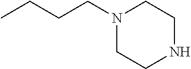 |
| 216 | 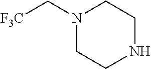 |
| 339 | 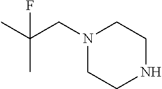 |

### Examples 169 and 177

Example compound 169 was synthesized through substantially the same synthesis method as a synthesis method of example compound 11 except for using (S)-2-((tert-butoxycarbonyl)amino)-2-cyclobutylacetic acid instead of (S)-2-((tert-butoxycarbonyl)amino)-2-cyclohexylacetic acid.

In addition, example compound 177 was prepared through substantially the same synthesis method as a synthesis method of example compound 169 except for using 1-butylpiperazine instead of 1-(2,4-difluorophenyl)piperazine.

### Examples 170, 178 and 217

Example compound 170 was synthesized through substantially the same synthesis method as a synthesis method of example compound 11 except for using (S)-2-((tert-butoxycarbonyl)amino)-2-cyclopentylacetic acid instead of (S)-2-((tert-butoxycarbonyl)amino)-2-cyclohexylacetic acid.

In addition, example compounds 178 and 217 were each synthesized through substantially the same synthesis method as a synthesis method of example compound 170 except for using the compounds of the following table instead of 1-(2,4-difluorophenyl)piperazine as R₅—H.

TABLE 16

| Example | |
|---|---|
| No. | R₅—H |
| 178 | 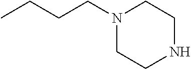 |
| 217 | 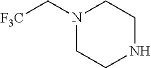 |

### Examples 40, 87, 89, 91, 189, 198, 199, 200, 201, 202, 203 and 300

Example compounds 40, 87, 89, 91, 189, 198, 199, 200, 201, 202, 203 and 300 were each synthesized through substantially the same synthesis method as a synthesis method of example compound 90 except for using the compounds of the following table instead of 1-(2-fluoro-2-methylpropyl)piperazine as R₅—H.

TABLE 17

| Example | |
|---|---|
| No. | R₅—H |
| 40 | 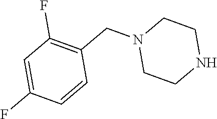 |
| 87 | 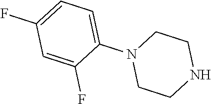 |
| 89 | 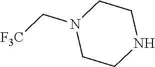 |
| 91 | 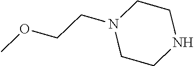 |
| 189 | 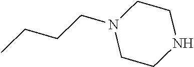 |
| 198 | 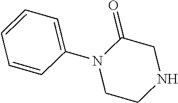 |
| 199 | 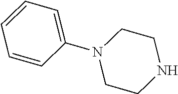 |
| 200 | 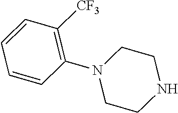 |
| 201 | 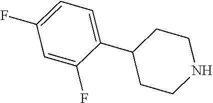 |
| 202 | 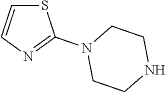 |
| 203 | 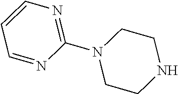 |
| 300 | 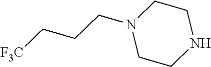 |

### Examples 207 to 210 and 218 to 222

Example compounds 207 to 210 and 218 to 222 were each synthesized through substantially the same synthesis method as a synthesis method of example compound 90 except for using the compounds of the following table instead of 1-(2-fluoro-2-methylpropyl)piperazine as R₅—H and using (S)-1-(tert-butoxycarbonyl)azetidin-2-carboxylic acid instead of (tert-butoxycarbonyl)-L-proline.

TABLE 18

| Example | |
|---|---|
| No. | R₅—H |
| 207 | 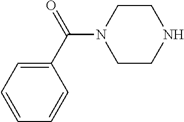 |
| 208 | 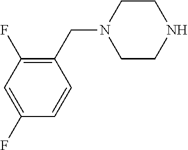 |
| 209 | 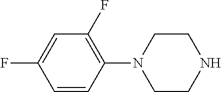 |
| 210 | 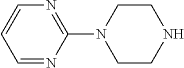 |
| 218 | 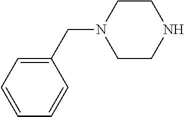 |
| 219 | 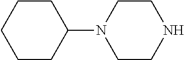 |
| 220 | 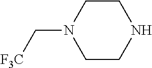 |
| 221 | 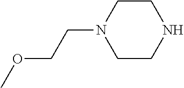 |
| 222 | 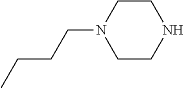 |

### Example 303: Synthesis of Compound 303, (S)-2-((7-amino-2-(furan-2-yl)-[1,2,4]triazolo[1,5-a][1,3,5]triazin-5-yl)amino)-1-(4-(2-fluoro-2-methylpropyl)piperazin-1-yl)-3-methoxypropan-1-one

### [Step 1] tert-butyl (S)-(1-(4-(2-fluoro-2-methylpropyl)piperazin-1-yl)-3-methoxy-1-oxopropan-2-yl)carbamate

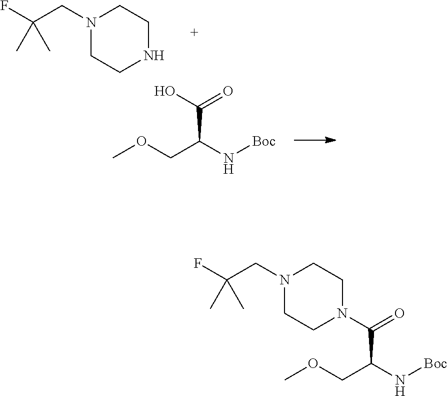

1-(2-Fluoro-2-methylpropyl)piperazine (0.500 g, 3.120 mmol), N-(tert-butoxycarbonyl)-O-methyl-L-serine (1.368 g, 6.241 mmol), 1-[bis(dimethylamino)methylene]-1H-1,2,3-triazolo[4,5-b]pyridinium 3-oxide hexafluorophosphate (HATU, 2.373 g, 6.241 mmol) and N,N-diisopropylethylamine (2.717 mL, 15.602 mmol) were dissolved in dichloromethane (15 mL) at room temperature, after which the resulting solution was stirred at the same temperature for 18 hours. Solvent was removed from the reaction mixture under reduced pressure, after which saturated aqueous solution of sodium hydrogen carbonate solution was poured into the resulting concentrate and an organic layer was extracted with ethyl acetate. The organic layer was washed with saturated aqueous solution of sodium hydrogen carbonate, dehydrated with anhydrous magnesium sulfate, filtered, and concentrated under reduced pressure. An obtained product was used without an additional purification process (title compound, 1.000 g, 88.7%, brown oil).

### [Step 2] (S)-2-amino-1-(4-(2-fluoro-2-methylpropyl)piperazin-1-yl)-3-methoxypropan-1-one

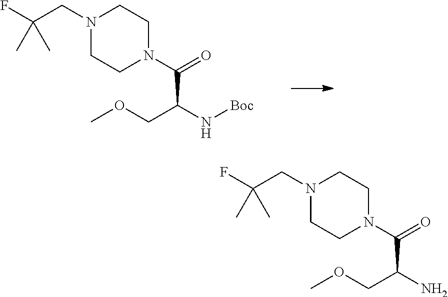 [SMILES: COC[C@H](NC(=O)OC(C)(C)C)C(=O)N1CCN(CC(C)(C)F)CC1 ; COC[C@H](N)C(=O)N1CCN(CC(C)(C)F)CC1]

Tert-butyl (S)-(1-(4-(2-fluoro-2-methylpropyl)piperazin-1-yl)-3-methoxy-1-oxopropan-2-yl)carbamate (0.200 g, 0.553 mmol) prepared in step 1 and hydrogen chloride (4.00 M solution in dioxane, 1.383 mL, 5.533 mmol) were dissolved in dichloromethane (10 mL) at room temperature, after which the resulting solution was stirred at the same temperature for 18 hours. Solvent was removed from the reaction mixture under reduced pressure, after which an obtained product was used without an additional purification process (title compound, 0.100 g, 69.2%, brown oil).

### [Step 3] (S)-2-((7-amino-2-(furan-2-yl)-[1,2,4]triazolo[1,5-a][1,3,5]triazin-5-yl)amino)-1-(4-(2-fluoro-2-methylpropyl)piperazin-1-yl)-3-methoxypropan-1-one

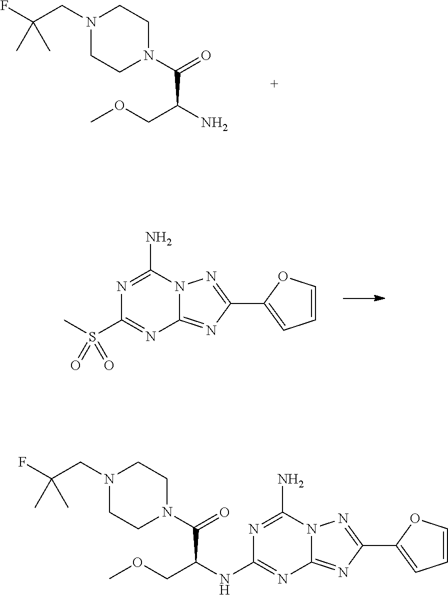 [SMILES: COC[C@H](N)C(=O)N1CCN(CC(C)(C)F)CC1 ; CS(=O)(=O)c1nc(N)n2nc(-c3ccco3)nc2n1 ; COC[C@H](Nc1nc(N)n2nc(-c3ccco3)nc2n1)C(=O)N1CCN(CC(C)(C)F)CC1]

(S)-2-Amino-1-(4-(2-fluoro-2-methylpropyl)piperazin-1-yl)-3-methoxypropan-1-one (0.100 g, 0.383 mmol) prepared in step 2, 2-(furan-2-yl)-5-(methylsulfonyl)-[1,2,4]triazolo[1,5-a][1,3,5]triazin-7-amine (0.054 g, 0.191 mmol) and sodium hydrogen carbonate (0.096 g, 1.148 mmol) were dissolved in acetonitrile (10 mL) at room temperature, after which the resulting solution was stirred at 70° C. for 18 hours to complete the reaction by lowering a temperature to room temperature. Solvent was removed from the reaction mixture under reduced pressure, after which the resulting concentrate was purified via column chromatography (SiO₂, 4 g cartridge; methanol/ethyl acetate=0 to 10%) and concentrated to obtain a title compound (0.015 g, 8.5%) as a white solid form.

¹H NMR (400 MHz, DMSO-d₆) δ 8.32 (s, 2H), 7.88 (s, 1H), 7.46 (dd, J=53.9, 8.2 Hz, 1H), 7.08 (d, J=3.2 Hz, 1H), 6.68 (dd, J=3.2, 1.7 Hz, 1H), 5.09 (dd, J=14.2, 6.3 Hz, 1H), 3.73-3.39 (m, 6H), 3.27 (s, 3H), 2.62-2.34 (m, 6H), 1.32 (d, J=21.5 Hz, 6H); LRMS (ES) m/z 462.5 (M⁺+1).

### Examples 304, 305, 306, 399 and 401

Example compounds 304, 305, 306, 399 and 401 were each prepared through substantially the same synthesis method as a synthesis method of example compound 303 except for using the compounds of the following table instead of 1-(2-fluoro-2-methylpropyl)piperazine as R₅—H of above reaction formula 1 in step 1.

TABLE 19

| Example | |
|---|---|
| No. | R₅—H |
| 304 | 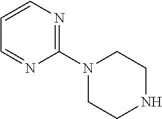 |
| 305 | 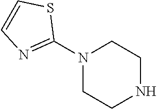 |
| 306 | 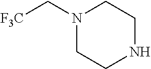 |
| 399 | 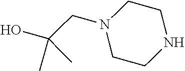 |
| 401 | 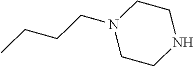 |

### Example 307: Synthesis of Compound 307

Example compound 307 was synthesized through substantially the same synthesis method as a synthesis method of example compound 304 except for using N-(tert-butoxycarbonyl)-O-ethyl-L-serine instead of N-(tert-butoxycarbonyl)-O-methyl-L-serine.

### Example 308: Synthesis of Compound 308

Example compound 308 was synthesized through substantially the same synthesis method as a synthesis method of example compound 306 except for using N-(tert-butoxycarbonyl)-O-ethyl-L-serine instead of N-(tert-butoxycarbonyl)-O-methyl-L-serine.

### Example 352: Synthesis of Compound 352

Example compound 352 was synthesized through substantially the same synthesis method as a preparation method of example compound 26 except for using 2-(furan-2-yl)-7-(methylsulfonyl)-[1,2,4]triazolo[1,5-c]pyrimidin-5-amine instead of 2-(furan-2-yl)-5-(methylsulfonyl)-[1,2,4]triazolo[1,5-a][1,3,5]triazin-7-amine of step 3.

### Examples 336 and 361: Synthesis of Compounds 336 and 361

Example compounds 336 and 361 were each synthesized through substantially the same synthesis method as a synthesis method of example compound 352 except for using 1-(2-methoxyethyl)piperazine and 1-butylpiperazine, respectively, instead of 1-(2,4-difluorophenyl)piperazine as R₅—H.

### Example 375: Synthesis of Compound 375

Example compound 375 was synthesized through substantially the same synthesis method as a synthesis method of example compound 119 except for using 2-(furan-2-yl)-7-(methylsulfonyl)-[1,2,4]triazolo[1,5-c]pyrimidin-5-amine instead of 2-(furan-2-yl)-5-(methylsulfonyl)-[1,2,4]triazolo[1,5-a][1,3,5]triazin-7-amine.

### Example 41: Synthesis of Compound 41

Example compound 41 was synthesized through substantially the same synthesis method as a synthesis method of example compound 16 except for using 2-(furan-2-yl)-5-(methylsulfonyl)-3a,7a-dihydrooxazolo[5,4-d]pyrimidin-7-amine instead of 2-(furan-2-yl)-5-(methylsulfonyl)-[1,2,4]triazolo[1,5-a][1,3,5]triazin-7-amine.

### Example 238: Synthesis of Compound 238

Example compound 238 was synthesized through substantially the same synthesis method as a synthesis method of example compound 48 except for using 2-(furan-2-yl)-5-(methylsulfonyl)-3a,7a-dihydrothiazolo[5,4-d]pyrimidin-7-amine instead of 2-(furan-2-yl)-5-(methylsulfonyl)-[1,2,4]triazolo[1,5-a][1,3,5]triazin-7-amine.

### Example 243: Synthesis of Compound 243

Example compound 243 was synthesized through substantially the same synthesis method as a synthesis method of example compound 189 except for using 2-(furan-2-yl)-5-(methylsulfonyl)-3a,7a-dihydrothiazolo[5,4-d]pyrimidin-7-amine instead of 2-(furan-2-yl)-5-(methylsulfonyl)-[1,2,4]triazolo[1,5-a][1,3,5]triazin-7-amine.

### Example 192: Synthesis of Compound 192

Example compound 192 was synthesized through substantially the same synthesis method as a synthesis method of example compound 48 except for using phenyl (piperazin-1-yl)methanone instead of 1-(2,2,2-trifluoroethyl) piperazine and using 5-(methylsulfonyl)-2-(thiazol-2-yl)-[1,2,4]triazolo[1,5-a][1,3,5]triazin-7-amine instead of 2-(furan-2-yl)-5-(methylsulfonyl)-[1,2,4]triazolo[1,5-a][1,3,5]triazin-7-amine.

### Example 193: Synthesis of Compound 193

Example compound 193 was synthesized through substantially the same synthesis method as a synthesis method of example compound 48 except for using 1-benzylpiperazine instead of 1-(2,2,2-trifluoroethyl)piperazine and using 5-(methylsulfonyl)-2-(thiazol-2-yl)-[1,2,4]triazolo[1,5-a][1,3,5]triazin-7-amine instead of 2-(furan-2-yl)-5-(methylsulfonyl)-[1,2,4]triazolo[1,5-a][1,3,5]triazin-7-amine.

### Example 194: Synthesis of Compound 194

Example compound 194 was synthesized through substantially the same synthesis method as a synthesis method of example compound 9 except for using 5-(methylsulfonyl)-2-(thiazol-2-yl)-[1,2,4]triazolo[1,5-a][1,3,5]triazin-7-amine instead of 2-(furan-2-yl)-5-(methylsulfonyl)-[1,2,4]triazolo[1,5-a][1,3,5]triazin-7-amine.

### Example 250: Synthesis of Compound 250

Example compound 250 was synthesized through substantially the same synthesis method as a synthesis method of example compound 148 except for using 5-(methylsulfonyl)-2-(thiazol-2-yl)-[1,2,4]triazolo[1,5-a][1,3,5]triazin-7-amine instead of 2-(furan-2-yl)-5-(methylsulfonyl)-[1,2,4]triazolo[1,5-a][1,3,5]triazin-7-amine.

### Example 67: Synthesis of Compound 67

Example compound 67 was synthesized through substantially the same synthesis method as a synthesis method of example compound 12 except for using 5-(methylsulfonyl)-2-(thiazol-2-yl)-[1,2,4]triazolo[1,5-a][1,3,5]triazin-7-amine instead of 2-(furan-2-yl)-5-(methylsulfonyl)-[1,2,4]triazolo[1,5-a][1,3,5]triazin-7-amine.

### Example 84: Synthesis of Compound 84

Example compound 84 was synthesized through substantially the same synthesis method as a synthesis method of example compound 139 except for using 2-(5-methylfuran-2-yl)-5-(methylsulfonyl)-[1,2,4]triazolo[1,5-a][1,3,5]triazine-7-amine instead of 2-(furan-2-yl)-5-(methylsulfonyl)-[1,2,4]triazolo[1,5-a][1,3,5]triazin-7-amine, and using 1-cyclohexylpiperazine instead of 1-cyclopropylpiperazine.

Analysis data of each of the compounds prepared as described above are shown in the table below.

TABLE 20

| Example | | |
|---|---|---|
| No. | Compound Name | Analysis Data |
| 1 | (S)-2-((7-amino-2-(furan-2-yl)- | ¹H NMR (400 MHz, Chloroform-d) δ 8.49 (dd, J = |
| | [1,2,4]triazolo[1,5-a][1,3,5]triazin-5- | 46.6, 9.3 Hz, 1H), 9.59 (d, J = 60.5 Hz, 1H), |
| | yl)amino)-1-(4-benzylpiperidin-1-yl)- | 7.50 (d, J = 6.2 Hz, 1H), 7.24-6.95 (m, 10H), |
| | 3-phenylpropan-1-one | 6.47 (d, J = 6.7 Hz, 1H), 6.13 (s, 1H), 5.65-5.51 |
| | | (m, 1H), 4.41 (t, J = 12.9 Hz, 1H), 4.21-3.92 (m, |
| | | 1H), 3.07-2.89 (m, 2H), 2.53-2.17 (m, 4H), |
| | | 1.69-1.29 (m, 4H), 0.72 (p, J = 14.1, 12.2 Hz, |
| | | 1H), −0.02 (q, J = 12.5 Hz, 1H); LRMS (ES) m/z |
| | | 523.1 (M⁺ + 1). |
| 3 | (S)-2-((7-amino-2-(furan-2-yl)- | ¹H NMR (400 MHz, Chloroform-d) δ 7.41 (d, J = |
| | [1,2,4]triazolo[1,5-a][1,3,5]triazin-5- | 8.9 Hz, 1H), 7.24-6.87 (m, 7H), 9.53 (d, J = |
| | yl)amino)-1-(4- | 76.8 Hz, 1H), 8.71-8.16 (m, 1H), 6.87-6.46 |
| | (phenoxymethyl)piperidin-1-yl)-3- | (m, 3H), 6.46-6.26 (m, 1H), 6.09 (s, 1H), 5.51 |
| | phenylpropan-1-one | (q, J = 8.4 Hz, 1H), 4.53-4.29 (m, 1H), 4.29- |
| | | 4.01 (m, 1H), 3.69-3.50 (m, 1H), 3.50-3.29 |
| | | (m, 1H), 3.11-2.84 (m, 3H), 2.45-2.24 (m, |
| | | 1H), 1.94-1.30 (m, 4H), 0.81-0.60 (m, 1H), |
| | | 0.08-−0.18 (m, 1H); LRMS (ES) m/z 539.1 |
| | | (M⁺ + 1). |
| 7 | (S)-2-((7-amino-2-(furan-2-yl)- | ¹H NMR (400 MHz, DMSO-d₆) δ 8.50-8.07 (m, |
| | [1,2,4]triazolo[1,5-a][1,3,5]triazin-5- | 2H), 7.88 (dd, J = 1.8, 0.8 Hz, 1H), 7.68 (dd, J = |
| | yl)amino)-1-(4-(2,4- | 24.2, 7.7 Hz, 1H), 7.53-7.28 (m, 6H), 7.20 (ddd, |
| | difluorophenyl)piperazin-1-yl)-2- | J = 12.0, 8.9, 2.7 Hz, 1H), 7.10-6.92 (m, 3H), |
| | phenylethan-1-one | 6.69 (dd, J = 3.4, 1.8 Hz, 1H), 6.10-5.97 (m, |
| | | 1H), 3.84-3.42 (m, 5H), 3.06-2.76 (m, 3H); |
| | | LRMS (ES) m/z 532.4 (M⁺ + 1). |
| 8 | (S)-2-((7-amino-2-(furan-2-yl)- | ¹H NMR (400 MHz, DMSO-d₆) δ 8.51-8.07 (m, |
| | [1,2,4]triazolo[1,5-a][1,3,5]triazin-5- | 2H), 7.88 (s, 1H), 7.43-7.26 (m, 1H), 7.26- |
| | yl)amino)-1-(4-(2,4- | 6.95 (m, 4H), 6.69 (d, J = 3.5 Hz, 1H), 5.08- |
| | difluorophenyl)piperazin-1-yl)-3- | 4.90 (m, 2H), 3.96-3.50 (m, 6H), 3.22-2.84 |
| | hydroxypropan-1-one | (m, 4H); LRMS (ES) m/z 486.4 (M⁺ + 1). |
| 9 | (S)-2-((7-amino-2-(furan-2-yl)- | ¹H NMR (400 MHz, DMSO-d₆) δ 8.48-8.06 |
| | [1,2,4]triazolo[1,5-a][1,3,5]triazin-5- | (m, 2H), 7.88 (s, 1H), 7.64-7.45 (m, 1H), 7.28- |
| | yl)amino)-1-(4-(2,4- | 6.93 (m, 4H), 6.68 (dt, J = 3.7, 1.7 Hz, 1H), 5.01- |
| | difluorophenyl)piperazin-1- | 4.83 (m, 1H), 3.87-3.46 (m, 4H), 3.24-2.84 |
| | yl)propan-1-one | (m, 4H), 1.35-1.24 (m, 3H); LRMS (ES) m/z |
| | | 470.4 (M⁺ + 1). |
| 10 | (S)-2-((7-amino-2-(furan-2-yl)- | ¹H NMR (400 MHz, DMSO-d₆) δ 8.45-8.05 (m, |
| | [1,2,4]triazolo[1,5-a][1,3,5]triazin-5- | 1H), 7.93-7.81 (m, 1H), 7.65-7.40 (m, 1H), |
| | yl)amino)-1-(4-(2,4- | 7.28-6.91 (m, 4H), 6.71-6.64 (m, 1H), 4.80- |
| | difluorophenyl)piperazin-1-yl)-3- | 4.58 (m, 1H), 4.04-3.43 (m, 4H), 3.29-2.77 |
| | methylbutan-1-one | (m, 4H), 2.18-1.97 (m, 1H), 1.04-0.87 (m, |
| | | 6H); LRMS (ES) m/z 498.4 (M⁺ + 1). |
| 11 | (S)-2-((7-amino-2-(furan-2-yl)- | ¹H NMR (400 MHz, DMSO-d₆) δ 8.41-8.05 (m, |
| | [1,2,4]triazolo[1,5-a][1,3,5]triazin-5- | 1H), 7.93-7.83 (m, 1H), 7.61-7.40 (m, 1H), |
| | yl)amino)-2-cyclohexyl-1-(4-(2,4- | 7.29-7.13 (m, 2H), 7.12-6.93 (m, 2H), 6.69 (d, |
| | difluorophenyl)piperazin-1-yl)ethan- | J = 3.8 Hz, 1H), 4.82-4.66 (m, 1H), 4.04-3.44 |
| | 1-one | (m, 4H), 3.27-2.75 (m, 4H), 1.90-1.54 (m, |
| | | 5H), 1.28-0.95 (m, 6H); LRMS (ES) m/z 538.5 |
| | | (M⁺ + 1). |
| 12 | (S)-(1-(7-amino-2-(furan-2-yl)- | ¹H NMR (400 MHz, DMSO-d₆) δ 8.61-8.08 (m, |
| | [1,2,4]triazolo[1,5-a][1,3,5]triazin-5- | 2H), 7.88 (s, 1H), 7.30-6.97 (m, 5H), 6.68 (t, J = |
| | yl)pyrrolidin-2-yl)(4-(2,4- | 3.4 Hz, 1H), 5.11-4.98 (m, 1H), 3.94-3.47 |
| | difluorophenyl)piperazin-1- | (m, 6H), 3.20-2.84 (m, 4H), 2.37-2.20 (m, |
| | yl)methanone | 1H), 2.05-1.81 (m, 4H); LRMS (ES) m/z 496.4 |
| | | (M⁺ + 1). |
| 13 | (S)-2-((7-amino-2-(furan-2-yl)- | ¹H NMR (400 MHz, DMSO-d₆) δ 8.49-8.02 |
| | [1,2,4]triazolo[1,5-a][1,3,5]triazin-5- | (m, 2H), 7.87 (s, 1H), 7.78-7.58 (m, 1H), 7.48- |
| | yl)amino)-1-(4-(2,4- | 7.33 (m, 1H), 7.32-7.13 (m, 6H), 7.11-6.98 (m, |
| | difluorobenzyl)piperazin-1-yl)-3- | 2H), 6.71-6.62 (m, 1H), 5.09-4.96 (m, 1H), |
| | phenylpropan-1-one | 3.64-2.88 (m, 10H), 2.39-1.92 (m, 4H); |
| | | LRMS (ES) m/z 560.5 (M⁺ + 1). |
| 14 | (S)-2-((7-amino-2-(furan-2-yl)- | ¹H NMR (400 MHz, DMSO-d₆) δ 8.55-8.14 (m, |
| | [1,2,4]triazolo[1,5-a][1,3,5]triazin-5- | 2H), 7.88 (s, 1H), 7.58 (dd, J = 30.5, 7.6 Hz, 1H), |
| | yl)amino)-1-(4-(2,4- | 7.50-7.26 (m, 6H), 7.19 (t, J = 9.8 Hz, 1H), 7.10- |
| | difluorobenzyl)piperazin-1-yl)-2- | 6.99 (m, 2H), 6.68 (d, J = 3.5 Hz, 1H), 6.02- |
| | phenylethan-1-one | 5.93 (m, 1H), 3.70-3.31 (m, 6H), 2.42-1.76 |
| | | (m, 4H); LRMS (ES) m/z 546.4 (M⁺ + 1). |
| 15 | 2-((7-amino-2-(furan-2-yl)- | ¹H NMR (400 MHz, DMSO-d₆) δ 8.54-8.08 |
| | [1,2,4]triazolo[1,5-a][1,3,5]triazin-5- | (m, 2H), 7.88 (s, 1H), 7.53-6.97 (m, 5H), 6.68 |
| | yl)amino)-1-(4-(2,4- | (d, J = 3.2 Hz, 1H), 4.21-4.04 (m, 2H), 3.58- |
| | difluorobenzyl)piperazin-1-yl)ethan- | 3.41 (m, 6H), 2.50-2.27 (m, 5H); LRMS (ES) |
| | 1-one | m/z 470.4 (M⁺ + 1). |
| 16 | (S)-(1-(7-amino-2-(furan-2-yl)- | ¹H NMR (400 MHz, DMSO-d₆) δ 8.61-8.10 (m, |
| | [1,2,4]triazolo[1,5-a][1,3,5]triazin-5- | 2H), 7.87 (s, 1H), 7.54-7.02 (m, 4H), 6.68 (dd, |
| | yl)pyrrolidin-2-yl)(4-(2,4- | J = 3.3, 1.7 Hz, 1H), 5.06-4.90 (m, 1H), 3.72- |
| | difluorobenzyl)piperazin-1- | 3.17 (m, 8H), 2.75-2.60 (m, 1H), 2.47-2.18 |
| | yl)methanone | (m, 4H), 2.04-1.78 (m, 3H); LRMS (ES) m/z |
| | | 510.5 (M⁺ + 1). |
| 19 | (S)-2-((7-amino-2-(furan-2-yl)- | ¹H NMR (400 MHz, Chloroform-d) δ 9.80-9.37 |
| | [1,2,4]triazolo[1,5-a][1,3,5]triazin-5- | (m, 1H), 8.78-8.51 (m, 1H), 7.61 (ddd, J = 4.3, |
| | yl)amino)-1-(4-morpholinopiperidin- | 1.8, 0.8 Hz, 1H), 7.32-7.10 (m, 6H), 6.58 (s, |
| | 1-yl)-3-phenylpropan-1-one | 1H), 6.32 (s, 1H), 5.73-5.58 (m, 1H), 4.61-4.48 |
| | | (m, 1H), 4.26 (dd, J = 34.8, 13.8 Hz, 1H), 3.75- |
| | | 3.68 (m, 4H), 3.17-3.02 (m, 2H), 2.63-2.50 |
| | | (m, 3H), 2.45-2.38 (m, 3H), 1.71-1.63 (m, 1H), |
| | | 1.33-1.25 (m, 3H), 1.14-1.01 (m, 1H); LRMS |
| | | (ES) m/z 518.5 (M⁺ + 1). |
| 20 | (S)-2-((7-amino-2-(furan-2-yl)- | ¹H NMR (400 MHz, Chloroform-d) δ 7.61 (s, |
| | [1,2,4]triazolo[1,5-a][1,3,5]triazin-5- | 1H), 3.99-3.85 (m, 1H), 3.85-3.66 (m, 1H), |
| | yl)amino)-1-(4-benzyl-1,4-diazepan-1- | 3.66-3.49 (m, 3H), 3.43 (dd, J = 15.4, 6.6 Hz, |
| | yl)-3-phenylpropan-1-one | 1H), 3.18-2.93 (m, 2H), 2.93-2.49 (m, 2H), |
| | | 9.82 (s, 1H), 8.67 (d, J = 9.4 Hz, 1H), 7.42-7.01 |
| | | (m, 9H), 6.65-6.48 (m, 1H), 6.30 (s, 1H), 5.76- |
| | | 5.47 (m, 1H), 2.49-2.06 (m, 2H); LRMS (ES) |
| | | m/z 538.5 (M⁺ + 1). |
| 27 | (S)-(1-(7-amino-2-(furan-2-yl)- | ¹H NMR (400 MHz, Chloroform-d) δ 7.59-7.52 |
| | [1,2,4]triazolo[1,5-a][1,3,5]triazin-5- | (m, 1H), 7.43-7.31 (m, 4H), 7.31-7.25 (m, 1H), |
| | yl)pyrrolidin-2-yl)(4-benzylpiperazin- | 7.24-7.14 (m, 1H), 6.58-6.51 (m, 1H), 6.30 (s, |
| | 1-yl)methanone | 1H), 6.04 (s, 1H), 4.98 (ddd, J = 84.6, 8.4, 3.2 |
| | | Hz, 1H), 3.95-3.53 (m, 7H), 2.63-2.52 (m, |
| | | 1H), 2.49-2.34 (m, 2H), 2.32-2.22 (m, 1H), |
| | | 2.15 (ddt, J = 11.1, 7.3, 4.2 Hz, 1H), 2.08-2.00 |
| | | (m, 2H), 1.27 (s, 1H); LRMS (ES) m/z 474.4 |
| | | (M⁺ + 1). |
| 28 | (S)-2-((7-amino-2-(furan-2-yl)- | ¹H NMR (400 MHz, Chloroform-d) δ 9.73-9.43 |
| | [1,2,4]triazolo[1,5-a][1,3,5]triazin-5- | (m, 1H), 8.59 (d, J = 9.3 Hz, 1H), 7.61 (s, 1H), |
| | yl)amino)-1-(4-(2-fluoro-2- | 7.30-7.12 (m, 6H), 6.58 (dd, J = 3.4, 1.8 Hz, |
| | methylpropyl)piperazin-1-yl)-3- | 1H), 6.29 (s, 1H), 5.66 (d, J = 8.2 Hz, 1H), 3.81- |
| | phenylpropan-1-one | 3.62 (m, 2H), 3.62-3.38 (m, 2H), 3.14-3.01 |
| | | (m, 2H), 2.62-2.47 (m, 2H), 2.40-2.29 (m, |
| | | 2H), 2.29-2.18 (m, 1H), 1.90 (s, 1H), 1.42-1.29 |
| | | (m, 6H); LRMS (ES) m/z 508.5 (M⁺ + 1). |
| 29 | (S)-(1-(7-amino-2-(furan-2-yl)- | ¹H NMR (400 MHz, DMSO-d₆) δ 8.55-8.22 (m, |
| | [1,2,4]triazolo[1,5-a][1,3,5]triazin-5- | 3H), 7.88 (dd, J = 1.8, 0.8 Hz, 1H), 7.52-7.01 |
| | yl)piperidin-2-yl)(4-(2,4- | (m, 3H), 6.69 (dd, J = 3.4, 1.8 Hz, 1H), 5.65- |
| | difluorobenzyl)piperazin-1- | 5.49 (m, 1H), 4.60-4.43 (m, 1H), 3.61-3.27 |
| | yl)methanone | (m, 7H), 2.47-2.22 (m, 4H), 1.90-1.30 (m, |
| | | 6H); LRMS (ES) m/z 524.4 (M⁺ + 1). |
| 55 | (S)-(1-(7-amino-2-(furan-2-yl)- | ¹H NMR (400 MHz, DMSO-d₆) δ 8.63-8.06 (m, |
| | [1,2,4]triazolo[1,5-a][1,3,5]triazin-5- | 2H), 7.90-7.84 (m, 1H), 7.09-6.99 (m, 1H), |
| | yl)pyrrolidin-2-yl)(4,4- | 6.72-6.63 (m, 1H), 5.12-4.97 (m, 1H), 3.93- |
| | difluoropiperidin-1-yl)methanone | 3.76 (m, 1H), 3.76-3.56 (m, 3H), 3.55-3.40 |
| | | (m, 1H), 3.03 (d, J = 23.3 Hz, 1H), 2.44-2.17 |
| | | (m, 2H), 2.07-1.80 (m, 6H) ; LRMS (ES) m/z |
| | | 419.3 (M⁺ + 1). |
| 37 | (2S,4R)-1-(7-amino-2-(furan-2-yl)- | ¹H NMR (400 MHz, DMSO-d₆) δ 8.60-8.11 (m, |
| | [1,2,4]triazolo[1,5-a][1,3,5]triazin-5- | 2H), 7.88 (s, 1H), 7.29-6.98 (m, 4H), 6.68 (q, J = |
| | yl)-4-hydroxypyrrolidin-2-yl)(4-(2,4- | 2.5, 1.7 Hz, 1H), 5.15-4.98 (m, 2H), 4.45- |
| | difluorophenyl)piperazin-1- | 4.33 (m, 1H), 3.95-3.37 (m, 6H), 3.27-2.80 |
| | yl)methanone | (m, 4H), 2.30-2.18 (m, 1H), 2.11-1.92 (m, 1H); |
| | | LRMS (ES) m/z 512.4 (M⁺ + 1). |
| 40 | (S)-(1-(7-amino-2-(5-methylfuran-2- | ¹H NMR (400 MHz, Chloroform-d) δ 7.45-7.30 |
| | yl)-[1,2,4]triazolo[1,5-a][1,3,5]triazin- | (m, 1H), 7.10 (dd, J = 19.0, 3.3 Hz, 1H), 6.94- |
| | 5-yl)pyrrolidin-2-yl)(4-(2,4- | 6.77 (m, 2H), 6.19-6.03 (m, 2H), 6.00-5.75 |
| | difluorobenzyl)piperazin-1- | (m, 1H), 5.17-4.87 (m, 1H), 3.95-3.81 (m, 1H), |
| | yl)methanone | 3.80-3.69 (m, 2H), 3.69-3.53 (m, 4H), 2.93- |
| | | 2.79 (m, 1H), 2.68-2.53 (m, 1H), 2.50-2.36 |
| | | (m, 5H), 2.27 (tt, J = 9.0, 5.0 Hz, 1H), 2.22- |
| | | 2.08 (m, 1H), 2.06-1.93 (m, 2H), 1.90-1.86 |
| | | (m, 1H); LRMS (ES) m/z 524.1 (M⁺ + 1). |
| 41 | (S)-(1-(7-amino-2-(furan-2- | ¹H NMR (400 MHz, Chloroform-d) δ 7.61 (s, |
| | yl)oxazolo[5,4-d]pyrimidin-5- | 1H), 7.52-7.39 (m, 1H), 7.29 (s, 2H), 7.08 (s, |
| | yl)pyrrolidin-2-yl)(4-(2,4- | 1H), 6.87 (dt, J = 26.2, 8.9 Hz, 2H), 6.59 (s, 1H), |
| | difluorobenzyl)piperazin-1- | 5.35-5.30 (m, 1H), 5.25-5.08 (m, 1H), 5.00- |
| | yl)methanone | 4.95 (m, 1H), 3.95-3.49 (m, 9H), 2.71-2.39 |
| | | (m, 4H), 2.32-2.09 (m, 2H), 2.05-1.98 (m, |
| | | 2H); LRMS (ES) m/z 510.1 (M⁺ + 1). |
| 57 | (S)-2-((7-amino-2-(furan-2-yl)- | ¹H NMR (400 MHz, Chloroform-d) δ 9.68- |
| | [1,2,4]triazolo[1,5-a][1,3,5]triazin-5- | 9.29 (m, 1H), 7.63-7.58 (m, 1H), 7.48-7.33 |
| | yl)amino)-1-(4-(2,4- | (m, 1H), 8.46 (d, J = 9.4 Hz, 1H), 7.19 (dd, J = |
| | difluorobenzyl)piperazin-1-yl)-3- | 3.4, 0.8 Hz, 1H), 6.90-6.74 (m, 2H), 6.58 (s, |
| | methylbutan-1-one | 1H), 6.37-6.08 (m, 1H), 5.12-4.94 (m, 1H), |
| | | 4.25-4.00 (m, 1H), 3.87 (s, 2H), 3.62 (s, 3H), |
| | | 2.52-2.39 (m, 2H), 2.14-2.00 (m, 1H), 2.00- |
| | | 1.61 (m, 2H), 0.97 (dd, J = 16.0, 6.6 Hz, 6H); |
| | | LRMS (ES) m/z 512.5 (M⁺ + 1). |
| 58 | (S)-2-((7-amino-2-(furan-2-yl)- | ¹H NMR (400 MHz, Chloroform-d) δ 9.51 (s, |
| | [1,2,4]triazolo[1,5-a][1,3,5]triazin-5- | 1H), 8.46 (d, J = 9.4 Hz, 1H), 8.37-7.66 (m, |
| | yl)amino)-1-(4-benzylpiperazin-1-yl)- | OH), 7.60 (s, 1H), 7.53 (s, 0H), 7.41-7.13 (m, |
| | 3-methylbutan-1-one | 5H), 6.57 (dd, J = 3.4, 1.8 Hz, 1H), 6.23 (s, 1H), |
| | | 5.16-4.91 (m, 1H), 4.21-4.05 (m, 1H), 3.97- |
| | | 3.66 (m, 2H), 3.66-3.37 (m, 3H), 2.53-2.29 |
| | | (m, 2H), 2.16-1.96 (m, 1H), 1.96-1.65 (m, 2H), |
| | | 1.10-0.76 (m, 6H); LRMS (ES) m/z 476.5 (M⁺+ |
| | | 1). |
| 59 | (S)-2-((7-amino-2-(furan-2-yl)- | ¹H NMR (400 MHz, Chloroform-d) δ 9.59 (s, |
| | [1,2,4]triazolo[1,5-a][1,3,5]triazin-5- | 1H), 8.49 (d, J = 9.5 Hz, 1H), 7.61 (s, 1H), 7.20 |
| | yl)amino)-1-(4-(2-fluoro-2- | (s, 1H), 6.58 (s, 1H), 6.20 (s, 1H), 5.09 (t, J = 9.5 |
| | methylpropyl)piperazin-1-yl)-3- | Hz, 1H), 4.09-3.84 (m, 2H), 3.78-3.58 (m, |
| | methylbutan-1-one | 2H), 2.81-2.72 (m, 1H), 2.62-2.53 (m, 2H), |
| | | 2.53-2.41 (m, 2H), 2.13-2.09 (m, 1H), 1.42 (d, |
| | | J = 4.9 Hz, 3H), 1.37 (d, J = 4.9 Hz, 3H), 1.02- |
| | | 0.93 (m, 6H); LRMS (ES) m/z 460.5 (M⁺ + 1). |
| 60 | (S)-2-((7-amino-2-(furan-2-yl)- | ¹H NMR (400 MHz, Chloroform-d) δ 9.54 (s, |
| | [1,2,4]triazolo[1,5-a][1,3,5]triazin-5- | 1H), 8.47 (d, J = 9.5 Hz, 1H), 7.61 (s, 1H), 7.19 (s, |
| | yl)amino)-1-(4-(2- | 1H), 6.58 (s, 1H), 6.21 (s, 1H), 5.06 (t, J = 9.4 Hz, |
| | methoxyethyl)piperazin-1-yl)-3- | 1H), 4.21-4.13 (m, 1H), 3.93-3.85 (m, 2H), |
| | methylbutan-1-one | 3.71-3.48 (m, 3H), 3.38 (s, 3H), 2.66 (d, J = |
| | | 14.0 Hz, 4H), 2.50 (s, 2H), 2.11-2.07 (m, 1H), |
| | | 1.03-0.92 (m, 6H); LRMS (ES) m/z 444.5 (M⁺+ |
| | | 1). |
| 65 | (S)-2-(4-(1-(7-amino-2-(furan-2-yl)- | ¹H NMR (400 MHz, DMSO-d₆) δ 8.74-8.07 (m, |
| | [1,2,4]triazolo[1,5-a][1,3,5]triazin-5- | 2H), 7.94-7.83 (m, 1H), 7.17-6.91 (m, 1H), |
| | yl)azetidine-2-carbonyl)piperazin-1- | 6.80-6.61 (m, 1H), 5.31-5.16 (m, 1H), 4.15- |
| | yl)-1-morpholinoethan-1-one | 3.87 (m, 2H), 3.67-3.37 (m, 12H), 3.23 (s, 2H), |
| | | 2.72-2.54 (m, 2H), 2.46-2.30 (m, 3H), 2.17- |
| | | 2.06 (m, 1H) ; LRMS (ES) m/z 497.6 (M⁺ + 1). |
| 67 | (S)-(1-(7-amino-2-(thiazol-2-yl)- | ¹H NMR (400 MHz, Chloroform-d) δ 7.87-7.82 |
| | [1,2,4]triazolo[1,5-a][1,3,5]triazin-5- | (m, 1H), 7.39-7.33 (m, 1H), 7.03-6.75 (m, 1H), |
| | yl)pyrrolidin-2-yl)(4-(2,4- | 6.75-6.63 (m, 2H), 6.39-6.14 (m, 2H), 4.91 |
| | difluorophenyl)piperazin-1- | (ddd, J = 64.5, 8.3, 3.1 Hz, 1H), 3.95-3.44 (m, |
| | yl)methanone | 6H), 3.43-3.31 (m, 1H), 2.99-2.75 (m, 3H), |
| | | 2.24-1.97 (m, 2H), 1.97-1.79 (m, 2H); LRMS |
| | | (ES) m/z 513.5 (M⁺ + 1). |
| 78 | 1-((7-amino-2-(furan-2-yl)- | ¹H NMR (400 MHz, Chloroform-d) δ 8.67 (s, |
| | [1,2,4]triazolo[1,5-a][1,3,5]triazin-5- | 1H), 7.62-7.52 (m, 3H), 7.38-7.30 (m, 2H), |
| | yl)-L-prolyl)-N-phenylpiperidin-4- | 7.18-7.12 (m, 2H), 6.54 (dd, J = 3.4, 1.7 Hz, 1H), |
| | carboxamide | 6.25 (d, J = 30.5 Hz, 1H), 4.88 (dd, J = 8.5, 4.1 |
| | | Hz, 1H), 4.65 (d, J = 12.8 Hz, 1H), 4.01 (d, J = |
| | | 14.4 Hz, 1H), 3.92-3.69 (m, 2H), 3.15 (t, J = |
| | | 13.5 Hz, 1H), 2.81 (dq, J = 11.2, 5.5, 4.5 Hz, 1H), |
| | | 2.64-2.53 (m, 2H), 2.31 (dq, J = 14.4, 9.4, 8.1 |
| | | Hz, 1H), 2.20-2.07 (m, 1H), 2.05-1.91 (m, |
| | | 5H); LRMS (ES) m/z 502.6 (M⁺ + 1). |
| 79 | 1-((7-amino-2-(furan-2-yl)- | ¹H NMR (400 MHz, Chloroform-d) δ 8.77 (s, |
| | [1,2,4]triazolo[1,5-a][1,3,5]triazin-5- | 1H), 8.37-8.28 (m, 1H), 8.14 (d, J = 8.4 Hz, 1H), |
| | yl)-L-prolyl)-N-(pyridin-2- | 7.78 (ddd, J = 8.4, 7.4, 1.9 Hz, 1H), 7.57 (dd, J = |
| | yl)piperidin-4-carboxamide | 1.8, 0.8 Hz, 1H), 7.21 (dd, J = 3.4, 0.9 Hz, 1H), |
| | | 7.17-7.07 (m, 1H), 6.55 (dd, J = 3.4, 1.8 Hz, 1H), |
| | | 4.89 (dd, J = 8.5, 4.0 Hz, 1H), 4.70 (d, J = 13.0 |
| | | Hz, 1H), 4.05 (d, J = 14.5 Hz, 1H), 3.99-3.82 |
| | | (m, 2H), 3.17 (t, J = 13.5 Hz, 1H), 2.74-2.46 (m, |
| | | 3H), 2.32 (dq, J = 14.3, 9.4, 8.1 Hz, 1H), 2.17 (dq, |
| | | J = 13.8, 7.7 Hz, 1H), 2.09-1.88 (m, 5H); LRMS |
| | | (ES) m/z 503.5 (M⁺ + 1). |
| 80 | 1-((7-amino-2-(furan-2-yl)- | ¹H NMR (400 MHz, Chloroform-d) δ 9.51 (d, J = |
| | [1,2,4]triazolo[1,5-a][1,3,5]triazin-5- | 21.7 Hz, 1H), 8.79 (s, 1H), 8.45 (d, J = 2.6 Hz, |
| | yl)-L-prolyl)-N-(pyrazin-2- | 1H), 8.31 (dd, J = 2.6, 1.5 Hz, 1H), 7.57 (dd, J = |
| | yl)piperidin-4-carboxamide | 1.8, 0.8 Hz, 1H), 7.22 (dd, J = 3.5, 0.9 Hz, 1H), |
| | | 6.56 (dd, J = 3.5, 1.7 Hz, 1H), 4.89 (dd, J = 8.6, |
| | | 3.9 Hz, 1H), 4.70 (d, J = 13.0 Hz, 1H), 4.06 (d, J = |
| | | 14.4 Hz, 1H), 3.98-3.81 (m, 2H), 3.19 (t, J = |
| | | 13.5 Hz, 1H), 2.79 (s, 0H), 2.59 (dtt, J = 29.1, |
| | | 12.5, 5.9 Hz, 2H), 2.40-2.28 (m, 1H), 2.23- |
| | | 2.10 (m, 1H), 2.09-1.92 (m, 5H); LRMS (ES) |
| | | m/z 504.6 (M⁺ + 1). |
| 81 | 1-((7-amino-2-(furan-2-yl)- | ¹H NMR (400 MHz, Chloroform-d) δ 8.64 (s, |
| | [1,2,4]triazolo[1,5-a][1,3,5]triazin-5- | 1H), 7.58-7.53 (m, 1H), 7.39-7.23 (m, 5H), |
| | yl)-L-prolyl)-N-benzylpiperidin-4- | 7.16 (d, J = 3.4 Hz, 1H), 6.57-6.44 (m, 2H), 6.13 |
| | carboxamide | (s, 1H), 4.87 (dd, J = 8.6, 3.9 Hz, 1H), 4.70-4.60 |
| | | (m, 1H), 4.53-4.38 (m, 2H), 4.01 (d, J = 14.2 |
| | | Hz, 1H), 3.87 (ddq, J = 17.9, 11.6, 6.6, 5.8 Hz, |
| | | 2H), 3.16 (t, J = 12.7 Hz, 1H), 2.64-2.52 (m, |
| | | 3H), 2.38-2.24 (m, 1H), 2.14 (ddt, J = 17.6, 11.2, |
| | | 6.2 Hz, 1H), 2.07-1.70 (m, 5H); LRMS (ES) m/z |
| | | 516.5 (M⁺ + 1). |
| 84 | (S)-(1-(7-amino-2-(5-methylfuran-2- | ¹H NMR (400 MHz, DMSO-d₆) δ 8.63-7.94 (m, |
| | yl)-[1,2,4]triazolo[1,5-a][1,3,5]triazin- | 2H), 6.98-6.82 (m, 1H), 6.36-6.20 (m, 1H), |
| | 5-yl)pyrrolidin-2-yl)(4- | 5.06-4.88 (m, 1H), 3.78-3-43 (m, 6H), 3.27- |
| | cyclohexylpiperazin-1-yl)methanone | 3.08 (m, 1H), 2.87-2.62 (m, 1H), 2.44-2.13 |
| | | (m, 6H), 1.96-1.88 (m, 2H), 1.88-1.66 (m, |
| | | 5H), 1.59 (d, J = 12.3 Hz, 1H), 1.36-1.00 (m, |
| | | 6H) ; LRMS (ES) m/z 480.6 (M⁺ + 1). |
| 87 | (S)-(1-(7-amino-2-(5-methylfuran-2- | ¹H NMR (400 MHz, DMSO-d₆) δ 8.51-8.05 (m, |
| | yl)-[1,2,4]triazolo[1,5-a][1,3,5]triazin- | 2H), 7.28-6.87 (m, 4H), 6.29 (tt, J = 2.4, 1.2 |
| | 5-yl)pyrrolidin-2-yl)(4-(2,4- | Hz, 1H), 5.10-4.97 (m, 1H), 3.94-3.41 (m, |
| | difluorophenyl)piperazin-1- | 6H), 3.23-2.86 (m, 4H), 2.40-2.18 (m, 4H), |
| | yl)methanone | 2.02-1.80 (m, 3H); LRMS (ES) m/z 510.5 (M⁺+ |
| | | 1). |
| 89 | (S)-(1-(7-amino-2-(5-methylfuran-2- | ¹H NMR (400 MHz, DMSO-d₆) δ 8.55-8.07 (m, |
| | yl)-[1,2,4]triazolo[1,5-a][1,3,5]triazin- | 2H), 6.94 (dd, J = 6.2, 3.2 Hz, 1H), 6.29 (ddd, J = |
| | 5-yl)pyrrolidin-2-yl)(4-(2,2,2- | 3.2, 2.0, 1.1 Hz, 1H), 5.02-4.91 (m, 1H), 3.78- |
| | trifluoroethyl)piperazin-1- | 3.16 (m, 8H), 3.02-2.47 (m, 4H), 2.36 (d, J = |
| | yl)methanone | 3.0 Hz, 3H), 2.31-2.19 (m, 1H), 2.06-1.77 (m, |
| | | 3H); LRMS (ES) m/z 480.1 (M⁺ + 1). |
| 91 | (S)-(1-(7-amino-2-(5-methylfuran-2- | ¹H NMR (400 MHz, DMSO-d₆) δ 8.56-8.06 (m, |
| | yl)-[1,2,4]triazolo[1,5-a][1,3,5]triazin- | 2H), 6.94 (t, J = 3.5 Hz, 1H), 6.29 (tt, J = 2.5, 1.2 |
| | 5-yl)pyrrolidin-2-yl)(4-(2- | Hz, 1H), 5.04-4.91 (m, 1H), 3.78-3.15 (m, |
| | methoxyethyl)piperazin-1- | 15H), 2.80-2.16 (m, 6H), 2.00-1.77 (m, 3H); |
| | yl)methanone | LRMS (ES) m/z 456.6 (M⁺ + 1). |
| 92 | (R)-(1-(7-amino-2-(furan-2-yl)- | ¹H NMR (400 MHz, Chloroform-d) δ 7.60-7.53 |
| | [1,2,4]triazolo[1,5-a][1,3,5]triazin-5- | (m, 1H), 7.25-7.13 (m, 1H), 6.55 (ddd, J = 7.4, |
| | yl)pyrrolidin-2-yl)(4-(2- | 3.4, 1.8 Hz, 1H), 6.10 (d, J = 28.3 Hz, 2H), 5.13- |
| | methoxyethyl)piperazin-1- | 4.82 (m, 1H), 3.97-3.49 (m, 8H), 3.39 (d, J = |
| | yl)methanone | 3.0 Hz, 3H), 2.89-2.61 (m, 4H), 2.59-2.41 (m, |
| | | 2H), 2.36-2.10 (m, 2H), 2.09-1.94 (m, 3H), |
| | | 1.32-1.20 (m, 1H); LRMS (ES) m/z 442.5 (M⁺ + |
| | | 1). |
| 93 | (R)-(1-(7-amino-2-(furan-2-yl)- | ¹H NMR (400 MHz, Chloroform-d) δ 7.56 (ddd, |
| | [1,2,4]triazolo[1,5-a][1,3,5]triazin-5- | J = 10.9, 1.8, 0.8 Hz, 1H), 7.19 (ddd, J = 21.7, 3.4, |
| | yl)pyrrolidin-2-yl)(4-(2-fluoro-2- | 0.8 Hz, 1H), 6.55 (ddd, J = 10.1, 3.4, 1.8 Hz, 1H), |
| | methylpropyl)piperazin-1- | 6.17 (s, 2H), 5.14-4.83 (m, 1H), 3.96-3.41 (m, |
| | yl)methanone | 6H), 2.93 (s, 1H), 2.71 (s, 1H), 2.65-2.37 (m, |
| | | 4H), 2.35-2.23 (m, 1H), 2.21-2.11 (m, 1H), |
| | | 2.09-1.95 (m, 2H), 1.47-1.41 (m, 3H), 1.41- |
| | | 1.35 (m, 3H); LRMS (ES) m/z 458.4 (M⁺ + 1). |
| 94 | (R)-(1-(7-amino-2-(furan-2-yl)- | ¹H NMR (400 MHz, Chloroform-d) δ 7.61-7.53 |
| | [1,2,4]triazolo[1,5-a][1,3,5]triazin-5- | (m, 1H), 7.19 (dd, J = 26.6, 3.4 Hz, 1H), 6.59- |
| | yl)pyrrolidin-2-yl)(4-(2,2,2- | 6.52 (m, 1H), 6.18 (s, 2H), 5.00 (ddd, J = 66.5, |
| | trifluoroethyl)piperazin-1- | 8.4, 3.1 Hz, 1H), 3.96-3.53 (m, 8H), 3.23-3.01 |
| | yl)methanone | (m, 2H), 2.81-2.65 (m, 2H), 2.37-2.09 (m, |
| | | 2H), 2.09-1.94 (m, 2H); LRMS (ES) m/z 466.3 |
| | | (M⁺ + 1). |
| 95 | (R)-(1-(7-amino-2-(furan-2-yl)- | ¹H NMR (400 MHz, Chloroform-d) δ 7.57 (dd, J = |
| | [1,2,4]triazolo[1,5-a][1,3,5]triazin-5- | 7.0, 1.7 Hz, 1H), 7.34 (qd, J = 9.1, 3.6 Hz, 5H), |
| | yl)pyrrolidin-2-yl)(4-benzylpiperazin- | 7.24-7.14 (m, 1H), 6.59-6.52 (m, 1H), 6.18- |
| | 1-yl)methanone | 5.62 (m, 2H), 5.13-4.84 (m, 1H), 3.97-3.70 |
| | | (m, 4H), 3.70-3.49 (m, 4H), 2.90-2.55 (m, |
| | | 2H), 2.54-2.32 (m, 2H), 2.32-2.10 (m, 2H), |
| | | 2.09-1.93 (m, 2H); LRMS (ES) m/z 474.4 (M⁺ + |
| | | 1). |
| 96 | (R)-(1-(7-amino-2-(furan-2-yl)- | ¹H NMR (400 MHz, Chloroform-d) δ 7.57 (ddd, |
| | [1,2,4]triazolo[1,5-a][1,3,5]triazin-5- | J = 8.4, 1.8, 0.8 Hz, 1H), 7.44-7.32 (m, 1H), |
| | yl)pyrrolidin-2-yl)(4-(2,4- | 7.24-7.14 (m, 1H), 6.94-6.76 (m, 2H), 6.59- |
| | difluorobenzyl)piperazin-1- | 6.51 (m, 1H), 6.16 (s, 2H), 5.13-4.85 (m, 1H), |
| | yl)methanone | 3.96-3.42 (m, 8H), 2.88-2.35 (m, 4H), 2.34- |
| | | 2.08 (m, 2H), 2.05-1.95 (m, 2H); LRMS (ES) |
| | | m/z 510.3 (M⁺ + 1). |
| 97 | (R)-(1-(7-amino-2-(furan-2-yl)- | ¹H NMR (400 MHz, Chloroform-d) δ 7.60-7.53 |
| | [1,2,4]triazolo[1,5-a][1,3,5]triazin-5- | (m, 1H), 7.25-7.13 (m, 1H), 7.13-7.03 (m, 1H), |
| | yl)pyrrolidin-2-yl)(4-(2,4- | 6.98-6.78 (m, 2H), 6.59-6.51 (m, 1H), 6.22 (s, |
| | difluorophenyl)piperazin-1- | 2H), 5.06 (ddd, J = 71.4, 8.3, 3.0 Hz, 1H), 4.07- |
| | yl)methanone | 3.62 (m, 6H), 3.21-2.91 (m, 4H), 2.40-2.13 |
| | | (m, 2H), 2.13-1.95 (m, 2H); LRMS (ES) m/z |
| | | 496.4 (M⁺ + 1). |
| 98 | 4-((7-amino-2-(furan-2-yl)- | ¹H NMR (400 MHz, Chloroform-d) δ 7.56 (s, |
| | [1,2,4]triazolo[1,5-a][1,3,5]triazin-5- | 1H), 7.52-7.38 (m, 5H), 7.24-7.15 (m, 1H), |
| | yl)-D-prolyl)piperazin-1- | 6.58-6.51 (m, 1H), 6.25 (d, J = 16.7 Hz, 2H), |
| | yl)(phenyl) methanone | 5.10-4.88 (m, 1H), 4.10-3.35 (m, 10H), 2.39- |
| | | 1.95 (m, 4H); LRMS (ES) m/z 488.6 (M⁺ + 1). |
| 107 | (S)-(1-(7-amino-2-(furan-2-yl)- | ¹H NMR (400 MHz, DMSO-d₆) δ 8.77-8.04 (m, |
| | [1,2,4]triazolo[1,5-a][1,3,5]triazin-5- | 2H), 7.93-7.82 (m, 1H), 7.11-6.99 (m, 1H), |
| | yl)azetidin-2-yl)(4-(2- | 6.73-6.61 (m, 1H), 5.30-5.13 (m, 1H), 4.11- |
| | methoxyethyl)piperazin-1- | 3.89 (m, 2H), 3.78-3.36 (m, 7H), 3.24 (s, 4H), |
| | yl) methanone | 2.77-2.56 (m, 2H), 2.47-2.18 (m, 3H), 2.10 (s, |
| | | 1H); LRMS (ES) m/z 428.6 (M⁺ + 1). |
| 108 | (S)-(1-(7-amino-2-(furan-2-yl)- | ¹H NMR (400 MHz, DMSO-d₆) δ 8.82-8.02 |
| | [1,2,4]triazolo[1,5-a][1,3,5]triazin-5- | (m, 2H), 7.93-7.82 (m, 1H), 7.05 (s, 1H), 6.74- |
| | yl)azetidin-2-yl)(4-(2-fluoro-2- | 6.61 (m, 1H), 5.29-5.15 (m, 1H), 4.06-3.90 |
| | methylpropyl)piperazin-1- | (m, 2H), 3.75-3.36 (m, 5H), 2.75-2.55 (m, |
| | yl)methanone | 3H), 2.49-2.21 (m, 3H), 2.11 (s, 1H), 1.36 (s, |
| | | 3H), 1.31 (s, 3H); LRMS (ES) m/z 444.6 (M⁺ + 1). |
| 109 | (S)-(1-(7-amino-2-(furan-2-yl)- | ¹H NMR (400 MHz, DMSO-d₆) δ 8.79-8.02 (m, |
| | [1,2,4]triazolo[1,5-a][1,3,5]triazin-5- | 2H), 7.92-7.82 (m, 1H), 7.55-7.34 (m, 5H), |
| | yl)azetidin-2-yl)(4-benzoylpiperazin- | 7.12-6.98 (m, 1H), 6.74-6.61 (m, 1H), 5.41- |
| | 1-yl)methanone | 5.06 (m, 1H), 4.09-3.92 (m, 2H), 3.90-3.43 |
| | | (m, 8H), 2.64 (s, 1H), 2.19 (s, 1H); LRMS (ES) |
| | | m/z 474.6 (M⁺ + 1). |
| 110 | (S)-4-(1-(7-amino-2-(furan-2-yl)- | ¹H NMR (400 MHz, DMSO-d₆) δ 8.84-8.02 |
| | [1,2,4]triazolo[1,5-a][1,3,5]triazine-5- | (m, 3H), 7.93-7.83 (m, 1H), 7.53-7.41 (m, |
| | yl)azetidin-2-carbonyl)-N- | 2H), 7.31-7.19 (m, 2H), 7.11-7.00 (m, 1H), |
| | phenylpiperazin-1-carboxamide | 6.99-6.88 (m, 1H), 6.67 (s, 1H), 5.29 (dd, J = |
| | | 9.1, 5.3 Hz, 1H), 4.14-3.91 (m, 2H), 3.76-3.42 |
| | | (m, 8H), 2.75-2.56 (m, 1H), 2.20 (s, 1H); LRMS |
| | | (ES) m/z 489.6 (M⁺ + 1). |
| 112 | (S)-(1-(7-amino-2-(furan-2-yl)- | ¹H NMR (400 MHz, DMSO-d₆) δ 8.77-8.04 (m, |
| | [1,2,4]triazolo[1,5-a][1,3,5]triazin-5- | 2H), 7.91-7.84 (m, 1H), 7.29-7.20 (m, 1H), |
| | yl)azetidin-2-yl)(4-(2,4- | 7.21-7.09 (m, 1H), 7.09-6.97 (m, 2H), 6.72- |
| | difluorophenyl)piperazin-1- | 6.64 (m, 1H), 5.36-5.21 (m, 1H), 4.08-3.91 |
| | yl)methanone | (m, 2H), 3.89-3-42 (m, 5H), 3.11-2.83 (m, |
| | | 3H), 2.76-2.57 (m, 1H), 2.27-2.10 (m, 1H); |
| | | LRMS (ES) m/z 482.6 (M⁺ + 1). |
| 113 | (S)-(1-(7-amino-2-(furan-2-yl)- | ¹H NMR (400 MHz, DMSO-d₆) δ 8.73-8.07 (m, |
| | [1,2,4]triazolo[1,5-a][1,3,5]triazin-5- | 2H), 7.94-7.81 (m, 1H), 7.48-7.38 (m, 1H), |
| | yl)pyrrolidin-2-yl)(3-benzylazetidin- | 7.34-7.25 (m, 2H), 7.25-7.15 (m, 2H), 7.11- |
| | 1-yl)methanone | 6.95 (m, 1H), 6.73-6.63 (m, 1H), 4.61-4.34 |
| | | (m, 2H), 4.26-3.76 (m, 2H), 3.73-3-47 (m, |
| | | 3H), 3.16-2.82 (m, 3H), 2.28-2.08 (m, 1H), |
| | | 2.08-1.78 (m, 3H); LRMS (ES) m/z 445.6 (M⁺ + |
| | | 1). |
| 114 | (S)-(1-(7-amino-2-(furan-2-yl)- | ¹H NMR (400 MHz, DMSO-d₆) δ 8.64-8.11 (m, |
| | [1,2,4]triazolo[1,5-a][1,3,5]triazin-5- | 2H), 7.94-7.85 (m, 1H), 7.35-7.23 (m, 2H), |
| | yl)pyrrolidin-2-yl)(3- | 7.23-7.15 (m, 1H), 7.11-7.03 (m, 1H), 7.02- |
| | (phenoxymethyl)azetidin-1- | 6.88 (m, 2H), 6.73-6.65 (m, 1H), 4.71-4.47 |
| | yl)methanone | (m, 2H), 4.47-4.21 (m, 2H), 4.16 (t, J = 6.9 Hz, |
| | | 1H), 4.09-3.92 (m, 2H), 3.76-3.55 (m, 3H), |
| | | 3.20-2.98 (m, 1H), 2.28-2.12 (m, 1H), 2.11- |
| | | 1.82 (m, 2H); LRMS (ES) m/z 461.6 (M⁺ + 1). |
| 118 | (S)-2-((7-amino-2-(furan-2-yl)- | ¹H NMR (400 MHz, Chloroform-d) δ 8.94 (s, |
| | [1,2,4]triazolo[1,5-a][1,3,5]triazin-5- | 1H), 7.59 (s, 1H), 8.20 (d, J = 8.9 Hz, 1H), 7.20 |
| | yl)amino)-1-(4-(2- | (dd, J = 3.4, 0.8 Hz, 1H), 6.57 (d, J = 3.4, 1.8 Hz, |
| | methoxyethyl)piperazin-1-yl)propan- | 1H), 6.33 (s, 1H), 5.40 (dq, J = 8.7, 6.9 Hz, 1H), |
| | 1-one | 4.02 (dt, J = 13.1, 4.6 Hz, 1H), 3.85 (d, J = 14.6 |
| | | Hz, 1H), 3.73 (dt, J = 12.8, 5.3 Hz, 1H), 3.63- |
| | | 3.51 (m, 3H), 3.37 (s, 3H), 2.72-2.51 (m, 5H), |
| | | 2.46 (ddd, J = 11.2, 7.7, 3.2 Hz, 1H), 1.42 (d, J = |
| | | 7.0 Hz, 3H); LRMS (ES) m/z 416.5 (M⁺ + 1). |
| 119 | (S)-2-((7-amino-2-(furan-2-yl)- | ¹H NMR (400 MHz, Chloroform-d) δ 8.87 (s, |
| | [1,2,4]triazolo[1,5-a][1,3,5]triazin-5- | 1H), 8.18 (d, J = 8.9 Hz, 1H), 7.85 (s, 0H), 7.60 |
| | yl)amino)-1-(4-(2-fluoro-2- | (dd, J = 1.9, 0.8 Hz, 1H), 7.34 (s, 0H), 7.20 (dd, |
| | methylpropyl)piperazin-1-yl)propan- | J = 3.4, 0.8 Hz, 1H), 6.57 (dd, J = 3.4, 1.8 Hz, |
| | 1-one | 1H), 6.28 (s, 1H), 5.50-5.31 (m, 1H), 4.04- |
| | | 3.82 (m, 1H), 3.82-3.51 (m, 3H), 3.35 (s, 0H), |
| | | 3.01-2.26 (m, 6H), 1.49-1.39 (m, 6H), 1.39- |
| | | 1.30 (m, 3H); LRMS (ES) m/z 432.6 (M⁺ + 1). |
| 120 | (S)-(1-(7-amino-2-(furan-2-yl)- | ¹H NMR (400 MHz, DMSO-d₆) δ 8.61-8.11 (m, |
| | [1,2,4]triazolo[1,5-a][1,3,5]triazin-5- | 2H), 7.88 (ddd, J = 2.6, 1.8, 0.8 Hz, 1H), 7.23- |
| | yl)pyrrolidin-2-yl)(4-(2- | 6.95 (m, 5H), 6.68 (ddd, J = 4.2, 3.4, 1.8 Hz, 1H), |
| | fluorophenyl)piperazin-1- | 5.05 (ddd, J = 13.9, 8.8, 2.9 Hz, 1H), 3.94-3.53 |
| | yl)methanone | (m, 6H), 3.27-2.88 (m, 4H), 2.38-2.20 (m, |
| | | 1H), 1.97-1.81 (m, 3H); LRMS (ES) m/z 478.6 |
| | | (M⁺ + 1). |
| 121 | (S)-(1-(7-amino-2-(furan-2-yl)- | ¹H NMR (400 MHz, DMSO-d₆) δ 8.67-8.10 (m, |
| | [1,2,4]triazolo[1,5-a][1,3,5]triazin-5- | 2H), 7.87 (ddd, J = 5.3, 1.8, 0.8 Hz, 1H), 7.17- |
| | yl)pyrrolidin-2-yl)(4-(4- | 6.93 (m, 5H), 6.67 (ddd, J = 7.9, 3.4, 1.8 Hz, 1H), |
| | fluorophenyl)piperazin-1- | 5.11-4.98 (m, 1H), 3.88-3-37 (m, 7H), 3.12 (m, |
| | yl)methanone | 3H), 2.38-2.17 (m, 1H), 1.99-1.82 (m, 3H); |
| | | LRMS (ES) m/z 478.6 (M⁺ + 1). |
| 122 | (S)-(1-(7-amino-2-(furan-2-yl)- | ¹H NMR (400 MHz, DMSO-d₆) δ 8.31 (m, 2H), |
| | [1,2,4]triazolo[1,5-a][1,3,5]triazin-5- | 7.87 (m, 1H), 7.11-6.86 (m, 5H), 6.68 (td, J = |
| | yl)pyrrolidin-2-yl)(4-(2- | 3.2, 1.8 Hz, 1H), 5.06 (m, 1H), 3.88-3.45 (m, |
| | methoxyphenyl)piperazin-1- | 9H), 3.24-2.79 (m, 4H), 2.30 (m, 3H), 1.94 (m, |
| | yl)methanone | 3H); LRMS (ES) m/z 490.6 (M⁺ + 1). |
| 123 | Ethyl 4-(4-((7-amino-2-(furan-2-yl)- | ¹H NMR (400 MHz, DMSO-d₆) δ 8.31 (m, 2H), |
| | [1,2,4]triazolo[1,5-a][1,3,5]triazin-5- | 7.91-7.75 (m, 3H), 7.08-6.92 (m, 3H), 6.66 |
| | yl)-L-prolyl)piperazin-1-yl)benzoate | (ddd, J = 20.1, 3.4, 1.8 Hz, 1H), 5.10-4.98 (m, |
| | | 1H), 4.26 (m, 2H), 3.93-3.76 (m, 2H), 3.75- |
| | | 3.40 (m, 8H), 2.29 (m, 1H), 1.94 (m, 3H), 1.31 |
| | | (td, J = 7.1, 2.0 Hz, 3H); LRMS (ES) m/z 532.6 |
| | | (M⁺ + 1). |
| 124 | (S)-(1-(7-amino-2-(furan-2-yl)- | ¹H NMR (400 MHz, DMSO-d₆) δ 8.59 (ddd, J = |
| | [1,2,4]triazolo[1,5-a][1,3,5]triazin-5- | 11.4, 4.9, 1.7 Hz, 1H), 8.54-8.06 (m, 3H), 7.87 |
| | yl)pyrrolidin-2-yl)(4-(3- | (ddd, J = 5.6, 1.9, 0.8 Hz, 1H), 7.27 (td, J = 7.9, |
| | (trifluoromethyl)pyridin-2- | 4.8 Hz, 1H), 7.06 (ddd, J = 8.9, 3.4, 0.8 Hz, 1H), |
| | yl)piperazin-1-yl)methanone | 6.68 (ddd, J = 6.3, 3-4, 1.8 Hz, 1H), 5.10-4.98 |
| | | (m, 1H), 3.91-3.37 (m, 7H), 3.28-3.08 (m, |
| | | 3H), 2.29 (m, 1H), 1.93 (m, 3H); LRMS (ES) m/z |
| | | 529.6 (M⁺ + 1). |
| 125 | 2-(4-((7-amino-2-(furan-2-yl)- | ¹H NMR (400 MHz, DMSO-d₆) δ 8.31 (d, J = |
| | [1,2,4]triazolo[1,5-a][1,3,5]triazin-5- | 116.8 Hz, 2H), 7.94-7.80 (m, 1H), 7.21 (dd, J = |
| | yl)-L-prolyl)piperazin-1-yl)thiazole | 6.0, 3.6 Hz, 1H), 7.11-6.95 (m, 1H), 6.90 (dd, J = |
| | | 8.9, 3.6 Hz, 1H), 6.74-6.60 (m, 1H), 5.10- |
| | | 4.97 (m, 1H), 3.92-3.39 (m, 10H), 2.35-2.21 |
| | | (m, 1H), 1.93 (q, J = 7.3 Hz, 3H); LRMS (ES) m/z |
| | | 467.5 (M⁺ + 1). |
| 126 | (S)-(1-(7-amino-2-(furan-2-yl)- | ¹H NMR (400 MHz, DMSO-d₆) δ 8.54-8.05 (m, |
| | [1,2,4]triazolo[1,5-a][1,3,5]triazin-5- | 4H), 7.91-7.81 (m, 2H), 7.02 (ddd, J = 39.0, |
| | yl)pyrrolidin-2-yl)(4-(pyrazin-2- | 3.4, 0.9 Hz, 1H), 6.67 (ddd, J = 13.5, 3.4, 1.8 Hz, |
| | yl)piperazin-1-yl)methanone | 1H), 5.11-4.97 (m, 1H), 3.96-3.48 (m, 10H), |
| | | 2.30 (m, 1H), 2.01-1.81 (m, 3H); LRMS (ES) |
| | | m/z 462.6 (M⁺ + 1). |
| 127 | (S)-(1-(7-amino-2-(furan-2-yl)- | ¹H NMR (400 MHz, DMSO-d₆) δ 8.41 (dd, J = |
| | [1,2,4]triazolo[1,5-a][1,3,5]triazin-5- | 4.8, 2.8 Hz, 4H), 7.86 (ddd, J = 9.1, 1.8, 0.8 Hz, |
| | yl)pyrrolidin-2-yl)(4-(pyrimidin-2- | 1H), 7.04 (ddd, J = 22.3, 3.4, 0.8 Hz, 1H), 6.74- |
| | yl)piperazin-1-yl)methanone | 6.62 (m, 2H), 5.10-4.99 (m, 1H), 4.09-3.43 |
| | | (m, 10H), 2.32 (m, 1H), 1.94 (m, 3H); LRMS |
| | | (ES) m/z 462.6 (M⁺ + 1). |
| 128 | (S)-(1-(7-amino-2-(furan-2-yl)- | ¹H NMR (400 MHz, DMSO-d₆) δ 8.29 (m, 2H), |
| | [1,2,4]triazolo[1,5-a][1,3,5]triazin-5- | 7.96-7.83 (m, 1H), 7.57-7.17 (m, 5H), 7.07 (m, |
| | yl)pyrrolidin-2-yl)(4-phenylpiperidin- | 1H), 6.71 (m, 1H), 5.07 (m, 1H), 4.49 (m, 1H), |
| | 1-yl)methanone | 4.32-4.06 (m, 1H), 3.76-3.58 (m, 2H), 3.20 |
| | | (m, 1H), 2.91-2.59 (m, 2H), 2.35-1.76 (m, 7H), |
| | | 1.71-1.39 (m, 2H); LRMS (ES) m/z 459.6 (M⁺ + |
| | | 1). |
| 134 | (S)-(1-(7-amino-2-(furan-2-yl)- | ¹H NMR (400 MHz, DMSO-d₆) δ 8.64-8.02 |
| | [1,2,4]triazolo[1,5-a][1,3,5]triazin-5- | (m, 2H), 7.90-7.82 (m, 1H), 7.10-7.00 (m, |
| | yl)pyrrolidin-2-yl)(4- | 1H), 6.73-6.62 (m, 1H), 5.00 (td, J = 8.8, 3.2 |
| | methylpiperazin-1-yl)methanone | Hz, 1H), 3.85-3.49 (m, 7H), 2.90-2.61 (m, |
| | | 1H), 2.43-2.16 (m, 6H), 2.00-1.77 (m, 3H); |
| | | LRMS (ES) m/z 398.7 (M⁺ + 1). |
| 135 | (S)-(1-(7-amino-2-(furan-2-yl)- | ¹H NMR (400 MHz, DMSO-d₆) δ 8.62-7.95 (m, |
| | [1,2,4]triazolo[1,5-a][1,3,5]triazin-5- | 2H), 7.90-7.79 (m, 1H), 7.11-6.98 (m, 1H), |
| | yl)pyrrolidin-2-yl)(4-propylpiperazin- | 6.74-6.59 (m, 1H), 5.06-4.89 (m, 1H), 3.83- |
| | 1-yl)methanone | 3.47 (m, 5H), 3.30-3.16 (m, 1H), 2.76-2.56 |
| | | (m, 1H), 2.48-2.38 (m, 1H), 2.41-2.06 (m, |
| | | 5H), 1.97-1.76 (m, 3H), 1.59-1.38 (m, 2H), |
| | | 0.96-0.85 (m, 3H); LRMS (ES) m/z 426.6 |
| | | (M⁺ + 1). |
| 136 | (S)-(1-(7-amino-2-(furan-2-yl)- | ¹H NMR (400 MHz, DMSO-d₆) δ 8.71-8.09 (m, |
| | [1,2,4]triazolo[1,5-a][1,3,5]triazin-5- | 2H), 7.87 (dd, J = 1.8, 0.9 Hz, 1H), 7.08-6.95 |
| | yl)pyrrolidin-2-yl)(4- | (m, 1H), 6.70-6.63 (m, 1H), 5.09-4.89 (m, |
| | isopropylpiperazin-1-yl)methanone | 1H), 4.42-3.73 (m, 3H), 3.73-3.52 (m, 4H), |
| | | 3.16-2.64 (m, 4H), 2.34-2.15 (m, 1H), 2.11- |
| | | 1.78 (m, 3H), 1.48-0.97 (m, 6H); LRMS (ES) |
| | | m/z 426.5 (M⁺ + 1). |
| 137 | (S)-(1-(7-amino-2-(furan-2-yl)- | ¹H NMR (400 MHz, DMSO-d₆) δ 8.61-8.01 (m, |
| | [1,2,4]triazolo[1,5-a][1,3,5]triazin-5- | 2H), 7.89-7.82 (m, 1H), 7.08-6.98 (m, 1H), |
| | yl)pyrrolidin-2-yl)(4-(tert- | 6.70-6.62 (m, 1H), 4.99 (td, J = 8.4, 3.1 Hz, |
| | butyl)piperazin-1-yl)methanone | 1H), 3.80-3.08 (m, 7H), 2.86-2.59 (m, 2H), |
| | | 2.43-2.18 (m, 2H), 2.00-1.75 (m, 3H), 1.05 (d, |
| | | J = 7.5 Hz, 9H); LRMS (ES) m/z 440.6 (M⁺ + 1). |
| 138 | (S)-(1-(7-amino-2-(furan-2-yl)- | ¹H NMR (400 MHz, DMSO-d₆) δ 8.69-8.00 |
| | [1,2,4]triazolo[1,5-a][1,3,5]triazin-5- | (m, 2H), 7.93-7.82 (m, 1H), 7.11-7.00 (m, 1H), |
| | yl)pyrrolidin-2-yl)(4-pentylpiperazin- | 6.73-6.62 (m, 1H), 5.07-4.89 (m, 1H), 3.73- |
| | 1-yl)methanone | 3.48 (m, 5H), 3.33-3.18 (m, 1H), 2.73-2.55 |
| | | (m, 1H), 2.48-2.39 (m, 1H), 2.37-2.10 (m, |
| | | 5H), 1.98-1.76 (m, 3H), 1.52-1.36 (m, 2H), |
| | | 1.36-1.18 (m, 4H), 0.94-0.80 (m, 3H); LRMS |
| | | (ES) m/z 454.7 (M⁺ + 1). |
| 139 | (S)-(1-(7-amino-2-(furan-2-yl)- | ¹H NMR (400 MHz, DMSO-d₆) δ 8.73-7.99 (m, |
| | [1,2,4]triazolo[1,5-a][1,3,5]triazin-5- | 2H), 7.93-7.82 (m, 1H), 7.11-7.01 (m, 1H), |
| | yl)pyrrolidin-2-yl)(4- | 6.73-6.61 (m, 1H), 5.10-4.90 (m, 1H), 3.79- |
| | cyclopropylpiperazin-1-yl)methanone | 3.45 (m, 5H), 3.31-3.14 (m, 2H), 2.89-2.71 |
| | | (m, 1H), 2.69-2.56 (m, 1H), 2.45-2.35 (m, |
| | | 1H), 2.35-2.19 (m, 1H), 2.00-1.77 (m, 3H), |
| | | 1.78-1.61 (m, 1H), 0.54-0.40 (m, 2H), 0.40- |
| | | 0.27 (m, 2H); LRMS (ES) m/z 424.7 (M⁺ + 1). |
| 140 | (S)-(1-(7-amino-2-(furan-2-yl)- | ¹H NMR (400 MHz, DMSO-d₆) δ 8.64-7.98 (m, |
| | [1,2,4]triazolo[1,5-a][1,3,5]triazin-5- | 2H), 7.90-7.83 (m, 1H), 7.06 (t, J = 3.3 Hz, 1H), |
| | yl)pyrrolidin-2-yl)(4-(2- | 6.72-6.61 (m, 1H), 5.06-4.92 (m, 1H), 3.74- |
| | isopropoxyethyl)piperazin-1- | 3.42 (m, 8H), 3.32-3.19 (m, 1H), 2.78-2.66 |
| | yl)methanone | (m, 1H), 2.59-2.51 (m, 3H), 2.44-2.19 (m, |
| | | 3H), 2.00-1.76 (m, 3H), 1.09 (d, J = 6.0 Hz, |
| | | 6H); LRMS (ES) m/z 470.7 (M⁺ + 1). |
| 141 | (S)-(1-(7-amino-2-(furan-2-yl)- | ¹H NMR (400 MHz, DMSO-d₆) δ 8.77-7.99 (m, |
| | [1,2,4]triazolo[1,5-a][1,3,5]triazin-5- | 2H), 7.89-7.82 (m, 1H), 7.09-7.01 (m, 1H), |
| | yl)pyrrolidin-2-yl)(4-(2-(2- | 6.71-6.63 (m, 1H), 5.09-4.91 (m, 1H), 3.71- |
| | methoxyethoxy)ethyl)piperazin-1- | 3.47 (m, 9H), 3.47-3.41 (m, 2H), 3.25 (s, 3H), |
| | yl)methanone | 2.79-2.65 (m, 1H), 2.59-2.52 (m, 4H), 2.43- |
| | | 2.19 (m, 3H), 1.97-1.77 (m, 3H); LRMS (ES) |
| | | m/z 486.6 (M⁺ + 1). |
| 142 | (1-((7-amino-2-(furan-2-yl)- | ¹H NMR (400 MHz, DMSO-d₆) δ 8.20 (m, 2H), |
| | [1,2,4]triazolo[1,5-a][1,3,5]triazin-5- | 7.91-7.84 (m, 1H), 7.14-7.01 (m, 1H), 6.68 (m, |
| | yl)-L-prolyl)piperidin-4-yl)(piperidin- | 1H), 5.11-4.88 (m, 1H), 4.40-3.92 (m, 2H), |
| | 1-yl)methanone | 3.73-3.39 (m, 7H), 3.17 (m, 1H), 2.96 (m, 1H), |
| | | 2.72 (m, 1H), 2.37-2.13 (m, 2H), 1.95-1.74 (m, |
| | | 3H), 1.61-1.39 (m, 8H); LRMS (ES) m/z 494.7 |
| | | (M⁺ + 1). |
| 143 | (S)-2-((7-amino-2-(furan-2-yl)- | ¹H NMR (400 MHz, DMSO-d₆) δ 8.55-8.05 (m, |
| | [1,2,4]triazolo[1,5-a][1,3,5]triazin-5- | 2H), 7.92-7.82 (m, 1H), 7.54-7.26 (m, 1H), |
| | yl)amino)-1-(4-butylpiperazin-1-yl)-3- | 7.12-7.02 (m, 1H), 6.74-6.61 (m, 1H), 4.81- |
| | methylbutan-1-one | 4.59 (m, 1H), 3.83-3.40 (m, 4H), 2.45-2.14 |
| | | (m, 6H), 2.07 (h, J = 6.8 Hz, 1H), 1.48-1.33 (m, |
| | | 2H), 1.33-1.19 (m, 2H), 0.98-0.80 (m, 9H); |
| | | LRMS (ES) m/z 442.7 (M⁺ + 1). |
| 144 | (S)-2-((7-amino-2-(furan-2-yl)- | ¹H NMR (400 MHz, DMSO-d₆) δ 8.45-7.99 (m, |
| | [1,2,4]triazolo[1,5-a][1,3,5]triazin-5- | 2H), 7.89-7.81 (m, 1H), 7.57-7.30 (m, 1H), |
| | yl)amino)-3-methyl-1-(4-(2,2,2- | 7.10-6.99 (m, 1H), 6.71-6.64 (m, 1H), 4.78- |
| | trifluoroethyl)piperazin-1-yl)butan-1- | 4.56 (m, 1H), 3.83-3.66 (m, 2H), 3.66-3.43 |
| | one | (m, 2H), 3.31-3.13 (m, 2H), 2.87-2.72 (m, |
| | | 1H), 2.71-2.55 (m, 3H), 2.15-2.01 (m, 1H), |
| | | 1.05-0.72 (m, 6H); LRMS (ES) m/z 468.6 (M⁺ + |
| | | 1). |
| 145 | 4-((7-amino-2-(furan-2-yl)- | ¹H NMR (400 MHz, DMSO-d₆) δ 8.19 (s, 2H), |
| | [1,2,4]triazolo[1,5-a][1,3,5]triazin-5- | 7.87 (ddd, J = 2.8, 1.7, 0.8 Hz, 1H), 7.09-7.02 |
| | yl)-L-prolyl)-1-methylpiperazin-2-one | (m, 1H), 6.68 (td, J = 3.6, 1.8 Hz, 1H), 5.09- |
| | | 4.82 (m, 1H), 4.51-4.18 (m, 1H), 4.15-4.01 (m, |
| | | 2H), 3.95 (d, J = 12.3 Hz, 1H), 3.88 (d, J = 8.9 |
| | | Hz, 1H), 3.78 (dd, J = 8.0, 4.0 Hz, 1H), 3-71- |
| | | 3.50 (m, 3H), 3.42 (d, J = 6.7 Hz, 2H), 2.27 (td, |
| | | J = 8.0, 3.9 Hz, 1H), 1.92 (s, 3H). m/z 412.6 (M⁺ + |
| | | 1). |
| 146 | (S)-(1-(7-amino-2-(furan-2-yl)- | ¹H NMR (400 MHz, DMSO-d₆) δ 8.27 (d, J = |
| | [1,2,4]triazolo[1,5-a][1,3,5]triazin-5- | 125.6 Hz, 2H), 7.87 (ddd, J = 3.3, 1.8, 0.8 Hz, |
| | yl)pyrrolidin-2-yl)(4- | 1H), 7.12-7.01 (m, 1H), 6.68 (ddd, J = 4.1, 3.4, |
| | methoxypiperidin-1-yl)methanone | 1.8 Hz, 1H), 5.01 (dd, J = 13.5, 9.1 Hz, 1H), 3.90- |
| | | 3.56 (m, 4H), 3.44 (ddt, J = 10.6, 6.2, 2.6 Hz, |
| | | 2H), 3.31-3.27 (m, 3H), 3.25-3.02 (m, 1H), |
| | | 2.34-2.20 (m, 1H), 2.14-1.98 (m, 1H), 1.91 (t, |
| | | J = 6.4 Hz, 2H), 1.86-1.73 (m, 3H), 1.52-1.21 |
| | | (m, 2H). LRMS (ES) m/z 413.6 (M⁺ + 1). |
| 147 | (S)-2-((7-amino-2-(furan-2-yl)- | ¹H NMR (400 MHz, DMSO-d₆) δ 8.28 (s, 1H), |
| | [1,2,4]triazolo[1,5-a][1,3,5]triazin-5- | 7.87 (d, J = 1.8 Hz, 1H), 7.50-7.26 (m, 1H), 7.07 |
| | yl)amino)-2-cyclohexyl-1-(4-(2- | (d, J = 3.4 Hz, 1H), 6.68 (dd, J = 3.4, 1.8 Hz, 1H), |
| | fluoro-2-methylpropyl)piperazin-1- | 4.83-4.64 (m, 1H), 3.71 (q, J = 7.7, 5.1 Hz, 2H), |
| | yl)ethan-1-one | 3.53 (d, J = 37.2 Hz, 2H), 2.65 (dq, J = 10.4, 4.8, |
| | | 4.4 Hz, 0H), 2.45 (dd, J = 23.0, 6.2 Hz, 2H), 1.85- |
| | | 1.54 (m, 5H), 1.35 (s, 2H), 1.29 (s, 2H), 1.24- |
| | | 0.91 (m, 4H). LRMS (ES) m/z 500.7 (M⁺ + 1). |
| 148 | (S)-2-((7-amino-2-(furan-2-yl)- | ¹H NMR (400 MHz, DMSO-d₆) δ 8.26 (d, J = |
| | [1,2,4]triazolo[1,5-a][1,3,5]triazin-5- | 33.5 Hz, 2H), 7.87 (dd, J = 1.8, 0.9 Hz, 1H), 7.42 |
| | yl)amino)-2-cyclohexyl-1-(4-(2,2,2- | (dd, J = 68.2, 8.4 Hz, 1H), 7.06 (dd, J = 3.3, 0.9 |
| | trifluoroethyl)piperazin-1-yl)ethan-1- | Hz, 1H), 6.68 (dd, J = 3.4, 1.8 Hz, 1H), 4.72 (dt, |
| | one | J = 19.0, 8.5 Hz, 1H), 3.74 (d, J = 18.8 Hz, 2H), |
| | | 3.58 (d, J = 5.5 Hz, 1H), 3.49 (d, J = 5.8 Hz, 2H), |
| | | 3.23 (q, J = 10.0 Hz, 2H), 2.81 (s, 1H), 2.71-2.54 |
| | | (m, 5H), 1.86-1.51 (m, 7H), 1.27-0.93 (m, 8H). |
| | | LRMS (ES) m/z 508.6 (M⁺ + 1). |
| 149 | (S)-(1-(7-amino-2-(furan-2-yl)- | ¹H NMR (400 MHz, DMSO-d₆) δ 8.18 (s, 2H), |
| | [1,2,4]triazolo[1,5-a][1,3,5]triazin-5- | 7.91-7.82 (m, 1H), 7.65-7.48 (m, 2H), 7.34- |
| | yl)pyrrolidin-2-yl)(4-(2-fluoro-4- | 7.21 (m, 1H), 7.02 (dt, J = 36.2, 2.4 Hz, 1H), 6.67 |
| | (trifluoromethyl)phenyl)piperazin-1- | (ddt, J = 13.6, 3.6, 1.9 Hz, 1H), 5.05 (dd, J = 16.0, |
| | yl)methanone | 8.2 Hz, 1H), 4.11 (qd, J = 5.3, 1.8 Hz, 1H), 3.90 |
| | | (s, 1H), 3.77 (t, J = 16.4 Hz, 1H), 3.47 (d, J = 17.5 |
| | | Hz, 1H), 3.25-3.05 (m, 4H), 2.30 (d, J = 9.7 Hz, |
| | | 1H), 1.95 (dt, J = 14.4, 7.9 Hz, 3H). LRMS (ES) |
| | | m/z 546.6 (M⁺ + 1). |
| 150 | (S)-(1-(7-amino-2-(furan-2-yl)- | ¹H NMR (400 MHz, DMSO-d₆) δ 8.20 (s, 2H), |
| | [1,2,4]triazolo[1,5-a][1,3,5]triazin-5- | 7.94-7.85 (m, 2H), 7.75-7.59 (m, 2H), 7.43- |
| | yl)pyrrolidin-2-yl)(4-(2- | 7.34 (m, 1H), 7.08 (dd, J = 3.4, 0.8 Hz, 1H), 6.69 |
| | (trifluoromethyl)phenyl)piperazin-1- | (ddd, J = 10.2, 3.4, 1.8 Hz, 1H), 5.03 (ddd, J = |
| | yl)methanone | 8.5, 5.3, 2.7 Hz, 1H), 3.96-3.57 (m, 5H), 3.06- |
| | | 2.74 (m, 4H), 2.28 (td, J = 8.3, 4.3 Hz, 1H), 2.03- |
| | | 1.85 (m, 3H). LRMS (ES) m/z 528.6 (M⁺ + 1). |
| 151 | (S)-(1-(7-amino-2-(furan-2-yl)- | ¹H NMR (400 MHz, DMSO-d₆) δ 8.31 (d, J = |
| | [1,2,4]triazolo[1,5-a][1,3,5]triazin-5- | 121.0 Hz, 2H), 7.86 (ddd, J = 7.1, 1.8, 0.8 Hz, |
| | yl)pyrrolidin-2-yl)(4- | 1H), 7.25 (ddd, J = 8.5, 7.2, 3.0 Hz, 2H), 7.08- |
| | phenylpiperazin-1-yl)methanone | 6.93 (m, 3H), 6.86-6.78 (m, 1H), 6.67 (ddd, J = |
| | | 10.0, 3.4, 1.8 Hz, 1H), 5.05 (ddd, J = 12.1, 8.8, |
| | | 2.9 Hz, 1H), 4.14 (q, J = 5.2 Hz, 3H), 3.87-3.48 |
| | | (m, 8H), 2.35-2.23 (m, 1H), 2.01-1.80 (m, |
| | | 3H). LRMS (ES) m/z 460.6(M⁺ + 1). |
| 162 | (S)-2-((7-amino-2-(furan-2-yl)- | ¹H NMR (400 MHz, DMSO-d₆) δ 8.27 (s, 2H), |
| | [1,2,4]triazolo[1,5-a][1,3,5]triazin-5- | 7.88 (dd, J = 1.9, 0.9 Hz, 1H), 7.52 (m, 1H), 7.29- |
| | yl)amino)-1-(4-(2,4- | 6.96 (m, 4H), 6.68 (dd, J = 3.4, 1.8 Hz, 1H), |
| | difluorophenyl)piperazin-1-yl)butan- | 4.83 (m, 1H), 3-92-3.50 (m, 4H), 3.26-2.83 |
| | 1-one | (m, 4H), 1.72 (m, 2H), 0.93 (t, J = 7.3 Hz, 3H); |
| | | LRMS (ES) m/z 484.5 (M⁺ + 1). |
| 163 | (S)-2-((7-amino-2-(furan-2-yl)- | ¹H NMR (400 MHz, DMSO-d₆) δ 8.32 (s, 2H), |
| | [1,2,4]triazolo[1,5-a][1,3,5]triazin-5- | 7.93 (d, J = 1.9 Hz, 1H), 7.56 (m, 1H), 7.33-6.99 |
| | yl)amino)-1-(4-(2,4- | (m, 4H), 6.74 (dd, J = 3.4, 1.8 Hz, 1H), 4.94 (m, |
| | difluorophenyl)piperazin-1-yl)pentan- | 1H), 4.01-3.53 (m, 4H), 3.30-2.85 (m, 4H), |
| | 1-one | 1.73 (m, 2H), 1.45 (m, 2H), 0.96 (t, J = 7.3 Hz, |
| | | 3H); LRMS (ES) m/z 498.5 (M⁺ + 1). |
| 164 | (S)-2-((7-amino-2-(furan-2-yl)- | ¹H NMR (400 MHz, DMSO-d₆) δ 8.27 (m, 2H), |
| | [1,2,4]triazolo[1,5-a][1,3,5]triazin-5- | 7.90-7.84 (s, 1H), 7.50 (m, 1H), 7.27-6.93 (m, |
| | yl)amino)-1-(4-(2,4- | 4H), 6.68 (td, J = 3.4, 1.7 Hz, 1H), 4.88 (m, 1H), |
| | difluorophenyl)piperazin-1-yl)hexan- | 3.97-3.42 (m, 4H), 3.24-2.81 (m, 4H), 1.69 (s, |
| | 1-one | 2H), 1.33 (s, 4H), 0.87 (t, J = 6.9 Hz, 3H); LRMS |
| | | (ES) m/z 512.5 (M⁺ + 1). |
| 165 | (S)-2-((7-amino-2-(furan-2-yl)- | ¹H NMR (400 MHz, DMSO-d₆) δ 8.29 (m, 2H), |
| | [1,2,4]triazolo[1,5-a][1,3,5]triazin-5- | 7.88 (s, 1H), 7.29-6.83 (m, 5H), 6.69 (dd, J = |
| | yl)amino)-1-(4-(2,4- | 3.5, 1.8 Hz, 1H), 4.98 (d, J = 8.8 Hz, 1H), 4.05- |
| | difluorophenyl)piperazin-1-yl)-3,3- | 3.44 (m, 4H), 3.26-2.74 (m, 4H), 1.04 (s, 9H); |
| | dimethylbutan-1-one | LRMS (ES) m/z 512.5 (M⁺ + 1). |
| 166 | (2S)-2-((7-amino-2-(furan-2-yl)- | ¹H NMR (400 MHz, DMSO-d₆) δ 8.22 (m, 2H), |
| | [1,2,4]triazolo[1,5-a][1,3,5]triazin-5- | 7.91-7.83 (m, 1H), 7.50 (m, 1H), 7.26-6.90 |
| | yl)amino)-1-(4-(2,4- | (m, 4H), 6.68 (ddd, J = 5.4, 3-4, 1.7 Hz, 1H), 4.77 |
| | difluorophenyl)piperazin-1-yl)-3- | (m, 1H), 4.07-3.49 (m, 4H), 3.27-2.78 (m, |
| | methylpentan-1-one | 4H), 1.92 (m, 1H), 1.58 (m, 1H), 1.29-1.17 (m, |
| | | 1H), 0.93-0.78 (m, 6H); LRMS (ES) m/z 512.5 |
| | | (M⁺ + 1). |
| 167 | (S)-2-((7-amino-2-(furan-2-yl)- | ¹H NMR (400 MHz, DMSO-d₆) δ 8.26 (m, 2H), |
| | [1,2,4]triazolo[1,5-a][1,3,5]triazin-5- | 7.87 (t, J = 2.1 Hz, 1H), 7.60 (m, 1H), 7.27-6.94 |
| | yl)amino)-1-(4-(2,4- | (m, 4H), 6.68 (td, J = 3.7, 1.8 Hz, 1H), 4.97 (m, |
| | difluorophenyl)piperazin-1-yl)-4- | 1H), 3.97-3.45 (m, 4H), 3.26-2.79 (m, 4H), |
| | methylpentan-1-one | 1.77-1.59 (m, 2H), 1.46 (m, 1H), 0.92 (dd, J = |
| | | 6.4, 4.7 Hz, 6H); LRMS (ES) m/z 512.5 (M⁺ + 1). |
| 168 | (S)-2-((7-amino-2-(furan-2-yl)- | ¹H NMR (400 MHz, DMSO-d₆) δ 8.35 (m, 2H), |
| | [1,2,4]triazolo[1,5-a][1,3,5]triazin-5- | 7.93 (d, J = 1.9 Hz, 1H), 7.68 (m, 1H), 7.34-6.98 |
| | yl)amino)-2-cyclopropyl-1-(4-(2,4- | (m, 4H), 6.74 (dd, J = 3.4, 1.8 Hz, 1H), 4.51 (m, |
| | difluorophenyl)piperazin-1-yl)ethan- | 1H), 3.98-3.53 (m, 4H), 3.35-2.97 (m, 4H), |
| | 1-one | 1.38-1.25 (m, 2H), 0.59-0.36 (m, 4H); LRMS |
| | | (ES) m/z 496.6 (M⁺ + 1). |
| 169 | (S)-2-((7-amino-2-(furan-2-yl)- | ¹H NMR (400 MHz, DMSO-d₆) δ 8.24 (m, 2H), |
| | [1,2,4]triazolo[1,5-a][1,3,5]triazin-5- | 7.88 (t, J = 3.1 Hz, 1H), 7.50 (m, 1H), 7.27-6.93 |
| | yl)amino)-2-cyclobutyl-1-(4-(2,4- | (m, 4H), 6.72-6.63 (m, 1H), 4.95 (m, 1H), 4.01- |
| | difluorophenyl)piperazin-1-yl)ethan- | 3.47 (m, 4H), 3.26-2.71 (m, 5H), 2.01-1.67 |
| | 1-one | (m, 6H); LRMS (ES) m/z 510.6 (M⁺ + 1). |
| 170 | (S)-2-((7-amino-2-(furan-2-yl)- | ¹H NMR (400 MHz, DMSO-d₆) δ 8.31 (m, 2H), |
| | [1,2,4]triazolo[1,5-a][1,3,5]triazin-5- | 7.97-7.86 (m, 1H), 7.68 (m, 1H), 7.32-6.94 |
| | yl)amino)-2-cyclopentyl-1-(4-(2,4- | (m, 4H), 6.73 (m, 1H), 4.86-4.72 (m, 1H), 4.09- |
| | difluorophenyl)piperazin-1-yl)ethan- | 3.52 (m, 4H), 3.30-2.79 (m, 4H), 2.43-2.31 |
| | 1-one | (m, 1H), 1.84 (m, 1H), 1.60 (d, J = 44.4 Hz, 5H), |
| | | 1.41-1.27 (m, 2H); LRMS (ES) m/z 524.6 (M⁺ + |
| | | 1). |
| 171 | (S)-2-((7-amino-2-(furan-2-yl)- | ¹H NMR (400 MHz, DMSO-d₆) δ 8.32 (m, 2H), |
| | [1,2,4]triazolo[1,5-a][1,3,5]triazin-5- | 7.92-7.82 (m, 1H), 7.56 (m, 1H), 7.26-6.94 |
| | yl)amino)-1-(4-(2,4- | (m, 4H), 6.68 (dt, J = 5.1, 2.5 Hz, 1H), 4.86- |
| | difluorophenyl)piperazin-1-yl)-2- | 4.67 (m, 1H), 4.03-3.52 (m, 6H), 3.25 (m, 2H), |
| | (tetrahydro-2H-pyran-4-yl)ethan-1- | 3.06-2.77 (m, 4H), 2.12-2.00 (m, 1H), 1.72 |
| | one | (m, 1H), 1.52-1.20 (m, 3H); LRMS (ES) m/z |
| | | 540.7 (M⁺ + 1). |
| 172 | (S)-2-((7-amino-2-(furan-2-yl)- | ¹H NMR (400 MHz, DMSO-d₆) δ 8.27 (m, 2H), |
| | [1,2,4]triazolo[1,5-a][1,3,5]triazin-5- | 7.87 (d, J = 1.7 Hz, 1H), 7.37 (m, 1H), 7.07 (dd, J = |
| | yl)amino)-1-(4-butylpiperazin-1- | 5.5, 3.3 Hz, 1H), 6.67 (dd, J = 3.4, 1.8 Hz, 1H), |
| | yl)butan-1-one | 4.79 (m, 1H), 3.59 (m, 4H), 2.48-2.03 (m, 6H), |
| | | 1.80-1.53 (m, 2H), 1.45-1.34 (m, 2H), 1.27 (m, |
| | | 2H), 0.89 (m, 6H); LRMS (ES) m/z 428.6 (M⁺ + |
| | | 1). |
| 173 | (S)-2-((7-amino-2-(furan-2-yl)- | ¹H NMR (400 MHz, DMSO-d₆) δ 8.26 (m, 2H), |
| | [1,2,4]triazolo[1,5-a][1,3,5]triazin-5- | 7.87 (d, J = 2.0 Hz, 1H), 7.39 (m, 1H), 7.07 (t, J = |
| | yl)amino)-1-(4-butylpiperazin-1- | 4.2 Hz, 1H), 6.68 (dt, J = 3.6, 1.7 Hz, 1H), 4.85 |
| | yl)hexan-1-one | (m, 1H), 3.67-3.37 (m, 4H), 2.46-2.08 (m, |
| | | 6H), 1.74-1.53 (m, 2H), 1.41 (m, 2H), 1.35- |
| | | 1.23 (m, 6H), 0.87 (m, 6H); LRMS (ES) m/z |
| | | 456.6 (M⁺ + 1). |
| 174 | (S)-2-((7-amino-2-(furan-2-yl)- | ¹H NMR (400 MHz, DMSO-d₆) δ 8.30 (m, 2H), |
| | [1,2,4]triazolo[1,5-a][1,3,5]triazin-5- | 7.87 (d, J = 1.6 Hz, 1H), 7.50 (m, 1H), 7.07 (d, J = |
| | yl)amino)-1-(4-butylpiperazin-1-yl)- | 3.3 Hz, 1H), 6.68 (dd, J = 3.4, 1.8 Hz, 1H), 4.75 |
| | 3,3-dimethylbutan-1-one | (m, 1H), 3.80-3.38 (m, 4H), 2.29 (m, 6H), 1.89 |
| | | (m, 1H), 1.53 (m, 1H), 1.40 (m, 2H), 1.33-1.11 |
| | | (m, 3H), 0.85 (m, 9H); LRMS (ES) m/z 456.6 |
| | | (M⁺ + 1). |
| 175 | (S)-2-((7-amino-2-(furan-2-yl)- | ¹H NMR (400 MHz, DMSO-d₆) δ 8.25 (m, 2H), |
| | [1,2,4]triazolo[1,5-a][1,3,5]triazin-5- | 7.87 (d, J = 1.6 Hz, 1H), 7.50 (m, 1H), 7.07 (d, J = |
| | yl)amino)-1-(4-butylpiperazin-1-yl)-4- | 3.3 Hz, 1H), 6.68 (dd, J = 3-4, 1.8 Hz, 1H), 4.93 |
| | methylpentan-1-one | (m, 1H), 3.52 (m, 4H), 2.48-2.12 (m, 6H), 1.65 |
| | | (m, 2H), 1.48-1.33 (m, 3H), 1.33-1.23 (m, |
| | | 2H), 0.95-0.82 (m, 9H); LRMS (ES) m/z 456.6 |
| | | (M⁺ + 1). |
| 176 | (S)-2-((7-amino-2-(furan-2-yl)- | ¹H NMR (400 MHz, DMSO-d₆) δ 8.29 (m, 2H), |
| | [1,2,4]triazolo[1,5-a][1,3,5]triazin-5- | 7.87 (dd, J = 1.8, 0.8 Hz, 1H), 7.44 (m, 1H), 7.06 |
| | yl)amino)-1-(4-butylpiperazin-1-yl)-2- | (dd, J = 7.3, 3.3 Hz, 1H), 6.67 (dd, J = 3.4, 1.8 |
| | cyclopropylethan-1-one | Hz, 1H), 4.46 (dt, J = 14.3, 7.9 Hz, 1H), 3.53 (m, |
| | | 4H), 2.31 (mj, 6H), 1.50-1.35 (m, 2H), 1.35- |
| | | 1.13 (m, 3H), 0.87 (t, J = 7.3 Hz, 3H), 0.52-0.28 |
| | | (m, 4H); LRMS (ES) m/z 440.6 (M⁺ + 1). |
| 177 | (S)-2-((7-amino-2-(furan-2-yl)- | ¹H NMR (400 MHz, DMSO-d₆) δ 8.22 (s, 2H), |
| | [1,2,4]triazolo[1,5-a][1,3,5]triazin-5- | 7.87 (d, J = 1.8 Hz, 1H), 7.34 (m, 1H), 7.07 (d, J = |
| | yl)amino)-1-(4-butylpiperazin-1-yl)-2- | 3.3 Hz, 1H), 6.68 (dd, J = 3.4, 1.9 Hz, 1H), 4.91 |
| | cyclobutylethan-1-one | (dt, J = 16.1, 8.4 Hz, 1H), 3.55 (m, 4H), 2.84- |
| | | 2.61 (m, 1H), 2.27 (m, 6H), 2.03-1.57 (m, 6H), |
| | | 1.53-0.99 (m, 4H), 0.88 (t, J = 7.3 Hz, 3H); |
| | | LRMS (ES) m/z 454.6 (M⁺ + 1). |
| 178 | (S)-2-((7-amino-2-(furan-2-yl)- | ¹H NMR (400 MHz, DMSO-d₆) δ 8.26 (m, 2H), |
| | [1,2,4]triazolo[1,5-a][1,3,5]triazin-5- | 7.87 (d, J = 1.6 Hz, 1H), 7.47 (m, 1H), 7.06 (d, J = |
| | yl)amino)-1-(4-butylpiperazin-1-yl)-2- | 3.3 Hz, 1H), 6.68 (dd, J = 3.5, 1.9 Hz, 1H), 4.75 |
| | cyclopentylethan-1-one | (m, 1H), 3.79-3.40 (m, 4H), 2.29 (m, 6H), 1.73 |
| | | (s, 1H), 1.66-1.07 (m, 12H), 0.87 (t, J = 7.3 Hz, |
| | | 3H); LRMS (ES) m/z 468.6 (M⁺ + 1). |
| 187 | (S)-2-((7-amino-2-(furan-2-yl)- | ¹H NMR (400 MHz, Chloroform-d) δ 9.72 (s, |
| | [1,2,4]triazolo[1,5-a][1,3,5]triazin-5- | 1H), 8.44 (d, J = 9.5 Hz, 1H), 7.57 (dd, J = 1.8, |
| | yl)amino)-1-(4-butylpiperazin-1-yl)-2- | 0.8 Hz, 1H), 7.17 (dd, J = 3.5, 0.8 Hz, 1H), 6.54 |
| | cyclohexylethan-1-one | (dd, J = 3.4, 1.8 Hz, 1H), 6.34 (s, 1H), 5.16 (t, J = |
| | | 9.4 Hz, 1H), 4.09 (dd, J = 11.7, 6.6 Hz, 1H), 3.82 |
| | | (ddd, J = 18.4, 12.1, 4.7 Hz, 2H), 3.59 (ddd, J = |
| | | 13.2, 7.8, 3.4 Hz, 1H), 2.63-2.49 (m, 2H), 2.52- |
| | | 2.45 (m, 1H), 2.42-2.28 (m, 3H), 1.79 (dd, J = |
| | | 20.0, 10.4 Hz, 2H), 1.71-1.53 (m, 4H), 1.53- |
| | | 1.41 (m, 2H), 1.38-1.23 (m, 3H), 1.21-0.93 (m, |
| | | 5H), 0.90 (t, J = 7.3 Hz, 3H). LRMS (ES) m/z |
| | | 482.6(M⁺ + 1). |
| 188 | (S)-2-((7-amino-2-(furan-2-yl)- | ¹H NMR (400 MHz, Chloroform-d) δ 9.05 (s, |
| | [1,2,4]triazolo[1,5-a][1,3,5]triazin-5- | 1H), 8.44 (d, J = 8.1 Hz, 1H), 7.47 (d, J = 1.7 Hz, |
| | yl)amino)-2-cyclohexyl-1-(4-(2- | 1H), 6.99 (d, J = 3.4 Hz, 1H), 6.48-6.26 (m, |
| | methoxyethyl)piperazin-1-yl)ethan-1- | 2H), 4.68-4.45 (m, 3H), 4.10 (s, 1H), 3.84 |
| | one | (hept, J = 9.9, 8.5 Hz, 3H), 3.63-3.51 (m, 4H), |
| | | 3.44-3.24 (m, 3H), 3.01 (qd, J = 7.5, 3.0 Hz, |
| | | 3H), 1.82 (d, J = 11.8 Hz, 1H), 1.69-1.47 (m, |
| | | 6H), 1.10-0.98 (m, 3H). LRMS (ES) m/z |
| | | 485.6(M⁺ + 1). |
| 189 | (S)-(1-(7-amino-2-(5-methylfuran-2- | ¹H NMR (400 MHz, DMSO-d₆) δ 8.69-7.98 (m, |
| | yl)-[1,2,4]triazolo[1,5-a][1,3,5]triazin- | 2H), 6.97-6.87 (m, 1H), 6.34-6.23 (m, 1H), |
| | 5-yl)pyrrolidin-2-yl)(4- | 5.04-4.93 (m, 1H), 3.76-3.51 (m, 5H), 3.48- |
| | butylpiperazin-1-yl)methanone | 3.19 (m, 1H), 2.72-2.58 (m, 1H), 2.48-2.40 |
| | | (m, 1H), 2.40-2.11 (m, 8H), 1.97-1.76 (m, 3H), |
| | | 1.51-1.38 (m, 2H), 1.38-1.25 (m, 2H), 0.90 (t, |
| | | J = 7.3 Hz, 3H) ; LRMS (ES) m/z 454.5 (M⁺ + 1). |
| 190 | (S)-2-((7-amino-2-(furan-2-yl)- | ¹H NMR (400 MHz, DMSO-d₆) δ 8.65-8.05 (m, |
| | [1,2,4]triazolo[1,5-a][1,3,5]triazin-5- | 2H), 7.88 (dd, J = 1.9, 0.9 Hz, 1H), 7.66-7.51 |
| | yl)amino)-1-(4-butylpiperazin-1-yl)-2- | (m, 1H), 7.51-7.42 (m, 2H), 7.41-7.34 (m, 2H), |
| | phenylethan-1-one | 7.34-7.26 (m, 1H), 7.12-7.05 (m, 1H), 6.72- |
| | | 6.65 (m, 1H), 6.07-5.95 (m, 1H), 3.80-3.38 |
| | | (m, 4H), 2.46-2.25 (m, 2H), 2.23-2.03 (m, |
| | | 3H), 1.87-1.70 (m, 1H), 1.44-1.29 (m, 2H), |
| | | 1.29-1.16 (m, 2H), 0.84 (t, J = 7.2 Hz, 3H) ; |
| | | LRMS (ES) m/z 476.5 (M⁺ + 1). |
| 191 | (S)-2-((7-amino-2-(furan-2-yl)- | ¹H NMR (400 MHz, DMSO-d₆) δ 8.56-8.02 (m, |
| | [1,2,4]triazolo[1,5-a][1,3,5]triazin-5- | 2H), 7.90-7.84 (m, 1H), 7.79-7.59 (m, 1H), |
| | yl)amino)-1-(4-butylpiperazin-1-yl)-3- | 7.35-7.24 (m, 4H), 7.24-7.15 (m, 1H), 7.11- |
| | phenylpropan-1-one | 7.03 (m, 1H), 6.71-6.64 (m, 1H), 5.15-4.98 (m, |
| | | 1H), 3.63-3.20 (m, 4H), 3.08-2.89 (m, 2H), |
| | | 2.48-2.35 (m, 1H), 2.35-2.25 (m, 1H), 2.25- |
| | | 2.08 (m, 3H), 2.08-1.93 (m, 1H), 1.47-1.31 (m, |
| | | 2H), 1.31-1.18 (m, 2H), 0.92-0.82 (m, 3H) ; |
| | | LRMS (ES) m/z 490.5 (M⁺ + 1). |
| 192 | 4-((7-amino-2-(thiazol-2-yl)- | ¹H NMR (400 MHz, DMSO-d₆) δ 8.69-8.24 (m, |
| | [1,2,4]triazolo[1,5-a][1,3,5]triazin-5- | 2H), 8.07-8.01 (m, 1H), 7.98-7.91 (m, 1H), |
| | yl)-L-prolyl)piperazin-1- | 7.56-7.35 (m, 5H), 5.03 (s, 1H), 4.02-3.36 (m, |
| | yl)(phenyl)methanone | 10H), 2.37-2.15 (m, 1H), 2.02-1.81 (m, 3H) ; |
| | | LRMS (ES) m/z 505.3 (M⁺ + 1). |
| 193 | (S)-(1-(7-amino-2-(thiazol-2-yl)- | ¹H NMR (400 MHz, DMSO-d₆) δ 8.75-8.17 (m, |
| | [1,2,4]triazolo[1,5-a][1,3,5]triazin-5- | 2H), 8.09-8.00 (m, 1H), 7.99-7.88 (m, 1H), |
| | yl)pyrrolidin-2-yl)(4-benzylpiperazin- | 7.41-7.32 (m, 4H), 7.31-7.19 (m, 1H), 5.06- |
| | 1-yl)methanone | 4.93 (m, 1H), 3.74-3.59 (m, 4H), 3.55 (d, J = |
| | | 11.6 Hz, 3H), 3.46-3.35 (m, 1H), 2.76-2.58 |
| | | (m, 1H), 2.47-2.14 (m, 4H), 2.01-1.74 (m, |
| | | 3H) ; LRMS (ES) m/z 491.3 (M⁺ + 1). |
| 194 | (S)-2-((7-amino-2-(thiazol-2-yl)- | ¹H NMR (400 MHz, DMSO-d₆) δ 8.63-8.12 (m, |
| | [1,2,4]triazolo[1,5-a][1,3,5]triazin-5- | 2H), 8.05 (d, J = 3.1 Hz, 1H), 7.96 (d, J = 3.1 Hz, |
| | yl)amino)-1-(4-(2,4- | 1H), 7.74-7.56 (m, 1H), 7.28-7.07 (m, 2H), |
| | difluorophenyl)piperazin-1- | 7.05-6.94 (m, 1H), 5.02-4.85 (m, 1H), 3.88- |
| | yl)propan-1-one | 3.48 (m, 4H), 3.24-2.81 (m, 4H), 1.39-1.27 |
| | | (m, 3H) ; LRMS (ES) m/z 487.3 (M⁺ + 1). |
| 195 | (S)-2-((7-amino-2-(furan-2-yl)- | ¹H NMR (400 MHz, Chloroform-d) δ 8.10 (d, J |
| | [1,2,4]triazolo[1,5-a][1,3,5]triazin-5- | = 8.7 Hz, 1H), 7.58 (s, 1H), 7.36 (d, J = 6.7 Hz, |
| | yl)amino)-1-(4-(2-fluoro-2- | 2H), 7.29 (s, 1H), 7.22 (d, J = 12.8 Hz, 2H), 6.61- |
| | methylpropyl)piperazin-1-yl)-2- | 6.35 (m, 2H), 3.92-3.29 (m, 4H), 2.67-2.15 |
| | phenylethan-1-one | (m, 6H), 1.35 (d, J = 20.9 Hz, 6H). LRMS (ES) |
| | | m/z 494.2 (M⁺ + 1). |
| 198 | 4-((7-amino-2-(5-methylfuran-2-yl)- | ¹H NMR (400 MHz, DMSO-d₆ ) δ 8.35(d, J = |
| | [1,2,4]triazolo[1,5-a][1,3,5]triazin-5- | 103.1 Hz, 2H), 7.57-7.23 (m, 5H), 6.95 (dd, J = |
| | yl)-L-prolyl)-1-phenylpiperazin-2-one | 7.1, 3.2 Hz, 1H), 6.35-6.24 (m, 1H), 5.11-4.85 |
| | | (m, 1H), 4.73-4.22 (m, 2H), 4.20-3.90 (m, |
| | | 2H), 3.90-3.59 (m, 4H), 2.37 (d, J = 4.2 Hz, |
| | | 3H), 2.32 (dd, J = 8.2, 4.3 Hz, 1H), 2.07-1.88 |
| | | (m, 3H). LRMS (ES) m/z 488.4 (M⁺ + 1). |
| 199 | (S)-(1-(7-amino-2-(5-methylfuran-2- | ¹H NMR (400 MHz, Chloroform-d) δ 7.37-7.27 |
| | yl)-[1,2,4]triazolo[1,5-a][1,3,5]triazin- | (m, 2H), 7.07-6.89 (m, 3H), 6.26-6.05 (m, |
| | 5-yl)pyrrolidin-2-yl)(4- | 2H), 5.21-4.90 (m, 1H), 4.05-3.46 (m, 6H), |
| | phenylpiperazin-1-yl)methanone | 3.43-3.02 (m, 3H), 2.42 (d, J = 6.4 Hz, 3H), |
| | | 2.36-1.97 (m, 4H). LRMS (ES) m/z 474.5 (M⁺ + |
| | | 1). |
| 200 | (S)-(1-(7-amino-2-(5-methylfuran-2- | ¹H NMR (400 MHz, DMSO-d₆) δ 8.31(d, J = |
| | yl)-[1,2,4]triazolo[1,5-a][1,3,5]triazin- | 106.7 Hz, 2H), 7.85 (d, J = 8.3 Hz, 1H), 7.74- |
| | 5-yl)pyrrolidin-2-yl)(4-(2- | 7.56 (m, 2H), 7.38 (q, J = 7.7 Hz, 1H), 6.96 (d, J = |
| | (trifluoromethyl)phenyl)piperazin-1- | 3.3 Hz, 1H), 6.30 (ddd, J = 8.7, 3.3, 1.2 Hz, 1H), |
| | yl)methanone | 5.02 (dd, J = 8.7, 3.8 Hz, 1H), 3.94-3.57 (m, |
| | | 5H), 3.46 (d, J = 8.6 Hz, 1H), 3.30 (t, J = 9.0 Hz, |
| | | 1H), 3.08-2.73 (m, 4H), 2.41-2.20 (m, 4H), |
| | | 1.93 (dqd, J = 15.1, 8.5, 7.3, 5.0 Hz, 3H). LRMS |
| | | (ES) m/z 542.4 (M⁺ + 1). |
| 201 | (S)-(1-(7-amino-2-(5-methylfuran-2- | ¹H NMR (400 MHz, DMSO-d₆) δ 8.29(d, J = |
| | yl)-[1,2,4]triazolo[1,5-a][1,3,5]triazin- | 100.7 Hz, 2H), 7.40-6.88 (m, 4H), 6.29 (d, J = |
| | 5-yl)pyrrolidin-2-yl)(4-(2,4- | 3.3 Hz, 1H), 5.17-4.92 (m, 1H), 4.35 (dd, J = |
| | difluorophenyl)piperidin-1- | 113.0, 13.1 Hz, 2H), 3.66 (dtd, J = 25.0, 12.1, 11.5, |
| | yl) methanone | 7.6 Hz, 2H), 3.31-3.14 (m, 1H), 2.70 (td, J = |
| | | 12.4, 10.6, 5.9 Hz, 1H), 2.36 (d, J = 2.4 Hz, 4H), |
| | | 1.87 (ddd, J = 54.2, 26.5, 16.0 Hz, 7H). LRMS |
| | | (ES) m/z 509.1 (M⁺ + 1). |
| 202 | 2-(4-((7-amino-2-(5-methylfuran-2- | ¹H NMR (400 MHz, DMSO-d₆) δ 8.29(d, J = |
| | yl)-[1,2,4]triazolo[1,5-a][1,3,5]triazin- | 110.3 Hz, 2H), 7.21 (dd, J = 5.5, 3.6 Hz, 1H), 6.97- |
| | 5-yl)-L-prolyl)piperazin-1-yl)thiazole | 6.83 (m, 2H), 6.28 (ddd, J = 12.9, 3.3, 1.1 Hz, |
| | | 1H), 5.03 (ddd, J = 16.0, 8.9, 3.0 Hz, 1H), 3.99- |
| | | 3.39 (m, 10H), 2.39-2.25 (m, 4H), 2.01-1.82 |
| | | (m, 3H). LRMS (ES) m/z 481.5 (M⁺ + 1). |
| 203 | (S)-(1-(7-amino-2-(5-methylfuran-2- | ¹H NMR (400 MHz, DMSO-d₆) δ 8.41(dd, J = |
| | yl)-[1,2,4]triazolo[1,5-a][1,3,5]triazin- | 4.8, 2.2 Hz, 3H), 6.92 (dd, J = 22.8, 3.2 Hz, 1H), |
| | 5-yl)pyrrolidin-2-yl)(4-(pyrimidin-2- | 6.68 (dt, J = 6.9, 4.7 Hz, 1H), 6.28 (ddd, J = 12.6, |
| | yl)piperazin-1-yl)methanone | 3.3, 1.2 Hz, 1H), 5.04 (ddd, J = 17.0, 8.9, 3.0 Hz, |
| | | 1H), 4.11-3-59 (m, 9H), 3.54-3.44 (m, 1H), |
| | | 2.39-2.24 (m, 4H), 2.00-1.83 (m, 3H). LRMS |
| | | (ES) m/z 476.3 (M⁺ + 1). |
| 207 | (S)-(1-(7-amino-2-(5-methylfuran-2- | ¹H NMR (400 MHz, DMSO-d₆) δ 8.73-8.03 (m, |
| | yl)-[1,2,4]triazolo[1,5-a][1,3,5]triazin- | 2H), 7.54-7.40 (m, 5H), 6.93 (d, J = 6.5 Hz, |
| | 5-yl)azetidin-2-yl)(4- | 1H), 6.32-6.25 (m, 1H), 5.39-5.07 (m, 1H), |
| | benzoylpiperazin-1-yl)methanone | 4.07-3.92 (m, 2H), 3.89-3.37 (m, 8H), 2.71- |
| | | 2.55 (m, 1H), 2.36 (s, 3H), 2.25-2.11 (m, 1H) ; |
| | | LRMS (ES) m/z 488.3 (M⁺ + 1). |
| 208 | (S)-(1-(7-amino-2-(5-methylfuran-2- | ¹H NMR (400 MHz, DMSO-d₆) δ 8.66-8.00 |
| | yl)-[1,2,4]triazolo[1,5-a][1,3,5]triazin- | (m, 2H), 7.54-7.39 (m, 1H), 7.22 (td, J = 10.1, |
| | 5-yl)azetidin-2-yl)(4-(2,4- | 2.5 Hz, 1H), 7.08 (td, J = 8.4, 2.1 Hz, 1H), 6.96- |
| | difluorobenzyl)piperazin-1- | 6.88 (m, 1H), 6.31-6.26 (m, 1H), 5.24-5.15 (m, |
| | yl)methanone | 1H), 4.06-3.89 (m, 2H), 3.76-3.23 (m, 7H), |
| | | 2.70-2.58 (m, 1H), 2.36 (s, 6H), 2.15-2.02 (m, |
| | | 1H) ; LRMS (ES) m/z 510.3 (M⁺ + 1). |
| 209 | (S)-(1-(7-amino-2-(5-methylfuran-2- | ¹H NMR (400 MHz, DMSO-d₆) δ 8.70-7.98 (m, |
| | yl)-[1,2,4]triazolo[1,5-a][1,3,5]triazin- | 2H), 7.28-7.09 (m, 2H), 7.06-6.85 (m, 2H), |
| | 5-yl)azetidin-2-yl)(4-(2,4- | 6.34-6.24 (m, 1H), 5.35-5.22 (m, 1H), 4.03 (s, |
| | difluorophenyl)piperazin-1- | 2H), 3.88-3.40 (m, 4H), 3.09-2.82 (m, 4H), |
| | yl)methanone | 2.76-2.58 (m, 1H), 2.37 (s, 3H), 2.24-2.12 (m, |
| | | 1H); LRMS (ES) m/z 496.3 (M⁺ + 1). |
| 210 | (S)-(1-(7-amino-2-(5-methylfuran-2- | ¹H NMR (400 MHz, DMSO-d₆) δ 8.73-8.01 (m, |
| | yl)-[1,2,4]triazolo[1,5-a][1,3,5]triazin- | 4H), 6.93 (d, J = 13.8 Hz, 1H), 6.68 (t, J = 4.7 Hz, |
| | 5-yl)azetidin-2-yl)(4-(pyrimidin-2- | 1H), 6.29 (s, 1H), 5.28 (dd, J = 9.1, 5.3 Hz, 1H), |
| | yl)piperazin-1-yl)methanone | 4.07-3.88 (m, 4H), 3.86-3.69 (m, 2H), 3.68- |
| | | 3.37 (m, 4H), 2.73-2.58 (m, 1H), 2.36 (s, 3H), |
| | | 2.25-2.13 (m, 1H) ; LRMS (ES) m/z 462.3 (M⁺+ |
| | | 1). |
| 211 | (S)-2-((7-amino-2-(furan-2-yl)- | ¹H NMR (400 MHz, DMSO-d₆) δ |
| | [1,2,4]triazolo[1,5-a][1,3,5]triazin-5- | 8.30 (s,2H), 7.87 (dd, J = 1.8, 0.9 Hz, 1H), 7.42 (d, |
| | yl)amino)-1-(4-(2,2,2- | J = 7.8 Hz, 1H), 7.14-7.01 (m, 1H), 6.68 (dd, J = |
| | trifluoroethyl)piperazin-1-yl)butan-1- | 3.4, 1.8 Hz, 1H), 4.86-4.67 (m, 1H), 3.57 (d, |
| | one | J = 59.5 Hz, 5H), 3.22 (dd, J = 10.2, 7.6 Hz, 2H), |
| | | 2.68-2.58 (m, 3H), 2.51 (p, J = 1.8 Hz, 2H), 1.82- |
| | | 1.59 (m, 2H), 0.91 (t, J = 7.3 Hz, 3H). LRMS |
| | | (ES) m/z 454.4 (M⁺ + 1). |
| 212 | (S)-2-((7-amino-2-(furan-2-yl)- | ¹H NMR (400 MHz, DMSO-d₆) δ |
| | [1,2,4]triazolo[1,5-a][1,3,5]triazin-5- | 8.30 (s,2H), 7.87 (d, J = 2.0 Hz, 1H), 7.50 (dd, J = |
| | yl)amino)-1-(4-(2,2,2- | 32.4, 7.9 Hz, 1H), 7.07 (d, J = 3.6 Hz, 1H), 6.67 |
| | trifluoroethyl)piperazin-1-yl)pentan- | (dd, J = 3.4, 1.8 Hz, 1H), 4.87 (q, J = 7.3 Hz, 1H), |
| | 1-one | 3.70-3.43 (m, 4H), 3.29-3.16 (m, 2H), 2.84- |
| | | 2.59 (m, 3H), 2.51 (t, J = 1.9 Hz, 1H), 1.64 (q, J = |
| | | 7.2 Hz, 2H), 1.41-1.23 (m, 2H), 0.88 (t, J = 7.3 |
| | | Hz, 4H). LRMS (ES) m/z 468.2 (M⁺ + 1). |
| 213 | (S)-2-((7-amino-2-(furan-2-yl)- | ¹H NMR (400 MHz, DMSO-d₆) δ |
| | [1,2,4]triazolo[1,5-a][1,3,5]triazin-5- | 8.30 (s, 2H),7.87 (dd, J = 1.8, 0.9 Hz, 1H), 7.45 (d, |
| | yl)amino)-1-(4-(2,2,2- | J = 7.9 Hz, 1H), 7.12-7.03 (m, 1H), 6.67 (dt, J = |
| | trifluoroethyl)piperazin-1-yl)hexan-1- | 4.2, 2.0 Hz, 1H), 4.84 (dd, J = 8.2, 6.3 Hz, 1H), |
| | one | 3.73-3.41 (m, 4H), 3.23 (dd, J = 10.1, 6.8 Hz, |
| | | 2H), 2.86-2.57 (m, 3H), 1.66 (s, 2H), 1.36-1.21 |
| | | (m, 4H), 0.88-0.82 (m, 3H). LRMS (ES) m/z |
| | | 482.2 (M⁺ + 1). |
| 214 | (2S)-2-((7-amino-2-(furan-2-yl)- | ¹H NMR (400 MHz, DMSO-d₆) δ 8.31 (s,2H), |
| | [1,2,4]triazolo[1,5-a][1,3,5]triazin-5- | 7.87 (s,1H), 7.53 (dd, J = 35.0, 8.5 Hz, 1H), 7.08 |
| | yl)amino)-3-methyl-1-(4-(2,2,2- | (d, J = 3.4 Hz, 1H), 6.68 (s, 1H), 4.75 (dt, J = |
| | trifluoroethyl)piperazin-1-yl)pentan- | 14.9, 8.7 Hz, 1H), 3-75 (d, J = 14.3 Hz, 2H), 3.63- |
| | 1-one | 3.44 (m, 2H), 3.21 (t, J = 10.0 Hz, 2H), 2.81 (t, |
| | | J = 8.1 Hz, 1H), 2.71-2.62 (m, 1H), 2.57 (s, 2H), |
| | | 1.89 (d, J = 9.5 Hz, 1H), 1.63-1.48 (m, 1H), 1.30- |
| | | 1.11 (m, 1H), 0.95-0.76 (m, 7H). LRMS (ES) |
| | | m/z 482.4 (M⁺ + 1). |
| 215 | (S)-2-((7-amino-2-(furan-2-yl)- | ¹H NMR (400 MHz, DMSO-d₆) δ 8.28 (s,2H), |
| | [1,2,4]triazolo[1,5-a][1,3,5]triazin-5- | 7.87 (dd, J = 1.8, 0.9 Hz, 1H), 7.56 (dd, J = 22.7, |
| | yl)amino)-4-methyl-1-(4-(2,2,2- | 8.1 Hz, 1H), 7.08 (dt, J = 3.4, 1.6 Hz, 1H), 6.68 |
| | trifluoroethyl)piperazin-1-yl)pentan- | (dd, J = 3.4, 1.8 Hz, 1H), 4.92 (dd, J = 8.7, 4.5 |
| | 1-one | Hz, 1H), 3.75-3.40 (m, 5H), 3.28-3.17 (m, |
| | | 3H), 2.84-2.63 (m, 2H), 2.52-2.50 (m, 2H), |
| | | 1.66 (ddt, J = 14.5, 9.6, 5.8 Hz, 2H), 1.42 (qd, J = |
| | | 11.7, 9.1, 3.1 Hz, 2H), 0.90 (q, J = 5.0, 4.5 Hz, |
| | | 7H). LRMS (ES) m/z 482.1 (M⁺ + 1). |
| 216 | (S)-2-((7-amino-2-(furan-2-yl)- | ¹H NMR (400 MHz, DMSO-d₆) δ 8.31 (s,2H), |
| | [1,2,4]triazolo[1,5-a][1,3,5]triazin-5- | 7.87 (dd, J = 1.8, 0.8 Hz, 1H), 7.64-7.47 (m, |
| | yl)amino)-2-cyclopropyl-1-(4-(2,2,2- | 1H), 7.10-7.03 (m, 1H), 6.68 (dd, J = 3.4, 1.8 |
| | trifluoroethyl)piperazin-1-yl)ethan-1- | Hz, 1H), 4.45 (t, J = 8.0 Hz, 1H), 3.71-3.42 (m, |
| | one | 5H), 3.29-3.16 (m, 2H), 2.87-2.57 (m, 4H), |
| | | 1.30-1.16 (m, 1H), 0.50-0.33 (m, 4H). LRMS |
| | | (ES) m/z 466.3 (M⁺ + 1). |
| 217 | (S)-2-((7-amino-2-(furan-2-yl)- | ¹H NMR (400 MHz, DMSO-d₆) δ 8.27 (d, J = |
| | [1,2,4]triazolo[1,5-a][1,3,5]triazin-5- | 38.9 Hz, 2H), 7.87 (dd, J = 1.8, 0.9 Hz, 1H), 7.58 |
| | yl)amino)-2-cyclopentyl-1-(4-(2,2,2- | (dd, J = 46.5, 8.2 Hz, 1H), 7.07 (dd, J = 3.4, 0.9 |
| | trifluoroethyl)piperazin-1-yl)ethan-1- | Hz, 1H), 6.68 (dd, J = 3.4, 1.8 Hz, 1H), 4.73 (dt, |
| | one | J = 21.6, 8.8 Hz, 1H), 3.77 (d, J = 18.4 Hz, 2H), |
| | | 3.49 (q, J = 5.3 Hz, 2H), 3.21 (t, J = 10.0 Hz, 2H), |
| | | 2.87-2.77 (m, 1H), 2.56 (d, J = 3.8 Hz, 3H), |
| | | 2.32 (q, J = 8.2 Hz, 1H), 1.75 (t, J = 6.3 Hz, 1H), |
| | | 1.66-1.41 (m, 6H), 1.37-1.16 (m, 2H). LRMS |
| | | (ES) m/z 494.1 (M⁺ + 1). |
| 218 | (S)-(1-(7-amino-2-(5-methylfuran-2- | ¹H NMR (400 MHz, DMSO-d₆) δ 8.70-8.01 (m, |
| | yl)-[1,2,4]triazolo[1,5-a][1,3,5]triazin- | 2H), 7.40-7.22 (m, 5H), 6.94 (s, 1H), 6.34- |
| | 5-yl)azetidin-2-yl)(4-benzylpiperazin- | 6.25 (m, 1H), 5.25-5.14 (m, 1H), 4.05-3.89 |
| | 1-yl)methanone | (m, 2H), 3.75-3.36 (m, 7H), 2.70-2.58 (m, |
| | | 1H), 2.47-2.39 (m, 1H), 2.38-2.17 (m, 5H), |
| | | 2.15-2.03 (m, 1H); LRMS (ES) m/z 474.4 (M⁺ + |
| | | 1). |
| 219 | (S)-(1-(7-amino-2-(5-methylfuran-2- | ¹H NMR (400 MHz, DMSO-d₆) δ 8.76-7.89 (m, |
| | yl)-[1,2,4]triazolo[1,5-a][1,3,5]triazin- | 2H), 6.93 (d, J = 7.0 Hz, 1H), 6.32-6.25 (m, |
| | 5-yl)azetidin-2-yl)(4- | 1H), 5.26-5.12 (m, 1H), 4.06-3.88 (m, 2H), |
| | cyclohexylpiperazin-1-yl)methanone | 3.75-3.39 (m, 4H), 2.73-2.56 (m, 2H), 2.45- |
| | | 2.19 (m, 7H), 2.16-2.01 (m, 1H), 1.84-1.63 (m, |
| | | 4H), 1.62-1.49 (m, 1H), 1.32-1.14 (m, 4H), 1.14- |
| | | 0.97 (m, 1H) ; LRMS (ES) m/z 466.5 (M⁺ + 1). |
| 220 | (S)-(1-(7-amino-2-(5-methylfuran-2- | ¹H NMR (400 MHz, DMSO-d₆) δ 8.73-8.00 (m, |
| | yl)-[1,2,4]triazolo[1,5-a][1,3,5]triazin- | 2H), 7.01-6.86 (m, 1H), 6.29 (dd, J = 3.2, 1.0 |
| | 5-yl)azetidin-2-yl)(4-(2,2,2- | Hz, 1H), 5.21 (dd, J = 9.1, 5.3 Hz, 1H), 4.07-3.87 |
| | trifluoroethyl)piperazin-1- | (m, 2H), 3.75-3.36 (m, 4H), 3.26 (d, J = 10.0 |
| | yl)methanone | Hz, 2H), 2.84-2.54 (m, 5H), 2.36 (s, 3H), 2.19- |
| | | 2.03 (m, 1H); LRMS (ES) m/z 466.0 (M⁺ + 1). |
| 221 | (S)-(1-(7-amino-2-(5-methylfuran-2- | ¹H NMR (400 MHz, DMSO-d₆) δ 8.71-7.96 (m, |
| | yl)-[1,2,4]triazolo[1,5-a][1,3,5]triazin- | 2H), 6.93 (d, J = 3.2 Hz, 1H), 6.29 (dd, J = 3.2, |
| | 5-yl)azetidin-2-yl)(4-(2- | 1.0 Hz, 1H), 5.20 (dd, J = 9.1, 5.3 Hz, 1H), 4.06- |
| | methoxyethyl)piperazin-1- | 3.88 (m, 2H), 3.73-3.36 (m, 6H), 3.25 (s, |
| | yl) methanone | 3H), 2.71-2.52 (m, 4H), 2.42-2.18 (m, 6H), |
| | | 2.15-2.01 (m, 1H); LRMS (ES) m/z 442.3 (M⁺ + |
| | | 1). |
| 222 | (S)-(1-(7-amino-2-(5-methylfuran-2- | ¹H NMR (400 MHz, DMSO-d₆) δ 8.83-7.87 (m, |
| | yl)-[1,2,4]triazolo[1,5-a][1,3,5]triazin- | 2H), 6.99-6.89 (m, 1H), 6.29 (dd, J = 3.2, 1.0 |
| | 5-yl)azetidin-2-yl)(4-butylpiperazin- | Hz, 1H), 5.20 (dd, J = 9.1, 5.3 Hz, 1H), 4.06- |
| | 1-yl)methanone | 3.86 (m, 2H), 3.75-3.36 (m, 4H), 2.74-2.57 |
| | | (m, 1H), 2.36 (s, 3H), 2.35-1.97 (m, 7H), 1.51- |
| | | 1.37 (m, 2H), 1.37-1.20 (m, 2H), 0.89 (t, J = 7.3 |
| | | Hz, 3H); LRMS (ES) m/z 440.3 (M⁺ + 1). |
| 231 | (S)-2-((7-amino-2-(furan-2-yl)- | ¹H NMR ((400 MHz, DMSO-d₆) δ |
| | [1,2,4]triazolo[1,5-a][1,3,5]triazin-5- | 8.32 (s, 2H), 7.87(d, J = 1.7 Hz, 1H), 7.40-7.03 |
| | yl)amino)-3-hydroxy-1-(4-(2,2,2- | (m, 2H), 6.67 (dd, J = 3.5, 1.8 Hz, 1H), 4.99- |
| | trifluoroethyl)piperazin-1-yl)propan- | 4.93 (m, 2H), 3.75-3.48 (m, 6H), 3.28-3.15 |
| | 1-one | (m, 3H), 2.81-2.54 (m, 5H). LRMS (ES) m/z |
| | | 456.3 (M⁺ + 1). |
| 232 | (S)-2-((7-amino-2-(furan-2-yl)- | ¹H NMR (400 MHz, DMSO-d₆) δ 8.31 (s,2H), |
| | [1,2,4]triazolo[1,5-a][1,3,5]triazin-5- | 7.88 (s, 1H), 7.13 (d, J = 42.8 Hz, 2H), 6.69 (s, |
| | yl)amino)-1-(4-(2-fluoro-2- | 1H), 5.07-4.80 (m, 2H), 3.56 (d, J = 55.5 Hz, |
| | methylpropyl)piperazin-1-yl)-3- | 7H), 2.44 (s, 4H), 1.33 (d, J = 21.5 Hz, 6H). |
| | hydroxypropan-1-one | LRMS (ES) m/z 448.4 (M⁺ + 1). |
| 233 | (S)-2-((7-amino-2-(furan-2-yl)- | ¹H NMR (400 MHz, DMSO-d₆) δ 8.29 (s, 2H), |
| | [1,2,4]triazolo[1,5-a][1,3,5]triazin-5- | 7.88 (d, J = 1.9 Hz, 1H), 7.37-7.15 (m, 8H), 7.08 |
| | yl)amino)-1-(4-benzylpiperazin-1-yl)- | (d, J = 3.4 Hz, 1H), 6.69 (dd, J = 3.5, 1.8 Hz, 1H), |
| | 3-hydroxypropan-1-one | 5.02-4.86 (m, 2H), 3.69-3.48 (m, 9H), 2.43- |
| | | 2.26 (m, 4H). LRMS (ES) m/z 464.4 (M⁺ + 1). |
| 234 | 4-((7-amino-2-(furan-2-yl)- | ¹H NMR (400 MHz, DMSO-d₆) δ 8.35 (s, 1H), |
| | [1,2,4]triazolo[1,5-a][1,3,5]triazin-5- | 7.95-7.24 (m, 7H), 7.11-6.26 (m, 2H), 5.11- |
| | yl)amino)-L-seryl)-1-phenylpiperazin- | 4.90 (m, 1H), 4.59-4.35 (m, 1H), 4.29-4.02 |
| | 2-one | (m, 2H), 3.89-3.57 (m, 4H). LRMS (ES) m/z |
| | | 494.4 (M⁺ + 1). |
| 235 | (S)-2-((7-amino-2-(furan-2-yl)- | ¹H NMR (400 MHz, DMSO-d₆) δ 8.39 (d, J = 4.7 |
| | [1,2,4]triazolo[1,5-a][1,3,5]triazin-5- | Hz, 4H), 7.93-7.82 (m, 1H), 7.44-7.02 (m, |
| | yl)amino)-3-hydroxy-1-(4-(pyrimidin- | 2H), 6.73-6.62 (m, 2H), 5.02 (q, J = 6.8, 6.2 |
| | 2-yl)piperazin-1-yl)propan-1-one | Hz, 2H), 3.85-3.57 (m, 11H). LRMS (ES) m/z |
| | | 452.4 (M⁺ + 1). |
| 236 | 4-((7-amino-2-(furan-2-yl)- | ¹H NMR (400 MHz, DMSO-d₆) δ 8.37 (d, J = |
| | [1,2,4]triazolo[1,5-a][1,3,5]triazin-5- | 116.7 Hz, 2H), 7.96-7.87 (m, 1H), 7.63-7.41 |
| | yl)-L-prolyl)-1-(4- | (m, 2H), 7.27 (td, J = 8.8, 3.6 Hz, 2H), 7.10- |
| | fluorophenyl)piperazin-2-one | 7.02 (m, 1H), 6.70 (ddd, J = 11.9, 3.4, 1.8 Hz, |
| | | 1H), 5.14-4.87 (m, 1H), 4.72-4.25 (m, 2H), |
| | | 4.24-3.79 (m, 4H), 3.78-3.59 (m, 4H), 2.36- |
| | | 2.25 (m, 1H), 2.01-1.91 (m, 2H). LRMS (ES) |
| | | m/z 492.1 (M⁺ + 1). |
| 238 | (S)-(1-(7-amino-2-(furan-2- | ¹H NMR (400 MHz, DMSO-d₆) δ 7.91-7.88 (m, |
| | yl)thiazolo[5,4-d]pyrimidin-5- | 1H), 7.31-6.88 (m, 3H), 6.72 (dd, J = 3.5, 1.8 |
| | yl)pyrrolidin-2-yl)(4-( 2,2,2- | Hz, 1H), 5.06-4.86 (m, 1H), 3.93-3.42 (m, |
| | trifluoroethyl)piperazin-1- | 6H), 3.32-3.16 (m, 3H), 2.95-2.55 (m, 3H), |
| | yl)methanone | 2.30-2.13 (m, 1H), 2.05-1.85 (m, 2H), 1.84- |
| | | 1.75 (m, 1H) ; LRMS (ES) m/z 482.4 (M⁺ + 1). |
| 239 | (S)-(1-(7-amino-2-(furan-2-yl)- | ¹H NMR (400 MHz, DMSO-d₆) δ 8.31 (m, 1H), |
| | [1,2,4]triazolo[1,5-a][1,3,5]triazin-5- | 7.90-7.80 (m, 1H), 7.06 (ddd, J = 5.3, 3.4, 0.8 |
| | yl)pyrrolidin-2-yl)(4-(4,4,4- | Hz, 1H), 6.68 (td, J = 3.3, 1.8 Hz, 1H), 5.04- |
| | trifluorobutyl)piperazin-1- | 4.93 (m, 1H), 3.73-3.50 (m, 2H), 2.72-2.57 |
| | yl) methanone | (m, 1H), 2.40 (m, 1H), 2.28 (m, 2H), 1.96-1.77 |
| | | (m, 1H), 1.75-1.61 (m, 1H). |
| 240 | (S)-2-((7-amino-2-(furan-2-yl)- | ¹H NMR (400 MHz, DMSO-d₆) δ 8.39 (d, J = 4.7 |
| | [1,2,4]triazolo[1,5-a][1,3,5]triazin-5- | Hz, 4H), 7.86 (d, J = 1.8 Hz, 1H), 7.54 (dd, J = |
| | yl)amino)-1-(4-(pyrimidin-2- | 43.1, 7.5 Hz, 1H), 7.06 (dd, J = 13.9, 3.4 Hz, 1H), |
| | yl)piperazin-1-yl)propan-1-one | 6.66 (h, J = 2.5 Hz, 2H), 4.95 (t, J = 7.2 Hz, 1H), |
| | | 3.85-3.51 (m, 8H), 1.32 (d, J = 6.8 Hz, 3H). |
| | | LRMS (ES) m/z 492.1 (M⁺ + 1). |
| 241 | (S)-2-((7-amino-2-(furan-2-yl)- | ¹H NMR (400 MHz, DMSO-d₆) δ 8.38 (d, J = 4.8 |
| | [1,2,4]triazolo[1,5-a][1,3,5]triazin-5- | Hz, 4H), 7.89-7.52 (m, 2H), 7.06 (dd, J = 11.7, |
| | yl)amino)-1-(4-(pyrimidin-2- | 3.4 Hz, 1H), 6.70-6.60 (m, 2H), 4.89-4.68 (m, |
| | yl)piperazin-1-yl)-2-(tetrahydro-2H- | 1H), 3.99-3.46 (m, 11H), 3.29-3.14 (m, 3H), |
| | pyran-4-yl)ethan-1-one | 2.15-2.01 (m, 1H), 1.81-1.66 (m, 1H), 1.54- |
| | | 1.20 (m, 4H). LRMS (ES) m/z 506.3 (M⁺ + 1). |
| 242 | (S)-(1-(7-amino-2-(furan-2-yl)- | ¹H NMR (400 MHz, DMSO-d₆) δ 8.37 (dd, J = |
| | [1,2,4]triazolo[1,5-a][1,3,5]triazin-5- | 25.8, 4.7 Hz, 3H), 7.86 (d, J = 10.2 Hz, 1H), 7.10- |
| | yl)azetidin-2-yl)(4-(pyrimidin-2- | 6.96 (m, 1H), 6.64 (dt, J = 23.0, 4.7 Hz, 3H), |
| | yl)piperazin-1-yl)methanone | 5.28 (dd, J = 9.2, 5.3 Hz, 1H), 4.06-3.90 (m, |
| | | 3H), 3.83-3.49 (m, 9H). LRMS (ES) m/z 448.3 |
| | | (M⁺ + 1). |
| 243 | (S)-(1-(7-amino-2-(5-methylfuran-2- | ¹H NMR (400 MHz, DMSO-d₆) δ 7.31-6.77 (m, |
| | yl)thiazolo[5,4-d]pyrimidin-5- | 3H), 6.34 (dd, J = 3.3, 0.9 Hz, 1H), 5.04-4.89 |
| | yl)pyrrolidin-2-yl)(4-butylpiperazin- | (m, 1H), 3.89-3.41 (m, 5H), 3.29-3.14 (m, |
| | 1-yl)methanone | 2H), 2.46-2.28 (m, 6H), 2.28-2.10 (m, 3H), |
| | | 2.03-1.85 (m, 2H), 1.84-1.73 (m, 1H), 1.53- |
| | | 1.40 (m, 2H), 1.40-1.23 (m, 2H), 0.92 (t, J = |
| | | 6.8 Hz, 3H) ; LRMS (ES) m/z 470.4 (M⁺ + 1). |
| 246 | 4-((7-amino-2-(furan-2-yl)- | ¹H NMR (400 MHz, DMSO-d₆) δ 8.52-8.06 (m, |
| | [1,2,4]triazolo[1,5-a][1,3,5]triazin-5- | 2H), 7.84 (t, J = 2.4 Hz, 1H), 7.07 (dd, J = 10.5, |
| | yl)-L-prolyl)-1-(2- | 3.4 Hz, 1H), 6.66 (dt, J = 4.7, 2.2 Hz, 1H), 4.92 |
| | methoxyethyl)piperazin-2-one | (dddd, J = 39.3, 26.6, 8.8, 3.5 Hz, 1H), 4.54- |
| | | 3.74 (m, 4H), 3.55-3.34 (m, 7H), 2.26 (ddd, J = |
| | | 11.1, 7.2, 3.0 Hz, 1H), 2.04-1.75 (m, 3H). |
| | | LRMS (ES) m/z 456.3 (M⁺ + 1). |
| 247 | 4-((7-amino-2-(furan-2-yl)- | ¹H NMR (400 MHz, DMSO-d₆) δ 7.65-7.54 (m, |
| | [1,2,4]triazolo[1,5-a][1,3,5]triazin-5- | 1H), 7.30 (d, J = 3.7 Hz, 1H), 6.57 (dd, J = 3.6, |
| | yl)-L-prolyl)-1-butylpiperazin-2-one | 1.7 Hz, 1H), 5-45 (d, J = 16.5 Hz, 1H), 4.92 (dd, J = |
| | | 8.5, 2.9 Hz, 1H), 4.39-4.07 (m, 2H), 3.97- |
| | | 3.78 (m, 2H), 3.60-3.20 (m, 5H), 2.58-2.45 |
| | | (m, 1H), 2.26-1.93 (m, 3H), 1.67-1.44 (m, 2H), |
| | | 1.33 (dq, J = 16.4, 9.0, 8.4 Hz, 3H), 0.95 (dt, J = |
| | | 14.5, 7.6 Hz, 3H). LRMS (ES) m/z 454.4 (M⁺ + 1). |
| 248 | (S)-2-((7-amino-2-(furan-2-yl)- | ¹H NMR (400 MHz, DMSO-d₆) δ |
| | [1,2,4]triazolo[1,5-a][1,3,5]triazin-5- | 8.27 (s, 2H), 7.85 (d, J = 18.5 Hz, 1H), 7.52 (d, J = |
| | yl)amino)-1-(4-(thiazol-2- | 7.7 Hz, 1H), 7.27-6.52 (m, 4H), 4.82 (s, 1H), |
| | yl)piperazin-1-yl)butan-1-one | 3.97-3.48 (m, 8H), 1.90-1.42 (m, 2H), 0.91 |
| | | (p, J = 12.5, 9.9 Hz, 3H). LRMS (ES) m/z 455.4 |
| | | (M⁺ + 1). |
| 249 | (S)-2-((7-amino-2-(furan-2-yl)- | ¹H NMR (400 MHz, DMSO-d₆) δ 8.31 (d, J = |
| | [1,2,4]triazolo[1,5-a][1,3,5]triazin-5- | 47.9 Hz, 2H), 7.86 (d, J = 1.9 Hz, 1H), 7.65 (dd, |
| | yl)amino)-2-(tetrahydro-2H-pyran-4- | J = 47.3, 8.2 Hz, 1H), 7.19 (dd, J = 3.7, 1.7 Hz, |
| | yl)-1-(4-(thiazol-2-yl)piperazin-1- | 1H), 7.05 (dd, J = 24.9, 3.4 Hz, 1H), 6.87 (t, J = |
| | yl)ethan-1-one | 3.4 Hz, 1H), 6.66 (dd, J = 3.4, 1.8 Hz, 1H), 4.87- |
| | | 4.70 (m, 1H), 4.16-3.92 (m, 1H), 3.85 (td, J = |
| | | 10.9, 9.9, 3.5 Hz, 3H), 3.78-3.54 (m, 3H), 3.50 |
| | | (q, J = 6.3, 4.8 Hz, 2H), 3.28-3.16 (m, 2H), 2.13- |
| | | 2.00 (m, 1H), 1.80-1.67 (m, 1H), 1.52-1.20 |
| | | (m, 3H). LRMS (ES) m/z 511.3 (M⁺ + 1). |
| 250 | (S)-2-((7-amino-2-(thiazol-2-yl)- | ¹H NMR (400 MHz, DMSO-d₆) δ 8.40 (d, J = |
| | [1,2,4]triazolo[1,5-a][1,3,5]triazin-5- | 36.0 Hz, 2H), 8.04 (dd, J = 3.3, 1.2 Hz, 1H), 7.96 |
| | yl)amino)-2-cyclohexyl-1-(4-(2,2,2- | (t, J = 3.5 Hz, 1H), 7.57 (dd, J = 73.3, 8.4 Hz, 1H), |
| | trifluoroethyl)piperazin-1-yl)ethan-1- | 4.79-4.65 (m, 1H), 3.82-3.41 (m, 4H), 3.24 |
| | one | (q, J = 10.1 Hz, 2H), 2.71-2.54 (m, 3H), 1.86- |
| | | 1.53 (m, 7H), 1.11 (dtt, J = 36.0, 24.4, 14.3 Hz, |
| | | 6H). LRMS (ES) m/z 525.4 (M⁺ + 1). |
| 251 | (S)-2-((7-amino-2-(furan-2-yl)- | ¹H NMR (400 MHz, DMSO-d₆) δ 8.55-8.00 (m, |
| | [1,2,4]triazolo[1,5-a][1,3,5]triazin-5- | 2H), 7.87 (d, J = 0.9 Hz, 1H), 7.70-7.45 (m, 1H), |
| | yl)amino)-1-(4-(thiazol-2- | 7.20 (d, J = 3.6 Hz, 1H), 7.12-6.98 (m, 1H), 6.89 |
| | yl)piperazin-1-yl)propan-1-one | (t, J = 4.0 Hz, 1H), 6.67 (dd, J = 3.3, 1.8 Hz, 1H), |
| | | 5.01-4.81 (m, 1H), 3.92-3.35 (m, 8H), 1.30 (t, |
| | | J = 6.9 Hz, 3H) ; LRMS (ES) m/z 441.2 (M⁺ + 1). |
| 264 | (S)-(1-(7-amino-2-(furan-2-yl)- | ¹H NMR (400 MHz, DMSO-d₆) δ 8.49 (d, J = |
| | [1,2,4]triazolo[1,5-a][1,3,5]triazin-5- | 128.3 Hz, 2H), 7.87 (d, J = 3.9 Hz, 1H), 7.05 (dd, |
| | yl)azetidin-2-yl)(4-(4,4,4- | J = 15.8, 3.5 Hz, 1H), 6.67 (dd, J = 3.4, 1.8 Hz, |
| | trifluorobutyl)piperazin-1- | 1H), 5.36-5.23 (m, 1H), 4.06-3.99 (m, 2H), |
| | yl)methanone | 3.61 (ddh, J = 9.7, 6.7, 3.3 Hz, 2H), 3.26-3.11 |
| | | (m, 4H), 2.69-2.55 (m, 1H), 2.25 (h, J = 5.5, 5.0 |
| | | Hz, 1H), 1.97-1.89 (m, 2H), 1.29-1.23 (m, 6H). |
| | | LRMS (ES) m/z 480.3 (M⁺ 1). |
| 281 | (S)-(1-(7-amino-2-(furan-2-yl)- | ¹H NMR (400 MHz, DMSO-d₆) δ 8.31 (m, 2H), |
| | [1,2,4]triazolo[1,5-a][1,3,5]triazin-5- | 7.93-7.80 (m, 1H), 7.05 (ddd, J = 11.1, 3.4, 0.7 |
| | yl)pyrrolidin-2-yl)(4-(3,3,3- | Hz, 1H), 6.68 (td, J = 3.4, 1.8 Hz, 1H), 5.06- |
| | trifluoropropyl)piperazin-1- | 4.92 (m, 1H), 3.76-3.43 (m, 5H), 3.34 (m, 1H), |
| | yl) methanone | 2.82-2.55 (m, 4H), 2.51 (m, 2H), 2.31 (m, 3H), |
| | | 1.96-1.75 (m, 3H); LRMS (ES) m/z 480.5 (M⁺+ |
| | | 1). |
| 300 | (S)-(1-(7-amino-2-(5-methylfuran-2- | ¹H NMR (400 MHz, DMSO-d₆) δ 8.60-8.10 (m, |
| | yl)-[1,2,4]triazolo[1,5-a][1,3,5]triazin- | 2H), 6.93 (dd, J = 19.4, 3.2 Hz, 1H), 6.30 (d, J = |
| | 5-yl)pyrrolidin-2-yl)(4-(4,4,4- | 3.3 Hz, 1H), 5.01 (dt, J = 8.6, 4.6 Hz, 1H), 3.75- |
| | trifluorobutyl)piperazin-1- | 3.54 (m, 4H), 2.47-2.21 (m, 8H), 2.04-1.81 |
| | yl)methanone | (m, 6H), 1.26 (q, J = 6.7 Hz, 3H). LRMS (ES) |
| | | m/z 508.5 (M⁺+1). |
| 301 | (S)-(1-(7-amino-2-(furan-2-yl)- | ¹H NMR (400 MHz, DMSO-d₆) δ 8.32 (m, 2H), |
| | [1,2,4]triazolo[1,5-a][1,3,5]triazin-5- | 7.89-7.79 (m, 1H), 7.05 (dd, J = 10.9, 3.0 Hz, |
| | yl)pyrrolidin-2-yl)(4-hexylpiperazin- | 1H), 6.68 (dd, J = 4.7, 2.9 Hz, 1H), 5.00 (m, 1H), |
| | 1-yl)methanone | 3.79-3.46 (m, 5H), 3.19-3.02 (m, 1H), 2.39- |
| | | 2.11 (m, 3H), 1.99-1.75 (m, 3H), 1.42 (m, 2H), |
| | | 1.26 (m, 10H), 0.88 (m, 3H); LRMS (ES) m/z |
| | | 468.5 (M⁺ + 1). |
| 302 | (S)-(1-(7-amino-2-(furan-2-yl)- | ¹H NMR (400 MHz, DMSO-d₆) δ 8.30 (m, 2H), |
| | [1,2,4]triazolo[1,5-a][1,3,5]triazin-5- | 7.87 (s, 1H), 7.06 (d, J = 3.3 Hz, 1H), 6.68 (dd, J = |
| | yl)pyrrolidin-2-yl)(4-(2-hydroxy-2- | 3.3, 1.8 Hz, 1H), 5.00 (m, 1H), 4.15 (d, J = 8.6 |
| | methylpropyl)piperazin-1- | Hz, 1H), 3.72-3.48 (m, 5H), 3.26 (m, 1H), 2.80 |
| | yl)methanone | (m, 1H), 2.69-2.55 (m, 2H), 2.42-2.14 (m, |
| | | 4H), 1.95-1.70 (m, 3H), 1.12 (s, 6H); LRMS |
| | | (ES) m/z 456.6 (M⁺ + 1). |
| 304 | (S)-2-((7-amino-2-(furan-2-yl)- | ¹H NMR (400 MHz, DMSO-d₆) δ 8.39 (d, J = 4.7 |
| | [1,2,4]triazolo[1,5-a][1,3,5]triazin-5- | Hz, 4H), 7.87 (d, J = 0.9 Hz, 1H), 7.55 (dd, J = |
| | yl)amino)-3-methoxy-1-(4- | 56.2, 8.1 Hz, 1H), 7.07 (dd, J = 7.9, 3.2 Hz, 1H), |
| | (pyrimidin-2-yl)piperazin-1- | 6.67 (dt, J = 7.7, 3.7 Hz, 2H), 5.16 (dd, J = 13.8, |
| | yl)propan-1-one | 6.3 Hz, 1H), 3.94-3.46 (m, 10H), 3.29 (s, 3H); |
| | | LRMS (ES) m/z 466.5 (M⁺ + 1). |
| 305 | (S)-2-((7-amino-2-(furan-2-yl)- | ¹H NMR (400 MHz, DMSO-d₆) δ 8.34 (s, 2H), |
| | [1,2,4]triazolo[1,5-a][1,3,5]triazin-5- | 7.87 (d, J = 0.9 Hz, 1H), 7.58 (dd, J = 48.5, 8.0 |
| | yl)amino)-3-methoxy-1-(4-(thiazol-2- | Hz, 1H), 7.19 (d, J = 3.5 Hz, 1H), 7.06 (dd, J = |
| | yl)piperazin-1-yl)propan-1-one | 15.6, 3.2 Hz, 1H), 6.89 (d, J = 3.5 Hz, 1H), 6.67 |
| | | (dd, J = 3.3, 1.8 Hz, 1H), 5.13 (m, 1H), 3.60 (m, |
| | | 10H), 3.27 (m, 3H); LRMS (ES) m/z 471.5 (M⁺ + |
| | | 1). |
| 306 | (S)-2-((7-amino-2-(furan-2-yl)- | ¹H NMR (400 MHz, DMSO-d₆) δ 8.33 (m, 2H), |
| | [1,2,4]triazolo[1,5-a][1,3,5]triazin-5- | 7.88 (s, 1H), 7.49 (dd, J = 53.5, 8.1 Hz, 1H), 7.07 |
| | yl)amino)-3-methoxy-1-(4-(2,2,2- | (d, J = 3.1 Hz, 1H), 6.68 (dd, J = 3.2, 1.8 Hz, 1H), |
| | trifluoroethyl)piperazin-1-yl)propan- | 5.09 (dd, J = 14.5, 6.5 Hz, 1H), 3.77-3.38 (m, |
| | 1-one | 6H), 3.30-3.12 (m, 5H), 2.75-2.53 (m, 4H); |
| | | LRMS (ES) m/z 470.5 (M⁺ + 1). |
| 307 | (S)-2-((7-amino-2-(furan-2-yl)- | ¹H NMR (400 MHz, DMSO-d₆) δ 8.38 (m, 4H), |
| | [1,2,4]triazolo[1,5-a][1,3,5]triazin-5- | 7.87 (dd, J = 1.7, 0.8 Hz, 1H), 7.51 (dd, J = 58.4, |
| | yl)amino)-3-ethoxy-1-(4-(pyrimidin- | 8.0 Hz, 1H), 7.07 (dd, J = 7.7, 3.3 Hz, 1H), 6.73- |
| | 2-yl)piperazin-1-yl)propan-1-one | 6.61 (m, 2H), 5.14 (m, 1H), 3.91-3.41 (m, |
| | | 12H), 1.09 (t, J = 7.0 Hz, 3H); LRMS (ES) m/z |
| | | 480.5 (M⁺ + 1). |
| 308 | (S)-2-((7-amino-2-(furan-2-yl)- | ¹H NMR (400 MHz, DMSO-d₆) δ 8.33 (s, 2H), |
| | [1,2,4]triazolo[1,5-a][1,3,5]triazin-5- | 7.88 (s, 1H), 7.45 (dd, J = 54.4, 8.0 Hz, 1H), 7.07 |
| | yl)amino)-3-ethoxy-1-(4-(2,2,2- | (d, J = 3.0 Hz, 1H), 6.68 (s, 1H), 5.07 (dd, J = |
| | trifluoroethyl)piperazin-1-yl)propan- | 14.1, 6.6 Hz, 1H), 3.74-3.38 (m, 8H), 3.24 (dd, |
| | 1-one | J = 19.1, 9.1 Hz, 2H), 2.80-2.55 (m, 4H), 1.09 |
| | | (t, J = 7.0 Hz, 3H); LRMS (ES) m/z 484.4 (M⁺ + |
| | | 1). |
| 309 | (S)-(1-(7-amino-2-(furan-2-yl)- | ¹H NMR (400 MHz, DMSO-d₆) δ 8.35 (m, 2H), |
| | [1,2,4]triazolo[1,5-a][1,3,5]triazin-5- | 7.91-7.77 (m, 1H), 7.11-6.95 (m, 1H), 6.67 (m, |
| | yl)pyrrolidin-2-yl)(2- | 1H), 5.37-3.74 (m, 8H), 3.66 (m, 2H), 2.31 (s, |
| | (trifluoromethyl)-6,7- | 1H), 1.98 (m, 3H); LRMS (ES) m/z 490.4 (M⁺ + |
| | dihydropyrazolo[1,5-a]pyrazin-5(4H)- | 1). |
| | yl)methanone | |
| 310 | (S)-(1-(7-amino-2-(furan-2-yl)- | ¹H NMR (400 MHz, DMSO-d₆) δ 8.32 (m, 2H), |
| | [1,2,4]triazolo[1,5-a][1,3,5]triazin-5- | 7.94-7.78 (m, 1H), 7.10-6.77 (m, 5H), 6.67 (m, |
| | yl)pyrrolidin-2-yl)(4-(4-(2- | 1H), 5.14-4.90 (m, 1H), 4.06-3.93 (m, 2H), |
| | methoxyethoxy)phenyl)piperazin-1- | 3.86-3.39 (m, 8H), 3.32-3.23 (m, 4H), 3.18- |
| | yl)methanone | 2.77 (m, 3H), 2.36-2.19 (m, 1H), 1.91 (m, 3H); |
| | | LRMS (ES) m/z 534.6 (M⁺ + 1). |
| 321 | (S)-(1-(7-amino-2-(furan-2-yl)- | ¹H NMR (400 MHz, DMSO-d₆) δ 8.30 (m, 2H), |
| | [1,2,4]triazolo[1,5-a][1,3,5]triazin-5- | 7.89 (m, 1H), 7.58 (m, 1H), 7.12-7.00 (m, 1H), |
| | yl)pyrrolidin-2-yl)(4-(1-methyl-1H- | 6.69 (s, 1H), 6.63-6.03 (m, 1H), 5.13-4.87 (m, |
| | pyrazol-4-yl)piperazin-1- | 1H), 4.46-4.09 (m, 2H), 3.87-3.52 (m, 5H), |
| | yl)methanone | 3.20 (m, 1H), 2.96-2.61 (m, 2H), 2.39-2.21 |
| | | (m, 1H), 2.17-1.69 (m, 4H), 1.62-1.33 (m, 2H); |
| | | LRMS (ES) m/z 463.5 (M⁺ + 1). |
| 336 | (S)-(1-(5-amino-2-(furan-2-yl)- | ¹H NMR (400 MHz, DMSO-d₆) δ 7.86 (dd, J = |
| | [1,2,4]triazolo[1,5-c]pyrimidin-7- | 1.7, 0.7 Hz, 1H), 7.46 (brs, 2H), 7.09-7.01 (m, |
| | yl)pyrrolidin- 2-yl)(4-(2- | 1H), 6.67 (dd, J = 3.4, 1.8 Hz, 1H), 5.58 (brs, 1H), |
| | methoxyethyl)piperazin-1- | 4.95 (brs, 1H), 3.71-3.41 (m, 8H), 3.25 (s, 4H), |
| | yl)methanone | 2.73-2.61 (m, 1H), 2.56-2.53 (m, 2H), 2.42- |
| | | 2.13 (m, 3H), 2.04-1.73 (m, 3H) ; LRMS (ES) |
| | | m/z 441.6 (M⁺ + 1). |
| 338 | (S)-2-((7-amino-2-(furan-2-yl)- | ¹H NMR (400 MHz, DMSO-d₆) δ 8.49-8.04 |
| | [1,2,4]triazolo[1,5-a][1,3,5]triazin-5- | (m, 2H), 7.91-7.81 (m, 1H), 7.51-7.27 (m, 1H), |
| | yl)amino)-1-(4-(2-fluoro-2- | 7.13-7.01 (m, 1H), 6.68 (dd, J = 3.3, 1.7 Hz, 1H), |
| | methylpropyl)piperazin-1-yl)butan-1- | 4.86-4.67 (m, 1H), 3.76-3.42 (m, 5H), 2.71- |
| | one | 2.58 (m, 1H), 2.47-2.26 (m, 4H), 1.82-1.56 |
| | | (m, 2H), 1.41-1.24 (m, 6H), 0.96-0.78 (m, |
| | | 3H) ; LRMS (ES) m/z 446.5 (M⁺ + 1). |
| 339 | (S)-2-((7-amino-2-(furan-2-yl)- | ¹H NMR (400 MHz, DMSO-d₆) δ 8.52-8.00 |
| | [1,2,4]triazolo[1,5-a][1,3,5]triazin-5- | (m, 2H), 7.92-7.82 (m, 1H), 7.61-7.36 (m, 1H), |
| | yl)amino)-2-cyclopropyl-1-(4-(2- | 7.14-7.00 (m, 1H), 6.68 (dd, J = 3.3, 1.7 Hz, |
| | fluoro-2-methylpropyl)piperazin-1- | 1H), 4.58-4.34 (m, 1H), 3.78-3.42 (m, 5H), |
| | yl)ethan-1-one | 2.74-2.56 (m, 1H), 2.47-2.27 (m, 4H), 1.33 (d, |
| | | J = 21.4 Hz, 6H), 1.26-1.17 (m, 1H), 0.53-0.43 |
| | | (m, 2H), 0.42-0.28 (m, 2H) ; LRMS (ES) m/z |
| | | 458.6 (M⁺ + 1). |
| 346 | (S)-2-((7-amino-2-(furan-2-yl)- | ¹H NMR (400 MHz, DMSO-d₆) δ 8.35 (m, 4H), |
| | [1,2,4]triazolo[1,5-a][1,3,5]triazin-5- | 7.86 (s, 1H), 7.52 (dd, J = 48.5, 7.9 Hz, 1H), 7.06 |
| | yl)amino)-1-(4-(pyrimidin-2- | (dd, J = 14.6, 3.3 Hz, 1H), 6.71-6.61 (m, 2H), |
| | yl)piperazin-1-yl)butan-1-one | 4.83 (m, 1H), 3.94-3.42 (m, 8H), 1.73 (m, 2H), |
| | | 1.00-0.87 (m, 3H); LRMS (ES) m/z 450.6 |
| | | (M⁺ + 1). |
| 349 | (S)-2-((7-amino-2-(furan-2-yl)- | ¹H NMR (400 MHz, DMSO-d₆) δ 8.27 (m, 2H), |
| | [1,2,4]triazolo[1,5-a][1,3,5]triazin-5- | 7.87 (s, 1H), 7.42 (dd, J = 36.7, 7.6 Hz, 1H), 7.07 |
| | yl)amino)-1-(4-(2-hydroxy-2- | (d, J = 3.1 Hz, 1H), 6.68 (dd, J = 3.3, 1.7 Hz, 1H), |
| | methylpropyl)piperazin-1-yl)propan- | 4.93-4.80 (m, 1H), 4.14 (s, 1H), 3.44 (m, 4H), |
| | 1-one | 2.63 (m, 2H), 2.43 (m, 2H), 2.22 (m, 2H), 1.26 |
| | | (m, 3H), 1.10 (s, 6H); LRMS (ES) m/z 430.6 |
| | | (M⁺ + 1). |
| 350 | (S)-(1-(7-amino-2-(furan-2-yl)- | ¹H NMR (400 MHz, DMSO-d₆) δ 8.71-8.03 (m, |
| | [1,2,4]triazolo[1,5-a][1,3,5]triazin-5- | 2H), 7.88 (d, J = 1.0 Hz, 1H), 7.05 (s, 1H), 6.68 |
| | yl)azetidin-2-yl)(4-(2-hydroxy-2- | (dd, J = 3.4, 1.8 Hz, 1H), 5.21 (dd, J = 9.1, 5.3 Hz, |
| | methylpropyl)piperazin-1- | 1H), 4.11 (m, 1H), 4.07-3.90 (m, 2H), 3.43 (m, |
| | yl)methanone | 3H), 3.31-3.19 (m, 1H), 2.78-2.56 (m, 4H), |
| | | 2.43-2.32 (m, 1H), 2.17 (m, 3H), 1.14 (m, 6H); |
| | | LRMS (ES) m/z 442.5 (M⁺ + 1). |
| 351 | (S)-2-((7-amino-2-(furan-2-yl)- | ¹H NMR (400 MHz, DMSO-d₆) δ 8.30 (s, 2H), |
| | [1,2,4]triazolo[1,5-a][1,3,5]triazin-5- | 7.87 (s, 1H), 7.38 (dd, J = 68.1, 8.6 Hz, 1H), 7.08 |
| | yl)amino)-1-(4-(2-hydroxy-2- | (d, J = 3.3 Hz, 1H), 6.68 (dd, J = 3.2, 1.7 Hz, 1H), |
| | methylpropyl)piperazin-1-yl)-3- | 4.69 (m, 1H), 4.13 (s, 1H), 3.76-3.39 (m, 4H), |
| | methylbutan-1-one | 2.69-2.54 (m, 1H), 2.45-2.30 (m, 3H), 2.21 (s, |
| | | 2H), 2.08 (m, 1H), 1.11 (m, 6H), 0.95-0.80 (m, |
| | | 6H); LRMS (ES) m/z 458.5 (M⁺ + 1). |
| 352 | (S)-(1-(5-amino-2-(furan-2-yl)- | ¹H NMR (400 MHz, DMSO-d₆) δ 7.86 (dd, J = |
| | [1,2,4]triazolo[1,5-c]pyrimidin-7- | 1.7, 0.8 Hz, 1H), 7.47 (brs, 2H), 7.06 (dd, J = 3.4, |
| | yl)pyrrolidin- 2-yl)(4-(2-fluoro-2- | 0.7 Hz, 1H), 6.67 (dd, J = 3.4, 1.8 Hz, 1H), 5.59 |
| | methylpropyl)piperazin-1- | (brs, 1H), 4.96 (brs, 1H), 3.86-3.38 (m, 6H), |
| | yl)methanone | 2.82-2.63 (m, 1H), 2.44-2.14 (m, 6H), 2.03- |
| | | 1.89 (m, 2H), 1.89-1.71 (m, 1H), 1.35 (d, J = 21.4 |
| | | Hz, 6H) ; LRMS (ES) m/z 457.6 (M⁺ + 1). |
| 361 | (S)-(1-(5-amino-2-(furan-2-yl)- | ¹H NMR (400 MHz, DMSO-d₆) δ 7.86 (dd, J = |
| | [1,2,4]triazolo[1,5-c]pyrimidin-7- | 1.7, 0.7 Hz, 1H), 7.47 (brs, 2H), 7.06 (dd, J = 3.4, |
| | yl)pyrrolidin- 2-yl)(4-butylpiperazin- | 0.6 Hz, 1H), 6.67 (dd, J = 3.4, 1.8 Hz, 1H), 5.57 |
| | 1-yl)methanone | (brs, 1H), 4.95 (brs, 1H), 3.79-3.40 (m, 5H), |
| | | 2.65-2.55 (m, 1H), 2.47-2.08 (m, 7H), 2.03- |
| | | 1.89 (m, 2H), 1.88-1.73 (m, 1H), 1.50-1.38 (m, |
| | | 2H), 1.37-1.24 (m, 2H), 0.90 (t, J = 7.3 Hz, |
| | | 3H) ; LRMS (ES) m/z 439.1 (M⁺ + 1). |
| 367 | (S)-2-((7-amino-2-(furan-2-yl)- | ¹H NMR (400 MHz, DMSO-d₆) δ 8.27 (m, 2H), |
| | [1,2,4]triazolo[1,5-a][1,3,5]triazin-5- | 7.86 (m, 1H), 7.65-7.40 (m, 1H), 7.06 (m, 1H), |
| | yl)amino)-3-methyl-1-(4-(3,3,3- | 6.68 (m, 1H), 4.76-4.44 (m, 1H), 3.86 (m, 1H), |
| | trifluoropropyl)piperazin-1-yl)butan- | 3.76-3.38 (m, 7H), 2.66-2.28 (m, 4H), 2.06 |
| | 1-one | (m, 1H), 0.99-0.82 (m, 6H); LRMS (ES) m/z |
| | | 482.5 (M⁺ + 1). |
| 375 | (S)-2-((5-amino-2-(furan-2-yl)- | ¹H NMR (400 MHz, DMSO-d₆) δ 7.89-7.83 (m, |
| | [1,2,4]triazolo[1,5-c]pyrimidin-7- | 1H), 7.56 (brs, 2H), 7.05 (d, J = 3.3 Hz, 1H), 6.67 |
| | yl)amino)-1-(4-(2-fluoro-2- | (dd, J = 3.4, 1.8 Hz, 1H), 5.79 (brs, 1H), 4.82 (s, |
| | methylpropyl)piperazin-1-yl)propan- | 1H), 3.70-3.40 (m, 4H), 2.61-2.36 (m, 7H), |
| | 1-one | 1.33 (d, J = 21.5 Hz, 6H), 1.25 (d, J = 6.8 Hz, |
| | | 3H) ; LRMS (ES) m/z 431.4 (M⁺ + 1). |
| 385 | (S)-(5-(7-amino-2-(furan-2-yl)- | ¹H NMR (400 MHz, DMSO-d₆) δ 8.32 (m, 2H), |
| | [1,2,4]triazolo[1,5-a][1,3,5]triazin-5- | 7.88 (s, 1H), 7.06 (t, J = 3.2 Hz, 1H), 6.68 (dd, J = |
| | yl)-5-azaspiro[2.4]heptan-6-yl)(4- | 3.2, 1.8 Hz, 1H), 5.19-4.99 (m, 1H), 3.71- |
| | (2,2,2-trifluoroethyl)piperazin-1- | 3.39 (m, 6H), 3.25 (m), 2.84 (m, 1H), 2.63 (m, |
| | yl)methanone | 3H), 2.38 (m, 1H), 1.79-1.59 (m, 1H), 0.58 (m, |
| | | 4H); LRMS (ES) m/z 492.31 (M⁺ + 1). |
| 386 | (S)-(5-(7-amino-2-(furan-2-yl)- | ¹H NMR (400 MHz, DMSO-d₆) δ 8.28 (m, 2H), |
| | [1,2,4]triazolo[1,5-a][1,3,5]triazin-5- | 7.87 (d, J = 0.9 Hz, 1H), 7.06 (dd, J = 3.4, 0.6 |
| | yl)-5-azaspiro[2.4]heptan-6-yl)(4-(2- | Hz, 1H), 6.68 (dd, J = 3.3, 1.8 Hz, 1H), 5.17- |
| | fluoro-2-methylpropyl)piperazin-1- | 5.02 (m, 1H), 3.71-3.46 (m, 6H), 2.80-2.53 |
| | yl)methanone | (m, 4H), 2.47-2.22 (m, 3H), 1.72 (m, 1H), 1.34 |
| | | (d, J = 21.4 Hz, 6H), 0.67-0.46 (m, 4H); LRMS |
| | | (ES) m/z 484.37 (M⁺ + 1). |
| 387 | (S)-(1-(7-amino-2-(furan-2-yl)- | ¹H NMR (400 MHz, DMSO-d₆) δ 8.29 (m, 2H), |
| | [1,2,4]triazolo[1,5-a][1,3,5]triazin-5- | 7.88 (s, 1H), 7.06 (dd, J = 6.8, 3.3 Hz, 1H), 6.68 |
| | yl)-4,4-dimethylpyrrolidin-2-yl)(4- | (dd, J = 3.3, 1.8 Hz, 1H), 4.98 (dt, J = 19.6, 8.2 |
| | (2,2,2-trifluoroethyl)piperazin-1- | Hz, 1H), 3.77-3.50 (m, 4H), 3.32-3.14 (m, |
| | yl)methanone | 4H), 3.04-2.79 (m, 1H), 2.78-2.53 (m, 3H), |
| | | 2.23-2.10 (m, 1H), 1.59 (m, 1H), 1.08 (d, J = |
| | | 36.5 Hz, 6H); LRMS (ES) m/z 494.37 (M⁺ + 1). |
| 388 | 2-((7-amino-2-(furan-2-yl)- | ¹H NMR (400 MHz, DMSO-d₆) δ 8.33 (s, 2H), |
| | [1,2,4]triazolo[1,5-a][1,3,5]triazin-5- | 7.88 (s, 1H), 7.26 (s, 1H), 7.06 (t, J = 7.8 Hz, 1H), |
| | yl)amino)-1-(4-(2-fluoro-2- | 6.71-6.62 (m, 1H), 4.12 (d, J = 5.8 Hz, 2H), 3.44 |
| | methylpropyl)piperazin-1-yl)ethan-1- | (m, 4H), 3.30 (m, 2H), 2.45 (s, 4H), 1.34 (d, J = |
| | one | 21.5 Hz, 6H); LRMS (ES) m/z 418.32 (M⁺ + 1). |
| 399 | (S)-2-((7-amino-2-(furan-2-yl)- | ¹H NMR (400 MHz, DMSO-d₆) δ 8.31 (s, 2H), |
| | [1,2,4]triazolo[1,5-a][1,3,5]triazin-5- | 7.88 (d, J = 1.8 Hz, 1H), 7.44 (dd, J = 52.1, 8.3 |
| | yl)amino)-1-(4-(2-hydroxy-2- | Hz, 1H), 7.08 (d, J = 3.3 Hz, 1H), 6.68 (dd, J = |
| | methylpropyl)piperazin-1-yl)-3- | 3.5, 1.8 Hz, 1H), 5.14-5.04 (m, 1H), 4.12 (s, 1H), |
| | methoxypropan-1-one | 3.66-3.40 (m, 6H), 3.33 (s, 2H), 2.56 (d, J = |
| | | 6.8 Hz, 2H), 2.48 (s, 2H), 2.20 (d, J = 2.6 Hz, |
| | | 2H), 1.09 (s, 6H). LRMS (ES) m/z 460.2 (M⁺ + |
| | | 1). |
| 401 | (S)-2-((7-amino-2-(furan-2-yl)- | ¹H NMR (400 MHz, DMSO-d₆) δ 8.33 (s, 2H), |
| | [1,2,4]triazolo[1,5-a][1,3,5]triazin-5- | 7.87 (d, J = 1.7 Hz, 1H), 7.46 (dd, J = 45.3, 8.3 |
| | yl)amino)-1-(4-butylpiperazin-1-yl)-3- | Hz, 1H), 7.07 (d, J = 3.3 Hz, 1H), 6.68 (dd, J = |
| | methoxypropan-1-one | 3.6, 1.8 Hz, 1H), 5.10 (q, J = 6.8 Hz, 1H), 3.68- |
| | | 3.40 (m, 6H), 3.34 (s, 1H), 2.47-2.17 (m, 7H), |
| | | 1.46-1.35 (m, 2H), 1.28 (q, J = 7.3 Hz, 2H), 0.87 |
| | | (t, J = 7.3 Hz, 3H). m/z 444.1 (M⁺ + 1). |

### Example 353: Synthesis of Compound 353, (2-(7-amino-2-(furan-2-yl)-[1,2,4]triazolo[1,5-a][1,3,5]triazin-5-yl)pyrazolidin-1-yl)(4-(2,2,2-trifluoroethyl)piperazin-1-yl)methanone

### [Step 1] tert-butyl 2-(4-(2,2,2-trifluoroethyl)piperazin-1-carbonyl)pyrazolidin-1-carboxylate

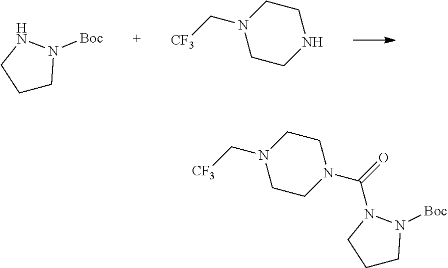 [SMILES: CC(C)(C)OC(=O)N1CCCN1 ; FC(F)(F)CN1CCNCC1 ; CC(C)(C)OC(=O)N1CCCN1C(=O)N1CCN(CC(F)(F)F)CC1]

Tert-butyl pyrazolidin-1-carboxylate (1.000 g, 5.806 mmol), 1,1′-carbonyldiimidazole (CDI, 1.412 g, 8.709 mmol) and N,N-diisopropylethylamine (3.034 mL, 17.419 mmol) were dissolved in dichloromethane (20 mL) at room temperature, after which 1-(2,2,2-trifluoroethyl)piperazine (1.953 g, 11.612 mmol) was added into the resulting solution and stirred at the same temperature for 18 hours. Solvent was removed from the reaction mixture under reduced pressure, after which saturated aqueous solution of sodium hydrogen carbonate solution was poured into the resulting concentrate and an organic layer was extracted with ethyl acetate. The organic layer was washed with saturated aqueous solution of sodium chloride, dehydrated with anhydrous magnesium sulfate, filtered, and concentrated under reduced pressure. The resulting concentrate was purified via column chromatography (SiO₂, 12 g cartridge; ethyl acetate/hexane=0 to 100%) and concentrated to obtain a title compound (0.500 g, 23.5%) as a white solid of a foam type.

### [Step 2] Synthesis of pyrazolidin-1-yl(4-(2,2,2-trifluoroethyl)piperazin-1-yl)methanone

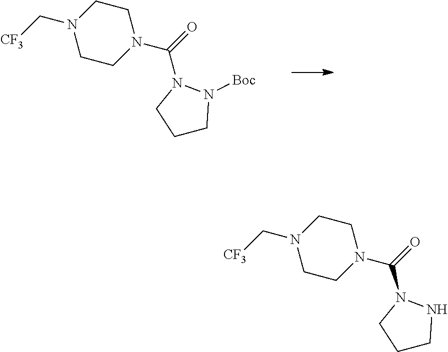

The tert-butyl 2-(4-(2,2,2-trifluoroethyl)piperazin-1-carbonyl)pyrazolidin-1-carboxylate (0.400 g, 1.092 mmol) prepared in step 1 and hydrochloric acid (4.00 M solution in dioxane, 2.729 mL, 10.917 mmol) were dissolved in dichloromethane (5 mL)/methanol (5 mL) at room temperature, after which the resulting solution was stirred at the same temperature for 18 hours. Solvent was removed from the reaction mixture under reduced pressure, after which an obtained product was used without an additional purification process (title compound, 0.250 g, 86.0%, brown oil).

### [Step 3] Synthesis of (2-(7-amino-2-(furan-2-yl)-[1,2,4]triazolo[1,5-a][1,3,5]triazin-5-yl)pyrazolidin-1-yl)(4-(2,2,2-trifluoroethyl)piperazin-1-yl)methanone

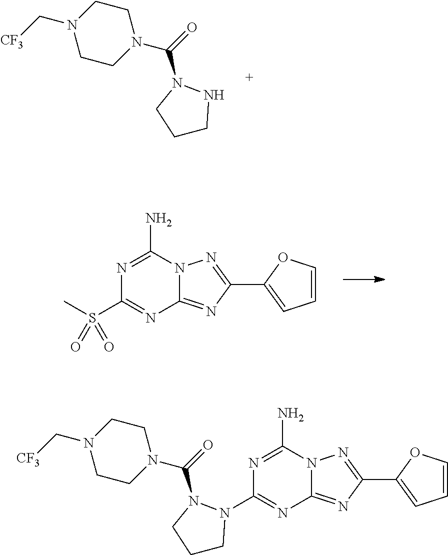

The pyrazolidin-1-yl(4-(2,2,2-trifluoroethyl)piperazin-1-yl)methanone (0.200 g, 0.751 mmol) prepared in step 2, 2-(furan-2-yl)-5-(methylsulfonyl)-[1,2,4]triazolo[1,5-a][1,3,5]triazin-7-amine (0.105 g, 0.376 mmol) and sodium hydrogen carbonate (0.189 g, 2.253 mmol) were dissolved in acetonitrile (10 mL) at room temperature, after which the resulting solution was stirred at 70° C. for 18 hours to complete the reaction by lowering a temperature to room temperature. The reaction mixture was purified via column chromatography (SiO₂, 12 g cartridge; methanol/ethyl acetate=0 to 10%) and concentrated to obtain a product, after which the obtained product was purified again via chromatography (SiO₂ plate, 20×20×1 mm; methanol/dichloromethane=10%) and concentrated to obtain a title compound (0.020 g, 5.7%) as a white solid form.

¹H NMR (400 MHz, DMSO-d₆) δ 8.40 (m, 2H), 7.88 (dd, J=1.7, 0.8 Hz, 1H), 7.08 (dd, J=3.4, 0.7 Hz, 1H), 6.69 (dd, J=3-4, 1.8 Hz, 1H), 3.73 (t, J=7.1 Hz, 2H), 3.43 (m, 6H), 3.30-3.10 (m, 2H), 2.73-2.58 (m, 4H), 2.12-1.94 (m, 2H); LRMS (ES) m/z 467.19 (M⁺+1).

### Example 272: Synthesis of corm pound 272, (2-(7-amino-2-(furan-2-yl)-[1,2,4]triazolo[1,5-a][1,3,5]triazin-5-yl)pyrazolidin-1-yl)(4-(2,2,2-trifluoroethyl)piperazin-1-yl)methanone

### [Step 1] Synthesis of tert-butyl (S)-(1-(4-(2,4-difluorophenyl)piperazin-1-yl)-3-hydroxy-1-oxopropan-2-yl)carbamate

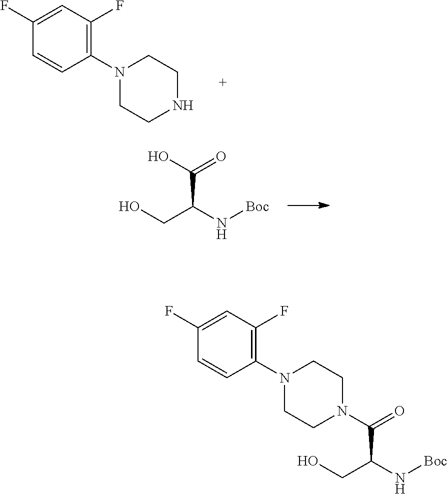

1-(2,4-difluorophenyl)piperazine (10.000 g, 50.449 mmol), (tert-butoxycarbonyl)-L-serine (20.705 g, 100.898 mmol), 1-[bis(dimethylamino)methylene]-1H-1,2,3-triazolo[4,5-b]pyridinium 3-oxide hexafluorophosphate (HATU, 38.364 g, 100.898 mmol) and N,N-diisopropylethylamine (43.936 mL, 252.245 mmol) were dissolved in dichloromethane (500 mL) at room temperature, after which the resulting solution was stirred at the same temperature for 18 hours. Solvent was removed from the reaction mixture under reduced pressure, after which saturated aqueous solution of ammonium chloride was poured into the resulting concentrate and an organic layer was extracted with ethyl acetate. The organic layer was washed with saturated aqueous solution of sodium hydrogen carbonate, dehydrated with anhydrous magnesium sulfate, filtered, and concentrated under reduced pressure. The resulting concentrate was purified via column chromatography (SiO₂, 120 g cartridge; ethyl acetate/hexane=0 to 45%) and concentrated to obtain a title compound (18.000 g, 92.6%) as a white solid of a foam type.

### [Step 2] Synthesis of (S)-2-((tert-butoxycarbonyl)amino)-3-(4-(2,4-difluorophenyl)piperazin-1-yl)-3-oxopropyl methanesulfonate

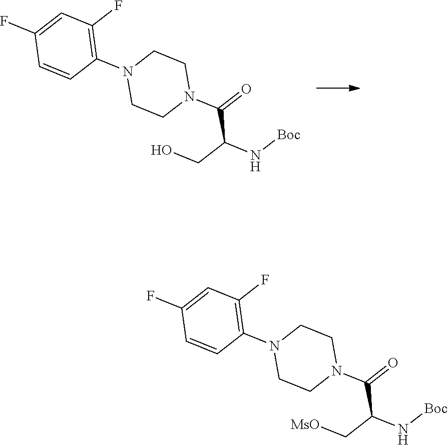 [SMILES: CC(C)(C)OC(=O)N[C@@H](CO)C(=O)N1CCN(c2ccc(F)cc2F)CC1 ; CC(C)(C)OC(=O)N[C@@H](COS(C)(=O)=O)C(=O)N1CCN(c2ccc(F)cc2F)CC1]

Tert-butyl (S)-(1-(4-(2,4-difluorophenyl)piperazin-1-yl)-3-hydroxy-1-oxopropan-2-yl)carbamate (18.000 g, 46.704 mmol) prepared in step 1, methanesulfonyl chloride (3.976 mL, 51.374 mmol) and triethylamine (13.019 mL, 93.407 mmol) were dissolved in dichloromethane (500 mL) at room temperature, after which the resulting solution was stirred at the same temperature for 18 hours. Saturated aqueous solution of ammonium chloride was poured into the reaction mixture, and an organic layer was extracted with dichloromethane. The organic layer was washed with saturated aqueous solution of sodium hydrogen carbonate, dehydrated with anhydrous magnesium sulfate, filtered, and concentrated under reduced pressure. An obtained product was used without an additional purification process (title compound, 20.000 g, 92.4%, yellow oil).

### [Step 3] Synthesis of tert-butyl (S)-(1-(4-(2,4-difluorophenyl)piperazin-1-yl)-1-oxo-3-(piperidin-1-yl)propan-2-yl)carbamate

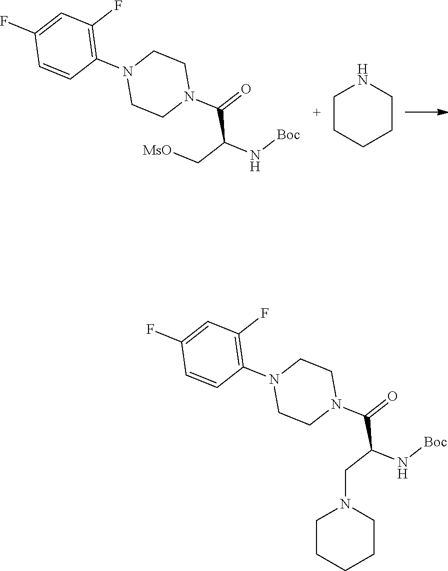 [SMILES: CC(C)(C)OC(=O)N[C@@H](COS(C)(=O)=O)C(=O)N1CCN(c2ccc(F)cc2F)CC1 ; C1CCNCC1 ; CC(C)(C)OC(=O)N[C@@H](CN1CCCCC1)C(=O)N1CCN(c2ccc(F)cc2F)CC1]

(S)-2-((Tert-butoxycarbonyl)amino)-3-(4-(2,4-difluorophenyl)piperazin-1-yl)-3-oxopropyl methanesulfonate (1.000 g, 2.157 mmol) prepared in step 2, cesium carbonate (1.406 g, 4.315 mmol) and piperidine (0.426 mL, 4.315 mmol) were dissolved in acetonitrile (10 mL) at room temperature, after which the resulting solution was stirred at 90° C. for 18 hours to complete the reaction by lowering a temperature to room temperature. Saturated aqueous solution of sodium hydrogen carbonate was poured into the reaction mixture, and an organic layer was extracted with ethyl acetate. The organic layer was washed with saturated aqueous solution of sodium chloride, dehydrated with anhydrous magnesium sulfate, filtered, and concentrated under reduced pressure. The resulting concentrate was purified via column chromatography (SiO₂, 12 g cartridge; ethyl acetate/hexane=0 to 40%) and concentrated to obtain a title compound (0.450 g, 46.1%) as a colorless oil form.

### [Step 4] Synthesis of (S)-2-amino-1-(4-(2,4-difluorophenyl)piperazin-1-yl)-3-(piperidin-1-yl)propan-1-one

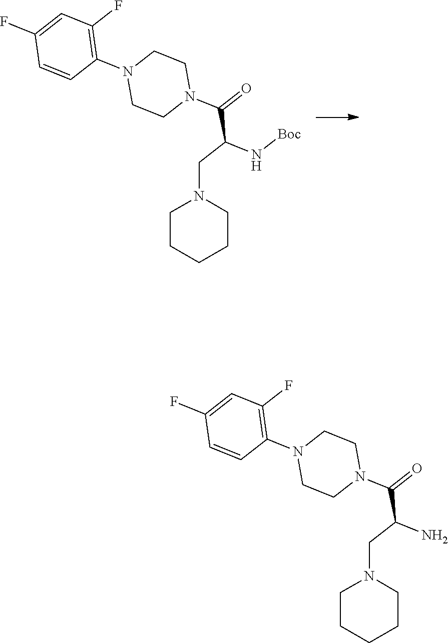 [SMILES: CC(C)(C)OC(=O)N[C@@H](CN1CCCCC1)C(=O)N1CCN(c2ccc(F)cc2F)CC1 ; N[C@@H](CN1CCCCC1)C(=O)N1CCN(c2ccc(F)cc2F)CC1]

Tert-butyl (S)-(1-(4-(2,4-difluorophenyl)piperazin-1-yl)-1-oxo-3-(piperidin-1-yl)propan-2-1)carbamate (0.200 g, 0.442 mmol) prepared in step 3 and hydrochloric acid (4.00 M solution in dioxane, 1.105 mL, 4.419 mmol) were dissolved in dichloromethane (5 mL) at room temperature, after which the resulting solution was stirred at the same temperature for 18 hours. Solvent was removed from the reaction mixture under reduced pressure, after which an obtained product was used without an additional purification process (title compound, 0.100 g, 64.2%, brown oil).

### [Step 5] Synthesis of (S)-2-((7-amino-2-(furan-2-yl)-[1,2,4]triazolo[1,5-a][1,3,5]triazin-5-yl)amino)-1-(4-(2,4-difluorophenyl)piperazin-1-yl)-3-(piperidin-1-yl)propan-1-one

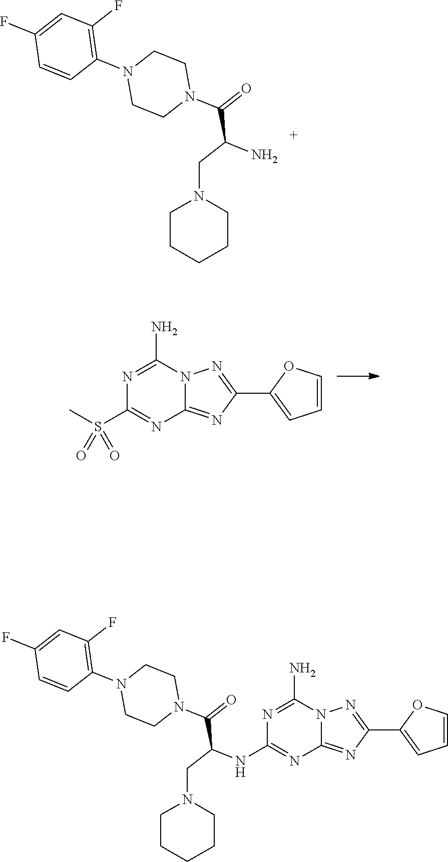 [SMILES: N[C@@H](CN1CCCCC1)C(=O)N1CCN(c2ccc(F)cc2F)CC1 ; CS(=O)(=O)c1nc(N)n2nc(-c3ccco3)nc2n1 ; Nc1nc(N[C@@H](CN2CCCCC2)C(=O)N2CCN(c3ccc(F)cc3F)CC2)nc2nc(-c3ccco3)nn12]

(S)-2-Amino-1-(4-(2,4-difluorophenyl)piperazin-1-yl)-3-(piperidin-1-yl)propan-1-one (0.100 g, 0.284 mmol) prepared in step 4, 2-(furan-2-yl)-5-(methylsulfonyl)-[1,2,4]triazolo[1,5-a][1,3,5]triazin-7-amine (0.080 g, 0.284 mmol) and sodium hydrogen carbonate (0.048 g, 0.567 mmol) were dissolved in acetonitrile (5 mL) at room temperature, after which the resulting solution was stirred at 70° C. for 18 hours to complete the reaction by lowering a temperature to room temperature. Solvent was removed from the reaction mixture under reduced pressure, after which the resulting concentrate was purified via chromatography (SiO₂ plate, 20×20×1 mm; methanol/ethyl acetate=0 to 10%) and concentrated to obtain a title compound (0.020 g, 12.8%) as a yellow solid form.

¹H NMR (400 MHz, DMSO-d₆) δ 8.31 (m, 2H), 7.87 (s, 1H), 7.45 (dd, J=37.2, 7.0 Hz, 1H), 7.26-6.89 (m, 4H), 6.68 (dd, J=3.3, 1.8 Hz, 1H), 5.12-4.95 (m, 1H), 3.65 (m, 5H), 3.22 (m, 1H), 3.08-2.78 (m, 4H), 2.68-2.56 (m, 2H), 2.37 (m, 2H), 1.42 (m, 4H), 1.35 (m, 2H).

### Example 273: Synthesis of Compound 273

Example compound 273 was synthesized through substantially the same synthesis method as a synthesis method of example compound 272 by using the (S)-2-((tert-butoxycarbonyl)amino)-3-(4-(2,4-difluorophenyl)piperazin-1-yl)-3-oxopropyl methanesulfonate prepared in step 2 except for using morpholine instead of piperidine.

¹H NMR (400 MHz, DMSO-d₆) δ 8.47-8.00 (m, 2H), 7.87 (s, 1H), 7.50 (dd, J=32.6, 7.7 Hz, 1H), 7.29-6.92 (m, 4H), 6.68 (dd, J=3.2, 1.8 Hz, 1H), 5.11-4.95 (m, 1H), 3.95-3.58 (m, 4H), 3.55 (d, J=13.7 Hz, 4H), 3.30-3.13 (m, 2H), 3.11-2.79 (m, 4H), 2.70-2.48 (m, 4H).

### Example 44: Synthesis of Compound 44, ((S)-1-(7-amino-2-(furan-2-yl)-[1,2,4]triazolo[1,5-a][1,3,5]triazin-5-yl)pyrrolidin-2-yl)((1S,4S)-5-(2,4-difluorobenzyl)-2,5-diazabicyclo[2.2.1]heptan-2-yl)methanone

### [Step 1] Synthesis of tert-butyl (1S,4S)-5-(2,4-difluorobenzyl)-2,5-diazabicyclo[2.2.1]heptan-2-carboxylate

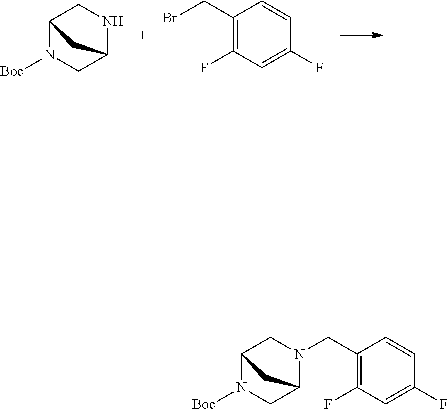 [SMILES: CC(C)(C)OC(=O)N1C[C@@H]2C[C@H]1CN2 ; Fc1ccc(CBr)c(F)c1 ; CC(C)(C)OC(=O)N1C[C@@H]2C[C@H]1CN2Cc1ccc(F)cc1F]

Tert-butyl (1S,4S)-2,5-diazabicyclo[2.2.1]heptan-2-carboxylate (10.000 g, 50.436 mmol), 1-(bromomethyl)-2,4-difluorobenzene (6.406 mL, 50.436 mmol) and potassium carbonate (27.882 g, 201.745 mmol) were dissolved in N,N-dimethylformamide (80 mL) at room temperature, after which the resulting solution was stirred at the same temperature for 18 hours. Water was poured into the reaction mixture and an organic layer was extracted with ethyl acetate. The organic layer was washed with saturated aqueous solution of sodium chloride, dehydrated with anhydrous sodium sulfate, filtered, and concentrated under reduced pressure. The resulting concentrate was purified via column chromatography (SiO₂, 40 g cartridge; ethyl acetate/hexane=0 to 50%) and concentrated to obtain a title compound (16.000 g, 97.8%) as a white solid form.

### [Step 2] Synthesis of (1S,4S)-2-(2,4-difluorobenzyl)-2,5-diazabicyclo[2.2.1]heptane

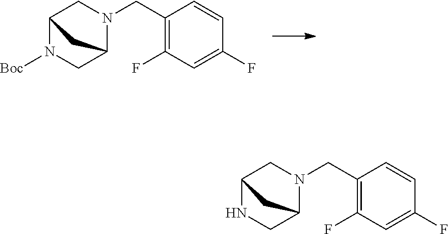 [SMILES: CC(C)(C)OC(=O)N1C[C@@H]2C[C@H]1CN2Cc1ccc(F)cc1F ; Fc1ccc(CN2C[C@@H]3C[C@H]2CN3)c(F)c1]

Tert-butyl (1S,4S)-5-(2,4-difluorobenzyl)-2,5-diazabicyclo[2.2.1]heptan-2-carboxylate (12.000 g, 36.995 mmol) prepared in step 1 and hydrochloric acid (4.00 M solution in dioxane, 92.487 mL, 369.948 mmol) were dissolved in dichloromethane (100 mL) at room temperature, after which the resulting solution was stirred at the same temperature for 18 hours. Solvent was removed from the reaction mixture under reduced pressure, after which an obtained product was used without an additional purification process (title compound, 8.000 g, 96.4%, white solid).

### [Step 3] Synthesis of tert-butyl (S)-2-((1S,4S)-5-(2,4-difluorobenzyl)-2,5-diazabicyclo[2.2.1]heptan-2-carbonyl)pyrrolidin-1-carboxylate

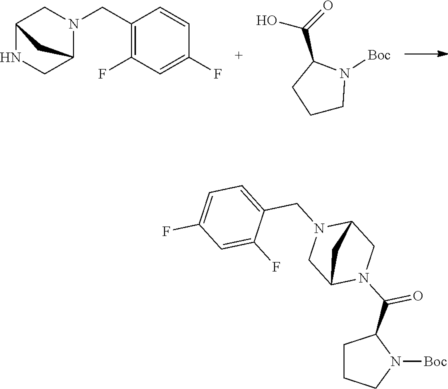 [SMILES: Fc1ccc(CN2C[C@@H]3C[C@H]2CN3)c(F)c1 ; CC(C)(C)OC(=O)N1CCC[C@H]1C(=O)O ; CC(C)(C)OC(=O)N1CCC[C@H]1C(=O)N1C[C@@H]2C[C@H]1CN2Cc1ccc(F)cc1F]

(1S,4S)-2-(2,4-Difluorobenzyl)-2,5-diazabicyclo[2.2.1]heptane (0.500 g, 2.230 mmol) prepared in step 2, (tert-butoxycarbonyl)-L-proline (0.960 g, 4.459 mmol), [dimethylamino(triazolo[4,5-b]pyridin-3-yloxy)methylidene]-dimethylazanium; hexafluorophosphate (1.696 g, 4.459 mmol) and N,N-diisopropylethylamine (0.777 mL, 4.459 mmol) were dissolved in N,N-dimethylformamide (15 mL) at room temperature, and the resulting solution was stirred at the same temperature for 18 hours. Water was poured into the reaction mixture and an organic layer was extracted with ethyl acetate. The organic layer was washed with saturated aqueous solution of sodium chloride, dehydrated with anhydrous magnesium sulfate, filtered, and concentrated under reduced pressure. The resulting concentrate was purified via column chromatography (SiO₂, 12 g cartridge; methanol/dichloromethane=0 to 10%) and concentrated to obtain a title compound (0.200 g, 21.3%) as a white solid form.

### [Step 4] Synthesis of (1S,4S)-2-(L-prolyl)-5-(2,4-difluorobenzyl)-2,5-diazabicyclo[2.2.1]heptane

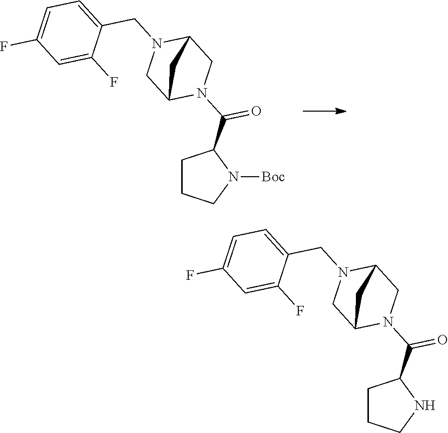

Tert-butyl (S)-2-((1S,4S)-5-(2,4-difluorobenzyl)-2,5-diazabicyclo[2.2.1]heptan-2-carbonyl)pyrrolidin-1-carboxylate (0.200 g, 0.475 mmol) prepared in step 3 and hydrochloric acid (4.00 M solution in dioxane, 1.186 mL, 4.745 mmol) were dissolved in dichloromethane (5 mL) at room temperature, after which the resulting solution was stirred at the same temperature for 18 hours. Solvent was removed from the reaction mixture under reduced pressure, after which an obtained product was used without an additional purification process (title compound, 0.150 g, 98.4%, white solid).

### [Step 5] Synthesis of ((S)-1-(7-amino-2-(furan-2-yl)-[1,2,4]triazolo[1,5-a][1,3,5]triazin-5-yl)pyrrolidin-2-yl)((1S,4S)-5-(2,4-difluorobenzyl)-2,5-diazabicyclo[2.2.1]heptan-2-yl)methanone

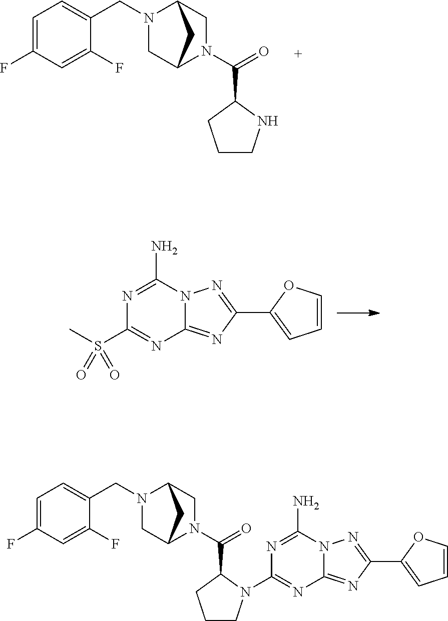 [SMILES: O=C([C@@H]1CCCN1)N1C[C@@H]2C[C@H]1CN2Cc1ccc(F)cc1F ; CS(=O)(=O)c1nc(N)n2nc(-c3ccco3)nc2n1 ; Nc1nc(N2CCC[C@H]2C(=O)N2C[C@@H]3C[C@H]2CN3Cc2ccc(F)cc2F)nc2nc(-c3ccco3)nn12]

(1S,4S)-2-(L-Prolyl)-5-(2,4-difluorobenzyl)-2,5-diazabicyclo[2.2.1]heptane (0.200 g, 0.622 mmol) prepared in step 4, 2-(furan-2-yl)-5-(methylsulfonyl)-[1,2,4]triazolo[1,5-a][1,3,5]triazin-7-amine (0.087 g, 0.311 mmol) and triethylamine (0.173 mL, 1.245 mmol) were dissolved in dimethylsulfoxide (5 mL) at room temperature, after which the resulting solution was stirred at the same temperature for 18 hours. Water was poured into the reaction mixture and an organic layer was extracted with ethyl acetate. The organic layer was washed with saturated aqueous solution of sodium chloride, dehydrated with anhydrous magnesium sulfate, filtered, and concentrated under reduced pressure. The resulting concentrate was purified via chromatography (SiO₂ plate, 20×20×1 mm; methanol/dichloromethane=10%) and concentrated to obtain a title compound (0.040 g, 12.3%) as a white solid form.

¹H NMR (400 MHz, DMSO-d₆) δ 8.33 (m, 1H), 7.90-7.44 (m, 2H), 7.18 (m, 1H), 7.12-6.87 (m, 2H), 6.67 (m, 1H), 4.66 (m, 2H), 4.13-3.37 (m, 7H), 3.29-2.74 (m, 2H), 2.47-2.20 (m, 2H), 2.14-1.64 (m, 5H); LRMS (ES) m/z 522.4 (M⁺+1).

### Example 49, 50, 51 and 52

Example compounds 49, 50, 51 and 52 were each synthesized through substantially the same synthesis method as a synthesis method of example compound 44 except for using the compounds of the following table instead of tert-butyl (1S,4S)-2,5-diazabicyclo[2.2.1]heptan-2-carboxylate of step 1.

TABLE 21

| Example | |
|---|---|
| No. | Starting Materials |
| 49 | 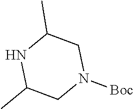 |
| 50 | 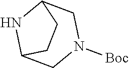 [SMILES: CC(C)(C)OC(=O)N1CC2CCC(C1)N2] |
| 51 | 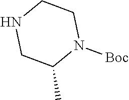 |
| 52 | 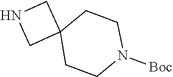 [SMILES: CC(C)(C)OC(=O)N1CCC2(CC1)CNC2] |

### Examples 30 and 31

Example compounds 30 and 31 were each synthesized through substantially the same synthesis method as a synthesis method of example compound 44 except for using the compounds of the following table instead of tert-butyl (1S,4S)-2,5-diazabicyclo[2.2.1]heptan-2-carboxylate of step 1 and using (tert-butoxycarbonyl)-L-alanine instead of (tert-butoxycarbonyl)-L-proline of step 3.

TABLE 22

| Example | |
|---|---|
| No. | Starting Material |
| 30 | 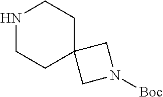 [SMILES: CC(C)(C)OC(=O)N1CC2(CCNCC2)C1] |
| 31 | 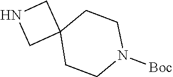 [SMILES: CC(C)(C)OC(=O)N1CCC2(CC1)CNC2] |

### Example 32: Synthesis of Compound 32

Example compound 32 was synthesized through substantially the same synthesis method as a synthesis method of example compound 44 except for using (tert-butoxycarbonyl)-L-alanine instead of (tert-butoxycarbonyl)-L-proline of step 3.

Analysis data of each of the compounds prepared as described above are shown in the table below.

TABLE 23

| Example | | |
|---|---|---|
| No. | Compound Names | Analysis Data |
| 30 | (S)-2-((7-amino-2-(furan-2-yl)- | ¹H NMR (400 MHz, DMSO-d₆) δ 8.53-8.05 (m, |
| | [1,2,4]triazolo[1,5- | 2H), 7.87 (d, J = 1.8 Hz, 1H), 7.53 (d, J = 7.1 Hz, |
| | a][1,3,5]triazin-5-yl)amino)-1- | 1H), 7.45 (q, J = 8.1 Hz, 1H), 7.25-7.16 (m, 1H), |
| | (7-(2,4-difluorobenzyl)-2,7- | 7.07 (dt, J = 10.4, 5.7 Hz, 1H), 6.99 (s, 1H), 6.67 (s, |
| | diazaspiro[3.5]nonan-2- | 1H), 4.52-4.34 (m, 1H), 4.26-4.06 (m, 1H), 3.90-3.77 |
| | yl)propan-1-one | (m, 1H), 3.59-3.40 (m, 4H), 2.30 (m, 4H), |
| | | 2.01 (s, 0H), 1.87-1.58 (m, 4H), 1.26 (d, J = 7.0 |
| | | Hz, 3H); LRMS (ES) m/z 424.4 (M⁺ + 1). |
| 31 | (S)-2-((7-amino-2-(furan-2-yl)- | ¹H NMR (400 MHz, DMSO-d₆) δ 8.24 (d, J = 57.6 |
| | [1,2,4]triazolo[1,5- | Hz, 2H), 7.90-7.85 (m, 1H), 7.50-7.34 (m, 2H), |
| | a][1,3,5]triazin-5-yl)amino)-1- | 7.20 (td, J = 9.7, 2.4 Hz, 1H), 7.10-7.00 (m, 2H), |
| | (2-(2,4-difluorobenzyl)-2,7- | 6.68 (dd, J = 3.4, 1.8 Hz, 1H), 4.92-4.82 (m, 1H), |
| | diazaspiro[3.5]nonan-7- | 3.77-3.38 (m, 6H), 3.17-2.85 (m, 4H), 1.86 (d, J = |
| | yl)propan-1-one | 9.6 Hz, 1H), 1.75-1.58 (m, 3H), 1.29-1.21 (m, |
| | | 3H); LRMS (ES) m/z 424.4 (M⁺ + 1). |
| 32 | (S)-2-((7-amino-2-(furan-2-yl)- | ¹H NMR (400 MHz, DMSO-d₆) δ 8.24 (d, J = 60.5 |
| | [1,2,4]triazolo[1,5- | Hz, 2H), 7.90-7.84 (m, 1H), 7.66 (dd, J = 17.4, 8.0 |
| | a][1,3,5]triazin-5-yl)amino)-1- | Hz, 1H), 7.51 (d, J = 6.2 Hz, 1H), 7.16 (dd, J = 16.5, |
| | ((1S, 4S)-5-(2,4-difluorobenzyl)- | 7.2 Hz, 1H), 7.10-6.98 (m, 2H), 6.68 (dt, J = 3.4, |
| | 2,5-diazabicyclo[2.2.1]heptan- | 1.7 Hz, 1H), 4.70-4.54 (m, 1H), 4.53-4.41 (m, |
| | 2-yl)propan-1-one | 1H), 3.86-3.59 (m, 3H), 3.55-3.48 (m, 1H), 3.17-3.05 |
| | | (m, 1H), 2.89-2.78 (m, 1H), 2.61-2.53 (m, |
| | | 2H), 1.93-1.56 (m, 2H), 1.37-1.21 (m, 3H); LRMS |
| | | (ES) m/z 496.4 (M⁺ + 1). |
| 49 | ((S)-1-(7-amino-2-(furan-2-yl)- | ¹H NMR (400 MHz, DMSO-d₆) δ 8.61-8.03 (m, |
| | [1,2,4]triazolo[1,5- | 2H), 7.88 (s, 1H), 7.78-7.64 (m, 1H), 7.17 (t, J = |
| | a][1,3,5]triazin-5-yl)pyrrolidin- | 10.0 Hz, 1H), 7.13-6.93 (m, 2H), 6.78-6.59 (m, |
| | 2-yl)(4-(2,4-difluorobenzyl)- | 1H), 5.17-4.89 (m, 1H), 4.26-3.46 (m, 6H), 3.22-2.83 |
| | 3,5-dimethylpiperazin-1- | (m, 2H), 2.71-2.55 (m, 2H), 2.41-2.16 (m, |
| | yl)methanone | 1H), 2.02-1.76 (m, 3H), 1.09-0.78 (m, 6H); |
| | | LRMS (ES) m/z 538.4 (M⁺ + 1). |
| 50 | ((S)-1-(7-amino-2-(furan-2-yl)- | ¹H NMR (400 MHz, DMSO-d₆) δ 8.60-8.01 (m, |
| | [1,2,4]triazolo[1,5- | 2H), 7.87 (s, 1H), 7.71-7.51 (m, 1H), 7.26-7.15 |
| | a][1,3,5]triazin-5-yl)pyrrolidin- | (m, 1H), 7.14-7.02 (m, 2H), 6.71-6.64 (m, 1H), |
| | 2-yl)(8-(2,4-difluorobenzyl)- | 5.11-4.76 (m, 1H), 4.14-3.79 (m, 1H), 3.79-3.39 |
| | 3,8-diazabicyclo[3.2.1]octan-3- | (m, 5H), 3.30-3.09 (m, 3H), 2.96-2.62 (m, 1H), |
| | yl)methanone | 2.43-2.11 (m, 1H), 2.09-1.72 (m, 5H), 1.71-1.35 |
| | | (m, 2H); LRMS (ES) m/z 536.4 (M⁺ + 1). |
| 51 | ((S)-1-(7-amino-2-(furan-2-yl)- | ¹H NMR (400 MHz, DMSO-d₆) δ 8.56-7.97 (m, |
| | [1,2,4]triazolo[1,5- | 2H), 7.95-7.79 (m, 1H), 7.60-7.37 (m, 1H), 7.29-7.16 |
| | a][1,3,5]triazin-5-yl)pyrrolidin- | (m, 1H), 7.15-6.99 (m, 2H), 6.78-6.58 (m, |
| | 2-yl)((R)-4-(2,4- | 1H), 5.13-4.81 (m, 1H), 4.46 (s, 1H), 4.30-3.98 |
| | difluorobenzyl)-2- | (m, 1H), 3.74-3.39 (m, 4H), 3.00-2.58 (m, 2H), |
| | methylpiperazin-1- | 2.39-2.11 (m, 2H), 2.11-1.66 (m, 4H), 1.48 (dd, J = |
| | yl)methanone | 18.6, 6.5 Hz, 1H), 1.40-1.21 (m, 1H), 1.14 (dd, J = |
| | | 13.4, 6.7 Hz, 2H); LRMS (ES) m/z 524.6 (M⁺ + 1). |
| 52 | (S)-(1-(7-amino-2-(furan-2-yl)- | ¹H NMR (400 MHz, DMSO-d₆) δ 8.57-8.09 (m, |
| | [1,2,4]triazolo[1,5- | 2H), 7.91-7.81 (m, 1H), 7.50-7.39 (m, 1H), 7.25-7.15 |
| | a][1,3,5]triazin-5-yl)pyrrolidin- | (m, 1H), 7.13-6.90 (m, 2H), 6.71-6.63 (m, |
| | 2-yl)(2-(2,4-difluorobenzyl)- | 1H), 4.60-4.41 (m, 1H), 4.41-4.08 (m, 1H), 3.82 |
| | 2,7-diazaspiro[3.5]nonan-7- | (dd, J = 35.5, 8.1 Hz, 1H), 3.71-3.40 (m, 6H), |
| | yl)methanone | 2.48-2.11 (m, 5H), 2.09-1.82 (m, 3H), 1.82-1.62 |
| | | (m, 4H); LRMS (ES) m/z 550.4 (M⁺ + 1). |

### Example 157: Synthesis of Compound 157, (3-((7-amino-2-(furan-2-yl)-[1,2,4]triazolo[1,5-a][1,3,5]triazin-5-yl)-L-prolyl)-3,8-diazabicyclo[3.2.1]octan-8-yl)(phenyl)methanone

### [Step 1] Synthesis of tert-butyl 8-benzoyl-3,8-diazabicyclo[3.2.1]octan-3-carboxylate

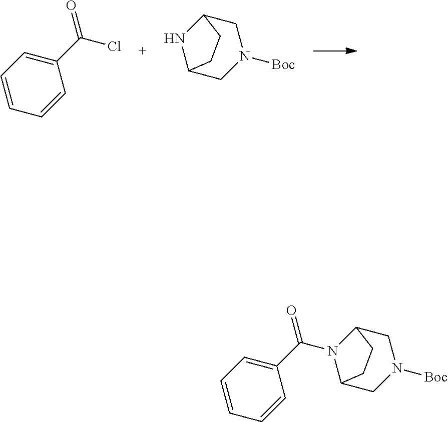 [SMILES: O=C(Cl)c1ccccc1 ; CC(C)(C)OC(=O)N1CC2CCC(C1)N2 ; CC(C)(C)OC(=O)N1CC2CCC(C1)N2C(=O)c1ccccc1]

Tert-butyl 3,8-diazabicyclo[3.2.1]octan-3-carboxylate (0.319 g, 1.503 mmol), benzoyl chloride (0.175 mL, 1.503 mmol) and triethylamine (0.314 mL, 2.254 mmol) were dissolved in dichloromethane (5 mL) at room temperature, after which the resulting solution was stirred at the same temperature for 18 hours. Aqueous solution of N-sodium hydrogen carbonate was poured into the reaction mixture, and then an organic layer was extracted with dichloromethane, filtered via a plastic filter to remove a solid residue and an aqueous solution layer therefrom, and concentrated under reduced pressure. An obtained product was used without an additional purification process (title compound, 0.474 g, 99.7%, light yellow oil).

### [Step 2] Synthesis of (3,8-diazabicyclo[3.2.1]octan-8-yl)(phenyl)methanone hydrochloride

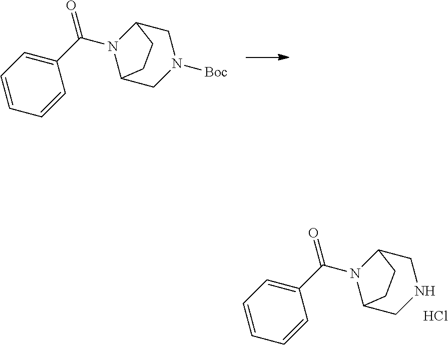 [SMILES: CC(C)(C)OC(=O)N1CC2CCC(C1)N2C(=O)c1ccccc1 ; O=C(c1ccccc1)N1C2CCC1CNC2 ; Cl]

Tert-butyl 8-benzoyl-3,8-diazabicyclo[3.2.1]octan-3-carboxylate (0.474 g, 1.498 mmol) prepared in step 1 and hydrochloric acid (4.00 M solution in 1,4-dioxane, 1.498 mL, 5.992 mmol) were dissolved in dichloromethane (5 mL) at room temperature, after which the resulting solution was stirred at the same temperature for 18 hours. Solvent was removed from the reaction mixture under reduced pressure, after which an obtained product was used without an additional purification process (title compound, 0.378 g, 99.8%, light yellow oil).

### [Step 3] Synthesis of tert-butyl (2S)-2-(8-benzoyl-3,8-diazabicyclo[3.2.1]octan-3-carbonyl)pyrrolidin-1-carboxylate

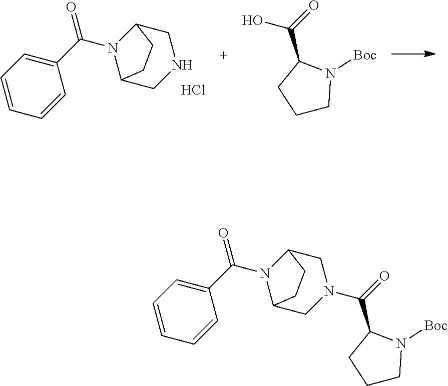 [SMILES: O=C(c1ccccc1)N1C2CCC1CNC2 ; Cl ; CC(C)(C)OC(=O)N1CCC[C@H]1C(=O)O ; CC(C)(C)OC(=O)N1CCC[C@H]1C(=O)N1CC2CCC(C1)N2C(=O)c1ccccc1]

(3,8-Diazabicyclo[3.2.1]octan-8-yl)(phenyl)methanone hydrochloride (0.378 g, 1.496 mmol) prepared in step 2, (tert-butoxycarbonyl)-L-proline (0.322 g, 1.496 mmol), 2,4,6-tripropyl-1,3,5,2,4,6-trioxatriphosphineine 2,4,6-trioxide (T3P, 50.00% solution in EtOAc, 1.334 mL, 2.243 mmol) and N,N-diisopropylethylamine (0.782 mL, 4.487 mmol) were dissolved in dichloromethane (8 mL) at room temperature, after which the resulting solution was stirred at the same temperature for 18 hours. Aqueous solution of N-sodium hydrogen carbonate was poured into the reaction mixture, and then an organic layer was extracted with dichloromethane, filtered via a plastic filter to remove a solid residue and an aqueous solution layer therefrom, and concentrated under reduced pressure. The resulting concentrate was purified via column chromatography (SiO₂, 12 g cartridge; dichloromethane/methanol=0 to 5%) and concentrated to obtain a title compound (0.424 g, 68.6%) as a transparent oil form.

### [Step 4] Synthesis of (3-(L-prolyl)-3,8-diazabicyclo[3.2.1]octan-8-yl)(phenyl)methanone hydrochloride

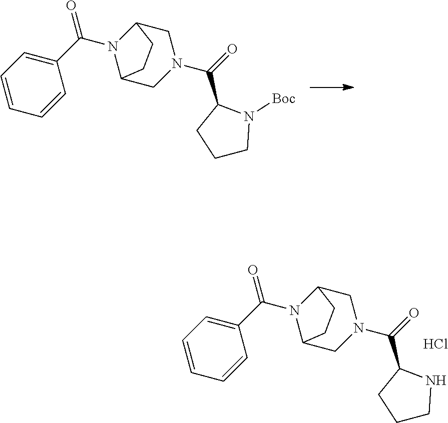 [SMILES: CC(C)(C)OC(=O)N1CCC[C@H]1C(=O)N1CC2CCC(C1)N2C(=O)c1ccccc1 ; O=C([C@@H]1CCCN1)N1CC2CCC(C1)N2C(=O)c1ccccc1 ; Cl]

Tert-butyl (2S)-2-(8-benzoyl-3,8-diazabicyclo[3.2.1]octan-3-carbonyl)pyrrolidin-1-carboxylate (0.424 g, 1.025 mmol) prepared in step 3 and hydrogen chloride (4.00 M solution in 1,4-dioxane, 1.025 mL, 4.101 mmol) were dissolved in dichloromethane (5 mL) at room temperature, after which the resulting solution was stirred at the same temperature for 18 hours. Solvent was removed from the reaction mixture under reduced pressure, after which an obtained product was used without an additional purification process (title compound, 0.358 g, 99.8%, light yellow oil).

### [Step 5] Synthesis of (3-((7-amino-2-(furan-2-yl)-[1,2,4]triazolo[1,5-a][1,3,5]triazin-5-yl)-L-prolyl)-3,8-diazabicyclo[3.2.1]octan-8-yl)(phenyl)methanone

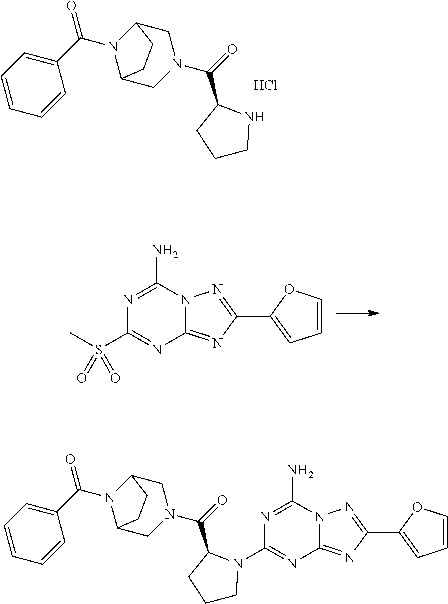 [SMILES: O=C([C@@H]1CCCN1)N1CC2CCC(C1)N2C(=O)c1ccccc1 ; Cl ; CS(=O)(=O)c1nc(N)n2nc(-c3ccco3)nc2n1 ; Nc1nc(N2CCC[C@H]2C(=O)N2CC3CCC(C2)N3C(=O)c2ccccc2)nc2nc(-c3ccco3)nn12]

(3-(L-Prolyl)-3,8-diazabicyclo[3.2.1]octan-8-yl)(phenyl)methanone hydrochloride (0.358 g, 1.023 mmol) prepared in step 4, 2-(furan-2-yl)-5-(methylsulfonyl)-[1,2,4]triazolo[1,5-a][1,3,5]triazin-7-amine (0.287 g, 1.023 mmol) and sodium hydrogen carbonate (0.258 g, 3.070 mmol) were dissolved in cyclopentylmethyl ether (CPME, 5 mL) at room temperature, after which the resulting solution was stirred at 50° C. for 18 hours to complete the reaction by lowering a temperature to room temperature. Solvent was removed from the reaction mixture under reduced pressure, after which the resulting concentrate was purified via column chromatography (SiO₂, 12 g cartridge; dichloromethane/methanol=0 to 5%) and concentrated to obtain a title compound (0.309 g, 58.9%) as a white solid form.

¹H NMR (400 MHz, Chloroform-d) δ 7.69-7.33 (m, 5H), 7.24-7.09 (m, 1H), 6.51 (td, J=3-7, 1.7 Hz, 1H), 6.31 (s, 1H), 5.18-4.69 (m, 2H), 4.16 (dd, J=78.6, 54.6 Hz, 2H), 3.92-3.49 (m, 3H), 3.42-2.78 (m, 1H), 2.61 (s, 1H), 2.53-2.26 (m, 1H), 2.26-2.09 (m, 2H), 2.00 (dp, J=17.5, 7.3, 6.1 Hz, 3H), 1.92-1.83 (m, 1H), 1.74 (dd, J=15.6, 8.1 Hz, 1H). LRMS (ES) m/z 514.5 (M⁺+1).

### Examples 158, 159, 160 and 161

Example compounds 158 to 161 were each synthesized through substantially the same synthesis method as a synthesis method of example compound 157 except for using the compounds of the following table instead of tert-butyl 3,8-diazabicyclo[3.2.1]octan-3-carboxylate of step 1 as a starting material.

TABLE 24

| Example | |
|---|---|
| No. | Starting Materials |
| 158 | 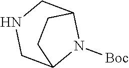 [SMILES: CC(C)(C)OC(=O)N1C2CCC1CNC2] |
| 159 | 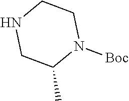 |
| 160 | 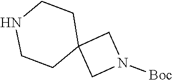 [SMILES: CC(C)(C)OC(=O)N1CC2(CCNCC2)C1] |
| 161 | 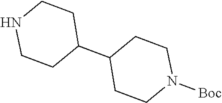 [SMILES: CC(C)(C)OC(=O)N1CCC(C2CCNCC2)CC1] |

Analysis data of each of the compounds prepared as described above are shown in the table below.

TABLE 25

| Example | | |
|---|---|---|
| No. | Compound Names | Analysis Data |
| 158 | (8-((7-amino-2-(furan-2-yl)- | ¹H NMR (400 MHz, DMSO-d₆) δ 8.21 (s, 2H), |
| | [1,2,4]triazolo[1,5-a][1,3,5]triazin- | 7.89-7.82 (m, 1H), 7.63-7.36 (m, 5H), 7.11-6.98 |
| | 5-yl)-L-prolyl)-3,8- | (m, 1H), 6.66 (ddd, J = 9.4, 3.9, 1.9 Hz, 1H), |
| | diazabicyclo[3.2.1]octan-3- | 5.15-4.60 (m, 2H), 4.05 (d, J = 21.0 Hz, 3H), |
| | yl)(phenyl)methanone | 3.64 (dqd, J = 22.3, 11.0, 4.6 Hz, 3H), 2.90 (d, J = |
| | | 13.5 Hz, 1H), 2.44-2.12 (m, 1H), 2.08-1.73 |
| | | (m, 5H), 1.62 (d, J = 49.6 Hz, 1H). LRMS (ES) |
| | | m/z 514.6 (M⁺ + 1). |
| 159 | ((R)-4-((7-amino-2-(furan-2-yl)- | ¹H NMR (400 MHz, DMSO-d₆) δ 8.15 (d, J = 12.2 |
| | [1,2,4]triazolo[1,5-a][1,3,5]triazin- | Hz, 2H), 7.87 (d, J = 2.0 Hz, 1H), 7.56-7.35 (m, |
| | 5-yl)-L-prolyl)-3-methylpiperazin- | 5H), 7.07 (q, J = 4.8, 3.8 Hz, 1H), 6.67 (td, J = |
| | 1-yl)(phenyl)methanone | 3.8, 1.8 Hz, 1H), 5.17-4.75 (m, 1H), 4.69-3.77 |
| | | (m, 2H), 3.64 (dt, J = 19.2, 7.0 Hz, 3H), 2.95 (s, |
| | | 1H), 2.30 (d, J = 28.4 Hz, 1H), 2.08-1.68 (m, |
| | | 4H), 1.35-0.90 (m, 4H). LRMS (ES) m/z 502.6 |
| | | (M⁺ + 1). |
| 160 | (2-((7-amino-2-(furan-2-yl)- | ¹H NMR (400 MHz, DMSO-d₆) δ 8.39 (d, J = |
| | [1,2,4]triazolo[1,5-a][1,3,5]triazin- | 101.0 Hz, 2H), 7.94-7.75 (m, 1H), 7.52-7.35 |
| | 5-yl)-L-prolyl)-2,7- | (m, 5H), 7.12-6.66 (m, 1H), 6.60 (s, 1H), 4.49 |
| | diazaspiro[3.5]nonan-7- | (ddd, J = 49.7, 8.6, 3.5 Hz, 2H), 4.25-3.78 (m, |
| | yl)(phenyl)methanone | 2H), 3.72-3.46 (m, 11H), 2.19 (td, J = 8.2, 4.9 |
| | | Hz, 1H), 2.03 (s, 2H), 1.89 (ddd, J = 12.8, 10.3, |
| | | 5.5 Hz, 3H), 1.73 (s, 3H). LRMS (ES) m/z 528.6 |
| | | (M⁺ + 1). |
| 161 | (1′-((7-amino-2-(furan-2-yl)- | ¹H NMR (400 MHz, DMSO-d₆) δ 8.34 (d, J = |
| | [1,2,4]triazolo[1,5-a][1,3,5]triazin- | 114.6 Hz, 2H), 7.89 (dd, J = 4.6, 1.7 Hz, 1H), |
| | 5-yl)-L-prolyl)-[4,4′-bipiperidin]- | 7.48-7.31 (m, 5H), 7.14-6.93 (m, 1H), 6.72-6.59 |
| | 1-yl)(phenyl)methanone | (m, 1H), 5.00 (td, J = 10.1, 8.7, 5.4 Hz, 1H), |
| | | 4.69-4.29 (m, 2H), 4.19-3.96 (m, 1H), 3.75-3.46 |
| | | (m, 7H), 2.98 (dd, J = 15.0, 8.1 Hz, 2H), 2.69 (s, |
| | | 1H), 2.30-2.18 (m, 1H), 1.97-1.47 (m, 8H), |
| | | 1.46-0.90 (m, 7H). LRMS (ES) m/z 570.7 |
| | | (M⁺ + 1). |

### Example 277: Synthesis of corm pound 277, (S)-(1-(7-amino-2-(furan-2-yl)-[1,2,4]triazolo[1,5-a][1,3,5]triazin-5-yl)pyrrolidin-2-yl)(4-(pyrimidin-2-yl)-1,4-diazepan-1-yl)methanone

### [Step 1] Synthesis of tert-butyl 4-(pyrimidin-2-yl)-1,4-diazepan-1-carboxylate

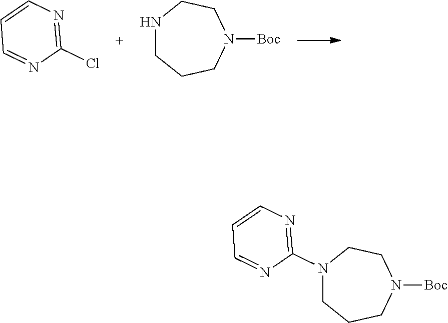 [SMILES: Clc1ncccn1 ; CC(C)(C)OC(=O)N1CCCNCC1 ; CC(C)(C)OC(=O)N1CCCN(c2ncccn2)CC1]

2-Chloropyrimidine (0.229 g, 1.999 mmol), tert-butyl 1,4-diazepan-1-carboxylate (0.400 g, 1.999 mmol) and potassium carbonate (0.829 g, 5.998 mmol) were dissolved in acetonitrile (10 mL) at room temperature, after which the resulting solution was stirred at 80° C. for 18 hours to complete the reaction by lowering a temperature to room temperature. Water was poured into the reaction mixture and an organic layer was extracted with dichloromethane. The organic layer was washed with saturated aqueous solution of sodium chloride, dehydrated with anhydrous sodium sulfate, filtered, and concentrated under reduced pressure. The resulting concentrate was purified via column chromatography (SiO₂, 12 g cartridge; dichloromethane/methanol=0 to 30%) and concentrated to obtain a title compound (0.556 g, 99.9%) as a light yellow liquid form.

### [Step 2] Synthesis of 1-(pyrimidin-2-yl)-1,4-diazepane hydrochloride

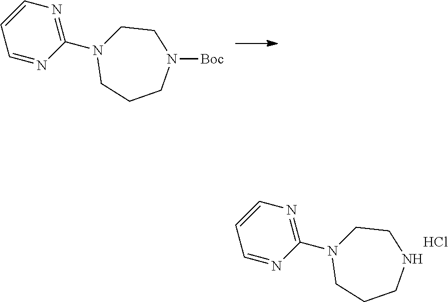 [SMILES: CC(C)(C)OC(=O)N1CCCN(c2ncccn2)CC1 ; c1cnc(N2CCCNCC2)nc1 ; Cl]

Tert-butyl 4-(pyrimidin-2-yl)-1,4-diazepain-1-carboxylate (0.556 g, 1.997 mmol) prepared in step 1 and hydrochloric acid (4.00 M solution in 1,4-dioxane, 1.997 mL, 7.990 mmol) were dissolved in dichloromethane (5 mL) at room temperature, after which the resulting solution was stirred at the same temperature for five hours. Solvent was removed from the reaction mixture under reduced pressure, after which an obtained product was used without an additional purification process (title compound, 0.215 g, 50.1%, white solid).

### [Step 3] Synthesis of tert-butyl (S)-2-(4-(pyrimidin-2-yl)-1,4-diazepan-1-carbonyl)pyrrolidin-1-carboxylate

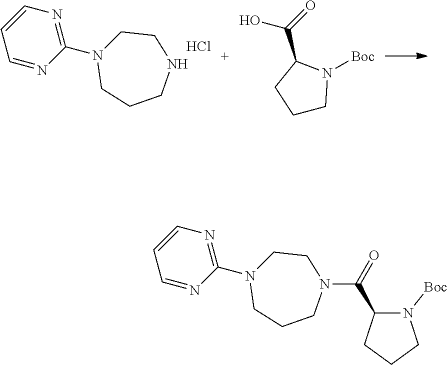 [SMILES: c1cnc(N2CCCNCC2)nc1 ; Cl ; CC(C)(C)OC(=O)N1CCC[C@H]1C(=O)O ; CC(C)(C)OC(=O)N1CCC[C@H]1C(=O)N1CCCN(c2ncccn2)CC1]

1-(Pyrimidin-2-yl)-1,4-diazepane hydrochloride (0.214 g, 0.997 mmol) prepared in step 2, (tert-butoxycarbonyl)-L-proline (0.215 g, 0.997 mmol), 2,4,6-tripropyl-1,3,5,2,4,6-trioxatriphosphinane 2,4,6-trioxide (T3P, 50.00% solution in EtOAc, 0.914 mL, 1.495 mmol) and N,N-diisopropylethylamine (0.694 mL, 3.987 mmol) were dissolved in dichloromethane (5 mL) at room temperature, after which the resulting solution was stirred at the same temperature for 18 hours. Aqueous solution of N-sodium hydrogen carbonate was poured into the reaction mixture, and then an organic layer was extracted with dichloromethane, filtered via a plastic filter to remove a solid residue and an aqueous solution layer therefrom, and concentrated under reduced pressure. An obtained product was used without an additional purification process (title compound, 0.3750 g, 100.0%, light yellow liquid).

### [Step 4] Synthesis of (S)-1-prolyl-4-(pyrimidin-2-yl)-1,4-diazepane hydrochloride

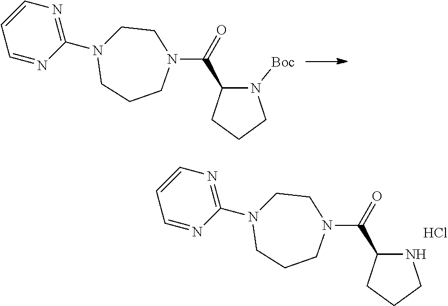

Tert-butyl (S)-2-(4-(pyrimidin-2-yl)-1,4-diazepan-1-carbonyl)pyrrolidin-1-carboxylate (0.375 g, 0.999 mmol) prepared in step 3 and hydrochloric acid (4.00 M solution in 1,4-dioxane, 0.999 mL, 3.995 mmol) were dissolved in dichloromethane (5 mL) at room temperature, after which the resulting solution was stirred at the same temperature for 18 hours. Solvent was removed from the reaction mixture under reduced pressure, after which an obtained product was used without an additional purification process (title compound, 0.311 g, 99.9%, white solid).

### [Step 5] Synthesis of (S)-(1-(7-amino-2-(furan-2-yl)-[1,2,4]triazolo[1,5-a][1,3,5]triazin-5-yl)pyrrolidin-2-yl)(4-(pyrimidin-2-yl)-1,4-diazepan-1-yl)methanone

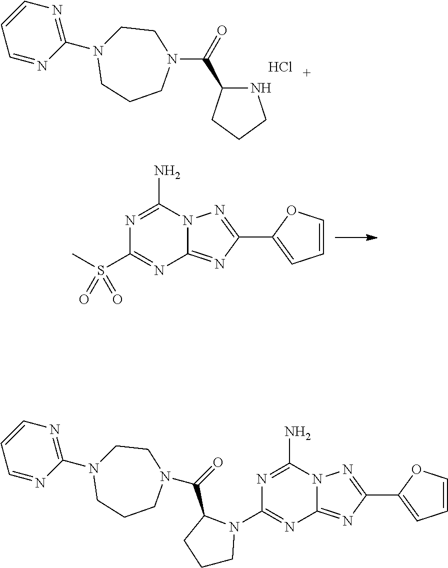 [SMILES: O=C([C@@H]1CCCN1)N1CCCN(c2ncccn2)CC1 ; Cl ; CS(=O)(=O)c1nc(N)n2nc(-c3ccco3)nc2n1 ; Nc1nc(N2CCC[C@H]2C(=O)N2CCCN(c3ncccn3)CC2)nc2nc(-c3ccco3)nn12]

(S)-1-Prolyl-4-(pyrimidin-2-yl)-1,4-diazepane hydrochloride (0.311 g, 0.997 mmol) prepared in step 4, 2-(furan-2-yl)-5-(methylsulfonyl)-[1,2,4]triazolo[1,5-a][1,3,5]triazin-7-amine (0.280 g, 0.997 mmol) and sodium hydrogen carbonate (0.251 g, 2.992 mmol) were dissolved in acetonitrile (5 mL) at room temperature, after which the resulting solution was stirred at 70° C. for 18 hours to complete the reaction by lowering a temperature to room temperature. The reaction mixture was filtered via a celite pad to remove a solid therefrom, after which solvent was removed from the resulting filtrate without the solid under reduced pressure. Then, the resulting concentrate was purified via column chromatography (SiO₂, 12 g cartridge; dichloromethane/methanol=0 to 30%) and concentrated to obtain a title compound (0.109 g, 23.0%) as a white solid form.

¹H NMR (400 MHz, DMSO-d₆) δ 7.86 (dd, J=1.7, 0.8 Hz, 1H), 7.46 (brs, 2H), 7.06 (dd, J=3.4, 0.6 Hz, 1H), 6.67 (dd, J=3.4, 1.8 Hz, 1H), 5.59 (brs, 1H), 4.96 (brs, 1H), 3.79-3.38 (m, 5H), 2.79 (t, J=14.1 Hz, 3H), 2.64-2.36 (m, 4H), 2.24 (s, 1H), 2.06-1.77 (m, 3H), 1.66 (t, J=19.1 Hz, 3H); LRMS (ES) m/z 461.5 (M⁺+1).

### Examples 328, 329 and 384

Example compounds 328, 329 and 384 were each synthesized through substantially the same synthesis method as a synthesis method of example compound 277 except for using the starting material 1 of the table below instead of 2-chloropyrimidine in step 1, using the starting material 2 of the table below instead of tert-butyl 1,4-diazepan-1-carboxylate, and using (S)-1-(tert-butoxycarbonyl)azetidin-2-carboxylic acid instead of (tert-butoxycarbonyl)-L-proline in step 3.

TABLE 26

| Example | Starting material 1 | |
|---|---|---|
| No. | of step 1 | Starting material 2 of step 1 |
| 328 | 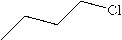 | 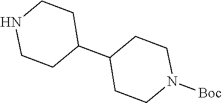 [SMILES: CC(C)(C)OC(=O)N1CCC(C2CCNCC2)CC1] |
| 329 | 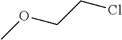 | 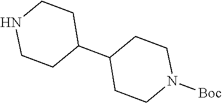 [SMILES: CC(C)(C)OC(=O)N1CCC(C2CCNCC2)CC1] |
| 384 | 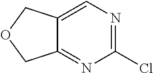 | 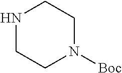 [SMILES: CC(C)(C)OC(=O)N1CCNCC1] |

### Example 265: Synthesis of Compound 265

Example compound 265 was synthesized through substantially the same synthesis method as a synthesis method of example compound 328 except for using (tert-butoxycarbonyl)-L-proline instead of (S)-1-(tert-butoxycarbonyl)azetidin-2-carboxylic acid.

### Example 266: Synthesis of Compound 266

Example compound 266 was synthesized through substantially the same synthesis method as a synthesis method of example compound 329 except for using (tert-butoxycarbonyl)-L-proline instead of (S)-1-(tert-butoxycarbonyl)azetidin-2-carboxylic acid.

### Example 380: Synthesis of Compound 380

Example compound 380 was synthesized through substantially the same synthesis method as a synthesis method of example compound 329 except for using 2-(furan-2-yl)-7-(methylsulfonyl)-[1,2,4]triazolo[1,5-c]pyrimidin-5-amine instead of 2-(furan-2-yl)-5-(methylsulfonyl)-[1,2,4]triazolo[1,5-a][1,3,5]triazin-7-amine.

### Example 383: Synthesis of Compound 383

Example compound 383 was synthesized through substantially the same synthesis method as a synthesis method of example compound 328 except for using 2-(furan-2-yl)-7-(methylsulfonyl)-[1,2,4]triazolo[1,5-c]pyrimidin-5-amine instead of 2-(furan-2-yl)-5-(methylsulfonyl)-[1.2.4]triazolo[1,5-a][1,3,5]triazin-7-amine.

Analysis data of each of the compounds prepared as described above are shown in the table below.

TABLE 27

| Example | | |
|---|---|---|
| No. | Compound Names | Analysis Data |
| 265 | (S)-(1-(7-amino-2-(furan-2-yl)- | ¹H NMR (400 MHz, DMSO-d₆) δ 8.54-8.00 (m, |
| | [1,2,4]triazolo[1,5-a][1,3,5]triazin- | 2H), 7.87 (s, 1H), 7.04 (dt, J = 7.3, 3.6 Hz, 1H), 6.67 |
| | 5-yl)pyrrolidin-2-yl)(1′-butyl-[4,4′- | (d, J = 3.5 Hz, 1H), 5.07-4.93 (m, 1H), 4.54- |
| | bipiperidin]-1-yl)methanone | 4.29 (m, 2H), 4.07 (dt, J = 34.9, 15.8 Hz, 1H), |
| | | 3.63 (ddq, J = 18.6, 13.2, 7.0, 5.9 Hz, 2H), 3.13 (d, |
| | | J = 17.2 Hz, 3H), 2.25 (d, J = 22.5 Hz, 2H), 2.00- |
| | | 1.66 (m, 8H), 1.52 (d, J = 9.0 Hz, 2H), 1.28 |
| | | (ddd, J = 15.6, 12.7, 7.9 Hz, 7H), 1.20-1.04 (m, |
| | | 2H), 0.94-0.85 (m, 3H). LRMS (ES) m/z 522.5 |
| | | (M⁺ + 1). |
| 266 | (S)-(1-(7-amino-2-(furan-2-yl)- | ¹H NMR (400 MHz, DMSO-d₆) δ 5.06-4.90 (m, |
| | [1,2,4]triazolo[1,5-a][1,3,5]triazin- | 1H), 4.35 (t, J = 15.2 Hz, 1H), 4.07 (dt, J = 34.6, |
| | 5-yl)pyrrolidin-2-yl)(1′-(2- | 15.9 Hz, 1H), 3.63 (ddt, J = 18.2, 14.0, 5.1 Hz, 2H), |
| | methoxyethyl)-[4,4′-bipiperidin]-1- | 3.50 (s, 2H), 3.25 (d, J = 2.9 Hz, 4H), 3.16-2.93 |
| | yl) methanone | (m, 3H), 2.27 (s, 1H), 1.91 (dq, J = 11.0, 7.1 Hz, |
| | | 2H), 1.86-1.64 (m, 5H), 1.39-1.05 (m, 7H). |
| | | LRMS (ES) m/z 524.6 (M⁺ + 1). |
| 328 | (S)-(1-(7-amino-2-(furan-2-yl)- | ¹H NMR (400 MHz, DMSO-d₆) δ 8.41 (d, J = |
| | [1,2,4]triazolo[1,5-a][1,3,5]triazin- | 123.2 Hz, 2H), 7.88 (s, 1H), 7.03 (d, J = 3.4 Hz, |
| | 5-yl)azetidin-2-yl)(1′-butyl-[4,4′- | 1H), 6.68 (dd, J = 3.5, 1.8 Hz, 1H), 5.22 (q, J = |
| | bipiperidin]-1-yl)methanone | 8.0, 6.8 Hz, 1H), 4.40 (d, J = 11.9 Hz, 1H), 4.08- |
| | | 3.67 (m, 4H), 2.99 (d, J = 47.1 Hz, 4H), 2.71 (d, J = |
| | | 24.5 Hz, 3H), 2.10 (d, J = 7.9 Hz, 1H), 1.83 (d, |
| | | J = 13.5 Hz, 2H), 1.70 (d, J = 12.7 Hz, 2H), 1.59 |
| | | (dp, J = 10.3, 6.9, 4.5 Hz, 2H), 1.49-1.25 (m, |
| | | 7H), 1.15-0.96 (m, 2H), 0.90 (td, J = 7.4, 1.9 Hz, |
| | | 3H). LRMS (ES) m/z 508.6 (M⁺ + 1). |
| 329 | (S)-(1-(7-amino-2-(furan-2-yl)- | ¹H NMR (400 MHz, DMSO-d₆) δ 8.70-8.01 (m, |
| | [1,2,4]triazolo[1,5-a][1,3,5]triazin- | 2H), 7.86 (s, 1H), 7.06 (dd, J = 11.1, 3.7 Hz, 1H), |
| | 5-yl)azetidin-2-yl)(1′-(2- | 6.71-6.64 (m, 1H), 5.20 (q, J = 6.7, 4.7 Hz, 1H), |
| | methoxyethyl)-[4,4′-bipiperidin]-1- | 4.39 (d, J = 12.6 Hz, 1H), 3.99 (p, J = 7.6 Hz, 5H), |
| | yl)methanone | 3.20 (q, J = 5.3 Hz, 3H), 3.09-2.79 (m, 4H), 2.64 |
| | | (dd, J = 16.9, 8.9 Hz, 2H), 2.14-1.98 (m, 1H), |
| | | 1.83 (t, J = 14.6 Hz, 2H), 1.68 (d, J = 12.2 Hz, 2H), |
| | | 1.57-1.20 (m, 5H), 1.00 (tt, J = 11.2, 5.1 Hz, 2H). |
| | | LRMS (ES) m/z 510.7 (M⁺ + 1). |
| 380 | (S)-(1-(5-amino-2-(furan-2-yl)- | ¹H NMR (400 MHz, DMSO-d₆) δ 7.86 (d, J = 1.6 |
| | [1,2,4]triazolo[1,5-c]pyrimidin-7- | Hz, 1H), 7.62 (s, 2H), 7.06 (d, J = 3.3 Hz, 1H), |
| | yl)azetidin-2-yl)(1′-(2- | 6.66 (dd, J = 3.5, 1.8 Hz, 1H), 5.45 (s, 1H), 5.04 |
| | methoxyethyl)-[4,4′-bipiperidin]-1- | (dt, J = 9.8, 5.1 Hz, 1H), 4.38 (t, J = 13.3 Hz, 1H), |
| | yl)methanone | 3.97-3.62 (m, 3H), 3.45-3.27 (m, 5H), 3.06- |
| | | 2.79 (m, 3H), 2.64 (t, J = 6.7 Hz, 1H), 2.41 (q, J = |
| | | 5.6 Hz, 2H), 2.18 (d, J = 7.0 Hz, 1H), 1.84 (t, J = |
| | | 11.0 Hz, 2H), 1.64 (dd, J = 40.1, 12.4 Hz, 4H), 1.34- |
| | | 0.88 (m, 7H). LRMS (ES) m/z 509.4 (M⁺ + 1). |
| 383 | (S)-(1-(5-amino-2-(furan-2-yl)- | ¹H NMR (400 MHz, DMSO-d₆) δ 7.86 (s, 1H), |
| | [1,2,4]triazolo[1,5-c]pyrimidin-7- | 7.05 (d, J = 3.3 Hz, 1H), 6.70-6.62 (m, 1H), 5.46 |
| | yl)azetidin-2-yl)(1′-butyl-[4,4′- | (d, J = 9.8 Hz, 1H), 5.04 (dt, J = 11.3, 5.4 Hz, 1H), |
| | bipiperidin]-1-yl)methanone | 4.38 (t, J = 13.4 Hz, 1H), 3.96-3.63 (m, 3H), |
| | | 3.07-2.79 (m, 3H), 2.65 (s, 1H), 2.20 (h, J = 6.5, |
| | | 5.7 Hz, 3H), 1.84-1.55 (m, 6H), 1.44-0.94 (m, |
| | | 11H), 0.87 (t, J = 7.3 Hz, 3H). LRMS (ES) m/z |
| | | 507.4 (M⁺ + 1). |
| 384 | 2-(4-((7-amino-2-(furan-2-yl)- | ¹H NMR (400 MHz, DMSO-d₆) δ 8.58-8.03 (m, |
| | [1,2,4]triazolo[1,5-a][1,3,5]triazin- | 3H), 7.86 (d, J = 8.1 Hz, 1H), 7.04 (dd, J = 23.1, |
| | 5-yl)-L-prolyl)piperazin-1-yl)-5,7- | 3.2 Hz, 1H), 6.67 (ddd, J = 11.9, 3.3, 1.7 Hz, 1H), |
| | dihydrofuro[3,4-d]pyrimidine | 5.11-4.99 (m, 1H), 4.88 (m, 4H), 3.98 (m, 1H), |
| | | 3.89-3.55 (m, 8H), 3.43 (m, 1H), 2.37-2.24 (m, |
| | | 1H), 1.92 (d, J = 10.6 Hz, 3H); LRMS (ES) m/z |
| | | 504.25 (M⁺ + 1). |

### Example 229: Synthesis of Compound 229, 1-(4-((7-amino-2-(furan-2-yl)-[1,2,4]triazolo[1,5-a][1,3,5]triazin-5-yl)-L-prolyl)piperazin-1-yl)-3-hydroxy-3-methylbutan-1-one

### [Step 1] Synthesis of tert-butyl 4-(3-hydroxy-3-methylbutanoyl)piperazin-1-carboxylate

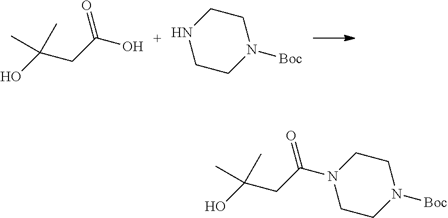

3-Hydroxy-3-methylbutanoic acid (0.315 mL, 2.500 mmol), tert-butyl piperazin-1-carboxylate (0.466 g, 2.500 mmol), 1-ethyl-3-(3-dimethylaminopropyl)carbodiimide hydrochloride (EDC-HCl, 0.959 g, 5.000 mmol), 1H-benzo[d][1,2,3]triazol-1-ol (HOBt, 0.338 g, 2.500 mmol) and N,N-diisopropylethylamine (1.306 mL, 7.500 mmol) were dissolved in dichloromethane (8 mL) at room temperature, after which the resulting solution was stirred at the same temperature for 18 hours. Saturated aqueous solution of ammonium chloride was poured into the reaction mixture, and then an organic layer was extracted with dichloromethane, filtered via a plastic filter to remove a solid residue and an aqueous solution layer therefrom, and concentrated under reduced pressure. An obtained product was used without an additional purification process (title compound, 0.715 g, 99.9%, white solid).

### [Step 2] Synthesis of 3-hydroxy-3-methyl-1-(piperazin-1-yl)butan-1-one hydrochloride

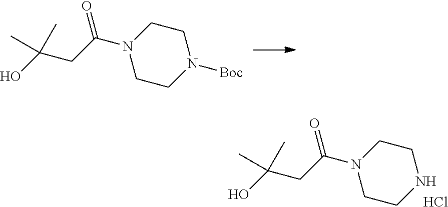 [SMILES: CC(C)(O)CC(=O)N1CCN(C(=O)OC(C)(C)C)CC1 ; CC(C)(O)CC(=O)N1CCNCC1 ; Cl]

Tert-butyl 4-(3-hydroxy-3-methylbutanoyl)piperazin-1-carboxylate (0.715 g, 2.497 mmol) prepared in step 1 and hydrogen chloride (4.00 M solution in 1,4-dioxane, 3.121 mL, 12.484 mmol) were dissolved in dichloromethane (6 mL) at room temperature, after which the resulting solution was stirred at the same temperature for 18 hours. Solvent was removed from the reaction mixture under reduced pressure, after which an obtained product was used without an additional purification process (title compound, 0.555 g, 99.8%, white solid).

### [Step 3] Synthesis of tert-butyl (S)-2-(4-(3-hydroxy-3-methylbutanoyl)piperazin-1-carbonyl)pyrrolidin-1-carboxylate

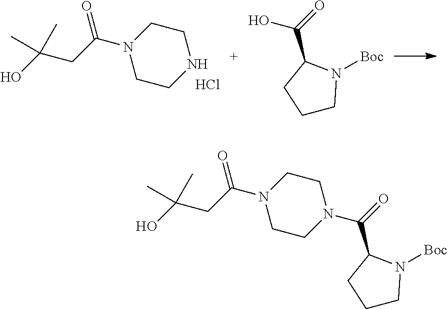 [SMILES: CC(C)(O)CC(=O)N1CCNCC1 ; Cl ; CC(C)(C)OC(=O)N1CCC[C@H]1C(=O)O ; CC(C)(O)CC(=O)N1CCN(C(=O)[C@@H]2CCCN2C(=O)OC(C)(C)C)CC1]

3-Hydroxy-3-methyl-1-(piperazin-1-yl)butan-1-one hydrochloride (0.555 g, 2.492 mmol) prepared in step 2, (tert-butoxycarbonyl)-L-proline (0.536 g, 2.492 mmol), 1-ethyl-3-(3-dimethylaminopropyl)carbodiimide hydrochloride (EDC-HCl, 0.955 g, 4.984 mmol), 1H-benzo[d][1,2,3]triazol-1-ol (HOBt, 0.337 g, 2.492 mmol) and N,N-diisopropylethylamine (1.302 mL, 7.476 mmol) were dissolved in dichloromethane (8 mL) at room temperature, after which the resulting solution was stirred at the same temperature for 18 hours. Saturated aqueous solution of ammonium chloride was poured into the reaction mixture, and then an organic layer was extracted with dichloromethane, filtered via a plastic filter to remove a solid residue and an aqueous solution layer therefrom, and concentrated under reduced pressure. The resulting concentrate was purified via column chromatography (SiO₂, 24 g cartridge; methanol/dichloromethane=0 to 10%) and concentrated to obtain a title compound (0.890 g, 93.1%) as a colorless oil form.

### [Step 4] Synthesis of (S)-3-hydroxy-3-methyl-1-(4-prolylpiperazin-1-yl)butan-1-one hydrochloride

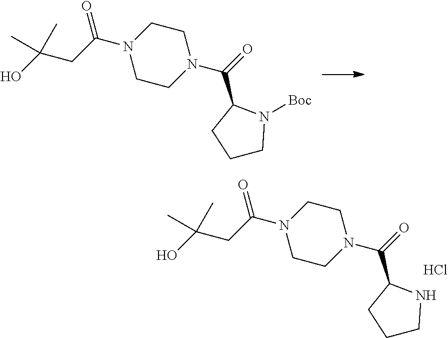

Tert-butyl (S)-2-(4-(3-hydroxy-3-methylbutanoyl)piperazin-1-carbonyl)pyrrolidin-1-carboxylate (0.890 g, 2.321 mmol) prepared in step 3 and hydrogen chloride (4.00 M solution in 1,4-dioxane, 2.321 mL, 9.283 mmol) were dissolved in dichloromethane (5 mL) at room temperature, after which the resulting solution was stirred at 40° C. for two hours to complete the reaction by lowering a temperature to room temperature. Solvent was removed from the reaction mixture under reduced pressure, after which an obtained product was used without an additional purification process (title compound, 0.740 g, 99.7%, white solid).

### [Step 5] Synthesis of 1-(4-((7-amino-2-(furan-2-yl)-[1,2,4]triazolo[1,5-a][1,3,5]triazin-5-yl)-L-prolyl)piperazin-1-yl)-3-hydroxy-3-methylbutan-1-one

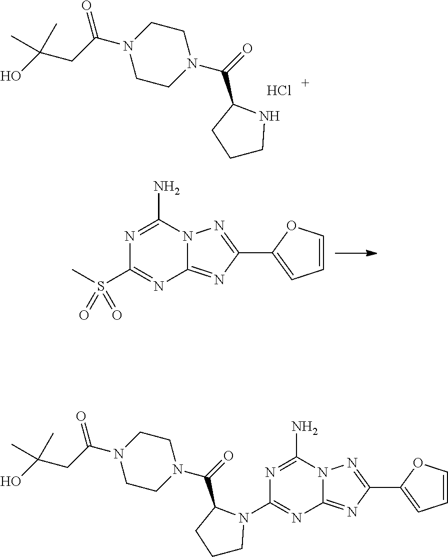 [SMILES: CC(C)(O)CC(=O)N1CCN(C(=O)[C@@H]2CCCN2)CC1 ; Cl ; CS(=O)(=O)c1nc(N)n2nc(-c3ccco3)nc2n1 ; CC(C)(O)CC(=O)N1CCN(C(=O)[C@@H]2CCCN2c2nc(N)n3nc(-c4ccco4)nc3n2)CC1]

(S)-3-Hydroxy-3-methyl-1-(4-prolylpiperazin-1-yl)butan-1-one hydrochloride (0.150 g, 0.469 mmol) prepared in step 4, 2-(furan-2-yl)-5-(methylsulfonyl)-[1,2,4]triazolo[1,5-a][1,3,5]triazin-7-amine (0.131 g, 0.469 mmol) and sodium hydrogen carbonate (0.079 g, 0.938 mmol) were dissolved in acetonitrile (2 mL) at room temperature, after which the resulting solution was stirred at 50° C. for 18 hours to complete the reaction by lowering a temperature to room temperature. Solvent was removed from the reaction mixture under reduced pressure, after which the resulting concentrate was purified via column chromatography (SiO₂, 12 g cartridge; methanol/dichloromethane=0 to 5%) and concentrated to obtain a product, after which the obtained product was purified again via chromatography (SiO₂ plate, 20×20×1 mm; methanol/dichloromethane=10%) and concentrated to obtain a title compound (0.117 g, 51.8%) as a white solid form.

¹H NMR (400 MHz, DMSO-d₆) δ 8.63-8.00 (m, 2H), 7.91-7.84 (m, 1H), 7.10-7.01 (m, 1H), 6.72-6.64 (m, 1H), 5.09-4.94 (m, 1H), 4.88-4.72 (m, 1H), 3.89-3.38 (m, 10H), 2.61-2.53 (m, 2H), 2.35-2.17 (m, 1H), 1.98-1.77 (m, 3H), 1.27-1.13 (m, 6H); LRMS (ES) m/z 484.3 (M⁺+1).

### Examples 230 and 237

Example compounds 230 and 237 were each synthesized through substantially the same synthesis method as a synthesis method of example compound 229 except for using the starting materials of the following table instead of 3-hydroxy-3-methylbutanoic acid of step 1.

TABLE 28

| Example | Starting |
|---|---|
| No. | Material |
| 230 | 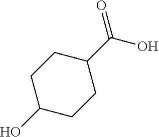 |
| 237 | 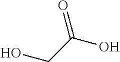 |

### Example 262: Synthesis of Compound 262

Example compound 262 was synthesized through substantially the same synthesis method as a synthesis method of example compound 268 except for using 2-(furan-2-yl)-5-(methylsulfonyl)-3a,7a-dihydrothiazolo[5,4-d]pyrimidin-7-amine instead of 2-(furan-2-yl)-5-(methylsulfonyl)-[1,2,4]triazolo[1,5-a][1,3,5]triazin-7-amine of step 5.

### Example 263: Synthesis of Compound 263

Example compound 263 was synthesized through substantially the same synthesis method as a synthesis method of example compound 229 except for using 2-(furan-2-yl)-5-(methylsulfonyl)-3a,7a-dihydrooxazolo[5,4-d]pyrimidin-7-amine instead of 2-(furan-2-yl)-5-(methylsulfonyl)-[1,2,4]triazolo[1,5-a][1,3,5]triazin-7-amine of step 5.

### Example 267: Synthesis of Compound 267

Example compound 267 was synthesized through substantially the same synthesis method as a synthesis method of example compound 229 except for using 2-hydroxy-2-methylpropanoic acid instead of 3-hydroxy-3-methylbutanoic acid of step 1 and using (S)-1-(tert-butoxycarbonyl)azetidin-2-carboxylic acid instead of (tert-butoxycarbonyl)-L-proline of step 3.

### Example 268: Synthesis of Compound 268

Example compound 268 was synthesized through substantially the same synthesis method as a synthesis method of example compound 229 except for using (S)-1-(tert-butoxycarbonyl)azetidin-2-carboxylic acid instead of (tert-butoxycarbonyl)-L-proline of step 3.

### Example 274: Synthesis of Compound 274

Example compound 274 was synthesized through substantially the same synthesis method as a synthesis method of example compound 229 except for using 2-hydroxy-2-methylpropanoic acid instead of 3-hydroxy-3-methylbutanoic acid of step 1.

### Example 275: Synthesis of Compound 275

Example compound 275 was synthesized through substantially the same synthesis method as a synthesis method of example compound 268 except for using 2-(furan-2-yl)-5-(methylsulfonyl)-3a,7a-dihydrothiazolo[5,4-d]pyrimidin-7-amine instead of 2-(furan-2-yl)-5-(methylsulfonyl)-[1,2,4]triazolo[1,5-a][1,3,5]triazin-7-amine of step 5.

### Example 290: Synthesis of Compound 290

Example compound 290 was synthesized through substantially the same synthesis method as a synthesis method of example compound 229 except for using tert-butyl 4,4′-bipiperidin-1-carboxylate instead of tert-butyl piperazin-1-carboxylate of step 1.

### Example 327: Synthesis of Compound 327

Example compound 327 was synthesized through substantially the same synthesis method as a synthesis method of example compound 290 except for using (S)-1-(tert-butoxycarbonyl)azetidin-2-carboxylic acid instead of (tert-butoxycarbonyl)-L-proline.

### Example 292: Synthesis of Compound 292

Example compound 292 was synthesized through substantially the same synthesis method as a synthesis method of example compound 229 except for using 2-(furan-2-yl)-7-(methylsulfonyl)-[1,2,4]triazolo[1,5-c]pyrimidin-5-amine instead of 2-(furan-2-yl)-5-(methylsulfonyl)-[1,2,4]triazolo[1,5-a][1,3,5]triazin-7-amine of step 5.

### Example 315: Synthesis of Compound 315

Example compound 315 was synthesized through substantially the same synthesis method as a synthesis method of example compound 268 except for using 2-(methylfuran-2-yl)-5-(methylsulfonyl)-[1,2,4]triazolo[1,5-a][1,3,5]triazin-7-amine instead of 2-(furan-2-yl)-5-(methylsulfonyl)-[1,2,4]triazolo[1,5-a][1,3,5]triazin-7-amine of step 5.

### Example 340: Synthesis of Compound 340

Example compound 340 was synthesized through substantially the same synthesis method as a synthesis method of example compound 262 except for using (tert-butoxycarbonyl)-L-alanine instead of (tert-butoxycarbonyl)-L-proline.

### Examples 368, 369 and 370

Example compounds 368, 369 and 370 were each synthesized through substantially the same synthesis method as a synthesis method of example compound 229 except for using the compounds of the following table as N-protected amino acid instead of (tert-butoxycarbonyl)-L-proline of step 3.

TABLE 29

| Example No. | Amino acid |
|---|---|
| 368 | 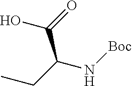 |
| 369 | 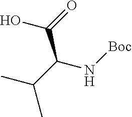 |
| 370 | 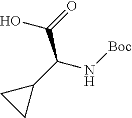 [SMILES: CC(C)(C)OC(=O)N[C@H](C(=O)O)C1CC1] |

Analysis data of each of the compounds prepared as described above are shown in the table below.

TABLE 30

| Example | | |
|---|---|---|
| No. | Compound Names | Analysis Data |
| 230 | 4-((7-amino-2-(furan-2-yl)- | ¹H NMR (400 MHz, DMSO-d₆) δ 8.68-7.99 |
| | [1,2,4]triazolo[1,5-a][1,3,5]triazin-5- | (m, 2H), 7.92-7.80 (m, 1H), 7.12-6.98 (m, |
| | yl)-L-prolyl)piperazin-1-yl)(4- | 1H), 6.71-6.61 (m, 1H), 5.08-4.91 (m, 1H), |
| | hydroxycyclohexyl)methanone | 4.61-4.24 (m, 1H), 3.89-3.37 (m, 11H), |
| | | 2.71-2.56 (m, 1H), 2.35-2.17 (m, 1H), 2.03- |
| | | 1.74 (m, 5H), 1.73-1.58 (m, 2H), 1.57- |
| | | 1.14 (m, 4H); LRMS (ES) m/z 492.3 (M⁺ + 1). |
| 237 | 1-(4-((7-amino-2-(furan-2-yl)- | ¹H NMR (400 MHz, DMSO-d₆) δ 8.71-8.06 |
| | [1,2,4]triazolo[1,5-a][1,3,5]triazin-5- | (m, 2H), 7.93-7.83 (m, 1H), 7.15-6.99 (m, |
| | yl)-L-prolyl)piperazin-1-yl)-2- | 1H), 6.73-6.61 (m, 1H), 5.08-4.91 (m, 1H), |
| | hydroxyethan-1-one | 4.76-4.58 (m, 1H), 4.27-4.09 (m, 2H), |
| | | 3.85-3.36 (m, 10H), 2.35-2.18 (m, 1H), |
| | | 1.98-1.78 (m, 3H); LRMS (ES) m/z 442.3 |
| | | (M⁺ + 1). |
| 262 | 1-(4-((7-amino-2-(furan-2- | ¹H NMR (400 MHz, DMSO-d₆) δ 7.90 (s, 1H), |
| | yl)thiazolo[5,4-d]pyrimidin-5-yl)-L- | 7.38-6.91 (m, 3H), 6.72 (s, 1H), 5.07-4.90 |
| | prolyl)piperazin-1-yl)-3-hydroxy-3- | (m, 1H), 4.81 (d, J = 6.9 Hz, 1H), 3.93-3.40 |
| | methylbutan-1-one | (m, 9H), 3.27-3.14 (m, 1H), 2.59-2.52 (m, |
| | | 2H), 2.30-2.15 (m, 1H), 2.10-1.77 (m, 3H), |
| | | 1.21 (s, 6H); LRMS (ES) m/z 500.3 (M⁺ + 1). |
| 263 | 1-(4-((7-amino-2-(furan-2- | ¹H NMR (400 MHz, DMSO-d₆) δ 7.95 (d, J = |
| | yl)oxazolo[5,4-d]pyrimidin-5-yl)-L- | 4.4 Hz, 1H), 7.46-6.91 (m, 3H), 6.74 (s, 1H), |
| | prolyl)piperazin-1-yl)-3-hydroxy-3- | 5.02-4.88 (m, 1H), 4.88-4.71 (m, 1H), 3.87- |
| | methylbutan-1-one | 3.37 (m, 10H), 2.55-2.52 (m, 2H), 2.31- |
| | | 2.15 (m, 1H), 2.01-1.75 (m, 3H), 1.21 (d, J = |
| | | 4.8 Hz, 6H); LRMS (ES) m/z 484.4 (M⁺ + 1). |
| 267 | (S)-1-(4-(1-(7-amino-2-(furan-2-yl)- | ¹H NMR (400 MHz, DMSO-d₆) δ 8.49 (d, J = |
| | [1,2,4]triazolo[1,5-a][1,3,5]triazin-5- | 173.8 Hz, 4H), 7.88 (s, 1H), 7.13-6.95 (m, |
| | yl)azetidine-2-carbonyl)piperazin-1- | 1H), 6.72-6.56 (m, 1H), 5.52 (d, J = 13.9 Hz, |
| | yl)-2-hydroxy-2-methylpropan-1-one | 1H), 5.25 (dd, J = 9.2, 5.3 Hz, 1H), 4.09- |
| | | 3.94 (m, 2H), 3.62 (p, J = 6.7 Hz, 6H), 3.47 |
| | | (d, J = 24.3 Hz, 4H), 1.34 (s, 3H), 1.24 (s, 3H). |
| | | LRMS (ES) m/z 456.3 (M⁺ + 1). |
| 268 | (S)-1-(4-(1-(7-amino-2-(furan-2-yl)- | ¹H NMR (400 MHz, DMSO-d₆) δ 8.44 (d, J = |
| | [1,2,4]triazolo[1,5-a][1,3,5]triazin-5- | 117.7 Hz, 2H), 7.92-7.79 (m, 1H), 7.11-6.98 |
| | yl)azetidine-2-carbonyl)piperazin-1- | (m, 1H), 6.68 (td, J = 4.4, 3.5, 1.8 Hz, 1H), |
| | yl)-3-hydroxy-3-methylbutan-1-one | 5.26 (q, J = 8.1, 7.6 Hz, 1H), 4.79 (d, J = 3.3 |
| | | Hz, 1H), 4.02 (s, 2H), 3-76-3.40 (m, 8H), |
| | | 3.18 (d, J = 5.2 Hz, 2H), 2.65 (d, J = 15.7 Hz, |
| | | 1H), 2.18 (s, 1H), 1.20 (s, 6H). LRMS (ES) |
| | | m/z 470.4 (M⁺ + 1). |
| 274 | 1-(4-((7-amino-2-(furan-2-yl)- | ¹H NMR (400 MHz, DMSO-d₆) δ 8.59-8.03 |
| | [1,2,4]triazolo[1,5-a][1,3,5]triazin-5- | (m, 2H), 7.87 (d, J = 4.4 Hz, 1H), 7.13-7.01 |
| | yl)-L-prolyl)piperazin-1-yl)-2- | (m, 1H), 6.68 (dd, J = 5.4, 3.2 Hz, 1H), 5.50 |
| | hydroxy-2-methylpropan-1-one | (d, J = 26.0 Hz, 1H), 5.01 (dd, J = 13.2, 8.9 |
| | | Hz, 1H), 4.11 (q, J = 5.6 Hz, 1H), 3.80-3.48 |
| | | (m, 7H), 3.18 (dd, J = 5.2, 1.7 Hz, 2H), 2.29 |
| | | (td, J = 15.1, 13.7, 8.5 Hz, 1H), 1.93 (dt, J = |
| | | 20.8, 10.7 Hz, 3H), 1.36 (s, 6H). LRMS (ES) |
| | | m/z 470.6 (M⁺ + 1). |
| 275 | (S)-1-(4-(1-(7-amino-2-(furan-2- | ¹H NMR (400 MHz, DMSO-d₆) δ 7.90 (d, J = |
| | yl)thiazolo[5,4-d]pyrimidin-5- | 1.7 Hz, 1H), 7.30 (s, 2H), 7.07 (d, J = 3.5 Hz, |
| | yl)azetidin-2-carbonyl)piperazin-1- | 1H), 6.72 (dd, J = 3.5, 1.8 Hz, 1H), 5.19 (q, J = |
| | yl)-3-hydroxy-3-methylbutan-1-one | 7.0 Hz, 1H), 4.80 (d, J = 2.8 Hz, 1H), 3.97 |
| | | (dt, J = 17.5, 6.6 Hz, 2H), 3.61 (d, J = 46.9 Hz, |
| | | 9H), 2.57 (s, 1H), 2.19 (s, 1H), 1.20 (s, 6H). |
| | | LRMS (ES) m/z 486.4 (M⁺ + 1). |
| 290 | 1-(1′-((7-amino-2-(furan-2-yl)- | ¹H NMR (400 MHz, DMSO-d₆) δ 8.28 (d, J = |
| | [1,2,4]triazolo[1,5-a][1,3,5]triazin-5- | 123.6 Hz, 2H), 7.87 (s, 1H), 7.04 (dt, J = 14.3, |
| | yl)-L-prolyl)-[4,4′-bipiperidin]-1-yl)- | 4.0 Hz, 1H), 6.68 (dt, J = 3.5, 1.9 Hz, 1H), |
| | 3-hydroxy-3-methylbutan-1-one | 5.08-4.90 (m, 2H), 4.61-4.28 (m, 2H), |
| | | 4.06 (dd, J = 31.7, 14.9 Hz, 2H), 3.72-3.53 |
| | | (m, 2H), 3.05 (ddd, J = 61.6, 26.5, 13.9 Hz, |
| | | 2H), 2.36-2.17 (m, 1H), 1.99-1.59 (m, 8H), |
| | | 1.17 (s, 13H). LRMS (ES) m/z 566.7 (M⁺ + 1). |
| 292 | 1-(4-((5-amino-2-(furan-2-yl)- | ¹H NMR (400 MHz, DMSO-d₆) δ 7.86 (d, J = |
| | [1,2,4]triazolo[1,5-c]pyrimidin-7-yl)- | 0.9 Hz, 1H), 7.47 (brs, 2H), 7.06 (d, J = 3.2 |
| | L-prolyl)piperazin-1-yl)-3-hydroxy-3- | Hz, 1H), 6.67 (dd, J = 3.4, 1.8 Hz, 1H), 5.64 |
| | methylbutan-1-one | (brs, 1H), 4.99 (brs, 1H), 4.81 (d, J = 9.0 Hz, |
| | | 1H), 3.85-3.67 (m, 2H), 3.65-3.38 (m, |
| | | 8H), 2.32-2.13 (m, 1H), 2.08-1.73 (m, 4H), |
| | | 1.21 (d, J = 3.7 Hz, 7H); LRMS (ES) m/z |
| | | 483.6 (M⁺ + 1). |
| 315 | (S)-1-(4-(1-(7-amino-2-(5- | ¹H NMR (400 MHz, DMSO-d₆) δ 8.41 (d, J = |
| | methylfuran-2-yl)-[1,2,4]triazolo[1,5- | 104.7 Hz, 2H), 6.99-6.87 (m, 1H), 6.29 (d, J = |
| | a][1,3,5]triazin-5-yl)azetidine-2- | 3.3 Hz, 1H), 5.25 (q, J = 8.5 Hz, 1H), 4.79 |
| | carbonyl)piperazin-1-yl)-3-hydroxy- | (d, J = 3.5 Hz, 1H), 4.11 (d, J = 79.7 Hz, 2H), |
| | 3-methylbutan-1-one | 3.77-3.38 (m, 8H), 2.63 (s, 1H), 2.36 (s, |
| | | 3H), 2.19 (d, J = 11.3 Hz, 1H), 1.20 (s, 8H), |
| | | 0.90-0.77 (m, 1H). LRMS (ES) m/z 484.4 |
| | | (M⁺ + 1). |
| 327 | (S)-1-(1′-(1-(7-amino-2-(furan-2-yl)- | ¹H NMR (400 MHz, DMSO-d₆) δ 8.41 (d, J = |
| | [1,2,4]triazolo[1,5-a][1,3,5]triazin-5- | 121.9 Hz, 2H), 7.88 (d, J = 1.8 Hz, 1H), 7.78- |
| | yl)azetidine-2-carbonyl)-[4,4′- | 7.65 (m, 1H), 7.04 (d, J = 10.7 Hz, 1H), 6.68 |
| | bipiperidin]-1-yl)-3-hydroxy-3- | (dd, J = 3.6, 1.9 Hz, 1H), 5.21 (d, J = 10.6 Hz, |
| | methylbutan-1-one | 1H), 4.94 (d, J = 4.8 Hz, 1H), 4.55-4.31 (m, |
| | | 2H), 4.26-4.17 (m, 1H), 4.08-3.91 (m, 3H), |
| | | 3.89-3.67 (m, 1H), 2.96 (dt, J = 25.5, 15.3 |
| | | Hz, 2H), 2.09 (s, 1H), 1.69 (d, J = 13.0 Hz, |
| | | 5H), 1.28 (d, J = 29.4 Hz, 6H), 1.16 (s, 6H), |
| | | 1.13-0.98 (m, 3H), 0.90-0.79 (m, 3H). |
| | | LRMS (ES) m/z 552.8 (M⁺ + 1). |
| 340 | 1-(4-((7-amino-2-(furan-2- | ¹H NMR (400 MHz, DMSO-d₆) δ 7.89 (d, J = |
| | yl)thiazolo[5,4-d]pyrimidin-5-yl)-L- | 1.3 Hz, 1H), 7.22 (brs, 2H), 7.07 (d, J = 3.4 |
| | alanyl)piperazin-1-yl)-3-hydroxy-3- | Hz, 1H), 6.87 (brs, 1H), 6.72 (dd, J = 3.5, 1.8 |
| | methylbutan-1-one | Hz, 1H), 4.93-4.70 (m, 2H), 3.87-3.43 (m, |
| | | 7H), 3.32-3.18 (m, 1H), 2.50-2.42 (m, 2H), |
| | | 1.26 (d, J = 6.8 Hz, 3H), 1.19 (s, 6H); LRMS |
| | | (ES) m/z 474.5 (M⁺ + 1). |
| 368 | (S)-1-(4-(2-((7-amino-2-(furan-2-yl)- | ¹H NMR (400 MHz, DMSO-d₆) δ 8.54-8.02 |
| | [1,2,4]triazolo[1,5-a][1,3,5]triazin-5- | (m, 2H), 7.89-7.83 (m, 1H), 7.64-7.42 (m, |
| | yl)amino)butanoyl)piperazin-1-yl)-3- | 1H), 7.10-7.02 (m, 1H), 6.68 (dd, J = 3.2, 1.7 |
| | hydroxy-3-methylbutan-1-one | Hz, 1H), 4.87-4.67 (m, 2H), 3.81-3.36 (m, |
| | | 8H), 2.49-2.41 (m, 2H), 1.80-1.58 (m, |
| | | 2H), 1.19 (s, 6H), 0.92 (t, J = 7.3 Hz, 3H); |
| | | LRMS (ES) m/z 472.3 (M⁺ + 1). |
| 369 | 1-(4-((7-amino-2-(furan-2-yl)- | ¹H NMR (400 MHz, DMSO-d₆) δ 8.45-8.03 |
| | [1,2,4]triazolo[1,5-a][1,3,5]triazin-5- | (m, 2H), 7.91-7.83 (m, 1H), 7.63-7.39 (m, |
| | yl)-L-valyl)piperazin-1-yl)-3-hydroxy- | 1H), 7.10-7.02 (m, 1H), 6.71-6.64 (m, 1H), |
| | 3-methylbutan-1-one | 4.78 (d, J = 9.7 Hz, 1H), 4.65 (dt, J = 31.4, 8.4 |
| | | Hz, 1H), 3.91-3.37 (m, 8H), 2.48-2.40 (m, |
| | | 2H), 2.17-2.01 (m, 1H), 1.18 (s, 6H), 0.93 |
| | | (dd, J = 15.2, 6.7 Hz, 6H); LRMS (ES) m/z |
| | | 486.4 (M⁺ + 1). |
| 370 | (S)-1-(4-(2-((7-amino-2-(furan-2-yl)- | ¹H NMR (400 MHz, DMSO-d₆) δ 8.49-8.06 |
| | [1,2,4]triazolo[1,5-a][1,3,5]triazin-5- | (m, 2H), 7.90-7.84 (m, 1H), 7.72-7.54 (m, |
| | yl)amino)-2- | 1H), 7.10-7.01 (m, 1H), 6.67 (dd, J = 3.2, 1.7 |
| | cyclopropylacetyl)piperazin-1-yl)-3- | Hz, 1H), 4.78 (s, 1H), 4.48-4.33 (m, 1H), |
| | hydroxy-3-methylbutan-1-one | 3.81-3.38 (m, 8H), 2.50-2.41 (m, 2H), 1.24 |
| | | (s, 1H), 1.19 (s, 6H), 0.54-0.31 (m, J = 20.8, |
| | | 17.0 Hz, 4H); LRMS (ES) m/z 484.4 (M⁺ + 1). |

### Example 276: Synthesis of Compound 276, (S)-(1-(7-amino-2-(furan-2-yl)-[1,2,4]triazolo[1,5-a][1,3,5]triazin-5-yl)pyrrolidin-2-yl)(4-(2,4-difluorophenyl)-1,4-diazepan-1-yl)methanone

### [Step 1] Synthesis of tert-butyl 4-(2,4-difluorophenyl)-1,4-diazepan-1-carboxylate

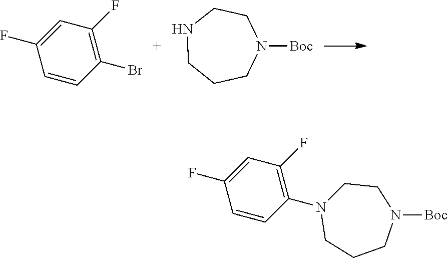 [SMILES: Fc1ccc(Br)c(F)c1 ; CC(C)(C)OC(=O)N1CCCNCC1 ; CC(C)(C)OC(=O)N1CCCN(c2ccc(F)cc2F)CC1]

1-Bromo-2,4-difluorobenzene (0.386 g, 2.000 mmol), tert-butyl 1,4-diazepan-1-carboxylate (0.401 g, 2.000 mmol), chloro(2-dicyclohexylphosphino-2′,6′-diisopropyl-1,1′-diphenyl)[2-(2′-amino-1,1′-diphenyl)]palladium (II, 0.155 g, 0.200 mmol) and cesium carbonate (1.955 g, 6.000 mmol) were dissolved in tetrahydrofuran (10 mL) at room temperature, after which the resulting solution was stirred at 80° C. for 18 hours to complete the reaction by lowering a temperature to room temperature. Water was poured into the reaction mixture and an organic layer was extracted with dichloromethane. The organic layer was washed with saturated aqueous solution of sodium chloride, dehydrated with anhydrous sodium sulfate, filtered, and concentrated under reduced pressure. The resulting concentrate was purified via column chromatography (SiO₂, 12 g cartridge; dichloromethane/methanol=0 to 30%) and concentrated to obtain a title compound (0.624 g, 99.9%) as a light yellow liquid form.

### [Step 2] Synthesis of 1-(2,4-difluorophenyl)-1,4-diazepane hydrochloride

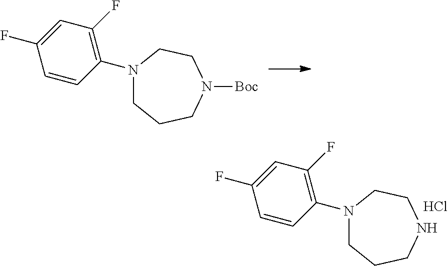 [SMILES: CC(C)(C)OC(=O)N1CCCN(c2ccc(F)cc2F)CC1 ; Fc1ccc(N2CCCNCC2)c(F)c1 ; Cl]

Tert-butyl 4-(2,4-difluorophenyl)-1,4-diazepan-1-carboxylate (0.624 g, 1.998 mmol) prepared in step 1 and hydrochloric acid (4.00 M solution in 1,4-dioxane, 1.998 mL, 7.991 mmol) were dissolved in dichloromethane (5 mL) at room temperature, after which the resulting solution was stirred at the same temperature for five hours. Solvent was removed from the reaction mixture under reduced pressure, after which an obtained product was used without an additional purification process (title compound, 0.248 g, 49.9%, white solid).

### [Step 3] Synthesis of tert-butyl (S)-2-(4-(2,4-difluorophenyl)-1,4-diazepan-1-carbonyl)pyrrolidin-1-carboxylate

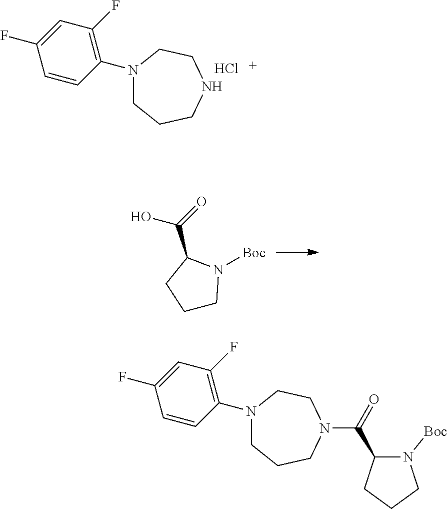 [SMILES: Fc1ccc(N2CCCNCC2)c(F)c1 ; Cl ; CC(C)(C)OC(=O)N1CCC[C@H]1C(=O)O ; CC(C)(C)OC(=O)N1CCC[C@H]1C(=O)N1CCCN(c2ccc(F)cc2F)CC1]

1-(2,4-Difluorophenyl)-1,4-diazepane hydrochloride (0.497 g, 1.998 mmol) prepared in step 2, (tert-butoxycarbonyl)-L-proline (0.430 g, 1.998 mmol), 2,4,6-tripropyl-1,3,5,2,4,6-trioxatriphosphinane 2,4,6-trioxide (T3P, 50.00% solution in EtOAc, 1.833 mL, 2.998 mmol) and N,N-diisopropylethylamine (1.392 mL, 7.994 mmol) were dissolved in dichloromethane (5 mL) at room temperature, after which the resulting solution was stirred at the same temperature for 18 hours. Aqueous solution of N-sodium hydrogen carbonate was poured into the reaction mixture, and then an organic layer was extracted with dichloromethane, filtered via a plastic filter to remove a solid residue and an aqueous solution layer therefrom, and concentrated under reduced pressure. An obtained product was used without an additional purification process (title compound, 0.409 g, 50.0%, light yellow liquid).

### [Step 4] Synthesis of (S)-1-(2,4-difluorophenyl)-4-prolyl-1,4-diazepane hydrochloride

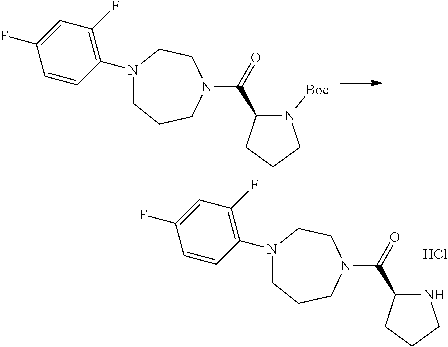

Tert-butyl (S)-2-(4-(2,4-difluorophenyl)-1,4-diazepan-1-carbonyl)pyrrolidin-1-carboxylate (0.409 g, 0.999 mmol) prepared in step 3 and hydrochloric acid (4.00 M solution in 1,4-dioxane, 0.999 mL, 3.995 mmol) were dissolved in dichloromethane (5 mL) at room temperature, after which the resulting solution was stirred at the same temperature for 18 hours. Solvent was removed from the reaction mixture under reduced pressure, after which an obtained product was used without an additional purification process (title compound, 0.345 g, 99.9%, white solid).

### [Step 5] Synthesis of (S)-(1-(7-amino-2-(furan-2-yl)-[1,2,4]triazolo[1,5-a][1,3,5]triazin-5-yl)pyrrolidin-2-yl)(4-(2,4-difluorophenyl)-1,4-diazepan-1-yl)methanone

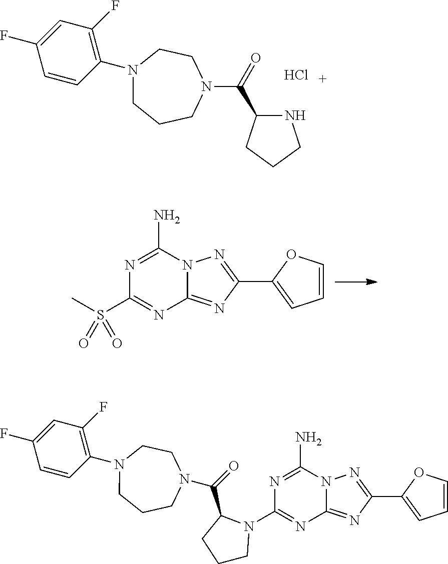 [SMILES: O=C([C@@H]1CCCN1)N1CCCN(c2ccc(F)cc2F)CC1 ; Cl ; CS(=O)(=O)c1nc(N)n2nc(-c3ccco3)nc2n1 ; Nc1nc(N2CCC[C@H]2C(=O)N2CCCN(c3ccc(F)cc3F)CC2)nc2nc(-c3ccco3)nn12]

(S)-1-(2,4-Difluorophenyl)-4-prolyl-1,4-diazepane hydrochloride (0.345 g, 0.998 mmol) prepared in step 4, 2-(furan-2-yl)-5-(methylsulfonyl)-[1,2,4]triazolo[1,5-a][1,3,5]triazin-7-amine (0.280 g, 0.998 mmol) and (S)-(1-(7-amino-2-(furan-2-yl)-[1,2,4]triazolo[1,5-a][1,3,5]triazin-5-yl)pyrrolidin-2-yl) (4-(2,4-difluorophenyl)-1,4-diazepan-1-yl)methanone (1.525 g, 2.993 mmol) were dissolved in acetonitrile (5 mL) at room temperature, after which the resulting solution was stirred at 70° C. for 18 hours to complete the reaction by lowering a temperature to room temperature. The reaction mixture was filtered via a celite pad to remove a solid therefrom, after which solvent was removed from the resulting filtrate without the solid under reduced pressure. Then, the resulting concentrate was purified via column chromatography (SiO₂, 12 g cartridge; dichloromethane/methanol=0 to 30%) and concentrated to obtain a title compound (0.011 g, 2.1%) as a white solid form.

¹H NMR (400 MHz, DMSO-d₆) δ 8.54-8.06 (m, 2H), 7.87 (s, 1H), 7.22-6.83 (m, 4H), 6.68 (ddd, J=5.2, 3.5, 1.6 Hz, 1H), 5.03-4.91 (m, 1H), 3.87-3.37 (m, 10H), 2.34-2.09 (m, 2H), 1.97-1.78 (m, 4H); LRMS (ES) m/z 510.5 (M⁺+1).

### Example 42: Synthesis of Compound 42, (S)-(1-(7-amino-2-(furan-2-yl)-[1,2,4]triazolo[1,5-a][1,3,5]triazin-5-yl)pyrrolidin-2-yl)(4-(2-ethyl-2-hydroxybutyl)piperazin-1-yl)methanone

### [Step 1] Synthesis of tert-butyl 4-(2-ethyl-2-hydroxybutyl)piperazin-1-carboxylate

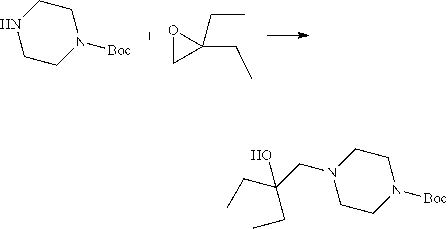

Tert-butyl piperazin-1-carboxylate (2.000 g, 10.738 mmol), 2,2-diethyloxirane (2.151 g, 21.475 mmol) and potassium carbonate (5.936 g, 42.951 mmol) were mixed in ethanol (10 mL)/water (2 mL) at room temperature, irradiated with microwave, and heated at 110° C. for one hour to complete the reaction by lowering a temperature to room temperature. Solvent was removed from the reaction mixture under reduced pressure, after which water was poured into the resulting concentrate and the organic layer was extracted with ethyl acetate. The organic layer was washed with saturated aqueous solution of sodium chloride, dehydrated with anhydrous sodium sulfate, filtered, and concentrated under reduced pressure. An obtained product was used without an additional purification process (title compound, 2.800 g, 91.0%, white solid).

### [Step 2] Synthesis of 3-(piperazin-1-ylmethyl)pentan-3-ol

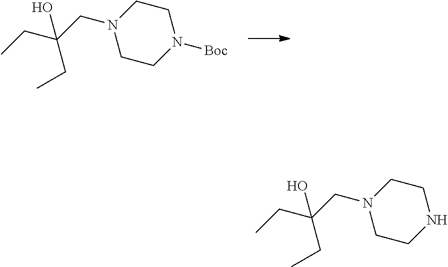 [SMILES: CCC(O)(CC)CN1CCN(C(=O)OC(C)(C)C)CC1 ; CCC(O)(CC)CN1CCNCC1]

Tert-butyl 4-(2-ethyl-2-hydroxybutyl)piperazin-1-carboxylate (0.600 g, 2.095 mmol) prepared in step 1 and hydrochloric acid (4.00 M solution in dioxane, 5.237 mL, 20.948 mmol) were dissolved in dichloromethane (10 mL) at room temperature, after which the resulting solution was stirred at the same temperature for 18 hours. Solvent was removed from the reaction mixture under reduced pressure, after which an obtained product was used without an additional purification process (title compound, 0.300 g, 76.9%, white solid).

### [Step 3] Synthesis of tert-butyl (S)-2-(4-(2-ethyl-2-hydroxybutyl)piperazin-1-carbonyl)pyrrolidin-1-carboxylate

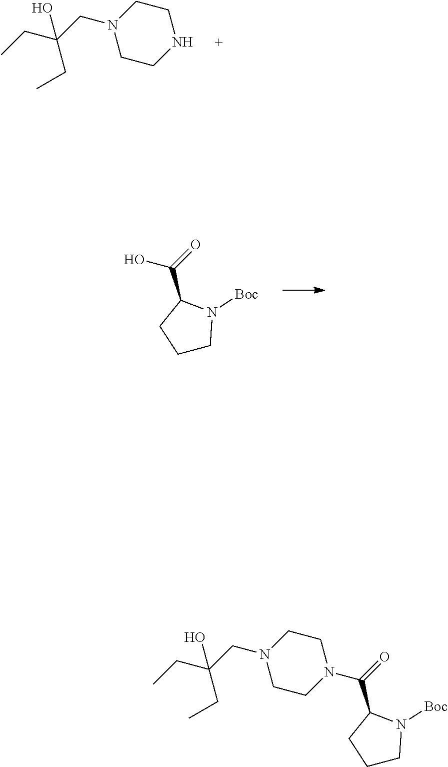 [SMILES: CCC(O)(CC)CN1CCNCC1 ; CC(C)(C)OC(=O)N1CCC[C@H]1C(=O)O ; CCC(O)(CC)CN1CCN(C(=O)[C@@H]2CCCN2C(=O)OC(C)(C)C)CC1]

3-(Piperazin-1-ylmethyl)pentan-3-ol (0.500 g, 2.684 mmol) prepared in step 2, (tert-butoxycarbonyl)-L-proline (1.155 g, 5.368 mmol), [dimethylamino(triazolo[4,5-b]pyridin-3-yloxy)methylidene]-dimethylazanium; hexafluorophosphate (2.041 g, 5.368 mmol) and N,N-diisopropylethylamine (0.935 mL, 5.368 mmol) were dissolved in N,N-dimethylformamide (20 mL) at room temperature, after which the resulting solution was stirred at the same temperature for 18 hours. Water was poured into the reaction mixture and an organic layer was extracted with ethyl acetate. The organic layer was washed with saturated aqueous solution of sodium chloride, dehydrated with anhydrous magnesium sulfate, filtered, and concentrated under reduced pressure. The resulting concentrate was purified via column chromatography (SiO₂, 12 g cartridge; methanol/dichloromethane=0 to 10%) and concentrated to obtain a title compound (0.200 g, 19.4%) as a white solid form.

### [Step 4] Synthesis of (S)-1-(2-ethyl-2-hydroxybutyl)-4-prolylpiperazine

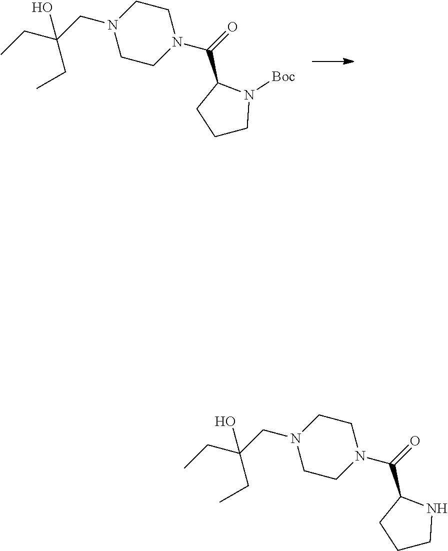 [SMILES: CCC(O)(CC)CN1CCN(C(=O)[C@@H]2CCCN2C(=O)OC(C)(C)C)CC1 ; CCC(O)(CC)CN1CCN(C(=O)[C@@H]2CCCN2)CC1]

Tert-butyl (S)-2-(4-(2-ethyl-2-hydroxybutyl)piperazin-1-carbonyl)pyrrolidin-1-carboxylate (0.200 g, 0.501 mmol) prepared in step 3 and hydrochloric acid (4.00 M solution in dioxane, 1.252 mL, 5.006 mmol) were dissolved in dichloromethane (10 mL) at room temperature, after which the resulting solution was stirred at the same temperature for 18 hours. Solvent was removed from the reaction mixture under reduced pressure, after which an obtained product was used without an additional purification process (title compound, 0.140 g, 93.4%, white solid).

### [Step 5] Synthesis of (S)-(1-(7-amino-2-(furan-2-yl)-[1,2,4]triazolo[1,5-a][1,3,5]triazin-5-yl)pyrrolidin-2-yl)(4-(2-ethyl-2-hydroxybutyl)piperazin-1-yl)methanone

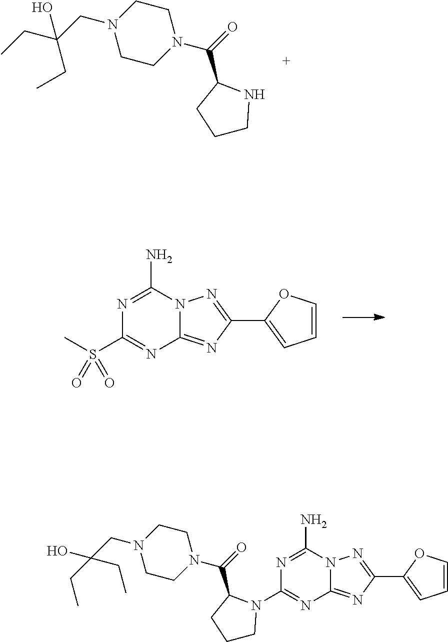 [SMILES: CCC(O)(CC)CN1CCN(C(=O)[C@@H]2CCCN2)CC1 ; CS(=O)(=O)c1nc(N)n2nc(-c3ccco3)nc2n1 ; CCC(O)(CC)CN1CCN(C(=O)[C@@H]2CCCN2c2nc(N)n3nc(-c4ccco4)nc3n2)CC1]

(S)-1-(2-Ethyl-2-hydroxybutyl)-4-prolylpiperazine (0.200 g, 0.706 mmol) prepared in step 4, 2-(furan-2-yl)-5-(methylsulfonyl)-[1,2,4]triazolo[1,5-a][1,3,5]triazin-7-amine (0.099 g, 0.353 mmol) and triethylamine (0.197 mL, 1.411 mmol) were dissolved in dimethylsulfoxide (5 mL) at room temperature, after which the resulting solution was stirred at the same temperature for 18 hours. Water was poured into the reaction mixture and an organic layer was extracted with ethyl acetate. The organic layer was washed with saturated aqueous solution of sodium chloride, dehydrated with anhydrous magnesium sulfate, filtered, and concentrated under reduced pressure. The resulting concentrate was purified via chromatography (SiO₂ plate, 20×20×1 mm; methanol/dichloromethane=10%) and concentrated to obtain a title compound (0.006 g, 1.8%) as a white solid form.

¹H NMR (400 MHz, DMSO-d₆) δ 8.54-8.06 (m, 2H), 7.87 (s, 1H), 7.06 (d, J=3.1 Hz, 1H), 6.68 (d, J=3.3 Hz, 1H), 5.03-4.91 (m, 1H), 3.92-3.79 (m, 1H), 3.72-3.39 (m, 6H), 2.89-2.55 (m, 2H), 2.43-2.14 (m, 5H), 2.01-1.75 (m, 3H), 1.57-1.26 (m, 4H), 0.80 (t, J=7.0 Hz, 6H); LRMS (ES) m/z 484.4 (M⁺+1).

### Example 88: Synthesis of Compound 88

Example compound 88 was synthesized through substantially the same synthesis method as a synthesis method of example compound 42 except for using 2-(5-methylfuran-2-yl)-5-(methylsulfonyl)-[1,2,4]triazolo[1,5-a][1,3,5]triazin-7-amine instead of 2-(furan-2-yl)-5-(methylsulfonyl)-[1,2,4]triazolo[1,5-a][1,3,5]triazin-7-amine of step 5.

### Example 185: Synthesis of Compound 185

Example compound 185 was synthesized through substantially the same synthesis method as a synthesis method of example compound 42 except for using (S)-2-((tert-butoxycarbonyl)amino)-2-cyclohexylacetic acid instead of (tert-butoxycarbonyl)-L-proline of step 3.

### Example 347: Synthesis of Compound 347

Example compound 347 was synthesized through substantially the same synthesis method as a synthesis method of example compound 42 except for using (S)-1-(tert-butoxycarbonyl)azetidin-2-carboxylic acid instead of (tert-butoxycarbonyl)-L-proline of step 3.

### Example 348: Synthesis of Compound 348

Example compound 348 was synthesized through substantially the same synthesis method as a synthesis method of example compound 42 except for using (tert-butoxycarbonyl)-L-valine instead of (tert-butoxycarbonyl)-L-proline of step 3.

### Example 244: Synthesis of Compound 244

Example compound 244 was synthesized through substantially the same synthesis method as a synthesis method of example compound 42 except for using tert-butyl [4,4′-bipiperidin]-1-carboxylate instead of tert-butyl piperazin-1-carboxylate of step 1 and using 2,2-dimethyloxirane instead of 2,2-diethyloxirane.

### Example 333: Synthesis of Compound 333

Example compound 333 was synthesized through substantially the same synthesis method as a synthesis method of example compound 244 except for using (tert-butoxycarbonyl)-L-alanine instead of (tert-butoxycarbonyl)-L-proline.

### Example 373: Synthesis of Compound 373

Example compound 373 was synthesized through substantially the same synthesis method as a synthesis method of example compound 244 except for using tert-butyl (R)-2-methylpiperazin-1-carboxylate instead of tert-butyl piperazin-1-carboxylate.

### Example 381: Synthesis of Compound 381

Example compound 333 was synthesized through substantially the same synthesis method as a synthesis method of example compound 244 except for using (S)-1-(tert-butoxycarbonyl)azetidin-2-carboxylic acid instead of (tert-butoxycarbonyl)-L-proline.

Analysis data of each of the compounds prepared as described above are shown in the table below.

TABLE 31

| Example | | |
|---|---|---|
| No. | Compound Names | Analysis Data |
| 88 | (S)-(1-(7-amino-2-(5- | ¹H NMR (400 MHz, DMSO-d₆) δ 8.52-8.07 (m, |
| | methylfuran-2-yl)- | 1H), 6.94 (d, J = 3.3 Hz, 1H), 6.29 (ddt, J = 2.9, |
| | [1,2,4]triazolo[1,5-a][1,3,5]triazin- | 2.0, 1.0 Hz, 1H), 5.03-4.93 (m, 1H), 3.90-3.81 |
| | 5-yl)pyrrolidin-2-yl)(4-(2-ethyl-2- | (m, 1H), 3.71-3.48 (m, 5H), 3.31-3.17 (m, 1H), |
| | hydroxybutyl)piperazin-1- | 2.85-2.13 (m, 10H), 1.95-1.76 (m, 3H), 1.50- |
| | yl) methanone | 1.29 (m, 4H), 0.84-0.68 (m, 6H); LRMS (ES) |
| | | m/z 498.6 (M⁺ + 1). |
| 185 | (S)-2-((7-amino-2-(furan-2-yl)- | ¹H NMR (400 MHz, Chloroform-d) δ 9.60 (s, |
| | [1,2,4]triazolo[1,5-a][1,3,5]triazin- | 1H), 8.46 (d, J = 9.4 Hz, 1H), 7.60 (d, J = 1.7 Hz, |
| | 5-yl)amino)-2-cyclohexyl-1-(4-(2- | 1H), 7.20 (d, J = 3.5 Hz, 1H), 6.58 (dd, J = 3.5, 1.7 |
| | ethyl-2-hydroxybutyl)piperazin-1- | Hz, 1H), 6.23 (s, 1H), 5.17 (t, J = 9.4 Hz, 1H), 4.12- |
| | yl)ethan-1-one | 3.58 (m, 5H), 2.77 (dd, J = 32.7, 10.3 Hz, 3H), |
| | | 2.62 (t, J = 4.9 Hz, 3H), 2.38 (s, 2H), 1.82 (dd, J = |
| | | 20.7, 10.7 Hz, 3H), 1.74-1.57 (m, 5H), 1.55- |
| | | 1.38 (m, 5H), 1.25-0.96 (m, 7H), 0.88 (t, J = 7.4 |
| | | Hz, 8H). LRMS (ES) m/z 526.6(M⁺ + 1). |
| 347 | (S)-(1-(7-amino-2-(furan-2-yl)- | ¹H NMR (400 MHz, DMSO-d₆) δ 8.41 (m, 2H), |
| | [1,2,4]triazolo[1,5-a][1,3,5]triazin- | 7.88 (d, J = 0.9 Hz, 1H), 7.05 (s, 1H), 6.68 (dd, J = |
| | 5-yl)azetidin-2-yl)(4-(2-ethyl-2- | 3.4, 1.8 Hz, 1H), 5.21 (m, 1H), 4.01 (s, 2H), 3.85 |
| | hydroxybutyl)piperazin-1- | (m, 1H), 3.69-3.39 (m, 3H), 2.65 (m, 3H), 2.41- |
| | yl)methanone | 2.28 (m, 1H), 2.46-2.29 (m, 2H), 2.24 (s, 2H), |
| | | 2.09 (s, 1H), 1.57-1.32 (m, 4H), 0.79 (t, J = 7.4 |
| | | Hz, 6H); LRMS (ES) m/z 470.5 (M⁺ + 1). |
| 348 | (S)-2-((7-amino-2-(furan-2-yl)- | ¹H NMR (400 MHz, DMSO-d₆) δ 8.21 (s, 2H), |
| | [1,2,4]triazolo[1,5-a][1,3,5]triazin- | 7.87 (s, 1H), 7.37 (dd, J = 75.1, 8.4 Hz, 1H), 7.07 |
| | 5-yl)amino)-1-(4-(2-ethyl-2- | (d, J = 3.3 Hz, 1H), 6.68 (dd, J = 3.2, 1.7 Hz, 1H), |
| | hydroxybutyl)piperazin-1-yl)-3- | 4.69 (m, 1H), 3.87 (m, 1H), 3.77-3.39 (m, 5H), |
| | methylbutan-1-one | 2.62 (m, 1H), 2.34 (m, 2H), 2.21 (s, 2H), 2.08 (m, |
| | | 1H), 1.53-1.27 (m, 4H), 0.97-0.70 (m, 12H); |
| | | LRMS (ES) m/z 486.6 (M⁺ + 1). |
| 244 | (S)-(1-(7-amino-2-(furan-2-yl)- | ¹H NMR (400 MHz, DMSO-d₆) δ 8.22 (d, J = |
| | [1,2,4]triazolo[1,5-a][1,3,5]triazin- | 131.4 Hz, 2H), 7.88-7.72 (m, 1H), 7.13-6.99 |
| | 5-yl)pyrrolidin-2-yl)(1′-(2- | (m, 1H), 6.67 (dt, J = 9.1, 2.5 Hz, 1H), 4.97 (ddt, |
| | hydroxy-2-methylpropyl)-[4,4′- | J = 16.7, 7.2, 3.9 Hz, 1H), 4.33 (t, J = 13.6 Hz, 1H), |
| | bipiperidin]-1-yl)methanone | 4.03 (dt, J = 35.6, 16.5 Hz, 2H), 3.08-2.88 (m, |
| | | 2H), 2.32-2.03 (m, 4H), 1.98-1.50 (m, 6H), |
| | | 1.41-1.14 (m, 3H), 1.08 (d, J = 7.9 Hz, 6H), 1.01- |
| | | 0.85 (m, 1H). LRMS (ES) m/z 538.4 (M⁺ + 1). |
| 333 | (S)-2-((7-amino-2-(furan-2-yl)- | ¹H NMR (400 MHz, DMSO-d₆) δ 8.26 (s, 2H), |
| | [1,2,4]triazolo[1,5-a][1,3,5]triazin- | 7.87 (d, J = 1.7 Hz, 1H), 7.36 (dd, J = 20.4, 7.5 Hz, |
| | 5-yl)amino)-1-(1′-(2-hydroxy-2- | 1H), 7.10-6.95 (m, 1H), 6.68 (dd, J = 3.4, 1.8 Hz, |
| | methylpropyl)-[4,4′-bipiperidin]- | 1H), 4.94-4.82 (m, 1H), 4.40 (d, J = 12.8 Hz, |
| | 1-yl)propan-1-one | 1H), 4.01 (d, J = 13.8 Hz, 1H), 3.01 (d, J = 24.3 |
| | | Hz, 3H), 2.14 (d, J = 49.8 Hz, 4H), 1.84-1.51 (m, |
| | | 4H), 1.37-1.15 (m, 7H), 1.07 (s, 8H). LRMS (ES) |
| | | m/z 512.7 (M⁺ + 1). |
| 373 | ((S)-1-(7-amino-2-(furan-2-yl)- | ¹H NMR (400 MHz, DMSO-d₆) δ 8.58-8.00 (m, |
| | [1,2,4]triazolo[1,5-a][1,3,5]triazin- | 2H), 7.90-7.81 (m, 1H), 7.06 (d, J = 3.4 Hz, 1H), |
| | 5-yl)pyrrolidin-2-yl)((R)-4-(2- | 6.68 (ddd, J = 5.9, 3.6, 1.9 Hz, 1H), 5.07-4.84 |
| | hydroxy-2-methylpropyl)-2- | (m, 1H), 4.44-3.99 (m, 3H), 3.86-3.57 (m, 3H), |
| | methylpiperazin-1-yl)methanone | 3.03-2.76 (m, 3H), 2.36-2.13 (m, 4H), 2.04- |
| | | 1.70 (m, 4H), 1.54 (dd, J = 18.1, 6.6 Hz, 2H), 1.21- |
| | | 1.06 (m, 7H). LRMS (ES) m/z 470.3 (M⁺ + 1). |
| 381 | (S)-(1-(5-amino-2-(furan-2-yl)- | ¹H NMR (400 MHz, DMSO-d₆) δ 7.86 (d, J = 1.7 |
| | [1,2,4]triazolo[1,5-c]pyrimidin-7- | Hz, 1H), 7.61 (s, 2H), 7.05 (d, J = 3.3 Hz, 1H), 6.66 |
| | yl)azetidin-2-yl)(1′-(2-hydroxy-2- | (dd, J = 3.4, 1.8 Hz, 1H), 5.46 (d, J = 10.1 Hz, 1H), |
| | methylpropyl)-[4,4′-bipiperidin]- | 5.05 (dd, J = 9.4, 5.1 Hz, 1H), 4.40 (d, J = 13.3 Hz, |
| | 1-yl)methanone | 1H), 4.06-3.63 (m, 4H), 2.95 (dq, J = 23.9, 14.2, |
| | | 13.6 Hz, 3H), 2.65 (dt, J = 9.4, 4.8 Hz, 1H), 2.24- |
| | | 2.10 (m, 3H), 2.02 (q, J = 10.8 Hz, 2H), 1.75- |
| | | 1.64 (m, 2H), 1.56 (d, J = 12.6 Hz, 2H), 1.30-1.13 |
| | | (m, 4H), 1.09-0.91 (m, 9H). LRMS (ES) m/z |
| | | 523.3 (M⁺ + 1). |

### Example 223: Synthesis of Compound 223, (S)-(1-(7-amino-2-(furan-2-yl)-[1,2,4]triazolo[1,5-a][1,3,5]triazin-5-yl)pyrrolidin-2-yl)(4-(2,2-difluoroethyl)piperazin-1-yl)methanone

### [Step 1] Synthesis of tert-butyl 4-(2,2-difluoroethyl)piperazin-1-carboxylate

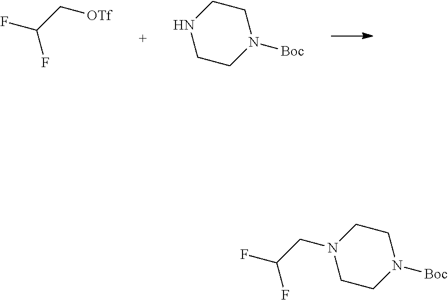 [SMILES: O=S(=O)(OCC(F)F)C(F)(F)F ; CC(C)(C)OC(=O)N1CCNCC1 ; CC(C)(C)OC(=O)N1CCN(CC(F)F)CC1]

2,2-Difluoroethyl trifluoromethanesulfonate (95.00% solution 0.279 mL, 1.899 mmol), tert-butyl piperazin-1-carboxylate (0.354 g, 1.899 mmol) and N,N-diisopropylethylamine (0.331 mL, 1.899 mmol) were dissolved in tetrahydrofuran (5 mL) at room temperature, after which the resulting solution was stirred at the same temperature for 18 hours. Solvent was removed from the reaction mixture under reduced pressure, after which aqueous solution of N-ammonium chloride was poured into the resulting concentrate, and then an organic layer was extracted with dichloromethane, filtered via a plastic filter to remove a solid residue and an aqueous solution layer therefrom, and concentrated under reduced pressure. The resulting concentrate was purified via column chromatography (SiO₂, 12 g cartridge; dichloromethane/methanol=0 to 20%), and concentrated to obtain a title compound (0.411 g, 86.1%) as a white solid form.

### [Step 2] Synthesis of 1-(2,2-difluoroethyl)piperazine hydrochloride

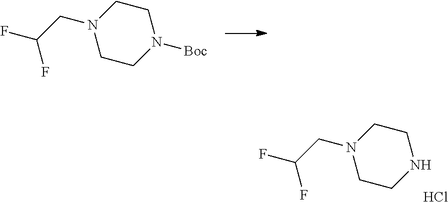 [SMILES: CC(C)(C)OC(=O)N1CCN(CC(F)F)CC1 ; FC(F)CN1CCNCC1 ; Cl]

Tert-butyl 4-(2,2-difluoroethyl)piperazin-1-carboxylate (0.250 g, 0.999 mmol) prepared in step 1 and hydrochloric acid (4.00 M solution in 1,4-dioxane, 0.999 mL, 3.995 mmol) were dissolved in dichloromethane (5 mL) at room temperature, after which the resulting solution was stirred at the same temperature for 18 hours. Solvent was removed from the reaction mixture under reduced pressure, after which an obtained product was used without an additional purification process (title compound, 0.186 g, 99.8%, white solid).

### [Step 3] Synthesis of tert-butyl (S)-2-(4-(2,2-difluoroethyl)piperazin-1-carbonyl)pyrrolidin-1-carboxylate

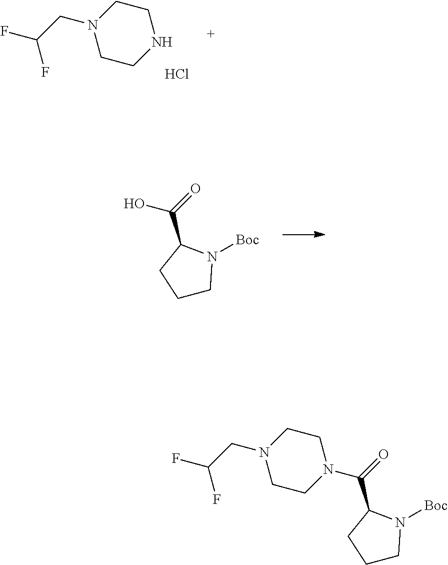 [SMILES: FC(F)CN1CCNCC1 ; Cl ; CC(C)(C)OC(=O)N1CCC[C@H]1C(=O)O ; CC(C)(C)OC(=O)N1CCC[C@H]1C(=O)N1CCN(CC(F)F)CC1]

1-(2,2-Difluoroethyl)piperazine hydrochloride (3.614 g, 19.365 mmol) prepared in step 2, (tert-butoxycarbonyl)-L-proline (4.168 g, 19.365 mmol), 2,4,6-tripropyl-1,3,5,2,4,6-trioxatriphosphinane 2,4,6-trioxide (T3P, 50.00% solution in EtOAc, 17.767 mL, 29.048 mmol) and N,N-diisopropylethylamine (10.119 mL, 58.095 mmol) were dissolved in dichloromethane (130 mL) at room temperature, after which the resulting solution was stirred at the same temperature for 18 hours. Aqueous solution of N-sodium hydrogen carbonate was poured into the reaction mixture and an organic layer was extracted with dichloromethane. The organic layer was washed with saturated aqueous solution of sodium chloride, dehydrated with anhydrous sodium sulfate, filtered, and concentrated under reduced pressure. The resulting concentrate was purified via column chromatography (SiO₂, 12 g cartridge; dichloromethane/methanol) and concentrated to obtain a title compound (5.360 g, 79.7%) as a yellow liquid form.

### [Step 4] Synthesis of (S)-1-(2,2-difluoroethyl)-4-prolylpiperazine hydrochloride

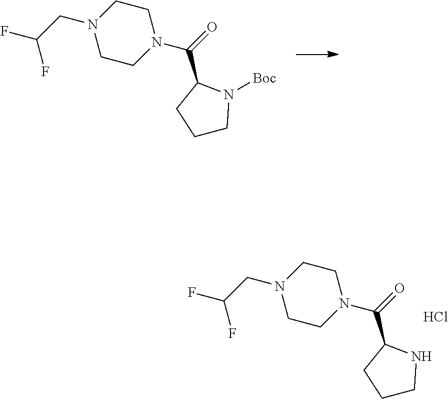 [SMILES: CC(C)(C)OC(=O)N1CCC[C@H]1C(=O)N1CCN(CC(F)F)CC1 ; O=C([C@@H]1CCCN1)N1CCN(CC(F)F)CC1 ; Cl]

Tert-butyl (S)-2-(4-(2,2-difluoroethyl)piperazin-1-carbonyl)pyrrolidin-1-carboxylate (5.360 g, 15.428 mmol) prepared in step 3 and hydrogen chloride (4.00 M solution in 1,4-dioxane, 15.428 mL, 61.714 mmol) were dissolved in dichloromethane (65 mL) at room temperature, after which the resulting solution was stirred at 40° C. for five hours to complete the reaction by lowering a temperature to room temperature. Solvent was removed from the reaction mixture under reduced pressure, after which an obtained product was used without an additional purification process (title compound, 4.377 g, 100.0%, white solid).

### [Step 5] Synthesis of (S)-(1-(7-amino-2-(furan-2-yl)-[1,2,4]triazolo[1,5-a][1,3,5]triazin-5-yl)pyrrolidin-2-yl)(4-(2,2-difluoroethyl)piperazin-1-yl)methanone

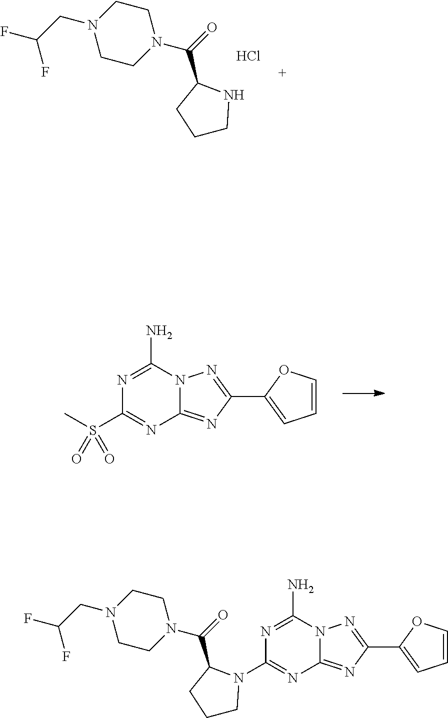 [SMILES: O=C([C@@H]1CCCN1)N1CCN(CC(F)F)CC1 ; Cl ; CS(=O)(=O)c1nc(N)n2nc(-c3ccco3)nc2n1 ; Nc1nc(N2CCC[C@H]2C(=O)N2CCN(CC(F)F)CC2)nc2nc(-c3ccco3)nn12]

(S)-1-(2,2-Difluoroethyl)-4-prolylpiperazine hydrochloride 4377 g, 15.426 mmol) prepared in step 4, 2-(furan-2-yl)-5-(methylsulfonyl)-[1,2,4]triazolo[1,5-a][1,3,5]triazin-7-amine (4.323 g, 15.426 mmol) and sodium hydrogen carbonate (3.887 g, 46.277 mmol) were dissolved in acetonitrile (90 mL) at room temperature, after which the resulting solution was stirred at 80° C. for 18 hours to complete the reaction by lowering a temperature to room temperature. The reaction mixture was filtered via a celite pad to remove a solid therefrom, after which solvent was removed from the resulting filtrate without the solid under reduced pressure. Then, the resulting concentrate was purified via column chromatography (SiO₂, 12 g cartridge; dichloromethane/methanol=0 to 20%) and concentrated to obtain a title compound (4.240 g, 61.4%) as a light yellow solid form.

¹H NMR (400 MHz, DMSO-d₆) δ 8.33 (d, J=126.9 Hz, 2H), 7.90-7.84 (m, 1H), 7.06 (ddd, J=7.7, 3.4, 0.9 Hz, 1H), 6.68 (dt, J=3-4, 1.6 Hz, 1H), 6.40-6.02 (m, 1H), 4.99 (ddd, J=12.5, 8.6, 3.1 Hz, 1H), 4.12 (q, J=5.3 Hz, 1H), 3.70-3.49 (m, 5H), 3.17 (d, J=5.1 Hz, 2H), 2.82 (tdd, J=17.4, 10.3, 4.2 Hz, 3H), 2.58 (d, J=9.2 Hz, 1H), 2.48-2.37 (m, 1H), 2.26 (ddd, J=12.2, 8.1, 3.8 Hz, 1H), 1.96-1.79 (m, 3H). LRMS (ES) m/z 448.3 (M⁺+1).

### Example 224: Synthesis of Compound 224, (S)-(1-(7-amino-2-(furan-2-yl)-[1,2,4]triazolo[1,5-a][1,3,5]triazin-5-yl)pyrrolidin-2-yl)(4-(2,2-difluoropropyl)piperazin-1-yl)methanone

### [Step 1] Synthesis of tert-butyl 4-(2,2-difluoropropyl)piperazin-1-carboxylate

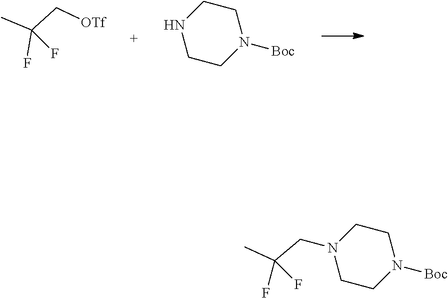 [SMILES: CC(F)(F)COS(=O)(=O)C(F)(F)F ; CC(C)(C)OC(=O)N1CCNCC1 ; CC(F)(F)CN1CCN(C(=O)OC(C)(C)C)CC1]

2,2-Difluoropropyl trifluoromethanesulfonate 0.45 g, 1.999 mmol), tert-butyl piperazin-1-carboxylate (0.372 g, 1.999 mmol) and N,N-diisopropylethylamine (0.348 mL, 1.999 mmol) were dissolved in tetrahydrofuran (5 mL) at room temperature, after which the resulting solution was stirred at the same temperature for 18 hours. Solvent was removed from the reaction mixture under reduced pressure, after which aqueous solution of N-ammonium chloride was poured into the resulting concentrate, and then an organic layer was extracted with dichloromethane, filtered via a plastic filter to remove a solid residue and an aqueous solution layer therefrom, and concentrated under reduced pressure. The resulting concentrate was purified via column chromatography (SiO₂, 12 g cartridge; dichloromethane/methanol=0 to 20%) and concentrated to obtain a title compound (0.423 g, 80.1%) as a white solid form.

### [Step 2] Synthesis of 1-(2,2-difluoropropyl)piperazine hydrochloride

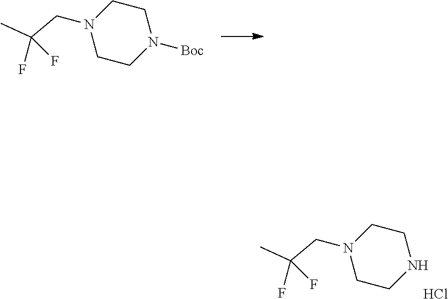 [SMILES: CC(F)(F)CN1CCN(C(=O)OC(C)(C)C)CC1 ; CC(F)(F)CN1CCNCC1 ; Cl]

Tert-butyl 4-(2,2-difluoropropyl)piperazin-1-carboxylate (0.264 g, 0.999 mmol) prepared in step 1 and hydrochloric acid (4.00 M solution in 1,4-dioxane, 0.999 mL, 3.995 mmol) were dissolved in dichloromethane (5 mL) at room temperature, after which the resulting solution was stirred at the same temperature for 18 hours. Solvent was removed from the reaction mixture under reduced pressure, after which an obtained product was used without an additional purification process (title compound, 0.200 g, 99.8%, white solid).

### [Step 3] Synthesis of tert-butyl (S)-2-(4-(2,2-difluoropropyl)piperazin-1-carbonyl)pyrrolidin-1-carboxylate

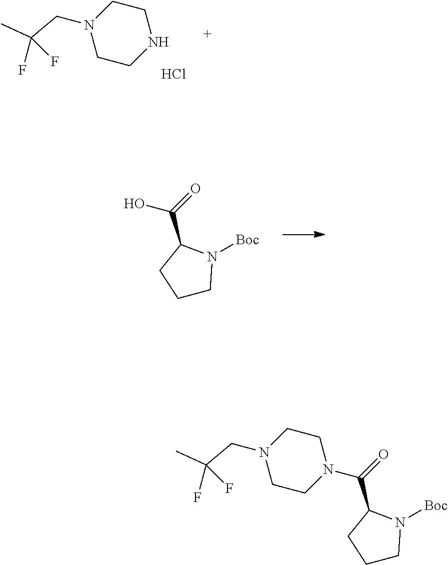 [SMILES: CC(F)(F)CN1CCNCC1 ; Cl ; CC(C)(C)OC(=O)N1CCC[C@H]1C(=O)O ; CC(F)(F)CN1CCN(C(=O)[C@@H]2CCCN2C(=O)OC(C)(C)C)CC1]

1-(2,2-Difluoropropyl)piperazine hydrochloride (6.000 g, 29.901 mmol) prepared in step 2, (tert-butoxycarbonyl)-L-proline (6.436 g, 29.901 mmol), 2,4,6-tripropyl-1,3,5,2,4,6-trioxatriphosphinane 2,4,6-trioxide (T3P, 50.00% solution in EtOAc, 27.434 mL, 44.852 mmol) and N,N-diisopropylethylamine (15.624 mL, 89.704 mmol) were dissolved in dichloromethane (150 mL) at room temperature, after which the resulting solution was stirred at the same temperature for 18 hours. Solvent was removed from the reaction mixture under reduced pressure, after which saturated aqueous solution of N-sodium hydrogen carbonate was poured into the resulting concentrate and an organic layer was extracted with dichloromethane. The organic layer was washed with saturated aqueous solution of sodium chloride, dehydrated with anhydrous sodium sulfate, filtered, and concentrated under reduced pressure. The resulting concentrate was purified via column chromatography (SiO₂, 12 g cartridge; dichloromethane/methanol=0 to 30%) and concentrated to obtain a title compound (8.030 g, 74.3%) as a light yellow liquid form.

### [Step 4] Synthesis of (S)-1-(2,2-difluoropropyl)-4-prolylpiperazine hydrochloride

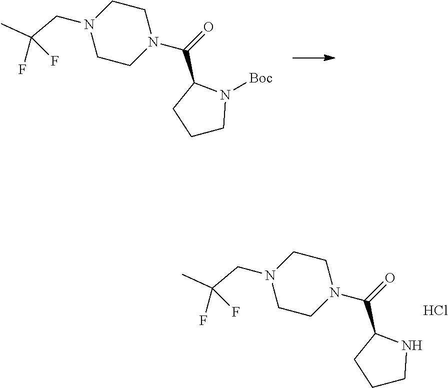 [SMILES: CC(F)(F)CN1CCN(C(=O)[C@@H]2CCCN2C(=O)OC(C)(C)C)CC1 ; CC(F)(F)CN1CCN(C(=O)[C@@H]2CCCN2)CC1 ; Cl]

Tert-butyl (S)-2-(4-(2,2-difluoropropyl)piperazin-1-carbonyl)pyrrolidin-1-carboxylate (8.030 g, 22.217 mmol) prepared in step 3 and hydrochloric acid (4.00 M solution in 1,4-dioxane, 22.217 mL, 88.869 mmol) were dissolved in dichloromethane (100 mL) at room temperature, after which the resulting solution was stirred at the same temperature for 18 hours. Solvent was removed from the reaction mixture under reduced pressure, after which an obtained product was used without an additional purification process (title compound, 6.615 g, 100.0%, light yellow solid).

### [Step 5] Synthesis of (S)-(1-(7-amino-2-(furan-2-yl)-[1,2,4]triazolo[1,5-a][1,3,5]triazin-5-yl)pyrrolidin-2-yl)(4-(2,2-difluoropropyl)piperazin-1-yl)methanone

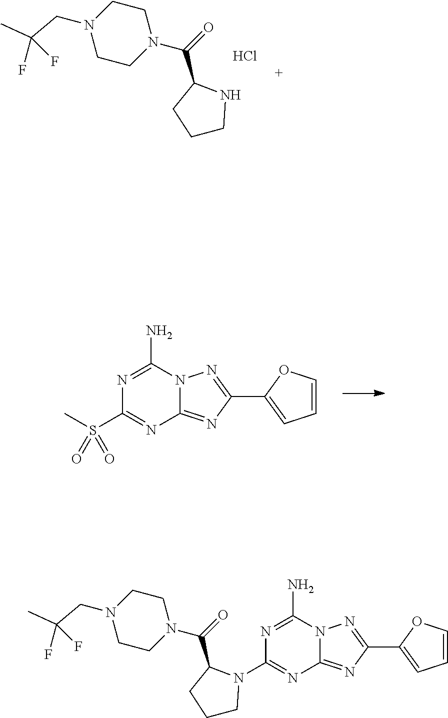 [SMILES: CC(F)(F)CN1CCN(C(=O)[C@@H]2CCCN2)CC1 ; Cl ; CS(=O)(=O)c1nc(N)n2nc(-c3ccco3)nc2n1 ; CC(F)(F)CN1CCN(C(=O)[C@@H]2CCCN2c2nc(N)n3nc(-c4ccco4)nc3n2)CC1]

(S)-1-(2,2-Difluoropropyl)-4-prolylpiperazine hydrochloride (5.000 g, 16.791 mmol) prepared in step 4, 2-(furan-2-yl)-5-(methylsulfonyl)-[1,2,4]triazolo[1,5-a][1,3,5]triazin-7-amine (4.706 g, 16.791 mmol) and sodium hydrogen carbonate (4.232 g, 50.374 mmol) were dissolved in acetonitrile (95 mL) at room temperature, after which the resulting solution was stirred at 70° C. for 18 hours to complete the reaction by lowering a temperature to room temperature. The reaction mixture was filtered via a celite pad to remove a solid therefrom, after which solvent was removed from the resulting filtrate without the solid under reduced pressure. Then, the resulting concentrate was purified via column chromatography (SiO₂, 12 g cartridge; dichloromethane/methanol=0 to 30%) and concentrated to obtain a title compound (2.920 g, 37.7%) as a white solid form.

¹H NMR (400 MHz, DMSO-d₆) δ 8.33 (d, J=124.7 Hz, 2H), 7.88 (s, 1H), 7.06 (s, 1H), 6.68 (s, 1H), 4.98 (t, J=10.6 Hz, 1H), 3.63 (t, J=9.8 Hz, 5H), 3.17 (d, J=5.0 Hz, 1H), 2.80 (td, J=13.9, 7.5 Hz, 3H), 2.60 (s, 1H), 2.46-2.19 (m, 2H), 2.00-1.77 (m, 3H), 1.66 (t, J=19.1 Hz, 3H). LRMS (ES) m/z 462.4 (M⁺+1).

### Example 294: Synthesis of Compound 294, (S)-(1-(5-amino-2-(furan-2-yl)-[1,2,4]triazolo[1,5-c]pyrimidin-7-yl)pyrrolidin-2-yl)(4-(2,2-difluoropropyl)piperazin-1-yl)methanone

### [Step 1] Synthesis of tert-butyl 4-(2,2-difluoropropyl)piperazin-1-carboxylate

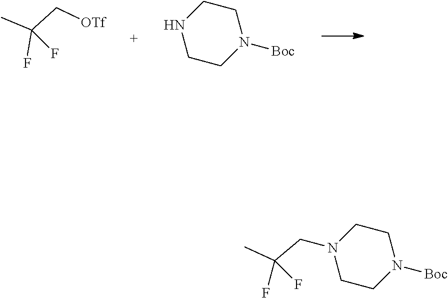 [SMILES: CC(F)(F)COS(=O)(=O)C(F)(F)F ; CC(C)(C)OC(=O)N1CCNCC1 ; CC(F)(F)CN1CCN(C(=O)OC(C)(C)C)CC1]

Tert-butyl piperazin-1-carboxylate (0.559 g, 3.000 mmol), 2,2-difluoropropyl trifluoromethanesulfonate (0.447 mL, 3.000 mmol) and potassium carbonate (0.829 g, 6.000 mmol) were dissolved in acetonitrile (8 mL) at room temperature, after which the resulting solution was stirred at the same temperature for 18 hours. Solvent was removed from the reaction mixture under reduced pressure, after which water was poured into the resulting concentrate, and then an organic layer was extracted with dichloromethane, filtered via a plastic filter to remove a solid residue and an aqueous solution layer therefrom, and concentrated under reduced pressure. The resulting concentrate was purified via column chromatography (SiO₂, 12 g cartridge; methanol/dichloromethane=1%) and concentrated to obtain a title compound (0.790 g, 99.6%) as a white solid form.

### [Step 2] Synthesis of 1-(2,2-difluoropropyl)piperazine hydrochloride

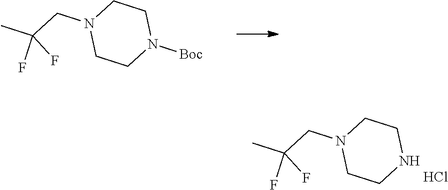 [SMILES: CC(F)(F)CN1CCN(C(=O)OC(C)(C)C)CC1 ; CC(F)(F)CN1CCNCC1 ; Cl]

Tert-butyl 4-(2,2-difluoropropyl)piperazin-1-carboxylate (0.790 g, 2.989 mmol) prepared in step 1 and hydrogen chloride (4.00 M solution in 1,4-dioxane, 5.978 mL, 23.910 mmol) were dissolved in dichloromethane (6 mL) at room temperature, after which the resulting solution was stirred at the same temperature for 18 hours. Solvent was removed from the reaction mixture under reduced pressure, after which an obtained product was used without an additional purification process (title compound, 0.599 g, 99.9%, white solid).

### [Step 3] Synthesis of tert-butyl (S)-2-(4-(2,2-difluoropropyl)piperazin-1-carbonyl)pyrrolidin-1-carboxylate

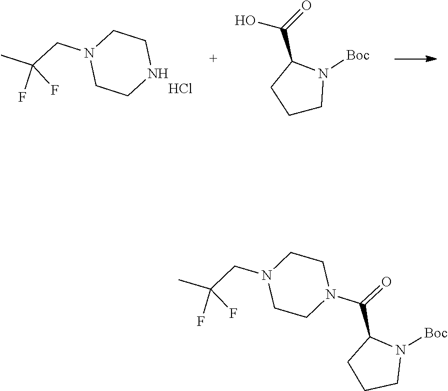 [SMILES: CC(F)(F)CN1CCNCC1 ; Cl ; CC(C)(C)OC(=O)N1CCC[C@H]1C(=O)O ; CC(F)(F)CN1CCN(C(=O)[C@@H]2CCCN2C(=O)OC(C)(C)C)CC1]

1-(2,2-Difluoropropyl)piperazine hydrochloride (0.201 g, 1.000 mmol) prepared in step 2, (tert-butoxycarbonyl)-L-proline (0.215 g, 1.000 mmol), triethylamine (0.348 mL, 2.500 mmol) and 2,4,6-tripropyl-1,3,5,2,4,6-trioxatriphosphinane 2,4,6-trioxide (T3P, 50.00% solution in EtOAc, 0.707 mL, 1.200 mmol) were dissolved in dichloromethane (4 mL) at room temperature, after which the resulting solution was stirred at the same temperature for 18 hours. Saturated aqueous solution of sodium hydrogen carbonate was poured into the reaction mixture, and then an organic layer was extracted with dichloromethane, filtered via a plastic filter to remove a solid residue and an aqueous solution layer therefrom, and concentrated under reduced pressure. The resulting concentrate was purified via column chromatography (SiO₂, 12 g cartridge; methanol/dichloromethane=1.5%) and concentrated to obtain a title compound (0.360 g, 99.6%) as a colorless oil form.

### [Step 4] Synthesis of (S)-1-(2,2-difluoropropyl)-4-prolylpiperazine hydrochloride

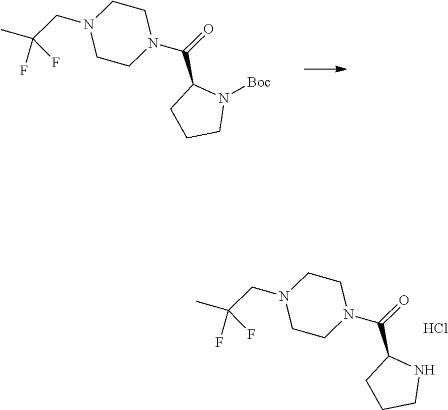 [SMILES: CC(F)(F)CN1CCN(C(=O)[C@@H]2CCCN2C(=O)OC(C)(C)C)CC1 ; CC(F)(F)CN1CCN(C(=O)[C@@H]2CCCN2)CC1 ; Cl]

Tert-butyl (S)-2-(4-(2,2-difluoropropyl)piperazin-1-carbonyl)pyrrolidin-1-carboxylate (0.360 g, 0.996 mmol) prepared in step 3 and hydrogen chloride (4.00 M solution in 1,4-dioxane, 1.494 mL, 5.976 mmol) were dissolved in dichloromethane (3 mL) at room temperature, after which the resulting solution was stirred at the same temperature for 18 hours. Solvent was removed from the reaction mixture under reduced pressure, after which an obtained product was used without an additional purification process (title compound, 0.227 g, 76.4%, white solid).

### [Step 5] Synthesis of (S)-(1-(5-amino-2-(furan-2-yl)-[1,2,4]triazolo[1,5-c]pyrimidin-7-yl)pyrrolidin-2-yl)(4-(2,2-difluoropropyl)piperazin-1-yl)methanone

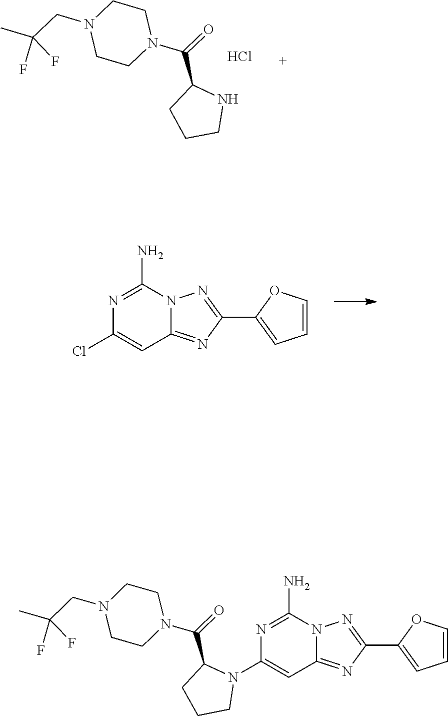 [SMILES: CC(F)(F)CN1CCN(C(=O)[C@@H]2CCCN2)CC1 ; Cl ; Nc1nc(Cl)cc2nc(-c3ccco3)nn12 ; CC(F)(F)CN1CCN(C(=O)[C@@H]2CCCN2c2cc3nc(-c4ccco4)nn3c(N)n2)CC1]

(S)-1-(2,2-Difluoropropyl)-4-prolylpiperazine hydrochloride (0.227 g, 0.761 mmol) prepared in step 4, 7-chloro-2-(furan-2-yl)-[1,2,4]triazolo[1,5-c]pyrimidin-5-amine (0.179 g, 0.761 mmol) and sodium hydrogen carbonate (0.192 g, 2.284 mmol) were dissolved in N,N-dimethylformamide (2 mL) at room temperature, after which the resulting solution was stirred at 100° C. for 18 hours to complete the reaction by lowering a temperature to room temperature. Water was poured into the reaction mixture, and then an organic layer was extracted with dichloromethane, filtered via a plastic filter to remove a solid residue and an aqueous solution layer therefrom, and concentrated under reduced pressure. The resulting concentrate was purified via column chromatography (SiO₂, 12 g cartridge; methanol/dichloromethane=0 to 5%) and concentrated to obtain a product, after which the obtained product was purified again via chromatography (SiO₂, 12 g cartridge; acetone/dichloromethane=10 to 100%) and concentrated to obtain a title compound (0.055 g, 15.8%) as a light yellow solid form.

¹H NMR (400 MHz, DMSO-d₆) δ7.86 (dd, J=1.7, 0.8 Hz, 1H), 7.46 (brs, 2H), 7.06 (dd, J=3.4, 0.6 Hz, 1H), 6.67 (dd, J=3.4, 1.8 Hz, 1H), 5.59 (brs, 1H), 4.96 (brs, 1H), 3.79-3.38 (m, 5H), 2.79 (t, J=14.1 Hz, 3H), 2.64-2.36 (m, 4H), 2.24 (s, 1H), 2.06-1.77 (m, 3H), 1.66 (t, J=19.1 Hz, 3H); LRMS (ES) m/z 461.5 (M⁺+1).

### Example 371: Synthesis of Compound 371, ((S)-1-(7-amino-2-(furan-2-yl)-[1,2,4]triazolo[1,5-a][1,3,5]triazin-5-yl)pyrrolidin-2-yl)((R)-4-(2,2-difluoroethyl)-2-methylpiperazin-1-yl)methanone

### [Step 1] Synthesis of tert-butyl (R)-4-(2,2-difluoroethyl)-2-methylpiperazin-1-carboxylate

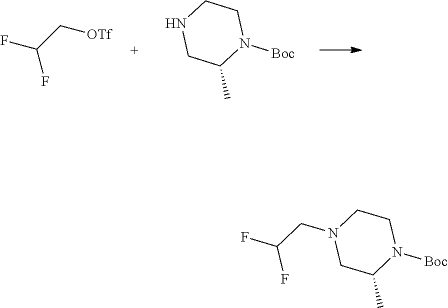 [SMILES: O=S(=O)(OCC(F)F)C(F)(F)F ; C[C@@H]1CNCCN1C(=O)OC(C)(C)C ; C[C@@H]1CN(CC(F)F)CCN1C(=O)OC(C)(C)C]

Tert-butyl (R)-2-methylpiperazin-1-carboxylate (0.507 g, 2.531 mmol), 2,2-difluoroethyl trifluoromethanesulfonate (0.542 g, 2.531 mmol) and potassium carbonate (1.050 g, 7.594 mmol) were dissolved in acetonitrile (10 mL) at room temperature, after which the resulting solution was stirred at the same temperature for 18 hours. The reaction mixture was filtered via a plastic filter to remove a solid therefrom, after which solvent was removed from the resulting filtrate without the solid under reduced pressure, and then an obtained product was used without an additional purification process (title compound, 0.668 g, 99.8%, transparent liquid).

### [Step 2] Synthesis of (R)-1-(2,2-difluoroethyl)-3-methylpiperazine hydrochloride

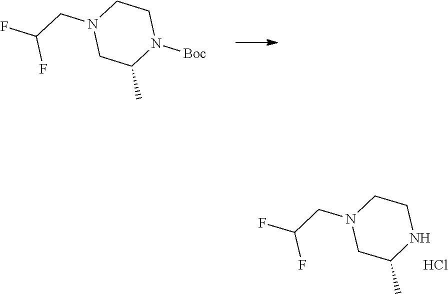 [SMILES: C[C@@H]1CN(CC(F)F)CCN1C(=O)OC(C)(C)C ; C[C@@H]1CN(CC(F)F)CCN1 ; Cl]

Tert-butyl (R)-4-(2,2-difluoroethyl)-2-methylpiperazin-1-carboxylate (0.668 g, 2.527 mmol) prepared in step 1 and hydrochloric acid (4.00 M solution in 1,4-dioxane, 2.527 mL, 10.109 mmol) were dissolved in dichloromethane (10 mL) at room temperature, after which the resulting solution was stirred at the same temperature for 18 hours. Solvent was removed from the reaction mixture under reduced pressure, after which an obtained product was used without an additional purification process (title compound, 0.507 g, 100.0%, white solid).

### [Step 3] Synthesis of tert-butyl (S)-2-((R)-4-(2,2-difluoroethyl)-2-methylpiperazin-1-carbonyl)pyrrolidin-1-carboxylate

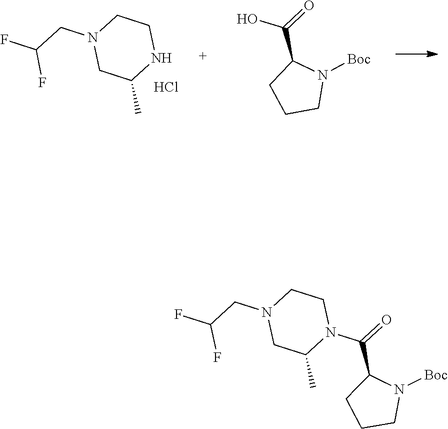 [SMILES: C[C@@H]1CN(CC(F)F)CCN1 ; Cl ; CC(C)(C)OC(=O)N1CCC[C@H]1C(=O)O ; C[C@@H]1CN(CC(F)F)CCN1C(=O)[C@@H]1CCCN1C(=O)OC(C)(C)C]

(R)-1-(2,2-Difluoroethyl)-3-methylpiperazine hydrochloride (0.507 g, 2.527 mmol) prepared in step 2, (tert-butoxycarbonyl)-L-proline (0.544 g, 2.527 mmol), 2,4,6-tripropyl-1,3,5,2,4,6-trioxatriphosphinane 2,4,6-trioxide (T3P, 50.00% solution in EtOAc, 2.232 mL, 3.790 mmol) and N,N-diisopropylethylamine (1.320 mL, 7.580 mmol) were dissolved in dichloromethane (10 mL) at room temperature, after which the resulting solution was stirred at the same temperature. Aqueous solution of N-sodium hydrogen carbonate was poured into the reaction mixture and an organic layer was extracted with dichloromethane. The organic layer was washed with saturated aqueous solution of sodium chloride, dehydrated with anhydrous sodium sulfate, filtered, and concentrated under reduced pressure. An obtained product was used without an additional purification process (title compound, 1.028 g, 98.9%, light yellow liquid).

### [Step 4] Synthesis of (R)-1-(L-prolyl)-4-(2,2-difluoroethyl)-2-methylpiperazine hydrochloride

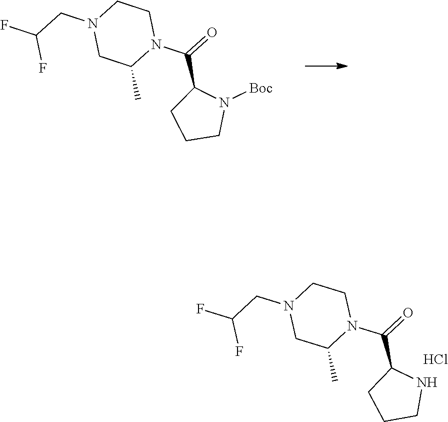 [SMILES: C[C@@H]1CN(CC(F)F)CCN1C(=O)[C@@H]1CCCN1C(=O)OC(C)(C)C ; C[C@@H]1CN(CC(F)F)CCN1C(=O)[C@@H]1CCCN1 ; Cl]

Tert-butyl (S)-2-((R)-4-(2,2-difluoroethyl)-2-methylpiperazin-1-carbonyl)pyrrolidin-1-carboxylate (0.903 g, 2.498 mmol) prepared in step 3 and hydrochloric acid (4.00 M solution in 1,4-dioxane, 2.498 mL, 9.994 mmol) were dissolved in dichloromethane (10 mL) at room temperature, after which the resulting solution was stirred at the same temperature for 18 hours. Solvent was removed from the reaction mixture under reduced pressure, after which an obtained product was used without an additional purification process (title compound, 0.743 g, 99.9%, white solid).

### [Step 5] ((S)-1-(7-amino-2-(furan-2-yl)-[1,2,4]triazolo[1,5-a][1,3,5]triazin-5-yl)pyrrolidin-2-yl)((R)-4-(2,2-difluoroethyl)-2-methylpiperazin-1-yl)methanone

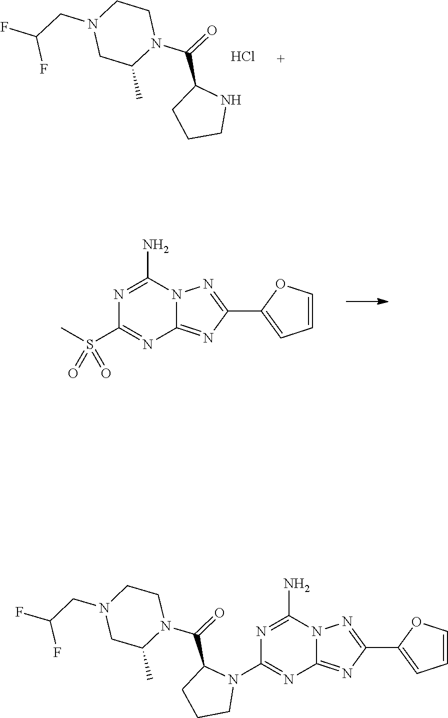 [SMILES: C[C@@H]1CN(CC(F)F)CCN1C(=O)[C@@H]1CCCN1 ; Cl ; CS(=O)(=O)c1nc(N)n2nc(-c3ccco3)nc2n1 ; C[C@@H]1CN(CC(F)F)CCN1C(=O)[C@@H]1CCCN1c1nc(N)n2nc(-c3ccco3)nc2n1]

(R)-1-(L-Prolyl)-4-(2,2-difluoroethyl)-2-methylpiperazine hydrochloride (0.743 g, 2.495 mmol) prepared in step 4, 2-(furan-2-yl)-5-(methylsulfonyl)-[1,2,4]triazolo[1,5-a][1,3,5]triazin-7-amine (0.699 g, 2.495 mmol) and sodium hydrogen carbonate (0.629 g, 7.486 mmol) were dissolved in acetonitrile (15 mL) at room temperature, after which the resulting solution was stirred at 75° C. for 18 hours to complete the reaction by lowering a temperature to room temperature. The reaction mixture was filtered via a celite pad to remove a solid therefrom, after which solvent was removed from the resulting filtrate without the solid under reduced pressure. Then, the resulting concentrate was purified via column chromatography (SiO₂, 12 g cartridge; dichloromethane/methanol=0 to 30%) and concentrated to obtain a title compound (0.509 g, 44.2%) as a white solid form.

¹H NMR (400 MHz, DMSO-d₆) δ 8.56-7.93 (m, 2H), 7.90-7.83 (m, 1H), 7.06 (dd, J=8.5, 3.3 Hz, 1H), 6.67 (ddd, J=6.4, 3.3, 1.6 Hz, 1H), 6.39-5.95 (m, 1H), 4.96 (dddd, J=35.1, 26.4, 8.5, 2.6 Hz, 1H), 4.33 (d, J=100.5 Hz, 1H), 4.15-3.76 (m, 1H), 3.70-3.53 (m, 2H), 2.96-2.64 (m, 5H), 2.48-2.00 (m, 3H), 1.98-1.69 (m, 3H), 1.49 (dd, J=15.8, 6.6 Hz, 2H), 1.13 (d, J=6.8 Hz, 1H). LRMS (ES) m/z 462.4 (M⁺+1).

### Example 258: Synthesis of Compound 258

Example compound 258 was synthesized through substantially the same synthesis method as a synthesis method of example compound 223 except for using 2-(furan-2-yl)-5-(methylsulfonyl)-3a,7a-dihydrothiazolo[5,4-d]pyrimidin-7-amine instead of 2-(furan-2-yl)-5-(methylsulfonyl)-[1,2,4]triazolo[1,5-a][1,3,5]triazin-7-amine in the synthesis method of example compound 223.

### Example 259: Synthesis of Compound 259

Example compound 259 was synthesized through substantially the same synthesis method as a synthesis method of example compound 224 except for using 2-(furan-2-yl)-5-(methylsulfonyl)-3a,7a-dihydrothiazolo[5,4-d]pyrimidin-7-amine instead of 2-(furan-2-yl)-5-(methylsulfonyl)-[1,2,4]triazolo[1,5-a][1,3,5]triazin-7-amine in the synthesis method of example compound 224.

### Example 260: Synthesis of Compound 260

Example compound 260 was synthesized through substantially the same synthesis method as a synthesis method of example compound 223 except for using 1,1,2,2-tetrafluoro-3-((trifluoromethyl)sulfonyl)propane instead of 2,2-difluoroethyl trifluoromethanesulfonate and using 2-(furan-2-yl)-5-(methylsulfonyl)-3a,7a-dihydrothiazolo[5,4-d]pyrimidin-7-amine instead of 2-(furan-2-yl)-5-(methylsulfonyl)-[1,2,4]triazolo[1,5-a][1,3,5]triazin-7-amine in the synthesis method of example compound 223.

### Example 261: Synthesis of Compound 261

Example compound 261 was synthesized through substantially the same synthesis method as a synthesis method of example compound 223 except for using 1,1,1,2,2-pentafluoro-3-((trifluoromethyl)sulfonyl)propane instead of 2,2-difluoroethyl trifluoromethanesulfonate and using 2-(furan-2-yl)-5-(methylsulfonyl)-3a,7a-dihydrothiazolo[5,4-d]pyrimidin-7-amine instead of 2-(furan-2-yl)-5-(methylsulfonyl)-[1,2,4]triazolo[1,5-a][1,3,5]triazin-7-amine in the synthesis method of example compound 223.

### Example 269: Synthesis of Compound 269

Example compound 269 was synthesized through substantially the same synthesis method as a synthesis method of example compound 223 except for using 2-(furan-2-yl)-5-(methylsulfonyl)-3a,7a-dihydrooxazolo[5,4-d]pyrimidin-7-amine instead of 2-(furan-2-yl)-5-(methylsulfonyl)-[1,2,4]triazolo[1,5-a][1,3,5]triazin-7-amine in the synthesis method of example compound 223.

### Example 270: Synthesis of Compound 270

Example compound 270 was synthesized through substantially the same synthesis method as a synthesis method of example compound 223 except for using 2,2-difluoro-1-((trifluoromethyl)sulfonyl)butane instead of 2,2-difluoroethyl trifluoromethanesulfonate and using 2-(furan-2-yl)-5-(methylsulfonyl)-3a,7a-dihydrooxazolo[5,4-d]pyrimidin-7-amine instead of 2-(furan-2-yl)-5-(methylsulfonyl)-[1,2,4]triazolo[1,5-a][1,3,5]triazin-7-amine in the synthesis method of example compound 223.

### Example 271: Synthesis of Compound 271

Example compound 271 was synthesized through substantially the same synthesis method as a synthesis method of example compound 223 except for using 1,1,2,2-tetrafluoro-3-((trifluoromethyl)sulfonyl)propane instead of 2,2-difluoroethyl trifluoromethanesulfonate and using 2-(furan-2-yl)-5-(methylsulfonyl)-3a,7a-dihydrooxazolo[5,4-d]pyrimidin-7-amine instead of 2-(furan-2-yl)-5-(methylsulfonyl)-[1,2,4]triazolo[1,5-a][1,3,5]triazin-7-amine in the synthesis method of example compound 223.

### Example 293: Synthesis of Compound 293

Example compound 293 was synthesized through substantially the same synthesis method as a synthesis method of example compound 223 except for using 2-(furan-2-yl)-7-(methylsulfonyl)-[1,2,4]triazolo[1,5-c]pyrimidin-5-amine instead of 2-(furan-2-yl)-5-(methylsulfonyl)-[1,2,4]triazolo[1,5-a][1,3,5]triazin-7-amine in the synthesis method of example compound 223.

### Example 295: Synthesis of Compound 295

Example compound 295 was synthesized through substantially the same synthesis method as a synthesis method of example compound 223 except for using 2,2-difluoro-1-((trifluoromethyl)sulfonyl)butane instead of 2,2-difluoroethyl trifluoromethanesulfonate and using 2-(furan-2-yl)-7-(methylsulfonyl)-[1,2,4]triazolo[1,5-c]pyrimidin-5-amine instead of 2-(furan-2-yl)-5-(methylsulfonyl)-[1,2,4]triazolo[1,5-a][1,3,5]triazin-7-amine in the synthesis method of example compound 223.

### Example 296: Synthesis of Compound 296

Example compound 296 was synthesized through substantially the same synthesis method as a synthesis method of example compound 223 except for using 1,1,1,2,2-pentafluoro-3-((trifluoromethyl)sulfonyl)propane instead of 2,2-difluoroethyl trifluoromethanesulfonate and using 2-(furan-2-yl)-7-(methylsulfonyl)-[1,2,4]triazolo[1,5-c]pyrimidin-5-amine instead of 2-(furan-2-yl)-5-(methylsulfonyl)-[1,2,4]triazolo[1,5-a][1,3,5]triazin-7-amine in the synthesis method of example compound 223.

### Examples 316, 317, 318 and 319

Example compounds 316, 317, 318 and 319 were each synthesized through substantially the same synthesis method as each synthesis method except for using (tert-butoxycarbonyl)-L-alanine instead of (tert-butoxycarbonyl)-L-proline and using 2-(furan-2-yl)-5-(methylsulfonyl)-[1,2,4]triazolo[1,5-a][1,3,5]triazin-7-amine instead of 2-(furan-2-yl)-5-(methylsulfonyl)-3a,7a-dihydrothiazolo[5,4-d]pyrimidin-7-amine in each synthesis method of example compounds 258, 259, 260 and 261.

### Example 320: Synthesis of Compound 320

Example compound 320 was synthesized through substantially the same synthesis method as a synthesis method of example compound 223 except for using 1,1,1,2,2,3,3-heptafluoro-4-((trifluoromethyl)sulfonyl)butane instead of 2,2-difluoroethyl trifluoromethanesulfonate and using (tert-butoxycarbonyl)-L-alanine instead of (tert-butoxycarbonyl)-L-proline in the synthesis method of example compound 223.

### Examples 323, 324, 325 and 326

Example compounds 323, 324, 325 and 326 were each synthesized through substantially the same synthesis method as each synthesis method except for using (S)-1-(tert-butoxycarbonyl)azetidine-2-carboxylic acid instead of (tert-butoxycarbonyl)-L-alanine and using tert-butyl [4,4′-bipiperidine]-1-carboxylate instead of tert-butyl piperazin-1-carboxylate in the synthesis method of example compounds 316, 317, 318 and 319.

### Examples 334 and 335

Example compounds 334 and 335 were synthesized through substantially the same synthesis method as each synthesis method except for using tert-butyl [4,4′-bipiperidine]-1-carboxylate instead of tert-butyl piperazin-1-carboxylate in each synthesis method of example compounds 316 and 317.

### Examples 252, 253, 255, 256 and 257

Example compounds 252, 253, 255, 256 and 257 were each synthesized through substantially the same synthesis method as each synthesis method except for using (S)-1-(tert-butoxycarbonyl)azetidin-2-carboxylic acid instead of (tert-butoxycarbonyl)-L-alanine in the synthesis method of example compounds 316, 317, 318 and 319.

### Example 254: Synthesis of Compound 254

Example compound 254 was synthesized through substantially the same synthesis method except for using (S)-1-(tert-butoxycarbonyl)azetidin-2-carboxylic acid instead of tert-butoxycarbonyl-D-proline in the synthesis method of example compound 225.

### Examples 204, 205, 206, 225, 226, 227, 228, 278, 362, 363, 364, 365, 287, 288, 289, 337, 341, 342, 343 and 345

Example compounds 204, 205, 206, 225, 226, 227, 228, 278, 362, 363, 364, 365, 287, 288, 289, 337, 341, 342, 343 and 345 were each synthesized through substantially the same synthesis method as a synthesis method of example compound 224 except for using starting material 1 of the table below instead of tert-butyl piperazin-1-carboxylate of step 1 in the synthesis method of example compound 224 and using starting material 2 of the table below instead of difluoropropyl trifluoromethanesulfonate.

TABLE 32

| Example | Starting material 1 | Starting material 2 |
|---|---|---|
| No. | of step 1 | of step 1 |
| 204 | 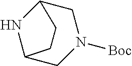 [SMILES: CC(C)(C)OC(=O)N1CC2CCC(C1)N2] | 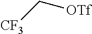 [SMILES: O=S(=O)(OCC(F)(F)F)C(F)(F)F] |
| 205 | 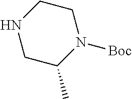 | 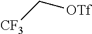 [SMILES: O=S(=O)(OCC(F)(F)F)C(F)(F)F] |
| 206 | 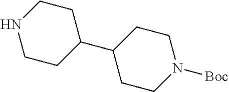 [SMILES: CC(C)(C)OC(=O)N1CCC(C2CCNCC2)CC1] | 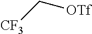 [SMILES: O=S(=O)(OCC(F)(F)F)C(F)(F)F] |
| 225 | 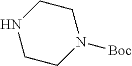 [SMILES: CC(C)(C)OC(=O)N1CCNCC1] | 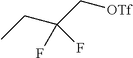 |
| 226 | 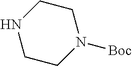 [SMILES: CC(C)(C)OC(=O)N1CCNCC1] | 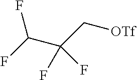 [SMILES: O=S(=O)(OCC(F)(F)C(F)F)C(F)(F)F] |
| 227 | 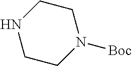 [SMILES: CC(C)(C)OC(=O)N1CCNCC1] | 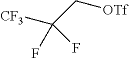 [SMILES: O=S(=O)(OCC(F)(F)C(F)(F)F)C(F)(F)F] |
| 228 | 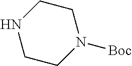 [SMILES: CC(C)(C)OC(=O)N1CCNCC1] | 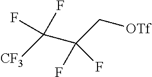 [SMILES: O=S(=O)(OCC(F)(F)C(F)(F)C(F)(F)F)C(F)(F)F] |
| 278 | 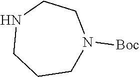 [SMILES: CC(C)(C)OC(=O)N1CCCNCC1] | 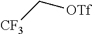 [SMILES: O=S(=O)(OCC(F)(F)F)C(F)(F)F] |
| 362 | 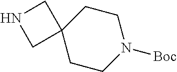 [SMILES: CC(C)(C)OC(=O)N1CCC2(CC1)CNC2] | 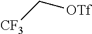 [SMILES: O=S(=O)(OCC(F)(F)F)C(F)(F)F] |
| 363 | 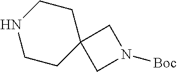 [SMILES: CC(C)(C)OC(=O)N1CC2(CCNCC2)C1] | 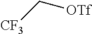 [SMILES: O=S(=O)(OCC(F)(F)F)C(F)(F)F] |
| 364 | 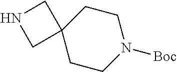 [SMILES: CC(C)(C)OC(=O)N1CCC2(CC1)CNC2] | 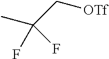 |
| 365 | 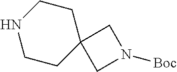 [SMILES: CC(C)(C)OC(=O)N1CC2(CCNCC2)C1] | 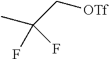 |
| 287 | 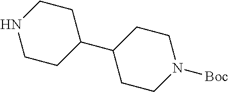 [SMILES: CC(C)(C)OC(=O)N1CCC(C2CCNCC2)CC1] | 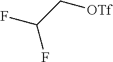 [SMILES: O=S(=O)(OCC(F)F)C(F)(F)F] |
| 288 | 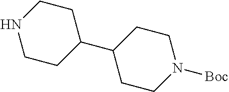 [SMILES: CC(C)(C)OC(=O)N1CCC(C2CCNCC2)CC1] | 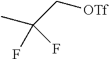 |
| 289 | 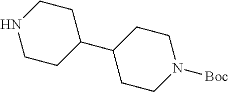 [SMILES: CC(C)(C)OC(=O)N1CCC(C2CCNCC2)CC1] | 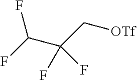 [SMILES: O=S(=O)(OCC(F)(F)C(F)F)C(F)(F)F] |
| 337 | 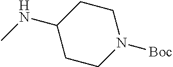 | 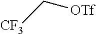 [SMILES: O=S(=O)(OCC(F)(F)F)C(F)(F)F] |
| 341 | 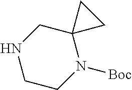 [SMILES: CC(C)(C)OC(=O)N1CCNCC12CC2] | 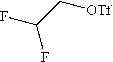 [SMILES: O=S(=O)(OCC(F)F)C(F)(F)F] |
| 342 | 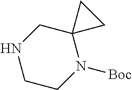 [SMILES: CC(C)(C)OC(=O)N1CCNCC12CC2] | 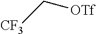 [SMILES: O=S(=O)(OCC(F)(F)F)C(F)(F)F] |
| 343 | 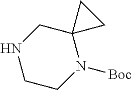 [SMILES: CC(C)(C)OC(=O)N1CCNCC12CC2] | 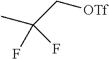 |
| 345 | 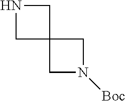 [SMILES: CC(C)(C)OC(=O)N1CC2(CNC2)C1] | 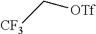 [SMILES: O=S(=O)(OCC(F)(F)F)C(F)(F)F] |
| 372 | 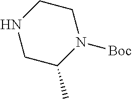 | 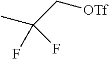 |

### Example 196: Synthesis of Compound 196

Example compound 196 was synthesized through substantially the same synthesis method as a synthesis method of example compound 205 except for using 2-(methylfuran-2-yl)-5-(methylsulfonyl)-[1,2,4]triazolo[1,5-a][1,3,5]triazin-7-amine instead of 2-(furan-2-yl)-5-(methylsulfonyl)-[1,2,4]triazolo[1,5-a][1,3,5]triazin-7-amine in the synthesis method of example compound 205.

### Example 197: Synthesis of Compound 197

Example compound 197 was synthesized through substantially the same synthesis method as a synthesis method of example compound 196 except for using tert-butyl [4,4′-bipiperidine]-1-carboxylate instead of tert-butyl (R)-2-methylpiperazin-1-carboxylate.

### Example 279: Synthesis of Compound 279

Example compound 279 was synthesized through substantially the same synthesis method as a synthesis method of example compound 223 except for using 1,1,2,2-tetrafluoro-3-((trifluoromethyl)sulfonyl)propane instead of 2,2-difluoroethyl trifluoromethanesulfonate and using (S)-1-(tert-butoxycarbonyl)piperidin-2-carboxylic acid instead of (tert-butoxycarbonyl)-L-proline.

### Example 280: Synthesis of Compound 280

Example compound 280 was synthesized through substantially the same synthesis method as a synthesis method of example compound 223 except for using 1,1,1,2,2-pentafluoro-3-((trifluoromethyl)sulfonyl)propane instead of 2,2-difluoroethyl trifluoromethanesulfonate and using (S)-1-(tert-butoxycarbonyl)piperidin-2-carboxylic acid instead of (tert-butoxycarbonyl)-L-proline.

### Example 297: Synthesis of Compound 297

Example compound 297 was synthesized through substantially the same synthesis method as a synthesis method of example compound 223 except for using 2-(methylfuran-2-yl)-5-(methylsulfonyl)-[1,2,4]triazolo[1,5-a][1,3,5]triazin-7-amine instead of 2-(furan-2-yl)-5-(methylsulfonyl)-[1,2,4]triazolo[1,5-a][1,3,5]triazin-7-amine.

### Example 298: Synthesis of Compound 298

Example compound 298 was synthesized through substantially the same synthesis method as a synthesis method of example compound 223 except for using 1,1,2,2-tetrafluoro-3-((trifluoromethyl)sulfonyl)propane instead of 2,2-difluoroethyl trifluoromethanesulfonate and using 2-(methylfuran-2-yl)-5-(methylsulfonyl)-[1,2,4]triazolo[1,5-a][1,3,5]triazin-7-amine instead of 2-(furan-2-yl)-5-(methylsulfonyl)-[1,2,4]triazolo[1,5-a][1,3,5]triazin-7-amine.

### Example 299: Synthesis of Compound 299

Example compound 299 was synthesized through substantially the same synthesis method as a synthesis method of example compound 223 except for using 1,1,1,2,2-pentafluoro-3-((trifluoromethyl)sulfonyl)propane instead of 2,2-difluoroethyl trifluoromethanesulfonate and using 2-(methylfuran-2-yl)-5-(methylsulfonyl)-[1,2,4]triazolo[1,5-a][1,3,5]triazin-7-amine instead of 2-(furan-2-yl)-5-(methylsulfonyl)-[1,2,4]triazolo[1,5-a][1,3,5]triazin-7-amine.

### Example 311: Synthesis of Compound 311

Example compound 311 was synthesized through substantially the same synthesis method as a synthesis method of example compound 223 except for using 2-(methylfuran-2-yl)-5-(methylsulfonyl)-[1,2,4]triazolo[1,5-a][1,3,5]triazin-7-amine instead of 2-(furan-2-yl)-5-(methylsulfonyl)-[1,2,4]triazolo[1,5-a][1,3,5]triazin-7-amine.

### Example 312: Synthesis of Compound 312

Example compound 312 was synthesized through substantially the same synthesis method as a synthesis method of example compound 298 except for using (S)-1-(tert-butoxycarbonyl)azetidin-2-carboxylic acid instead of tert-butoxycarbonyl-D-proline.

### Example 313: Synthesis of Compound 313

Example compound 313 was synthesized through substantially the same synthesis method as a synthesis method of example compound 299 except for using (S)-1-(tert-butoxycarbonyl)azetidin-2-carboxylic acid instead of tert-butoxycarbonyl-D-proline.

### Example 314: Synthesis of Compound 314

Example compound 314 was synthesized through substantially the same synthesis method as a synthesis method of example compound 311 except for using (S)-1-(tert-butoxycarbonyl)azetidin-2-carboxylic acid instead of tert-butoxycarbonyl-D-proline.

### Examples 354, 355, 356, 357, 358, 359 and 360

Example compounds 354, 355, 356, 357, 358, 359 and 360 were each prepared through substantially the same synthesis method as a synthesis method of example compound 223 except for using starting material 1 of the table below instead of 2,2-difluoroethyl trifluoromethanesulfonate of step 1 and using Boc-protected amino acids of the table below instead of (tert-butoxycarbonyl)-L-proline.

TABLE 33

| Example | Starting material 1 of | Amino |
|---|---|---|
| No. | step 1 | acid |
| 354 | 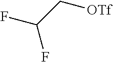 [SMILES: O=S(=O)(OCC(F)F)C(F)(F)F] | 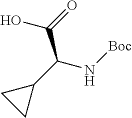 [SMILES: CC(C)(C)OC(=O)N[C@H](C(=O)O)C1CC1] |
| 355 | 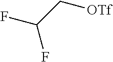 [SMILES: O=S(=O)(OCC(F)F)C(F)(F)F] | 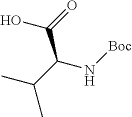 |
| 356 | 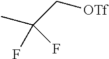 | 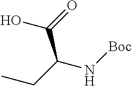 |
| 357 | 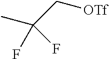 | 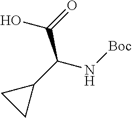 [SMILES: CC(C)(C)OC(=O)N[C@H](C(=O)O)C1CC1] |
| 358 | 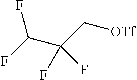 [SMILES: O=S(=O)(OCC(F)(F)C(F)F)C(F)(F)F] | 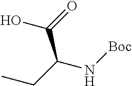 |
| 359 | 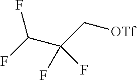 [SMILES: O=S(=O)(OCC(F)(F)C(F)F)C(F)(F)F] | 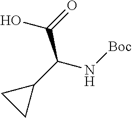 [SMILES: CC(C)(C)OC(=O)N[C@H](C(=O)O)C1CC1] |
| 360 | 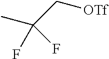 | 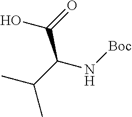 |

### Examples 376, 377, 378, 379, 393 and 394

Example compounds 376, 377, 378, 379, 393 and 394 were each synthesized through substantially the same synthesis method as a synthesis method of example compound 223 except for using 2,2-difluoroethyl trifluoromethanesulfonate of step 1 or starting material 1 of the table below, using (tert-butoxycarbonyl)-L-proline or Boc-protected amino acid of the table below, and using 2-(furan-2-yl)-7-(methylsulfonyl)-[1,2,4]triazolo[1,5-c]pyrimidin-5-amine instead of 2-(furan-2-yl)-5-(methylsulfonyl)-[1,2,4]triazolo[1,5-a][1,3,5]triazin-7-amine.

TABLE 34

| Example | Starting material | Boc-protected |
|---|---|---|
| No. | 1 of step 1 | amino acid |
| 376 | 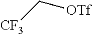 [SMILES: O=S(=O)(OCC(F)(F)F)C(F)(F)F] | 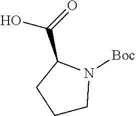 [SMILES: CC(C)(C)OC(=O)N1CCC[C@H]1C(=O)O] |
| 377 | 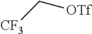 [SMILES: O=S(=O)(OCC(F)(F)F)C(F)(F)F] | 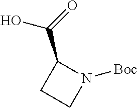 [SMILES: CC(C)(C)OC(=O)N1CC[C@H]1C(=O)O] |
| 378 | 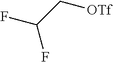 [SMILES: O=S(=O)(OCC(F)F)C(F)(F)F] | 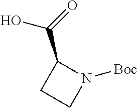 [SMILES: CC(C)(C)OC(=O)N1CC[C@H]1C(=O)O] |
| 379 | 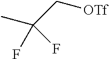 | 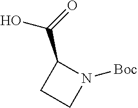 [SMILES: CC(C)(C)OC(=O)N1CC[C@H]1C(=O)O] |
| 393 | 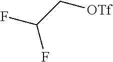 [SMILES: O=S(=O)(OCC(F)F)C(F)(F)F] | 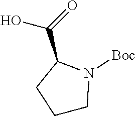 [SMILES: CC(C)(C)OC(=O)N1CCC[C@H]1C(=O)O] |
| 394 | 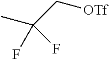 | 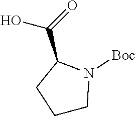 [SMILES: CC(C)(C)OC(=O)N1CCC[C@H]1C(=O)O] |

### Example 366: Synthesis of Compound 366

Example compound 366 was synthesized through substantially the same synthesis method as a synthesis method of example compound 365 except for using (tert-butoxycarbonyl)-L-alanine instead of tert-butoxycarbonyl)-L-proline.

### Example 396: Synthesis of Compound 396

Example compound 396 was synthesized through substantially the same synthesis method as a synthesis method of example compound 223 except for using N-(tert-butoxycarbonyl)-O-methyl-L-serine instead of (tert-butoxycarbonyl)-L-proline.

### Example 397: Synthesis of Compound 397

Example compound 397 was synthesized through substantially the same synthesis method as a synthesis method of example compound 224 except for using N-(tert-butoxycarbonyl)-O-methyl-L-serine instead of (tert-butoxycarbonyl)-L-proline.

Analysis data of each of the compounds prepared as described above are shown in the table below.

TABLE 35

| Example | | |
|---|---|---|
| No. | Compound Names | Analysis Data |
| 258 | (S)-(1-(7-amino-2-(furan-2- | ¹H NMR (400 MHz, DMSO-d₆) δ 7.90 (dd, J = 1.7, |
| | yl)thiazolo[5,4-d]pyrimidin-5- | 0.6 Hz, 1H), 7.39-6.88 (m, 3H), 6.72 (dd, J = 3.5, |
| | yl)pyrrolidin-2-yl)(4-(2,2- | 1.8 Hz, 1H), 6.19 (t, J = 55.3 Hz, 1H), 5.05-4.90 |
| | difluoroethyl)piperazin-1- | (m, 1H), 3.88-3.43 (m, 5H), 3.29-3.18 (m, 1H), |
| | yl)methanone | 2.87-2.55 (m, 5H), 2.45-2.34 (m, 1H), 2.30- |
| | | 2.14 (m, 1H), 2.02-1.86 (m, 2H), 1.85-1.72 (m, |
| | | 1H); LRMS (ES) m/z 464.3 (M⁺ + 1). |
| 259 | (S)-(1-(7-amino-2-(furan-2- | ¹H NMR (400 MHz, DMSO-d₆) δ 1H NMR (400 |
| | yl)thiazolo[5,4-d]pyrimidin-5- | MHz, DMSO-d6) 8 7.90 (dd, J = 1.8, 0.7 Hz, 1H), |
| | yl)pyrrolidin-2-yl)(4-(2,2- | 7.33-6.89 (m, 3H), 6.72 (dd, J = 3.5, 1.8 Hz, 1H), |
| | difluoropropyl)piperazin-1- | 5.03-4.88 (m, 1H), 3.87-3.44 (m, 5H), 3.32- |
| | yl)methanone | 3.20 (m, 1H), 2.86-2.55 (m, 5H), 2.45-2.36 (m, |
| | | 1H), 2.29-2.13 (m, 1H), 2.02-1.86 (m, 2H), 1.84- |
| | | 1.75 (m, 1H), 1.67 (t, J = 19.1 Hz, 3H); LRMS (ES) |
| | | m/z 478.3 (M⁺ + 1). |
| 260 | (S)-(1-(7-amino-2-(furan-2- | ¹H NMR (400 MHz, DMSO-d₆) δ 7.90 (dd, J = 1.7, |
| | yl)thiazolo[5,4-d]pyrimidin-5- | 0.6 Hz, 1H), 7.40-6.85 (m, 3H), 6.75-6.38 (m, |
| | yl)pyrrolidin-2-yl)(4-(2,2,3,3- | 2H), 5.06-4.88 (m, 1H), 3.90-3.45 (m, 5H), 3.31- |
| | tetrafluoropropyl)piperazin-1- | 3.21 (m, 1H), 3.07 (t, J = 15.1 Hz, 2H), 2.87-2.54 |
| | yl)methanone | (m, 4H), 2.29-2.13 (m, 1H), 2.02-1.85 (m, 2H), |
| | | 1.84-1.71 (m, 1H); LRMS (ES) m/z 514.2 (M⁺ + 1). |
| 261 | (S)-(1-(7-amino-2-(furan-2- | ¹H NMR (400 MHz, DMSO-d₆) δ 7.90 (dd, J = 1.7, |
| | yl)thiazolo[5,4-d]pyrimidin-5- | 0.6 Hz, 1H), 7.41-6.88 (m, 3H), 6.72 (dd, J = 3.5, |
| | yl)pyrrolidin-2-yl)(4- | 1.8 Hz, 1H), 5.05-4.89 (m, 1H), 3.93-3.44 (m, |
| | (2,2,3,3,3- | 5H), 3.30-3.18 (m, 3H), 2.91-2.56 (m, 4H), 2.28- |
| | pentafluoropropyl)piperazin-1- | 2.15 (m, 1H), 2.04-1.86 (m, 2H), 1.84-1.70 (m, |
| | yl)methanone | 1H); LRMS (ES) m/z 532.3 (M⁺ + 1). |
| 269 | (S)-(1-(7-amino-2-(furan-2- | ¹H NMR (400 MHz, DMSO-d₆) δ 7.95 (s, 1H), 7.46- |
| | yl)oxazolo[5,4-d]pyrimidin-5- | 6.89 (m, 3H), 6.75 (dd, J = 3.5, 1.8 Hz, 1H), 6.38- |
| | yl)pyrrolidin-2-yl)(4-(2,2- | 6.00 (m, 1H), 4.95 (dd, J = 8.6, 3.1 Hz, 1H), 3.78- |
| | difluoroethyl)piperazin-1- | 3.36 (m, 6H), 2.90-2.72 (m, 3H), 2.64-2.53 |
| | yl)methanone | (m, 3H), 2.31-2.15 (m, 1H), 1.99-1.87 (m, 2H), |
| | | 1.87-1.70 (m, 1H); LRMS (ES) m/z 448.4 (M⁺ + |
| | | 1). |
| 270 | (S)-(1-(7-amino-2-(furan-2- | ¹H NMR (400 MHz, DMSO-d₆) δ 7.95 (s, 1H), 7.49- |
| | yl)oxazolo[5,4-d]pyrimidin-5- | 6.87 (m, 3H), 6.75 (dd, J = 3.5, 1.8 Hz, 1H), 4.94 |
| | yl)pyrrolidin-2-yl)(4-(2,2- | (dd, J = 8.5, 3.1 Hz, 1H), 3.75-3.38 (m, 6H), 2.90- |
| | difluorobutyl)piperazin-1- | 2.71 (m, 3H), 2.66-2.55 (m, 3H), 2.32-2.14 (m, |
| | yl)methanone | 1H), 2.07-1.87 (m, 4H), 1.86-1.69 (m, 1H), 0.97 |
| | | (t, J = 7.5 Hz, 3H); LRMS (ES) m/z 476.4 (M⁺ + 1). |
| 271 | (S)-(1-(7-amino-2-(furan-2- | ¹H NMR (400 MHz, DMSO-d₆) δ 7.98-7.91 (m, |
| | yl)oxazolo[5,4-d]pyrimidin-5- | 1H), 7.46-6.90 (m, 3H), 6.75 (dd, J = 3.5, 1.7 Hz, |
| | yl)pyrrolidin-2-yl)(4-(2,2,3,3- | 1H), 6.72-6.38 (m, 1H), 4.94 (dd, J = 8.5, 3.1 Hz, |
| | tetrafluoropropyl)piperazin-1- | 1H), 3.77-3.35 (m, 6H), 3.07 (t, J = 15.5 Hz, 2H), |
| | yl)methanone | 2.81 (s, 1H), 2.67-2.53 (m, 3H), 2.30-2.13 (m, |
| | | 1H), 2.01-1.87 (m, 2H), 1.85-1.73 (m, 1H); LRMS |
| | | (ES) m/z 498.4 (M⁺ + 1). |
| 293 | (S)-(1-(5-amino-2-(furan-2-yl)- | ¹H NMR (400 MHz, DMSO-d₆) δ 7.86 (dd, J = 1.7, |
| | [1,2,4]triazolo[1,5-c]pyrimidin- | 0.8 Hz, 1H), 7.46 (brs, 2H), 7.06 (dd, J = 3.4, 0.6 |
| | 7-yl)pyrrolidin-2-yl)(4-(2,2- | Hz, 1H), 6.67 (dd, J = 3.4, 1.8 Hz, 1H), 6.18 (tt, J = |
| | difluoroethyl)piperazin-1- | 55.7, 4.3 Hz, 1H), 5.59 (brs, 1H), 4.96 (brs, 1H), 3.78- |
| | yl)methanone | 3.38 (m, 5H), 2.81 (td, J = 15.7, 4.3 Hz, 3H), 2.63- |
| | | 2.38 (m, 4H), 2.31-2.17 (m, 1H), 2.04-1.73 (m, |
| | | 3H); LRMS (ES) m/z 447.4 (M⁺ + 1). |
| 295 | (S)-(1-(5-amino-2-(furan-2-yl)- | ¹H NMR (400 MHz, DMSO-d₆) δ 7.86 (dd, J = 1.7, |
| | [1,2,4]triazolo[1,5-c]pyrimidin- | 0.8 Hz, 1H), 7.47 (brs, 2H), 7.06 (dd, J = 3.4, 0.7 |
| | 7-yl)pyrrolidin-2-yl)(4-(2,2- | Hz, 1H), 6.67 (dd, J = 3.4, 1.8 Hz, 1H), 5.59 (brs, |
| | difluorobutyl)piperazin-1- | 1H), 4.96 (brs, 1H), 3.78-3.38 (m, 5H), 2.79 (t, J = |
| | yl)methanone | 14.3 Hz, 3H), 2.62-2.38 (m, 4H), 2.30-2.15 (m, |
| | | 1H), 2.05-1.88 (m, 4H), 1.88-1.71 (m, 1H), 0.97 |
| | | (t, J = 7.5 Hz, 3H); LRMS (ES) m/z 475.4 (M⁺ + 1). |
| 296 | (S)-(1-(5-amino-2-(furan-2-yl)- | ¹H NMR (400 MHz, DMSO-d₆) δ 7.92-7.82 (m, |
| | [1,2,4]triazolo[1,5-c]pyrimidin- | 1H), 7.47 (brs, 2H), 7.14-7.00 (m, 1H), 6.73-6.61 |
| | 7-yl)pyrrolidin-2-yl)(4- | (m, 1H), 5.62 (brs, 1H), 4.96 (brs, 1H), 3.81-3.39 |
| | (2,2,3,3,3- | (m, 5H), 3.31-3.14 (m, 2H), 2.93-2.54 (m, J = |
| | pentafluoropropyl)piperazin-1- | 73.9 Hz, 4H), 2.31-2.17 (m, 1H), 2.16-2.06 (m, |
| | yl)methanone | 1H), 2.03-1.73 (m, 3H); LRMS (ES) m/z 515.3 |
| | | (M⁺ + 1). |
| 316 | (S)-2-((7-amino-2-(furan-2- | ¹H NMR (400 MHz, DMSO-d₆) δ 8.50-8.03 (m, |
| | yl)-[1,2,4]triazolo[1,5- | 2H), 7.92-7.83 (m, 1H), 7.56-7.39 (m, 1H), 7.11- |
| | a][1,3,5]triazin-5-yl)amino)-1- | 7.02 (m, 1H), 6.68 (dd, J = 3.3, 1.8 Hz, 1H), 6.36- |
| | (4-(2,2- | 6.00 (m, 1H), 4.95-4.76 (m, 1H), 3.68-3.39 (m, |
| | difluoroethyl)piperazin-1- | 5H), 2.86-2.65 (m, 3H), 2.57-2.43 (m, 2H), 1.27 |
| | yl)propan-1-one | (d, J = 6.8 Hz, 3H); LRMS (ES) m/z 422.4 (M⁺ + 1). |
| 317 | (S)-2-((7-amino-2-(furan-2- | ¹H NMR (400 MHz, DMSO-d₆) δ 8.48-8.03 (m, |
| | yl)-[1,2,4]triazolo[1,5- | 2H), 7.91-7.82 (m, 1H), 7.55-7.36 (m, 1H), 7.12- |
| | a][1,3,5]triazin-5-yl)amino)-1- | 7.02 (m, 1H), 6.68 (dd, J = 3.3, 1.8 Hz, 1H), 4.96- |
| | (4-(2,2- | 4.77 (m, 1H), 3.66-3.40 (m, 5H), 2.83-2.63 (m, |
| | difluoropropyl)piperazin-1- | 3H), 2.61-2.40 (m, 2H), 1.64 (td, J = 19.2, 4.2 Hz, |
| | yl)propan-1-one | 3H), 1.28 (d, J = 6.8 Hz, 3H); LRMS (ES) m/z |
| | | 436.5 (M⁺ + 1). |
| 318 | (S)-2-((7-amino-2-(furan-2- | ¹H NMR (400 MHz, DMSO-d₆) δ 8.50-8.02 (m, |
| | yl)-[1,2,4]triazolo[1,5- | 2H), 7.93-7.83 (m, 1H), 7.58-7.37 (m, 1H), 7.13- |
| | a][1,3,5]triazin-5-yl)amino)-1- | 7.01 (m, 1H), 6.68 (dd, J = 3.4, 1.8 Hz, 1H), 6.49 |
| | (4-(2,2,3,3- | (dt, J = 52.4, 5.7 Hz, 1H), 4.95-4.79 (m, 1H), 3.69- |
| | tetrafluoropropyl)piperazin-1- | 3.40 (m, 5H), 3.13-2.95 (m, 2H), 2.79-2.66 (m, |
| | yl)propan-1-one | 1H), 2.62-2.45 (m, 2H), 1.27 (d, J = 6.8 Hz, 3H); |
| | | LRMS (ES) m/z 472.5 (M⁺ + 1). |
| 319 | (S)-2-((7-amino-2-(furan-2- | ¹H NMR (400 MHz, DMSO-d₆) δ 8.48-8.04 (m, |
| | yl)-[1,2,4]triazolo[1,5- | 2H), 7.87 (dd, J = 1.7, 0.8 Hz, 1H), 7.59-7.38 (m, |
| | a][1,3,5]triazin-5-yl)amino)-1- | 1H), 7.12-7.02 (m, 1H), 6.68 (dd, J = 3.3, 1.8 Hz, |
| | (4-(2,2,3,3,3- | 1H), 4.94-4.77 (m, 1H), 3.68-3.38 (m, 4H), 3.32- |
| | pentafluoropropyl)piperazin-1- | 3.15 (m, 2H), 2.85-2.58 (m, 4H), 1.27 (d, J = 6.8 |
| | yl)propan-1-one | Hz, 3H); LRMS (ES) m/z 490.5 (M⁺ + 1). |
| 320 | (S)-2-((7-amino-2-(furan-2- | ¹H NMR (400 MHz, DMSO-d₆) δ 8.46-8.04 (m, |
| | yl)-[1,2,4]triazolo[1,5- | 2H), 7.87 (dd, J = 1.7, 0.8 Hz, 1H), 7.58-7.37 (m, |
| | a][1,3,5]triazin-5-yl)amino)-1- | 1H), 7.11-7.00 (m, 1H), 6.68 (dd, J = 3.3, 1.7 Hz, |
| | (4-(2,2,3,3,4,4,4- | 1H), 4.94-4.80 (m, 1H), 3.68-3.40 (m, 4H), 3.32- |
| | heptafluorobutyl)piperazin-1- | 3.19 (m, 2H), 2.84-2.58 (m, 4H), 1.28 (d, J = 6.8 |
| | yl)propan-1-one | Hz, 3H); LRMS (ES) m/z 540.5 (M⁺ + 1). |
| 362 | (S)-(1-(7-amino-2-(furan-2-yl)- | ¹H NMR (400 MHz, DMSO-d₆) δ 8.66-8.00 (m, |
| | [1,2,4]triazolo[1,5- | 2H), 7.90-7.83 (m, 1H), 7.09-6.99 (m, 1H), 6.70- |
| | a][1,3,5]triazin-5-yl)pyrrolidin- | 6.65 (m, 1H), 5.07-4.92 (m, 1H), 3.72-3.38 (m, |
| | 2-yl)(2-(2,2,2-trifluoroethyl)- | 5H), 3.30-3.08 (m, 7H), 2.34-2.18 (m, 1H), 2.10- |
| | 2,7-diazaspiro[3.5]nonan-7- | 1.85 (m, 3H), 1.85-1.75 (m, 1H), 1.74-1.63 (m, |
| | yl)methanone | 2H), 1.63-1.49 (m, 1H); LRMS (ES) m/z 506.1 |
| | | (M⁺ + 1). |
| 363 | (S)-(1-(7-amino-2-(furan-2-yl)- | ¹H NMR (400 MHz, DMSO-d₆) δ 8.63-8.07 (m, |
| | [1,2,4]triazolo[1,5- | 2H), 7.92-7.81 (m, 1H), 7.04 (dd, J = 19.3, 3.1 Hz, |
| | a][1,3,5]triazin-5-yl)pyrrolidin- | 1H), 6.72-6.62 (m, 1H), 4.58-4.41 (m, 1H), 4.40- |
| | 2-yl)(7-(2,2,2-trifluoroethyl)- | 4.09 (m, 1H), 3.91-3.75 (m, 1H), 3.70-3.47 (m, |
| | 2,7-diazaspiro[3-5]nonan-2- | 4H), 3.23-3.06 (m, 2H), 2.71-2.52 (m, 4H), 2.25- |
| | yl)methanone | 2.12 (m, 1H), 2.08-1.84 (m, 4H), 1.83-1.61 (m, |
| | | 4H); LRMS (ES) m/z 506.0 (M⁺ + 1). |
| 364 | (S)-(1-(7-amino-2-(furan-2-yl)- | ¹H NMR (400 MHz, DMSO-d₆) δ 8.61-8.01 (m, |
| | [1,2,4]triazolo[1,5- | 2H), 7.91-7.82 (m, 1H), 7.09-6.98 (m, 1H), 6.71- |
| | a][1,3,5]triazin-5-yl)pyrrolidin- | 6.62 (m, 1H), 5.05-4.93 (m, 1H), 3.72-3.38 (m, |
| | 2-yl)(2-(2,2-difluoropropyl)- | 5H), 3.32-3.26 (m, 1H), 3.23-3.02 (m, 4H), 2.82 |
| | 2,7-diazaspiro[3.5]nonan-7- | (t, J = 14.0 Hz, 2H), 2.33-2.16 (m, 1H), 2.09-1.76 |
| | yl)methanone | (m, 4H), 1.74-1.64 (m, 2H), 1.58 (t, J = 19.1 Hz, |
| | | 4H); LRMS (ES) m/z 506.0 (M⁺ + 1). |
| 365 | (S)-(1-(7-amino-2-(furan-2-yl)- | ¹H NMR (400 MHz, DMSO-d₆) δ 8.68-8.06 (m, |
| | [1,2,4]triazolo[1,5- | 2H), 7.93-7.82 (m, 1H), 7.12-6.96 (m, 1H), 6.74- |
| | a][1,3,5]triazin-5-yl)pyrrolidin- | 6.62 (m, 1H), 4.58-4.40 (m, 1H), 4.39-4.07 (m, |
| | 2-yl)(7-(2,2-difluoropropyl)- | 1H), 3.90-3.74 (m, 1H), 3.70-3.46 (m, 4H), 2.68 |
| | 2,7-diazaspiro[3-5]nonan-2- | (td, J = 14.0, 7.7 Hz, 2H), 2.48-2.35 (m, 3H), 2.26- |
| | yl)methanone | 2.12 (m, 1H), 2.08-1.84 (m, 4H), 1.82-1.52 (m, |
| | | 7H); LRMS (ES) m/z 502.1 (M⁺ + 1). |
| 366 | (S)-2-((7-amino-2-(furan-2- | ¹H NMR (400 MHz, DMSO-d₆) δ 8.54-7.98 (m, |
| | yl)-[1,2,4]triazolo[1,5- | 2H), 7.91-7.82 (m, 1H), 7.49 (d, J = 7.0 Hz, 1H), |
| | a][1,3,5]triazin-5-yl)amino)-1- | 7.12-6.99 (m, 1H), 6.68 (dd, J = 3.3, 1.8 Hz, 1H), |
| | (7-(2,2-difluoropropyl)-2,7- | 4.54-4.35 (m, 1H), 4.16 (dd, J = 36.2, 8.1 Hz, 1H), |
| | diazaspiro[3-5]nonan-2- | 3.84 (dd, J = 21.3, 8.2 Hz, 1H), 3.54 (q, J = 9.4 Hz, |
| | yl)propan-1-one | 2H), 2.67 (t, J = 14.0 Hz, 2H), 2.49-2.38 (m, 4H), |
| | | 1.86-1.51 (m, 7H), 1.27 (d, J = 7.0 Hz, 3H); LRMS |
| | | (ES) m/z 476.1 (M⁺ + 1). |
| 337 | (S)-(1-(7-amino-2-(furan-2-yl)- | ¹H NMR (400 MHz, DMSO-d₆) δ 8.29 (m, 2H), |
| | [1,2,4]triazolo[1,5- | 7.86 (d, J = 9.1 Hz, 1H), 7.13-6.93 (m, 1H), 6.67 |
| | a][1,3,5]triazin-5-yl)pyrrolidin- | (dd, J = 3.2, 1.7 Hz, 1H), 5.01 (d, J = 4.0 Hz, 1H), |
| | 2-yl)(4-(methyl(2,2,2- | 4.84-3.96 (m, 2H), 3.74-3.46 (m, 2H), 3.29- |
| | trifluoroethyl)amino)piperidin | 2.93 (m, 4H), 2.80-2.60 (m, 1H), 2.38 (m, 1H), |
| | -1-yl)methanone | 2.28 (m, 16.5 Hz, 1H), 2.01-1.83 (m, 4H), 1.83- |
| | | 1.48 (m, 4H), 1.32 (m, 1H); LRMS (ES) m/z 494.5 |
| | | (M⁺ + 1). |
| 345 | (S)-(1-(7-amino-2-(furan-2-yl)- | ¹H NMR (400 MHz, DMSO-d₆) δ 8.25 (s, 2H), 7.87 |
| | [1,2,4]triazolo[1,5- | (d, J = 1.7 Hz, 1H), 7.05 (dd, J = 11.9, 3.3 Hz, 1H), |
| | a][1,3,5]triazin-5-yl)pyrrolidin- | 6.68 (dd, J = 3.4, 1.8 Hz, 1H), 4.65 (dd, J = 66.0, |
| | 2-yl)(6-(2,2,2-trifluoroethyl)- | 9.0 Hz, 1H), 4.51-4.33 (m, 1H), 4.26 (dd, J = 27.7, |
| | 2,6-diazaspiro[3.3]heptan-2- | 8.8 Hz, 1H), 4.03-3.83 (m, 2H), 3.75-3.41 (m, |
| | yl)methanone | 6H), 3.24-3.04 (m, 2H), 2.24-2.07 (m, 1H), 2.05- |
| | | 1.78 (m, 3H); LRMS (ES) m/z 478.4 (M⁺ + 1). |
| 196 | ((S)-1-(7-amino-2-(5- | ¹H NMR (400 MHz, Chloroform-d) δ 7.08-6.96 |
| | methylfuran-2-yl)- | (m, 1H), 6.41 (s, 1H), 6.08 (dt, J = 16.0, 3.6 Hz, 1H), |
| | [1,2,4]triazolo[1,5- | 5.05-4.57 (m, 1H), 4.30-3.93 (m, 1H), 3.90- |
| | a][1,3,5]triazin-5-yl)pyrrolidin- | 3.62 (m, 2H), 3.60-3.27 (m, 1H), 3.09-2.51 (m, |
| | 2-yl)((R)-2-methyl-4-(2,2,2- | 5H), 2.42-2.22 (m, 4H), 2.19-1.81 (m, 3H), 1.53 |
| | trifluoroethyl)piperazin-1- | (dd, J = 67.9, 6.6 Hz, 1H), 1.20 (dd, J = 21.6, 6.8 Hz, |
| | yl)methanone | 1H). LRMS (ES) m/z 494.9 (M⁺ + 1). |
| 197 | (S)-(1-(7-amino-2-(5- | ¹H NMR (400 MHz, DMSO-d₆) δ 8.51-7.94(m, |
| | methylfuran-2-yl)- | 2H),6.99-6.88(m,1H),6.27(dt, J = 6.7, 3.0 Hz, 1H), |
| | [1,2,4]triazolo[1,5- | 4.98 (ddd, J = 16.2, 8.7, 3.0 Hz, 1H), 4.41-3.97 (m, |
| | a][1,3,5]triazin-5-yl)pyrrolidin- | 4H), 3.63 (ddp, J = 18.5, 12.5, 6.8 Hz, 2H), 3.09 (q, |
| | 2-yl)(1′-(2,2,2-trifluoroethyl)- | J = 10.4 Hz, 2H), 2.93 (t, J = 11.1 Hz, 2H), 2.36 (d, |
| | [4,4′-bipiperidin]-1- | J = 2.0 Hz, 3H), 2.25 (tt, J = 12.7, 5.5 Hz, 3H), 1.97- |
| | yl)methanone | 1.55 (m, 8H), 1.41-0.87 (m, 6H). LRMS (ES) m/z |
| | | 562.1 (M⁺ + 1). |
| 204 | ((S)-1-(7-amino-2-(furan-2-yl)- | ¹H NMR (400 MHz, DMSO-d₆) δ 8.32 (d, J = 107.6 |
| | [1,2,4]triazolo[1,5- | Hz, 2H), 7.87 (d, J = 2.1 Hz, 1H), 7.12-6.98 (m, |
| | a][1,3,5]triazin-5-yl)pyrrolidin- | 1H), 6.68 (tt, J = 3.3, 1.5 Hz, 1H), 4.93 (ddd, J = |
| | 2-yl)(8-(2,2,2-trifluoroethyl)- | 90.7, 8.5, 3.3 Hz, 1H), 4.02-3.53 (m, 4H), 3.35- |
| | 3,8-diazabicyclo[3.2.1]octan-3- | 3.06 (m, 5H), 2.89-2.74 (m, 1H), 2.39-2.12 (m, |
| | yl)methanone | 1H), 2.05-1.72 (m, 5H), 1.63-1.33 (m, 2H). LRMS |
| | | (ES) m/z 492.3 (M⁺ + 1). |
| 205 | ((S)-1-(7-amino-2-(furan-2-yl)- | ¹H NMR (400 MHz, DMSO-d₆) δ 8.55-8.01 (m, |
| | [1,2,4]triazolo[1,5- | 2H),7.87(ddd, J = 5.7, 1.8, 0.8 Hz, 1H), 7.11-6.99 |
| | a][1,3,5]triazin-5-yl)pyrrolidin- | (m, 1H), 6.67 (ddt, J = 5.1, 3.5, 1.8 Hz, 1H), 5.10- |
| | 2-yl)((R)-2-methyl-4-(2,2,2- | 4.79 (m, 1H), 4.55-3.78 (m, 2H), 3.70-3.55 (m, |
| | trifluoroethyl)piperazin-1- | 2H), 3.35-3.10 (m, 3H), 2.98-2.61 (m, 3H), 2.41- |
| | yl)methanone | 2.13 (m, 2H), 2.00-1.69 (m, 3H), 1.50 (dd, J = |
| | | 15.6, 6.6 Hz, 2H), 1.14 (d, J = 6.8 Hz, 1H). LRMS |
| | | (ES) m/z 492.4 (M⁺ + 1). |
| 206 | (S)-(1-(7-amino-2-(furan-2-yl)- | ¹H NMR (400 MHz, DMSO-d₆) δ 8.56-7.99 |
| | [1,2,4]triazolo[1,5- | (m,2H), 7.87 (q, J = 2.3 Hz, 1H), 7.10-6.96 (m, |
| | a][1,3,5]triazin-5-yl)pyrrolidin- | 1H), 6.71-6.61 (m, 1H), 5.06-4.93 (m, 1H), 4.35 |
| | 2-yl)(1′-(2,2,2-trifluoroethyl)- | (t, J = 14.3 Hz, 1H), 4.19-3.96 (m, 1H), 3.62 (dq, J = |
| | [4,4′-bipiperidin]-1- | 18.3, 6.8 Hz, 2H), 3.19-3.05 (m, 3H), 2.93 (d, J = |
| | yl)methanone | 11.4 Hz, 2H), 2.26 (q, J = 13.4, 11.2 Hz, 3H), 1.99- |
| | | 1.56 (m, 8H), 1.42-0.90 (m, 6H). |
| 225 | (S)-(1-(7-amino-2-(furan-2-yl)- | ¹H NMR (400 MHz, DMSO-d₆) δ 8.33 (d, J = 125.5 |
| | [1,2,4]triazolo[1,5- | Hz, 2H), 7.88 (td, J = 1.9, 0.8 Hz, 1H), 7.06 (ddd, J = |
| | a][1,3,5]triazin-5-yl)pyrrolidin- | 5.0, 3.3, 0.8 Hz, 1H), 6.68 (dt, J = 3.3, 1.6 Hz, 1H), |
| | 2-yl)(4-(2,2- | 4.98 (ddd, J = 12.5, 8.6, 3.1 Hz, 1H), 4.12 (q, J = 5.3 |
| | difluorobutyl)piperazin-1- | Hz, 1H), 3.70-3.51 (m, 5H), 3.18 (d, J = 5.2 Hz, |
| | yl)methanone | 2H), 2.80 (td, J = 14.2, 7.9 Hz, 3H), 2.55 (s, 2H), |
| | | 2.48-2.37 (m, 1H), 2.30-2.18 (m, 1H), 2.05- |
| | | 1.78 (m, 5H), 0.97 (td, J = 7.5, 0.9 Hz, 3H). LRMS |
| | | (ES) m/z 476.3 (M⁺ + 1). |
| 226 | (S)-(1-(7-amino-2-(furan-2-yl)- | ¹H NMR (400 MHz, DMSO-d₆) δ 8.33 (d, J = 125.9 |
| | [1,2,4]triazolo[1,5- | Hz, 2H), 7.88 (s, 1H), 7.06 (s, 1H), 6.63 (d, J = 41.3 |
| | a][1,3,5]triazin-5-yl)pyrrolidin- | Hz, 2H), 5.00 (d, J = 10.2 Hz, 1H), 3.77-3.47 (m, |
| | 2-yl)(4-(2,2,3,3- | 5H), 3.24-2.74 (m, 4H), 2.36-1.71 (m, 5H), 1.49 - |
| | tetrafluoropropyl)piperazin-1- | 0.69 (m, 1H). LRMS (ES) m/z 498.3 (M⁺ + 1). |
| | yl)methanone | |
| 227 | (S)-(1-(7-amino-2-(furan-2-yl)- | ¹H NMR (400 MHz, DMSO-d₆) δ 8.33 (d, J = 127.6 |
| | [1,2,4]triazolo[1,5- | Hz, 2H), 7.88 (ddd, J = 3.6, 1.8, 0.8 Hz, 1H), 7.05 |
| | a][1,3,5]triazin-5-yl)pyrrolidin- | (ddd, J = 9.8, 3.4, 0.8 Hz, 1H), 6.68 (dt, J = 3.4, 1.7 |
| | 2-yl)(4-(2,2,3,3,3- | Hz, 1H), 4.98 (ddd, J = 14.7, 8.6, 3.2 Hz, 1H), 3.72 - |
| | pentafluoropropyl)piperazin-1- | 3.39 (m, 6H), 3.33-3.22 (m, 2H), 2.99-2.82 |
| | yl)methanone | (m, 1H), 2.71-2.54 (m, 4H), 2.26 (ddd, J = 12.1, |
| | | 8.2, 3.7 Hz, 1H), 1.97-1.77 (m, 3H). LRMS (ES) |
| | | m/z 516.3 (M⁺ + 1). |
| 228 | (S)-(1-(7-amino-2-(furan-2-yl)- | ¹H NMR (400 MHz, DMSO-d₆) δ 8.17 (s,2H), 7.87 |
| | [1,2,4]triazolo[1,5- | (ddd, J = 4.1, 1.8, 0.8 Hz, 1H), 7.06 (ddd, J = 9.9, |
| | a][1,3,5]triazin-5-yl)pyrrolidin- | 3.4, 0.8 Hz, 1H), 6.67 (dt, J = 3.5, 1.9 Hz, 1H), 4.97 |
| | 2-yl)(4-(2,2,3,3,4,4,4- | (ddd, J = 13.8, 8.6, 3.2 Hz, 1H), 3.72-3.41 (m, 6H), |
| | heptafluorobutyl)piperazin-1- | 3.36-3.24 (m, 2H), 3.00-2.80 (m, 1H), 2.55 (s, |
| | yl)methanone | 4H), 2.31-2.20 (m, 1H), 1.97-1.77 (m, 3H). LRMS |
| | | (ES) m/z 566.2 (M⁺ + 1). |
| 252 | (S)-(1-(7-amino-2-(furan-2-yl)- | ¹H NMR (400 MHz, DMSO-d₆) δ 8.35 (d, J = 59.8 |
| | [1,2,4]triazolo[1,5- | Hz, 3H), 7.87 (d, J = 1.9 Hz, 1H), 7.75-7.63 (m, |
| | a][1,3,5]triazin-5-yl)azetidin-2- | 1H), 7.04 (dd, J = 7.9, 3.5 Hz, 1H), 6.68 (dd, J = 3.4, |
| | yl)(4-(2,2- | 1.8 Hz, 1H), 5.24 (s, 1H), 4.08-3.92 (m, 2H), 3.50 |
| | difluoroethyl)piperazin-1- | (d, J = 53.4 Hz, 5H), 2.70-2.58 (m, 3H), 1.63 (d, J= |
| | yl)methanone | 18.8 Hz, 1H), 0.89-0.77 (m, 3H). LRMS (ES) |
| | | m/z 433.42 (M⁺ + 1). |
| 253 | (S)-(1-(7-amino-2-(furan-2-yl)- | ¹H NMR (400 MHz, DMSO-d₆) δ 8.64-8.15 (m, |
| | [1,2,4]triazolo[1,5- | 2H), 7.99 (s, 1H), 7.87 (d, J = 1.8 Hz, 1H), 7.04 (d, |
| | a][1,3,5]triazin-5-yl)azetidin-2- | J = 4.6 Hz, 1H), 6.67 (dd, J = 3.4, 1.8 Hz, 1H), 5.21 |
| | yl)(4-(2,2- | (dd, J = 9.2, 5.2 Hz, 1H), 4.11-3.95 (m, 2H), 3.59- |
| | difluoropropyl)piperazin-1- | 3.39 (m, 4H), 3.17 (d, J = 3.1 Hz, 2H), 2.70 (dt, J = |
| | yl)methanone | 56.1, 13.9 Hz, 5H), 1.65 (t, J = 19.2 Hz, 3H), 1.43- |
| | | 1.22 (m, 2H). LRMS (ES) m/z 448.4 (M⁺ + 1). |
| 254 | (S)-(1-(7-amino-2-(furan-2-yl)- | ¹H NMR (400 MHz, DMSO-d₆) δ 8.34 (d, J = 80.2 |
| | [1,2,4]triazolo[1,5- | Hz, 2H), 7.87 (d, J = 1.8 Hz, 1H), 7.05 (q, J = 3.6 |
| | a][1,3,5]triazin-5-yl)azetidin-2- | Hz, 1H), 6.67 (dd, J = 3.4, 1.8 Hz, 1H), 5.21 (dd, J = |
| | yl)(4-(2,2- | 9.2, 5.3 Hz, 1H), 4.18-3.92 (m, 3H), 3.53 (d, J = |
| | difluorobutyl)piperazin-1- | 17.1 Hz, 1H), 3.17 (d, J = 2.6 Hz, 3H), 2.77 (t, J = |
| | yl)methanone | 14.1 Hz, 2H), 2.64 (d, J = 16.1 Hz, 3H), 2.54 (s, 1H), |
| | | 2.11 (s, 1H), 1.96 (dq, J = 17.5, 8.5 Hz, 2H), 0.96 (t, |
| | | J = 7.5 Hz, 3H). LRMS (ES) m/z 462.3 (M⁺ + 1). |
| 255 | (S)-(1-(7-amino-2-(furan-2-yl)- | ¹H NMR (400 MHz, DMSO-d₆) δ 8.41 (d, J = 127.2 |
| | [1,2,4]triazolo[1,5- | Hz, 2H), 7.87 (ddd, J = 3.9, 1.8, 0.8 Hz, 1H), 7.04 |
| | a][1,3,5]triazin-5-yl)azetidin-2- | (dd, J = 6.8, 3.0 Hz, 1H), 6.72-6.39 (m, 2H), 5.21 |
| | yl)(4-(2,2,3,3- | (dd, J = 9.1, 5.3 Hz, 1H), 4.00 (s, 2H), 3.72-3.35 |
| | tetrafluoropropyl)piperazin-1- | (m, 4H), 3.29-3.13 (m, 1H), 3.03 (dd, J = 28.1, 12.7 |
| | yl)methanone | Hz, 3H), 2.77-2.56 (m, 3H), 2.11 (s, 1H). LRMS |
| | | (ES) m/z 484.4 (M⁺ + 1). |
| 256 | (S)-(1-(7-amino-2-(furan-2-yl)- | ¹H NMR (400 MHz, DMSO-d₆) δ 8.66-8.12 (m, |
| | [1,2,4]triazolo[1,5- | 2H), 7.87 (d, J = 1.7 Hz, 1H), 7.10-6.99 (m, 1H), |
| | a][1,3,5]triazin-5-yl)azetidin-2- | 6.67 (dd, J = 3.3, 1.8 Hz, 1H), 5.21 (dd, J = 9.2, 5.3 |
| | yl)(4-(2,2,3,3,3- | Hz, 1H), 4.12-3.90 (m, 2H), 3.52 (dd, J = 57.6, |
| | pentafluoropropyl)piperazin-1- | 40.7 Hz, 4H), 3.25 (d, J = 16.2 Hz, 2H), 3.17 (d, J = |
| | yl)methanone | 5.0 Hz, 1H), 2.83-2.57 (m, 4H), 2.11 (s, 1H). LRMS |
| | | (ES) m/z 502.3 (M⁺ + 1). |
| 257 | (S)-(1-(7-amino-2-(furan-2-yl)- | ¹H NMR (400 MHz, DMSO-d₆) δ 8.42 (d, J = 121.5 |
| | [1,2,4]triazolo[1,5- | Hz, 2H), 7.87 (d, J = 1.7 Hz, 1H), 7.09-6.98 (m, |
| | a][1,3,5]triazin-5-yl)azetidin-2- | 1H), 6.67 (dt, J = 5.5, 2.7 Hz, 1H), 5.22 (dd, J = 9.1, |
| | yl)(4-(2,2,3,3,4,4,4- | 5.3 Hz, 1H), 3.98 (dt, J = 16.6, 7.2 Hz, 2H), 3.56 (d, |
| | heptafluorobutyl)piperazin-1- | J = 59.8 Hz, 4H), 3.27 (s, 1H), 2.70 (d, J = 68.0 Hz, |
| | yl)methanone | 6H), 2.12 (s, 1H). LRMS (ES) m/z 522.3 (M⁺ + 1). |
| 278 | (S)-(1-(7-amino-2-(furan-2-yl)- | ¹H NMR (400 MHz, DMSO-d₆) δ 8.61-8.01 (m, |
| | [1,2,4]triazolo[1,5- | 2H), 7.89-7.81 (m, 1H), 7.12-6.97 (m, 1H), 6.68 |
| | a][1,3,5]triazin-5-yl)pyrrolidin- | (ddd, J = 4.8, 3.3, 1.6 Hz, 1H), 4.95 (ddt, J = 8.9, |
| | 2-yl)(4-(2,2,2-trifluoroethyl)- | 7.1, 3.1 Hz, 1H), 3.90-3.37 (m, 7H), 3.30-3.09 |
| | 1,4-diazepan-1-yl)methanone | (m, 2H), 3.06-2.81 (m, 3H), 2.30 (ddt, J = 19.5, |
| | | 12.6, 6.8 Hz, 1H), 2.05-1.63 (m, 5H). LRMS (ES) |
| | | m/z 480.6 (M⁺ + 1). |
| 279 | (S)-(1-(7-amino-2-(furan-2-yl)- | ¹H NMR (400 MHz, DMSO-d₆) δ 8.38 (d, J = 57.0 |
| | [1,2,4]triazolo[1,5- | Hz, 2H), 7.88 (d, J = 1.8 Hz, 1H), 7.07 (d, J = 3.3 |
| | a][1,3,5]triazin-5-yl)piperidin- | Hz, 1H), 6.74-6.37 (m, 2H), 5.57 (t, J = 6.0 Hz, |
| | 2-y1)(4-(2,2,3,3- | 1H), 4.65-4.44 (m, 1H), 3.67-3.36 (m, 5H), 3.04 |
| | tetrafluoropropyl)piperazin-1- | (t, J = 15.0 Hz, 2H), 2.70 (d, J = 17.1 Hz, 2H), 1.94- |
| | yl)methanone | 1.34 (m, 7H). LRMS (ES) m/z 512.5 (M⁺ + 1). |
| 280 | (S)-(1-(7-amino-2-(furan-2-yl)- | ¹H NMR (400 MHz, DMSO-d₆) δ 8.38 (d, J = 61.8 |
| | [1,2,4]triazolo[1,5- | Hz, 2H), 7.88 (d, J = 1.7 Hz, 1H), 7.07 (d, J = 3.8 |
| | a][1,3,5]triazin-5-yl)piperidin- | Hz, 1H), 6.68 (dd, J = 3.4, 1.8 Hz, 1H), 5.57 (d, J = |
| | 2-yl)(4-(2,2,3,3,3- | 8.6 Hz, 1H), 4.54 (t, J = 16.9 Hz, 1H), 3.53 (d, J = |
| | pentafluoropropyl)piperazin-1- | 14.2 Hz, 4H), 3.32-3.15 (m, 3H), 2.63 (d, J = 39.5 |
| | yl)methanone | Hz, 4H), 1.94-1.17 (m, 7H). LRMS (ES) m/z 530.4 |
| | | (M⁺ + 1). |
| 287 | (S)-(1-(7-amino-2-(furan-2-yl)- | ¹H NMR (400 MHz, DMSO-d₆) δ 8.59-8.04 (m, |
| | [1,2,4]triazolo[1,5- | 2H), 7.87 (d, J = 4.3 Hz, 1H), 7.09-6.98 (m, 1H), |
| | a][1,3,5]triazin-5-yl)pyrrolidin- | 6.67 (dd, J = 3.4, 1.8 Hz, 1H), 6.13 (t, J = 55.8 Hz, |
| | 2-yl)(1′-(2,2-difluoroethyl)- | 1H), 5.01 (ddd, J = 15.0, 8.6, 3.2 Hz, 1H), 4.35 (t, J = |
| | [4,4′-bipiperidin]-1- | 14.9 Hz, 1H), 4.09 (q, J = 16.8, 16.2 Hz, 1H), 3.74- |
| | yl)methanone | 3.54 (m, 2H), 3.21-2.88 (m, 3H), 2.78-2.58 |
| | | (m, 2H), 2.36-2.01 (m, 3H), 1.97-1.59 (m, 7H), |
| | | 1.37-0.96 (m, 7H). LRMS (ES) m/z 530.6 (M⁺ + 1). |
| 288 | (S)-(1-(7-amino-2-(furan-2-yl)- | ¹H NMR (400 MHz, DMSO-d₆) δ 8.27 (d, J = 133.3 |
| | [1,2,4]triazolo[1,5- | Hz, 2H), 7.87 (t, J = 3.9 Hz, 1H), 7.11-6.94 (m, |
| | a][1,3,5]triazin-5-yl)pyrrolidin- | 1H), 6.71-6.61 (m, 1H), 5.07-4.91 (m, 1H), 4.44- |
| | 2-yl)(1′-(2,2-difluoropropyl)- | 3.96 (m, 2H), 3.62 (dq, J = 18.7, 7.0 Hz, 2H), 3.19- |
| | [4,4′-bipiperidin]-1- | 2.86 (m, 3H), 2.67 (q, J = 13.8, 13.4 Hz, 2H), 2.35- |
| | yl)methanone | 2.05 (m, 3H), 1.99-1.54 (m, 10H), 1.42-0.89 |
| | | (m, 6H). LRMS (ES) m/z 544.6 (M⁺ + 1). |
| 289 | (S)-(1-(7-amino-2-(furan-2-yl)- | ¹H NMR (400 MHz, DMSO-d₆) δ 8.56-8.00 (m, |
| | [1,2,4]triazolo[1,5- | 2H), 7.91-7.83 (m, 1H), 7.10-6.96 (m, 1H), 6.70- |
| | a][1,3,5]triazin-5-yl)pyrrolidin- | 6.31 (m, 2H), 5.01 (t, J = 10.4 Hz, 1H), 4.36 (t, J = |
| | 2-yl)(1′-(2,2,3,3- | 14.1 Hz, 1H), 4.11 (t, J = 15.4 Hz, 1H), 3.74-3.53 |
| | tetrafluoropropyl)-[4,4′- | (m, 2H), 3.19-2.84 (m, 5H), 2.23 (dt, J = 23.9, 12.2 |
| | bipiperidin]-1-yl)methanone | Hz, 3H), 1.97-1.54 (m, 8H), 1.41-0.90 (m, 6H). |
| | | LRMS (ES) m/z 580.6 (M⁺ + 1). |
| 297 | (S)-(1-(7-amino-2-(5- | ¹H NMR (400 MHz, DMSO-d₆) δ 8.28 (d, J = 117.1 |
| | methylfuran-2-yl)- | Hz, 2H), 6.94 (dd, J = 7.8, 3.3 Hz, 1H), 6.40-6.02 |
| | [1,2,4]triazolo[1,5- | (m, 2H), 4.99 (ddd, J = 11.7, 8.6, 3.2 Hz, 1H), 3.74- |
| | a][1,3,5]triazin-5-yl)pyrrolidin- | 3.47 (m, 5H), 2.83 (tdd, J = 15.6, 14.1, 4.4 Hz, |
| | 2-yl)(4-(2,2- | 3H), 2.59 (s, 1H), 2.47-2.32 (m, 4H), 2.26 (ddt, J = |
| | difluoroethyl)piperazin-1- | 12.2, 8.1, 4.0 Hz, 1H), 1.97-1.76 (m, 3H). LRMS |
| | yl)methanone | (ES) m/z 462.5 (M⁺ + 1). |
| 311 | (S)-(1-(7-amino-2-(5- | ¹H NMR 1H NMR (400 MHz, DMSO-d₆) δ 8.15 (s, |
| | methylfuran-2-yl)- | 2H), 6.94 (dd, J = 4.8, 3.3 Hz, 1H), 6.29 (dd, J = |
| | [1,2,4]triazolo[1,5- | 3.3, 1.5 Hz, 1H), 4.97 (ddd, J = 11.5, 8.6, 3.1 Hz, 1H), |
| | a][1,3,5]triazin-5-yl)pyrrolidin- | 4.12 (q, J = 5.2 Hz, 1H), 3.72-3.49 (m, 5H), 3.17 |
| | 2-yl)(4-(2,2- | (d, J = 5.0 Hz, 2H), 2.80 (td, J = 14.0, 8.1 Hz, 3H), |
| | difluoropropyl)piperazin-1- | 2.59 (d, J = 6.1 Hz, 1H), 2.48 (s, 1H), 2.36 (s, 3H), |
| | yl)methanone | 2.24 (td, J = 5.6, 2.9 Hz, 1H), 1.97-1.77 (m, 3H), |
| | | 1.66 (td, J = 19.1, 1.8 Hz, 3H). LRMS (ES) m/z 476.5 |
| | | (M⁺ + 1). |
| 298 | (S)-(1-(7-amino-2-(5- | ¹H NMR (400 MHz, DMSO-d₆) δ 8.13 (s, 2H), 6.94 |
| | methylfuran-2-yl)- | (dd, J = 7.0, 3.2 Hz, 1H), 6.74-6.39 (m, 1H), 6.30 |
| | [1,2,4]triazolo[1,5- | (ddd, J = 3.2, 1.9, 1.1 Hz, 1H), 4.98 (td, J = 10.2, 9.4, |
| | a][1,3,5]triazin-5-yl)pyrrolidin- | 3.2 Hz, 1H), 4.10 (q, J = 5.3 Hz, 1H), 3.70-3.50 (m, |
| | 2-yl)(4-(2,2,3,3- | 5H), 3.20-3.01 (m, 4H), 2.60 (s, 2H), 2.39-2.21 |
| | tetrafluoropropyl)piperazin-1- | (m, 4H), 1.96-1.79 (m, 3H). LRMS (ES) m/z 512.5 |
| | yl) methanone | (M⁺ + 1). |
| 299 | (S)-(1-(7-amino-2-(5- | ¹H NMR (400 MHz, DMSO-d₆) δ 8.13 (s, 2H), 6.93 |
| | methylfuran-2-yl)- | (dd, J = 9.6, 3.2 Hz, 1H), 6.35-6.26 (m, 1H), 4.98 |
| | [1,2,4]triazolo[1,5- | (ddd, J = 12.7, 8.5, 3.2 Hz, 1H), 4.26-4.04 (m, 1H), |
| | a][1,3,5]triazin-5-yl)pyrrolidin- | 3.73-3.38 (m, 6H), 3.18 (d, J = 5.3 Hz, 2H), 2.89 |
| | 2-yl)(4-(2,2,3,3,3- | (d, J = 42.0 Hz, 1H), 2.65 (s, 3H), 2.36 (d, J = 3.1 |
| | pentafluoropropyl)piperazin-1- | Hz, 3H), 2.25 (dtd, J = 12.6, 8.3, 4.1 Hz, 1H), 1.98- |
| | yl)methanone | 1.76 (m, 3H), 0.84 (td, J = 8.1, 7.3, 3.1 Hz, 1H). |
| | | LRMS (ES) m/z 530.5 (M⁺ + 1). |
| 314 | (S)-(1-(7-amino-2-(5- | ¹H NMR (400 MHz, DMSO-d₆) δ 8.69-8.03 (m, |
| | methylfuran-2-yl)- | 2H), 6.93 (t, J = 5.3 Hz, 1H), 6.29 (dd, J = 3.2, 1.3 |
| | [1,2,4]triazolo[1,5- | Hz, 1H), 5.21 (dd, J = 9.2, 5.3 Hz, 1H), 4.00 (s, 2H), |
| | a][1,3,5]triazin-5-yl)azetidin-2- | 3.71-3.37 (m, 4H), 2.78 (t, J = 13.9 Hz, 2H), 2.71- |
| | yl)(4-(2,2- | 2.54 (m, 3H), 2.36 (s, 3H), 2.10 (d, J = 6.5 Hz, |
| | difluoropropyl)piperazin-1- | 1H), 1.65 (t, J = 19.1 Hz, 3H), 1.24 (s, 1H), 0.89- |
| | yl)methanone | 0.79 (m, 1H). LRMS (ES) m/z 462.6 (M⁺ + 1). |
| 312 | (S)-(1-(7-amino-2-(5- | ¹H NMR (400 MHz, DMSO-d₆) δ 8.41 (d, J = 107.1 |
| | methylfuran-2-yl)- | Hz, 2H), 6.92 (s, 1H), 6.56 (t, J = 52.5 Hz, 1H), 6.30 |
| | [1,2,4]triazolo[1,5- | (dd, J = 3.3, 1.2 Hz, 1H), 5.22 (dd, J = 9.2, 5.3 Hz, |
| | a][1,3,5]triazin-5-yl)azetidin-2- | 1H), 4.10 (q, J = 5.2 Hz, 1H), 4.00 (s, 2H), 3.47 (d, |
| | y1)(4-(2,2,3,3- | J = 38.8 Hz, 4H), 3.06 (t, J = 15.2 Hz, 2H), 2.65 (d, |
| | tetrafluoropropyl)piperazin-1- | J = 21.8 Hz, 3H), 2.36 (s, 3H), 2.10 (d, J = 7.7 Hz, |
| | yl)methanone | 1H). LRMS (ES) m/z 498.6 (M⁺ + 1). |
| 313 | (S)-(1-(7-amino-2-(5- | ¹H NMR (400 MHz, DMSO-d₆) δ 8.26 (s, 2H), 6.93 |
| | methylfuran-2-yl)- | (d, J = 8.5 Hz, 1H), 6.30 (dd, J = 3.3, 1.1 Hz, 1H), |
| | [1,2,4]triazolo[1,5- | 5.22 (dd, J = 9.2, 5.3 Hz, 1H), 4.10 (q, J = 5.2 Hz, |
| | a][1,3,5]triazin-5-yl)azetidin-2- | 1H), 4.00 (s, 2H), 3.70-3.39 (m, 4H), 3.26 (d, J = |
| | yl)(4-(2,2,3,3,3- | 16.3 Hz, 2H), 2.70 (d, J = 66.5 Hz, 5H), 2.36 (s, 3H), |
| | pentafluoropropyl)piperazin-1- | 2.11 (d, J = 10.3 Hz, 1H). LRMS (ES) m/z 516.6 |
| | yl)methanone | (M⁺ + 1). |
| 323 | (S)-(1-(7-amino-2-(furan-2-yl)- | ¹H NMR (400 MHz, DMSO-d₆) δ 8.71-8.05 (m, |
| | [1,2,4]triazolo[1,5- | 2H), 7.87 (dd, J = 1.8, 0.8 Hz, 1H), 7.04 (d, J = 10.3 |
| | a][1,3,5]triazin-5-yl)azetidin-2- | Hz, 1H), 6.68 (dd, J = 3.4, 1.8 Hz, 1H), 6.11 (ddt, J = |
| | yl)(1′-(2,2-difluoroethyl)-[4,4′- | 60.2, 55.8, 4.4 Hz, 1H), 5.21 (d, J = 8.3 Hz, 1H), |
| | bipiperidin]-1-yl)methanone | 4.40 (d, J = 12.7 Hz, 1H), 4.09-3.63 (m, 3H), 3.09- |
| | | 2.82 (m, 3H), 2.68 (ddt, J = 15.9, 11.2, 5.7 Hz, |
| | | 3H), 2.14-1.98 (m, 3H), 1.65 (dd, J = 34.2, 12.7 Hz, |
| | | 4H), 1.13 (ddd, J = 72.1, 36.2, 24.5 Hz, 7H). LRMS |
| | | (ES) m/z 516.7 (M⁺ + 1). |
| 324 | (S)-(1-(7-amino-2-(furan-2-yl)- | ¹H NMR (400 MHz, DMSO-d₆) δ 8.41 (d, J = 121.2 |
| | [1,2,4]triazolo[1,5- | Hz, 2H), 7.87 (dd, J = 1.8, 0.9 Hz, 1H), 7.04 (d, J = |
| | a][1,3,5]triazin-5-yl)azetidin-2- | 11.1 Hz, 1H), 6.68 (dd, J = 3.4, 1.8 Hz, 1H), 5.21 (d, |
| | yl)(1′-(2,2-difluoropropyl)- | J = 8.7 Hz, 1H), 4.40 (d, J = 12.9 Hz, 1H), 4.08- |
| | [4,4′-bipiperidin]-1- | 3.68 (m, 3H), 3.09-2.83 (m, 3H), 2.66 (td, J = |
| | yl)methanone | 14.0, 5.9 Hz, 3H), 2.10 (t, J = 11.4 Hz, 3H), 1.79- |
| | | 1.52 (m, 7H), 1.46-0.91 (m, 7H). LRMS (ES) m/z |
| | | 530.6 (M⁺ + 1). |
| 325 | (S)-(1-(7-amino-2-(furan-2-yl)- | ¹H NMR (400 MHz, DMSO-d₆) δ 8.25 (s, 2H), 7.87 |
| | [1,2,4]triazolo[1,5- | (dd, J = 1.9, 0.8 Hz, 1H), 7.10-6.95 (m, 1H), 6.68 |
| | a][1,3,5]triazin-5-yl)azetidin-2- | (dd, J = 3.4, 1.8 Hz, 1H), 6.46 (dd, J = 55.9, 48.3 |
| | yl)(1′-(2,2,3,3- | Hz, 1H), 5.21 (d, J = 8.8 Hz, 1H), 4.40 (d, J = 12.8 |
| | tetrafluoropropyl)-[4,4′- | Hz, 1H), 4.10-3.65 (m, 3H), 3.10-2.82 (m, 5H), |
| | bipiperidin]-1-yl)methanone | 2.72-2.59 (m, 1H), 2.30-2.03 (m, 3H), 1.65 (dd, |
| | | J = 35.6, 12.8 Hz, 4H), 1.47-0.92 (m, 6H). LRMS |
| | | (ES) m/z 566.6 (M⁺ + 1). |
| 326 | (S)-(1-(7-amino-2-(furan-2-yl)- | ¹H NMR (400 MHz, DMSO-d₆) δ 8.67-8.04 (m, |
| | [1,2,4]triazolo[1,5- | 2H), 7.87 (dd, J = 1.8, 0.9 Hz, 1H), 7.08-6.94 (m, |
| | a][1,3,5]triazin-5-yl)azetidin-2- | 1H), 6.68 (dd, J = 3.6, 1.9 Hz, 1H), 5.21 (d, J = 8.7 |
| | yl)(1′-(2,2,3,3,3- | Hz, 1H), 4.40 (d, J = 12.9 Hz, 1H), 4.09-3.62 (m, |
| | pentafluoropropyl)-[4,4′- | 3H), 3.21-2.83 (m, 5H), 2.72-2.58 (m, 1H), 2.26 |
| | bipiperidin]-1-yl)methanone | (q, J = 11.4 Hz, 2H), 2.09 (s, 1H), 1.66 (dd, J = 30.5, |
| | | 12.8 Hz, 4H), 1.50-0.92 (m, 7H). LRMS (ES) m/z |
| | | 584.7 (M⁺ + 1). |
| 334 | (S)-2-((7-amino-2-(furan-2- | ¹H NMR (400 MHz, DMSO-d₆) δ 8.25 (s, 2H), 7.87 |
| | yl)-[1,2,4]triazolo[1,5- | (dd, J = 1.8, 0.8 Hz, 1H), 7.44-7.29 (m, 1H), 7.05 |
| | a][1,3,5]triazin-5-yl)amino)-1- | (dd, J = 9.7, 3.8 Hz, 1H), 6.68 (dd, J = 3.4, 1.8 Hz, |
| | (1′-(2,2-difluoroethyl)-[4,4′- | 1H), 6.10 (tt, J = 56.0, 4.3 Hz, 1H), 4.88 (d, J = 10.5 |
| | bipiperidin]-1-yl)propan-1-one | Hz, 1H), 4.40 (d, J = 12.8 Hz, 1H), 4.01 (d, J = 13.6 |
| | | Hz, 1H), 2.96 (dd, J = 48.4, 11.7 Hz, 3H), 2.66 (td, |
| | | J = 15.7, 4.3 Hz, 2H), 2.05 (t, J = 11.3 Hz, 2H), 1.80- |
| | | 1.55 (m, 4H), 1.39-0.88 (m, 9H). LRMS (ES) |
| | | m/z 504.6 (M⁺ + 1). |
| 335 | (S)-2-((7-amino-2-(furan-2- | ¹H NMR (400 MHz, DMSO-d₆) δ 8.26 (s, 2H), 7.87 |
| | yl)-[1,2,4]triazolo[1,5- | (d, J = 1.8 Hz, 1H), 7.37 (dd, J = 21.1, 7.5 Hz, 1H), |
| | a][1,3,5]triazin-5-yl)amino)-1- | 7.05 (dd, J = 13.0, 3.8 Hz, 1H), 6.67 (dd, J = 3.4, 1.8 |
| | (1′-(2,2-difluoropropyl)-[4,4′- | Hz, 1H), 4.88 (t, J = 7.2 Hz, 1H), 4.40 (d, J = 12.7 |
| | bipiperidin]-1-yl)propan-1-one | Hz, 1H), 4.01 (d, J = 13.6 Hz, 1H), 2.95 (dd, J = 51.6, |
| | | 11.6 Hz, 3H), 2.67 (s, 2H), 2.17-2.02 (m, 2H), 1.79- |
| | | 1.38 (m, 8H), 1.37-0.91 (m, 10H). LRMS (ES) |
| | | m/z 518.6 (M⁺ + 1). |
| 341 | (S)-(1-(7-amino-2-(furan-2-yl)- | ¹H NMR (400 MHz, DMSO-d₆) δ 8.38 (d, J = 102.8 |
| | [1,2,4]triazolo[1,5- | Hz, 1H), 7.86 (d, J = 1.8 Hz, 1H), 7.10-6.90 (m, |
| | a][1,3,5]triazin-5-yl)pyrrolidin- | 1H), 6.77-6.64 (m, 1H), 6.52-6.06 (m, 1H), 5.11 |
| | 2-yl)(7-(2,2-difluoroethyl)-4,7- | (s, 1H), 4.19 (s, 1H), 3.66 (d, J = 17.3 Hz, 2H), 3.24- |
| | diazaspiro[2.5]octan-4- | 2.63 (m, 4H), 2.38-2.15 (m, 2H), 2.15-1.69 (m, |
| | yl)methanone | 4H), 1.35-0.59 (m, 4H). LRMS (ES) m/z 474.6 |
| | | (M⁺ + 1). |
| 342 | (S)-(1-(7-amino-2-(furan-2-yl)- | ¹H NMR (400 MHz, DMSO-d₆) δ 8.39 (d, J = 105.2 |
| | [1,2,4]triazolo[1,5- | Hz, 1H), 7.95-7.77 (m, 1H), 7.30-6.88 (m, 1H), |
| | a][1,3,5]triazin-5-yl)pyrrolidin- | 6.74-6.61 (m, 1H), 4.99 (d, J = 85.7 Hz, 1H), 4.27 |
| | 2-yl)(7-(2,2,2-trifluoroethyl)- | (d, J = 52.1 Hz, 1H), 3.75-3.41 (m, 3H), 3.22-2.61 |
| | 4,7-diazaspiro[2.5]octan-4- | (m, 3H), 2.37-1.72 (m, 6H), 1.20 (d, J = 31.9 Hz, |
| | yl)methanone | 3H), 0.80 (d, J = 50.9 Hz, 2H). LRMS (ES) m/z |
| | | 492.6 (M⁺ + 1). |
| 343 | (S)-(1-(7-amino-2-(furan-2-yl)- | ¹H NMR (400 MHz, DMSO-d₆) δ 8.36 (d, J = 103.5 |
| | [1,2,4]triazolo[1,5- | Hz, 2H), 7.87 (dd, J = 5.0, 1.9 Hz, 1H), 7.13-6.87 |
| | a][1,3,5]triazin-5-yl)pyrrolidin- | (m, 1H), 6.68 (dt, J = 3.6, 1.9 Hz, 1H), 5.28-4.76 |
| | 2-yl)(7-(2,2-difluoropropyl)- | (m, 1H), 4.20 (s, 1H), 3.67 (q, J = 9.8, 8.5 Hz, 2H), |
| | 4,7-diazaspiro[2.5]octan-4- | 3.23-2.57 (m, 5H), 2.29 (dq, J = 12.1, 8.5, 7.9 Hz, |
| | yl)methanone | 2H), 2.14-1.76 (m, 4H), 1.66 (td, J = 19.1, 14.0 Hz, |
| | | 3H), 1.35-1.04 (m, 2H), 1.01-0.60 (m, 2H). |
| | | LRMS (ES) m/z 488.6 (M⁺ + 1). |
| 354 | (S)-2-((7-amino-2-(furan-2- | ¹H NMR (400 MHz, DMSO-d₆) δ 8.22 (d, J = 74.4 |
| | yl)-[1,2,4]triazolo[1,5- | Hz, 2H), 7.87 (dd, J = 1.7, 0.9 Hz, 1H), 7.55 (dd, J = |
| | a][1,3,5]triazin-5-yl)amino)-2- | 19.3, 7.6 Hz, 1H), 7.13-6.99 (m, 1H), 6.68 (dd, J = |
| | cyclopropyl-1-(4-(2,2- | 3.4, 1.8 Hz, 1H), 6.42-6.00 (m, 1H), 4.44 (t, J = |
| | difluoroethyl)piperazin-1- | 7.9 Hz, 1H), 3.59 (q, J = 18.8, 15.5 Hz, 3H), 3.44 (d, |
| | yl)ethan-1-one | J = 13.3 Hz, 1H), 2.86-2.68 (m, 3H), 1.22 (dq, J = |
| | | 11.4, 4.0, 3.2 Hz, 1H), 0.55-0.25 (m, 5H). LRMS |
| | | (ES) m/z 448.6 (M⁺ + 1). |
| 355 | (S)-2-((7-amino-2-(furan-2- | ¹H NMR (400 MHz, DMSO-d₆) δ 8.31 (s, 2H), 7.87 |
| | yl)-[1,2,4]triazolo[1,5- | (s, 1H), 7.43 (dd, J = 51.3, 8.4 Hz, 1H), 7.07 (d, J = |
| | a][1,3,5]triazin-5-yl)amino)-1- | 3.5 Hz, 1H), 6.68 (t, J = 2.5 Hz, 1H), 6.33-5.94 (m, |
| | (4-(2,2- | 2H), 4.78-4.59 (m, 1H), 3.81-3.41 (m, 6H), 2.77 |
| | difluoroethyl)piperazin-1-yl)- | (dp, J = 17.7, 7.5, 6.2 Hz, 4H), 0.93-0.83 (m, 6H). |
| | 3-methylbutan-1-one | LRMS (ES) m/z 450.5 (M⁺ + 1). |
| 356 | (S)-2-((7-amino-2-(furan-2- | ¹H NMR (400 MHz, DMSO-d₆) δ 8.28 (s, 2H), 7.87 |
| | yl)-[1,2,4]triazolo[1,5- | (dd, J = 1.9, 0.9 Hz, 1H), 7.44 (dd, J = 38.4, 7.9 Hz, |
| | a][1,3,5]triazin-5-yl)amino)-1- | 1H), 7.11-7.01 (m, 1H), 6.67 (dd, J = 3.4, 1.8 Hz, |
| | (4-(2,2- | 1H), 4.79 (tt, J = 10.4, 5.3 Hz, 1H), 3.71-3.42 (m, |
| | difluoropropyl)piperazin-1- | 4H), 2.82-2.66 (m, 3H), 1.81-1.54 (m, 5H), 0.95- |
| | yl)butan-1-one | 0.81 (m, 3H). LRMS (ES) m/z 450.1 (M⁺ + 1). |
| 357 | (S)-2-((7-amino-2-(furan-2- | ¹H NMR (400 MHz, DMSO-d₆) δ 8.30 (s, 2H), |
| | yl)-[1,2,4]triazolo[1,5- | 7.91-7.83 (m, 1H), 7.53 (dd, J = 36.2, 7.7 Hz, 1H), |
| | a][1,3,5]triazin-5-yl)amino)-2- | 7.07 (dd, J = 7.0, 3.3 Hz, 1H), 6.68 (dd, J = 3.5, 1.8 |
| | cyclopropyl-1-(4-(2,2- | Hz, 1H), 4.45 (q, J = 10.0, 9.0 Hz, 1H), 3.56 (d, J = |
| | difluoropropyl)piperazin-1- | 52.3 Hz, 4H), 2.76 (dd, J = 17.0, 12.3 Hz, 3H), 1.64 |
| | yl)ethan-1-one | (t, J = 19.1 Hz, 3H), 1.23 (dt, J = 8.4, 5.2 Hz, 1H), |
| | | 0.52-0.29 (m, 4H). LRMS (ES) m/z 462.5 (M⁺ + |
| | | 1). |
| 360 | (S)-2-((7-amino-2-(furan-2- | ¹H NMR (400 MHz, DMSO-d₆) δ 8.27 (d, J = 36.2 |
| | yl)-[1,2,4]triazolo[1,5- | Hz, 2H), 7.87 (d, J = 1.8 Hz, 1H), 7.42 (dd, J = 69.2, |
| | a][1,3,5]triazin-5-yl)amino)-1- | 8.5 Hz, 1H), 7.07 (d, J = 3.3 Hz, 1H), 6.68 (dd, J = |
| | (4-(2,2- | 3.6, 1.9 Hz, 1H), 4.77-4.57 (m, 1H), 3.82-3.41 (m, |
| | difluoropropyl)piperazin-1-yl)- | 4H), 3.18 (d, J = 4.9 Hz, 1H), 2.80-2.61 (m, 3H), |
| | 3-methylbutan-1-one | 2.08 (d, J = 7.4 Hz, 1H), 1.64 (t, J = 19.1 Hz, 3H), |
| | | 0.92 (t, J = 7.2 Hz, 6H). LRMS (ES) m/z 464.5 |
| | | (M⁺ + 1). |
| 358 | (S)-2-((7-amino-2-(furan-2- | ¹H NMR (400 MHz, DMSO-d₆) δ 8.56-7.82 (m, |
| | yl)-[1,2,4]triazolo[1,5- | 2H), 7.54-7.02 (m, 1H), 6.72-6.08 (m, 2H), 4.84- |
| | a][1,3,5]triazin-5-yl)amino)-1- | 4.08 (m, 1H), 3.71-3.41 (m, 4H), 3.03 (td, J = 15.2, |
| | (4-(2,2,3,3- | 8.5 Hz, 2H), 2.82-2.57 (m, 2H), 1.84-1.38 (m, |
| | tetrafluoropropyl)piperazin-1- | 2H), 0.95-0.81 (m, 3H). LRMS (ES) m/z 486.5 |
| | yl)butan-1-one | (M⁺ + 1). |
| 359 | (S)-2-((7-amino-2-(furan-2- | ¹H NMR (400 MHz, DMSO-d₆) δ 8.30 (s,2H), 7.87 |
| | yl)-[1,2,4]triazolo [1,5- | (dd, J = 1.8, 0.8 Hz, 1H), 7.56 (dd, J = 32.7, 7.7 Hz, |
| | a][1,3,5]triazin-5-yl)amino)-2- | 1H), 7.13-7.00 (m, 1H), 6.74-6.36 (m, 2H), 4.43 |
| | cyclopropyl-1-(4-(2,2,3,3- | (q, J = 6.8, 5.6 Hz, 1H), 3.61 (d, J = 20.8 Hz, 4H), |
| | tetrafluoropropyl)piperazin-1- | 3.03 (td, J = 15.3, 7.5 Hz, 2H), 2.55 (s, 4H), 1.30- |
| | yl)ethan-1-one | 1.16 (m, 1H), 0.55-0.28 (m, 4H). LRMS (ES) m/z |
| | | 498.2 (M⁺ + 1). |
| 372 | ((S)-1-(7-amino-2-(furan-2-yl)- | ¹H NMR (400 MHz, DMSO-d₆) δ 8.58-7.99 (m, |
| | [1,2,4]triazolo[1,5- | 2H), 7.93-7.78 (m, 1H), 7.13-6.98 (m, 1H), 6.68 |
| | a][1,3,5]triazin-5-yl)pyrrolidin- | (ddd, J = 6.5, 3.5, 1.8 Hz, 1H), 5.11-4.81 (m, 1H), |
| | 2-yl)((R)-4-(2,2- | 4.33 (d, J = 92.2 Hz, 1H), 4.16-3.77 (m, 1H), 3.64 |
| | difluoropropyl)-2- | (ddd, J = 19.8, 9.8, 4.3 Hz, 2H), 2.98-2.63 (m, |
| | methylpiperazin-1- | 5H), 2.44-2.27 (m, 2H), 2.27-2.05 (m, 1H), 1.97- |
| | yl) methanone | 1.78 (m, 3H), 1.68 (td, J = 19.2, 3.4 Hz, 3H), 1.51 |
| | | (dd, J = 17.7, 6.6 Hz, 2H), 1.15 (d, J = 6.7 Hz, 1H). |
| | | LRMS (ES) m/z 476.3 (M⁺ + 1). |
| 376 | (S)-(1-(5-amino-2-(furan-2-yl)- | ¹H NMR (400 MHz, DMSO-d₆) δ 7.86 (s, 1H), 7.45 |
| | [1,2,4]triazolo[1,5-c]pyrimidin- | (s, 2H), 7.06 (t, J = 3.1 Hz, 1H), 6.67 (dd, J = 3.4, |
| | 7-yl)pyrrolidin-2-yl)(1′-(2,2,2- | 1.8 Hz, 1H), 4.34 (t, J = 15.1 Hz, 1H), 4.05 (s, 1H), |
| | trifluoroethyl)-[4,4′- | 3.48 (s, 2H), 3.11 (dd, J = 14.8, 6.7 Hz, 3H), 2.93 (s, |
| | bipiperidin]-1-yl)methanone | 2H), 2.25 (d, J = 11.3 Hz, 3H), 1.94 (s, 2H), 1.63 (d, |
| | | J = 29.0 Hz, 5H), 1.34-0.93 (m, 6H). LRMS (ES) |
| | | m/z 547.5 (M⁺ + 1). |
| 377 | (S)-(1-(5-amino-2-(furan-2-yl)- | ¹H NMR (400 MHz, DMSO-d₆) δ 7.92-7.78 (m, |
| | [1,2,4]triazolo[1,5-c]pyrimidin- | 1H), 7.62 (s, 2H), 7.06 (dd, J = 3.4, 0.9 Hz, 1H), |
| | 7-yl)azetidin-2-yl)(1′-(2,2,2- | 6.66 (dd, J = 3.4, 1.8 Hz, 1H), 5.46 (d, J = 10.2 Hz, |
| | trifluoroethyl)-[4,4′- | 1H), 5.08-4.96 (m, 1H), 4.38 (t, J = 13.1 Hz, 1H), |
| | bipiperidin]-1-yl)methanone | 3.97-3.79 (m, 2H), 3.70 (d, J = 13.5 Hz, 1H), 3.09 |
| | | (qd, J = 10.3, 9.9, 3.9 Hz, 2H), 3.03-2.83 (m, 3H), |
| | | 2.64 (s, 1H), 2.21 (dq, J = 17.6, 9.6, 8.4 Hz, 3H), 1.75- |
| | | 1.52 (m, 4H), 1.33-0.93 (m, 7H). LRMS (ES) |
| | | m/z 533.5 (M⁺ + 1). |
| 378 | (S)-(1-(5-amino-2-(furan-2-yl)- | ¹H NMR (400 MHz, DMSO-d₆) δ 7.86 (d, J = 1.7 Hz, |
| | [1,2,4]triazolo[1,5-c]pyrimidin- | 1H), 7.63 (s, 2H), 7.06 (d, J = 3.4 Hz, 1H), 6.66 (dd, |
| | 7-yl)azetidin-2-yl)(1′-(2,2- | J = 3.4, 1.7 Hz, 1H), 6.10 (td, J = 55.9, 4.2 Hz, 1H), |
| | difluoroethyl)-[4,4′- | 5.46 (d, J = 9.7 Hz, 1H), 5.05-4.91 (m, 1H), 4.37 |
| | bipiperidin]-1-yl)methanone | (t, J = 12.9 Hz, 1H), 3.97-3.61 (m, 3H), 3.00-2.81 |
| | | (m, 3H), 2.65 (tq, J = 14.3, 4.8 Hz, 3H), 2.49-2.43 |
| | | (m, 1H), 2.24-2.10 (m, 1H), 2.09-1.96 (m, 2H), |
| | | 1.74-1.50 (m, 4H), 1.31-0.89 (m, 6H). LRMS |
| | | (ES) m/z 515.3 (M⁺ + 1). |
| 379 | (S)-(1-(5-amino-2-(furan-2-yl)- | ¹H NMR (400 MHz, DMSO-d₆) δ 7.86 (d, J = 1.7 Hz, |
| | [1,2,4]triazolo[1,5-c]pyrimidin- | 1H), 7.62 (s, 2H), 7.06 (d, J = 3.4 Hz, 1H), 6.66 (dd, |
| | 7-yl)azetidin-2-yl)(1′-(2,2- | J = 3.4, 1.7 Hz, 1H), 5.46 (d, J = 10.2 Hz, 1H), 5.03 |
| | difluoropropyl)-[4,4′- | (dt, J = 9.5, 5.1 Hz, 1H), 4.38 (t, J = 13.1 Hz, 1H), |
| | bipiperidin]-1-yl)methanone | 3.98-3.62 (m, 3H), 3.03-2.80 (m, 3H), 2.64 (td, |
| | | J = 14.0, 4.4 Hz, 3H), 2.13 (dq, J = 40.2, 11.0, 9.5 |
| | | Hz, 3H), 1.74-1.51 (m, 8H), 1.33-0.85 (m, 7H). |
| | | LRMS (ES) m/z 529.5 (M⁺ + 1). |
| 393 | (S)-(1-(5-amino-2-(furan-2-yl)- | ¹H NMR (400 MHz, DMSO-d₆) δ 7.86 (d, J = 1.7 Hz, |
| | [1,2,4]triazolo[1,5-c]pyrimidin- | 1H), 7.69 (ddd, J = 18.3, 5.8, 2.9 Hz, 1H), 7.55-7.37 |
| | 7-yl)pyrrolidin-2-yl)(1′-(2,2- | (m, 1H), 7.05 (d, J = 3.2 Hz, 1H), 6.69-6.58 (m, |
| | difluoroethyl)-[4,4′- | 1H), 6.28-5.86 (m, 1H), 4.41-3.98 (m, 3H), 3.48 |
| | bipiperidin]-1-yl)methanone | (s, 1H), 2.91 (d, J = 10.4 Hz, 2H), 2.68 (tq, J = 12.0, |
| | | 7.0, 5.9 Hz, 2H), 2.22 (d, J = 14.3 Hz, 1H), 2.14- |
| | | 1.88 (m, 4H), 1.86-1.55 (m, 6H), 1.19-1.01 (m, |
| | | 4H), 0.83 (ddd, J = 11.4, 5.3, 2.3 Hz, 3H). LRMS |
| | | (ES) m/z 529.4 (M⁺ + 1). |
| 394 | (S)-(1-(5-amino-2-(furan-2-yl)- | ¹H NMR (400 MHz, DMSO-d₆) δ 7.86 (d, J = 1.7 Hz, |
| | [1,2,4]triazolo[1,5-c]pyrimidin- | 1H), 7.76-7.62 (m, 1H), 7.45 (s, 1H), 7.05 (d, J = |
| | 7-yl)pyrrolidin-2-yl)(1′-(2,2- | 3.3 Hz, 1H), 6.67 (dd, J = 3.3, 1.8 Hz, 1H), 4.43- |
| | difluoropropyl)-[4,4′- | 4.02 (m, 3H), 2.90 (d, J = 10.7 Hz, 2H), 2.67 (dd, J = |
| | bipiperidin]-1-yl)methanone | 14.0, 5.4 Hz, 2H), 2.02 (dt, J = 58.4, 9.6 Hz, 4H), |
| | | 1.72-1.56 (m, 6H), 1.32-1.18 (m, 6H), 0.83 (dtd, |
| | | J = 15.5, 9.3, 7.9, 5.5 Hz, 3H). LRMS (ES) m/z 543.4 |
| | | (M⁺ + 1). |
| 396 | (S)-2-((7-amino-2-(furan-2- | ¹H NMR (400 MHz, DMSO-d₆) δ 8.32 (s, 2H), 7.87 |
| | yl)-[1,2,4]triazolo[1,5- | (d, J = 1.7 Hz, 1H), 7.49 (dd, J = 43-5, 8.2 Hz, 1H), |
| | a][1,3,5]triazin-5-yl)amino)-1- | 7.07 (d, J = 3.4 Hz, 1H), 6.68 (dd, J = 3.5, 1.8 Hz, |
| | (4-(2,2- | 1H), 6.33-6.00 (m, 2H), 5.15-5.02 (m, 1H), 3.70- |
| | difluoroethyl)piperazin-1-yl)- | 3.40 (m, 8H), 3.34 (s, 1H), 3.24 (d, J = 2.4 Hz, |
| | 3-methoxypropan-1-one | 1H), 2.77 (tdd, J = 15.6, 7.3, 4.3 Hz, 3H), 2.70-2.52 |
| | | (m, 3H). LRMS (ES) m/z 452.3 (M⁺ + 1). |
| 397 | (S)-2-((7-amino-2-(furan-2- | ¹H NMR (400 MHz, DMSO-d₆) δ 8.33 (s, 2H), 7.87 |
| | yl)-[1,2,4]triazolo[1,5- | (d, J = 1.7 Hz, 1H), 7.48 (dd, J = 51.5, 8.2 Hz, 1H), |
| | a][1,3,5]triazin-5-yl)amino)-1- | 7.07 (d, J = 3.3 Hz, 1H), 6.68 (dd, J = 3.4, 1.8 Hz, |
| | (4-(2,2- | 1H), 5.10 (dt, J = 8.3, 6.4 Hz, 1H), 3.70-3.40 (m, |
| | difluoropropyl)piperazin-1-yl)- | 8H), 3.34 (s, 1H), 3.24 (d, J = 2.4 Hz, 1H), 2.74 (td, |
| | 3-methoxypropan-1-one | J = 14.0, 4.4 Hz, 2H), 2.67-2.54 (m, 3H), 1.63 (t, |
| | | J = 19.1 Hz, 4H). LRMS (ES) m/z 465.9 (M⁺ + 1). |

### Example 73: Synthesis of compound 73, 1-(4-((7-amino-2-(furan-2-yl)-[1,2,4]triazolo[1,5-a][1,3,5]triazin-5-yl)-L-prolyl)piperazin-1-yl)-3,3,3-trifluoropropan-1-one

### [Step 1] Synthesis of tert-butyl 4-(3,3,3-trifluoropropanoyl)piperazin-1-carboxylate

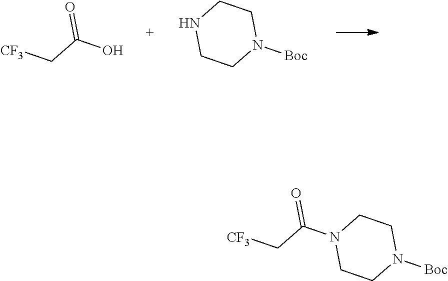 [SMILES: O=C(O)CC(F)(F)F ; CC(C)(C)OC(=O)N1CCNCC1 ; CC(C)(C)OC(=O)N1CCN(C(=O)CC(F)(F)F)CC1]

3,3,3-Trifluoropropanoic acid (0.500 g, 3.905 mmol) and N,N-dimethylformamide (0.003 mL, 0.039 mmol) were dissolved in dichloromethane (10 mL) at room temperature, after which oxalyl dichloride (0.335 mL, 3.905 mmol) was added into the resulting solution and stirred at the same temperature for one hour. To the resulting mixture, a solution obtained by dissolving tert-butyl piperazin-1-carboxylate (0.636 g, 3.413 mmol) and triethylamine (0.951 mL, 6.826 mmol) in dichloromethane (i mL) at room temperature, was added and stirred at the same temperature for one hour. Water was poured into the reaction mixture and an organic layer was extracted with dichloromethane. The organic layer was washed with saturated aqueous solution of ammonium chloride, dehydrated with anhydrous magnesium sulfate, filtered, and concentrated under reduced pressure. An obtained product was used without an additional purification process (title compound, 1.000 g, 98.9%, white solid).

### [Step 2] Synthesis of 3,3,3-trifluoro-1-(piperazin-1-yl)propan-1-one

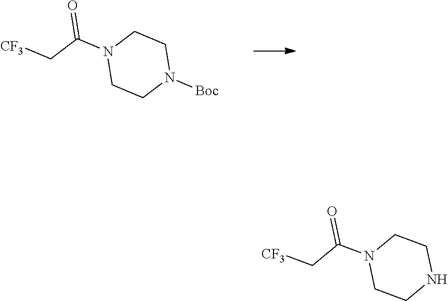 [SMILES: CC(C)(C)OC(=O)N1CCN(C(=O)CC(F)(F)F)CC1 ; O=C(CC(F)(F)F)N1CCNCC1]

Tert-butyl 4-(3,3,3-trifluoropropanoyl)piperazin-1-carboxylate (1.000 g, 3.375 mmol) prepared in step 1 and hydrochloric acid (0.615 g, 16.875 mmol) were dissolved in dichloromethane (10 mL) at room temperature, after which the resulting solution was stirred at the same temperature for 18 hours. Solvent was removed from the reaction mixture under reduced pressure, after which an obtained product was used without an additional purification process (title compound, 0.500 g, 75.5%, white solid).

### [Step 3] Synthesis of tert-butyl (S)-2-(4-(3,3,3-trifluoropropanoyl)piperazin-1-carbonyl)pyrrolidin-1-carboxylate

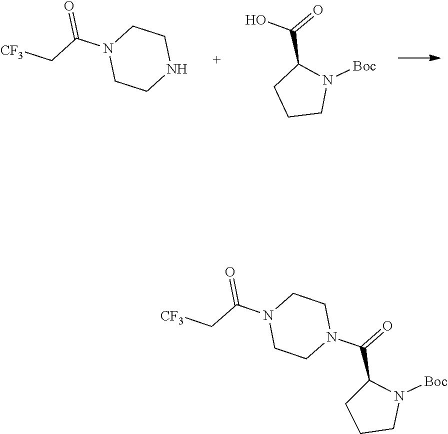 [SMILES: O=C(CC(F)(F)F)N1CCNCC1 ; CC(C)(C)OC(=O)N1CCC[C@H]1C(=O)O ; CC(C)(C)OC(=O)N1CCC[C@H]1C(=O)N1CCN(C(=O)CC(F)(F)F)CC1]

3,3,3-Trifluoro-1-(piperazin-1-yl)propan-1-one (0.500 g, 2.549 mmol) prepared in step 2, (tert-butoxycarbonyl)-L-proline (1.097 g, 5.098 mmol), [dimethylamino(triazolo[4,5-b]pyridin-3-yloxy)methylidene]-dimethylazanium; hexafluorophosphate (1.938 g, 5.098 mmol) and N,N-diisopropylethylamine (1.776 mL, 10.195 mmol) were dissolved in N,N-dimethylformamide (5 mL) at room temperature, after which the resulting solution was stirred at the same temperature for 18 hours. Saturated aqueous solution of ammonium chloride was poured into the reaction mixture, and an organic layer was extracted with ethyl acetate. The organic layer was washed with saturated aqueous solution of sodium hydrogen carbonate, dehydrated with anhydrous sodium sulfate, filtered, and concentrated under reduced pressure. An obtained product was used without an additional purification process (title compound, 0.500 g, 49.9%, brown oil).

### [Step 4] (S)-3,3,3-trifluoro-1-(4-prolylpiperazin-1-yl)propan-1-one

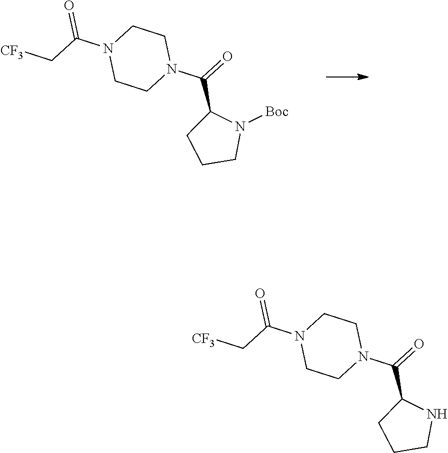 [SMILES: CC(C)(C)OC(=O)N1CCC[C@H]1C(=O)N1CCN(C(=O)CC(F)(F)F)CC1 ; O=C(CC(F)(F)F)N1CCN(C(=O)[C@@H]2CCCN2)CC1]

Tert-butyl (S)-2-(4-(3,3,3-trifluoropropanoyl)piperazin-1-carbonyl)pyrrolidin-1-carboxylate (0.200 g, 0.508 mmol) prepared in step 3 and hydrochloric acid (0.093 g, 2.542 mmol) were dissolved in dichloromethane (5 mL) at room temperature, after which the resulting solution was stirred at the same temperature for 18 hours. Solvent was removed from the reaction mixture under reduced pressure, after which an obtained product was used without an additional purification process (title compound, 0.120 g, 80.5%, brown solid).

### [Step 5] 1-(4-((7-amino-2-(furan-2-yl)-[1,2,4]triazolo[1,5-a][1,3,5]triazin-5-yl)-L-prolyl)piperazin-1-yl)-3,3,3-trifluoropropan-1-one

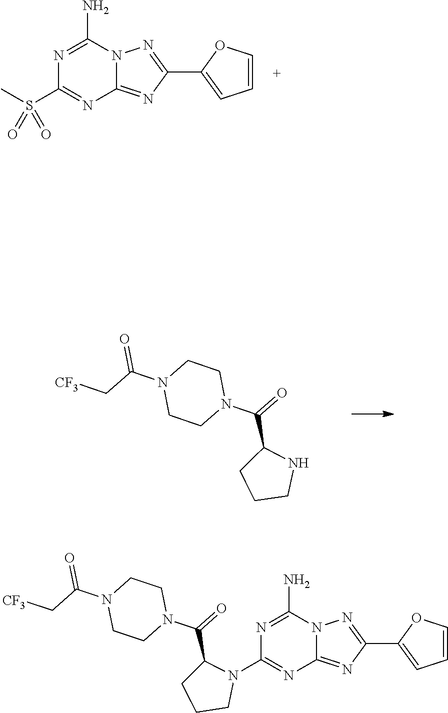 [SMILES: CS(=O)(=O)c1nc(N)n2nc(-c3ccco3)nc2n1 ; O=C(CC(F)(F)F)N1CCN(C(=O)[C@@H]2CCCN2)CC1 ; Nc1nc(N2CCC[C@H]2C(=O)N2CCN(C(=O)CC(F)(F)F)CC2)nc2nc(-c3ccco3)nn12]

2-(Furan-2-yl)-5-(methylsulfonyl)-[1,2,4]triazolo[1,5-a][1,3,5]triazin-7-amine (0.050 g, 0.178 mmol) prepared in step 4, (S)-3,3,3-trifluoro-1-(4-prolylpiperazin-1-yl)propan-1-one (0.105 g, 0.357 mmol) and triethylamine (0.099 mL, 0.714 mmol) were dissolved in dimethylsulfoxide (5 mL) at room temperature, and the resulting solution was stirred at the same temperature for 18 hours. Solvent was removed from the reaction mixture under reduced pressure, after which the resulting concentrate was purified via chromatography (SiO₂ plate, 20×20×1 mm; methanol/ethyl acetate=10%) and concentrated to obtain a title compound (0.020 g, 22.7%) as a yellow solid form.

¹H NMR (400 MHz, DMSO-d₆) δ 8.64-8.09 (m, 1H), 7.87 (ddd, J=5.5, 1.8, 0.8 Hz, 1H), 7.09-7.01 (m, 1H), 6.68 (ddd, J=5.6, 3.4, 1.8 Hz, 1H), 5.09-4.92 (m, 1H), 3.88-3.39 (m, 12H), 2.36-2.19 (m, 1H), 2.04-1.78 (m, 3H); LRMS (ES) m/z 494.5 (M⁺+1).

### Examples 74 to 77, 85 and 86

Example compounds 74 to 77, 85 and 86 were each synthesized through substantially the same synthesis method as a synthesis method of example compound 73 except for using the starting materials shown in the table below instead of 3,3,3-trifluoropropanoic acid in step 1.

TABLE 36

| Example | Starting |
|---|---|
| No. | Materials |
| 74 | 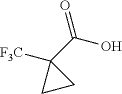 |
| 75 | 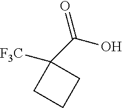 [SMILES: O=C(O)C1(C(F)(F)F)CCC1] |
| 76 | 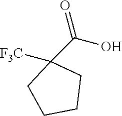 [SMILES: O=C(O)C1(C(F)(F)F)CCCC1] |
| 77 | 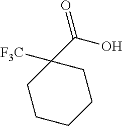 [SMILES: O=C(O)C1(C(F)(F)F)CCCCC1] |
| 85 | 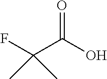 |
| 86 | 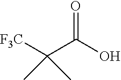 |

Analysis data of each of the compounds prepared as described above are shown in the table below.

TABLE 37

| Example | | |
|---|---|---|
| No. | Compound Names | Analysis Data |
| 74 | 4-((7-amino-2-(furan-2-yl)- | ¹H NMR (400 MHz, DMSO-d₆) δ 8.62-8.10 (m, 1H), |
| | [1,2,4]triazolo[1,5- | 7.87 (dd, J = 5.0, 1.6 Hz, 1H), 7.11-6.97 (m, 1H), |
| | a][1,3,5]triazin-5-yl)-L- | 6.68 (td, J = 3.9, 3.4, 1.7 Hz, 1H), 5.07-4.92 (m, 1H), |
| | prolyl)piperazin-1-yl)(1- | 3.95-3.41 (m, 10H), 2.27 (m, 1H), 2.00-1.80 (m, |
| | (trifluoromethyl)cyclopropyl)m | 3H), 1.43-1.17 (m, 4H); LRMS (ES) m/z 520.5 (M⁺ + |
| | ethanone | 1). |
| 75 | 4-((7-amino-2-(furan-2-yl)- | ¹H NMR (400 MHz, DMSO-d₆) δ 8.19 (m, 2H), 7.87 |
| | [1,2,4]triazolo[1,5- | (ddd, J = 4.6, 1.8, 0.8 Hz, 1H), 7.09-6.98 (m, 1H), |
| | a][1,3,5]triazin-5-yl)-L- | 6.68 (td, J = 3.5, 1.8 Hz, 1H), 4.98 (m, 1H), 3.87- |
| | prolyl)piperazin-1-yl)(1- | 3.21 (m, 10H), 2.51 (m, 4H), 2.26 (m, 1H), 2.05-1.72 |
| | (trifluoromethyl)cyclobutyl)me | (m, 5H); LRMS (ES) m/z 534.5 (M⁺ + 1). |
| | thanone | |
| 76 | 4-((7-amino-2-(furan-2-yl)- | ¹H NMR (400 MHz, DMSO-d₆) δ 8.19 (m, 2H), 7.87 |
| | [1,2,4]triazolo[1,5- | (ddd, J = 5.7, 1.8, 0.8 Hz, 1H), 7.04 (ddd, J = 21.6, |
| | a][1,3,5]triazin-5-yl)-L- | 3.4, 0.8 Hz, 1H), 6.68 (ddd, J = 5.0, 3.4, 1.8 Hz, 1H), |
| | prolyl)piperazin-1-yl)(1- | 5.07-4.91 (m, 1H), 3-92-3-39 (m, 10H), 2.51-1.74 |
| | (trifluoromethyl)cyclopentyl)m | (m, 8H), 1.69-1.53 (m, 4H); LRMS (ES) m/z 548.5 |
| | ethanone | (M⁺ + 1). |
| 77 | 4-((7-amino-2-(furan-2-yl)- | ¹H NMR (400 MHz, DMSO-d₆) δ 8.60-8.11 (m, 2H), |
| | [1,2,4]triazolo[1,5- | 7.90-7.83 (m, 1H), 7.04 (ddd, J = 24.0, 3.4, 0.8 Hz, |
| | a][1,3,5]triazin-5-yl)-L- | 1H), 6.68 (ddd, J = 5.0, 3.4, 1.8 Hz, 1H), 4.98 (m, 1H), |
| | prolyl)piperazin-1-yl)(1- | 3.98-3.32 (m, 10H), 2.26 (m, 1H), 2.01-1.80 (m, |
| | (trifluoromethyl)cyclohexyl)me | 4H), 1.74-1.45 (m, 5H), 1.41-1.19 (m, 4H); LRMS |
| | thanone | (ES) m/z 562.6 (M⁺ + 1). |
| 85 | 1-(4-((7-amino-2-(furan-2-yl)- | ¹H NMR (400 MHz, DMSO-d₆) δ 8.59-8.12 (m, |
| | [1,2,4]triazolo[1,5- | 2H), 7.87 (ddd, J = 4.9, 1.8, 0.8 Hz, 1H), 7.05 (ddd, J |
| | a][1,3,5]triazin-5-yl)-L- | = 15.3, 3.4, 0.8 Hz, 1H), 6.68 (ddd, J = 6.1, 3.4, 1.8 |
| | prolyl)piperazin-1-yl)-2-fluoro- | Hz, 1H), 5.06-4.94 (m, 1H), 3.95-3.43 (m, 10H), |
| | 2-methylpropan-1-one | 2.34-2.17 (m, 1H), 2.03-1.78 (m, 3H), 1.59 (m, |
| | | 6H); LRMS (ES) m/z 472.5 (M⁺ + 1). |
| 86 | 1-(4-((7-amino-2-(furan-2-yl)- | ¹H NMR (400 MHz, DMSO-d₆) δ 8.18 (m, 2H), 7.87 |
| | [1,2,4]triazolo[1,5- | (ddd, J = 5.1, 1.8, 0.8 Hz, 1H), 7.04 (ddd, J = 18.9, |
| | a][1,3,5]triazin-5-yl)-L- | 3.4, 0.9 Hz, 1H), 6.68 (ddd, J = 5.2, 3.4, 1.8 Hz, 1H), |
| | prolyl)piperazin-1-yl)-3,3,3- | 5.06-4.94 (m, 1H), 3.92-3.39 (m, 10H), 2.35-2.19 |
| | trifluoro-2,2-dimethylpropan- | (m, 1H), 2.08-1.80 (m, 2H), 1.52 (d, J = 1.9 Hz, 6H); |
| | 1-one | LRMS (ES) m/z 522.5 (M⁺ + 1). |

### Example 56: Synthesis of Compound 56, (S)-(1-(7-amino-2-(furan-2-yl)-[1,2,4]triazolo[1,5-a][1,3,5]triazin-5-yl)pyrrolidin-2-yl)(4-(4,4-difluorocyclohexyl)piperazin-1-yl)methanone

### [Step 1] Synthesis of tert-butyl 4-(4,4-difluorocyclohexyl)piperazin-1-carboxylate

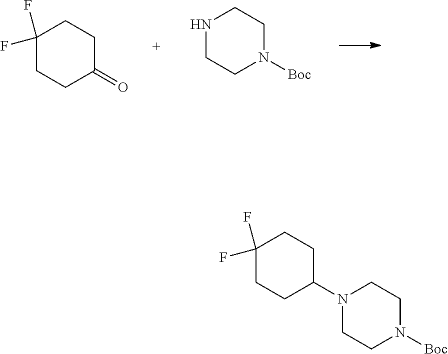 [SMILES: O=C1CCC(F)(F)CC1 ; CC(C)(C)OC(=O)N1CCNCC1 ; CC(C)(C)OC(=O)N1CCN(C2CCC(F)(F)CC2)CC1]

4,4-Difluorocyclohexan-1-one (0.402 g, 3.000 mmol), tert-butyl piperazin-1-carboxylate (0.559 g, 3.000 mmol) and sodium triacetoxyborohydride (0.954 g, 4.500 mmol) were dissolved in dichloromethane (10 mL) at room temperature, after which the resulting solution was stirred at the same temperature for two hours. Saturated aqueous solution of sodium hydrogen carbonate was poured into the reaction mixture, and then an organic layer was extracted with dichloromethane, filtered via a plastic filter to remove a solid residue and an aqueous solution layer therefrom, and concentrated under reduced pressure. An obtained product was used without an additional purification process (title compound, 0.910 g, 99.7%, light yellow oil).

### [Step 2] Synthesis of 1-(4,4-difluorocyclohexyl)piperazine hydrochloride

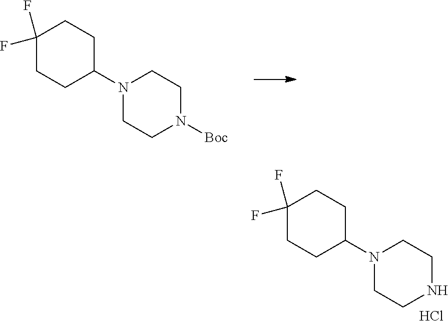 [SMILES: CC(C)(C)OC(=O)N1CCN(C2CCC(F)(F)CC2)CC1 ; FC1(F)CCC(N2CCNCC2)CC1 ; Cl]

Tert-butyl 4-(4,4-difluorocyclohexyl)piperazin-1-carboxylate (0.910 g, 2.990 mmol) prepared in step 1 and hydrogen chloride (4.00 M solution in 1,4-dioxane, 2.990 mL, 11.959 mmol) were dissolved in dichloromethane (5 mL) at room temperature, after which the resulting solution was stirred at the same temperature for five hours. A precipitated solid was filtered, washed with dichloromethane, and dried to obtain a title compound (0.710 g, 98.7%) as a white solid form.

### [Step 3] Synthesis of tert-butyl (S)-2-(4-(4,4-difluorocyclohexyl)piperazin-1-carbonyl)pyrrolidin-1-carboxylate

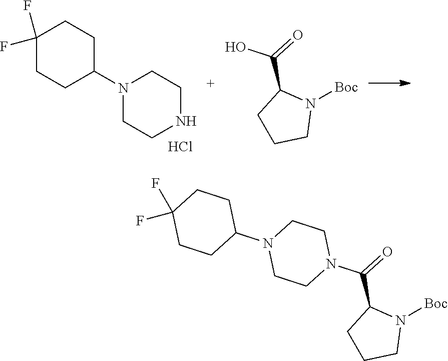

1-(4,4-Difluorocyclohexyl)piperazine hydrochloride (0.710 g, 2.949 mmol) prepared in step 2, (tert-butoxycarbonyl)-L-proline (0.698 g, 3.244 mmol), 1-ethyl-3-(3-dimethylaminopropyl)carbodiimide hydrochloride (EDC-HCl, 1.131 g, 5.899 mmol), 1H-benzo[d][1,2,3]triazol-1-ol (HOBt, 0.399 g, 2.949 mmol) and N,N-diisopropylethylamine (1.541 mL, 8.848 mmol) were dissolved in dichloromethane (10 mL) at room temperature, after which the resulting solution was stirred at the same temperature for 18 hours. Water was poured into the reaction mixture, and then an organic layer was extracted with dichloromethane, filtered via a plastic filter to remove a solid residue and an aqueous solution layer therefrom, and concentrated under reduced pressure. An obtained product was used without an additional purification process (title compound, 1.180 g, 99.6%, light yellow oil).

### [Step 4] Synthesis of (S)-1-(4,4-difluorocyclohexyl)-4-prolylpiperazine hydrochloride

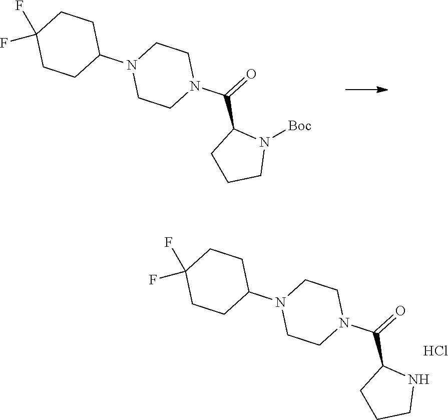 [SMILES: CC(C)(C)OC(=O)N1CCC[C@H]1C(=O)N1CCN(C2CCC(F)(F)CC2)CC1 ; O=C([C@@H]1CCCN1)N1CCN(C2CCC(F)(F)CC2)CC1 ; Cl]

Tert-butyl (S)-2-(4-(4,4-difluorocyclohexyl)piperazin-1-carbonyl)pyrrolidin-1-carboxylate (1.180 g, 2.939 mmol) prepared in step 3 and hydrogen chloride (4.00 M solution in 1,4-dioxane, 2.939 mL, 11.756 mmol) were dissolved in dichloromethane (5 mL) at room temperature, after which the resulting solution was stirred at the same temperature for three hours. Solvent was removed from the reaction mixture under reduced pressure, after which an obtained product was used without an additional purification process (title compound, 0.990 g, 99.7%, light yellow solid).

### [Step 5] Synthesis of (S)-(1-(7-amino-2-(furan-2-yl)-[1,2,4]triazolo[1,5-a][1,3,5]triazin-5-yl)pyrrolidin-2-yl)(4-(4,4-difluorocyclohexyl)piperazin-1-yl)methanone

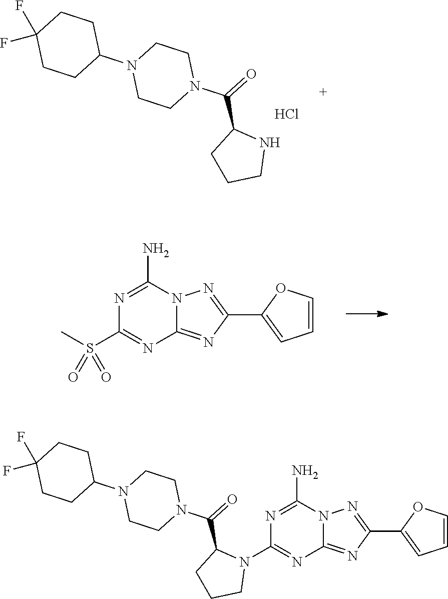 [SMILES: O=C([C@@H]1CCCN1)N1CCN(C2CCC(F)(F)CC2)CC1 ; Cl ; CS(=O)(=O)c1nc(N)n2nc(-c3ccco3)nc2n1 ; Nc1nc(N2CCC[C@H]2C(=O)N2CCN(C3CCC(F)(F)CC3)CC2)nc2nc(-c3ccco3)nn12]

(S)-1-(4,4-Difluorocyclohexyl)-4-prolylpiperazine hydrochloride (0.338 g, 1.000 mmol) prepared in step 4, 2-(furan-2-yl)-5-(methylsulfonyl)-[1,2,4]triazolo[1,5-a][1,3,5]triazin-7-amine (0.280 g, 1.000 mmol) and triethylamine (0.418 mL, 3.000 mmol) were dissolved in N,N-dimethylformamide (3 mL) at room temperature, after which the resulting solution was stirred at the same temperature for 18 hours. Water was poured into the reaction mixture, and then an organic layer was extracted with dichloromethane, filtered via a plastic filter to remove a solid residue and an aqueous solution layer therefrom, and concentrated under reduced pressure. The resulting concentrate was purified via column chromatography (SiO₂, 12 g cartridge; methanol/dichloromethane=0 to 10%) and concentrated to obtain a product, after which dichloromethane (2 mL) was inserted into the obtained product, stirred to filter out a precipitated solid, washed with hexane, and dried to obtain a title compound (0.131 g, 26.0%) as a white solid form.

¹H NMR (400 MHz, DMSO-d₆) δ 8.61-7.99 (m, 2H), 7.92-7.82 (m, 1H), 7.10-6.98 (m, 1H), 6.73-6.61 (m, 1H), 5.16-4.91 (m, 1H), 3.99-3.53 (m, 5H), 3.51-3.36 (m, 2H), 2.95-2.65 (m, 1H), 2.44-2.15 (m, 3H), 2.15-1.70 (m, 9H), 1.66-1.44 (m, 2H), 1.39-1.16 (m, 1H); LRMS (ES) m/z 502.6 (M⁺+1).

### Examples 152 to 156, 392 and 403

Example compounds 152 to 156, 392 and 403 were each synthesized through substantially the same synthesis method as a synthesis method of example compound 56 except for using the starting materials shown in the table below instead of 4,4-difluorocyclohexan-1-one of step 1 in a synthesis method of example compound 56.

TABLE 38

| Example | Starting |
|---|---|
| No. | Materials |
| 152 | 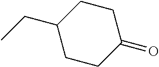 |
| 153 | 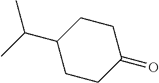 |
| 154 | 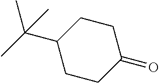 |
| 155 | 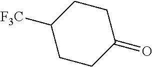 [SMILES: O=C1CCC(C(F)(F)F)CC1] |
| 156 | 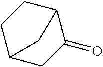 |
| 392 | 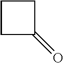 |
| 403 | 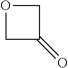 |

### Example 344: Synthesis of Compound 344

Example compound 344 was synthesized through substantially the same synthesis method as a synthesis method of example compound 56 except for using cyclohexanone instead of 4,4-difluorocyclohexan-1-one of step 1 and using tert-butyl 4,7-diazaspiro[2.5]octan-4-carboxylate instead of tert-butyl piperazin-1-carboxylate.

### Example 398: Synthesis of Compound 398

Example compound 398 was synthesized through substantially the same synthesis method as a synthesis method of example compound 56 except for using N-(tert-butoxycarbonyl)-O-methyl-L-serine instead of (tert-butoxycarbonyl)-L-proline.

### Example 404: Synthesis of Compound 404

Example compound 404 was synthesized through substantially the same synthesis method as a synthesis method of example compound 403 except for using 2-(furan-2-yl)-7-(methylsulfonyl)-[1,2,4]triazolo[1,5-c]pyrimidin-5-amine instead of 2-(furan-2-yl)-5-(methylsulfonyl)-[1,2,4]triazolo[1,5-a][1,3,5]triazin-7-amine.

### Example 291: Synthesis of Compound 291

Example compound 291 was synthesized through substantially the same synthesis method as a synthesis method of example compound 56 except for using 4,4,4-trifluorobutanal instead of 4,4-difluorocyclohexan-1-one of step and using tert-butyl [4,4′-bipiperidin]-1-carboxylate instead of tert-butyl piperazin-1-carboxylate.

Analysis data of each of the compounds prepared as described above are shown in the table below.

TABLE 39

| Example | | |
|---|---|---|
| No. | Compound Names | Analysis Data |
| 152 | (S)-(1-(7-amino-2-(furan-2- | ¹H NMR (400 MHz, DMSO-d₆) δ 8.68-7.99 (m, |
| | yl)-[1,2,4]triazolo[1,5- | 2H), 7.90-7.82 (m, 1H), 7.09-7.00 (m, 1H), 6.70- |
| | a][1,3,5]triazin-5- | 6.62 (m, 1H), 5.06-4.90 (m, 1H), 3.86-3.45 |
| | yl)pyrrolidin-2-yl)(4-(4- | (m, 5H), 3.28-3.12 (m, 1H), 2.87-2.64 (m, 1H), |
| | ethylcyclohexyl)piperazin-1- | 2.62-2.52 (m, 1H), 2.45-2.14 (m, 4H), 2.03- |
| | yl) methanone | 1.72 (m, 4H), 1.72-1.33 (m, 7H), 1.33-1.13 (m, |
| | | 3H), 0.86 (t, J = 7.3 Hz, 3H); LRMS (ES) m/z 494.7 |
| | | (M⁺ + 1). |
| 153 | (S)-(1-(7-amino-2-(furan-2- | ¹H NMR (400 MHz, DMSO-d₆) δ 8.71-7.96 (m, |
| | yl)-[1,2,4]triazolo[1,5- | 2H), 7.91-7.80 (m, 1H), 7.13-6.96 (m, 1H), 6.73- |
| | a][1,3,5]triazin-5- | 6.56 (m, 1H), 5.10-4.90 (m, 1H), 3.78-3.44 (m, |
| | yl)pyrrolidin-2-yl)(4-(4- | 5H), 2.83-2.57 (m, 1H), 2.45-2.12 (m, 4H), 2.00- |
| | isopropylcyclohexyl)piperazi | 1.64 (m, 6H), 1.61-1.29 (m, 6H), 1.28-0.92 (m, |
| | n-1-yl)methanone | 3H), 0.91-0.76 (m, 6H); LRMS (ES) m/z 508.7 |
| | | (M⁺ + 1). |
| 154 | (S)-(1-(7-amino-2-(furan-2- | ¹H NMR (400 MHz, DMSO-d₆) δ 8.63-7.98 (m, |
| | yl)-[1,2,4]triazolo[1,5- | 2H), 7.91-7.81 (m, 1H), 7.11-6.99 (m, 1H), 6.72- |
| | a][1,3,5]triazin-5- | 6.61 (m, 1H), 5.08-4.90 (m, 1H), 3.82-3.45 (m, |
| | yl)pyrrolidin-2-yl)(4-(4-(tert- | 5H), 3.25 (d, J = 12.0 Hz, 1H), 2.81-2.64 (m, 1H), |
| | butyl)cyclohexyl)piperazin-1- | 2.63-2.55 (m, 1H), 2.40-2.09 (m, 4H), 2.03 (d, |
| | yl)methanone | J = 11.3 Hz, 2H), 1.97-1.72 (m, 3H), 1.49-1.18 (m, |
| | | 6H), 1.17-0.98 (m, 1H), 0.85 (d, J = 2.2 Hz, 9H); |
| | | LRMS (ES) m/z 522.7 (M⁺ + 1). |
| 155 | (S)-(1-(7-amino-2-(furan-2- | ¹H NMR (400 MHz, DMSO-d₆) δ 8.69-7.97 (m, |
| | yl)-[1,2,4]triazolo[1,5- | 2H), 7.91-7.83 (m, 1H), 7.11-7.00 (m, 1H), 6.73- |
| | a][1,3,5]triazin-5- | 6.62 (m, 1H), 5.08-4.91 (m, 1H), 3.84-3.45 (m, |
| | yl)pyrrolidin-2-yl)(4-(4- | 5H), 3.30-3.14 (m, 1H), 2.81-2.54 (m, 2H), 2.43- |
| | (trifluoromethyl)cyclohexyl)p | 2.09 (m, 5H), 2.05-1.76 (m, 5H), 1.76-1.61 (m, |
| | iperazin-1-yl)methanone | 2H), 1.61-1.36 (m, 4H); LRMS (ES) m/z 534.6 |
| | | (M⁺ + 1). |
| 156 | ((S)-1-(7-amino-2-(furan-2- | ¹H NMR (400 MHz, DMSO-d₆) δ 8.69-7.96 (m, |
| | yl)-[1,2,4]triazolo[1,5- | 2H), 7.92-7.81 (m, 1H), 7.10-7.00 (m, 1H), 6.73- |
| | a][1,3,5]triazin-5- | 6.61 (m, 1H), 5.07-4.89 (m, 1H), 3.84-3.43 (m, |
| | yl)pyrrolidin-2-yl)(4- | 5H), 2.73-2.54 (m, 1H), 2.47-2.36 (m, 1H), 2.36- |
| | (bicyclo[2.2.1]heptan-2- | 2.00 (m, 6H), 1.99-1.57 (m, 5H), 1.56-1.40 (m, |
| | yl)piperazin-1-yl)methanone | 1H), 1.40-1.08 (m, 5H), 0.97-0.76 (m, 1H); |
| | | LRMS (ES) m/z 478.7 (M⁺ + 1). |
| 392 | (S)-(1-(7-amino-2-(furan-2- | ¹H NMR (400 MHz, DMSO-d₆) δ 8.67-7.99 (m, |
| | yl)-[1,2,4]triazolo[1,5- | 2H), 7.91-7.84 (m, 1H), 7.09-7.02 (m, 1H), 6.71- |
| | a][1,3,5]triazin-5- | 6.65 (m, 1H), 4.99 (td, J = 9.2, 2.9 Hz, 1H), 3.73- |
| | yl)pyrrolidin-2-yl)(4- | 3.51 (m, 5H), 3.31-3.18 (m, 2H), 2.83-2.70 (m, |
| | cyclobutylpiperazin-1- | 1H), 2.42-2.22 (m, 2H), 2.22-1.73 (m, 9H), 1.72- |
| | yl)methanone | 1.55 (m, 2H); LRMS (ES) m/z 438.3 (M⁺ + 1). |
| 403 | (S)-(1-(7-amino-2-(furan-2- | ¹H NMR (400 MHz, DMSO-d₆) δ 8.59-8.01 (m, |
| | yl)-[1,2,4]triazolo[1,5- | 2H), 7.87 (s, 1H), 7.09-7.02 (m, 1H), 6.68 (dd, J = |
| | a][1,3,5]triazin-5- | 3.3, 1.8 Hz, 1H), 5.05-4.93 (m, 1H), 4.64-4.40 |
| | yl)pyrrolidin-2-yl)(4-(oxetan- | (m, 4H), 3.75-3.39 (m, 7H), 2.69-2.54 (m, 1H), |
| | 3-yl)piperazin-1- | 2.42-2.06 (m, 4H), 1.98-1.78 (m, 3H); LRMS |
| | yl)methanone | (ES) m/z 440.1 (M⁺ + 1). |
| 404 | (S)-(1-(5-amino-2-(furan-2- | ¹H NMR (400 MHz, DMSO-d₆) δ 7.87 (s, 1H), 7.70- |
| | yl)-[1,2,4]triazolo[1,5- | 7.30 (m, 2H), 7.09-7.01 (m, 1H), 6.70-6.64 (m, |
| | clpyrimidin-7-yl)pyrrolidine- | 1H), 5.64 (brs, 1H), 4.99 (brs, 1H), 4.56 (t, J = 6.4 |
| | 2-yl)(4-(oxetan-3- | Hz, 2H), 4.47 (t, J = 5.9 Hz, 2H), 3.76-3.40 (m, |
| | yl)piperazin-1-yl)methanone | 6H), 2.40-2.04 (m, 6H), 2.03-1.72 (m, 3H); |
| | | LRMS (ES) m/z 439.1 (M⁺ + 1). |
| 291 | (S)-(1-(7-amino-2-(furan-2- | ¹H NMR (400 MHz, DMSO-d₆) δ 8.28 (d, J = 138.2 |
| | yl)-[1,2,4]triazolo[1,5- | Hz, 2H), 7.87 (d, J = 4.3 Hz, 1H), 7.09-6.98 (m, |
| | a][1,3,5]triazin-5- | 1H), 6.67 (dq, J = 5.1, 3.1 Hz, 1H), 5.07-4.94 (m, |
| | yl)pyrrolidin-2-yl)(1′-(4,4,4- | 1H), 4.35 (t, J = 14.8 Hz, 1H), 4.09 (q, J = 16.9, 16.1 |
| | trifluorobutyl)-[4,4′- | Hz, 1H), 3.64 (tq, J = 18.8, 7.2, 5.5 Hz, 2H), 3.21- |
| | bipiperidin]-1-yl)methanone | 2.97 (m, 1H), 2.87 (d, J = 10.9 Hz, 2H), 2.38-2.16 |
| | | (m, 5H), 1.97-1.60 (m, 11H), 1.38-0.91 (m, 6H); |
| | | LRMS (ES) m/z 576.6 (M⁺ + 1). |
| 344 | (S)-(1-(7-amino-2-(furan-2- | ¹H NMR (400 MHz, DMSO-d₆) δ 8.46 (d, J = 116.2 |
| | yl)-[1,2,4]triazolo[1,5- | Hz, 1H), 7.88 (d, J = 2.2 Hz, 1H), 7.00 (d, J = 3.4 |
| | a][1,3,5]triazin-5- | Hz, 1H), 6.73-6.62 (m, 1H), 4.44 (d, J = 13.6 Hz, |
| | yl)pyrrolidin-2-yl)(7- | 1H), 3.66 (dq, J = 27.9, 6.7 Hz, 3H), 3.23-3.07 (m, |
| | cyclohexyl-4,7- | 2H), 2.40-2.13 (m, 3H), 1.88 (d, J = 29.2 Hz, 7H), |
| | diazaspiro[2.5]octan-4- | 1.39 (d, J = 31.9 Hz, 4H), 1.25 (dd, J = 10.2, 4.9 Hz, |
| | yl)methanone | 7H); LRMS (ES) m/z 492.7 (M⁺ + 1). |
| 398 | (S)-2-((7-amino-2-(furan-2- | ¹H NMR (400 MHz, DMSO-d₆) δ 8.27 (d, J = 49.1 |
| | yl)-[1,2,4]triazolo[1,5- | Hz, 2H), 7.87 (d, J = 1.7 Hz, 1H), 7.47 (dd, J = 36.4, |
| | a][1,3,5]triazin-5-yl)amino)- | 8.2 Hz, 1H), 7.07 (dd, J = 7.3, 3.4 Hz, 1H), 6.68 (dd, |
| | 1-(4-(4,4- | J = 3.5, 1.8 Hz, 1H), 5.14-5.01 (m, 1H), 3.69-3.38 |
| | difluorocyclohexyl)piperazin- | (m, 7H), 3.34 (s, 1H), 2.66-2.54 (m, 1H), 2.43 (q, |
| | 1-yl)-3-methoxypropan-1-one | J = 7.8, 5.0 Hz, 3H), 2.00 (t, J = 10.8 Hz, 2H), 1.89- |
| | | 1.69 (m, 5H), 1.61-1.45 (m, 2H); LRMS (ES) m/z |
| | | 506.4 (M⁺ + 1). |

### Example 129: Synthesis of corm pound 129, (2S)-2-((7-amino-2-(furan-2-yl)-[1,2,4]triazolo[1,5-a][1,3,5]triazin-5-yl)amino)-1-(3,5-dimethyl-4-(morpholine-4-carbonyl)piperazin-1-yl)propan-1-one

### [Step 1] Synthesis of tert-butyl 3,5-dimethyl-4-(morpholine-4-carbonyl)piperazin-1-carboxylate

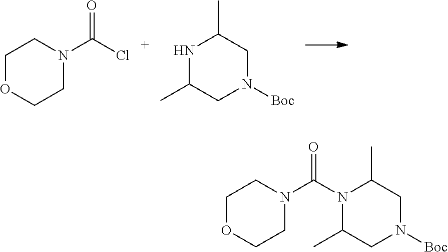 [SMILES: O=C(Cl)N1CCOCC1 ; CC1CN(C(=O)OC(C)(C)C)CC(C)N1 ; CC1CN(C(=O)OC(C)(C)C)CC(C)N1C(=O)N1CCOCC1]

Tert-butyl 3,5-dimethylpiperazin-1-carboxylate (0.321 g, 1.500 mmol), N,N-diisopropylethylamine (0.261 mL, 1.500 mmol) and morpholin-4-carbonyl chloride (0.175 mL, 1.500 mmol) were dissolved in acetonitrile (5 mL) at room temperature, after which the resulting solution was stirred at the same temperature for 18 hours. Saturated aqueous solution of sodium hydrogen carbonate was poured into the reaction mixture, and then an organic layer was extracted with dichloromethane, filtered via a plastic filter to remove a solid residue and an aqueous solution layer therefrom, and concentrated under reduced pressure. The resulting concentrate was purified via column chromatography (SiO₂, 12 g cartridge; methanol/dichloromethane=0 to 5%) and concentrated to obtain a title compound (0.205 g, 41.7%) as a white solid form.

### [Step 2] Synthesis of (2,6-dimethylpiperazin-1-yl)(morpholino)methanone hydrochloride

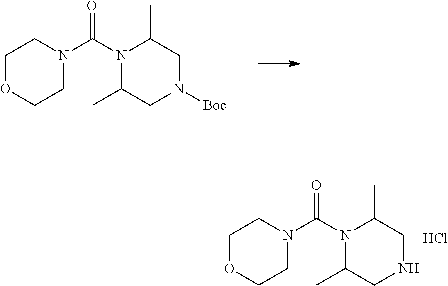 [SMILES: CC1CN(C(=O)OC(C)(C)C)CC(C)N1C(=O)N1CCOCC1 ; CC1CNCC(C)N1C(=O)N1CCOCC1 ; Cl]

Tert-butyl 3,5-dimethyl-4-(morpholine-4-carbonyl)piperazin-1-carboxylate (0.205 g, 0.626 mmol) prepared in step 1 and hydrogen chloride (4.00 M solution in 1,4-dioxane, 0.626 mL, 2.504 mmol) were dissolved in dichloromethane (3 mL) at room temperature, after which the resulting solution was stirred at the same temperature for 18 hours. Solvent was removed from the reaction mixture under reduced pressure, after which an obtained product was used without an additional purification process (title compound, 0.165 g, 99.9%, white solid).

### [Step 3] Synthesis of tert-butyl ((2S)-1-(3,5-dimethyl-4-(morpholine-4-carbonyl)piperazin-1-yl)-1-oxopropan-2-yl)carbamate

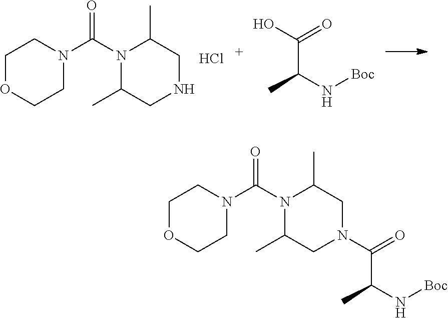 [SMILES: CC1CNCC(C)N1C(=O)N1CCOCC1 ; Cl ; C[C@H](NC(=O)OC(C)(C)C)C(=O)O ; CC1CN(C(=O)[C@H](C)NC(=O)OC(C)(C)C)CC(C)N1C(=O)N1CCOCC1]

(2,6-Dimethylpiperazin-1-yl)(morpholino)methanone hydrochloride (0.165 g, 0.626 mmol) prepared in step 2, (tert-butoxycarbonyl)-L-alanine (0.130 g, 0.688 mmol), triethylamine (0.218 mL, 1.564 mmol) and 2,4,6-tripropyl-1,3,5,2,4,6-trioxatriphosphinane 2,4,6-trioxide (T3P, 50.00% solution in EtOAc, 0.558 mL, 0.938 mmol) were dissolved in dichloromethane (5 mL) at room temperature, after which the resulting solution was stirred at 40° C. for 18 hours to complete the reaction by lowering a temperature to room temperature. Saturated aqueous solution of sodium hydrogen carbonate was poured into the reaction mixture, and then an organic layer was extracted with dichloromethane, filtered via a plastic filter to remove a solid residue and an aqueous solution layer therefrom, and then concentrated under reduced pressure. An obtained product was used without an additional purification process (title compound, 0.249 g, 99.9%, light yellow oil).

### [Step 4] Synthesis of (2S)-2-amino-1-(3,5-dimethyl-4-(morpholine-4-carbonyl)piperazin-1-yl)propan-1-one hydrochloride

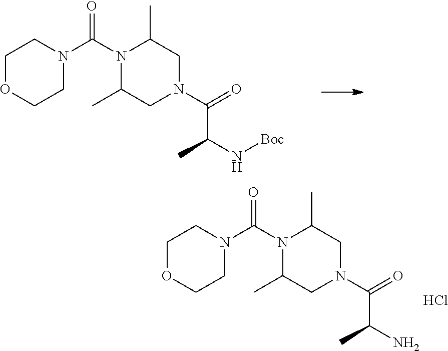

Tert-butyl ((2S)-1-(3,5-dimethyl-4-(morpholine-4-carbonyl)piperazin-1-yl)-1-oxopropan-2-yl)carbamate (0.249 g, 0.625 mmol) prepared in step 3 and hydrogen chloride (4.00 M solution in 1,4-dioxane, 0.625 mL, 2.499 mmol) were dissolved in dichloromethane (3 mL) at room temperature, after which the resulting solution was stirred at the same temperature for 18 hours. Solvent was removed from the reaction mixture under reduced pressure, after which an obtained product was used without an additional purification process (title compound, 0.209 g, 99.9%, white solid).

### [Step 5] Synthesis of (2S)-2-((7-amino-2-(furan-2-yl)-[1,2,4]triazolo[1,5-a][1,3,5]triazin-5-yl)amino)-1-(3,5-dimethyl-4-(morpholine-4-carbonyl)piperazin-1-yl)propan-1-one

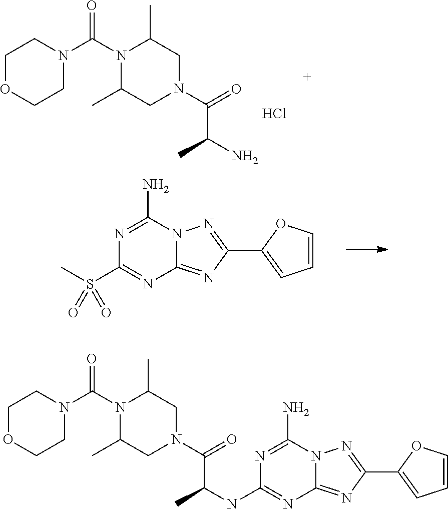

(2S)-2-Amino-1-(3,5-dimethyl-4-(morpholine-4-carbonyl)piperazin-1-yl)propan-1-one hydrochloride (0.209 g, 0.624 mmol) prepared in step 4, 2-(furan-2-yl)-5-(methylsulfonyl)-[1,2,4]triazolo[1,5-a][1,3,5]triazin-7-amine (0.175 g, 0.624 mmol) and sodium hydrogen carbonate (0.210 g, 2.497 mmol) were dissolved in cyclopentyl methyl ether (CPME, 4 mL) at room temperature, after which the resulting solution was stirred at 50° C. for 18 hours to complete the reaction by lowering a temperature to room temperature. Solvent was removed from the reaction mixture under reduced pressure, after which the resulting concentrate was purified via column chromatography (SiO₂, 12 g cartridge; methanol/dichloromethane=0 to 5%) and concentrated to obtain a title compound (0.069 g, 22.0%) as a white solid form.

¹H NMR (400 MHz, DMSO-d₆) δ 8.49-7.98 (m, 2H), 7.90-7.82 (m, 1H), 7.77-7.42 (m, 1H), 7.06 (dd, J=13.0, 3.5 Hz, 1H), 6.71-6.63 (m, 1H), 5.04-4.75 (m, 1H), 3.89-3.36 (m, 11H), 3.29-2.88 (m, 3H), 1.36-1.24 (m, 3H), 1.22-0.89 (m, 6H); LRMS (ES) m/z 499.6 (M⁺+1).

### Example 179: Synthesis of Compound 179

Example compound 179 was synthesized through substantially the same synthesis method as a synthesis method of example compound 129 except for using (tert-butoxycarbonyl)-L-proline instead of (tert-butoxycarbonyl)-L-alanine and using tert-butyl 3,8-diazabicyclo[3.2.1]octan-3-carboxylate and phenylcarbamic chloride instead of tert-butyl 3,5-dimethylpiperazin-1-carboxylate and morpholine-4-carbonyl chloride, respectively.

### Examples 180 to 184

Example compounds 180 to 184 were each synthesized through substantially the same synthesis method as a synthesis method of example compound 179 except for using the compounds of the following table instead of tert-butyl 3,8-diazabicyclo[3.2.1]octan-3-carboxylate as a starting material.

TABLE 40

| Example No. | Starting Materials |
|---|---|
| 180 | 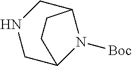 [SMILES: CC(C)(C)OC(=O)N1C2CCC1CNC2] |
| 181 | 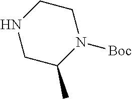 |
| 182 | 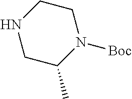 |
| 183 | 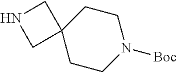 [SMILES: CC(C)(C)OC(=O)N1CCC2(CC1)CNC2] |
| 184 | 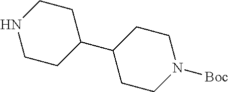 [SMILES: CC(C)(C)OC(=O)N1CCC(C2CCNCC2)CC1] |

Analysis data of each of the compounds prepared as described above are shown in the table below.

TABLE 41

| Example | | |
|---|---|---|
| No. | Compound Names | Analysis Data |
| 179 | 3-((7-amino-2-(furan-2-yl)- | ¹H NMR (400 MHz, DMSO-d₆) δ 8.80-8.62 (m, |
| | [1,2,4]triazolo[1,5- | 1H), 8.32 (d, J = 108.7 Hz, 2H), 7.90-7.82 (m, 1H), |
| | a][1,3,5]triazin-5-yl)-L-prolyl)- | 7.54 (tt, J = 6.4, 1.6 Hz, 2H), 7.30-7.21 (m, 2H), |
| | N-phenyl-3,8- | 7.09-7.03 (m, 1H), 6.95 (td, J = 7.3, 1.2 Hz, 1H), |
| | diazabicyclo[3.2.1]octane-8- | 6.71-6.62 (m, 1H), 5.16-4.82 (m, 1H), 4.50 (td, J = |
| | carboxamide | 22.8, 21.9, 6.8 Hz, 2H), 4.15-3.98 (m, 1H), 3.95- |
| | | 3.76 (m, 1H), 3.74-3.50 (m, 3H), 2.88 (dd, J = |
| | | 25.2, 12.6 Hz, 1H), 2.46-2.35 (m, 1H), 2.19 (tt, J = |
| | | 12.2, 6.0 Hz, 1H), 2.05-1.79 (m, 5H), 1.71-1.50 |
| | | (m, 2H). LRMS (ES) m/z 529.6 (M⁺ + 1). |
| 180 | 8-((7-amino-2-(furan-2-yl)- | ¹H NMR (400 MHz, DMSO-d₆) δ 8.45 (d, J = 12.9 |
| | [1,2,4]triazolo[1,5- | Hz, 1H), 8.15 (s, 1H), 7.90-7.65 (m, 1H), 7.56- |
| | a][1,3,5]triazin-5-yl)-L-prolyl)- | 7.43 (m, 2H), 7.29-7.19 (m, 2H), 7.09-6.86 (m, |
| | N-phenyl-3,8- | 2H), 6.71-6.48 (m, 1H), 4.98-4.78 (m, 1H), 4.62- |
| | diazabicyclo[3.2.1]octane-3- | 4.45 (m, 2H), 4.19-3-77 (m, 3H), 3.65 (dd, J = |
| | carboxamide | 15.4, 7.6 Hz, 2H), 3.09-2.88 (m, 1H), 2.29 (d, J = |
| | | 13.7 Hz, 1H), 2.10-1.59 (m, 7H), 1.27 (dd, J = 16.2, |
| | | 9.8 Hz, 2H), 0.92-0.82 (m, 1H); LRMS (ES) m/z |
| | | 529.6 (M⁺ + 1). |
| 181 | (S)-4-((7-amino-2-(furan-2- | ¹H NMR (400 MHz, DMSO-d₆) δ 8.63-8.52 (m, |
| | yl)-[1,2,4]triazolo [1,5- | 1H), 8.14 (d, J = 14.8 Hz, 1H), 7.89-7.84 (m, 1H), |
| | a][1,3,5]triazine-5-yl)-L- | 7.49 (d, J = 7.9 Hz, 2H), 7.30-7.21 (m, 2H), 7.06 |
| | prolyl)-3-methyl-N- | (dq, J = 8.2, 3.4 Hz, 1H), 6.96 (td, J = 7.3, 1.6 Hz, |
| | phenylpiperazin-1- | 1H), 6.70-6.62 (m, 1H), 5.15-4.86 (m, 1H), 4.73- |
| | carboxamide | 4.42 (m, 1H), 4.25-3.81 (m, 3H), 3.74-3.50 (m, |
| | | 3H), 3.10-2.90 (m, 2H), 2.29 (q, J = 5.8 Hz, 1H), |
| | | 1.95 (dddd, J = 24.9, 19.0, 12.7, 7.6 Hz, 3H), 1.31- |
| | | 1.21 (m, 1H), 1.10 (t, J = 6.1 Hz, 2H). LRMS (ES) |
| | | m/z 517.7 (M⁺ + 1). |
| 182 | (R)-4-((7-amino-2-(furan-2- | ¹H NMR (400 MHz, DMSO-d₆) δ 8.71-7.98 (m, |
| | yl)-[1,2,4]triazolo[1,5- | 3H), 7.98-7.60 (m, 1H), 7.48 (d, J = 7.9 Hz, 2H), |
| | a][1,3,5]triazine-5-yl)-L- | 7.25 (t, J = 7.9 Hz, 2H), 7.14-6.84 (m, 2H), 6.67 |
| | prolyl)-3-methyl-N- | (d, J = 11.1 Hz, 1H), 5.04 (d, J = 33.0 Hz, 1H), 4.42 |
| | phenylpiperazin-1- | (d, J = 84.0 Hz, 1H), 4.25-3.87 (m, 3H), 3.76- |
| | carboxamide | 3.53 (m, 3H), 3.25-2.78 (m, 2H), 2.41-2.17 (m, |
| | | 1H), 1.85 (d, J = 60.6 Hz, 3H), 1.53-1.17 (m, 2H), |
| | | 0.96 (d, J = 98.2 Hz, 2H). LRMS (ES) m/z 517.7 |
| | | (M⁺ + 1). |
| 183 | 7-((7-amino-2-(furan-2-yl)- | ¹H NMR (400 MHz, Chloroform-d) δ 8.41 (s, 1H), |
| | [1,2,4]triazolo[1,5- | 7.60-7.48 (m, 1H), 7.35 (t, J = 8.6 Hz, 1H), 7.27- |
| | a][1,3,5]triazin-5-yl)-L-prolyl)- | 7.20 (m, 1H), 7.15-6.98 (m, 4H), 6.90-6.83 (m, |
| | N-phenyl-2,7- | 1H), 6.67 (s, 1H), 6.53 (dd, J = 3.5, 1.8 Hz, 1H), 4.96 |
| | diazaspiro[3.5]nonane-2- | (dd, J = 8.5, 3.9 Hz, 1H), 4.45 (d, J = 13.7 Hz, 1H), |
| | carboxamide | 3.89 (td, J = 13.2, 12.7, 6.6 Hz, 3H), 3.81-3.72 (m, |
| | | 4H), 3.63-3.50 (m, 1H), 3.33-3.24 (m, 1H), 2.79 |
| | | (t, J = 13.0 Hz, 1H), 2.37-2.29 (m, 1H), 2.20 (d, J = |
| | | 2.3 Hz, 1H), 2.03 (d, J = 15.1 Hz, 3H), 1.83-1.56 |
| | | (m, 5H); LRMS (ES) m/z 543.6 (M⁺ + 1). |
| 184 | 1′-((7-amino-2-(furan-2-yl)- | ¹H NMR (400 MHz, DMSO-d₆) δ 8.50 (d, J = 3.8 |
| | [1,2,4]triazolo[1,5- | Hz, 3H), 7.91-7.82 (m, 1H), 7.48 (d, J = 8.0 Hz, |
| | a][1,3,5]triazin-5-yl)-L-prolyl)- | 2H), 7.21 (t, J = 7.8 Hz, 2H), 7.10-7.00 (m, 1H), |
| | N-phenyl-[4,4′-bipiperidine]-1- | 6.91 (t, J = 7.3 Hz, 1H), 6.67 (dt, J = 3.6, 2.0 Hz, |
| | carboxamide | 1H), 5.06-4.91 (m, 1H), 4.35 (t, J = 14.3 Hz, 1H), |
| | | 4.26-3.93 (m, 3H), 3.61 (dq, J = 24.0, 6.9 Hz, 3H), |
| | | 3.20-2.89 (m, 2H), 2.80-2.61 (m, 2H), 2.35- |
| | | 2.15 (m, 1H), 1.91 (dt, J = 13.2, 6.2 Hz, 2H), 1.76 (d, |
| | | J = 48.4 Hz, 6H), 1.26 (d, J = 7.6 Hz, 8H), 1.18- |
| | | 0.92 (m, 4H); LRMS (ES) m/z 585.7 (M⁺ + 1). |

### Example 43: Synthesis of compound 43, (S)-(1-(7-amino-2-(furan-2-yl)-[1,2,4]triazolo[1,5-a][1,3,5]triazin-5-yl)pyrrolidin-2-yl)(4-(2-ethyl-2-fluorobutyl)piperazin-1-yl)methanone

### [Step 1] Synthesis of tert-butyl 4-(2-ethyl-2-fluorobutyl)piperazin-1-carboxylate

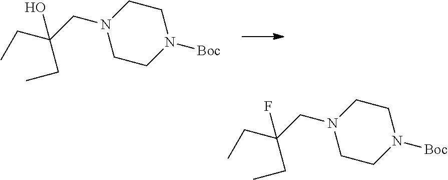 [SMILES: CCC(O)(CC)CN1CCN(C(=O)OC(C)(C)C)CC1 ; CCC(F)(CC)CN1CCN(C(=O)OC(C)(C)C)CC1]

Tert-butyl 4-(2-ethyl-2-hydroxybutyl)piperazin-1-carboxylate (3.000 g, 10.474 mmol) was dissolved in dichloromethane (300 mL), after which DAST (2.076 mL, 15.711 mmol) was added thereinto at 0° C., then stirred at the same temperature for 30 minutes, and then further stirred at room temperature for 18 hours. Saturated aqueous solution of sodium hydrogen carbonate was poured into the reaction mixture, and an organic layer was extracted with dichloromethane. The organic layer was washed with saturated aqueous solution of sodium chloride, dehydrated with anhydrous magnesium sulfate, filtered, and concentrated under reduced pressure. The resulting concentrate was purified via column chromatography (SiO₂, 40 g cartridge; ethyl acetate/hexane=0 to 25%) and concentrated to obtain a title compound (0.500 g, 16.6%) as a colorless oil form.

### [Step 2] Synthesis of 1-(2-ethyl-2-fluorobutyl)piperazine

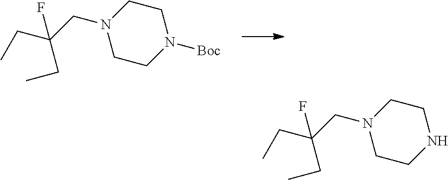 [SMILES: CCC(F)(CC)CN1CCN(C(=O)OC(C)(C)C)CC1 ; CCC(F)(CC)CN1CCNCC1]

Tert-butyl 4-(2-ethyl-2-fluorobutyl)piperazin-1-carboxylate (0.500 g, 1.734 mmol) prepared in step 1 and hydrochloric acid (4.00 M solution in dioxane, 4.334 mL, 17.336 mmol) were dissolved in dichloromethane (10 mL) at room temperature, after which the resulting solution was stirred at the same temperature for 18 hours. Solvent was removed from the reaction mixture under reduced pressure, after which an obtained product was used without an additional purification process (title compound, 0.320 g, 98.0%, brown solid).

### [Step 3] Tert-butyl (S)-2-(4-(2-ethyl-2-fluorobutyl)piperazin-1-carbonyl)pyrrolidin-1-carboxylate

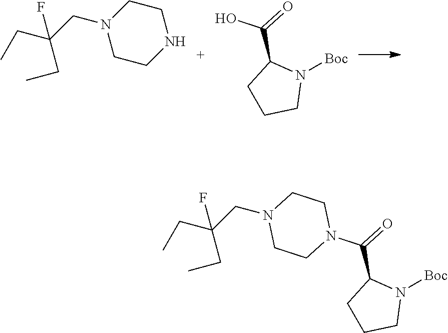 [SMILES: CCC(F)(CC)CN1CCNCC1 ; CC(C)(C)OC(=O)N1CCC[C@H]1C(=O)O ; CCC(F)(CC)CN1CCN(C(=O)[C@@H]2CCCN2C(=O)OC(C)(C)C)CC1]

1-(2-Ethyl-2-fluorobutyl)piperazine (0.500 g, 2.655 mmol) prepared in step 2, (tert-butoxycarbonyl)-L-proline (1.143 g, 5.311 mmol), [dimethylamino(triazolo[4,5-b]pyridin-3-yloxy)methylidene]-dimethylazanium; hexafluorophosphate (2.019 g, 5.311 mmol) and N,N-diisopropylethylamine (0.925 mL, 5.311 mmol) were dissolved in N,N-dimethylformamide (15 mL) at room temperature, after which the resulting solution was stirred at the same temperature for 18 hours. Water was poured into the reaction mixture and an organic layer was extracted with ethyl acetate. The organic layer was washed with saturated aqueous solution of sodium chloride, dehydrated with anhydrous magnesium sulfate, filtered, and concentrated under reduced pressure. The resulting concentrate was purified via column chromatography (SiO₂, 12 g cartridge; methanol/dichloromethane=0 to 10%) and concentrated to obtain a title compound (0.200 g, 19.5%) as a white solid form.

### [Step 4] Synthesis of (S)-1-(2-ethyl-2-fluorobutyl)-4-prolylpiperazine

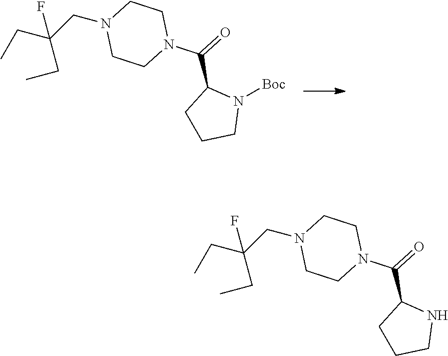 [SMILES: CCC(F)(CC)CN1CCN(C(=O)[C@@H]2CCCN2C(=O)OC(C)(C)C)CC1 ; CCC(F)(CC)CN1CCN(C(=O)[C@@H]2CCCN2)CC1]

Tert-butyl (S)-2-(4-(2-ethyl-2-fluorobutyl)piperazin-1-carbonyl)pyrrolidin-1-carboxylate (0.200 g, 0.519 mmol) prepared in step 3 and hydrochloric acid (4.00 M solution in dioxane, 1.297 mL, 5.188 mmol) were dissolved in dichloromethane (10 mL) at room temperature, after which the resulting solution was stirred at the same temperature for 18 hours. Solvent was removed from the reaction mixture under reduced pressure, after which an obtained product was used without an additional purification process (title compound, 0.140 g, 94.6%, white solid).

### [Step 5] (S)-(1-(7-amino-2-(furan-2-yl)-[1,2,4]triazolo[1,5-a][1,3,5]triazin-5-yl)pyrrolidin-2-yl)(4-(2-ethyl-2-fluorobutyl)piperazin-1-yl)methanone

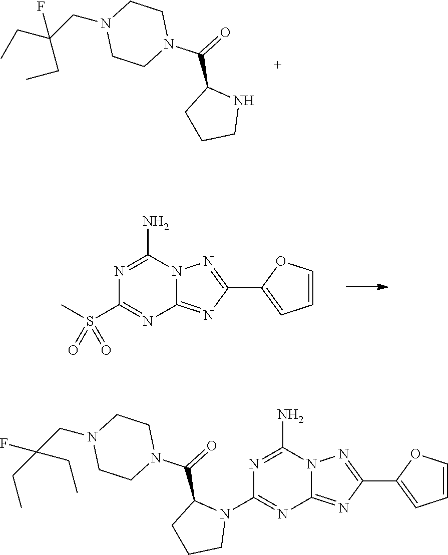 [SMILES: CCC(F)(CC)CN1CCN(C(=O)[C@@H]2CCCN2)CC1 ; CS(=O)(=O)c1nc(N)n2nc(-c3ccco3)nc2n1 ; CCC(F)(CC)CN1CCN(C(=O)[C@@H]2CCCN2c2nc(N)n3nc(-c4ccco4)nc3n2)CC1]

(S)-1-(2-Ethyl-2-fluorobutyl)-4-prolylpiperazine (0.150 g, 0.526 mmol) prepared in step 4, 2-(furan-2-yl)-5-(methylsulfonyl)-[1,2,4]triazolo[1,5-a][1,3,5]triazin-7-amine (0.074 g, 0.263 mmol) and triethylamine (0.147 mL, 1.051 mmol) were dissolved in dimethylsulfoxide (5 mL) at room temperature, after which the resulting solution was stirred at the same temperature for 18 hours. Water was poured into the reaction mixture and an organic layer was extracted with ethyl acetate. The organic layer was washed with saturated aqueous solution of sodium chloride, dehydrated with anhydrous magnesium sulfate, filtered, and concentrated under reduced pressure. The resulting concentrate was purified via chromatography (SiO₂ plate, 20×20×1 mm; methanol/dichloromethane=10%) and concentrated to obtain a title compound (0.005 g, 2.0%) as a white solid form.

¹H NMR (400 MHz, DMSO-d₆) δ 8.64-8.08 (m, 2H), 7.87 (s, 1H), 7.09-7.01 (m, 1H), 6.68 (s, 1H), 5.05-4.92 (m, 1H), 3.77-3.36 (m, 6H), 2.81-2.21 (m, 6H), 2.03-1.58 (m, 8H), 0.91-0.79 (m, 6H); LRMS (ES) m/z 486.5 (M⁺+1).

### Example 245: Synthesis of Compound 245

Example compound 245 was synthesized through substantially the same synthesis method as a synthesis method of example compound 43 except for using tert-butyl 1′-(2-ethyl-2-hydroxybutyl)-[4,4′-bipiperidine]-1-carboxylate instead of tert-butyl 4-(2-ethyl-2-hydroxybutyl)piperazin-1-carboxylate of step 1.

### Examples 322 and 332

Example compounds 322 and 332 were each synthesized through substantially the same synthesis method as a synthesis method of example compound 245 except for using (S)-1-(tert-butoxycarbonyl)azetidin-2-carboxylic acid and (tert-butoxycarbonyl)-L-alanine, respectively, instead of (tert-butoxycarbonyl)-L-proline.

### Example 374: Synthesis of Compound 374

Example compound 374 was synthesized through substantially the same synthesis method as a synthesis method of example compound 43 except for using tert-butyl (R)-4-(2-hydroxy-2-methylpropyl)-2-methylpiperazin-1-carboxylate instead of tert-butyl 4-(2-ethyl-2-hydroxybutyl)piperazin-1-carboxylate of step 1.

### Example 382: Synthesis of Compound 382

Example compound 382 was synthesized through substantially the same synthesis method as a synthesis method of example compound 322 except for using 2-(furan-2-yl)-7-(methylsulfonyl)-[1,2,4]triazolo[1,5-c]pyrimidin-5-amine instead of 2-(furan-2-yl)-5-(methylsulfonyl)-[1,2,4]triazolo[1,5-a][1,3,5]triazin-7-amine.

### Example 395: Synthesis of Compound 395

Example compound 395 was synthesized through substantially the same synthesis method as a synthesis method of example compound 245 except for using 2-(furan-2-yl)-7-(methylsulfonyl)-[1,2,4]triazolo[1,5-c]pyrimidin-5-amine instead of 2-(furan-2-yl)-5-(methylsulfonyl)-[1,2,4]triazolo[1,5-a][1,3,5]triazin-7-amine.

Analysis data of each of the compounds prepared as described above are shown in the table below.

TABLE 42

| Example | | |
|---|---|---|
| No. | Compound Names | Analysis Data |
| 245 | (S)-(1-(7-amino- | ¹H NMR (400 MHz, DMSO-d₆) |
| | 2-(furan-2- | δ 8.58-8.04 (m, 3H), |
| | yl)-[1,2,4]triazolo[1,5- | 7.87 (s, 1H), 7.09-6.94 (m, 1H), |
| | a][1,3,5]triazin-5- | 6.68 (s, 1H), 5.00 (d, J = |
| | yl)pyrrolidin-2-yl)(1′-(2- | 11.5 Hz, 1H), 4.43-3.97 |
| | fluoro-2-methylpropyl)- | (m, 3H), 3.63 (dd, J = 24.0, |
| | [4,4′-bipiperidin]-1- | 10.6 Hz, 2H), 2.93 (s, 2H), |
| | yl)methanone | 2.17-1.56 (m, 12H), 1.38- |
| | | 1.00 (m, 13H). LRMS (ES) |
| | | m/z 540.5 (M⁺ + 1). |
| 322 | (S)-(1-(7-amino- | ¹H NMR (400 MHz, DMSO-d₆) |
| | 2-(furan-2- | δ 8.63-8.12 (m, 2H), |
| | yl)-[1,2,4]triazolo[1,5- | 7.87 (d, J = 1.8 Hz, 1H), 7.12-6.94 |
| | a][1,3,5]triazin-5- | (m, 1H), 6.68 (dd, J = |
| | yl)azetidin-2-yl)(1′-(2- | 3.4, 1.8 Hz, 1H), 5.21 (d, |
| | fluoro-2-methylpropyl)- | J = 7.9 Hz, 1H), 4.47-3.70 |
| | [4,4′-bipiperidin]-1- | (m, 5H), 2.98 (d, J = 47.2 Hz, |
| | yl) methanone | 3H), 2.72-2.59 (m, 1H), |
| | | 2.46-2.32 (m, 2H), 2.15-1.90 |
| | | (m, 3H), 1.65 (dd, J = |
| | | 39.6, 12.9 Hz, 4H), 1.49-1.13 |
| | | (m, 11H), 1.00 (d, J = 12.4 |
| | | Hz, 3H), 0.85 (dd, J = 7.1, 2.0 |
| | | Hz, 1H). LRMS (ES) m/z |
| | | 526.3 (M⁺ + 1). |
| 332 | (S)-2-((7- | ¹H NMR (400 MHz, DMSO-d₆) |
| | amino-2-(furan-2- | δ 8.25 (s, 2H), 7.89- |
| | yl)-[1,2,4]triazolo[1,5- | 7.79 (m, 1H), 7.37 (dd, J = 20.9, |
| | a][1,3,5]triazin- | 7.4 Hz, 1H), 7.12-6.99 |
| | 5-yl)amino)- | (m, 1H), 6.67 (dd, J = 3.4, |
| | 1-(1′-(2-fluoro-2- | 1.8 Hz, 1H), 4.94-4.83 (m, |
| | methylpropyl)-[4,4′- | 1H), 4.40 (d, J = 12.8 Hz, 1H), |
| | bipiperidin]- | 4.01 (d, J = 13.8 Hz, 1H), |
| | 1-yl)propan-1- | 3.02 (q, J = 13.3, 12.0 Hz, 1H), |
| | one | 2.90 (d, J = 10.8 Hz, 2H), |
| | | 2.37 (d, J = 22.8 Hz, 2H), |
| | | 2.06-1.93 (m, 2H), 1.82- |
| | | 1.53 (m, 5H), 1.37-1.13 (m, |
| | | 14H), 1.03 (t, J = 15.7 Hz, |
| | | 2H). LRMS (ES) m/z 514.6 |
| | | (M⁺ + 1). |
| 374 | ((S)-1-(7-amino- | ¹H NMR (400 MHz, DMSO-d₆) |
| | 2-(furan-2- | δ 8.55-7.94 (m, 2H), |
| | yl)-[1,2,4]triazolo[1,5- | 7.87 (ddd, J = 5.4, 1.8, 0.8 Hz, |
| | a][1,3,5]triazin-5- | 1H), 7.06 (ddd, J = 4.3, |
| | yl)pyrrolidin- | 2.7, 0.9 Hz, 1H), 6.68 (ddd, |
| | 2-yl)((R)-4-(2- | J = 6.3, 3.4, 1.8 Hz, 1H), |
| | fluoro-2- | 5.06-4.83 (m, 1H), 4.30 (d, |
| | methylpropyl)-2- | J = 92.8 Hz, 1H), 4.12- |
| | methylpiperazin-1- | 3.74 (m, 1H), 3.72-3.54 (m, |
| | yl) methanone | 2H), 3.01-2.69 (m, 3H), |
| | | 2.49-2.15 (m, 4H), 2.06-1.70 |
| | | (m, 4H), 1.54 (dd, J = |
| | | 17.5, 6.6 Hz, 2H), 1.36 (dt, |
| | | J = 21.4, 2.5 Hz, 6H), 1.17 (d, |
| | | J = 6.7 Hz, 1H). LRMS (ES) |
| | | m/z 472.4 (M⁺ + 1). |
| 382 | (S)-(1-(5-amino- | ¹H NMR (400 MHz, DMSO-d₆) |
| | 2-(furan-2- | δ 7.86 (d, J = 1.7 Hz, |
| | yl)-[1,2,4]triazolo[1,5- | 1H), 7.61 (s, 2H), 7.05 (d, |
| | c]pyrimidin-7- | J = 3.4 Hz, 1H), 6.67 (dd, J = |
| | yl)azetidin-2- | 3.4, 1.7 Hz, 1H), 5.46 (d, |
| | yl)(1′-(2-fluoro-2- | J = 10.0 Hz, 1H), 5.11-5.01 |
| | methylpropyl)-[4,4′- | (m, 1H), 4.39 (t, J = 13.6 Hz, |
| | bipiperidin]- | 1H), 4.00-3.57 (m, 3H), |
| | 1-yl)methanone | 2.96 (d, J = 42.0 Hz, 3H), 2.66 |
| | | (d, J = 10.1 Hz, 1H), 2.38 |
| | | (dd, J = 23.0, 4.2 Hz, 2H), |
| | | 2.25-2.11 (m, 1H), 2.00 (q, |
| | | J = 10.5 Hz, 2H), 1.77-1.55 |
| | | (m, 4H), 1.37-1.14 (m, |
| | | 10H), 1.12-0.94 (m, 3H). |
| | | LRMS (ES) m/z 525.4 (M⁺ + |
| | | 1). |
| 395 | (S)-(1-(5-amino- | ¹H NMR (400 MHz, DMSO-d₆) |
| | 2-(furan-2- | δ 7.86 (d, J = 1.7 Hz, |
| | yl)-[1,2,4]triazolo[1,5- | 1H), 7.44 (s, 1H), 7.05 (d, |
| | c]pyrimidin- | J = 3.2 Hz, 1H), 6.66 (dd, J = |
| | 7-yl)pyrrolidin- | 3-4, 1.8 Hz, 1H), 4.34 (t, J = |
| | 2-yl)(1′-(2-fluoro-2- | 14.9 Hz, 1H), 4.06 (s, 1H), |
| | methylpropyl)-[4,4′- | 3.47 (s, 1H), 2.90 (d, J = |
| | bipiperidin]- | 11.2 Hz, 2H), 2.38 (dd, J = |
| | 1-yl)methanone | 23.0, 5.5 Hz, 2H), 2.24 (s, 1H), |
| | | 1.98 (p, J = 12.7, 11.9 Hz, |
| | | 4H), 1.85-1.55 (m, 5H), |
| | | 1.42-1.14 (m, 10H), 1.00 (td, |
| | | J = 24.9, 23.9, 12.5 Hz, 3H), |
| | | 0.83 (ddd, J = 11.6, 6.8, 2.4 |
| | | Hz, 1H). LRMS (ES) |
| | | m/z 539.5 (M⁺ + 1). |

### Example 282: Synthesis of Compound 282

### [Step 1] Synthesis of tert-butyl 4-(3,3,3-trifluoro-2,2-dimethylpropanoyl)piperazin-1-carboxylate

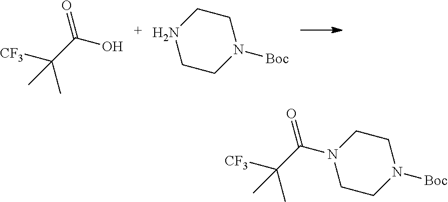

3,3,3-Trifluoro-2,2-dimethylpropanoic acid (1.000 g, 6.406 mmol), oxalyl chloride (0.550 mL, 6.406 mmol) and N,N-dimethylformamide (0.049 mL, 0.641 mmol) were dissolved in dichloromethane (10 mL) at room temperature, after which the resulting solution was stirred at the same temperature for 18 hours. To the resulting mixture, a solution obtained by dissolving tert-butyl piperazin-1-carboxylate (1.067 g, 5.729 mmol) and triethylamine (1.597 mL, 11.458 mmol) in dichloromethane (5 mL) at room temperature, was added and stirred at the same temperature for 18 hours. Solvent was removed from the reaction mixture under reduced pressure, after which saturated aqueous solution of ammonium chloride was poured into the resulting concentrate and an organic layer was extracted with ethyl acetate. The organic layer was washed with saturated aqueous solution of sodium chloride, dehydrated with anhydrous magnesium sulfate, filtered, and concentrated under reduced pressure. An obtained product was used without an additional purification process (title compound, 1.800 g, 96.9%, white oil).

### [Step 2] Synthesis of tert-butyl 4-((1-(trifluoromethyl)cyclobutyl)methyl)piperazin-1-carboxylate

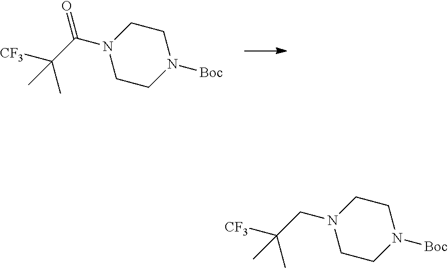 [SMILES: CC(C)(C)OC(=O)N1CCN(C(=O)C(C)(C)C(F)(F)F)CC1 ; CC(C)(C)OC(=O)N1CCN(CC(C)(C)C(F)(F)F)CC1]

Tert-butyl 4-(1-(trifluoromethyl)cyclobutan-1-carbonyl)piperazin-1-carboxylate (2.000 g, 6.166 mmol) prepared in step 2, trifluoroborane (45.00% solution in Et₂O, 4.647 mL, 30.832 mmol) and sodium borohydride (0.467 g, 12.333 mmol) were dissolved in tetrahydrofuran (20 mL) at room temperature, after which the resulting solution was stirred at the same temperature for 18 hours. Solvent was removed from the reaction mixture under reduced pressure, after which saturated aqueous solution of sodium hydrogen carbonate was poured into the resulting concentrate, and an organic layer was extracted with ethyl acetate. The organic layer was washed with saturated aqueous solution of sodium chloride, dehydrated with anhydrous magnesium sulfate, filtered, and concentrated under reduced pressure. An obtained product was used without an additional purification process (title compound, 1.500 g, 78.4%, brown oil).

### [Step 3] 1-(3,3,3-trifluoro-2,2-dimethylpropyl)piperazine

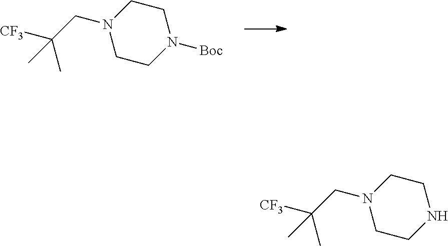 [SMILES: CC(C)(C)OC(=O)N1CCN(CC(C)(C)C(F)(F)F)CC1 ; CC(C)(CN1CCNCC1)C(F)(F)F]

Tert-butyl 4-(3,3,3-trifluoro-2,2-dimethylpropyl)piperazin-1-carboxylate (1.500 g, 4.833 mmol) prepared in step 2 and hydrochloric acid (4.00 M solution in dioxane, 12.083 mL, 48.331 mmol) were dissolved in dichloromethane (20 mL) at room temperature, after which the resulting solution was stirred at the same temperature for 18 hours. Solvent was removed from the reaction mixture under reduced pressure, after which an obtained product was used without an additional purification process (title compound, 0.900 g, 88.6%, brown oil).

### [Step 4] Tert-butyl (S)-2-(4-(3,3,3-trifluoro-2,2-dimethylpropyl)piperazin-1-carbonyl)pyrrolidin-1-carboxylate

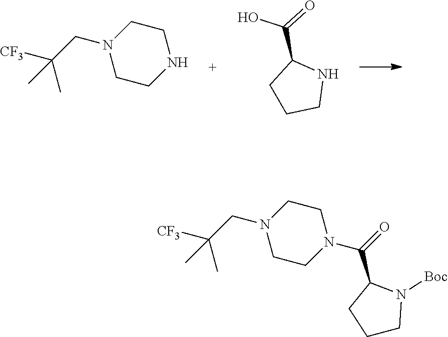 [SMILES: CC(C)(CN1CCNCC1)C(F)(F)F ; O=C(O)[C@@H]1CCCN1 ; CC(C)(C)OC(=O)N1CCC[C@H]1C(=O)N1CCN(CC(C)(C)C(F)(F)F)CC1]

1-(3,3,3-Trifluoro-2,2-dimethylpropyl)piperazine (1.000 g, 4.756 mmol) prepared in step 3, L-proline (1.095 g, 9.513 mmol), 1-[bis(dimethylamino)methylene]-1H-1,2,3-triazolo[4,5-b]pyridinium 3-oxide hexafluorophosphate (HATU, 3.617 g, 9.513 mmol) and N,N-diisopropylethylamine (4.142 mL, 23.782 mmol) were dissolved in dichloromethane (20 mL) at room temperature, after which the resulting solution was stirred at the same temperature for 18 hours. Solvent was removed from the reaction mixture under reduced pressure, after which saturated aqueous solution of sodium hydrogen carbonate was poured into the resulting concentrate, and an organic layer was extracted with ethyl acetate. The organic layer was washed with saturated aqueous solution of sodium hydrogen carbonate, dehydrated with anhydrous magnesium sulfate, filtered, and concentrated under reduced pressure. An obtained product was used without an additional purification process (title compound, 1.500 g, 77.4%, brown oil).

### [Step 5] (S)-1-prolyl-4-(3,3,3-trifluoro-2,2-dimethylpropyl)piperazine

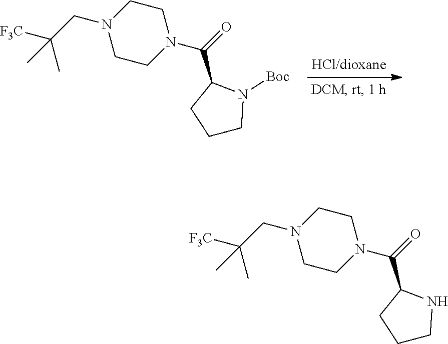 [SMILES: CC(C)(C)OC(=O)N1CCC[C@H]1C(=O)N1CCN(CC(C)(C)C(F)(F)F)CC1 ; CC(C)(CN1CCN(C(=O)[C@@H]2CCCN2)CC1)C(F)(F)F]

Tert-butyl (S)-2-(4-(3,3,3-trifluoro-2,2-dimethylpropyl)piperazin-1-carbonyl)pyrrolidin-1-carboxylate (0.300 g, 0.736 mmol) prepared in step 5 and hydrochloric acid (0.268 g, 7.362 mmol) were dissolved in dichloromethane (10 mL) at room temperature, after which the resulting solution was stirred at the same temperature for 18 hours. Solvent was removed from the reaction mixture under reduced pressure, after which an obtained product was used without an additional purification process (title compound, 0.200 g, 88.4%, brown oil).

### [Step 6] (S)-(1-(7-amino-2-(furan-2-yl)-[1,2,4]triazolo[1,5-a][1,3,5]triazin-5-yl)pyrrolidin-2-yl)(4-(3,3,3-trifluoro-2,2-dimethylpropyl)piperazin-1-yl)methanone

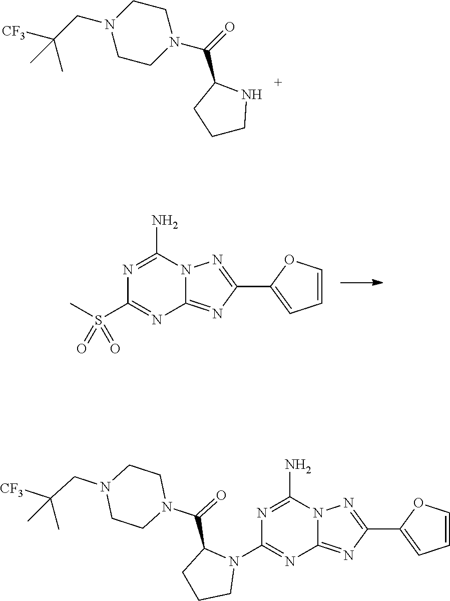 [SMILES: CC(C)(CN1CCN(C(=O)[C@@H]2CCCN2)CC1)C(F)(F)F ; CS(=O)(=O)c1nc(N)n2nc(-c3ccco3)nc2n1 ; CC(C)(CN1CCN(C(=O)[C@@H]2CCCN2c2nc(N)n3nc(-c4ccco4)nc3n2)CC1)C(F)(F)F]

(S)-1-Prolyl-4-(3,3,3-trifluoro-2,2-dimethylpropyl)piperazine (0.200 g, 0.651 mmol) prepared in step 5, 2-(furan-2-yl)-5-(methylsulfonyl)-[1,2,4]triazolo[1,5-a][1,3,5]triazin-7-amine (0.182 g, 0.651 mmol) and sodium hydrogen carbonate (0.164 g, 1.952 mmol) were dissolved in acetonitrile (5 mL) at room temperature, after which the resulting solution was stirred at 70° C. for 18 hours to complete the reaction by lowering a temperature to room temperature. Solvent was removed from the reaction mixture under reduced pressure, after which the resulting concentrate was purified via column chromatography (SiO₂, 4 g cartridge; ethyl acetate/hexane=0 to 100%) and concentrated to obtain a title compound (0.010 g, 3.0%) as a white solid form.

¹H NMR (400 MHz, DMSO-d₆) δ 8.30 (m, 2H), 7.87 (ddd, J=2.6, 1.8, 0.8 Hz, 1H), 7.06 (ddd, J=6.5, 3.4, 0.8 Hz, 1H), 6.69-6.61 (m, 1H), 5.06-4.90 (m, 1H), 3.76-3.40 (m, 6H), 2.77 (m, 1H), 2.60-2.34 (m, 5H), 2.26 (m, 1H), 1.96-1.75 (m, 3H), 1.11 (m, 6H); LRMS (ES) m/z 508.5 (M⁺+1).

### Examples 283,284,285, 286, 330 and 389

Example compounds 283, 284, 285, 286, 330 and 389 were each synthesized through substantially the same synthesis method as a synthesis method of example compound 282 except for using the compounds of the following table instead of 3,3,3-trifluoro-2,2-dimethylpropanoic acid as a starting material.

TABLE 43

| Example | Starting |
|---|---|
| No. | Materials |
| 283 | 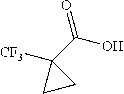 |
| 284 | 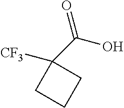 [SMILES: O=C(O)C1(C(F)(F)F)CCC1] |
| 285 | 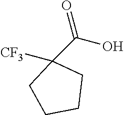 [SMILES: O=C(O)C1(C(F)(F)F)CCCC1] |
| 286 | 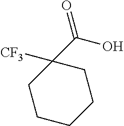 [SMILES: O=C(O)C1(C(F)(F)F)CCCCC1] |
| 330 | 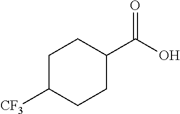 [SMILES: O=C(O)C1CCC(C(F)(F)F)CC1] |
| 389 | 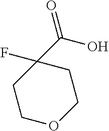 |

Analysis data of each of the compounds prepared as described above are shown in the table below.

TABLE 44

| Example | | |
|---|---|---|
| No. | Compound Names | Analysis Data |
| 283 | (S)-(1-(7-amino- | ¹H NMR (400 MHz, DMSO-d₆) |
| | 2-(furan-2-yl)- | δ 8.30 (m, 2H), 7.91- |
| | [1,2,4]triazolo[1,5- | 7.83 (m, 1H), 7.05 (ddd, |
| | a][1,3,5]triazin- | J = 9.1, 3.4, 0.7 Hz, 1H), |
| | 5-yl)pyrrolidin- | 6.71-6.61 (m, 1H), 5.00 |
| | 2-yl)(4-((1- | (,, 1H), 3.73-3.55 (m, 4H), |
| | (trifluorome- | 3.34 (m, 2H), 2.78-2.64 |
| | thyl)cyclopropyl) | (m, 2H), 2.63-2.11 (m, |
| | methyl)piperazin- | 5H), 1.88 (m, 3H), 1.02-0.70 |
| | 1-yl)methanone | (m, 4H); LRMS (ES) |
| | | m/z 506.5 (M⁺ + 1). |
| 284 | (S)-(1-(7-amino- | ¹H NMR (400 MHz, DMSO-d₆) |
| | 2-(furan-2-yl)- | δ 8.30 (m, 2H), 7.87 |
| | [1,2,4]triazolo[1,5- | (td, J = 2.1, 0.8 Hz, 1H), |
| | a][1,3,5]triazin- | 7.06 (ddd, J = 5.1, 3.4, 0.8 |
| | 5-yl)pyrrolidin- | Hz, 1H), 6.68 (dt, J = 3.4, |
| | 2-yl)(4-((1- | 1.7 Hz, 1H), 5.05-4.91 (m, |
| | (trifluorome- | 1H), 3.77-3.54 (m, 5H), |
| | thyl)cyclobutyl)met | 3-33 (m, 1H), 2.78-2.54 |
| | hyl)piperazin- | (m, 4H), 2.42-2.30 (m, 2H), |
| | 1-yl)methanone | 2.30-2.14 (m, 3H), |
| | | 2.08 (m, 2H), 2.00-1.73 |
| | | (m, 5H); LRMS (ES) m/z |
| | | 520.5 (M⁺ + 1). |
| 285 | (S)-(1-(7-amino- | ¹H NMR (400 MHz, DMSO-d₆) |
| | 2-(furan-2-yl)- | δ 8.30 (m, 2H), 7.87 |
| | [1,2,4]triazolo[1,5- | (ddd, J = 2.6, 1.7, 0.8 Hz, |
| | a][1,3,5]triazin- | 1H), 7.06 (ddd, J = 4.9, 3.4, |
| | 5-yl)pyrrolidin- | 0.7 Hz, 1H), 6.70-6.58 (m, |
| | 2-yl)(4-((1- | 1H), 5.04-4.89 (m, 1H), |
| | (trifluorome | 3.71-3.43 (m, 6H), 2.80-2.65 |
| | thyl)cyclohexyl)met | (m, 1H), 2.64-2.32 |
| | hyl)piperazin- | (m, 5H), 2.25 (m, 1H), 1.89 |
| | 1-yl)methanone | (m, 3H), 1.68 (m, 2H), |
| | | 1.58-1.37 (m, 6H), 1.23 (m, |
| | | 2H); LRMS (ES) m/z |
| | | 548.5 (M⁺ + 1). |
| 286 | (S)-(1-(7-amino-2- | ¹H NMR (400 MHz, DMSO-d₆) |
| | (furan-2-yl)- | δ 8.31 (m, 2H), 7.95- |
| | [1,2,4]triazolo[1,5- | 7.73 (m, 1H), 7.06 (ddd, |
| | a][1,3,5]triazin- | J = 5.8, 3.4, 0.7 Hz, 1H), |
| | 5-yl)pyrrolidin- | 6.68 (dt, J = 3.5, 1.9 Hz, |
| | 2-yl)(4-((1- | 1H), 5.05-4.89 (m, 1H), |
| | (trifluorome- | 3.72-3.42 (m, 5H), 3.33 (s, |
| | thyl)cyclopentyl)me | 1H), 2.73 (m, 1H), 2.63- |
| | thyl)piperazin- | 2.33 (m, 5H), 2.25 (m, 1H), |
| | 1-yl)methanone | 2.01-1.51 (m, 11H); |
| | | LRMS (ES) m/z 548.5 (M⁺ + 1). |
| 330 | (S)-(1-(7-amino- | ¹H NMR (400 MHz, DMSO-d₆) |
| | 2-(furan-2-yl)- | δ 8.29 (m, 2H), 7.87 |
| | [1,2,4]triazolo[1,5- | (d, J = 2.1 Hz, 1H), 7.06 (dd, |
| | a][1,3,5]triazin- | J = 5.9, 3.7 Hz, 1H), 6.68 |
| | 5-yl)pyrrolidin- | (dd, J = 4.9, 3.1 Hz, 1H), |
| | 2-yl)(4-((4- | 5.00 (m, 1H), 3.73-3.45 |
| | (trifluorome- | (m, 4H), 2.70-2.56 (m, 1H), |
| | thyl)cyclohexyl)met | 2.36-2.04 (m, 6H), |
| | hyl)piperazin- | 2.04-1.67 (m, 6H), 1.54 |
| | 1-yl)methanone | (m, 5H), 1.37-1.13 (m, |
| | | 4H); LRMS (ES) m/z 548.6 (M⁺ + 1). |
| 389 | (S)-(1-(7-amino- | ¹H NMR (400 MHz, DMSO-d₆) |
| | 2-(furan-2-yl)- | δ 8.33 (m, 2H), 7.88 |
| | [1,2,4]triazolo[1,5- | (s, 1H), 7.06 (t, J = 3.4 Hz, |
| | a][1,3,5]triazin- | 1H), 6.68 (dt, J = 3.6, 2.0 |
| | 5-yl)pyrrolidin- | Hz, 1H), 5.06-4.91 (m, 1H), |
| | 2-yl)(4-((4- | 3.76-3.48 (m, 8H), |
| | fluorotetrahydro-2H- | 3.32 (m, 2H), 2.82-2.55 |
| | pyran-4- | (m, 4H), 2.44-2.19 (m, |
| | yl)methyl)piperazin-1- | 2H), 1.97-1.61 (m, 8H); |
| | yl)methanone | LRMS (ES) m/z 500.39 |
| | | (M⁺ + 1). |

### Example 130: Synthesis of Compound 130, 1-((7-amino-2-(furan-2-yl)-[1,2,4]triazolo[1,5-a][1,3,5]triazin-5-yl)-L-prolyl)-N-(3-fluorophenyl)piperidin-4-carboxamide

### [Step 1] Synthesis of tert-butyl 4-((3-fluorophenyl)carbamoyl)piperidin-1-carboxylate

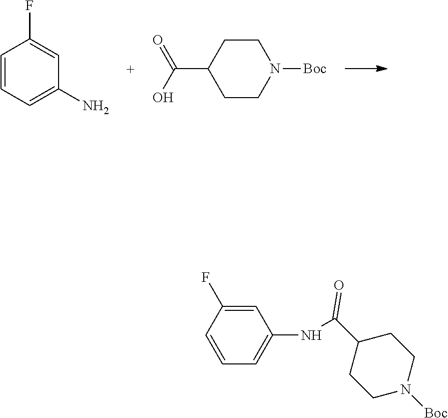 [SMILES: Nc1cccc(F)c1 ; CC(C)(C)OC(=O)N1CCC(C(=O)O)CC1 ; CC(C)(C)OC(=O)N1CCC(C(=O)Nc2cccc(F)c2)CC1]

1-(Tert-butoxycarbonyl)piperidin-4-carboxylic acid (0.229 g, 1.000 mmol), 3-fluoroaniline (0.096 mL, 1.000 mmol), 2,4,6-tripropyl-1,3,5,2,4,6-trioxatriphosphinane 2,4,6-trioxide (T3P, 50.00% solution in EtOAc, 0.892 mL, 1.500 mmol) and N,N-diisopropylethylamine (0.523 mL, 3.000 mmol) were dissolved in dichloromethane (5 mL) at room temperature, after which the resulting solution was stirred at the same temperature for 18 hours. Saturated aqueous solution of sodium hydrogen carbonate was poured into the reaction mixture, and then an organic layer was extracted with dichloromethane, filtered via a plastic filter to remove a solid residue and an aqueous solution layer therefrom, and concentrated under reduced pressure. An obtained product was used without an additional purification process (title compound, 0.322 g, 99.9%, light yellow oil).

### [Step 2] Synthesis of N-(3-fluorophenyl)piperidin-4-carboxamide hydrochloride

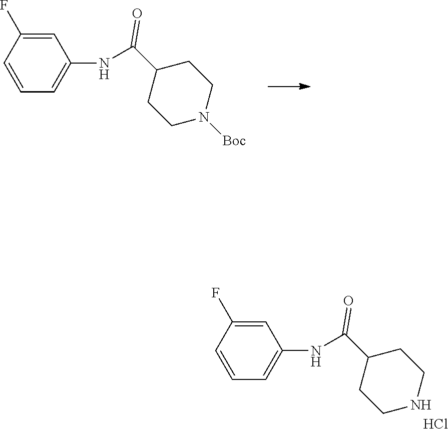 [SMILES: CC(C)(C)OC(=O)N1CCC(C(=O)Nc2cccc(F)c2)CC1 ; O=C(Nc1cccc(F)c1)C1CCNCC1 ; Cl]

Tert-butyl 4-((3-fluorophenyl)carbamoyl)piperidin-1-carboxylate (0.322 g, 0.999 mmol) prepared in step 1 and hydrogen chloride (4.00 M solution in 1,4-dioxane, 0.999 mL, 3.995 mmol) were dissolved in dichloromethane (4 mL) at room temperature, after which the resulting solution was stirred at the same temperature for 18 hours. Solvent was removed from the reaction mixture under reduced pressure, after which an obtained product was used without an additional purification process (title compound, 0.258 g, 99.8%, light yellow oil).

### [Step 3] Synthesis of tert-butyl (S)-2-(4-((3-fluorophenyl)carbamoyl)piperidin-1-carbonyl)pyrrolidin-1-carboxylate

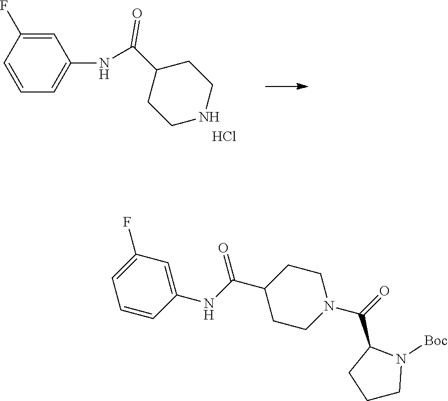 [SMILES: O=C(Nc1cccc(F)c1)C1CCNCC1 ; Cl ; CC(C)(C)OC(=O)N1CCC[C@H]1C(=O)N1CCC(C(=O)Nc2cccc(F)c2)CC1]

N-(3-fluorophenyl)piperidin-4-carboxamide hydrochloride (0.258 g, 0.997 mmol) prepared in step 2, (tert-butoxycarbonyl)-L-proline (0.215 g, 0.997 mmol), 2,4,6-tripropyl-1,3,5,2,4,6-trioxatriphosphinane 2,4,6-trioxide (T3P, 50.00% solution in EtOAc, 0.889 mL, 1.496 mmol) and N,N-diisopropylethylamine (0.521 mL, 2.992 mmol) were dissolved in dichloromethane (5 mL) at room temperature, after which the resulting solution was stirred at the same temperature for 18 hours. Saturated aqueous solution of sodium hydrogen carbonate was poured into the reaction mixture, and then an organic layer was extracted with dichloromethane, filtered via a plastic filter to remove a solid residue and an aqueous solution layer therefrom, and concentrated under reduced pressure. An obtained product was used without an additional purification process (title compound, 0.410 g, 98.0%, light yellow oil).

### [Step 4] Synthesis of (S)-N-(3-fluorophenyl)-1-prolylpiperidin-4-carboxamide hydrochloride

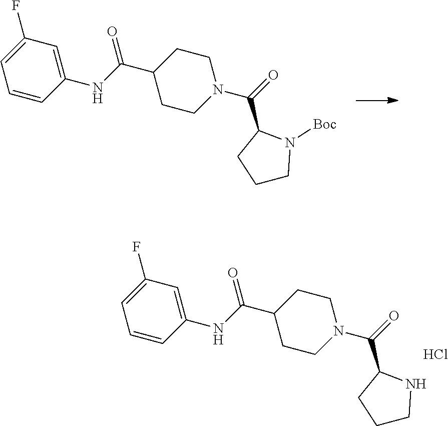 [SMILES: CC(C)(C)OC(=O)N1CCC[C@H]1C(=O)N1CCC(C(=O)Nc2cccc(F)c2)CC1 ; O=C(Nc1cccc(F)c1)C1CCN(C(=O)[C@@H]2CCCN2)CC1 ; Cl]

Tert-butyl (S)-2-(4-((3-fluorophenyl)carbamoyl)piperidin-1-carbonyl)pyrrolidin-1-carboxylate (0.419 g, 0.999 mmol) prepared in step 3 and hydrogen chloride (4.00 M solution in 1,4-dioxane, 0.999 mL, 3.995 mmol) were dissolved in dichloromethane (5 mL) at room temperature, after which the resulting solution was stirred at the same temperature for 18 hours. Solvent was removed from the reaction mixture under reduced pressure, after which an obtained product was used without an additional purification process (title compound, 0.350 g, 98.5%, light yellow oil).

### [Step 5] Synthesis of 1-((7-amino-2-(furan-2-yl)-[1,2,4]triazolo[1,5-a][1,3,5]triazin-5-yl)-L-prolyl)-N-(3-fluorophenyl)piperidin-4-carboxamide

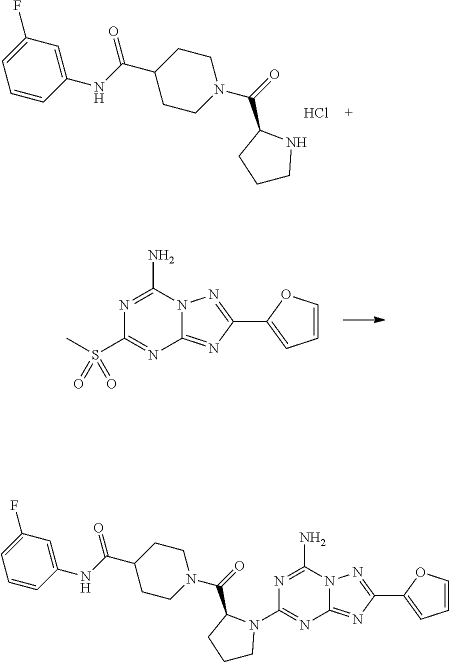 [SMILES: O=C(Nc1cccc(F)c1)C1CCN(C(=O)[C@@H]2CCCN2)CC1 ; Cl ; CS(=O)(=O)c1nc(N)n2nc(-c3ccco3)nc2n1 ; Nc1nc(N2CCC[C@H]2C(=O)N2CCC(C(=O)Nc3cccc(F)c3)CC2)nc2nc(-c3ccco3)nn12]

(S)-N-(3-fluorophenyl)-1-prolylpiperidin-4-carboxamide hydrochloride (0.356 g, 1.000 mmol) prepared in step 4, 2-(furan-2-yl)-5-(methylsulfonyl)-[1,2,4]triazolo[1,5-a][1,3,5]triazin-7-amine (0.280 g, 1.000 mmol) and sodium hydrogen carbonate (0.252 g, 3.001 mmol) were dissolved in cyclopentylmethyl ether (CPME, 5 mL) at room temperature, after which the resulting solution was stirred at 50° C. for 18 hours to complete the reaction by lowering a temperature to room temperature. Solvent was removed from the reaction mixture under reduced pressure, after which saturated aqueous solution of ammonium chloride was poured into the resulting concentrate, and then an organic layer was extracted with dichloromethane, filtered via a plastic filter to remove a solid residue and an aqueous solution layer therefrom, and concentrated under reduced pressure. The resulting concentrate was purified via column chromatography (SiO₂, 12 g cartridge; dichloromethane/methanol=0 to 5%) and concentrated to obtain a title compound (0.109 g, 21.0%) as a white solid form.

¹H NMR (400 MHz, DMSO-d₆) δ 10.46-10.07 (m, 1H), 8.27 (d, J=95.0 Hz, 1H), 7.89-7.81 (m, 1H), 7.67 (dd, J=40.6, 11.7 Hz, 1H), 7.33 (d, J=8.0 Hz, 2H), 7.05 (dd, J=9.5, 3.4 Hz, 1H), 6.95-6.83 (m, 1H), 6.68 (td, J=3.4, 1.8 Hz, 1H), 5.12-4.92 (m, 1H), 4.44-4.28 (m, 1H), 4.21-4.01 (m, 1H), 3.71-3.56 (m, 2H), 3.30-3.12 (m, 1H), 2.82-2.58 (m, 2H), 2.39-2.13 (m, 1H), 1.98-1.77 (m, 5H), 1.68-1.39 (m, 2H). LRMS (ES) m/z 521.5 (M⁺+1).

### Examples 131, 132 and 133

Example compounds 131, 132 and 133 were each synthesized through substantially the same synthesis method as a synthesis method of example compound 130 except for using the compounds of the following table instead of 3-fluoroaniline as a starting material.

TABLE 45

| Example | Starting |
|---|---|
| No. | Materials |
| 131 | 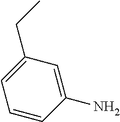 |
| 132 | 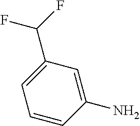 |
| 133 | 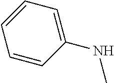 |

Analysis data of each of the compounds prepared as described above are shown in the table below.

TABLE 46

| Example | | |
|---|---|---|
| No. | Compound Names | Analysis Data |
| 131 | 1-((7-amino-2- | ¹H NMR (400 MHz, DMSO-d₆) |
| | (furan-2-yl)- | δ 10.21-9.82 (m, |
| | [1,2,4]triazolo[1,5- | 1H), 8.16 (s, 2H), 7.91-7.81 |
| | a][1,3,5]triazin- | (m, 1H), 7.61-7.38 |
| | 5-yl)-L-prolyl)-N-(3- | (m, 2H), 7.26-7.15 (m, 1H), |
| | ethylphenyl)piperidin-4- | 7.06 (dd, J = 10.0, |
| | carboxamide | 3.7 Hz, 1H), 6.94 (dd, |
| | | J = 16.4, 7.6 Hz, 1H), 5.16- |
| | | 4.91 (m, 1H), 4.46-4.26 |
| | | (m, 1H), 4.12 (dd, J = |
| | | 41.7, 14.3 Hz, 1H), 3.65 |
| | | (dt, J = 11.9, 6.9 Hz, 2H), |
| | | 2.93-2.56 (m, 3H), |
| | | 2.40-2.17 (m, 2H), 1.98- |
| | | 1.72 (m, 6H), 1.20 (dt, |
| | | J = 6.8, 5.0 Hz, 7H). LRMS |
| | | (ES) m/z 531.6 (M⁺ + 1). |
| 132 | 1-((7-amino-2- | ¹H NMR (400 MHz, |
| | (furan-2-yl)- | DMSO-d₆) δ 8.01-7.82 (m, |
| | [1,2,4]triazolo[1,5- | 2H), 7.70 (s, 1H), 7.46 |
| | a][1,3,5]triazin- | (q, J = 7.9 Hz, 1H), 7.30- |
| | 5-yl)-L-prolyl)-N-(3- | 7.13 (m, 1H), 7.11-7.00 |
| | (difluorome- | (m, 1H), 6.71-6.60 (m, |
| | thyl)phenyl)piperidin- | 1H), 5.03 (td, J = 25.4, |
| | 4-carboxamide | 24.9, 8.1 Hz, 1H), 4.44- |
| | | 4.26 (m, 1H), 4.22-4.01 |
| | | (m, 1H), 3.64 (s, 3H), |
| | | 2.86-2.57 (m, 3H), |
| | | 2.37-2.11 (m, 2H), 2.03- |
| | | 1.76 (m, 5H), 1.54 (d, J = 58.1 |
| | | Hz, 2H). LRMS (ES) |
| | | m/z 553.6 (M⁺ + 1). |
| 133 | 1-((7-amino-2- | ¹H NMR (400 MHz, |
| | (furan-2-yl)- | Chloroform-d) δ 8.75 (s, 1H), |
| | [1,2,4]triazolo[1,5- | 7.57 (dd, J = 1.7, 0.8 Hz, |
| | a][1,3,5]triazin- | 1H), 7.53-7.40 (m, 4H), |
| | 5-yl)-L-prolyl)- | 7.24-7.15 (m, 4H), 6.55 |
| | N-methyl-N- | (dd, J = 3.4, 1.8 Hz, 1H), |
| | phenylpiperidin-4- | 6.22 (s, 1H), 4.81 (dd, |
| | carboxamide | J = 8.7, 4.0 Hz, 1H), 4.56 |
| | | (d, J = 13.5 Hz, 1H), |
| | | 3.96-3.79 (m, 4H), 2.92 |
| | | (ddd, J = 14.8, 12.9, 2.4 |
| | | Hz, 1H), 2.67-2.57 (m, |
| | | 2H), 2.51 (qd, J = 12.5, |
| | | 4.0 Hz, 1H), 2.38-2.21 |
| | | (m, 3H), 2.21-2.09 (m, 1H), |
| | | 2.07-1.80 (m, 5H), |
| | | 1.69-1.54 (m, 3H), |
| | | 1.33-1.19 (m, 1H). LRMS |
| | | (ES) m/z 517.5 (M⁺ + 1). |

### Example 331: Synthesis of Compound 331, (2-(7-amino-2-(furan-2-yl)-[1,2,4]triazolo[1,5-a][1,3,5]triazin-5-yl)pyrazolidin-1-yl)(4-(2,2,2-trifluoroethyl)piperazin-1-yl)methanone

### [Step 1] Synthesis of tert-butyl 4-(4-(trifluoromethyl)piperidin-1-carbonyl)piperidin-1-carboxylate

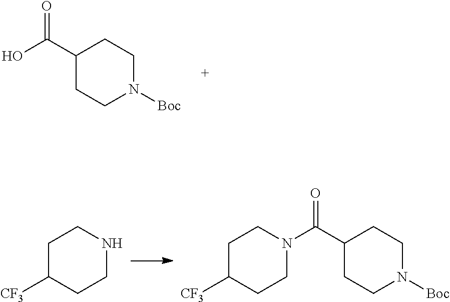 [SMILES: CC(C)(C)OC(=O)N1CCC(C(=O)O)CC1 ; FC(F)(F)C1CCNCC1 ; CC(C)(C)OC(=O)N1CCC(C(=O)N2CCC(C(F)(F)F)CC2)CC1]

1-(Tert-butoxycarbonyl)piperidin-4-carboxylic acid 3.000 g, 13.084 mmol), oxalyl dichloride (1.122 mL, 13.084 mmol) and N,N-dimethylformamide (0.050 mL, 0.654 mmol) were dissolved in dichloromethane (30 mL) at room temperature, after which the resulting solution was stirred at the same temperature for one hour. Here, 4-(trifluoromethyl)piperidine (1.236 g, 8.074 mmol) and triethylamine (1.688 mL, 12.110 mmol) were added into the resulting solution dissolved in dichloromethane (5 mL) at room temperature and stirred at the same temperature for 18 hours. Solvent was removed from the reaction mixture under reduced pressure, after which water was poured into the resulting concentrate and an organic layer was extracted with ethyl acetate. The organic layer was washed with saturated aqueous solution of sodium chloride, dehydrated with anhydrous magnesium sulfate, filtered, and concentrated under reduced pressure. An obtained product was used without an additional purification process (title compound, 1.500 g, 102.0%, white solid).

### [Step 2] Synthesis of tert-butyl 4-((4-(trifluoromethyl)piperidin-1-yl)methyl)piperidin-1-carboxylate

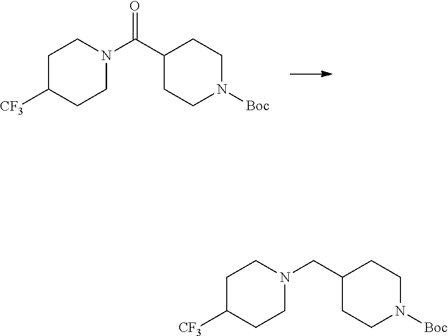 [SMILES: CC(C)(C)OC(=O)N1CCC(C(=O)N2CCC(C(F)(F)F)CC2)CC1 ; CC(C)(C)OC(=O)N1CCC(CN2CCC(C(F)(F)F)CC2)CC1]

Tert-butyl 4-(4-(trifluoromethyl)piperidin-1-carbonyl)piperidin-1-carboxylate (1.500 g, 4.116 mmol) prepared in step 1, trifluoroborane (45.00% solution in Et₂O, 0.457 mL, 20.581 mmol) and sodium borohydride (0.311 g, 8.232 mmol) were dissolved in tetrahydrofuran (20 mL) at room temperature, after which the resulting solution was stirred at the same temperature for 18 hours. Solvent was removed from the reaction mixture under reduced pressure, after which saturated aqueous solution of sodium hydrogen carbonate was poured into the resulting concentrate, and an organic layer was extracted with ethyl acetate. The organic layer was washed with saturated aqueous solution of sodium chloride, dehydrated with anhydrous magnesium sulfate, filtered, and concentrated under reduced pressure. An obtained product was used without an additional purification process (title compound, 0.500 g, 34.7%, white solid).

### [Step 3] Synthesis of 1-(piperidin-4-ylmethyl)-4-(trifluoromethyl)piperidine

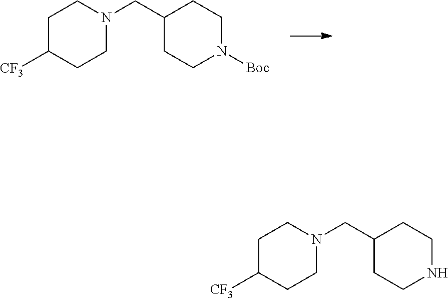 [SMILES: CC(C)(C)OC(=O)N1CCC(CN2CCC(C(F)(F)F)CC2)CC1 ; FC(F)(F)C1CCN(CC2CCNCC2)CC1]

Tert-butyl 4-((4-(trifluoromethyl)piperidin-1-yl)methyl)piperidin-1-carboxylate (0.500 g, 1.427 mmol) prepared in step 2 and hydrochloric acid (4.00 M solution in dioxane, 1.784 mL, 7.134 mmol) were dissolved in dichloromethane (10 mL) at room temperature, after which the resulting solution was stirred at the same temperature for 18 hours. Solvent was removed from the reaction mixture under reduced pressure, after which an obtained product was used without an additional purification process (title compound, 0.300 g, 84.0%, white solid).

### [Step 4] Synthesis of tert-butyl (S)-2-(4-((4-(trifluoromethyl)piperidin-1-yl)methyl)piperidin-1-carbonyl)pyrrolidine-1-carboxylate

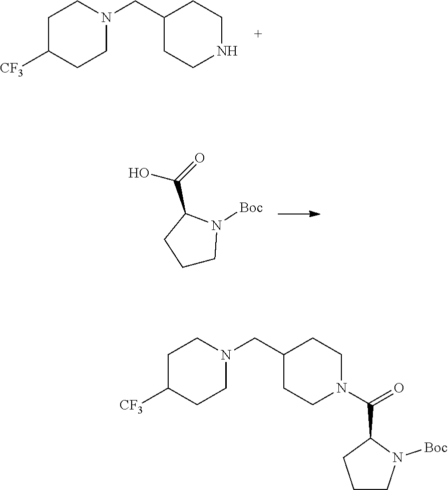 [SMILES: FC(F)(F)C1CCN(CC2CCNCC2)CC1 ; CC(C)(C)OC(=O)N1CCC[C@H]1C(=O)O ; CC(C)(C)OC(=O)N1CCC[C@H]1C(=O)N1CCC(CN2CCC(C(F)(F)F)CC2)CC1]

1-(Piperidin-4-ylmethyl)-4-(trifluoromethyl)piperidine (0.300 g, 1.199 mmol) prepared in step 3, (tert-butoxycarbonyl)-L-proline (0.516 g, 2.397 mmol), 1-[bis(dimethylamino)methylene]-1H-1,2,3-triazolo[4,5-b]pyridinium 3-oxide hexafluorophosphate (HATU, 0.911 g, 2.397 mmol) and N,N-diisopropylethylamine (1.044 mL, 5.993 mmol) were dissolved in dichloromethane (30 mL) at room temperature, after which the resulting solution was stirred at the same temperature for 18 hours. Solvent was removed from the reaction mixture under reduced pressure, after which aqueous solution of N-sodium hydrogen carbonate was poured into the resulting concentrate, and an organic layer was extracted with ethyl acetate. The organic layer was washed with saturated aqueous solution of sodium chloride, dehydrated with anhydrous magnesium sulfate, filtered, and concentrated under reduced pressure. An obtained product was used without an additional purification process (title compound, 0.500 g, 93.2%, brown oil).

### [Step 5] Synthesis of (S)-1-prolyl-4-((4-(trifluoromethyl)piperidin-1-yl)methyl)piperidine

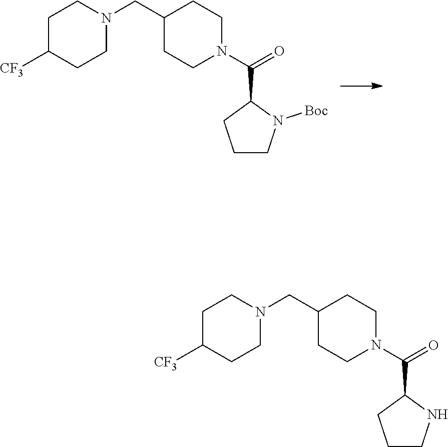 [SMILES: CC(C)(C)OC(=O)N1CCC[C@H]1C(=O)N1CCC(CN2CCC(C(F)(F)F)CC2)CC1 ; O=C([C@@H]1CCCN1)N1CCC(CN2CCC(C(F)(F)F)CC2)CC1]

Tert-butyl (S)-2-(4-((4-(trifluoromethyl)piperidin-1-yl)methyl)piperidin-1-carbonyl)pyrrolidin-1-carboxylate (0.300 g, 0.670 mmol) prepared in step 4 and hydrochloric acid (4.00 M solution in dioxane, 1.676 mL, 6.703 mmol) were dissolved in dichloromethane (20 mL) at room temperature, after which the resulting solution was stirred at the same temperature for 18 hours. Solvent was removed from the reaction mixture under reduced pressure, after which an obtained product was used without an additional purification process (title compound, 0.200 g, 85.9%, brown oil).

### [Step 6] Synthesis of (S)-(1-(7-amino-2-(furan-2-yl)-[1,2,4]triazolo[1,5-a][1,3,5]triazin-5-yl)pyrrolidin-2-yl)(4-((4-(trifluoromethyl)piperidin-1-yl)methyl)piperidin-1-yl)methanone

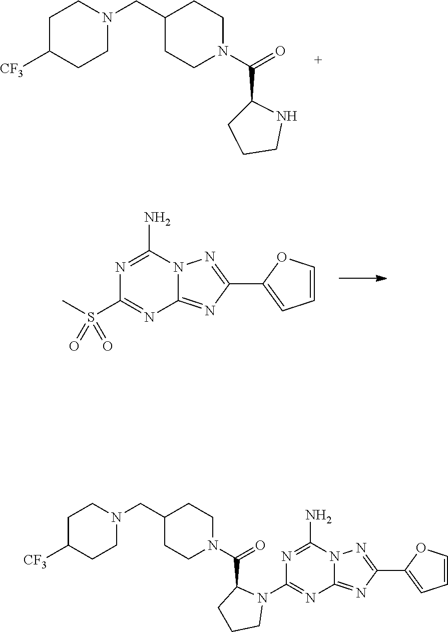 [SMILES: O=C([C@@H]1CCCN1)N1CCC(CN2CCC(C(F)(F)F)CC2)CC1 ; CS(=O)(=O)c1nc(N)n2nc(-c3ccco3)nc2n1 ; Nc1nc(N2CCC[C@H]2C(=O)N2CCC(CN3CCC(C(F)(F)F)CC3)CC2)nc2nc(-c3ccco3)nn12]

(S)-1-Prolyl-4-((4-(trifluoromethyl)piperidin-1-yl)methyl)piperidine (0.300 g, 0.863 mmol) prepared in step 5, 2-(furan-2-yl)-5-(methylsulfonyl)-[1,2,4]triazolo[1,5-a][1,3,5]triazin-7-amine (0.060 g, 0.216 mmol) and sodium hydrogen carbonate (0.218 g, 2.590 mmol) were dissolved in acetonitrile (20 mL) at room temperature, after which the resulting solution was stirred at 70° C. for 18 hours to complete the reaction by lowering a temperature to room temperature. Solvent was removed from the reaction mixture under reduced pressure, after which the resulting concentrate was purified via chromatography (SiO₂ plate, 20×20×1 mm; methanol/dichloromethane=10%) and concentrated to obtain a title compound (0.005 g, 1.1%) as a white solid form.

¹H NMR (400 MHz, DMSO-d₆) δ 8.56-8.03 (m, 2H), 7.87 (d, J=1.7 Hz, 1H), 7.09-6.95 (m, 1H), 6.68 (s, 1H), 5.01 (m, 1H), 4.41-3.87 (m, 2H), 3.73-3.44 (m, 4H), 3.24-2.85 (m, 6H), 2.38-2.02 (m, 4H), 2.01-1.57 (m, 8H), 1.48 (s, 1H), 1.10-0.75 (m, 2H); LRMS (ES) m/z 548.6 (M⁺+1).

### Example 402: Synthesis of Compound 402

Example compound 402 was synthesized through substantially the same synthesis method as a synthesis method of example compound 331 except for using (S)-1-(tert-butoxycarbonyl)azetidin-2-carboxylic acid instead of (tert-butoxycarbonyl)-L-proline.

### Example 406: Synthesis of Compound 406

Example compound 406 was prepared through substantially the same synthesis method as a synthesis method of example compound 331 except for using 3-fluoroazetidine instead of 4-(trifluoromethyl)piperidine.

Analysis data of each of the compounds prepared as described above are shown in the table below.

TABLE 47

| Example | | |
|---|---|---|
| No. | Compound Names | Analysis Data |
| 402 | (S)-(1-(7-amino- | ¹H NMR (400 MHz, |
| | 2-(furan-2-yl)- | DMSO-d₆) δ 8.68-8.08 (m, |
| | [1,2,4]triazolo[1,5- | 2H), 7.87 (d, J = 2.1 Hz, |
| | a][1,3,5]triazin-5- | 1H), 7.04 (d, J = 8.1 Hz, |
| | yl)azetidin-2-yl)(4-((4- | 1H), 6.67 (dt, J = 3.2, 1.5 |
| | (trifluorome- | Hz, 1H), 5.23 (q, J = 9.4, |
| | thyl)piperidin-1- | 7.0 Hz, 1H), 4.35 (d, |
| | yl)methyl)piperidin- | J = 12.9 Hz, 1H), 4.09-3.43 |
| | 1-yl)methanone | (m, 9H), 3.13-2.87 (m, 5H), |
| | | 2.66 (d, J = 14.4 Hz, |
| | | 3H), 2.19-1.93 (m, 4H), |
| | | 1.92-1.73 (m, 4H). |
| | | LRMS (ES) m/z 533.88 (M⁺ + 1). |
| 406 | (S)-(1-(7-amino- | ¹H NMR (400 MHz, DMSO-d₆) |
| | 2-(furan-2-yl)- | δ 8.32 (d, J = 121.3 |
| | [1,2,4]triazolo[1,5- | Hz, 2H), 7.88 (d, J = 4.0 |
| | a][1,3,5]triazin-5- | Hz, 1H), 7.14-6.96 (m, |
| | yl)pyrrolidin-2-yl)(4-((3- | 1H), 6.69 (d, J = 3.2 Hz, |
| | fluoroazetidin-1- | 1H), 5.28-4.94 (m, 2H), |
| | yl)methyl)piperidin- | 4.39-3.91 (m, 3H), 3.61 |
| | 1-yl)methanone | (qd, J = 14.7, 13.6, 6.9 |
| | | Hz, 4H), 3.19-2.97 (m, 3H), |
| | | 2.37-2.19 (m, 2H), |
| | | 2.01-1.48 (m, 8H), 1.07 |
| | | (dd, J = 17.9, 8.9 Hz, 1H). |
| | | LRMS (ES) m/z 470.34 (M⁺ + 1). |

### Example 186: Synthesis of Compound 186, (S)-2-((7-amino-2-(furan-2-yl)-[1,2,4]triazolo[1,5-a][1,3,5]triazin-5-yl)amino)-2-cyclohexyl-1-(4-(3,3-difluoroazetidin-1-yl)piperidin-1-yl)ethan-1-one

### [Step 1] Synthesis of tert-butyl 4-(3,3-difluoroazetidin-1-yl)piperidin-1-carboxylate

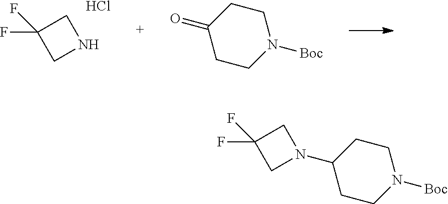 [SMILES: FC1(F)CNC1 ; Cl ; CC(C)(C)OC(=O)N1CCC(=O)CC1 ; CC(C)(C)OC(=O)N1CCC(N2CC(F)(F)C2)CC1]

Tert-butyl 4-oxopiperidin-1-carboxylate (0.500 g, 2.509 mmol), 3,3-difluoroazetidine (0.325 g, 2.509 mmol), sodium triacetoxyborohydride (0.798 g, 3.764 mmol) and triethylamine (0.350 mL, 2.509 mmol) were dissolved in dichloromethane (10 mL) at room temperature, after which the resulting solution was stirred at the same temperature for 18 hours. Aqueous solution of N-sodium hydrogen carbonate was poured into the reaction mixture, and then an organic layer was extracted with dichloromethane, filtered via a plastic filter to remove a solid residue and an aqueous solution layer therefrom, and concentrated under reduced pressure. An obtained product was used without an additional purification process (title compound, 0.653 g, 94.2%, transparent oil).

### [Step 2] Synthesis of 4-(3,3-difluoroazetidin-1-yl)piperidine hydrochloride

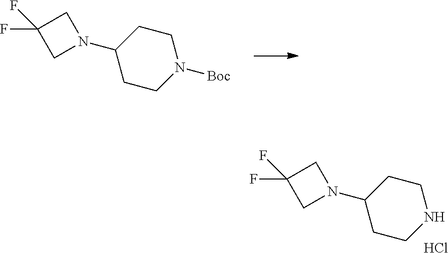 [SMILES: CC(C)(C)OC(=O)N1CCC(N2CC(F)(F)C2)CC1 ; FC1(F)CN(C2CCNCC2)C1 ; Cl]

Tert-butyl 4-(3,3-difluoroazetidin-1-yl)piperidin-1-carboxylate (0.653 g, 2.363 mmol) prepared in step 1 and hydrogen chloride (4.00 M solution in 1,4-dioxane, 2.363 mL, 9.452 mmol) were dissolved in dichloromethane (5 mL) at room temperature, after which the resulting solution was stirred at the same temperature for 18 hours. Solvent was removed from the reaction mixture under reduced pressure, after which an obtained product was used without an additional purification process (title compound, 0.502 g, 99.9%, white solid).

### [Step 3] Synthesis of tert-butyl (S)-(1-cyclohexyl-2-(4-(3,3-difluoroazetidin-1-yl)piperidin-1-yl)-2-oxoethyl)carbamate

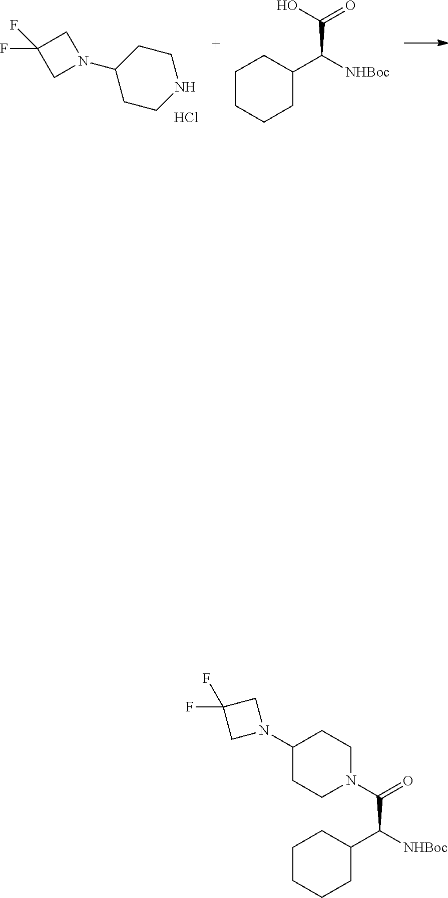 [SMILES: FC1(F)CN(C2CCNCC2)C1 ; Cl ; CC(C)(C)OC(=O)N[C@H](C(=O)O)C1CCCCC1 ; CC(C)(C)OC(=O)N[C@H](C(=O)N1CCC(N2CC(F)(F)C2)CC1)C1CCCCC1]

4-(3,3-Difluoroazetidin-1-yl)piperidine hydrochloride (0.258 g, 1.213 mmol) prepared in step 2, (S)-2-((tert-butoxycarbonyl)amino)-2-cyclohexylacetic acid (0.312 g, 1.213 mmol), 2,4,6-tripropyl-1,3,5,2,4,6-trioxatriphosphinane 2,4,6-trioxide (T3P, 50.00% solution in EtOAc, 1.082 mL, 1.820 mmol) and N,N-diisopropylethylamine (0.634 mL, 3.639 mmol) were dissolved in dichloromethane (5 mL) at room temperature, after which the resulting solution was stirred at the same temperature for 18 hours. Aqueous solution of N-sodium hydrogen carbonate was poured into the reaction mixture, and then an organic layer was extracted with dichloromethane, filtered via a plastic filter to remove a solid residue and an aqueous solution layer therefrom, and concentrated under reduced pressure. An obtained product was used without an additional purification process (title compound, 0.415 g, 82.3%, transparent oil).

### [Step 4] Synthesis of (S)-2-amino-2-cyclohexyl-1-(4-(3,3-difluoroazetidin-1-yl)piperidin-1-yl)ethan-1-one hydrochloride

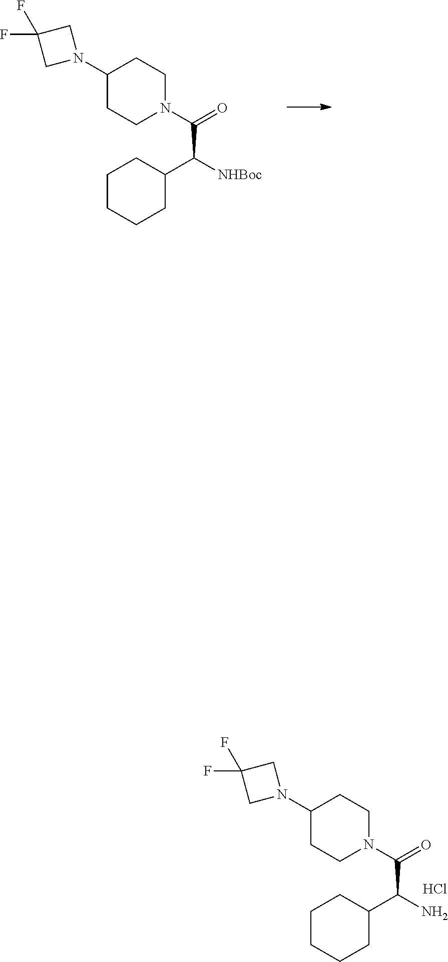 [SMILES: CC(C)(C)OC(=O)N[C@H](C(=O)N1CCC(N2CC(F)(F)C2)CC1)C1CCCCC1 ; N[C@H](C(=O)N1CCC(N2CC(F)(F)C2)CC1)C1CCCCC1 ; Cl]

Tert-butyl (S)-(1-cyclohexyl-2-(4-(3,3-difluoroazetidin-1-yl)piperidin-1-yl)-2-oxoethyl)carbamate (0.415 g, 0.999 mmol) prepared in step 3 and hydrogen chloride (4.00 M solution in 1,4-dioxane, 0.999 mL, 3.995 mmol) were dissolved in dichloromethane (5 mL) at room temperature, after which the resulting solution was stirred at the same temperature for 18 hours. Solvent was removed from the reaction mixture under reduced pressure, after which an obtained product was used without an additional purification process (title compound, 0.351 g, 99.9%, light yellow solid).

### [Step 5] Synthesis of (S)-2-((7-amino-2-(furan-2-yl)-[1,2,4]triazolo[1,5-a][1,3,5]triazin-5-yl)amino)-2-cyclohexyl-1-(4-(3,3-difluoroazetidin-1-yl)piperidin-1-yl)ethan-1-one

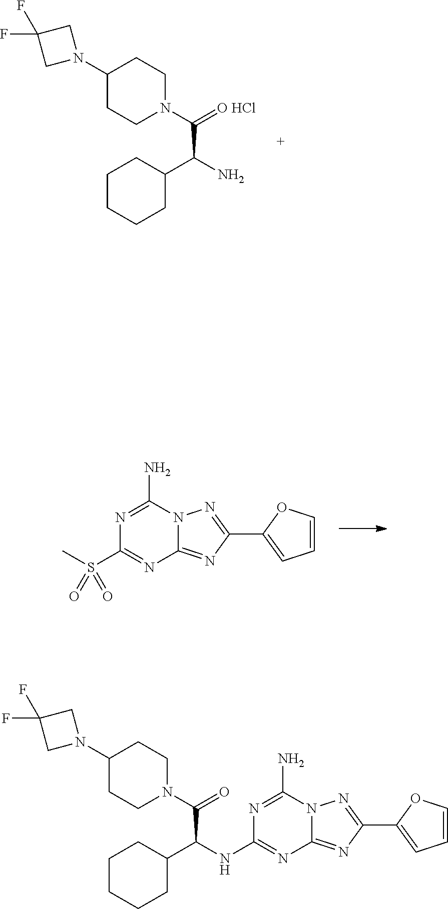

(S)-2-Amino-2-cyclohexyl-1-(4-(3,3-difluoroazetidin-1-yl)piperidin-1-yl)ethan-1-one hydrochloride (0.351 g, 0.998 mmol) prepared in step 4, 2-(furan-2-yl)-5-(methylsulfonyl)-[1,2,4]triazolo[1,5-a][1,3,5]triazin-7-amine (0.280 g, 0.998 mmol) and sodium hydrogen carbonate (0.251 g, 2.993 mmol) were dissolved in acetonitrile (5 mL) at room temperature, after which the resulting solution was stirred at 70° C. for 18 hours to complete the reaction by lowering a temperature to room temperature. The reaction mixture was filtered via a plastic filter to remove a solid therefrom, after which solvent was removed from the resulting filtrate without the solid under reduced pressure, and then the resulting concentrate was purified via column chromatography (SiO₂, 12 g cartridge; dichloromethane/methanol=0 to 10%) and concentrated to obtain a product, after which the obtained product was purified again via chromatography (SiO₂ plate, 20×20×1 mm; dichloromethane/methanol) and concentrated to obtain a title compound (0.041 g, 7.9%) as a white solid form.

¹H NMR (400 MHz, Chloroform-d) δ 9.71 (s, 1H), 8.47 (dd, J=23.4, 9.4 Hz, 1H), 7.57 (dd, J=3.1, 1.7 Hz, 1H), 7.23-7.07 (m, 1H), 6.54 (dt, J=3.8, 2.0 Hz, 1H), 6.28 (s, 1H), 5.23-5.05 (m, 1H), 4.41-4.05 (m, 2H), 3.66-3.44 (m, 5H), 3.22-3.09 (m, 1H), 2.51-2.35 (m, 1H), 1.96-1.52 (m, 9H), 1.21-0.89 (m, 6H). LRMS (ES) m/z 516.6 (M⁺+1).

### Example 390: Synthesis of Compound 390

Example compound 390 was synthesized through substantially the same synthesis method as a synthesis method of example compound 186 except for using (tert-butoxycarbonyl)-L-proline instead of (S)-2-((tert-butoxycarbonyl)amino)-2-cyclohexylacetic acid.

### Example 391: Synthesis of Compound 391

Example compound 391 was prepared through substantially the same synthesis method as a synthesis method of example compound 390 except for using 4,4-difluoropiperidine instead of 3,3-difluoroazetidine.

### Example 400: Synthesis of Compound 400

Example compound 400 was synthesized through substantially the same synthesis method as a synthesis method of example compound 390 except for using 2-(furan-2-yl)-7-(methylsulfonyl)-[1,2,4]triazolo[1,5-c]pyrimidin-5-amine instead of 2-(furan-2-yl)-5-(methylsulfonyl)-[1,2,4]triazolo[1,5-a][1,3,5]triazin-7-amine.

### Example 405: Synthesis of Compound 405

Example compound 405 was synthesized through substantially the same synthesis method as a synthesis method of example compound 390 except for using 1-(2-methoxyethyl)piperazine instead of 3,3-difluoroazetidine and using tert-butyl 3-oxoazetidin-1-carboxylate instead of tert-butyl 4-oxopiperidin-1-carboxylate.

Analysis data of each of the compounds prepared as described above are shown in the table below.

TABLE 48

| Example | | |
|---|---|---|
| No. | Compound Names | Analysis Data |
| 390 | (S)-(1-(7-amino- | ¹H NMR (400 MHz, DMSO-d₆) |
| | 2-(furan-2- | δ 8.68-8.00 (m, 2H), |
| | yl)-[1,2,4]triazolo[1,5- | 7.92-7.81 (m, 1H), 7.10-6.99 |
| | a][1,3,5]triazin-5- | (m, 1H), 6.74-6.62 |
| | yl)pyrrolidin- | (m, 1H), 5.07-4.89 (m, 1H), |
| | 2-yl)(4-(3,3- | 4.22-3.78 (m, 3H), 3.61 |
| | difluoroazetidin-1- | (q, J = 12.8 Hz, 5H), |
| | yl)piperidin- | 3.30-2.62 (m, 3H), 2.40-2.17 |
| | 1-yl)methanone | (m, 2H), 2.02-1.76 (m, 4H), |
| | | 1.75-1.44 (m, 1H), 1.34- |
| | | 0.99 (m, 1H); LRMS (ES) |
| | | m/z 474.3 (M⁺ + 1). |
| 391 | (S)-(1-(7-amino- | ¹H NMR (400 MHz, DMSO-d₆) |
| | 2-(furan-2- | δ 8.66-7.98 (m, 2H), |
| | yl)-[1,2,4]triazolo[1,5- | 7.91-7.78 (m, 1H), 7.11-6.92 |
| | a][1,3,5]triazin-5- | (m, 1H), 6.74-6.61 (m, |
| | yl)pyrrolidin-2-yl)(4,4- | 1H), 5.09-4.88 (m, 1H), |
| | difluoro- | 4.47-4.23 (m, 1H), 4.23- |
| | [1,4′-bipiperidin]-1′- | 3.96 (m, 1H), 3.74-3.50 (m, |
| | yl) methanone | 2H), 3.27-2.97 (m, 1H), |
| | | 2.87-2.55 (m, 5H), 2.37-2.18 |
| | | (m, 1H), 2.13-1.59 |
| | | (m, 10H), 1.46-1.15 (m, 2H); |
| | | LRMS (ES) m/z 502.3 |
| | | (M⁺ + 1). |
| 400 | (S)-(1-(5-amino- | ¹H NMR (400 MHz, DMSO-d₆) |
| | 2-(furan-2- | δ 7.88-7.82 (m, 1H), |
| | yl)-[1,2,4]triazolo[1,5- | 7.64-7.30 (m, 2H), 7.10-7.00 |
| | c]pyrimidin- | (m, 1H), 6.67 (dd, J = |
| | 7-yl)pyrrolidine- | 3.3, 1.8 Hz, 1H), 5.62 (brs, |
| | 2-yl)(4-(3,3- | 1H), 4.94 (brs, 1H), 4.03- |
| | difluoroazetidin- | 3.82 (m, 2H), 3.71-3.54 |
| | 1-yl)piperidin-1- | (m, 4H), 3.52-3.36 (m, 2H), |
| | yl)methanone | 3.23-3.07 (m, 1H), 3.02-2.79 |
| | | (m, 1H), 2.33-2.16 |
| | | (m, 1H), 2.05-1.89 (m, 2H), |
| | | 1.89-1.76 (m, 2H), 1.75- |
| | | 1.57 (m, 2H), 1.29-1.07 |
| | | (m, 2H); LRMS (ES) m/z |
| | | 473.32 (M⁺ + 1). |
| 405 | (S)-(1-(7-amino- | ¹H NMR (400 MHz, DMSO-d₆) |
| | 2-(furan-2- | δ 8.81-8.09 (m, 2H), |
| | yl)-[1,2,4]triazolo[1,5- | 7.88 (d, J = 3.8 Hz, 1H), 7.08 |
| | a][1,3,5]triazin-5- | (dd, J = 6.7, 3.4 Hz, 1H), |
| | yl)pyrrolidin- | 6.69 (dt, J = 5.1, 2.6 Hz, 1H), |
| | 2-yl)(3-(4-(2- | 4.63-4.14 (m, 3H), 4.06- |
| | methoxy- | 3.71 (m, 2H), 3.71-3.55 |
| | ethyl)piperazin-1- | (m, 2H), 3.53-3.38 (m, |
| | yl)azetidin-1- | 3H), 3.24 (d, J = 7.0 Hz, 4H), |
| | yl)methanone | 2.69-2.55 (m, 2H), 2.48- |
| | | 2.26 (m, 6H), 2.25-1.95 |
| | | (m, 3H), 1.95-1.77 (m, 2H); |
| | | LRMS (ES) m/z 495.25 (M⁺ + 1). |

### Example 25: Synthesis of Compound 25, (S)-2-((7-amino-2-(furan-2-yl)-[1,2,4]triazolo[1,5-a][1,3,5]triazin-5-yl)amino)-1-(4-(2,2,2-trifluoroethyl)piperazin-1-yl)propan-1-one

### [Step 1] Synthesis of benzyl 4-((tert-butoxycarbonyl)-L-alanyl)piperazine-1-carboxylate

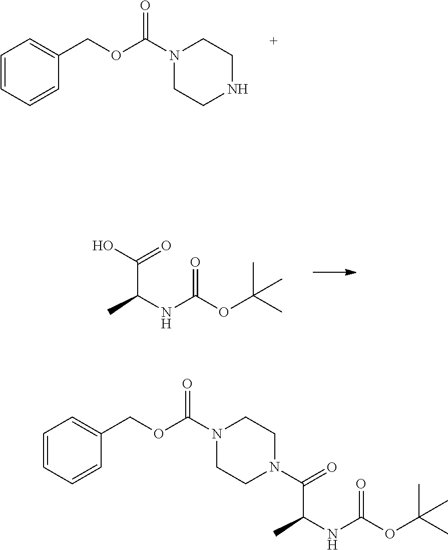 [SMILES: O=C(OCc1ccccc1)N1CCNCC1 ; C[C@H](NC(=O)OC(C)(C)C)C(=O)O ; C[C@H](NC(=O)OC(C)(C)C)C(=O)N1CCN(C(=O)OCc2ccccc2)CC1]

Benzyl piperazin-1-carboxylate (1.000 g, 4.540 mmol), (tert-butoxycarbonyl)-L-alanine (0.859 g, 4.540 mmol), 1-ethyl-3-(3-dimethylaminopropyl)carbodiimide hydrochloride (EDC-HCl, 2.611 g, 13.620 mmol), 1H-benzo[d][1,2,3]triazol-1-ol (HOBt, 1.840 g, 13.620 mmol) and N,N-diisopropylethylamine (3.954 mL, 22.699 mmol) were dissolved in dichloromethane (30 mL) at room temperature, after which the resulting solution was stirred at the same temperature for 18 hours. Water was poured into the reaction mixture and an organic layer was extracted with dichloromethane. The organic layer was washed with saturated aqueous solution of sodium chloride, dehydrated with anhydrous sodium sulfate, filtered, and concentrated under reduced pressure. The resulting concentrate was purified via column chromatography (SiO₂, 40 g cartridge; ethyl acetate/hexane=0 to 10%) and concentrated to obtain a title compound (1.200 g, 67.5%) as a colorless oil form.

### [Step 2] Synthesis of tert-butyl (S)-(1-oxo-1-(piperazin-1-yl)propan-2-yl)carbamate

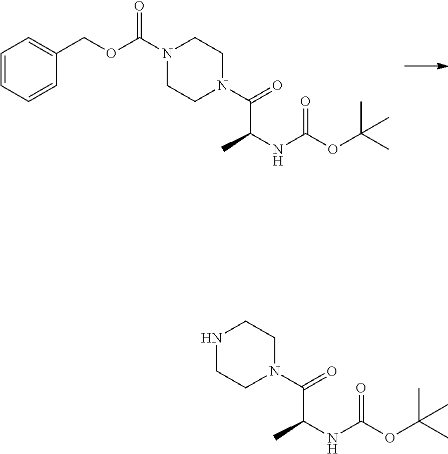 [SMILES: C[C@H](NC(=O)OC(C)(C)C)C(=O)N1CCN(C(=O)OCc2ccccc2)CC1 ; C[C@H](NC(=O)OC(C)(C)C)C(=O)N1CCNCC1]

Benzyl 4-((tert-butoxycarbonyl)-L-alanyl)piperazin-1-carboxylate (1.200 g, 3.065 mmol) prepared in step 1 was dissolved in methanol (20 mL) and stirred at room temperature, after which Pd/C (0.100 mg) was slowly added into the resulting solution at the same temperature and stirred for 18 hours in the presence of a hydrogen balloon attached thereto at the same temperature. The reaction mixture was filtered via a celite pad to remove a solid therefrom, after which solvent was removed from the resulting filtrate under reduced pressure, and then a product obtained was used without an additional purification process (title compound 0.746 g, 94.6%, colorless oil).

### [Step 3] Synthesis of tert-butyl (S)-(1-oxo-1-(4-(2,2,2-trifluoroethyl)piperazin-1-yl)propan-2-yl)carbamate

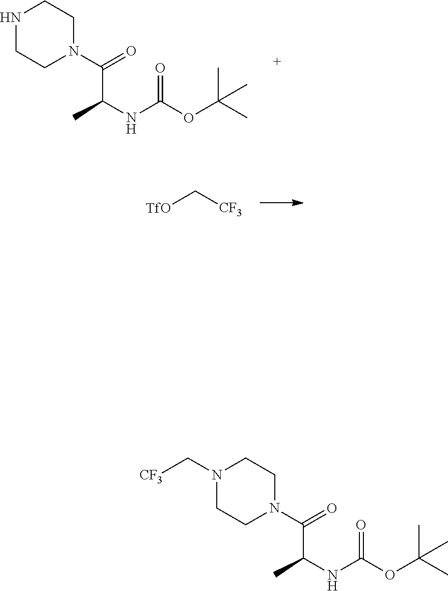 [SMILES: C[C@H](NC(=O)OC(C)(C)C)C(=O)N1CCNCC1 ; O=S(=O)(OCC(F)(F)F)C(F)(F)F ; C[C@H](NC(=O)OC(C)(C)C)C(=O)N1CCN(CC(F)(F)F)CC1]

Tert-butyl (S)-(1-oxo-1-(piperazin-1-yl)propan-2-yl)carbamate (0.080 g, 0.311 mmol) prepared in step 2, 2,2,2-trifluoroethyl trifluoromethanesulfonate (0.072 g, 0.311 mmol) and potassium carbonate (0.086 g, 0.622 mmol) were dissolved in acetonitrile (2 mL) at room temperature, after which the resulting solution was stirred at the same temperature for 18 hours. The reaction mixture was filtered via a plastic filter to remove a solid therefrom, after which solvent was removed from the resulting filtrate under reduced pressure, and then an obtained product was used without an additional purification process (title compound, 0.065 g, 61.6%, light brown oil).

### [Step 4] Synthesis of (S)-2-amino-1-(4-(2,2,2-trifluoroethyl)piperazin-1-yl)propan-1-one

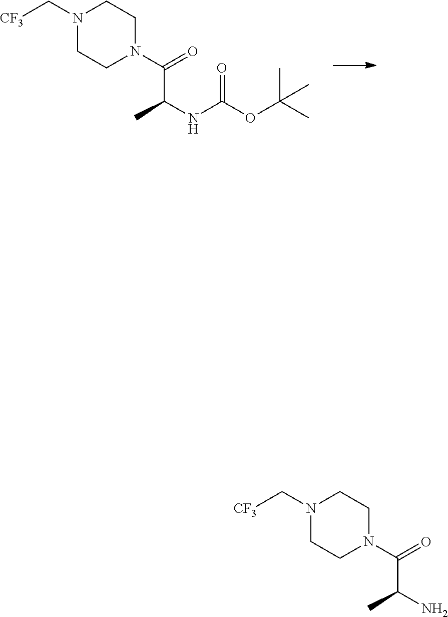 [SMILES: C[C@H](NC(=O)OC(C)(C)C)C(=O)N1CCN(CC(F)(F)F)CC1 ; C[C@H](N)C(=O)N1CCN(CC(F)(F)F)CC1]

Tert-butyl (S)-(1-oxo-1-(4-(2,2,2-trifluoroethyl)piperazin-1-yl)propan-2-yl)carbamate (0.065 g, 0.192 mmol) prepared in step 3 and trifluoroacetic acid (0.147 mL, 1.915 mmol) were dissolved in dichloromethane (5 mL) at room temperature, after which the resulting solution was stirred at the same temperature for 18 hours. Saturated aqueous solution of sodium hydrogen carbonate was poured into the reaction mixture, and then an organic layer was extracted with dichloromethane, filtered via a plastic filter to remove a solid residue and an aqueous solution layer therefrom, and concentrated under reduced pressure. An obtained product was used without an additional purification process (title compound, 0.042 g, 91.7%, yellow oil).

### [Step 5] Synthesis of (S)-2-((7-amino-2-(furan-2-yl)-[1,2,4]triazolo[1,5-a][1,3,5]triazin-5-yl)amino)-1-(4-(2,2,2-trifluoroethyl)piperazin-1-yl)propan-1-one

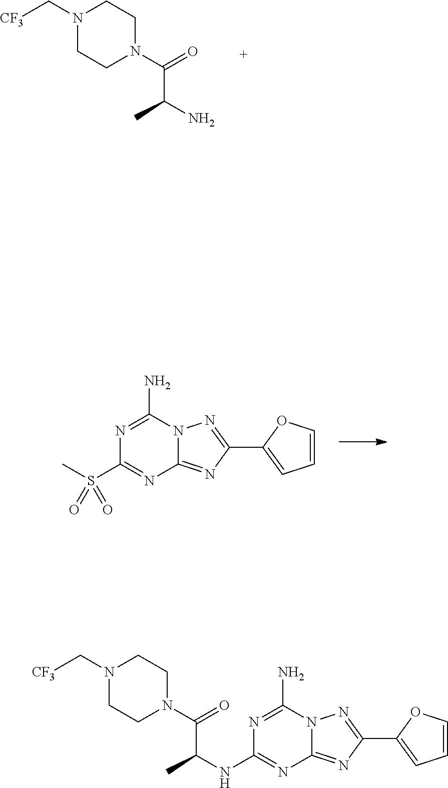 [SMILES: C[C@H](N)C(=O)N1CCN(CC(F)(F)F)CC1 ; CS(=O)(=O)c1nc(N)n2nc(-c3ccco3)nc2n1 ; C[C@H](Nc1nc(N)n2nc(-c3ccco3)nc2n1)C(=O)N1CCN(CC(F)(F)F)CC1]

2-(furan-2-yl)-5-(methylsulfonyl)-[1,2,4]triazolo[1,5-a][1,3,5]triazine-7-amine (0.042 g, 0.150 mmol) prepared in step 4, (S)-2-amino-1-(4-(2,2,2-trifluoroethyl)piperazin-1-yl)propan-1-one (0.036 g, 0.150 mmol) and triethylamine (0.042 mL, 0.300 mmol) were dissolved in dimethylsulfoxide (1 mL) at room temperature, and the resulting solution was stirred at the same temperature for 18 hours. Water was poured into the reaction mixture, and then an organic layer was extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; methanol/dichloromethane=0 to 10%) and concentrated to obtain a title compound (0.010 g, 15.3%) as a white solid form.

¹H NMR (400 MHz, Chloroform-d) δ 8.40 (s, 1H), 8.02 (d, J=8.6 Hz, 1H), 7.61 (s, 1H), 7.21 (s, 1H), 6.59 (s, 1H), 5.39-5.31 (m, 1H), 4.05-3.97 (m, 1H), 3.93-3.85 (m, 1H), 3.74-3.67 (m, 1H), 3.59-3.50 (m, 1H), 3.15-2.98 (m, 2H), 2.93-2.81 (m, 2H), 2.78-2.74 (m, 1H), 2.70-2.62 (m, 1H), 1.44 (d, J=6.8 Hz, 3H); LRMS (ES) m/z 440.4 (M⁺+1).

### Example 4: Synthesis of Compound 4

Example compound 4 was synthesized through substantially the same synthesis method as a synthesis method of example compound 25 except for using (tert-butoxycarbonyl)-L-phenylalanine instead of (tert-butoxycarbonyl)-L-alanine.

¹H NMR (400 MHz, Chloroform-d) δ 9.64 (s, 1H), 8.69 (d, J=9.2 Hz, 1H), 7.62 (s, 1H), 7.32-7.07 (m, 5H), 6.59 (s, 1H), 6.32 (s, 1H), 5.63 (q, J=8.4 Hz, 1H), 3.81-3.71 (m, 1H), 3.68-3.60 (m, 1H), 3.57-3.48 (m, 2H), 3.09 (d, J=7.7 Hz, 2H), 2.91 (q, J=9.0, 8.5 Hz, 2H), 2.70-2.64 (m, 1H), 2.64-2.54 (m, 1H), 2.41 (t, J=9.3 Hz, 1H), 2.04 (t, J=9.6 Hz, 1H); LRMS (ES) m/z 516.3 (M⁺+1).

### Example 64: Synthesis of Compound 64, (4-((7-amino-2-(furan-2-yl)-[1,2,4]triazolo[1,5-a][1,3,5]triazin-5-yl)-L-prolyl)piperazin-1-yl)(2,4-difluorophenyl)methanone

### [Step 1] Synthesis of tert-butyl (S)-2-(piperazin-1-carbonyl)pyrrolidin-1-carboxylate

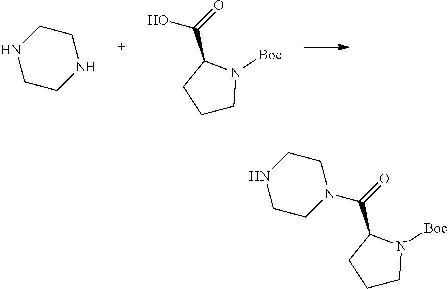 [SMILES: C1CNCCN1 ; CC(C)(C)OC(=O)N1CCC[C@H]1C(=O)O ; CC(C)(C)OC(=O)N1CCC[C@H]1C(=O)N1CCNCC1]

(Tert-butoxycarbonyl)-L-proline (10.763 g, 50.000 mmol), piperazine (12.920 g, 150.000 mmol), 1-ethyl-3-(3-dimethylaminopropyl)carbodiimide hydrochloride (EDC-HCl, 19.170 g, 100.000 mmol), 1H-benzo[d][1,2,3]triazol-1-ol (HOBt, 7.432 g, 55.000 mmol) and N,N-diisopropylethylamine (26.127 mL, 150.000 mmol) were dissolved in dichloromethane (200 mL) at room temperature, after which the resulting solution was stirred at the same temperature for 18 hours. Saturated aqueous solution of ammonium chloride was poured into the reaction mixture, and an organic layer was extracted with dichloromethane. The organic layer was washed with saturated aqueous solution of sodium chloride, dehydrated with anhydrous magnesium sulfate, filtered, and concentrated under reduced pressure. The resulting concentrate was purified via column chromatography (SiO₂, 80 g cartridge; methanol/dichloromethane=0 to 15%) and concentrated to obtain a title compound (6.718 g, 47.4%) as a white solid form.

### [Step 2] Synthesis of tert-butyl (S)-2-(4-(2,4-difluorobenzoyl)piperazin-1-carbonyl)pyrrolidin-1-carboxylate

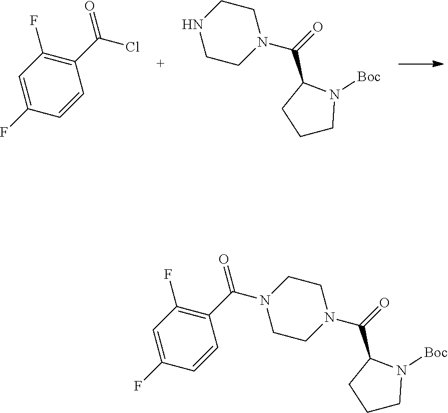 [SMILES: O=C(Cl)c1ccc(F)cc1F ; CC(C)(C)OC(=O)N1CCC[C@H]1C(=O)N1CCNCC1 ; CC(C)(C)OC(=O)N1CCC[C@H]1C(=O)N1CCN(C(=O)c2ccc(F)cc2F)CC1]

Tert-butyl (S)-2-(piperazin-1-carbonyl)pyrrolidin-1-carboxylate (0.283 g, 1.000 mmol) prepared in step 1 and 2,4-difluorobenzoyl chloride (0.124 mL, 1.000 mmol) were dissolved in dichloromethane (5 mL) at room temperature, after which the resulting solution was stirred at the same temperature for 18 hours. Solvent was removed from the reaction mixture under reduced pressure, after which an obtained product was used without an additional purification process (title compound, 0.420 g, 99.2%, light yellow oil).

### [Step 3] Synthesis of (S)-(2,4-difluorophenyl)(4-prolylpiperazin-1-yl)methanone hydrochloride

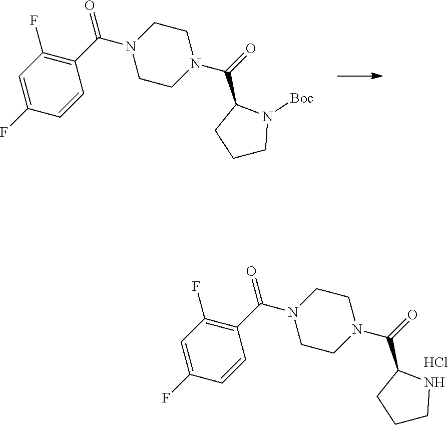 [SMILES: CC(C)(C)OC(=O)N1CCC[C@H]1C(=O)N1CCN(C(=O)c2ccc(F)cc2F)CC1 ; O=C(c1ccc(F)cc1F)N1CCN(C(=O)[C@@H]2CCCN2)CC1 ; Cl]

Tert-butyl (S)-2-(4-(2,4-difluorobenzoyl)piperazin-1-carbonyl)pyrrolidin-1-carboxylate (0.420 g, 0.992 mmol) prepared in step 2 and hydrogen chloride (4.00 M solution in 1,4-dioxane, 0.992 mL, 3.967 mmol) were dissolved in dichloromethane (3 mL) at room temperature, after which the resulting solution was stirred at the same temperature for three hours. Solvent was removed from the reaction mixture under reduced pressure, after which an obtained product was used without an additional purification process (title compound, 0.356 g, 99.8%, white solid).

### [Step 4] Synthesis of (4-((7-amino-2-(furan-2-yl)-[1,2,4]triazolo[1,5-a][1,3,5]triazin-5-yl)-L-prolyl)piperazin-1-yl)(2,4-difluorophenyl)methanone

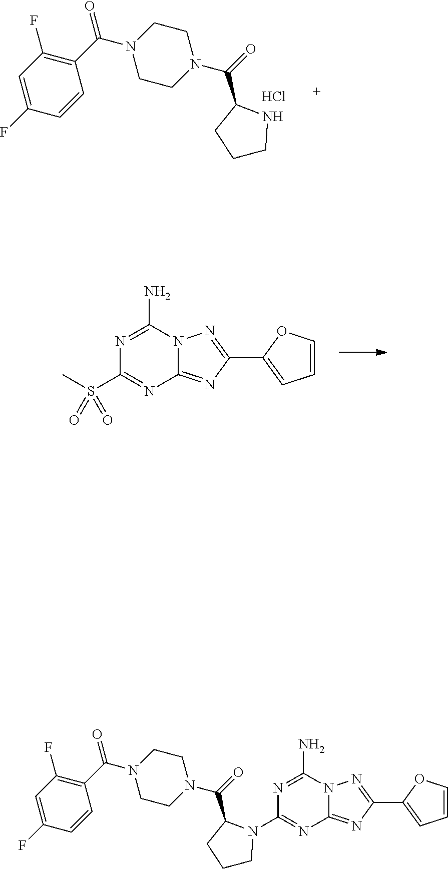 [SMILES: O=C(c1ccc(F)cc1F)N1CCN(C(=O)[C@@H]2CCCN2)CC1 ; Cl ; CS(=O)(=O)c1nc(N)n2nc(-c3ccco3)nc2n1 ; Nc1nc(N2CCC[C@H]2C(=O)N2CCN(C(=O)c3ccc(F)cc3F)CC2)nc2nc(-c3ccco3)nn12]

(S)-(2,4-Difluorophenyl)(4-prolylpiperazin-1-yl)methanone hydrochloride (0.356 g, 0.989 mmol) prepared in step 3, 2-(furan-2-yl)-5-(methylsulfonyl)-[1,2,4]triazolo[1,5-a][1,3,5]triazin-7-amine (0.277 g, 0.989 mmol) and triethylamine (0.414 mL, 2.968 mmol) were dissolved in N,N-dimethylformamide (5 mL) at room temperature, after which the resulting solution was stirred at the same temperature for 18 hours. Solvent was removed from the reaction mixture under reduced pressure, after which the resulting concentrate was purified via column chromatography (SiO₂, 12 g cartridge; methanol/dichloromethane=0 to 5%) and concentrated to obtain a title compound (0.227 g, 43.7%) as a light yellow solid form.

¹H NMR (400 MHz, DMSO-d₆) δ 8.75-8.05 (m, 2H), 7.97-7.80 (m, 1H), 7.74-7.48 (m, 1H), 7.47-7.33 (m, 1H), 7.34-7.14 (m, 1H), 7.13-7.00 (m, 1H), 6.74-6.61 (m, 1H), 5.11-4.88 (m, 1H), 4.03-3.38 (m, 10H), 2.38-2.13 (m, 1H), 2.03-1.77 (m, 3H); LRMS (ES) m/z 524.5 (M⁺+1).

### Example 5: Synthesis of Compound 5

Example compound 5 was synthesized through substantially the same synthesis method as a synthesis method of example compound 64 except for using (tert-butoxycarbonyl)-L-phenylalanine instead of (tert-butoxycarbonyl)-L-proline and using isobutyryl chloride instead of 2,4-difluorobenzoyl chloride.

### Examples 46 and 63

Example compounds 46 and 63 were prepared through substantially the same synthesis method as a synthesis method of example compound 64 except for using benzoyl chloride and 3-hydroxybenzoyl chloride, respectively, instead of 24-difluorobenzoyl chloride.

### Example 99: Synthesis of Compound 99

Example compound 99 was synthesized through substantially the same synthesis method as a synthesis method of Example compound 64 except for using (tert-butoxycarbonyl)-D-proline instead of (tert-butoxycarbonyl)-L-proline and using morpholin-4-carbonyl chloride instead of 2,4-difluorobenzoyl chloride.

Analysis data of each of the compounds prepared as described above are shown in the table below.

TABLE 49

| Example | | |
|---|---|---|
| No. | Compound Names | Analysis Data |
| 5 | (S)-2-((7-amino- | ¹H NMR (400 MHz, Chloroform-d) |
| | 2-(furan-2-yl)- | δ 9.83-9.46 (m, |
| | [1,2,4]triazolo[1,5- | 1H), 8.93-8.65 (m, 1H), |
| | a][1,3,5]triazin- | 7.62 (s, 1H), 7.35-7.10 (m, |
| | 5-yl)amino)-1- | 6H), 6.59 (d, J = 3.4 Hz, |
| | (4-isobutyryl- | 1H), 6.39 (s, 1H), 5.58 (d, J = |
| | piperazin-1-yl)-3- | 10.9 Hz, 1H), 3.82-3.33 |
| | phenylpropan-1-one | (m, 6H), 3.29-2.61 (m, |
| | | 6H), 1.16-1.08 (m, 6H); |
| | | LRMS (ES) m/z 504.4 (M⁺ + |
| | | 1). |
| 46 | 4-((7-amino-2- | ¹H NMR (400 MHz, |
| | (furan-2-yl)- | Chloroform-d) δ 7.60-7.54 (m, |
| | [1,2,4]triazolo[1,5- | 1H), 7.51-7.41 (m, 5H), |
| | a][1,3,5]triazin- | 7.24-7.14 (m, 1H), 6.56 |
| | 5-yl)-L- | (ddd, J = 3.5, 1.8, 0.6 Hz, |
| | prolyl)piperazin-1- | 1H), 6.12 (s, 2H), 5.11-4.90 |
| | yl)(phenyl)methanone | (m, 1H), 4.20-3.36 (m, |
| | | 8H), 2.39-2.09 (m, 2H), |
| | | 2.09-1.94 (m, 2H), 1.82 |
| | | (s, 2H); LRMS (ES) m/z |
| | | 488.5 (M⁺ + 1). |
| 63 | 4-((7-amino-2- | ¹H NMR (400 MHz, |
| | (furan-2-yl)- | Chloroform-d) δ 7.57 (s, 1H), 7.40- |
| | [1,2,4]triazolo[1,5- | 7.32 (m, 1H), 7.25-7.16 |
| | a][1,3,5]triazin- | (m, 1H), 7.05-6.94 (m, |
| | 5-yl)-L- | 3H), 6.56 (s, 1H), 6.11-6.06 |
| | prolyl)piperazin- | (m, 1H), 5.16-4.88 (m, |
| | 1-yl)(3- | 1H), 4.14-3.39 (m, 14H), |
| | methoxy- | 2.42-2.12 (m, 2H), 2.12- |
| | phenyl)methanone | 1.77 (m, 2H); LRMS (ES) |
| | | m/z 518.3 (M⁺ + 1). |
| 99 | 4-((7-amino-2- | ¹H NMR (400 MHz, |
| | (furan-2-yl)- | Chloroform-d) δ 7.63-7.49 (m, |
| | [1,2,4]triazolo[1,5- | 1H), 7.25-7.10 (m, 1H), |
| | a][1,3,5]triazin- | 6.59-6.49 (m, 1H), 6.37- |
| | 5-yl)-D- | 6.17 (m, 2H), 5.13-4.86 |
| | prolyl)piperazin-1-yl) | (m, 1H), 4.04 (d, J = 6.1 Hz, |
| | (morpholino)methanone | OH), 4.00-3.44 (m, 12H), |
| | | 3.44-3.20 (m, 8H), 2.38- |
| | | 2.10 (m, 2H); LRMS (ES) |
| | | m/z 497.4 (M⁺ + 1). |

### Example 71: Synthesis of Compound 71, (S)-(1-(7-amino-2-(furan-2-yl)-[1,2,4]triazolo[1,5-a][1,3,5]triazin-5-yl)pyrrolidin-2-yl)(4-(isopropylsulfonyl)piperazin-1-yl)methanone

### [Step 1] Synthesis of tert-butyl (S)-2-(4-(isopropylsulfonyl)piperazin-1-carbonyl)pyrrolidin-1-carboxylate

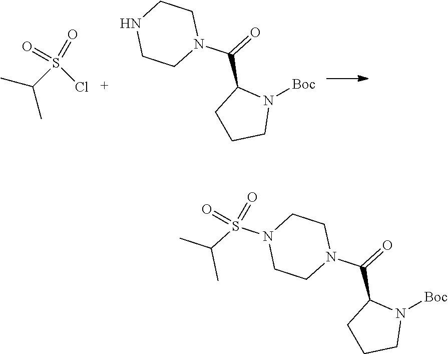

Tert-butyl (S)-2-(piperazin-1-carbonyl)pyrrolidin-1-carboxylate (0.283 g, 1.000 mmol) prepared in step 1 of example 64, propan-2-sulfonyl chloride (0.226 mL, 2.000 mmol) and potassium carbonate (0.415 g, 3.000 mmol) were dissolved in acetonitrile (8 mL) at room temperature, after which the resulting solution was stirred at 50° C. for 18 hours, and then a reaction was finished by lowering the temperature to room temperature. Water was poured into the reaction mixture, and then an organic layer was extracted with dichloromethane, filtered via a plastic filter to remove a solid residue and an aqueous solution layer therefrom, and concentrated under reduced pressure. An obtained product was used without an additional purification process (title compound, 0.770 g, 98.8%, light yellow oil).

### [Step 2] Synthesis of (S)-1-(isopropylsulfonyl)-4-prolylpiperazine hydrochloride

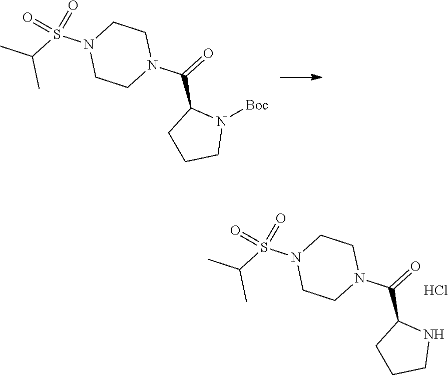 [SMILES: CC(C)S(=O)(=O)N1CCN(C(=O)[C@@H]2CCCN2C(=O)OC(C)(C)C)CC1 ; CC(C)S(=O)(=O)N1CCN(C(=O)[C@@H]2CCCN2)CC1 ; Cl]

Tert-butyl (S)-2-(4-(isopropylsulfonyl)piperazin-1-carbonyl)pyrrolidin-1-carboxylate (0.770 g, 1.977 mmol) prepared in step 1 and hydrogen chloride (4.00 M solution in 1,4-dioxane, 1.977 mL, 7.907 mmol) were dissolved in dichloromethane (4 mL) at room temperature, after which the resulting solution was stirred at the same temperature for 18 hours. Solvent was removed from the reaction mixture under reduced pressure, after which an obtained product was used without an additional purification process (title compound, 0.640 g, 99.4%, light yellow oil).

### [Step 3] Synthesis of (S)-(1-(7-amino-2-(furan-2-yl)-[1,2,4]triazolo[1,5-a][1,3,5]triazin-5-yl)pyrrolidin-2-yl)(4-(isopropylsulfonyl)piperazin-1-yl)methanone

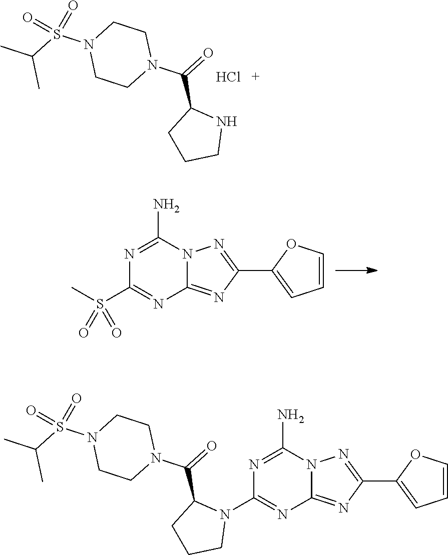

2-(Furan-2-yl)-5-(methylsulfonyl)-[1,2,4]triazolo[1,5-a][1,3,5]triazin-7-amine (0.400 g, 1.427 mmol) prepared in step 2, (S)-1-(isopropylsulfonyl)-4-prolylpiperazine hydrochloride (0.651 g, 1.998 mmol) and sodium hydrogen carbonate (0.360 g, 4.282 mmol) were dissolved in cyclopentyl methyl ether (CPME, 10 mL) at room temperature, after which the resulting solution was stirred at 70° C. for 18 hours to complete the reaction by lowering a temperature to room temperature. Solvent was removed from the reaction mixture under reduced pressure, after which the resulting concentrate was purified via column chromatography (SiO₂, 12 g cartridge; methanol/dichloromethane=0 to 5%) and concentrated to obtain a title compound (0.068 g, 9.7%) as a white solid form.

¹H NMR (400 MHz, DMSO-d₆) δ 8.70-8.09 (m, 2H), 7.90-7.84 (m, 1H), 7.10-6.98 (m, 1H), 6.75-6.63 (m, 1H), 5.09-4.92 (m, 1H), 3.97-3.53 (m, 6H), 3.53-3.38 (m, 3H), 3.31-3.11 (m, 2H), 2.32-2.16 (m, 1H), 2.05-1.78 (m, 3H), 1.36-1.18 (m, 6H); LRMS (ES) m/z 490.6 (M⁺+1).

### Example 6: Synthesis of Compound 6

Example compound 6 was synthesized through substantially the same synthesis method as a synthesis method of example compound 71 except for using benzenesulfonyl chloride instead of propan-2-sulfonyl chloride and using (tert-butoxycarbonyl)-L-phenylalanine instead of (tert-butoxycarbonyl)-L-proline.

### Examples 68, 69, 70 and 72

Example compounds 68, 69, 70 and 72 were synthesized through substantially the same synthesis method as a synthesis method of example compound 71 except for using the compounds of the following table instead of propan-2-sulfonyl chloride as a starting material.

TABLE 50

| Example | Starting |
|---|---|
| No. | Materials |
| 68 | 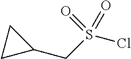 |
| 69 | 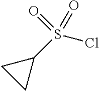 |
| 70 | 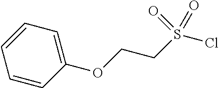 [SMILES: O=S(=O)(Cl)CCOc1ccccc1] |
| 72 | 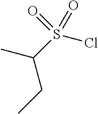 |

Analysis data of each of the compounds prepared as described above are shown in the table below.

TABLE 51

| Example | | |
|---|---|---|
| No. | Compound Names | Analysis Data |
| 72 | ((S)-1-(7-amino- | ¹H NMR (400 MHz, DMSO-d₆) |
| | 2-(furan-2- | δ 8.71-8.10 (m, 2H), |
| | yl)-[1,2,4]triazolo[1,5- | 7.92-7.82 (m, 1H), 7.11-6.96 |
| | a][1,3,5]triazin-5- | (m, 1H), 6.74-6.62 (m, |
| | yl)pyrrolidin- | 1H), 5.10-4.92 (m, 1H), |
| | 2-yl)(4-(sec- | 4.06-3.51 (m, 6H), 3.52-3.41 |
| | butylsulfonyl) | (m, 2H), 3.30-3.02 (m, 4H), |
| | piperazin-1- | 2.36-2.17 (m, 1H), 2.08- |
| | yl)methanone | 1.79 (m, 4H), 1.61-1.36 |
| | | (m, 1H), 1.36-1.15 (m, 3H), |
| | | 1.05-0.88 (m, 2H); LRMS |
| | | (ES) m/z 504.5 (M⁺ + 1). |
| 6 | (S)-2-((7-amino- | ¹H NMR (400 MHz, |
| | 2-(furan-2- | Chloroform-d) δ 9.34 (s, 1H), 8.61 |
| | yl)-[1,2,4]triazolo[1,5- | (d, J = 9.1 Hz, 1H), 7.69 (d, |
| | a][1,3,5]triazin-5- | J = 7.6 Hz, 2H), 7.65 (s, 1H), |
| | yl)amino)-3- | 7.58 (t, J = 7.8 Hz, 1H), 7.45 |
| | phenyl-1-(4- | (t, J = 7.7 Hz, 2H), 7.26 (s, |
| | (phenylsulfonyl) | 1H), 7.14-7.04 (m, 4H), |
| | piperazin- | 7.00-6.92 (m, 1H), 6.62 (s, |
| | 1-yl)propan-1-one | 1H), 6.34 (s, 1H), 5.49-5.41 |
| | | (m, 1H), 4.89-4.65 (m, |
| | | 4H), 3.92-3.83 (m, 1H), 3.61 |
| | | (d, J = 17.5 Hz, 2H), 3.50- |
| | | 3.42 (m, 1H), 3.06-2.94 |
| | | (m, 3H), 2.88-2.82 (m, |
| | | 1H), 2.78-2.74 (m, 1H), 2.43 |
| | | (s, 1H); LRMS (ES) m/z |
| | | 574.4 (M⁺ + 1). |
| 68 | (S)-(1-(7-amino- | ¹H NMR (400 MHz, |
| | 2-(furan-2-yl)- | Chloroform-d) δ 7.59 (dd, J = 1.8, |
| | [1,2,4]triazolo[1,5- | 0.8 Hz, 1H), 7.13 (s, 1H), 6.56 |
| | a][1,3,5]triazin-5- | (dd, J = 3.5, 1.8 Hz, 1H), |
| | yl)pyrrolidin- | 6.04 (s, 2H), 5.01 (ddd, J = |
| | 2-yl)(4- | 53.9, 7.9, 3.3 Hz, 1H), 4.41- |
| | ((cyclopropyl | 4.28 (m, 1H), 4.18 (d, J = 13.4 |
| | methyl)sulfon | Hz, 1H), 3.96-3.66 (m, |
| | yl)piperazin-1- | 4H), 3.64-3.52 (m, 2H), |
| | yl)methanone | 3.25-3.08 (m, 3H), 3.03- |
| | | 2.93 (m, 1H), 2.36-2.19 |
| | | (m, 2H), 2.11-1.95 (m, 2H), |
| | | 1.25-1.12 (m, 1H), 0.80-0.61 |
| | | (m, 2H), 0.45-0.31 (m, |
| | | 2H); LRMS (ES) m/z 502.5 |
| | | (M⁺ + 1). |
| 69 | (S)-(1-(7-amino- | ¹H NMR (400 MHz, |
| | 2-(furan-2- | Chloroform-d) δ 7.61-7.52 (m, |
| | yl)-[1,2,4]triazolo[1,5- | 1H), 7.01 (d, J = 3.4 Hz, 1H), |
| | a][1,3,5]triazin-5- | 6.59-6.51 (m, 1H), 6.15 (s, |
| | yl)pyrrolidin-2-yl)(4- | 2H), 5.11-4.89 (m, 1H), 4.38 |
| | (cyclopropyl- | (d, J = 9.2 Hz, 1H), 4.20 |
| | sulfonyl)pipera | (d, J = 13.7 Hz, 1H), 3.97-3.83 |
| | zin-1-yl)methanone | (m, 2H), 3.79 (dt, J = |
| | | 10.5, 6.6 Hz, 1H), 3.71-3.64 |
| | | (m, 1H), 3.62-3.49 (m, |
| | | 2H), 3.15 (d, J = 8.9 Hz, 2H), |
| | | 2.79 (tt, J = 8.0, 4.9 Hz, |
| | | 1H), 2.38-2.19 (m, 2H), |
| | | 2.11-1.95 (m, 2H), 1.25-1.16 |
| | | (m, 1H), 1.16-1.08 (m, 1H), |
| | | 1.08-0.99 (m, 1H), 0.99- |
| | | 0.86 (m, 1H); LRMS (ES) |
| | | m/z 488.4 (M⁺ + 1). |
| 70 | (S)-(1-(7-amino- | ¹H NMR (400 MHz, |
| | 2-(furan-2- | Chloroform-d) δ 7.62-7.47 (m, |
| | yl)-[1,2,4]triazolo[1,5- | 1H), 7.37-7.18 (m, 2H), |
| | a][1,3,5]triazin-5- | 7.10 (d, J = 3.4 Hz, 1H), 7.06- |
| | yl)pyrrolidin- | 6.96 (m, 1H), 6.96-6.88 |
| | 2-yl)(4-((2- | (m, 1H), 6.88-6.84 (m, 1H), |
| | phenoxyethyl) | 6.58-6.41 (m, 1H), 6.26-5.96 |
| | sulfonyl)piperazin- | (m, 2H), 4.98 (ddd, J = |
| | 1-yl)methanone | 58.6, 8.1, 3.3 Hz, 1H), |
| | | 4.46-4.34 (m, 2H), 4.22 (d, J = |
| | | 12.7 Hz, 1H), 4.12 (d, J = 13.5 |
| | | Hz, 1H), 3.96-3.83 (m, |
| | | 2H), 3.83-3.72 (m, 2H), |
| | | 3.72-3.48 (m, 5H), 3.37- |
| | | 3.18 (m, 2H), 2.34-2.16 (m, |
| | | 2H), 2.09-1.95 (m, 2H); |
| | | LRMS (ES) m/z 568.7 (M⁺ + 1). |

### Example 111: Synthesis of Compound 111, (S)-(1-(7-amino-2-(furan-2-yl)-[1,2,4]triazolo[1,5-a][1,3,5]triazin-5-yl)azetidin-2-yl)(4-butylpiperazin-1-yl)methanone

### [Step 1] Synthesis of tert-butyl (S)-2-(piperazin-1-carbonyl)azetidin-1-carboxylate

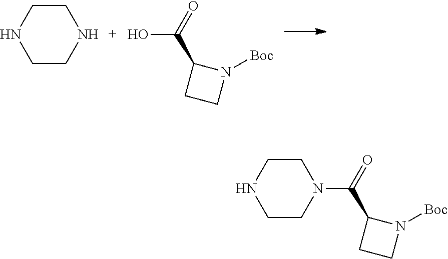

(S)-1-(Tert-butoxycarbonyl)azetidin-2-carboxylic acid (2.012 g, 10.000 mmol), piperazine (2.584 g, 30.000 mmol) and 2,4,6-tripropyl-1,3,5,2,4,6-trioxatriphosphinane 2,4,6-trioxide (T3P, 50.00% solution in EtOAc, 8.918 mL, 15.000 mmol) were dissolved in dichloromethane (40 mL) at room temperature, after which the resulting solution was stirred at the same temperature for 18 hours. Saturated aqueous solution of sodium hydrogen carbonate was poured into the reaction mixture, and an organic layer was extracted with dichloromethane. The organic layer was washed with saturated aqueous solution of sodium chloride, dehydrated with anhydrous magnesium sulfate, filtered, and concentrated under reduced pressure. The resulting concentrate was purified via column chromatography (SiO₂, 40 g cartridge; methanol/dichloromethane=0 to 10%) and concentrated to obtain a title compound (0.508 g, 18.9%) as a colorless oil form.

### [Step 2] Synthesis of tert-butyl (S)-2-(4-butylpiperazin-1-carbonyl)azetidin-1-carboxylate

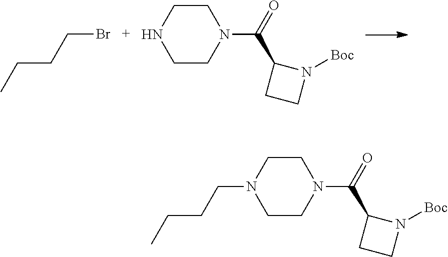

Tert-butyl (S)-2-(piperazin-1-carbonyl)azetidin-1-carboxylate (0.150 g, 0.557 mmol) prepared in step 1, N,N-diisopropylethylamine (0.097 mL, 0.557 mmol) and 1-bromobutane (0.060 mL, 0.557 mmol) were dissolved in acetonitrile (2 mL) at room temperature, after which the resulting solution was stirred at the same temperature for 18 hours. Water was poured into the reaction mixture, and then an organic layer was extracted with ethyl acetate, filtered via a plastic filter to remove a solid residue and an aqueous solution layer therefrom, and concentrated under reduced pressure. An obtained product was used without an additional purification process (title compound, 0.181 g, 99.9%, colorless oil).

### [Step 3] Synthesis of (S)-azetidin-2-yl(4-butylpiperazin-1-yl)methanone hydrochloride

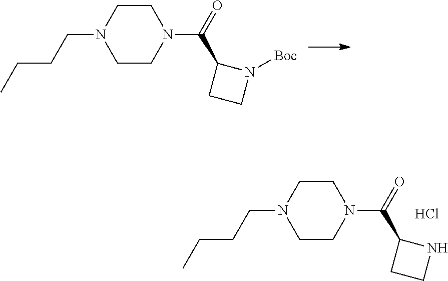 [SMILES: CCCCN1CCN(C(=O)[C@@H]2CCN2C(=O)OC(C)(C)C)CC1 ; CCCCN1CCN(C(=O)[C@@H]2CCN2)CC1 ; Cl]

Tert-butyl (S)-2-(4-butylpiperazin-1-carbonyl)azetidin-1-carboxylate (0.181 g, 0.556 mmol) prepared in step 2 and hydrogen chloride (4.00 M solution in 1,4-dioxane, 0.556 mL, 2.225 mmol) were dissolved in dichloromethane (3 mL) at room temperature, after which the resulting solution was stirred at 40° C. for four hours to complete the reaction by lowering a temperature to room temperature. Solvent was removed from the reaction mixture under reduced pressure, after which an obtained product was used without an additional purification process (title compound, 0.145 g, 99.6%, white solid).

### [Step 4] Synthesis of (S)-(1-(7-amino-2-(furan-2-yl)-[1,2,4]triazolo[1,5-a][1,3,5]triazin-5-yl)azetidin-2-yl)(4-butylpiperazin-1-yl)methanone

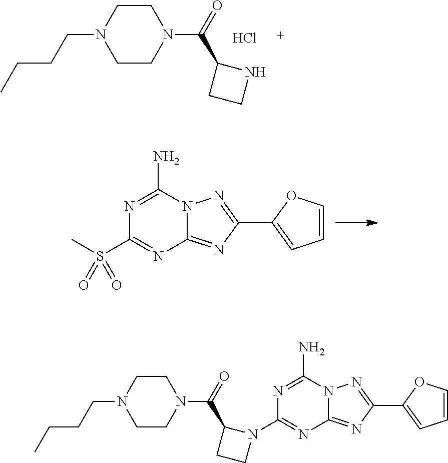 [SMILES: CCCCN1CCN(C(=O)[C@@H]2CCN2)CC1 ; Cl ; CS(=O)(=O)c1nc(N)n2nc(-c3ccco3)nc2n1 ; CCCCN1CCN(C(=O)[C@@H]2CCN2c2nc(N)n3nc(-c4ccco4)nc3n2)CC1]

(S)-Azetidin-2-yl(4-butylpiperazin-1-yl)methanone hydrochloride (0.145 g, 0.554 mmol) prepared in step 3, 2-(furan-2-yl)-5-(methylsulfonyl)-[1,2,4]triazolo[1,5-a][1,3,5]triazin-7-amine (0.155 g, 0.554 mmol) and sodium hydrogen carbonate (0.140 g, 1.662 mmol) were dissolved in cyclopentylmethyl ether (CPME, 4 mL) at room temperature, after which the resulting solution was stirred at 50° C. for 18 hours to complete the reaction by lowering a temperature to room temperature. Solvent was removed from the reaction mixture under reduced pressure, after which the resulting concentrate was purified via column chromatography (SiO₂, 12 g cartridge; methanol/dichloromethane=0 to 10%) and concentrated to obtain a title compound (0.135 g, 57.5%) as a white solid form.

¹H NMR (400 MHz, DMSO-d₆) δ 8.78-8.01 (m, 2H), 7.92-7.82 (m, 1H), 7.12-6.99 (m, 1H), 6.73-6.62 (m, 1H), 5.20 (dd, 1H), 4.10-3.89 (m, 2H), 3.86-3.14 (m, 5H), 2.74-2.55 (m, 1H), 2.46-2.02 (m, 6H), 1.50-1.35 (m, 2H), 1.35-1.19 (m, 2H), 0.88 (t, J=7.3 Hz, 3H); LRMS (ES) m/z 426.6 (M⁺+1).

### Examples 17 and 18

Example compounds 17 and 18 were each synthesized through substantially the same synthesis method as a synthesis method of example compound 111 except for using tert-butyl (S)-(1-oxo-3-phenyl-1-(piperazin-1-yl)propan-2-yl)carbamate instead of tert-butyl (S)-2-(piperazin-1-carbonyl)azetidin-1-carboxylate and using 1-(bromomethyl)-3-fluorobenzene and 1-(bromomethyl)-4-fluorobenzene, respectively, instead of 1-bromobutane.

### Examples 21, 22, 23 and 24

Example compounds 21, 22, 23 and 24 were each synthesized through substantially the same synthesis method as a synthesis method of example compound 111 except for using tert-butyl (S)-(1-oxo-1-(piperazin-1-yl)propan-2-yl)carbamate instead of tert-butyl (S)-2-(piperazin-1-carbonyl)azetidin-1-carboxylate and using the compounds of the following table instead of 1-bromobutane as a starting material.

TABLE 52

| Example | |
|---|---|
| No. | Starting Materials |
| 21 | 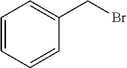 |
| 22 | 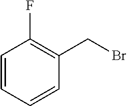 |
| 23 | 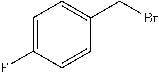 |
| 24 | 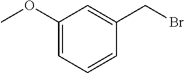 |

### Example 45: Synthesis of Compound 45

Example compound 45 was synthesized through substantially the same synthesis method as a synthesis method of example compound 18 except for using tert-butyl (S)-2-(piperazin-1-carbonyl)pyrrolidin-1-carboxylate instead of tert-butyl (S)-(1-oxo-3-phenyl-1-(piperazin-1-yl)propan-2-yl)carbamate.

### Example 47: Synthesis of Compound 47

Example compound 47 was synthesized through substantially the same synthesis method as a synthesis method of example compound 111 except for using tert-butyl (S)-2-(piperazin-1-carbonyl)pyrrolidin-1-carboxylate instead of tert-butyl (S)-2-(piperazin-1-carbonyl)azetidin-1-carboxylate.

### Example 66: Synthesis of Compound 66

Example compound 66 was synthesized through substantially the same synthesis method as a synthesis method of example compound 111 except for using tert-butyl (S)-2-(piperazin-1-carbonyl)pyrrolidin-1-carboxylate instead of tert-butyl (S)-2-(piperazin-1-carbonyl)azetidin-1-carboxylate and using (1-bromoethyl)benzene instead of 1-bromobutane.

### Example 101: Synthesis of Compound 101

Example compound 101 was synthesized through substantially the same synthesis method as a synthesis method of example compound 111 except for using tert-butyl (R)-2-(piperazin-1-carbonyl)pyrrolidin-1-carboxylate instead of tert-butyl (S)-2-(piperazin-1-carbonyl)azetidin-1-carboxylate.

Analysis data of each of the compounds prepared as described above are shown in the table below.

TABLE 53

| Example | | |
|---|---|---|
| No. | Compound Names | Analysis Data |
| 17 | (S)-2-((7-amino- | ¹H NMR (400 MHz, |
| | 2-(furan-2- | Chloroform-d) δ 9.64 (s, 1H), |
| | yl)-[1,2,4]triazolo[1,5- | 8.66 (d, J = 9.3 Hz, 1H), |
| | a][1,3,5]triazin- | 7.62 (s, 1H), 7.33-7.12 (m, |
| | 5-yl)amino)-1- | 7H), 7.09-6.91 (m, 3H), |
| | (4-(3-fluoro- | 6.59 (s, 1H), 6.30 (s, 1H), |
| | benzyl)piperazin- | 5.70-5.60 (m, 1H), 3.82-3.72 |
| | 1-yl)-3- | (m, 1H), 3.66-3.57 |
| | phenylpropan-1-one | (m, 1H), 3.54-3-46 (m, 2H), |
| | | 3.41 (s, 2H), 3.07 (d, J = |
| | | 7.7 Hz, 2H), 2.46 (s, 1H), |
| | | 2.38 (s, 1H), 2.18 (s, 1H), |
| | | 1.87 (s, 1H); LRMS (ES) |
| | | m/z 542.5 (M⁺ + 1). |
| 18 | (S)-2-((7-amino- | ¹H NMR (400 MHz, |
| | 2-(furan-2- | Chloroform-d) δ 9.57 (s, 1H), 8.61 |
| | yl)-[1,2,4]triazolo[1,5- | (d, J = 9.3 Hz, 1H), 7.62 |
| | a][1,3,5]triazin- | (s, 1H), 7.31-7.07 (m, 8H), |
| | 5-yl)amino)-1- | 7.01 (t, J = 8.3 Hz, 2H), 6.59 |
| | (4-(4-fluoro- | (s, 1H), 6.28 (s, 1H), 5.65 |
| | benzyl)piperazin- | (d, J = 8.9 Hz, 1H), 3.81-3.73 |
| | 1-yl)-3- | (m, 1H), 3.59 (s, 1H), |
| | phenylpropan-1-one | 3.53-3.44 (m, 2H), 3.38 (s, |
| | | 2H), 3.07 (d, J = 7.6 Hz, |
| | | 2H), 2.44 (s, 1H), 2.36 (s, |
| | | 1H), 2.16 (s, 1H), 1.86 (s, |
| | | 1H); LRMS (ES) m/z 542.5 |
| | | (M⁺ + 1). |
| 21 | (S)-2-((7-amino- | ¹H NMR (400 MHz, |
| | 2-(furan-2- | Chloroform-d) δ 9.15-8.66 (m, |
| | yl)-[1,2,4]triazolo[1,5- | 1H), 8.20 (d, J = 8.8 Hz, 1H), |
| | a][1,3,5]triazin- | 7.61 (s, 1H), 7.33 (d, J = |
| | 5-yl)amino)-1- | 20.9 Hz, 5H), 7.24-7.18 (m, |
| | (4-benzylpiperazin-1- | 1H), 6.61-6.50 (m, 1H), |
| | yl)propan-1-one | 6.45-6.03 (m, 1H), 5.43-5.35 |
| | | (m, 1H), 4.02-3.95 |
| | | (m, 1H), 3.88-3.64 (m, 2H), |
| | | 3.64-3.43 (m, 1H), |
| | | 2.55-2.36 (m, 2H), 1.42 |
| | | (s, 3H); LRMS (ES) m/z |
| | | 448.4 (M⁺ + 1). |
| 22 | (S)-2-((7-amino- | ¹H NMR (400 MHz, |
| | 2-(furan-2- | Chloroform-d) δ 8.18 (d, J = 8.8 |
| | yl)-[1,2,4]triazolo[1,5- | Hz, 1H), 7.31-7.17 (m, 3H), |
| | a][1,3,5]triazin- | 7.17-7.00 (m, 3H), 6.58 |
| | 5-yl)amino)-1- | (s, 1H), 6.45-6.08 (m, 1H), |
| | (4-(2-fluoro- | 2.74-2.68 (m, 1H), 2.57- |
| | benzyl)piperazin- | 2.40 (m, 3H), 9.16-8.68 (m, |
| | 1-yl)propan-1-one | 1H), 7.60 (s, 1H), 7.39 |
| | | (t, J = 7.6 Hz, 1H), 5.44-5.32 |
| | | (m, 1H), 3.99 (d, J = |
| | | 12.8 Hz, 1H), 3.83-3.53 (m, |
| | | 3H), 1.41 (d, J = 6.9 Hz, |
| | | 3H); LRMS (ES) m/z 466.4 (M⁺ + 1). |
| 23 | (S)-2-((7-amino- | ¹H NMR (400 MHz, |
| | 2-(furan-2- | Chloroform-d) δ 9.08-8.52 (m, |
| | yl)-[1,2,4]triazolo[1,5- | 1H), 8.17 (d, J = 8.8 Hz, 1H), |
| | a][1,3,5]triazin- | 7.61 (s, 1H), 7.36-7.26 |
| | 5-yl)amino)-1- | (m, 4H), 7.20 (s, 1H), 7.03 |
| | (4-(4-fluoro- | (td, J = 8.8, 2.0 Hz, 3H), |
| | benzyl)piperazin- | 6.59 (s, 1H), 6.50-6.04 (m, |
| | 1-yl)propan-1-one | 1H), 5.38 (t, J = 7.8 Hz, |
| | | 1H), 4.03-3.95 (m, 1H), |
| | | 3.84-3.79 (m, 1H), 3.73- |
| | | 3.67 (m, 1H), 3.61-3.48 |
| | | (m, 3H), 2.62-2.36 (m, |
| | | 4H), 1.42 (d, J = 6.8 Hz, 3H); |
| | | LRMS (ES) m/z 466.4 |
| | | (M⁺ + 1). |
| 24 | (S)-2-((7-amino- | ¹H NMR (400 MHz, |
| | 2-(furan-2- | Chloroform-d) δ 8.82 (s, 1H), |
| | yl)-[1,2,4]triazolo[1,5- | 8.17 (d, J = 8.7 Hz, 1H), |
| | a][1,3,5]triazin- | 7.61 (s, 1H), 7.31-7.15 (m, |
| | 5-yl)amino)-1- | 3H), 6.96-6.88 (m, 3H), |
| | (4-(3- | 6.84 (d, J = 8.5 Hz, 1H), |
| | methoxy- | 6.58 (s, 1H), 6.28 (s, 1H), |
| | benzyl)piperazin-1- | 5.39 (t, J = 7.8 Hz, 1H), 4.01 |
| | yl)propan-1-one | -3.93 (m, 1H), 3.86-3.77 |
| | | (m, 5H), 3.64-3.48 (m, |
| | | 3H), 2.62-2.37 (m, 4H), |
| | | 1.42 (d, J = 6.8 Hz, 3H); |
| | | LRMS (ES) m/z 478.4 (M⁺ + 1). |
| 45 | (S)-(1-(7-amino- | ¹H NMR (400 MHz, |
| | 2-(furan-2- | Chloroform-d) δ 7.59-7.52 (m, |
| | yl)-[1,2,4]triazolo[1,5- | 1H), 7.36-7.25 (m, 2H), |
| | a][1,3,5]triazin-5- | 7.24-7.14 (m, 1H), 7.09- |
| | yl)pyrrolidin- | 6.99 (m, 2H), 6.58-6.51 |
| | 2-yl)(4-(4- | (m, 1H), 6.33-5.77 (m, 2H), |
| | fluoro- | 5.13-4.85 (m, 1H), 3.94-3.82 |
| | benzyl)piperazin-1- | (m, 1H), 3.81-3.70 |
| | yl)methanone | (m, 2H), 3.66-3.45 (m, 5H), |
| | | 2.84-2.73 (m, 1H), |
| | | 2.58-2.32 (m, 3H), 2.32-2.08 |
| | | (m, 2H), 2.04-1.96 |
| | | (m, 2H); LRMS (ES) m/z 492.5 |
| | | (M⁺ + 1). |
| 47 | (S)-(1-(7-amino- | ¹H NMR (400 MHz, |
| | 2-(furan-2- | Chloroform-d) δ 7.60-7.51 (m, |
| | yl)-[1,2,4]triazolo[1,5- | 1H), 7.17 (dt, J = 29.9, 2.6 |
| | a][1,3,5]triazin-5- | Hz, 1H), 6.58-6.50 (m, |
| | yl)pyrrolidin-2-yl)(4- | 1H), 6.43-6.21 (m, 1H), 6.08 |
| | butylpiperazin-1- | (s, 1H), 5.14-4.82 (m, |
| | yl) methanone | 1H), 3.98-3.46 (m, 6H), |
| | | 2.64-2.34 (m, 6H), 2.35- |
| | | 2.23 (m, 1H), 2.17-2.12 (m, |
| | | 1H), 2.06-1.95 (m, 2H), |
| | | 1.62-1.44 (m, 2H), 1.44-1.30 |
| | | (m, 2H), 0.94 (tt, J = |
| | | 6.7, 3.0 Hz, 3H); LRMS |
| | | (ES) m/z 440.5 (M⁺ + 1). |
| 66 | (S)-1-(7-amino- | ¹H NMR (400 MHz, |
| | 2-(furan-2- | Chloroform-d) δ 7.60-7.54 (m, |
| | yl)-[1,2,4]triazolo[1,5- | 1H), 7.41-7.25 (m, 6H), |
| | a][1,3,5]triazin-5- | 7.24-7.13 (m, 1H), 6.59- |
| | yl)pyrrolidin- | 6.53 (m, 1H), 6.11 (s, 1H), |
| | 2-yl)(4-(1- | 5.11-4.79 (m, 1H), 3.94- |
| | phenylethyl) | 3.33 (m, 7H), 2.95-2.66 |
| | piperazin-1- | (m, 1H), 2.59-2.48 (m, |
| | yl)methanone | 2H), 2.44-2.32 (m, 1H), |
| | | 2.31-2.23 (m, 1H), 2.22- |
| | | 2.07 (m, 1H), 2.03-1.94 (m, |
| | | 2H), 1.48-1.36 (m, 3H); |
| | | LRMS (ES) m/z 488.5 (M⁺ + 1). |
| 101 | (R)-(1-(7-amino- | ¹H NMR (400 MHz, |
| | 2-(furan-2- | Chloroform-d) δ 7.60-7.53 (m, |
| | yl)-[1,2,4]triazolo[1,5- | 1H), 7.18-7.12 (m, 1H), |
| | a][1,3,5]triazin-5- | 6.59-6.51 (m, 1H), 6.25- |
| | yl)pyrrolidin-2-yl)(4- | 5.92 (m, 2H), 5.14-4.84 |
| | butylpiperazin-1- | (m, 1H), 3-97-3.46 (m, |
| | yl) methanone | 6H), 2.70-2.09 (m, 8H), |
| | | 2.09-1.97 (m, 2H), 1.62- |
| | | 1.45 (m, 2H), 1.45-1.29 |
| | | (m, 2H), 1.00-0.91 (m, 3H); |
| | | LRMS (ES) m/z 440.3 (M⁺ + 1). |

### Example 61: Synthesis of Compound 61, 4-((7-amino-2-(furan-2-yl)-[1,2,4]triazolo[1,5-a][1,3,5]triazin-5-yl)-L-prolyl)-N-(m-tolyl)piperazin-1-carboxamide

### [Step 1] Tert-butyl (S)-2-(4-(m-tolylcarbamoyl)piperazin-1-carbonyl)pyrrolidin-1-carboxylate

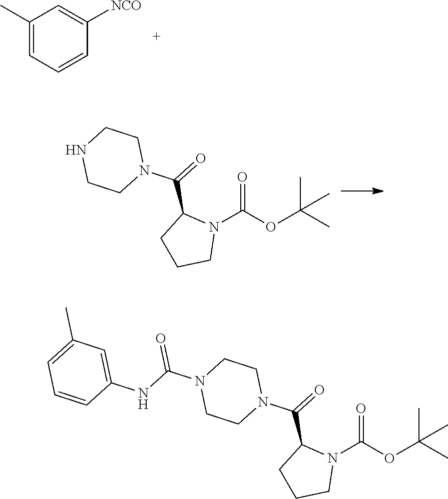

Tert-butyl (S)-2-(piperazin-1-carbonyl)pyrrolidin-1-carboxylate (0.300 g, 1.059 mmol) prepared in step 1 of example 64 and 1-isocyanato-3-methylbenzene (0.141 g, 1.059 mmol) were dissolved in diethyl ether (2 mL) at room temperature, after which the resulting solution was stirred at the same temperature for 18 hours. Water was poured into the reaction mixture, and then an organic layer was extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; methanol/dichloromethane=0 to 5%) and concentrated to obtain a title compound (0.198 g, 44.9%) as a white solid form.

### [Step 2] Synthesis of (S)-4-prolyl-N-(m-tolyl)piperazin-1-carboxamide

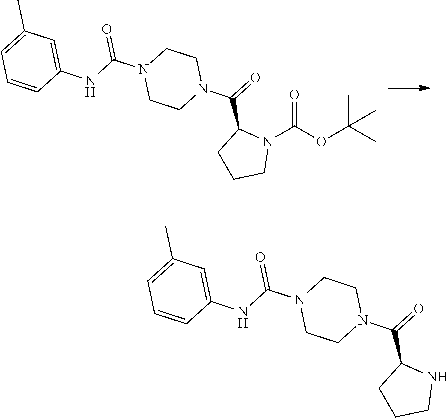 [SMILES: Cc1cccc(NC(=O)N2CCN(C(=O)[C@@H]3CCCN3C(=O)OC(C)(C)C)CC2)c1 ; Cc1cccc(NC(=O)N2CCN(C(=O)[C@@H]3CCCN3)CC2)c1]

Tert-butyl (S)-2-(4-(m-tolylcarbamoyl)piperazin-1-carbonyl)pyrrolidin-1-carboxylate (0.198 g, 0.475 mmol) prepared in step 1 and hydrochloric acid (4.00 M solution in dioxane, 0.594 mL, 2.377 mmol) were mixed, after which the resulting mixture was stirred at room temperature and stirred at the same temperature for 18 hours. Saturated aqueous solution of sodium hydrogen carbonate was poured into the reaction mixture, and then an organic layer was extracted with dichloromethane, filtered via a plastic filter to remove a solid residue and an aqueous solution layer therefrom, and concentrated under reduced pressure. An obtained product was used without an additional purification process (title compound, 0.098 g, 65.2%, light brown oil).

### [Step 3] Synthesis of 4-((7-amino-2-(furan-2-yl)-[1,2,4]triazolo[1,5-a][1,3,5]triazin-5-yl)-L-prolyl)-N-(m-tolyl)piperazin-1-carboxamide

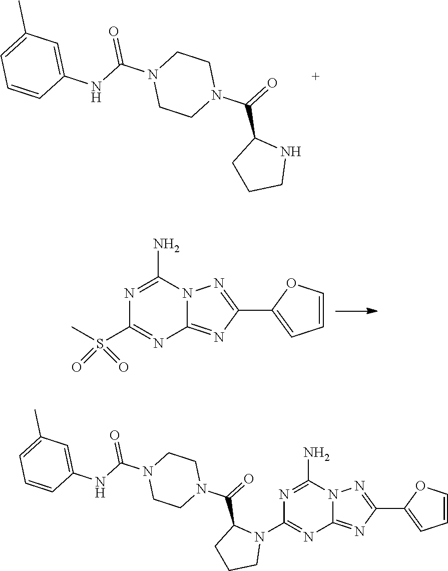

2-(Furan-2-yl)-5-(methylsulfonyl)-[1,2,4]triazolo[1,5-a][1,3,5]triazine-7-amine (0.050 g, 0.178 mmol) prepared in step 2, (S)-4-prolyl-N-(m-tolyl)piperazin-1-carboxamide (0.056 g, 0.178 mmol) and triethylamine (0.050 mL, 0.357 mmol) were dissolved in dimethylsulfoxide (1 mL) at room temperature, after which the resulting solution was stirred for 18 hours at the same temperature. Water was poured into the reaction mixture, and then an organic layer was extracted with dichloromethane, filtered via a plastic filter to remove a solid residue and an aqueous solution layer therefrom, and concentrated under reduced pressure. The resulting concentrate was purified via column chromatography (SiO₂, 4 g cartridge; methanol/dichloromethane=0 to 5%) and concentrated to obtain a title compound (0.017 g, 18.4%) as a white solid form.

¹H NMR (400 MHz, Chloroform-d) δ 7.58-7.45 (m, 1H), 7.27-7.20 (m, 1H), 7.21-7.11 (m, 3H), 7.08-6.91 (m, 1H), 6.90-6.83 (m, 1H), 6.55-6.47 (m, 1H), 6.10 (s, 0H), 5.03-4.81 (m, 1H), 3.97-3.36 (m, 10H), 2.32 (d, J=8.8 Hz, 4H), 2.28-2.11 (m, 1H), 2.08-1.85 (m, 2H); LRMS (ES) m/z 517.2 (M⁺+1).

### Example 100: Synthesis of Compound 100

Example compound 100 was synthesized through substantially the same synthesis method as a synthesis method of example compound 61 except for using tert-butyl (R)-2-(piperazin-1-carbonyl)pyrrolidin-1-carboxylate instead of tert-butyl (S)-2-(piperazin-1-carbonyl)pyrrolidin-1-carboxylate and using isocyanatobenzene instead of 1-isocyanato-3-methylbenzene.

### Examples 62, 115, 116 and 117

Example compounds 62, 115, 116 and 117 were each synthesized through substantially the same synthesis method as a synthesis method of example compound 61 except for using the compounds of the following table instead of 1-isocyanato-3-methylbenzene as a starting material.

TABLE 54

| Example | |
|---|---|
| No. | Starting Materials |
| 62 | 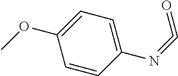 |
| 115 | 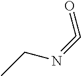 |
| 116 | 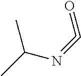 |
| 117 | 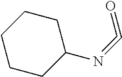 |

Analysis data of each of the compounds prepared as described above are shown in the table below.

TABLE 55

| Example | | |
|---|---|---|
| No. | Compound Names | Analysis Data |
| 62 | 4-((7-amino- | ¹H NMR (400 MHz, |
| | 2-(furan-2-yl)- | Chloroform-d) δ 7.59-7.49 (m, |
| | [1,2,4]triazolo[1,5- | 1H), 7.36-7.30 (m, 2H), |
| | a][1,3,5]triazin- | 7.22-7.14 (m, 1H), 7.06- |
| | 5-yl)-L-prolyl)- | 6.96 (m, 1H), 6.90-6.78 |
| | N-(4-methoxyphenyl) | (m, 3H), 6.52 (ddd, J = 5.2, |
| | piperazin- | 3.4, 1.8 Hz, 1H), 6.20-6.03 |
| | 1-carboxamide | (m, 1H), 5.04-4.82 (m, |
| | | 1H), 4.02-3.34 (m, 13H), |
| | | 2.37-1.83 (m, 5H); LRMS |
| | | (ES) m/z 533.3 (M⁺ + 1). |
| 100 | 4-((7-amino- | ¹H NMR (400 MHz, |
| | 2-(furan-2-yl)- | Chloroform-d) δ 7.57-7.52 (m, |
| | [1,2,4]triazolo[1,5- | 1H), 7.48-7.37 (m, 3H), |
| | a][1,3,5]triazin- | 7.37-7.31 (m, 1H), 7.28- |
| | 5-yl)amino)-D- | 7.20 (m, 2H), 7.20-7.09 |
| | prolyl)-N- | (m, 1H), 7.08-6.97 (m, |
| | phenylpiperazin-1- | 1H), 6.53-6.43 (m, 1H), |
| | carboxamide | 6.38-6.17 (m, 1H), 4.94- |
| | | 4.78 (m, 1H), 4.14 (q, J = |
| | | 7.1 Hz, OH), 3.92-3.27 (m, |
| | | 10H), 2.34-2.14 (m, 2H), |
| | | 2.05-1.85 (m, 2H); |
| | | LRMS (ES) m/z 503.4 (M⁺ + 1). |
| 115 | 4-((7-amino- | ¹H NMR (400 MHz, |
| | 2-(furan-2-yl)- | Chloroform-d) δ 7.60-7.51 (m, |
| | [1,2,4]triazolo[1,5- | 1H), 7.22-7.15 (m, 1H), |
| | a][1,3,5]triazin- | 6.59 (s, 1H), 6.54 (s, 1H), |
| | 5-yl)-L-prolyl)- | 6.16 (s, 1H), 4.97 (ddd, |
| | N-ethylpiperidin- | J = 49.6, 8.4, 3.2 Hz, 1H), 4.69 |
| | 1-carboxamide | (s, 1H), 3.96-3.15 (m, 12H), |
| | | 2.36-2.14 (m, 2H), |
| | | 2.08-1.94 (m, 2H), 1.23-1.11 |
| | | (m, 3H); LRMS (ES) |
| | | m/z 455.6 (M⁺ + 1). |
| 116 | 4-((7-amino- | ¹H NMR (400 MHz, |
| | 2-(furan-2-yl)- | Chloroform-d) δ 7.60-7.54 (m, |
| | [1,2,4]triazolo[1,5- | 1H), 7.24-7.16 (m, 1H), |
| | a][1,3,5]triazin- | 6.59-6.51 (m, 1H), 6.32 (s, |
| | 5-yl)-L-prolyl)- | 1H), 6.18 (s, 1H), 5.11-4.87 |
| | N-isopropylpiperazin-1- | (m, 1H), 4.47-4.38 (m, |
| | carboxamide | 1H), 4.08-3.22 (m, 11H), |
| | | 2.25 (dddd, J = 28.8, 16.0, |
| | | 10.6, 5.8 Hz, 2H), 2.06-1.95 |
| | | (m, 3H), 1.24-1.15 (m, |
| | | 6H); LRMS (ES) m/z 469.7 |
| | | (M⁺ + 1). |
| 117 | 4-((7-amino- | ¹H NMR (400 MHz, |
| | 2-(furan-2-yl)- | Chloroform-d) δ 7.59-7.52 (m, |
| | [1,2,4]triazolo[1,5- | 1H), 7.23-7.15 (m, 1H), |
| | a][1,3,5]triazin- | 6.58-6.50 (m, 1H), 6.48 (s, |
| | 5-yl)-L-prolyl)- | 1H), 6.36 (s, 1H), 5.09-4.75 |
| | N-cyclohexylpiperazin- | (m, 1H), 4.58-4.51 (m, |
| | 1-carboxamide | 1H), 3.95-3.21 (m, 11H), |
| | | 2.39-2.10 (m, 4H), 1.98 |
| | | (h, J = 7.0 Hz, 2H), 1.76-1.67 |
| | | (m, 2H), 1.67-1.58 |
| | | (m, 1H), 1.43-1.29 (m, 2H), |
| | | 1.24-1.03 (m, 3H); |
| | | LRMS (ES) m/z 509.7 (M⁺ + 1). |

### Example 82: Synthesis of Compound 82, (4-((7-amino-2-(furan-2-yl)-[1,2,4]triazolo[1,5-a][1,3,5]triazin-5-yl)-L-prolyl)piperazin-1-yl)(3-(4-methylpiperazin-1-yl)phenyl)methanone

### [Step 1] Synthesis of tert-butyl (S)-2-(4-(3-(4-methylpiperazin-1-yl)benzoyl)piperazin-1-carbonyl)pyrrolidin-1-carboxylate

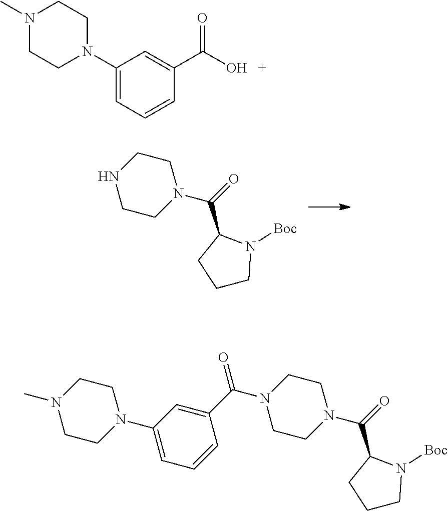

Tert-butyl (S)-2-(piperazin-1-carbonyl)pyrrolidin-1-carboxylate (0.150 g, 0.529 mmol) prepared in step 1 of example 64, 3-(4-methylpiperazin-1-yl)benzoic acid (0.117 g, 0.529 mmol), triethylamine (0.221 mL, 1.588 mmol) and 2,4,6-tripropyl-1,3,5,2,4,6-trioxatriphosphinane 2,4,6-trioxide (T3P, 50.00% solution in EtOAc, 0.945 mL, 1.588 mmol) were dissolved in dichloromethane (2 mL) at room temperature, after which the resulting solution was stirred at the same temperature for three hours. Saturated aqueous solution of sodium hydrogen carbonate was poured into the reaction mixture, and then an organic layer was extracted with dichloromethane, filtered via a plastic filter to remove a solid residue and an aqueous solution layer therefrom, and concentrated under reduced pressure. An obtained product was used without an additional purification process (title compound, 0.250 g, 97.3%, light yellow oil).

### [Step 2] Synthesis of (S)-(3-(4-methylpiperazin-1-yl)phenyl)(4-prolylpiperazin-1-yl)methanone hydrochloride

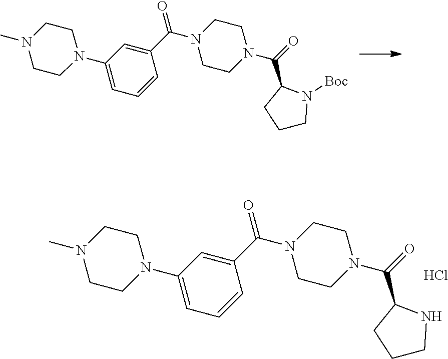 [SMILES: CN1CCN(c2cccc(C(=O)N3CCN(C(=O)[C@@H]4CCCN4C(=O)OC(C)(C)C)CC3)c2)CC1 ; CN1CCN(c2cccc(C(=O)N3CCN(C(=O)[C@@H]4CCCN4)CC3)c2)CC1 ; Cl]

Tert-butyl (S)-2-(4-(3-(4-methylpiperazin-1-yl)benzoyl)piperazin-1-carbonyl)pyrrolidin-1-carboxylate (0.250 g, 0.515 mmol) prepared in step 1 and hydrogen chloride (4.00 M solution in 1,4-dioxane, 0.515 mL, 2.059 mmol) were dissolved in dichloromethane (2 mL) at room temperature, after which the resulting solution was stirred at 40° C. for two hours to complete the reaction by lowering a temperature to room temperature. Solvent was removed from the reaction mixture under reduced pressure, after which an obtained product was used without an additional purification process (title compound, 0.210 g, 96.7%, light yellow solid).

### [Step 3] Synthesis of (4-((7-amino-2-(furan-2-yl)-[1,2,4]triazolo[1,5-a][1,3,5]triazin-5-yl)-L-prolyl)piperazin-1-yl)(3-(4-methylpiperazin-1-yl)phenyl)methanone

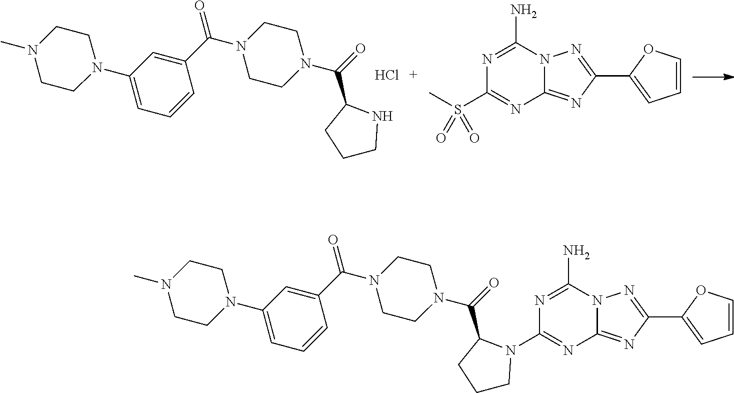

(S)-(3-(4-Methylpiperazin-1-yl)phenyl)(4-prolylpiperazin-1-yl)methanone hydrochloride (0.210 g, 0.498 mmol) prepared in step 2, 2-(furan-2-yl)-5-(methylsulfonyl)-[1,2,4]triazolo[1,5-a][1,3,5]triazin-7-amine (0.126 g, 0.448 mmol) and sodium hydrogen carbonate (0.125 g, 1.493 mmol) were dissolved in cyclopentylmethyl ether (CPME, 3 mL) at room temperature, after which the resulting solution was stirred at 70° C. for 18 hours to complete the reaction by lowering a temperature to room temperature. Solvent was removed from the reaction mixture under reduced pressure, after which the resulting concentrate was purified via column chromatography (SiO₂, 12 g cartridge; methanol/dichloromethane=0 to 5%) and concentrated to obtain a title compound (0.042 g, 14.5%) as a white solid form.

¹H NMR (400 MHz, DMSO-d₆) δ 8.60-8.09 (m, 2H), 7.90-7.85 (m, 1H), 7.36-7.23 (m, 1H), 7.10-7.01 (m, 2H), 6.99-6.89 (m, 1H), 6.82 (s, 1H), 6.73-6.65 (m, 1H), 5.00 (s, 1H), 4.04-3.48 (m, 10H), 3.26-2.93 (m, 6H), 2.38-2.14 (m, 4H), 2.03-1.77 (m, 3H), 1.17 (t, J=7.3 Hz, 2H); LRMS (ES) m/z 586.6 (M⁺+1).

### Examples 83, 102, 103, 104, 105 and 106

Example compounds 83, 102, 103, 104, 105 and 106 were each synthesized through substantially the same synthesis method as a synthesis method of example compound 82 except for using the starting materials below instead of tert-butyl (S)-2-(piperazin-1-carbonyl)pyrrolidin-1-carboxylate in a synthesis method of example compound 82.

TABLE 56

| Example | |
|---|---|
| No. | Starting Materials |
| 83 | 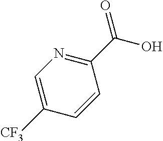 [SMILES: O=C(O)c1ccc(C(F)(F)F)cn1] |
| 102 | 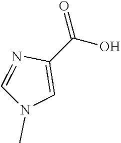 |
| 103 | 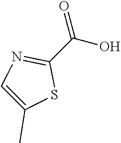 |
| 104 | 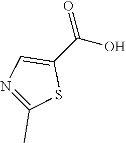 |
| 105 | 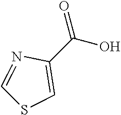 |
| 106 | 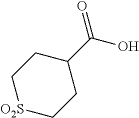 [SMILES: O=C(O)C1CCS(=O)(=O)CC1] |

Analysis data of each of the compounds prepared as described above are shown in the table below.

TABLE 57

| Example | | |
|---|---|---|
| No. | Compound Names | Analysis Data |
| 83 | 4-((7-amino- | ¹H NMR (400 MHz, |
| | 2-(furan-2-yl)- | DMSO-d₆) δ 9.12-8.98 (m, |
| | [1,2,4]triazolo[1,5- | 1H), 8.69-8.00 (m, 3H), |
| | a][1,3,5]triazin- | 7.98-7.77 (m, 2H), 7.15- |
| | 5-yl)-L-prolyl) | 6.98 (m, 1H), 6.75-6.61 |
| | piperazin-1-yl)(5- | (m, 1H), 5.17-4.87 (m, |
| | (trifluoromethyl)pyridin-2- | 1H), 4.01-3.36 (m, 10H), |
| | yl)methanone | 2.39-2.15 (m, 1H), |
| | | 2.06-1.76 (m, 3H); LRMS |
| | | (ES) m/z 557.6 (M⁺ + |
| | | 1). |
| 102 | 4-((7-amino-2- | ¹H NMR (400 MHz, |
| | (furan-2-yl)- | DMSO-d₆) δ 8.69-8.01 (m, |
| | [1,2,4]triazolo[1,5- | 2H), 7.90-7.81 (m, 1H), |
| | a][1,3,5]triazin- | 7.76-7.61 (m, 2H), 7.08- |
| | 5-yl)-L-prolyl) | 7.00 (m, 1H), 6.69-6.61 |
| | piperazin-1-yl)(1- | (m, 1H), 5.15-4.92 (m, |
| | methyl-1H-imidazol-4- | 1H), 4.63-3.51 (m, 12H), |
| | yl)methanone | 3.51-3.39 (m, 1H), 2.39- |
| | | 2.19 (m, 1H), 2.05-1.81 |
| | | (m, 3H); LRMS (ES) |
| | | m/z 492.6 (M⁺ + 1). |
| 103 | 4-((7-amino-2- | ¹H NMR (400 MHz, |
| | (furan-2-yl)- | DMSO-d₆) δ 8.69-7.99 (m, |
| | [1,2,4]triazolo[1,5- | 2H), 7.93-7.81 (m, 1H), |
| | a][1,3,5]triazin- | 7.74 (s, 1H), 7.11-6.99 |
| | 5-yl)-L-prolyl) | (m, 1H), 6.75-6.58 (m, |
| | piperazin-1-yl)(5- | 1H), 5.13-4.91 (m, 1H), |
| | methylthiazol-2- | 4.72-4.13 (m, 2H), |
| | yl)methanone | 3.99-3.39 (m, 8H), 2.55 (s, |
| | | 3H), 2.40-2.17 (m, 1H), |
| | | 2.06-1.79 (m, 3H); |
| | | LRMS (ES) m/z 509.6 (M⁺ + 1). |
| 104 | 4-((7-amino-2- | ¹H NMR (400 MHz, |
| | (furan-2-yl)- | DMSO-d₆) δ 8.18 (s, 2H), 8.07- |
| | [1,2,4]triazolo[1,5- | 7.93 (m, 1H), 7.92-7.81 |
| | a][1,3,5]triazin- | (m, 1H), 7.14-6.97 (m, |
| | 5-yl)-L-prolyl) | 1H), 6.76-6.59 (m, 1H), |
| | piperazin-1-yl)(2- | 5.11-4.91 (m, 1H), 4.09- |
| | methylthiazol-5- | 3.38 (m, 10H), 2.70 (s, |
| | yl)methanone | 3H), 2.38-2.17 (m, 1H), |
| | | 2.09-1.78 (m, 3H); LRMS |
| | | (ES) m/z 509.6 (M⁺ + |
| | | 1). |
| 105 | 4-((7-amino-2- | ¹H NMR (400 MHz, |
| | (furan-2-yl)- | DMSO-d₆) δ 9.21 (s, 1H), 8.74- |
| | [1,2,4]triazolo[1,5- | 7.95 (m, 3H), 7.93-7.82 |
| | a][1,3,5]triazin- | (m, 1H), 7.15-6.97 (m, |
| | 5-yl)-L-prolyl)piperazin-1- | 1H), 6.76-6.61 (m, 1H), |
| | yl)(thiazol-4- | 5.18-4.87 (m, 1H), 4.09- |
| | yl)methanone | 3.38 (m, 10H), 2.31 (d, |
| | | J = 22.9 Hz, 1H), 2.06- |
| | | 1.77 (m, 3H); LRMS |
| | | (ES) m/z 495.5 (M⁺ + 1). |
| 106 | 4-((7-amino- | ¹H NMR (400 MHz, |
| | 2-(furan-2-yl)- | DMSO-d₆) δ 8.70-8.02 (m, |
| | [1,2,4]triazolo[1,5- | 2H), 7.90-7.82 (m, 1H), |
| | a][1,3,5]triazin- | 7.08-6.98 (m, 1H), 6.70- |
| | 5-yl)-L-prolyl) | 6.63 (m, 1H), 5.14-4.92 |
| | piperazin-1-yl)(1,1- | (m, 1H), 3.95-3.38 |
| | dioxidotetrahydro- | (m, 10H), 3.29-3.17 |
| | 2H-thiopyran- | (m, 2H), 3.17-2.98 (m, 3H), |
| | 4-yl)methanone | 2.39-2.18 (m, 1H), |
| | | 2.16-1.98 (m, 4H), 1.98- |
| | | 1.79 (m, 3H); LRMS (ES) |
| | | m/z 544.6 (M⁺ + 1). |

### Example 53: Synthesis of Compound 53, 4-(2-(4-((7-amino-2-(furan-2-yl)-[1,2,4]triazolo[1,5-a][1,3,5]triazine-5-yl)-L-prolyl)piperazin-1-yl)ethyl)morpholine

### [Step 1] Synthesis of (7-amino-2-(furan-2-yl)-[1,2,4]triazolo[1,5-a][1,3,5]triazin-5-yl)-L-proline

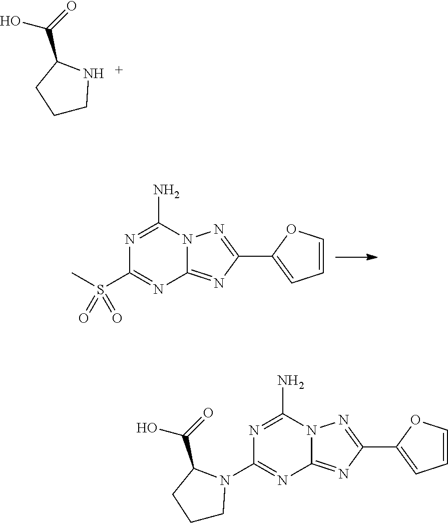

L-Proline (1.151 g, 10.000 mmol), 2-(furan-2-yl)-5-(methylsulfonyl)-[1,2,4]triazolo[1,5-a][1,3,5]triazin-7-amine (2.803 g, 10.000 mmol) and triethylamine (2.788 mL, 20.000 mmol) were dissolved in N,N-dimethylformamide (20 mL) at room temperature, after which the resulting solution was stirred at the same temperature for 18 hours. An obtained product was used without an additional purification process (title compound, 3.151 g, 99.9%, light brown oil).

### [Step 2] Synthesis of 4-(2-(4-((7-amino-2-(furan-2-yl)-[1,2,4]triazolo[1,5-a][1,3,5]triazine-5-yl)-L-prolyl)piperazin-1-yl)ethyl)morpholine

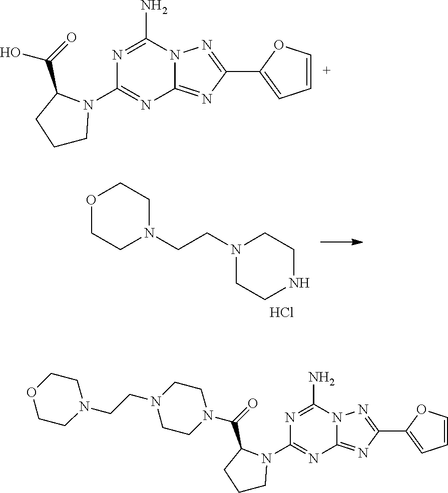 [SMILES: Nc1nc(N2CCC[C@H]2C(=O)O)nc2nc(-c3ccco3)nn12 ; C1CN(CCN2CCOCC2)CCN1 ; Cl ; Nc1nc(N2CCC[C@H]2C(=O)N2CCN(CCN3CCOCC3)CC2)nc2nc(-c3ccco3)nn12]

(7-Amino-2-(furan-2-yl)-[1,2,4]triazolo[1,5-a][1,3,5]triazin-5-yl)-L-proline (0.315 g, 1.000 mmol) prepared in step 1, 4-(2-(piperazin-1-yl)ethyl)morpholine hydrochloride (0.354 g, 1.500 mmol), 2,4,6-tripropyl-1,3,5,2,4,6-trioxatriphosphinane 2,4,6-trioxide (T3P, 50.00% solution in DMF, 1.908 mL, 3.000 mmol) and triethylamine (0.279 mL, 2.000 mmol) were dissolved in N,N-dimethylformamide (5 mL) at room temperature, after which the resulting solution was stirred at the same temperature for 18 hours. Water was poured into the reaction mixture, and then an organic layer was extracted with dichloromethane, filtered via a plastic filter to remove a solid residue and an aqueous solution layer therefrom, and concentrated under reduced pressure. The resulting concentrate was purified via column chromatography (SiO₂, 12 g cartridge; methanol/dichloromethane=0 to 10%) and concentrated to obtain a title compound (0.010 g, 2.0%) as a white solid form.

¹H NMR (400 MHz, DMSO-d₆) δ 8.61-8.00 (m, 2H), 7.92-7.80 (m, 1H), 7.11-6.98 (m, 1H), 6.74-6.60 (m, 1H), 5.09-4.90 (m, 1H), 3.76-3.46 (m, 10H), 2.79-2.63 (m, 1H), 2.49-2.36 (m, 9H), 2.35-2.13 (m, 3H), 1.99-1.79 (m, 3H); LRMS (ES) m/z 497.5 (M⁺+1).

### Example 54: Synthesis of Compound 54

Example compound 54 was synthesized through substantially the same synthesis method as a synthesis method of example compound 53 except for using 1-cyclohexylpiperazine instead of 4-(2-(piperazin-1-yl)ethyl)morpholine hydrochloride.

¹H NMR (400 MHz, DMSO-d₆) δ 8.64-8.01 (m, 2H), 7.92-7.82 (m, 1H), 7.11-6.98 (m, 1H), 6.78-6.60 (m, 1H), 5.08-4.91 (m, 1H), 3.77-3.44 (m, 5H), 3.30-3.08 (m, 1H), 2.84-2.64 (m, 1H), 2.45-2.17 (m, 4H), 2.04-1.66 (m, 7H), 1.59 (d, J=12.4 Hz, 1H), 1.35-1.00 (m, 6H); LRMS (ES) m/z 466.5 (M⁺+1).

### Protocol for Measuring and Analyzing the Activity of Compound of the Present Invention

### Experimental Example 1. Evaluation of A2a Receptor Binding Affinity

The binding affinity of the compounds according to Examples of the present invention to the human adenosine A2a receptor was evaluated by entrusting SB drug discovery in the UK.

A radioligand binding test was conducted by using [3H]-NECA (5′-N-[adenine-2,8-3H]-ethylcarboxamidoadenosine) and an adenosine A2a membrane. As the adenosine A2a membrane, a cell membrane prepared from HEK-293 cells transfected with human adenosine A2a receptor was used. The membrane used for the test was prepared by incubating with a radioligand until equilibrium was reached. In order to separate the radioligand-bound membrane, the unbound radioligand was separated by using Packard filtermate Harverster and glass filter plates.

10 μL of test compound dissolved in binding buffer (50 mM Tris, 10 mM MgCl₂, 1 mM EDTA pH 7.4) and 20 μL of [3H]-NECA (final concentration of 37 nM) or reference inhibitor was mixed with 20 μL of A2a membrane in an unbound 96-well plate and incubated at room temperature for one hour. Prior to filtration, a 96-well harvest filter plate was coated with 0.33% polyethylenimine for 30 minutes and then washed with assay buffer. The binding reaction was transferred to the filter plate and washed three times with wash buffer. The dish was then dried, scintillant was added, and radioactivity was counted in a scintillation counter (Topcount NXT, Packard).

The binding affinity IC₅₀ (μM) for the human adenosine A2a receptor obtained according to the above experimental method is shown in the table below.

TABLE 58

| Example No. | IC₅₀(μM) |
|---|---|
| 1 | 0.021 |
| 2 | 0.001 |
| 3 | 0.001 |
| 4 | 0.000458 |
| 5 | 0.00126 |
| 6 | 0.000214 |
| 7 | 0.00672 |
| 8 | 0.00192 |
| 9 | 0.00242 |
| 10 | 0.000357 |
| 11 | 0.0001 |
| 12 | 0.00143 |
| 13 | 0.0001 |
| 14 | 0.000368 |
| 15 | 0.00602 |
| 16 | 0.00195 |
| 17 | 0.000135 |
| 18 | 0.000125 |
| 19 | 0.015 |
| 20 | 0.02 |
| 21 | 0.03 |
| 22 | 0.011 |
| 23 | 0.061 |
| 24 | 0.033 |
| 25 | 0.01 |
| 26 | 0.004 |
| 27 | 0.007 |
| 28 | 0.008 |
| 29 | 0.008 |
| 30 | 0.001 |
| 31 | 0.001 |
| 32 | 0.0005 |
| 37 | 0.012 |
| 40 | 0.004 |
| 41 | 0.013 |
| 42 | 0.011 |
| 43 | 0.011 |
| 44 | 0.01 |
| 45 | 0.019 |
| 46 | 0.006 |
| 47 | 0.008 |
| 48 | 0.013 |
| 49 | 0.003 |
| 50 | 0.009 |
| 51 | 0.013 |
| 52 | 0.016 |
| 53 | 0.008 |
| 54 | 0.006 |
| 55 | 0.016 |
| 56 | 0.008 |
| 57 | 0.007 |
| 58 | 0.015 |
| 59 | 0.007 |
| 60 | 0.018 |
| 61 | 0.003 |
| 62 | 0.006 |
| 63 | 0.001 |
| 64 | 0.014 |
| 65 | 0.01 |
| 66 | 0.007 |
| 67 | 0.003 |
| 68 | 0.009 |
| 69 | 0.068 |
| 70 | 0.004 |
| 71 | 0.01 |
| 72 | 0.001 |
| 73 | 0.011 |
| 74 | 0.013 |
| 75 | 0.009 |
| 76 | 0.002 |
| 77 | 0.0004 |
| 78 | 0.014 |
| 79 | 0.011 |
| 80 | 0.007 |
| 81 | 0.008 |
| 82 | 0.006 |
| 83 | 0.016 |
| 84 | 0.014 |
| 85 | 0.028 |
| 86 | 0.015 |
| 87 | 0.001 |
| 88 | 0.012 |
| 89 | 0.017 |
| 90 | 0.008 |
| 91 | 0.007 |
| 92 | 2.29 |
| 93 | 3.79 |
| 94 | 4.39 |
| 95 | 4.99 |
| 96 | 1.63 |
| 97 | 3.35 |
| 98 | 11.3 |
| 100 | 9.46 |
| 102 | 0.01 |
| 103 | 0.006 |
| 104 | 0.011 |
| 105 | 0.003 |
| 106 | 0.007 |
| 107 | 0.009 |
| 108 | 0.009 |
| 109 | 0.013 |
| 110 | 0.012 |
| 111 | 0.008 |
| 112 | 0.009 |
| 113 | 0.009 |
| 114 | 0.008 |
| 115 | 0.01 |
| 116 | 0.011 |
| 117 | 0.011 |
| 118 | 0.021 |
| 119 | 0.009 |
| 120 | 0.006 |
| 121 | 0.006 |
| 122 | 0.019 |
| 123 | 0.0005 |
| 124 | 0.002 |
| 125 | 0.009 |
| 126 | 0.009 |
| 127 | 0.003 |
| 128 | 0.003 |
| 129 | 0.01 |
| 130 | 0.002 |
| 131 | 0.004 |
| 132 | 0.011 |
| 133 | 0.011 |
| 134 | 0.086 |
| 135 | 0.024 |
| 136 | 0.004 |
| 137 | 0.048 |
| 138 | 0.002 |
| 139 | 0.028 |
| 140 | 0.01 |
| 141 | 0.015 |
| 142 | 0.013 |
| 143 | 0.02 |
| 144 | 0.014 |
| 145 | 0.013 |
| 146 | 0.018 |
| 147 | 0.003 |
| 148 | 0.008 |
| 149 | 0.0005 |
| 150 | 0.0001 |
| 151 | 0.013 |
| 152 | 0.019 |
| 153 | 0.012 |
| 154 | 0.01 |
| 155 | 0.011 |
| 156 | 0.012 |
| 157 | 0.006 |
| 158 | 0.012 |
| 159 | 0.009 |
| 160 | 0.004 |
| 161 | 0.009 |
| 162 | 0.008 |
| 163 | 0.0005 |
| 164 | 0.0003 |
| 165 | 0.0006 |
| 166 | 0.00008 |
| 167 | 0.0002 |
| 168 | 0.004 |
| 169 | 0.0001 |
| 170 | 0.001 |
| 171 | 0.003 |
| 172 | 0.013 |
| 173 | 0.016 |
| 174 | 0.018 |
| 175 | 0.012 |
| 176 | 0.016 |
| 177 | 0.02 |
| 178 | 0.01 |
| 179 | 0.005 |
| 180 | 0.007 |
| 181 | 0.011 |
| 182 | 0.008 |
| 183 | 0.002 |
| 184 | 0.012 |
| 185 | 0.003 |
| 186 | 0.009 |
| 187 | 0.01 |
| 188 | 0.01 |
| 189 | 0.009 |
| 190 | 0.008 |
| 191 | 0.006 |
| 192 | 0.017 |
| 193 | 0.01 |
| 194 | 0.012 |
| 195 | 0.003 |
| 196 | 0.008 |
| 197 | 0.003 |
| 198 | 0.008 |
| 199 | 0.002 |
| 200 | 0.001 |
| 201 | 0.005 |
| 202 | 0.007 |
| 203 | 0.006 |
| 204 | 0.013 |
| 205 | 0.007 |
| 206 | 0.001 |
| 207 | 0.011 |
| 208 | 0.003 |
| 209 | 0.001 |
| 210 | 0.001 |
| 211 | 0.015 |
| 212 | 0.012 |
| 213 | 0.007 |
| 214 | 0.005 |
| 215 | 0.008 |
| 216 | 0.018 |
| 217 | 0.003 |
| 218 | 0.01 |
| 219 | 0.003 |
| 220 | 0.015 |
| 221 | 0.01 |
| 222 | 0.008 |
| 223 | 0.007 |
| 224 | 0.006 |
| 225 | 0.002 |
| 226 | 0.007 |
| 227 | 0.009 |
| 228 | 0.002 |
| 229 | 0.012 |
| 230 | 0.006 |
| 231 | 0.005 |
| 232 | 0.04 |
| 233 | 0.014 |
| 234 | 0.012 |
| 235 | 0.01 |
| 236 | 0.002 |
| 237 | 0.008 |
| 238 | 0.003 |
| 239 | 0.006 |
| 240 | 0.0003 |
| 241 | 0.001 |
| 242 | 0.009 |
| 243 | 0.014 |
| 244 | 0.004 |
| 245 | 0.015 |
| 246 | 0.003 |
| 247 | 0.002 |
| 248 | 0.013 |
| 249 | 0.009 |
| 250 | 0.009 |
| 251 | 0.013 |
| 252 | 0.006 |
| 253 | 0.007 |
| 254 | 0.012 |
| 255 | 0.009 |
| 256 | 0.001 |
| 257 | 0.0008 |
| 258 | 0.008 |
| 259 | 0.003 |
| 260 | 0.0009 |
| 261 | 0.0009 |
| 262 | 0.005 |
| 263 | 0.025 |
| 264 | 0.01 |
| 265 | 0.044 |
| 266 | 0.015 |
| 267 | 0.0108 |
| 269 | 0.039 |
| 270 | 0.009 |
| 271 | 0.01 |
| 272 | 0.015 |
| 273 | 0.02 |
| 274 | 0.008 |
| 275 | 0.026 |
| 276 | 0.007 |
| 277 | 0.012 |
| 278 | 0.02 |
| 279 | 0.014 |
| 280 | 0.009 |
| 281 | 0.009 |
| 282 | 0.006 |
| 283 | 0.012 |
| 284 | 0.005 |
| 285 | 0.0002 |
| 286 | 0.002 |
| 287 | 0.003 |
| 288 | 0.003 |
| 289 | 0.009 |
| 290 | 0.002 |
| 291 | 0.005 |
| 292 | 0.021 |
| 293 | 0.003 |
| 294 | 0.004 |
| 295 | 0.001 |
| 296 | 0.0008 |
| 297 | 0.015 |
| 298 | 0.003 |
| 299 | 0.009 |
| 300 | 0.009 |
| 301 | 0.003 |
| 302 | 0.006 |
| 303 | 0.006 |
| 304 | 0.011 |
| 305 | 0.016 |
| 306 | 0.03 |
| 307 | 0.007 |
| 308 | 0.013 |
| 309 | 0.021 |
| 310 | 0.012 |
| 311 | 0.002 |
| 312 | 0.002 |
| 313 | 0.005 |
| 314 | 0.019 |
| 315 | 0.016 |
| 316 | 0.026 |
| 317 | 0.013 |
| 318 | 0.019 |
| 319 | 0.015 |
| 320 | 0.002 |
| 321 | 0.002 |
| 322 | 0.006 |
| 323 | 0.003 |
| 324 | 0.005 |
| 325 | 0.008 |
| 326 | 0.0008 |
| 327 | 0.011 |
| 328 | 0.025 |
| 329 | 0.026 |
| 330 | 0.011 |
| 331 | 0.01 |
| 332 | 0.005 |
| 333 | 0.009 |
| 334 | 0.013 |
| 335 | 0.001 |
| 336 | 0.034 |
| 337 | 0.009 |
| 338 | 0.014 |
| 339 | 0.018 |
| 340 | 0.074 |
| 341 | 0.028 |
| 342 | 0.008 |
| 343 | 0.009 |
| 344 | 0.016 |
| 345 | 0.013 |
| 346 | 0.008 |
| 347 | 0.017 |
| 348 | 0.005 |
| 349 | 0.0122 |
| 350 | 0.009 |
| 351 | 0.017 |
| 352 | 0.006 |
| 353 | 0.008 |
| 354 | 0.022 |
| 355 | 0.014 |
| 356 | 0.01 |
| 357 | 0.009 |
| 358 | 0.01 |
| 359 | 0.005 |
| 360 | 0.01 |
| 361 | 0.009 |
| 362 | 0.03 |
| 363 | 0.012 |
| 364 | 0.012 |
| 365 | 0.013 |
| 366 | 0.008 |
| 367 | 0.014 |
| 368 | 0.003 |
| 369 | 0.014 |
| 370 | 0.004 |
| 371 | 0.011 |
| 372 | 0.001 |
| 373 | 0.002 |
| 374 | 0.0008 |
| 375 | 0.008 |
| 376 | 0.001 |
| 377 | 0.0001 |
| 378 | 0.0007 |
| 379 | 0.002 |
| 380 | 0.005 |
| 381 | 0.009 |
| 382 | 0.003 |
| 383 | 0.002 |
| 384 | 0.0005 |
| 385 | 0.0008 |
| 386 | 0.008 |
| 387 | 0.001 |
| 388 | 0.009 |
| 389 | 0.007 |
| 390 | 0.008 |
| 391 | 0.007 |
| 392 | 0.009 |
| 393 | 0.013 |
| 394 | 0.015 |
| 395 | 0.018 |
| 396 | 0.029 |
| 397 | 0.012 |
| 398 | 0.02 |
| 399 | 0.005 |
| 400 | 0.014 |
| 401 | 0.011 |
| 402 | 0.01 |
| 403 | 0.009 |
| 404 | 0.018 |
| 405 | 0.071 |
| 406 | 0.017 |

From the above results, it can be confirmed that the compounds according to an exemplary embodiment of the present invention have very good binding affinity to the human adenosine A2a receptor.

### Experimental Example 2. Kinetic Solubility Test

The solubility of the compound of the present invention was measured to evaluate the physical properties. Reagents and plates used in the experiment are shown in the table below.

| | |
|---|---|
| Dimethylsulfoxide | Bioreagent for molecular |
| (DMSO) | biology ≥99.9%_Sigma |
| | Aldrich D8418-1L_Lot #SHBF2128V |
| Buffer solution | 10xPBS western blotting |
| (Phosphate Buffered | and IP_Sigma Aldrich |
| Saline, PBS) | P7059-1L_Lot #SLBM5027V |
| Simulated Gastric | 0.2% (w/v) sodium chloride in 0.7% (v/v) |
| Fluid (Without | hydrochloric acid |
| pepsin, pH 1.0-1.4) | Ricca Chemical Company 7108-16_500 mL |
| Simulated intestinal | SIF powder_FaSSIF/FeSSIF/FaSSGF |
| fluid (Fasted | 5.8 g_Biorelevant_FFF01 |
| state simulating | FaSSIF Buffer Concentrate 215 |
| intestinal fluid | g_ Biorelevant_FASBUF01 |
| (FASSIF), | |
| pH 6.6-7.0)) | |
| Plate | Microplate, 96well, PP, V-bottom |
| | clear_greiner bio-one_651201 |
| | Microplate, 96well, PS, F-bottom crystal- |
| | clear_greiner bio-one_655101 |

Each of the compounds according to an exemplary embodiment of the present invention was dissolved in dimethyl sulfoxide to prepare solutions at various concentrations (500, 370, 250, 125, 62.5, 31.25, 15.62, 7.81, 3.90, 1.95 μM). After taking the solution by 10 uL and mixing with 190 uL of the prepared solution (buffer solution, simulated gastric juice or simulated intestinal fluid), an amount precipitated at each concentration after one hour was measured with a nephelometer (NEPHELOstar (BMG LABTECH)) to evaluate solubility.

The apparatus is a device for measuring solubility using nephelometry. After dissolving a compound at various concentrations in a desired solvent, when a laser is passed through the solution, solubility may be measured based on a size of a scattering duct by insoluble particles.

As a result, it was confirmed that the compounds of the present invention exhibit excellent solubility in neutral conditions and gastric and intestinal fluid conditions in the digestive tract, respectively.

While the present invention has been described in detail above, it is apparent to those skilled in the art that such detailed descriptions are set forth to illustrate exemplary embodiments only, but are not construed to limit the scope of the present invention. Thus, it should be understood that the substantial scope of the present invention is defined by the accompanying claims and equivalents thereto.

1. A compound represented by formula 1, stereoisomers thereof or pharmaceutically acceptable salts thereof: 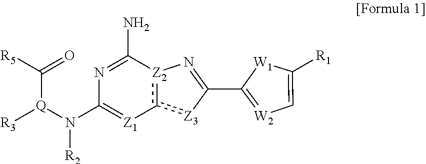
wherein,
W₁ is O or S;
W₂ is N or CH;
Z₁ is CH or N;
Z₂ is C or N;
Z₃ is N, O or S; [symbol]
and [symbol] each independently represent a single bond or a double bond (when [symbol] is a double bond, [symbol] is a single bond, and when [symbol] is a single bond, [symbol] is a double bond);
Q is C—R₄ or N;
R₁ is H or —CH₃;
R₂ is H or C1-C5 alkyl, R₃ is H or —La-Ra, or R₂ and R₃ are linked to form a ring,
in which La is a single bond or C1-C3 alkylene, Ra is C1-C5 alkyl, C3-C6 cycloalkyl, 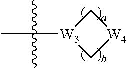
(a and b are each independently 1 or 2, W₃ is CH or N, W₄ is CH₂ or O, in which if W₃ is CH, then W₄ is not CH₂), phenyl or -phenylen-O-benzyl, and if Ra is C1-C5 alkyl or phenyl, then at least one of each H may be substituted with —OH or C1-C5 alkoxy;
a ring formed by linking R₂ and R₃ is a 4- to 6-membered N-containing heterocycloalkyl (in which at least one H of the N-containing heterocycloalkyl may be each independently substituted with C1-C5 alkyl or OH), or a 6- to 8-membered N-containing spiroheterocycloalkyl;
R₄ is H or C1-C5 alkyl;
R₅ is
—NH—(CH₂)_{y}—R_{b} (in which y is any one integer of 1 to 3, and R_{b} is a 5- or 6-membered heterocycloalkyl including any one of O and N); 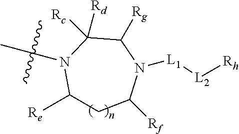
(in which n is 0 or 1, and R_{c}, R_{d}, Rₑ, R_{f} and R_{g} are each independently H or C1-C5 alkyl, but two selected from R_{c}, R_{d}, Rₑ, R_{f} and R_{g} may be linked to form CH₂ or CH₂—CH₂); 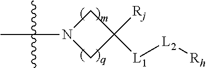
(in which m and q are each independently any one integer of 0 to 3, m and q may not be 0 at the same time, and Rⱼ is H or halogen); 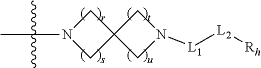
(in which r, s, t and u are each independently 1 or 2); 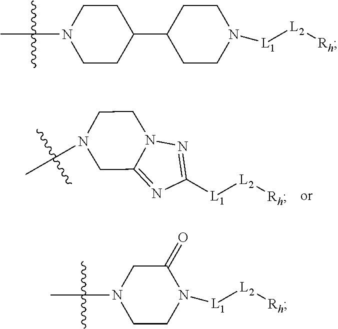
in above R₅,
L₁ is a single bond or C1-C3 alkylene;
L₂ is a single bond, —C(═O)—, —C(═O)NH—, —C(═O)—N(C1-C5 alkyl)-, —C(═O)—NH(C1-C5 alkylene)-, —S(═O)₂— or —S(═O)₂—(C1-C3 alkylene)-;
Rₕ is H, C1-C5 alkyl, C1-C5 alkoxy, C1-C5 haloalkyl, halogen, C3-C6 cycloalkyl, phenoxy, phenyl, —(C1-C5 alkylene)-phenyl, -phenylen-O—(C1-C5 alkyl), -phenylen-C(═O), -phenylen-piperazinyl, 4- to 6-membered heterocycloalkyl including 1 to 3 heteroatoms of at least one selected from N, O and S, 5- to 10-membered heteroaryl including 1 to 3 heteroatoms of at least one selected from N, O, and S, 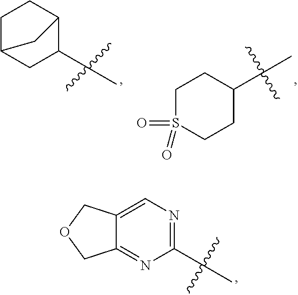 [SMILES: *C1CC2CCC1C2 ; *C1CCS(=O)(=O)CC1 ; *c1ncc2c(n1)COC2]
or —NR₆R₇;
R₆ and R₇ are each independently C1-C5 alkyl or C1-C5 haloalkyl; and
at least one H of Rₕ may be each independently substituted with C1-C5 alkyl, C1-C5 alkoxy, C1-C5 haloalkyl, OH or halogen.

2. The compound represented by formula 1, stereoisomers thereof or pharmaceutically acceptable salts thereof according to claim 1,
wherein in formula 1,
W₁, W₂, Z₁, Z₂, Z₃, Q, R₁, R₂, R₃, R₄, [symbol] and [symbol] are each same as defined in claim 1;
if W₁ is O, then W₂ is CH;
if W₁ is S, then W₂ is N;
R₅ is
—NH—(CH₂)_{y}—R_{b} (in which y is any one integer of 1 to 3, and R_{b} is a 5- or 6-membered heterocycloalkyl including O); 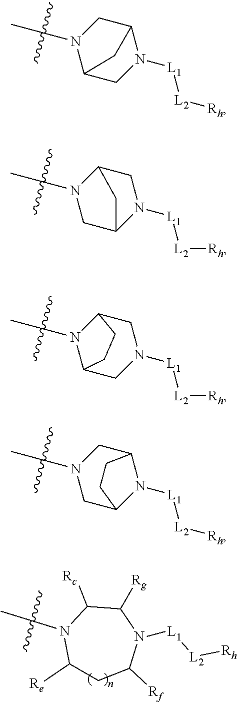
(in which n is 0 or 1, R_{c}, Rₑ, R_{f} and R_{g} are each independently H or C1-C5 alkyl) or 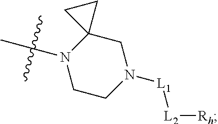 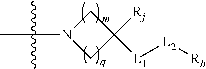
(in which m and q are each independently any one integer of 0 to 3, m and q may not be 0 at the same time, and Rⱼ is H or halogen); 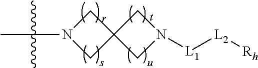
(in which r, s, t and u are each independently 1 or 2); 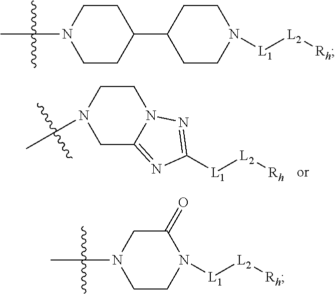
in above R₅, L₁, L₂ and Rₕ are same as defined in claim 1.

3. The compound represented by formula 1, stereoisomers thereof or pharmaceutically acceptable salts thereof according to claim 1,
wherein in formula 1,
W₁, W₂, Z₁, Z₂, Z₃, [symbol] and [symbol] are each same as defined in claim 1;
Q is C—R₄;
R₁ and R₂ are each H;
R₃ is H or -La-Ra (in which La is a single bond or C1-C3 alkylene; Ra is C1-C5 alkyl, C3-C6 cycloalkyl, 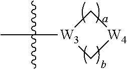
(a and b are each independently 1 or 2, W₃ is CH or N, W₄ is CH₂ or O, in which if W₃ is CH, then W₄ is not CH₂), phenyl or -phenylen-O-benzyl, and if Ra is C1-C5 alkyl or phenyl, at least one of each H may be substituted with —OH or C1-C5 alkoxy;
R₄ is H or C1-C5 alkyl;
R₅ is 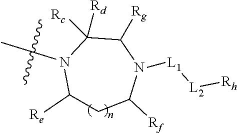
(in which n is 0 or 1, and R_{c}, R_{d}, Rₑ, R_{f} and R_{g} are each independently H or C1-C5 alkyl, but two selected from R_{c}, R_{d}, Rₑ, R_{f} and R_{g} may be linked to form CH₂ or CH₂—CH₂); 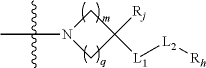
(in which m and q are each independently any one integer of 0 to 3, and Rⱼ is H or halogen); 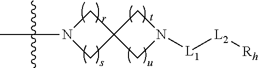
(in which r, s, t and u are each independently 1 or 2); 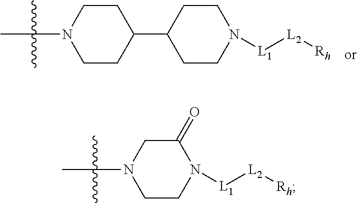
in above R₅,
L₁ is a single bond or C1-C3 alkylene;
L₂ is a single bond, —C(═O)— or —S(═O)₂—;
Rₕ is H, C1-C5 alkyl, C1-C5 alkoxy, C1-C5 haloalkyl, C3-C6 cycloalkyl, phenoxy, phenyl, 5- or 6-membered heterocycloalkyl including 1 to 3 heteroatoms of at least one selected from N and 0, or 5- or 6-membered heteroaryl including 1 to 3 heteroatoms of at least one selected from N and S; and
at least one H of Rₕ may be each independently substituted with C1-C5 alkoxy, C1-C5 haloalkyl, OH or halogen.

4. The compound represented by formula 1, stereoisomers thereof or pharmaceutically acceptable salts thereof according to claim 1,
wherein in formula 1, W₁, W₂, Z₁, Z₂, Z₃, R₁, [symbol] and [symbol] are each same as defined in claim 1;
Q is C—R₄ or N;
R₂ and R₃ are linked with each other to form 4- to 6-membered N-containing heterocycloalkyl (in which at least one H of the N-containing heterocycloalkyl may be each independently substituted with C1-C5 alkyl or OH), or a 6- to 8-membered N-containing spiroheterocycloalkyl;
R₄ is H or C1-C5 alkyl;
R₅ is
—NH—(CH₂)_{y}—R_{b} (in which y is any one integer of 1 to 3, and R_{b} is a 5- or 6-membered heterocycloalkyl including O); 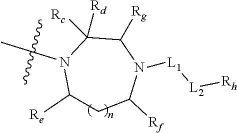
(in which n is 0 or 1, and R_{c}, R_{d}, Rₑ, R_{f} and R_{g} are each independently H or C1-C5 alkyl, but two selected from R_{c}, R_{d}, Rₑ, R_{f} and R_{g} may be linked to form CH₂ or CH₂—CH₂); 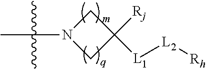
(in which m and q are each independently any one integer of 0 to 3, m and q may not be 0 at the same time, and Rⱼ is H or halogen); 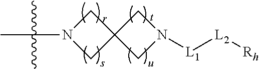
(in which r, s, t and u are each independently 1 or 2); 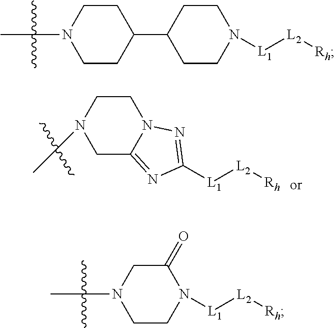
in above R₅,
L₁ is a single bond or C1-C3 alkylene;
L₂ is a single bond, —C(═O)—, —C(═O)NH—, —C(═O)—N(C1-C5 alkyl)-, —C(═O)—NH(C1-C5 alkylene)-, —S(═O)₂— or —S(═O)₂—(C1-C3 alkylene)-;
Rₕ is H, C1-C5 alkyl, C1-C5 alkoxy, C1-C5 haloalkyl, halogen, C3-C6 cycloalkyl, phenoxy, phenyl, —(C1-C3 alkylene)-phenyl, -phenylen-O—(C1-C5 alkyl), -phenylen-C(═O)—, -phenylen-piperazinyl, 4- to 6-membered heterocycloalkyl including 1 to 3 heteroatoms of at least one selected from N, O and S, 5- to 10-membered heteroaryl including 1 to 3 heteroatoms of at least one selected from N, O, and S, 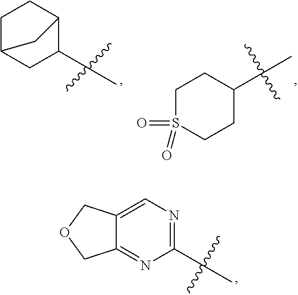 [SMILES: *C1CC2CCC1C2 ; *C1CCS(=O)(=O)CC1 ; *c1ncc2c(n1)COC2]
or —NR₆R₇;
R₆ and R₇ are each independently C1-C5 alkyl or C1-C5 haloalkyl; and
at least one H of Rₕ may be each independently substituted with C1-C5 alkyl, C1-C5 alkoxy, C1-C5 haloalkyl, OH or halogen.

5. A compound, stereoisomers thereof or pharmaceutically acceptable salts thereof, wherein the compound is any one selected from the group consisting of compounds below:

| Example | |
|---|---|
| No. | Compound Strcutre |
| 1 | 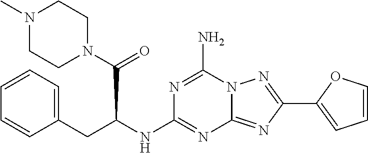 [SMILES: CN1CCN(C(=O)[C@H](Cc2ccccc2)Nc2nc(N)n3nc(-c4ccco4)nc3n2)CC1] |
| 2 | 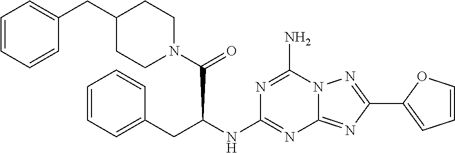 [SMILES: Nc1nc(N[C@@H](Cc2ccccc2)C(=O)N2CCC(Cc3ccccc3)CC2)nc2nc(-c3ccco3)nn12] |
| 3 | 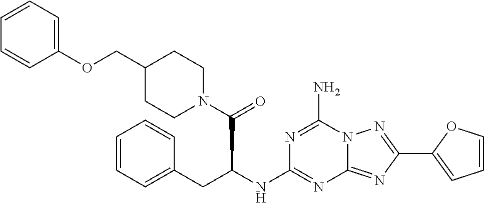 [SMILES: Nc1nc(N[C@@H](Cc2ccccc2)C(=O)N2CCC(COc3ccccc3)CC2)nc2nc(-c3ccco3)nn12] |
| 4 | 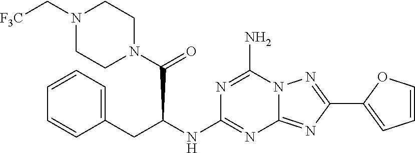 [SMILES: Nc1nc(N[C@@H](Cc2ccccc2)C(=O)N2CCN(CC(F)(F)F)CC2)nc2nc(-c3ccco3)nn12] |
| 5 | 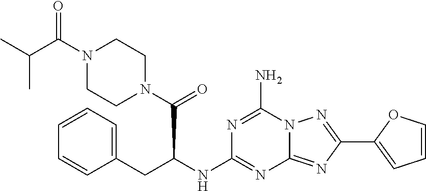 [SMILES: CC(C)C(=O)N1CCN(C(=O)[C@H](Cc2ccccc2)Nc2nc(N)n3nc(-c4ccco4)nc3n2)CC1] |
| 6 | 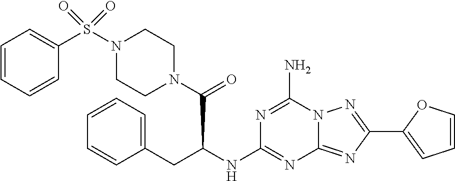 [SMILES: Nc1nc(N[C@@H](Cc2ccccc2)C(=O)N2CCN(S(=O)(=O)c3ccccc3)CC2)nc2nc(-c3ccco3)nn12] |
| 7 | 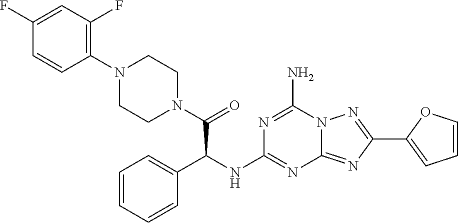 [SMILES: Nc1nc(N[C@H](C(=O)N2CCN(c3ccc(F)cc3F)CC2)c2ccccc2)nc2nc(-c3ccco3)nn12] |
| 8 | 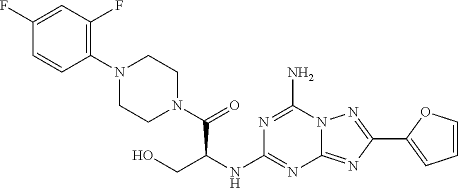 [SMILES: Nc1nc(N[C@@H](CO)C(=O)N2CCN(c3ccc(F)cc3F)CC2)nc2nc(-c3ccco3)nn12] |
| 9 | 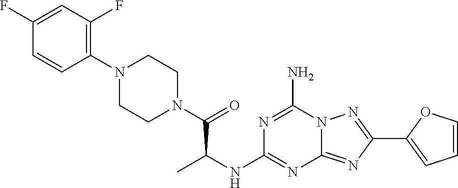 [SMILES: C[C@H](Nc1nc(N)n2nc(-c3ccco3)nc2n1)C(=O)N1CCN(c2ccc(F)cc2F)CC1] |
| 10 | 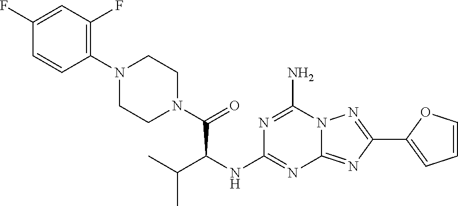 [SMILES: CC(C)[C@H](Nc1nc(N)n2nc(-c3ccco3)nc2n1)C(=O)N1CCN(c2ccc(F)cc2F)CC1] |
| 11 | 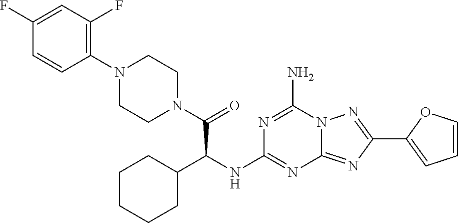 [SMILES: Nc1nc(N[C@H](C(=O)N2CCN(c3ccc(F)cc3F)CC2)C2CCCCC2)nc2nc(-c3ccco3)nn12] |
| 12 | 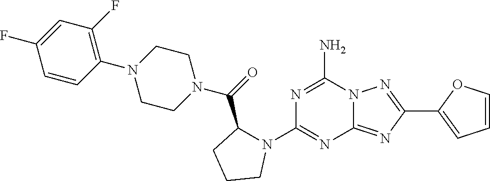 [SMILES: Nc1nc(N2CCC[C@H]2C(=O)N2CCN(c3ccc(F)cc3F)CC2)nc2nc(-c3ccco3)nn12] |
| 13 | 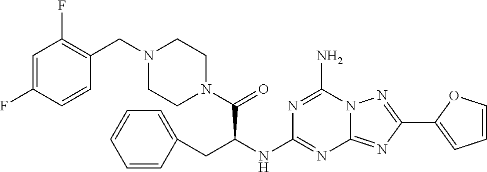 [SMILES: Nc1nc(N[C@@H](Cc2ccccc2)C(=O)N2CCN(Cc3ccc(F)cc3F)CC2)nc2nc(-c3ccco3)nn12] |
| 14 | 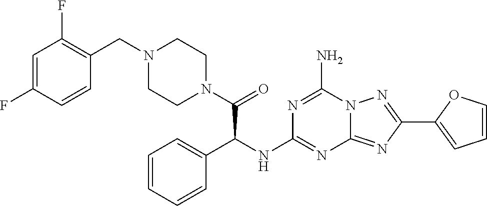 [SMILES: Nc1nc(N[C@H](C(=O)N2CCN(Cc3ccc(F)cc3F)CC2)c2ccccc2)nc2nc(-c3ccco3)nn12] |
| 15 | 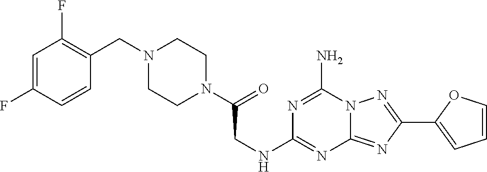 |
| 16 | 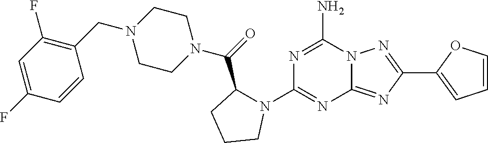 [SMILES: Nc1nc(N2CCC[C@H]2C(=O)N2CCN(Cc3ccc(F)cc3F)CC2)nc2nc(-c3ccco3)nn12] |
| 17 | 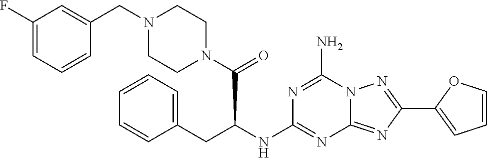 [SMILES: Nc1nc(N[C@@H](Cc2ccccc2)C(=O)N2CCN(Cc3cccc(F)c3)CC2)nc2nc(-c3ccco3)nn12] |
| 18 | 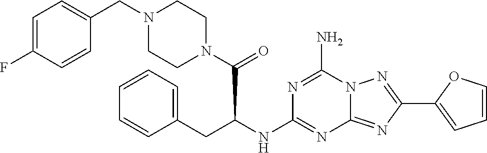 [SMILES: Nc1nc(N[C@@H](Cc2ccccc2)C(=O)N2CCN(Cc3ccc(F)cc3)CC2)nc2nc(-c3ccco3)nn12] |
| 19 | 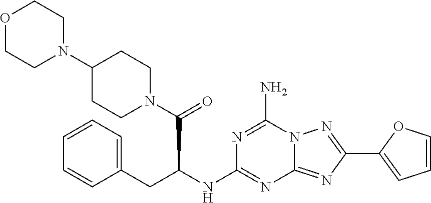 [SMILES: Nc1nc(N[C@@H](Cc2ccccc2)C(=O)N2CCC(N3CCOCC3)CC2)nc2nc(-c3ccco3)nn12] |
| 20 | 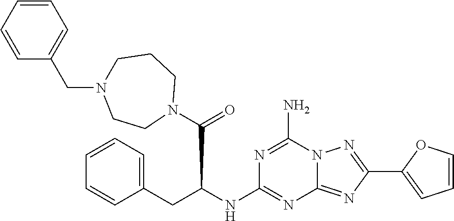 [SMILES: Nc1nc(N[C@@H](Cc2ccccc2)C(=O)N2CCCN(Cc3ccccc3)CC2)nc2nc(-c3ccco3)nn12] |
| 21 | 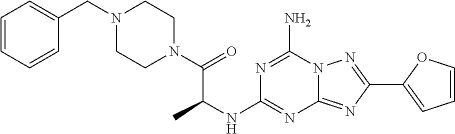 [SMILES: C[C@H](Nc1nc(N)n2nc(-c3ccco3)nc2n1)C(=O)N1CCN(Cc2ccccc2)CC1] |
| 22 | 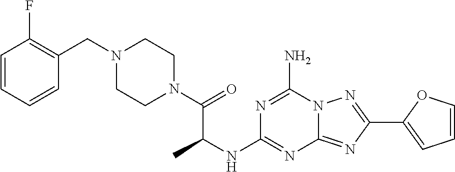 [SMILES: C[C@H](Nc1nc(N)n2nc(-c3ccco3)nc2n1)C(=O)N1CCN(Cc2ccccc2F)CC1] |
| 23 | 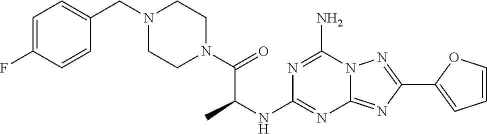 [SMILES: C[C@H](Nc1nc(N)n2nc(-c3ccco3)nc2n1)C(=O)N1CCN(Cc2ccc(F)cc2)CC1] |
| 24 | 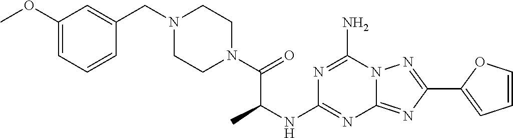 [SMILES: COc1cccc(CN2CCN(C(=O)[C@H](C)Nc3nc(N)n4nc(-c5ccco5)nc4n3)CC2)c1] |
| 25 | 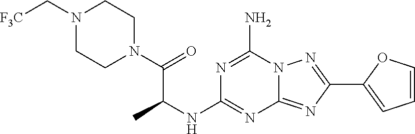 [SMILES: C[C@H](Nc1nc(N)n2nc(-c3ccco3)nc2n1)C(=O)N1CCN(CC(F)(F)F)CC1] |
| 26 | 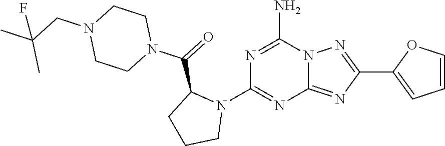 [SMILES: CC(C)(F)CN1CCN(C(=O)[C@@H]2CCCN2c2nc(N)n3nc(-c4ccco4)nc3n2)CC1] |
| 27 | 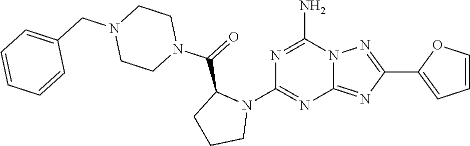 [SMILES: Nc1nc(N2CCC[C@H]2C(=O)N2CCN(Cc3ccccc3)CC2)nc2nc(-c3ccco3)nn12] |
| 28 | 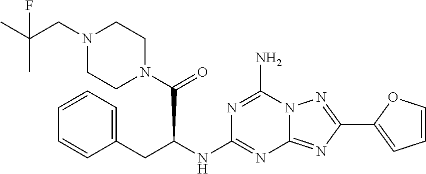 [SMILES: CC(C)(F)CN1CCN(C(=O)[C@H](Cc2ccccc2)Nc2nc(N)n3nc(-c4ccco4)nc3n2)CC1] |
| 29 | 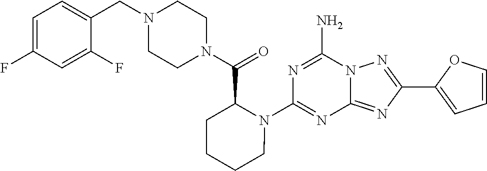 [SMILES: Nc1nc(N2CCCC[C@H]2C(=O)N2CCN(Cc3ccc(F)cc3F)CC2)nc2nc(-c3ccco3)nn12] |
| 30 | 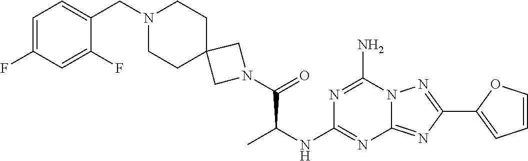 [SMILES: C[C@H](Nc1nc(N)n2nc(-c3ccco3)nc2n1)C(=O)N1CC2(CCN(Cc3ccc(F)cc3F)CC2)C1] |
| 31 | 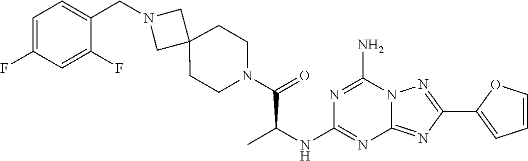 [SMILES: C[C@H](Nc1nc(N)n2nc(-c3ccco3)nc2n1)C(=O)N1CCC2(CC1)CN(Cc1ccc(F)cc1F)C2] |
| 32 | 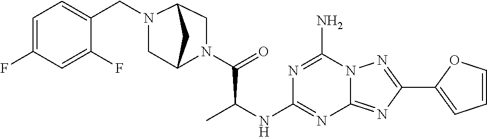 [SMILES: C[C@H](Nc1nc(N)n2nc(-c3ccco3)nc2n1)C(=O)N1C[C@@H]2C[C@H]1CN2Cc1ccc(F)cc1F] |
| 37 | 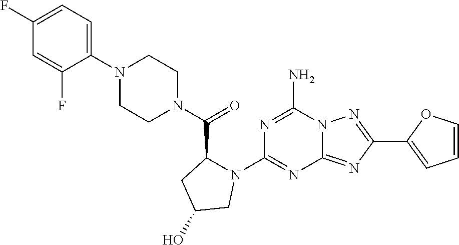 [SMILES: Nc1nc(N2C[C@H](O)C[C@H]2C(=O)N2CCN(c3ccc(F)cc3F)CC2)nc2nc(-c3ccco3)nn12] |
| 40 | 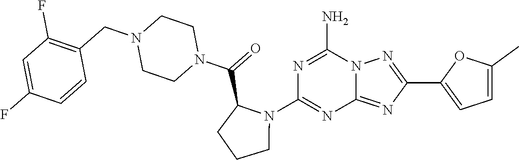 [SMILES: Cc1ccc(-c2nc3nc(N4CCC[C@H]4C(=O)N4CCN(Cc5ccc(F)cc5F)CC4)nc(N)n3n2)o1] |
| 41 | 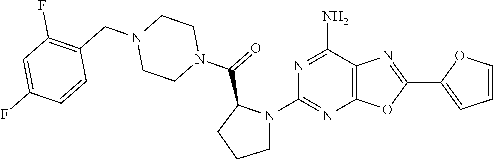 [SMILES: Nc1nc(N2CCC[C@H]2C(=O)N2CCN(Cc3ccc(F)cc3F)CC2)nc2oc(-c3ccco3)nc12] |
| 42 | 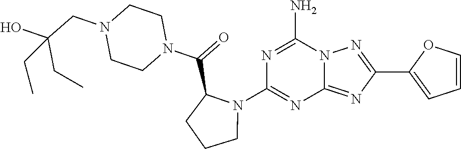 [SMILES: CCC(O)(CC)CN1CCN(C(=O)[C@@H]2CCCN2c2nc(N)n3nc(-c4ccco4)nc3n2)CC1] |
| 43 | 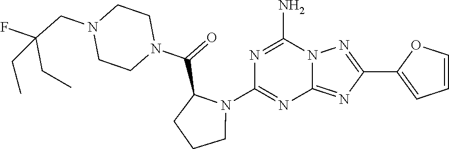 [SMILES: CCC(F)(CC)CN1CCN(C(=O)[C@@H]2CCCN2c2nc(N)n3nc(-c4ccco4)nc3n2)CC1] |
| 44 | 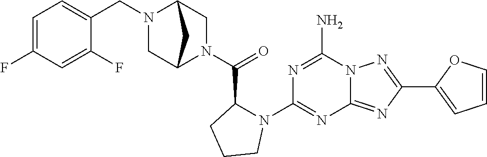 [SMILES: Nc1nc(N2CCC[C@H]2C(=O)N2C[C@@H]3C[C@H]2CN3Cc2ccc(F)cc2F)nc2nc(-c3ccco3)nn12] |
| 45 | 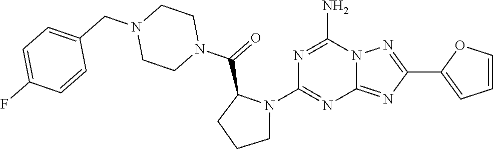 [SMILES: Nc1nc(N2CCC[C@H]2C(=O)N2CCN(Cc3ccc(F)cc3)CC2)nc2nc(-c3ccco3)nn12] |
| 46 | 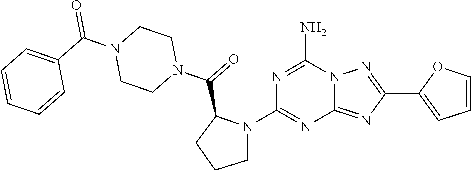 [SMILES: Nc1nc(N2CCC[C@H]2C(=O)N2CCN(C(=O)c3ccccc3)CC2)nc2nc(-c3ccco3)nn12] |
| 47 | 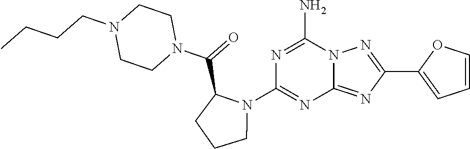 [SMILES: CCCCN1CCN(C(=O)[C@@H]2CCCN2c2nc(N)n3nc(-c4ccco4)nc3n2)CC1] |
| 48 | 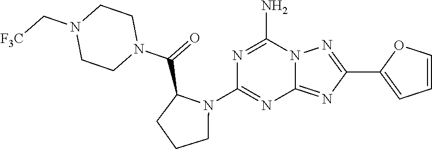 [SMILES: Nc1nc(N2CCC[C@H]2C(=O)N2CCN(CC(F)(F)F)CC2)nc2nc(-c3ccco3)nn12] |
| 49 | 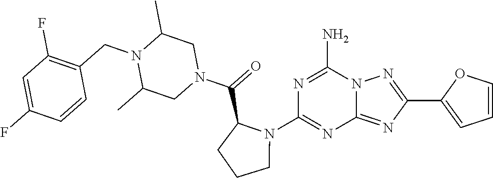 [SMILES: CC1CN(C(=O)[C@@H]2CCCN2c2nc(N)n3nc(-c4ccco4)nc3n2)CC(C)N1Cc1ccc(F)cc1F] |
| 50 | 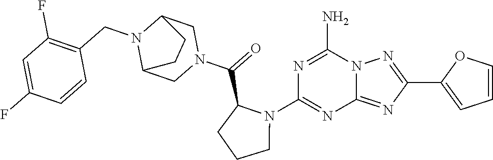 [SMILES: Nc1nc(N2CCC[C@H]2C(=O)N2CC3CCC(C2)N3Cc2ccc(F)cc2F)nc2nc(-c3ccco3)nn12] |
| 51 | 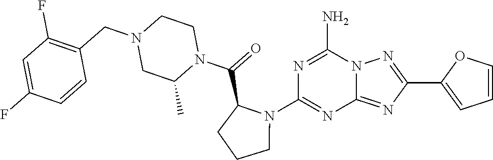 [SMILES: C[C@@H]1CN(Cc2ccc(F)cc2F)CCN1C(=O)[C@@H]1CCCN1c1nc(N)n2nc(-c3ccco3)nc2n1] |
| 52 | 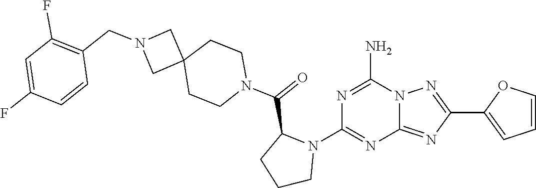 [SMILES: Nc1nc(N2CCC[C@H]2C(=O)N2CCC3(CC2)CN(Cc2ccc(F)cc2F)C3)nc2nc(-c3ccco3)nn12] |
| 53 | 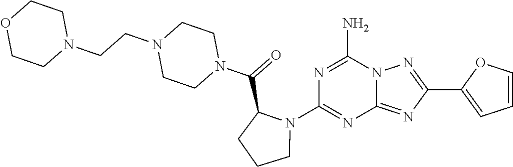 [SMILES: Nc1nc(N2CCC[C@H]2C(=O)N2CCN(CCN3CCOCC3)CC2)nc2nc(-c3ccco3)nn12] |
| 54 | 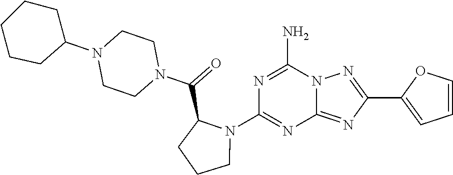 [SMILES: Nc1nc(N2CCC[C@H]2C(=O)N2CCN(C3CCCCC3)CC2)nc2nc(-c3ccco3)nn12] |
| 55 | 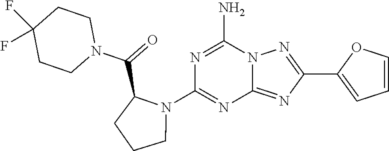 [SMILES: Nc1nc(N2CCC[C@H]2C(=O)N2CCC(F)(F)CC2)nc2nc(-c3ccco3)nn12] |
| 56 | 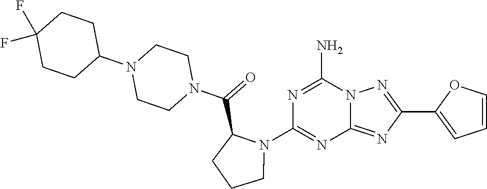 [SMILES: Nc1nc(N2CCC[C@H]2C(=O)N2CCN(C3CCC(F)(F)CC3)CC2)nc2nc(-c3ccco3)nn12] |
| 57 | 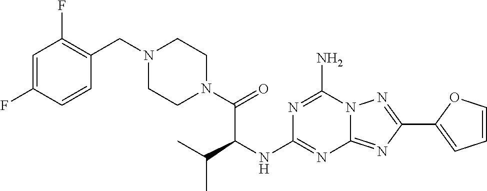 [SMILES: CC(C)[C@H](Nc1nc(N)n2nc(-c3ccco3)nc2n1)C(=O)N1CCN(Cc2ccc(F)cc2F)CC1] |
| 58 | 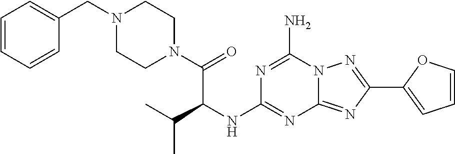 [SMILES: CC(C)[C@H](Nc1nc(N)n2nc(-c3ccco3)nc2n1)C(=O)N1CCN(Cc2ccccc2)CC1] |
| 59 | 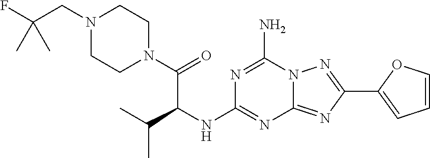 [SMILES: CC(C)[C@H](Nc1nc(N)n2nc(-c3ccco3)nc2n1)C(=O)N1CCN(CC(C)(C)F)CC1] |
| 60 | 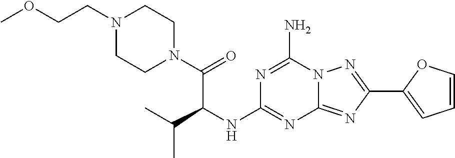 [SMILES: COCCN1CCN(C(=O)[C@@H](Nc2nc(N)n3nc(-c4ccco4)nc3n2)C(C)C)CC1] |
| 61 | 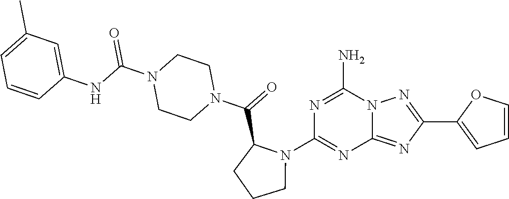 [SMILES: Cc1cccc(NC(=O)N2CCN(C(=O)[C@@H]3CCCN3c3nc(N)n4nc(-c5ccco5)nc4n3)CC2)c1] |
| 62 | 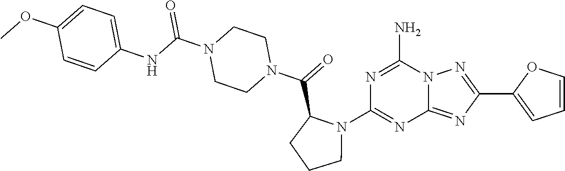 [SMILES: COc1ccc(NC(=O)N2CCN(C(=O)[C@@H]3CCCN3c3nc(N)n4nc(-c5ccco5)nc4n3)CC2)cc1] |
| 63 | 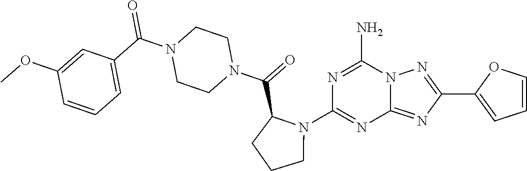 [SMILES: COc1cccc(C(=O)N2CCN(C(=O)[C@@H]3CCCN3c3nc(N)n4nc(-c5ccco5)nc4n3)CC2)c1] |
| 64 | 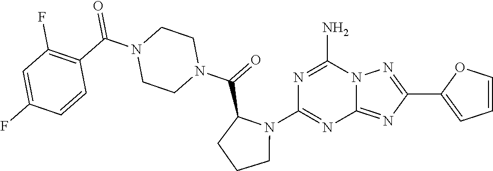 [SMILES: Nc1nc(N2CCC[C@H]2C(=O)N2CCN(C(=O)c3ccc(F)cc3F)CC2)nc2nc(-c3ccco3)nn12] |
| 65 | 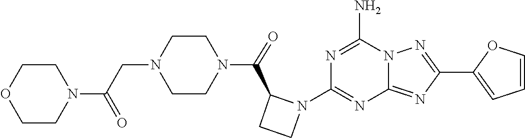 [SMILES: Nc1nc(N2CC[C@H]2C(=O)N2CCN(CC(=O)N3CCOCC3)CC2)nc2nc(-c3ccco3)nn12] |
| 66 | 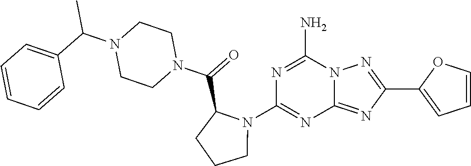 [SMILES: CC(c1ccccc1)N1CCN(C(=O)[C@@H]2CCCN2c2nc(N)n3nc(-c4ccco4)nc3n2)CC1] |
| 67 | 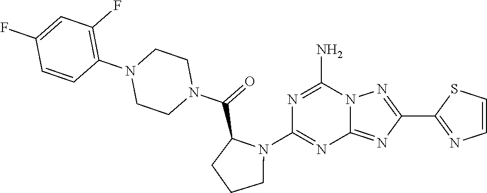 [SMILES: Nc1nc(N2CCC[C@H]2C(=O)N2CCN(c3ccc(F)cc3F)CC2)nc2nc(-c3nccs3)nn12] |
| 68 | 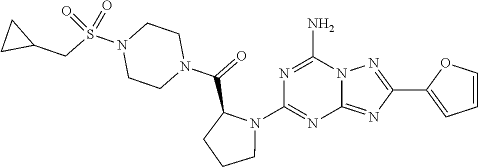 [SMILES: Nc1nc(N2CCC[C@H]2C(=O)N2CCN(S(=O)(=O)CC3CC3)CC2)nc2nc(-c3ccco3)nn12] |
| 69 | 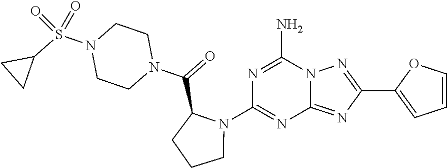 [SMILES: Nc1nc(N2CCC[C@H]2C(=O)N2CCN(S(=O)(=O)C3CC3)CC2)nc2nc(-c3ccco3)nn12] |
| 70 | 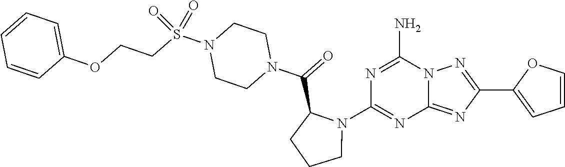 [SMILES: Nc1nc(N2CCC[C@H]2C(=O)N2CCN(S(=O)(=O)CCOc3ccccc3)CC2)nc2nc(-c3ccco3)nn12] |
| 71 | 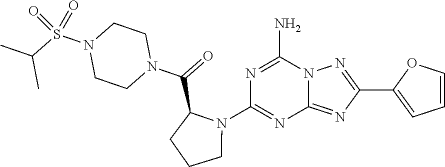 [SMILES: CC(C)S(=O)(=O)N1CCN(C(=O)[C@@H]2CCCN2c2nc(N)n3nc(-c4ccco4)nc3n2)CC1] |
| 72 | 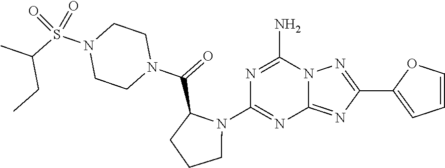 [SMILES: CCC(C)S(=O)(=O)N1CCN(C(=O)[C@@H]2CCCN2c2nc(N)n3nc(-c4ccco4)nc3n2)CC1] |
| 73 | 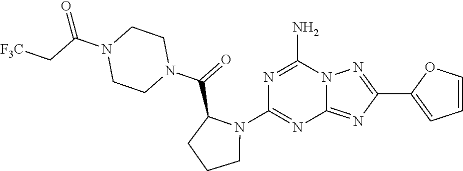 [SMILES: Nc1nc(N2CCC[C@H]2C(=O)N2CCN(C(=O)CC(F)(F)F)CC2)nc2nc(-c3ccco3)nn12] |
| 74 | 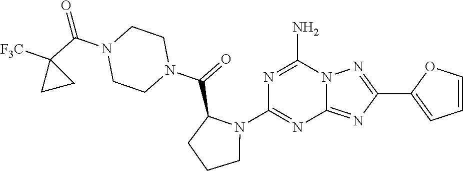 [SMILES: Nc1nc(N2CCC[C@H]2C(=O)N2CCN(C(=O)C3(C(F)(F)F)CC3)CC2)nc2nc(-c3ccco3)nn12] |
| 75 | 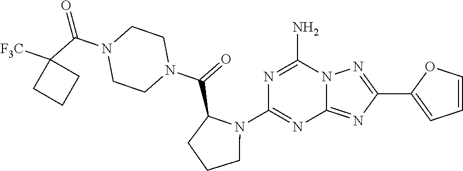 [SMILES: Nc1nc(N2CCC[C@H]2C(=O)N2CCN(C(=O)C3(C(F)(F)F)CCC3)CC2)nc2nc(-c3ccco3)nn12] |
| 76 | 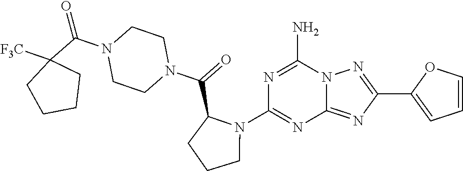 [SMILES: Nc1nc(N2CCC[C@H]2C(=O)N2CCN(C(=O)C3(C(F)(F)F)CCCC3)CC2)nc2nc(-c3ccco3)nn12] |
| 77 | 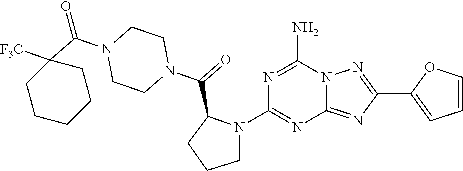 [SMILES: Nc1nc(N2CCC[C@H]2C(=O)N2CCN(C(=O)C3(C(F)(F)F)CCCCC3)CC2)nc2nc(-c3ccco3)nn12] |
| 78 | 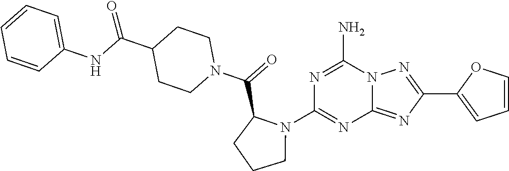 [SMILES: Nc1nc(N2CCC[C@H]2C(=O)N2CCC(C(=O)Nc3ccccc3)CC2)nc2nc(-c3ccco3)nn12] |
| 79 | 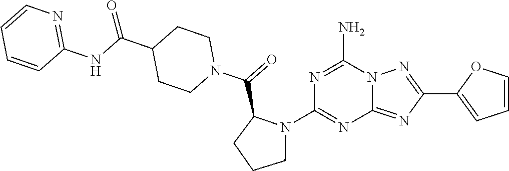 [SMILES: Nc1nc(N2CCC[C@H]2C(=O)N2CCC(C(=O)Nc3ccccn3)CC2)nc2nc(-c3ccco3)nn12] |
| 80 | 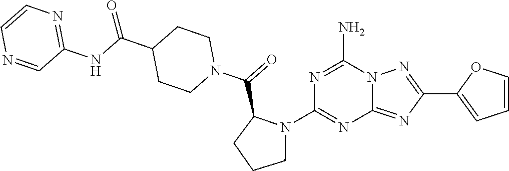 [SMILES: Nc1nc(N2CCC[C@H]2C(=O)N2CCC(C(=O)Nc3cnccn3)CC2)nc2nc(-c3ccco3)nn12] |
| 81 | 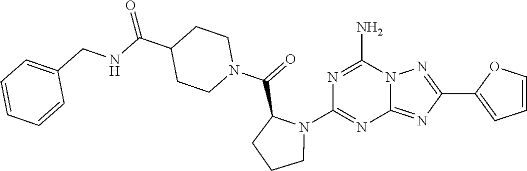 [SMILES: Nc1nc(N2CCC[C@H]2C(=O)N2CCC(C(=O)NCc3ccccc3)CC2)nc2nc(-c3ccco3)nn12] |
| 82 | 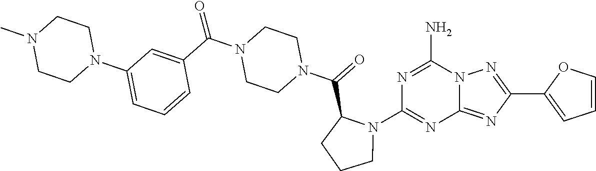 [SMILES: CN1CCN(c2cccc(C(=O)N3CCN(C(=O)[C@@H]4CCCN4c4nc(N)n5nc(-c6ccco6)nc5n4)CC3)c2)CC1] |
| 83 | 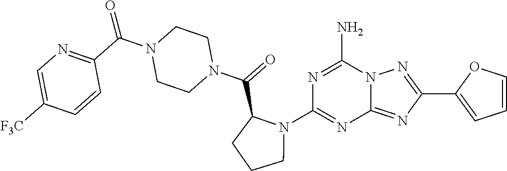 [SMILES: Nc1nc(N2CCC[C@H]2C(=O)N2CCN(C(=O)c3ccc(C(F)(F)F)cn3)CC2)nc2nc(-c3ccco3)nn12] |
| 84 | 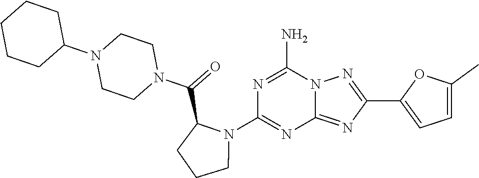 [SMILES: Cc1ccc(-c2nc3nc(N4CCC[C@H]4C(=O)N4CCN(C5CCCCC5)CC4)nc(N)n3n2)o1] |
| 85 | 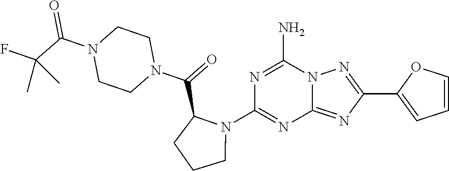 [SMILES: CC(C)(F)C(=O)N1CCN(C(=O)[C@@H]2CCCN2c2nc(N)n3nc(-c4ccco4)nc3n2)CC1] |
| 86 | 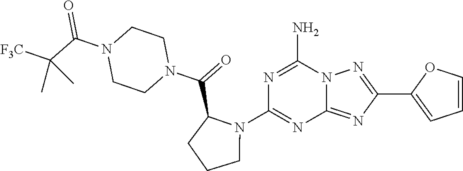 [SMILES: CC(C)(C(=O)N1CCN(C(=O)[C@@H]2CCCN2c2nc(N)n3nc(-c4ccco4)nc3n2)CC1)C(F)(F)F] |
| 87 | 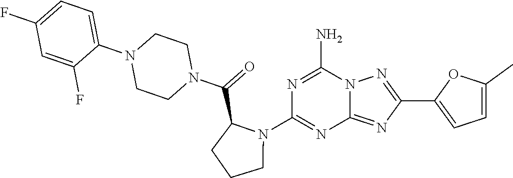 [SMILES: Cc1ccc(-c2nc3nc(N4CCC[C@H]4C(=O)N4CCN(c5ccc(F)cc5F)CC4)nc(N)n3n2)o1] |
| 88 | 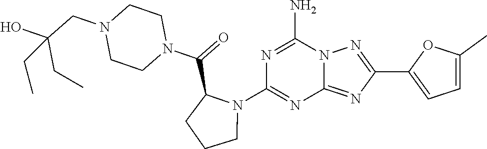 [SMILES: CCC(O)(CC)CN1CCN(C(=O)[C@@H]2CCCN2c2nc(N)n3nc(-c4ccc(C)o4)nc3n2)CC1] |
| 89 | 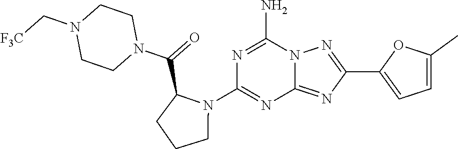 [SMILES: Cc1ccc(-c2nc3nc(N4CCC[C@H]4C(=O)N4CCN(CC(F)(F)F)CC4)nc(N)n3n2)o1] |
| 90 | 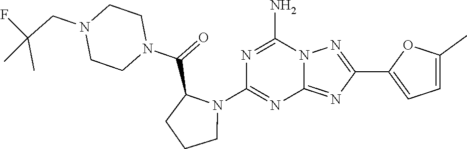 [SMILES: Cc1ccc(-c2nc3nc(N4CCC[C@H]4C(=O)N4CCN(CC(C)(C)F)CC4)nc(N)n3n2)o1] |
| 91 | 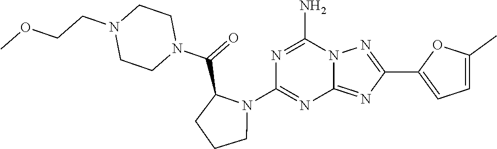 |
| 92 | 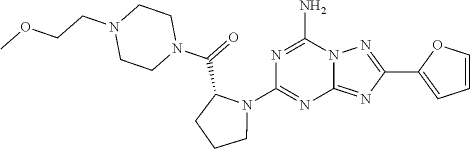 [SMILES: COCCN1CCN(C(=O)[C@H]2CCCN2c2nc(N)n3nc(-c4ccco4)nc3n2)CC1] |
| 93 | 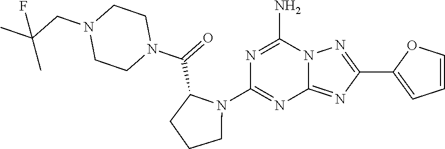 [SMILES: CC(C)(F)CN1CCN(C(=O)[C@H]2CCCN2c2nc(N)n3nc(-c4ccco4)nc3n2)CC1] |
| 94 | 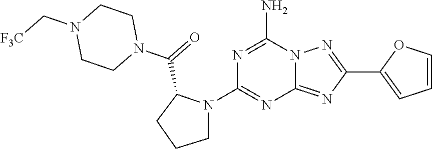 [SMILES: Nc1nc(N2CCC[C@@H]2C(=O)N2CCN(CC(F)(F)F)CC2)nc2nc(-c3ccco3)nn12] |
| 95 | 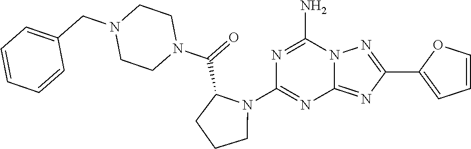 [SMILES: Nc1nc(N2CCC[C@@H]2C(=O)N2CCN(Cc3ccccc3)CC2)nc2nc(-c3ccco3)nn12] |
| 96 | 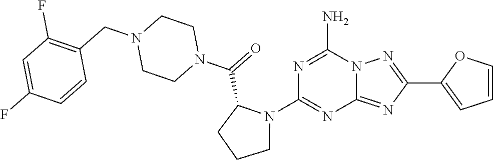 [SMILES: Nc1nc(N2CCC[C@@H]2C(=O)N2CCN(Cc3ccc(F)cc3F)CC2)nc2nc(-c3ccco3)nn12] |
| 97 | 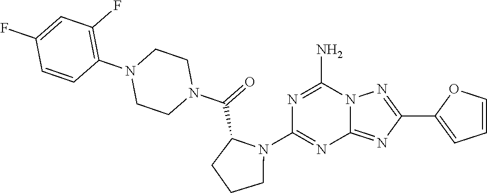 [SMILES: Nc1nc(N2CCC[C@@H]2C(=O)N2CCN(c3ccc(F)cc3F)CC2)nc2nc(-c3ccco3)nn12] |
| 98 | 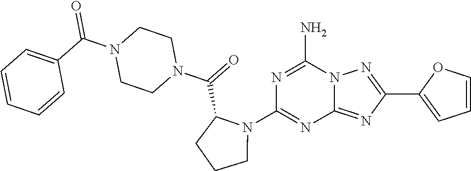 [SMILES: Nc1nc(N2CCC[C@@H]2C(=O)N2CCN(C(=O)c3ccccc3)CC2)nc2nc(-c3ccco3)nn12] |
| 99 | 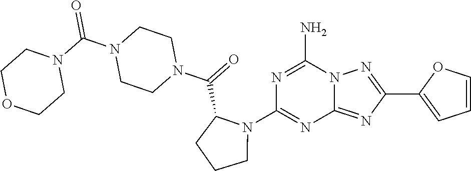 [SMILES: Nc1nc(N2CCC[C@@H]2C(=O)N2CCN(C(=O)N3CCOCC3)CC2)nc2nc(-c3ccco3)nn12] |
| 100 | 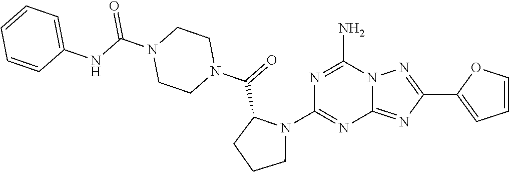 [SMILES: Nc1nc(N2CCC[C@@H]2C(=O)N2CCN(C(=O)Nc3ccccc3)CC2)nc2nc(-c3ccco3)nn12] |
| 101 | 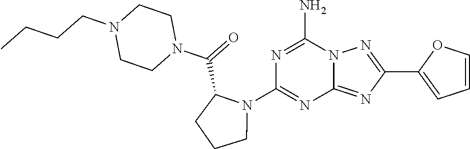 [SMILES: CCCCN1CCN(C(=O)[C@H]2CCCN2c2nc(N)n3nc(-c4ccco4)nc3n2)CC1] |
| 102 | 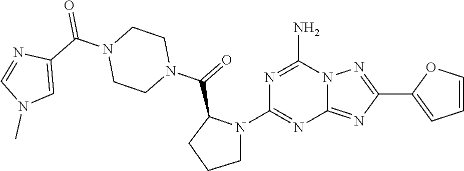 [SMILES: Cn1cnc(C(=O)N2CCN(C(=O)[C@@H]3CCCN3c3nc(N)n4nc(-c5ccco5)nc4n3)CC2)c1] |
| 103 | 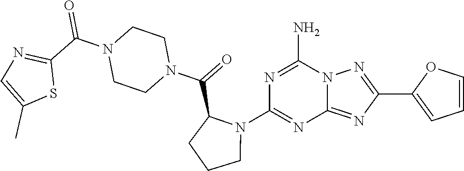 [SMILES: Cc1cnc(C(=O)N2CCN(C(=O)[C@@H]3CCCN3c3nc(N)n4nc(-c5ccco5)nc4n3)CC2)s1] |
| 104 | 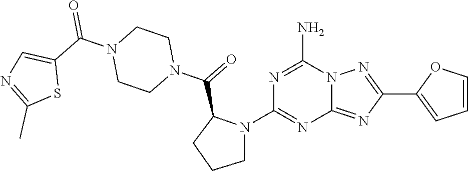 [SMILES: Cc1ncc(C(=O)N2CCN(C(=O)[C@@H]3CCCN3c3nc(N)n4nc(-c5ccco5)nc4n3)CC2)s1] |
| 105 | 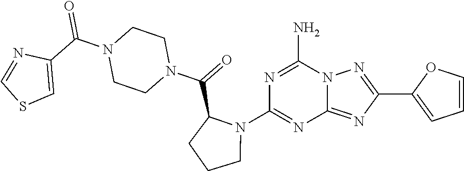 [SMILES: Nc1nc(N2CCC[C@H]2C(=O)N2CCN(C(=O)c3cscn3)CC2)nc2nc(-c3ccco3)nn12] |
| 106 | 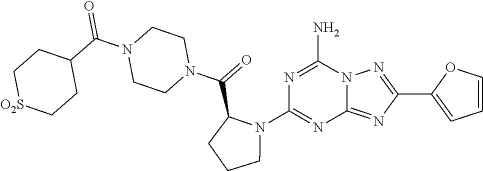 [SMILES: Nc1nc(N2CCC[C@H]2C(=O)N2CCN(C(=O)C3CCS(=O)(=O)CC3)CC2)nc2nc(-c3ccco3)nn12] |
| 107 | 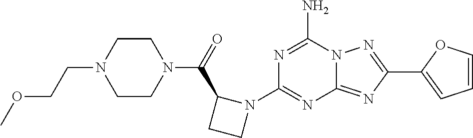 [SMILES: COCCN1CCN(C(=O)[C@@H]2CCN2c2nc(N)n3nc(-c4ccco4)nc3n2)CC1] |
| 108 | 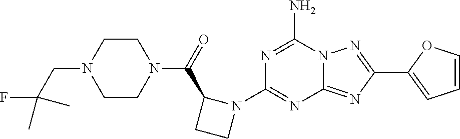 [SMILES: CC(C)(F)CN1CCN(C(=O)[C@@H]2CCN2c2nc(N)n3nc(-c4ccco4)nc3n2)CC1] |
| 109 | 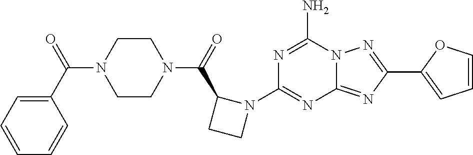 [SMILES: Nc1nc(N2CC[C@H]2C(=O)N2CCN(C(=O)c3ccccc3)CC2)nc2nc(-c3ccco3)nn12] |
| 110 | 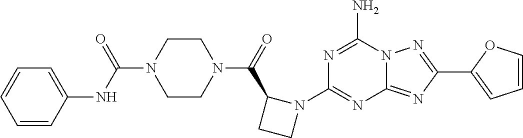 [SMILES: Nc1nc(N2CC[C@H]2C(=O)N2CCN(C(=O)Nc3ccccc3)CC2)nc2nc(-c3ccco3)nn12] |
| 111 | 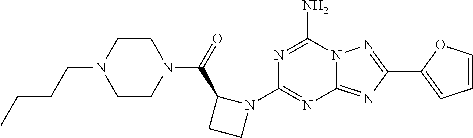 [SMILES: CCCCN1CCN(C(=O)[C@@H]2CCN2c2nc(N)n3nc(-c4ccco4)nc3n2)CC1] |
| 112 | 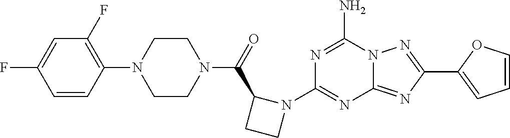 [SMILES: Nc1nc(N2CC[C@H]2C(=O)N2CCN(c3ccc(F)cc3F)CC2)nc2nc(-c3ccco3)nn12] |
| 113 | 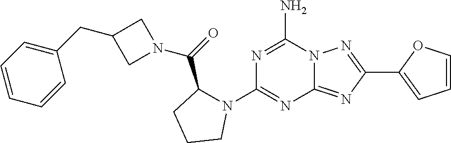 [SMILES: Nc1nc(N2CCC[C@H]2C(=O)N2CC(Cc3ccccc3)C2)nc2nc(-c3ccco3)nn12] |
| 114 | 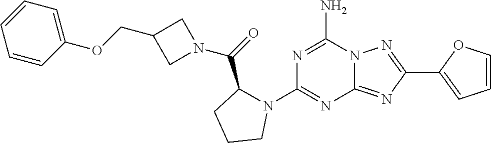 [SMILES: Nc1nc(N2CCC[C@H]2C(=O)N2CC(COc3ccccc3)C2)nc2nc(-c3ccco3)nn12] |
| 115 | 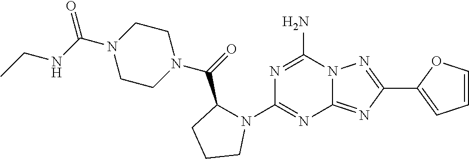 [SMILES: CCNC(=O)N1CCN(C(=O)[C@@H]2CCCN2c2nc(N)n3nc(-c4ccco4)nc3n2)CC1] |
| 116 | 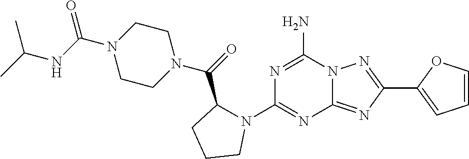 [SMILES: CC(C)NC(=O)N1CCN(C(=O)[C@@H]2CCCN2c2nc(N)n3nc(-c4ccco4)nc3n2)CC1] |
| 117 | 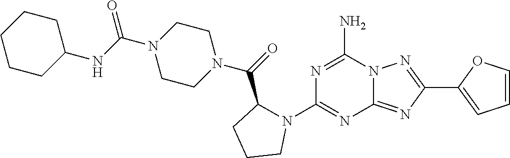 [SMILES: Nc1nc(N2CCC[C@H]2C(=O)N2CCN(C(=O)NC3CCCCC3)CC2)nc2nc(-c3ccco3)nn12] |
| 118 | 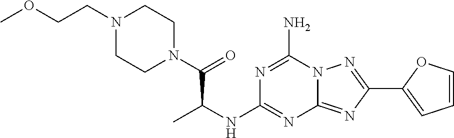 [SMILES: COCCN1CCN(C(=O)[C@H](C)Nc2nc(N)n3nc(-c4ccco4)nc3n2)CC1] |
| 119 | 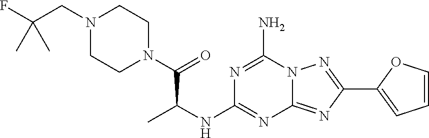 [SMILES: C[C@H](Nc1nc(N)n2nc(-c3ccco3)nc2n1)C(=O)N1CCN(CC(C)(C)F)CC1] |
| 120 | 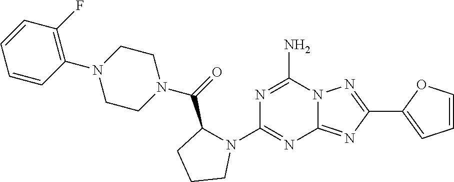 [SMILES: Nc1nc(N2CCC[C@H]2C(=O)N2CCN(c3ccccc3F)CC2)nc2nc(-c3ccco3)nn12] |
| 121 | 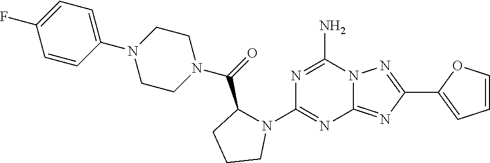 [SMILES: Nc1nc(N2CCC[C@H]2C(=O)N2CCN(c3ccc(F)cc3)CC2)nc2nc(-c3ccco3)nn12] |
| 122 | 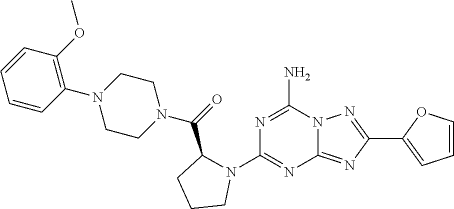 [SMILES: COc1ccccc1N1CCN(C(=O)[C@@H]2CCCN2c2nc(N)n3nc(-c4ccco4)nc3n2)CC1] |
| 123 | 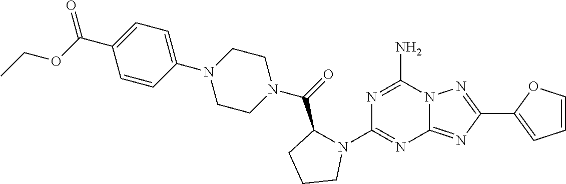 [SMILES: CCOC(=O)c1ccc(N2CCN(C(=O)[C@@H]3CCCN3c3nc(N)n4nc(-c5ccco5)nc4n3)CC2)cc1] |
| 124 | 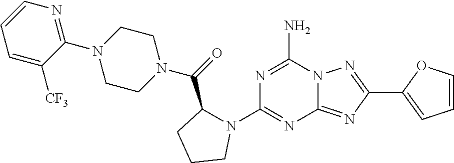 [SMILES: Nc1nc(N2CCC[C@H]2C(=O)N2CCN(c3ncccc3C(F)(F)F)CC2)nc2nc(-c3ccco3)nn12] |
| 125 | 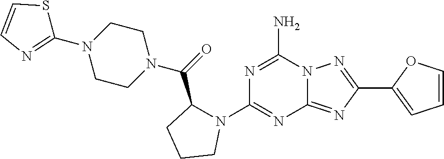 [SMILES: Nc1nc(N2CCC[C@H]2C(=O)N2CCN(c3nccs3)CC2)nc2nc(-c3ccco3)nn12] |
| 126 | 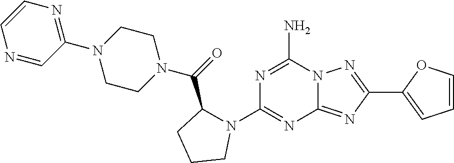 [SMILES: Nc1nc(N2CCC[C@H]2C(=O)N2CCN(c3cnccn3)CC2)nc2nc(-c3ccco3)nn12] |
| 127 | 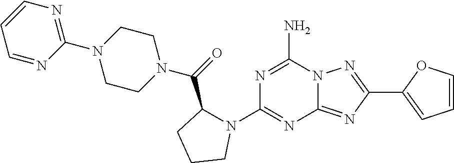 [SMILES: Nc1nc(N2CCC[C@H]2C(=O)N2CCN(c3ncccn3)CC2)nc2nc(-c3ccco3)nn12] |
| 128 | 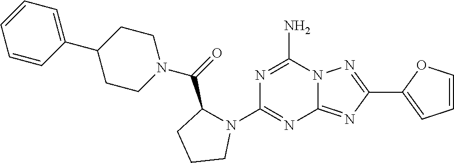 [SMILES: Nc1nc(N2CCC[C@H]2C(=O)N2CCC(c3ccccc3)CC2)nc2nc(-c3ccco3)nn12] |
| 129 | 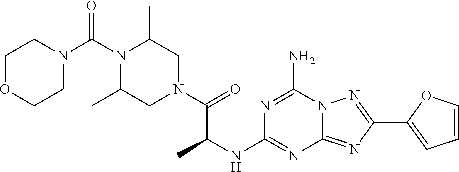 [SMILES: CC1CN(C(=O)[C@H](C)Nc2nc(N)n3nc(-c4ccco4)nc3n2)CC(C)N1C(=O)N1CCOCC1] |
| 130 | 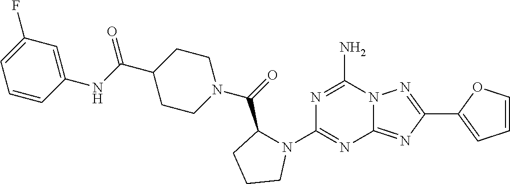 [SMILES: Nc1nc(N2CCC[C@H]2C(=O)N2CCC(C(=O)Nc3cccc(F)c3)CC2)nc2nc(-c3ccco3)nn12] |
| 131 | 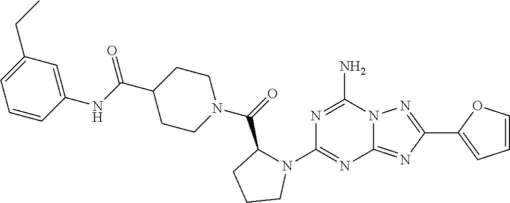 [SMILES: CCc1cccc(NC(=O)C2CCN(C(=O)[C@@H]3CCCN3c3nc(N)n4nc(-c5ccco5)nc4n3)CC2)c1] |
| 132 | 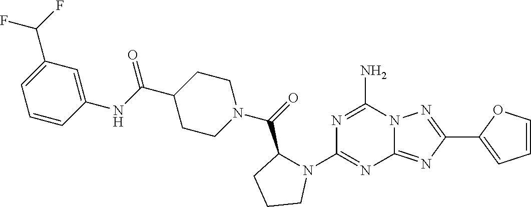 [SMILES: Nc1nc(N2CCC[C@H]2C(=O)N2CCC(C(=O)Nc3cccc(C(F)F)c3)CC2)nc2nc(-c3ccco3)nn12] |
| 133 | 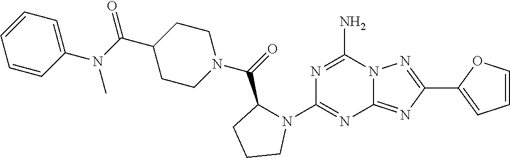 [SMILES: CN(C(=O)C1CCN(C(=O)[C@@H]2CCCN2c2nc(N)n3nc(-c4ccco4)nc3n2)CC1)c1ccccc1] |
| 134 | 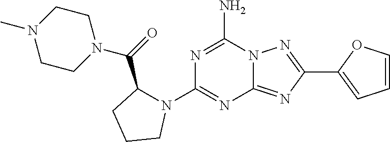 [SMILES: CN1CCN(C(=O)[C@@H]2CCCN2c2nc(N)n3nc(-c4ccco4)nc3n2)CC1] |
| 135 | 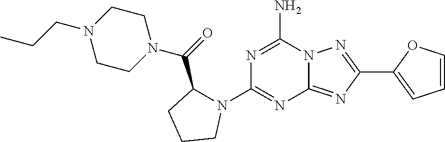 [SMILES: CCCN1CCN(C(=O)[C@@H]2CCCN2c2nc(N)n3nc(-c4ccco4)nc3n2)CC1] |
| 136 | 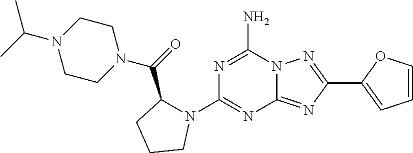 [SMILES: CC(C)N1CCN(C(=O)[C@@H]2CCCN2c2nc(N)n3nc(-c4ccco4)nc3n2)CC1] |
| 137 | 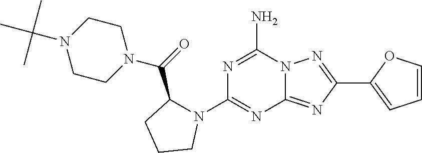 [SMILES: CC(C)(C)N1CCN(C(=O)[C@@H]2CCCN2c2nc(N)n3nc(-c4ccco4)nc3n2)CC1] |
| 138 | 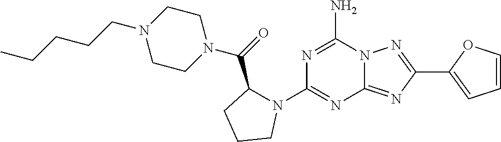 [SMILES: CCCCCN1CCN(C(=O)[C@@H]2CCCN2c2nc(N)n3nc(-c4ccco4)nc3n2)CC1] |
| 139 | 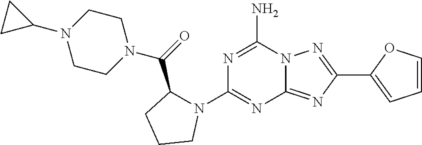 [SMILES: Nc1nc(N2CCC[C@H]2C(=O)N2CCN(C3CC3)CC2)nc2nc(-c3ccco3)nn12] |
| 140 | 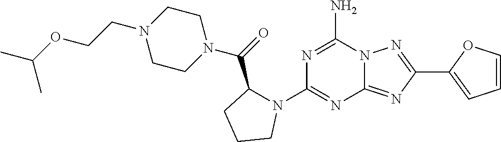 [SMILES: CC(C)OCCN1CCN(C(=O)[C@@H]2CCCN2c2nc(N)n3nc(-c4ccco4)nc3n2)CC1] |
| 141 | 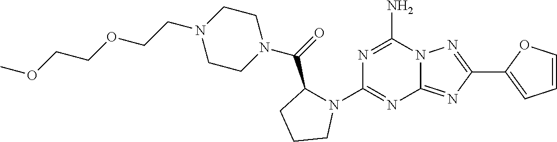 [SMILES: COCCOCCN1CCN(C(=O)[C@@H]2CCCN2c2nc(N)n3nc(-c4ccco4)nc3n2)CC1] |
| 142 | 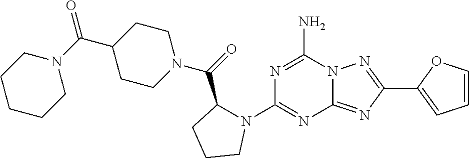 [SMILES: Nc1nc(N2CCC[C@H]2C(=O)N2CCC(C(=O)N3CCCCC3)CC2)nc2nc(-c3ccco3)nn12] |
| 143 | 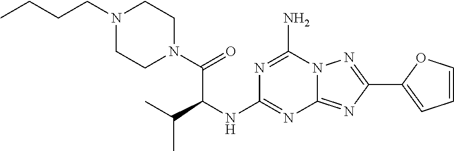 [SMILES: CCCCN1CCN(C(=O)[C@@H](Nc2nc(N)n3nc(-c4ccco4)nc3n2)C(C)C)CC1] |
| 144 | 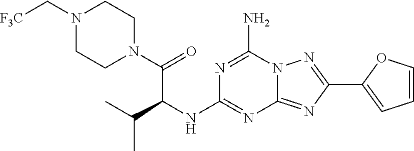 [SMILES: CC(C)[C@H](Nc1nc(N)n2nc(-c3ccco3)nc2n1)C(=O)N1CCN(CC(F)(F)F)CC1] |
| 145 | 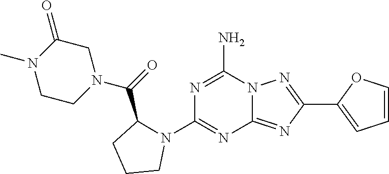 [SMILES: CN1CCN(C(=O)[C@@H]2CCCN2c2nc(N)n3nc(-c4ccco4)nc3n2)CC1=O] |
| 146 | 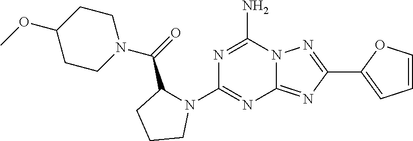 |
| 147 | 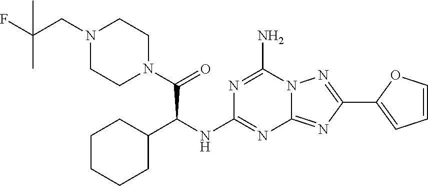 [SMILES: CC(C)(F)CN1CCN(C(=O)[C@@H](Nc2nc(N)n3nc(-c4ccco4)nc3n2)C2CCCCC2)CC1] |
| 148 | 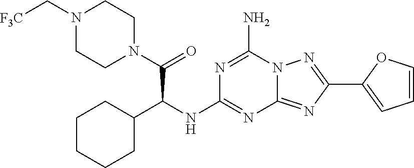 [SMILES: Nc1nc(N[C@H](C(=O)N2CCN(CC(F)(F)F)CC2)C2CCCCC2)nc2nc(-c3ccco3)nn12] |
| 149 | 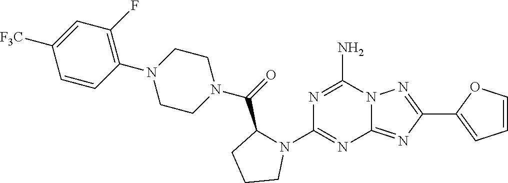 [SMILES: Nc1nc(N2CCC[C@H]2C(=O)N2CCN(c3ccc(C(F)(F)F)cc3F)CC2)nc2nc(-c3ccco3)nn12] |
| 150 | 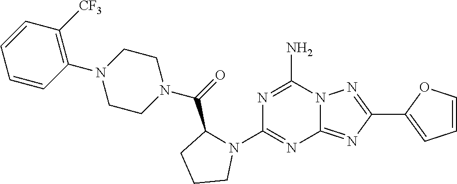 [SMILES: Nc1nc(N2CCC[C@H]2C(=O)N2CCN(c3ccccc3C(F)(F)F)CC2)nc2nc(-c3ccco3)nn12] |
| 151 | 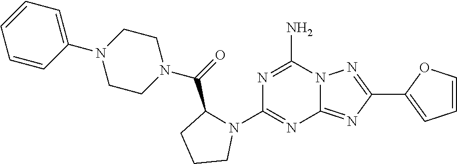 [SMILES: Nc1nc(N2CCC[C@H]2C(=O)N2CCN(c3ccccc3)CC2)nc2nc(-c3ccco3)nn12] |
| 152 | 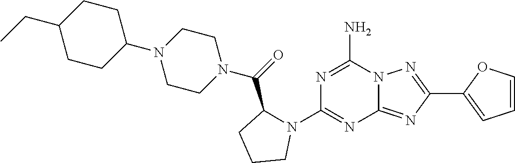 [SMILES: CCC1CCC(N2CCN(C(=O)[C@@H]3CCCN3c3nc(N)n4nc(-c5ccco5)nc4n3)CC2)CC1] |
| 153 | 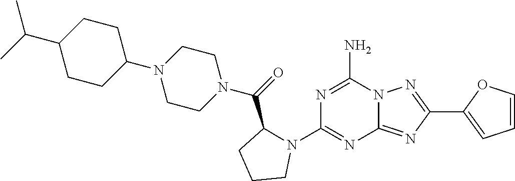 [SMILES: CC(C)C1CCC(N2CCN(C(=O)[C@@H]3CCCN3c3nc(N)n4nc(-c5ccco5)nc4n3)CC2)CC1] |
| 154 | 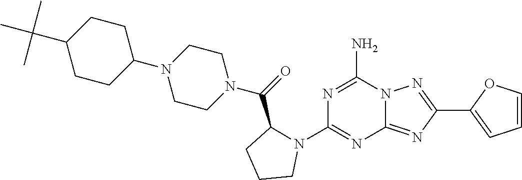 [SMILES: CC(C)(C)C1CCC(N2CCN(C(=O)[C@@H]3CCCN3c3nc(N)n4nc(-c5ccco5)nc4n3)CC2)CC1] |
| 155 | 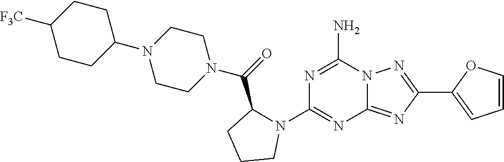 [SMILES: Nc1nc(N2CCC[C@H]2C(=O)N2CCN(C3CCC(C(F)(F)F)CC3)CC2)nc2nc(-c3ccco3)nn12] |
| 156 | 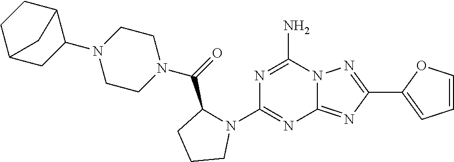 [SMILES: Nc1nc(N2CCC[C@H]2C(=O)N2CCN(C3CC4CCC3C4)CC2)nc2nc(-c3ccco3)nn12] |
| 157 | 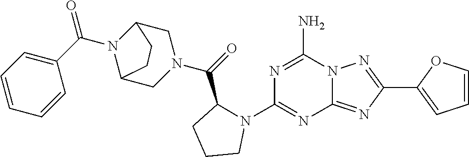 [SMILES: Nc1nc(N2CCC[C@H]2C(=O)N2CC3CCC(C2)N3C(=O)c2ccccc2)nc2nc(-c3ccco3)nn12] |
| 158 | 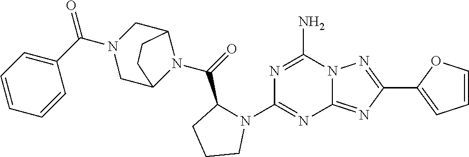 [SMILES: Nc1nc(N2CCC[C@H]2C(=O)N2C3CCC2CN(C(=O)c2ccccc2)C3)nc2nc(-c3ccco3)nn12] |
| 159 | 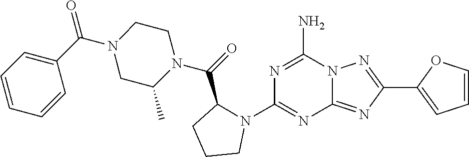 [SMILES: C[C@@H]1CN(C(=O)c2ccccc2)CCN1C(=O)[C@@H]1CCCN1c1nc(N)n2nc(-c3ccco3)nc2n1] |
| 160 | 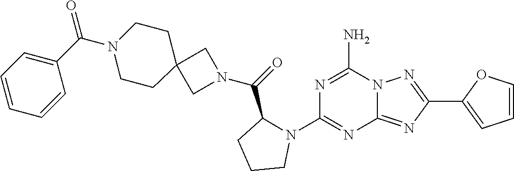 [SMILES: Nc1nc(N2CCC[C@H]2C(=O)N2CC3(CCN(C(=O)c4ccccc4)CC3)C2)nc2nc(-c3ccco3)nn12] |
| 161 | 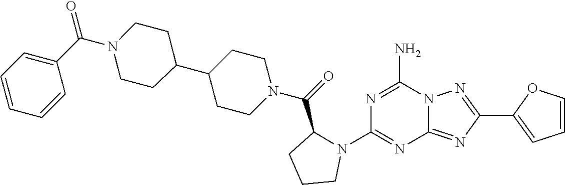 [SMILES: Nc1nc(N2CCC[C@H]2C(=O)N2CCC(C3CCN(C(=O)c4ccccc4)CC3)CC2)nc2nc(-c3ccco3)nn12] |
| 162 | 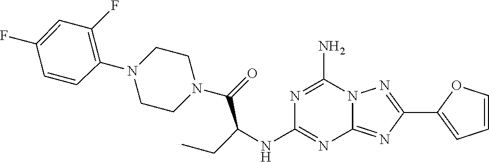 [SMILES: CC[C@H](Nc1nc(N)n2nc(-c3ccco3)nc2n1)C(=O)N1CCN(c2ccc(F)cc2F)CC1] |
| 163 | 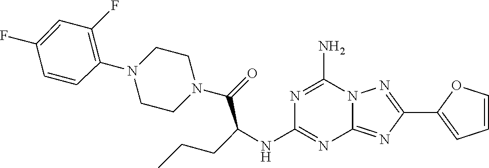 [SMILES: CCC[C@H](Nc1nc(N)n2nc(-c3ccco3)nc2n1)C(=O)N1CCN(c2ccc(F)cc2F)CC1] |
| 164 | 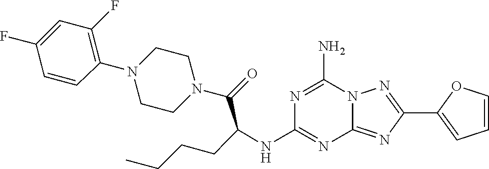 [SMILES: CCCC[C@H](Nc1nc(N)n2nc(-c3ccco3)nc2n1)C(=O)N1CCN(c2ccc(F)cc2F)CC1] |
| 165 | 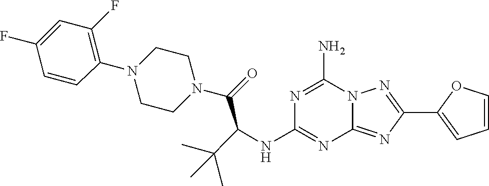 [SMILES: CC(C)(C)[C@H](Nc1nc(N)n2nc(-c3ccco3)nc2n1)C(=O)N1CCN(c2ccc(F)cc2F)CC1] |
| 166 | 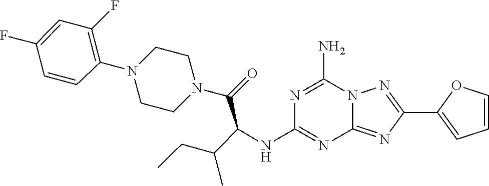 [SMILES: CCC(C)[C@H](Nc1nc(N)n2nc(-c3ccco3)nc2n1)C(=O)N1CCN(c2ccc(F)cc2F)CC1] |
| 167 | 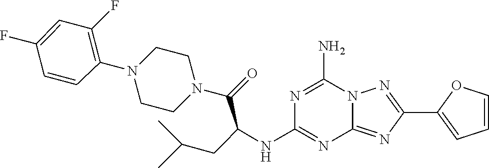 [SMILES: CC(C)C[C@H](Nc1nc(N)n2nc(-c3ccco3)nc2n1)C(=O)N1CCN(c2ccc(F)cc2F)CC1] |
| 168 | 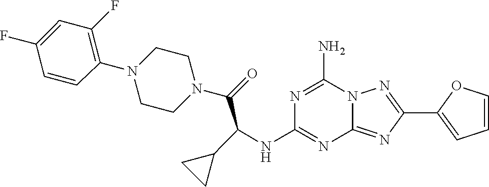 [SMILES: Nc1nc(N[C@H](C(=O)N2CCN(c3ccc(F)cc3F)CC2)C2CC2)nc2nc(-c3ccco3)nn12] |
| 169 | 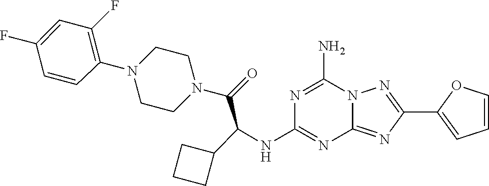 [SMILES: Nc1nc(N[C@H](C(=O)N2CCN(c3ccc(F)cc3F)CC2)C2CCC2)nc2nc(-c3ccco3)nn12] |
| 170 | 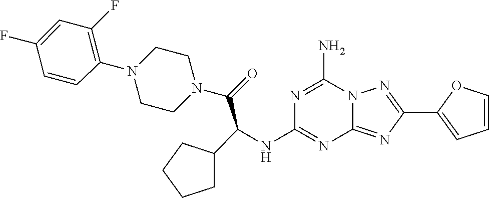 [SMILES: Nc1nc(N[C@H](C(=O)N2CCN(c3ccc(F)cc3F)CC2)C2CCCC2)nc2nc(-c3ccco3)nn12] |
| 171 | 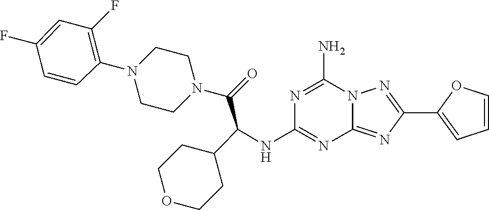 [SMILES: Nc1nc(N[C@H](C(=O)N2CCN(c3ccc(F)cc3F)CC2)C2CCOCC2)nc2nc(-c3ccco3)nn12] |
| 172 | 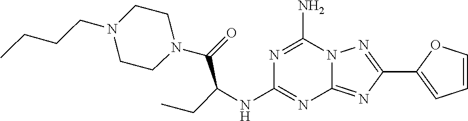 [SMILES: CCCCN1CCN(C(=O)[C@H](CC)Nc2nc(N)n3nc(-c4ccco4)nc3n2)CC1] |
| 173 | 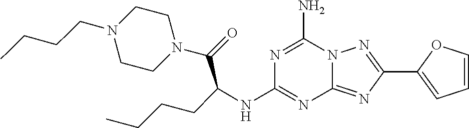 [SMILES: CCCC[C@H](Nc1nc(N)n2nc(-c3ccco3)nc2n1)C(=O)N1CCN(CCCC)CC1] |
| 174 | 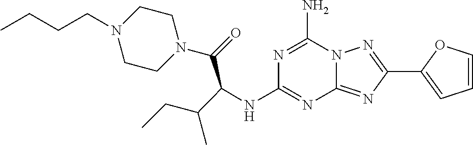 [SMILES: CCCCN1CCN(C(=O)[C@@H](Nc2nc(N)n3nc(-c4ccco4)nc3n2)C(C)CC)CC1] |
| 175 | 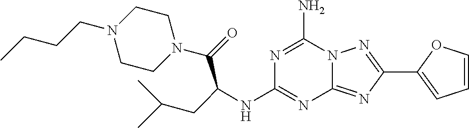 [SMILES: CCCCN1CCN(C(=O)[C@H](CC(C)C)Nc2nc(N)n3nc(-c4ccco4)nc3n2)CC1] |
| 176 | 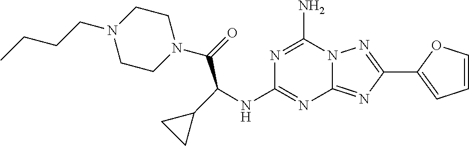 [SMILES: CCCCN1CCN(C(=O)[C@@H](Nc2nc(N)n3nc(-c4ccco4)nc3n2)C2CC2)CC1] |
| 177 | 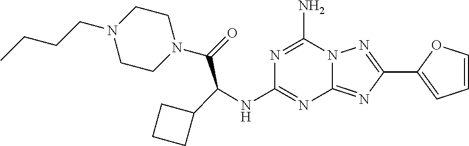 [SMILES: CCCCN1CCN(C(=O)[C@@H](Nc2nc(N)n3nc(-c4ccco4)nc3n2)C2CCC2)CC1] |
| 178 | 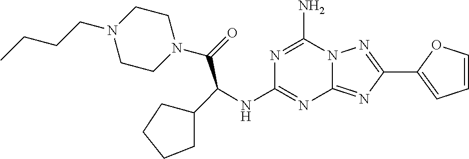 [SMILES: CCCCN1CCN(C(=O)[C@@H](Nc2nc(N)n3nc(-c4ccco4)nc3n2)C2CCCC2)CC1] |
| 179 | 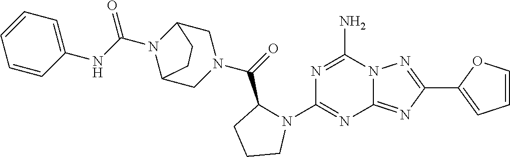 [SMILES: Nc1nc(N2CCC[C@H]2C(=O)N2CC3CCC(C2)N3C(=O)Nc2ccccc2)nc2nc(-c3ccco3)nn12] |
| 180 | 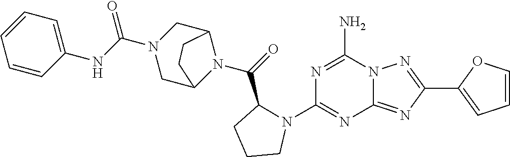 [SMILES: Nc1nc(N2CCC[C@H]2C(=O)N2C3CCC2CN(C(=O)Nc2ccccc2)C3)nc2nc(-c3ccco3)nn12] |
| 181 | 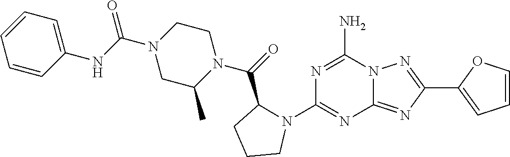 [SMILES: C[C@H]1CN(C(=O)Nc2ccccc2)CCN1C(=O)[C@@H]1CCCN1c1nc(N)n2nc(-c3ccco3)nc2n1] |
| 182 | 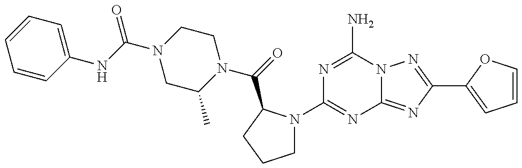 [SMILES: C[C@@H]1CN(C(=O)Nc2ccccc2)CCN1C(=O)[C@@H]1CCCN1c1nc(N)n2nc(-c3ccco3)nc2n1] |
| 183 | 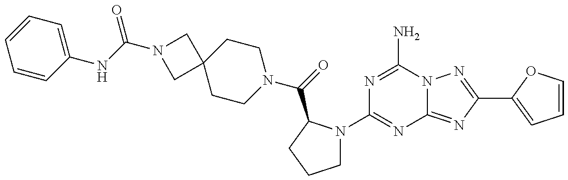 [SMILES: Nc1nc(N2CCC[C@H]2C(=O)N2CCC3(CC2)CN(C(=O)Nc2ccccc2)C3)nc2nc(-c3ccco3)nn12] |
| 184 | 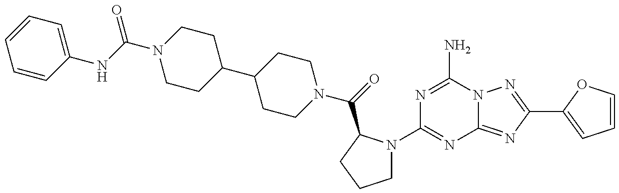 [SMILES: Nc1nc(N2CCC[C@H]2C(=O)N2CCC(C3CCN(C(=O)Nc4ccccc4)CC3)CC2)nc2nc(-c3ccco3)nn12] |
| 185 | 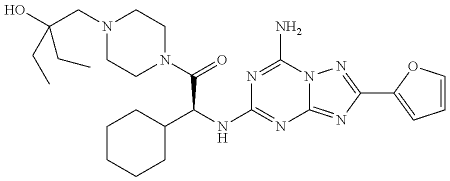 [SMILES: CCC(O)(CC)CN1CCN(C(=O)[C@@H](Nc2nc(N)n3nc(-c4ccco4)nc3n2)C2CCCCC2)CC1] |
| 186 | 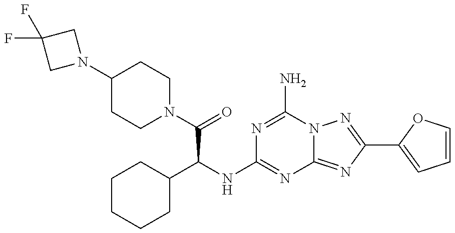 [SMILES: Nc1nc(N[C@H](C(=O)N2CCC(N3CC(F)(F)C3)CC2)C2CCCCC2)nc2nc(-c3ccco3)nn12] |
| 187 | 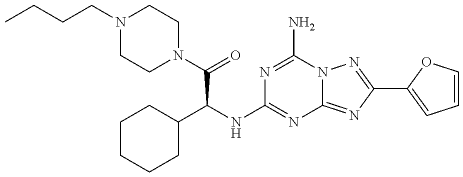 [SMILES: CCCCN1CCN(C(=O)[C@@H](Nc2nc(N)n3nc(-c4ccco4)nc3n2)C2CCCCC2)CC1] |
| 188 | 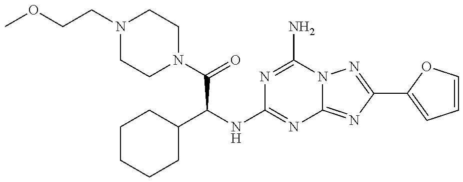 [SMILES: COCCN1CCN(C(=O)[C@@H](Nc2nc(N)n3nc(-c4ccco4)nc3n2)C2CCCCC2)CC1] |
| 189 | 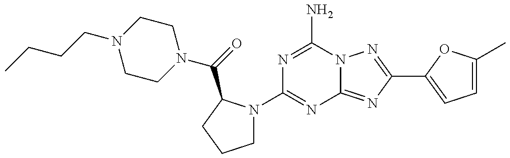 [SMILES: CCCCN1CCN(C(=O)[C@@H]2CCCN2c2nc(N)n3nc(-c4ccc(C)o4)nc3n2)CC1] |
| 190 | 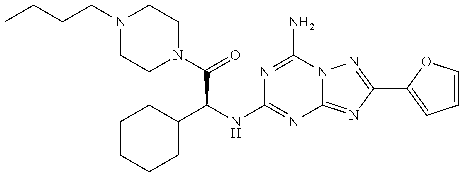 [SMILES: CCCCN1CCN(C(=O)[C@@H](Nc2nc(N)n3nc(-c4ccco4)nc3n2)C2CCCCC2)CC1] |
| 191 | 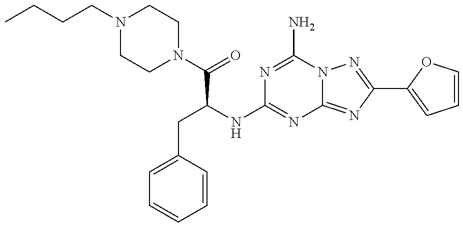 [SMILES: CCCCN1CCN(C(=O)[C@H](Cc2ccccc2)Nc2nc(N)n3nc(-c4ccco4)nc3n2)CC1] |
| 192 | 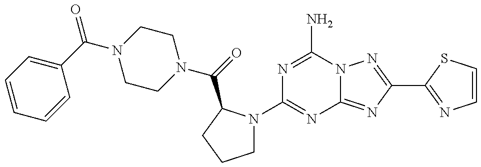 [SMILES: Nc1nc(N2CCC[C@H]2C(=O)N2CCN(C(=O)c3ccccc3)CC2)nc2nc(-c3nccs3)nn12] |
| 193 | 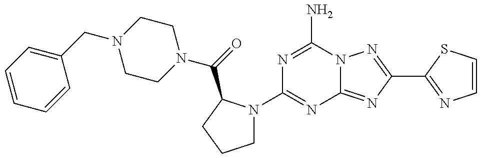 [SMILES: Nc1nc(N2CCC[C@H]2C(=O)N2CCN(Cc3ccccc3)CC2)nc2nc(-c3nccs3)nn12] |
| 194 | 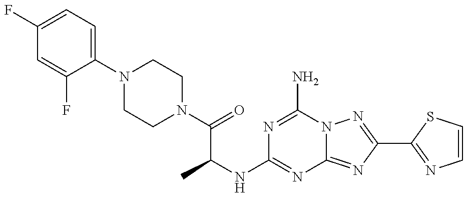 [SMILES: C[C@H](Nc1nc(N)n2nc(-c3nccs3)nc2n1)C(=O)N1CCN(c2ccc(F)cc2F)CC1] |
| 195 | 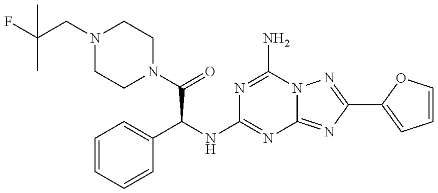 [SMILES: CC(C)(F)CN1CCN(C(=O)[C@@H](Nc2nc(N)n3nc(-c4ccco4)nc3n2)c2ccccc2)CC1] |
| 196 | 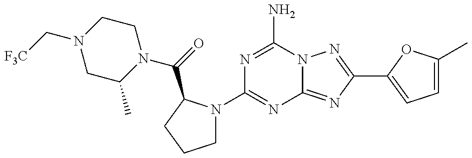 [SMILES: Cc1ccc(-c2nc3nc(N4CCC[C@H]4C(=O)N4CCN(CC(F)(F)F)C[C@H]4C)nc(N)n3n2)o1] |
| 197 | 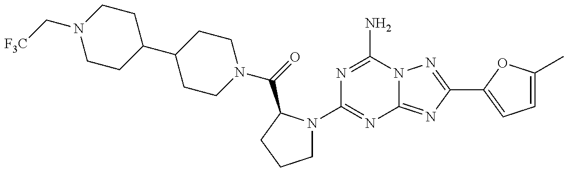 [SMILES: Cc1ccc(-c2nc3nc(N4CCC[C@H]4C(=O)N4CCC(C5CCN(CC(F)(F)F)CC5)CC4)nc(N)n3n2)o1] |
| 198 | 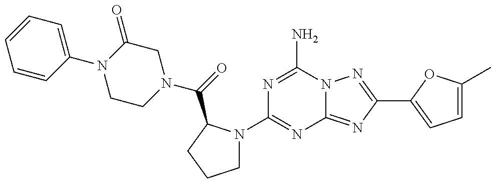 [SMILES: Cc1ccc(-c2nc3nc(N4CCC[C@H]4C(=O)N4CCN(c5ccccc5)C(=O)C4)nc(N)n3n2)o1] |
| 199 | 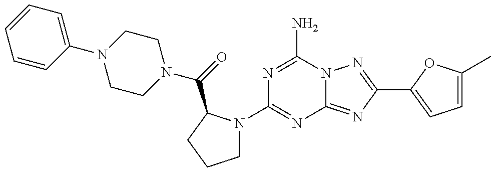 [SMILES: Cc1ccc(-c2nc3nc(N4CCC[C@H]4C(=O)N4CCN(c5ccccc5)CC4)nc(N)n3n2)o1] |
| 200 | 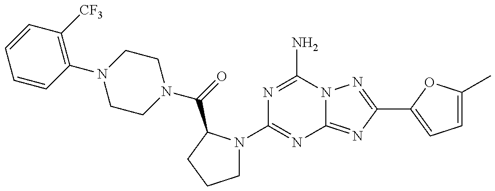 [SMILES: Cc1ccc(-c2nc3nc(N4CCC[C@H]4C(=O)N4CCN(c5ccccc5C(F)(F)F)CC4)nc(N)n3n2)o1] |
| 201 | 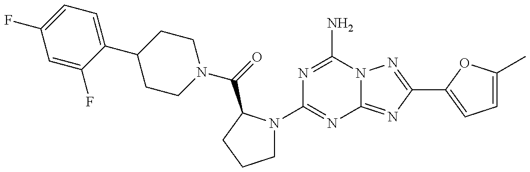 [SMILES: Cc1ccc(-c2nc3nc(N4CCC[C@H]4C(=O)N4CCC(c5ccc(F)cc5F)CC4)nc(N)n3n2)o1] |
| 202 | 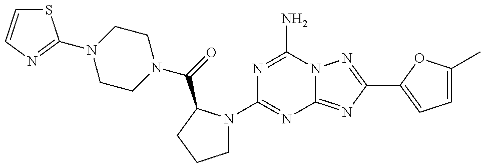 [SMILES: Cc1ccc(-c2nc3nc(N4CCC[C@H]4C(=O)N4CCN(c5nccs5)CC4)nc(N)n3n2)o1] |
| 203 | 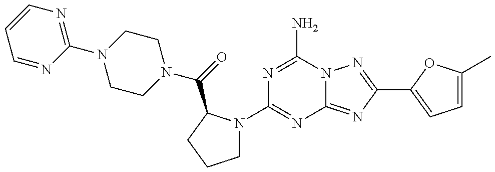 [SMILES: Cc1ccc(-c2nc3nc(N4CCC[C@H]4C(=O)N4CCN(c5ncccn5)CC4)nc(N)n3n2)o1] |
| 204 | 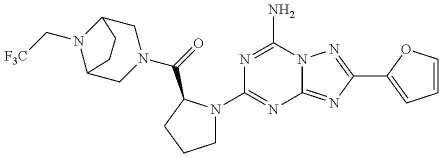 [SMILES: Nc1nc(N2CCC[C@H]2C(=O)N2CC3CCC(C2)N3CC(F)(F)F)nc2nc(-c3ccco3)nn12] |
| 205 | 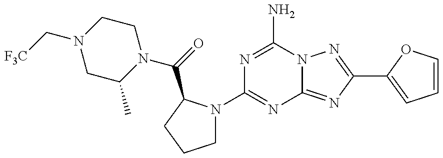 [SMILES: C[C@@H]1CN(CC(F)(F)F)CCN1C(=O)[C@@H]1CCCN1c1nc(N)n2nc(-c3ccco3)nc2n1] |
| 206 | 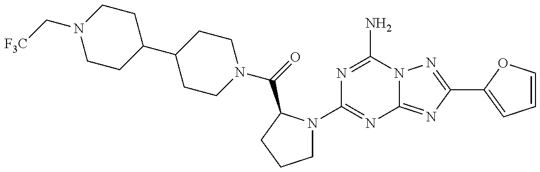 [SMILES: Nc1nc(N2CCC[C@H]2C(=O)N2CCC(C3CCN(CC(F)(F)F)CC3)CC2)nc2nc(-c3ccco3)nn12] |
| 207 | 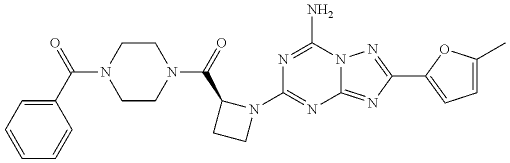 [SMILES: Cc1ccc(-c2nc3nc(N4CC[C@H]4C(=O)N4CCN(C(=O)c5ccccc5)CC4)nc(N)n3n2)o1] |
| 208 | 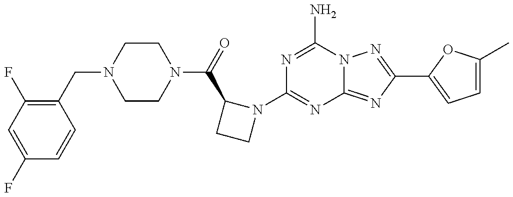 [SMILES: Cc1ccc(-c2nc3nc(N4CC[C@H]4C(=O)N4CCN(Cc5ccc(F)cc5F)CC4)nc(N)n3n2)o1] |
| 209 | 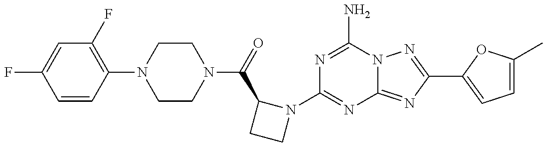 [SMILES: Cc1ccc(-c2nc3nc(N4CC[C@H]4C(=O)N4CCN(c5ccc(F)cc5F)CC4)nc(N)n3n2)o1] |
| 210 | 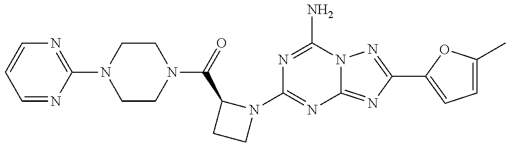 [SMILES: Cc1ccc(-c2nc3nc(N4CC[C@H]4C(=O)N4CCN(c5ncccn5)CC4)nc(N)n3n2)o1] |
| 211 | 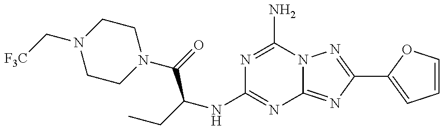 [SMILES: CC[C@H](Nc1nc(N)n2nc(-c3ccco3)nc2n1)C(=O)N1CCN(CC(F)(F)F)CC1] |
| 212 | 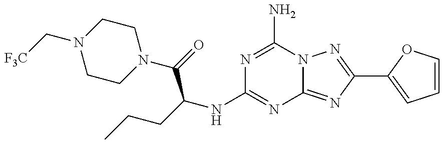 [SMILES: CCC[C@H](Nc1nc(N)n2nc(-c3ccco3)nc2n1)C(=O)N1CCN(CC(F)(F)F)CC1] |
| 213 | 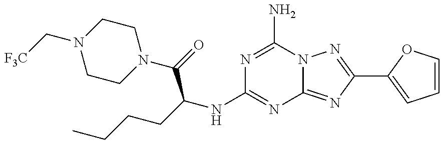 [SMILES: CCCC[C@H](Nc1nc(N)n2nc(-c3ccco3)nc2n1)C(=O)N1CCN(CC(F)(F)F)CC1] |
| 214 | 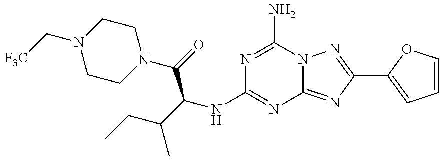 [SMILES: CCC(C)[C@H](Nc1nc(N)n2nc(-c3ccco3)nc2n1)C(=O)N1CCN(CC(F)(F)F)CC1] |
| 215 | 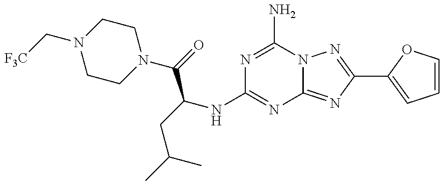 [SMILES: CC(C)C[C@H](Nc1nc(N)n2nc(-c3ccco3)nc2n1)C(=O)N1CCN(CC(F)(F)F)CC1] |
| 216 | 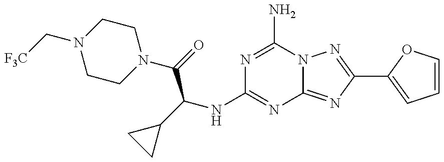 [SMILES: Nc1nc(N[C@H](C(=O)N2CCN(CC(F)(F)F)CC2)C2CC2)nc2nc(-c3ccco3)nn12] |
| 217 | 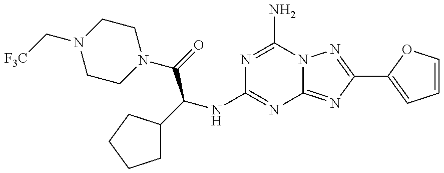 [SMILES: Nc1nc(N[C@H](C(=O)N2CCN(CC(F)(F)F)CC2)C2CCCC2)nc2nc(-c3ccco3)nn12] |
| 218 | 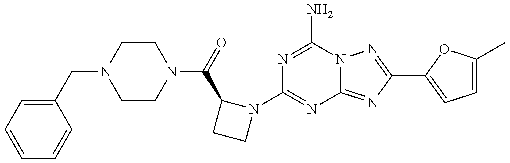 [SMILES: Cc1ccc(-c2nc3nc(N4CC[C@H]4C(=O)N4CCN(Cc5ccccc5)CC4)nc(N)n3n2)o1] |
| 219 | 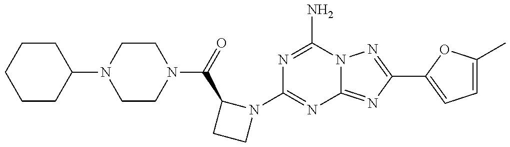 [SMILES: Cc1ccc(-c2nc3nc(N4CC[C@H]4C(=O)N4CCN(C5CCCCC5)CC4)nc(N)n3n2)o1] |
| 220 | 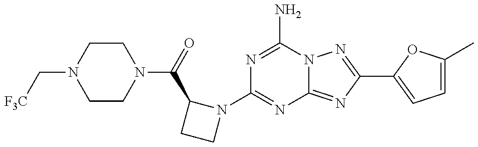 [SMILES: Cc1ccc(-c2nc3nc(N4CC[C@H]4C(=O)N4CCN(CC(F)(F)F)CC4)nc(N)n3n2)o1] |
| 221 | 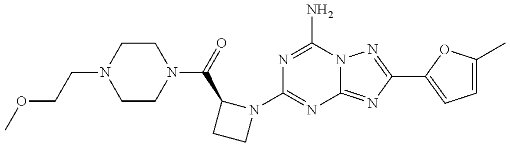 |
| 222 | 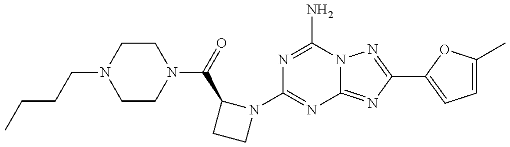 [SMILES: CCCCN1CCN(C(=O)[C@@H]2CCN2c2nc(N)n3nc(-c4ccc(C)o4)nc3n2)CC1] |
| 223 | 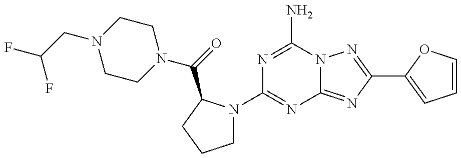 [SMILES: Nc1nc(N2CCC[C@H]2C(=O)N2CCN(CC(F)F)CC2)nc2nc(-c3ccco3)nn12] |
| 224 | 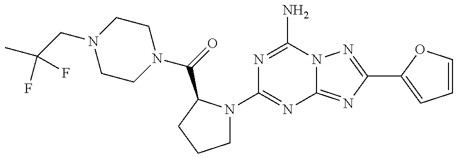 [SMILES: CC(F)(F)CN1CCN(C(=O)[C@@H]2CCCN2c2nc(N)n3nc(-c4ccco4)nc3n2)CC1] |
| 225 | 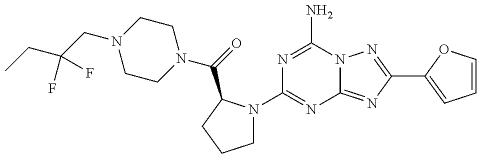 [SMILES: CCC(F)(F)CN1CCN(C(=O)[C@@H]2CCCN2c2nc(N)n3nc(-c4ccco4)nc3n2)CC1] |
| 226 | 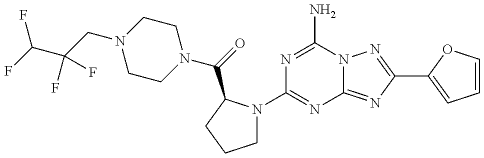 [SMILES: Nc1nc(N2CCC[C@H]2C(=O)N2CCN(CC(F)(F)C(F)F)CC2)nc2nc(-c3ccco3)nn12] |
| 227 | 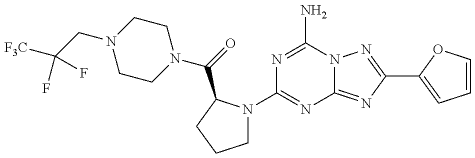 [SMILES: Nc1nc(N2CCC[C@H]2C(=O)N2CCN(CC(F)(F)C(F)(F)F)CC2)nc2nc(-c3ccco3)nn12] |
| 228 | 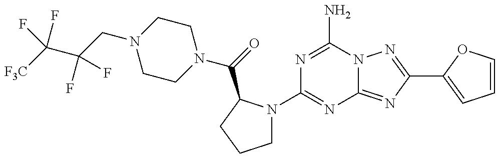 [SMILES: Nc1nc(N2CCC[C@H]2C(=O)N2CCN(CC(F)(F)C(F)(F)C(F)(F)F)CC2)nc2nc(-c3ccco3)nn12] |
| 229 | 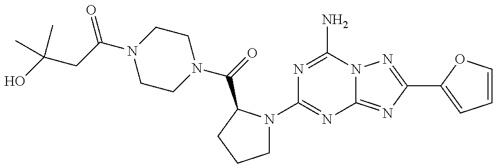 [SMILES: CC(C)(O)CC(=O)N1CCN(C(=O)[C@@H]2CCCN2c2nc(N)n3nc(-c4ccco4)nc3n2)CC1] |
| 230 | 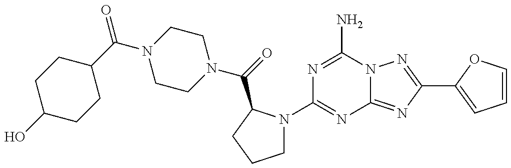 [SMILES: Nc1nc(N2CCC[C@H]2C(=O)N2CCN(C(=O)C3CCC(O)CC3)CC2)nc2nc(-c3ccco3)nn12] |
| 231 | 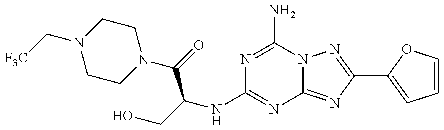 [SMILES: Nc1nc(N[C@@H](CO)C(=O)N2CCN(CC(F)(F)F)CC2)nc2nc(-c3ccco3)nn12] |
| 232 | 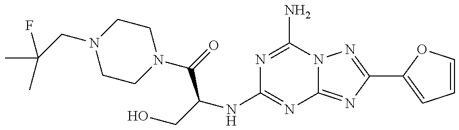 [SMILES: CC(C)(F)CN1CCN(C(=O)[C@H](CO)Nc2nc(N)n3nc(-c4ccco4)nc3n2)CC1] |
| 233 | 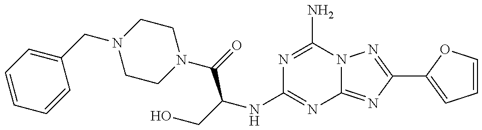 [SMILES: Nc1nc(N[C@@H](CO)C(=O)N2CCN(Cc3ccccc3)CC2)nc2nc(-c3ccco3)nn12] |
| 234 | 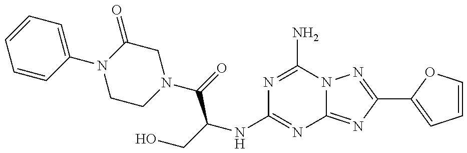 [SMILES: Nc1nc(N[C@@H](CO)C(=O)N2CCN(c3ccccc3)C(=O)C2)nc2nc(-c3ccco3)nn12] |
| 235 | 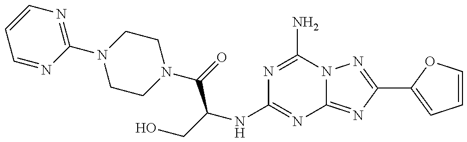 [SMILES: Nc1nc(N[C@@H](CO)C(=O)N2CCN(c3ncccn3)CC2)nc2nc(-c3ccco3)nn12] |
| 236 | 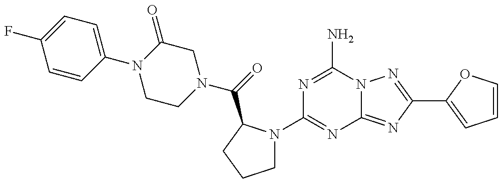 [SMILES: Nc1nc(N2CCC[C@H]2C(=O)N2CCN(c3ccc(F)cc3)C(=O)C2)nc2nc(-c3ccco3)nn12] |
| 237 | 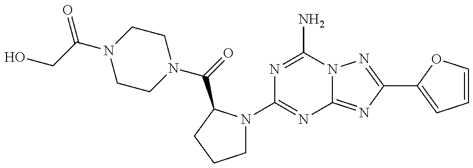 [SMILES: Nc1nc(N2CCC[C@H]2C(=O)N2CCN(C(=O)CO)CC2)nc2nc(-c3ccco3)nn12] |
| 238 | 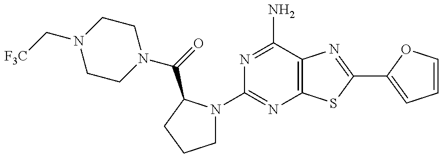 [SMILES: Nc1nc(N2CCC[C@H]2C(=O)N2CCN(CC(F)(F)F)CC2)nc2sc(-c3ccco3)nc12] |
| 239 | 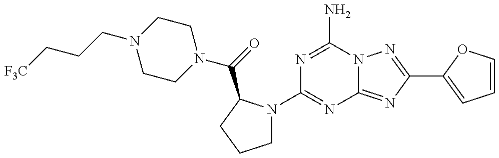 [SMILES: Nc1nc(N2CCC[C@H]2C(=O)N2CCN(CCCC(F)(F)F)CC2)nc2nc(-c3ccco3)nn12] |
| 240 | 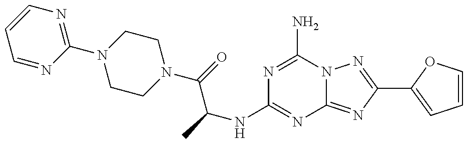 [SMILES: C[C@H](Nc1nc(N)n2nc(-c3ccco3)nc2n1)C(=O)N1CCN(c2ncccn2)CC1] |
| 241 | 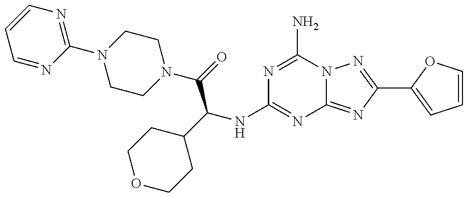 [SMILES: Nc1nc(N[C@H](C(=O)N2CCN(c3ncccn3)CC2)C2CCOCC2)nc2nc(-c3ccco3)nn12] |
| 242 | 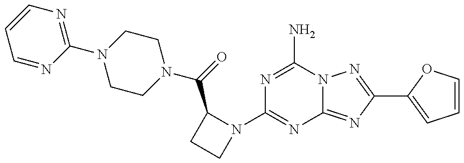 [SMILES: Nc1nc(N2CC[C@H]2C(=O)N2CCN(c3ncccn3)CC2)nc2nc(-c3ccco3)nn12] |
| 243 | 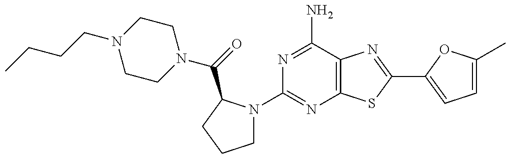 [SMILES: CCCCN1CCN(C(=O)[C@@H]2CCCN2c2nc(N)c3nc(-c4ccc(C)o4)sc3n2)CC1] |
| 244 | 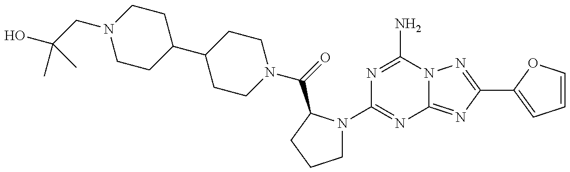 [SMILES: CC(C)(O)CN1CCC(C2CCN(C(=O)[C@@H]3CCCN3c3nc(N)n4nc(-c5ccco5)nc4n3)CC2)CC1] |
| 245 | 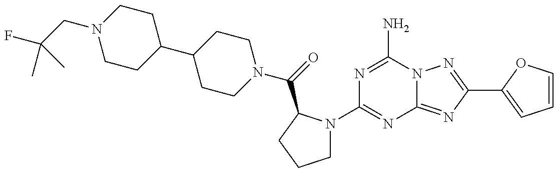 [SMILES: CC(C)(F)CN1CCC(C2CCN(C(=O)[C@@H]3CCCN3c3nc(N)n4nc(-c5ccco5)nc4n3)CC2)CC1] |
| 246 | 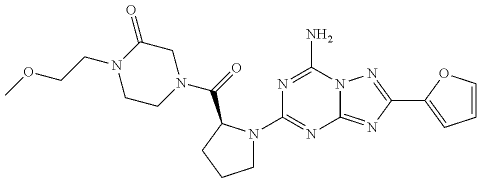 [SMILES: COCCN1CCN(C(=O)[C@@H]2CCCN2c2nc(N)n3nc(-c4ccco4)nc3n2)CC1=O] |
| 247 | 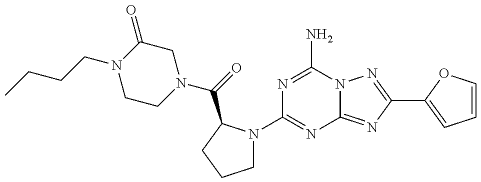 [SMILES: CCCCN1CCN(C(=O)[C@@H]2CCCN2c2nc(N)n3nc(-c4ccco4)nc3n2)CC1=O] |
| 248 | 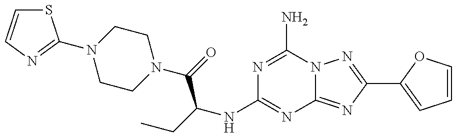 [SMILES: CC[C@H](Nc1nc(N)n2nc(-c3ccco3)nc2n1)C(=O)N1CCN(c2nccs2)CC1] |
| 249 | 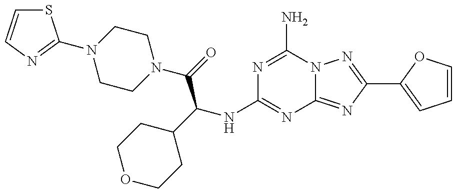 [SMILES: Nc1nc(N[C@H](C(=O)N2CCN(c3nccs3)CC2)C2CCOCC2)nc2nc(-c3ccco3)nn12] |
| 250 | 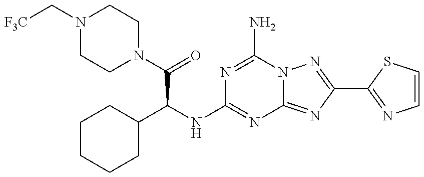 [SMILES: Nc1nc(N[C@H](C(=O)N2CCN(CC(F)(F)F)CC2)C2CCCCC2)nc2nc(-c3nccs3)nn12] |
| 251 | 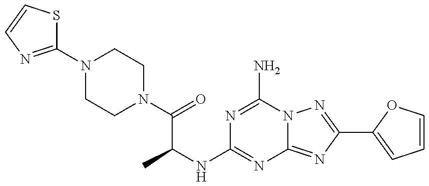 [SMILES: C[C@H](Nc1nc(N)n2nc(-c3ccco3)nc2n1)C(=O)N1CCN(c2nccs2)CC1] |
| 252 | 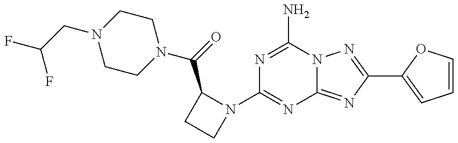 [SMILES: Nc1nc(N2CC[C@H]2C(=O)N2CCN(CC(F)F)CC2)nc2nc(-c3ccco3)nn12] |
| 253 | 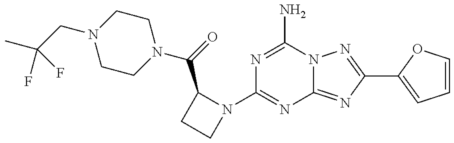 [SMILES: CC(F)(F)CN1CCN(C(=O)[C@@H]2CCN2c2nc(N)n3nc(-c4ccco4)nc3n2)CC1] |
| 254 | 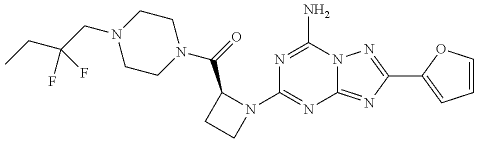 [SMILES: CCC(F)(F)CN1CCN(C(=O)[C@@H]2CCN2c2nc(N)n3nc(-c4ccco4)nc3n2)CC1] |
| 255 | 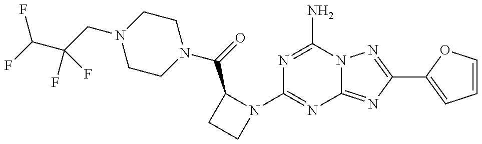 [SMILES: Nc1nc(N2CC[C@H]2C(=O)N2CCN(CC(F)(F)C(F)F)CC2)nc2nc(-c3ccco3)nn12] |
| 256 | 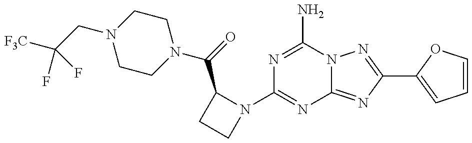 [SMILES: Nc1nc(N2CC[C@H]2C(=O)N2CCN(CC(F)(F)C(F)(F)F)CC2)nc2nc(-c3ccco3)nn12] |
| 257 | 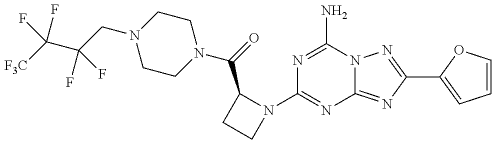 [SMILES: Nc1nc(N2CC[C@H]2C(=O)N2CCN(CC(F)(F)C(F)(F)C(F)(F)F)CC2)nc2nc(-c3ccco3)nn12] |
| 258 | 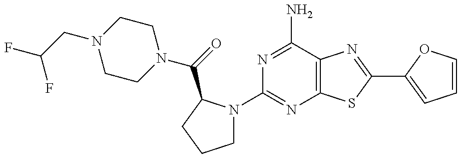 [SMILES: Nc1nc(N2CCC[C@H]2C(=O)N2CCN(CC(F)F)CC2)nc2sc(-c3ccco3)nc12] |
| 259 | 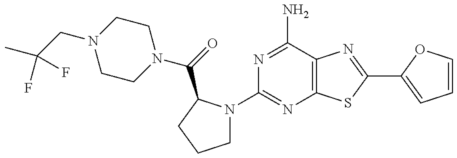 [SMILES: CC(F)(F)CN1CCN(C(=O)[C@@H]2CCCN2c2nc(N)c3nc(-c4ccco4)sc3n2)CC1] |
| 260 | 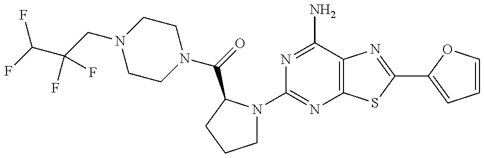 [SMILES: Nc1nc(N2CCC[C@H]2C(=O)N2CCN(CC(F)(F)C(F)F)CC2)nc2sc(-c3ccco3)nc12] |
| 261 | 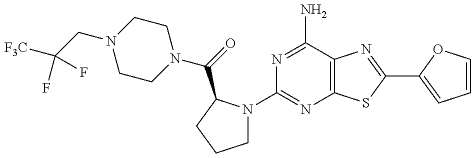 [SMILES: Nc1nc(N2CCC[C@H]2C(=O)N2CCN(CC(F)(F)C(F)(F)F)CC2)nc2sc(-c3ccco3)nc12] |
| 262 | 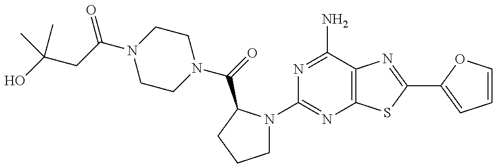 [SMILES: CC(C)(O)CC(=O)N1CCN(C(=O)[C@@H]2CCCN2c2nc(N)c3nc(-c4ccco4)sc3n2)CC1] |
| 263 | 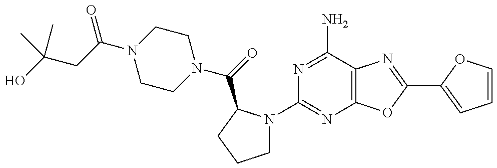 [SMILES: CC(C)(O)CC(=O)N1CCN(C(=O)[C@@H]2CCCN2c2nc(N)c3nc(-c4ccco4)oc3n2)CC1] |
| 264 | 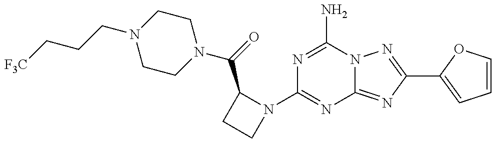 [SMILES: Nc1nc(N2CC[C@H]2C(=O)N2CCN(CCCC(F)(F)F)CC2)nc2nc(-c3ccco3)nn12] |
| 265 | 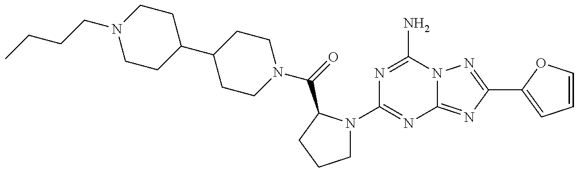 [SMILES: CCCCN1CCC(C2CCN(C(=O)[C@@H]3CCCN3c3nc(N)n4nc(-c5ccco5)nc4n3)CC2)CC1] |
| 266 | 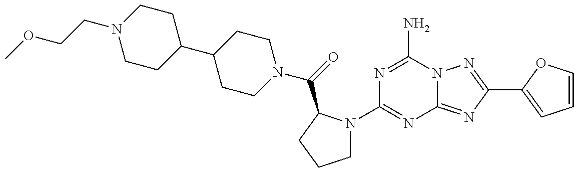 [SMILES: COCCN1CCC(C2CCN(C(=O)[C@@H]3CCCN3c3nc(N)n4nc(-c5ccco5)nc4n3)CC2)CC1] |
| 267 | 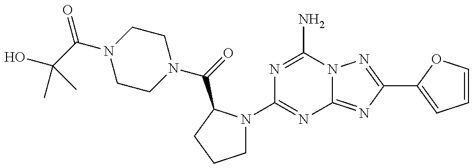 [SMILES: CC(C)(O)C(=O)N1CCN(C(=O)[C@@H]2CCCN2c2nc(N)n3nc(-c4ccco4)nc3n2)CC1] |
| 268 | 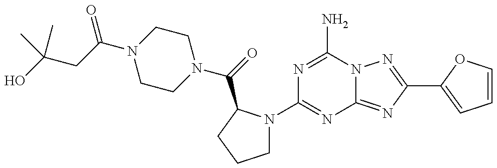 [SMILES: CC(C)(O)CC(=O)N1CCN(C(=O)[C@@H]2CCCN2c2nc(N)n3nc(-c4ccco4)nc3n2)CC1] |
| 269 | 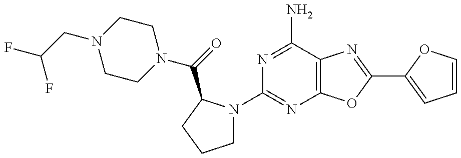 [SMILES: Nc1nc(N2CCC[C@H]2C(=O)N2CCN(CC(F)F)CC2)nc2oc(-c3ccco3)nc12] |
| 270 | 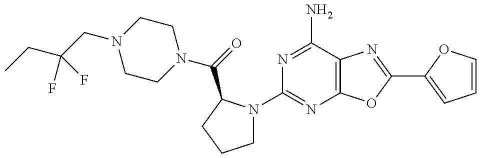 [SMILES: CCC(F)(F)CN1CCN(C(=O)[C@@H]2CCCN2c2nc(N)c3nc(-c4ccco4)oc3n2)CC1] |
| 271 | 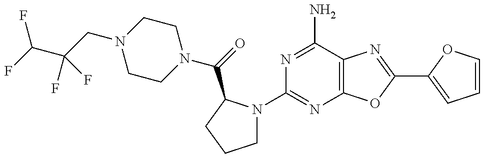 [SMILES: Nc1nc(N2CCC[C@H]2C(=O)N2CCN(CC(F)(F)C(F)F)CC2)nc2oc(-c3ccco3)nc12] |
| 272 | 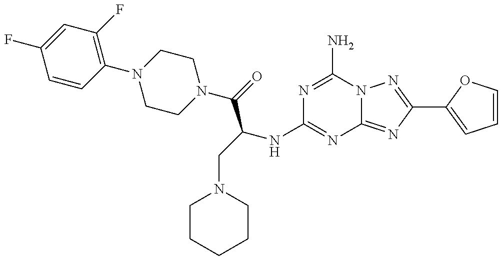 [SMILES: Nc1nc(N[C@@H](CN2CCCCC2)C(=O)N2CCN(c3ccc(F)cc3F)CC2)nc2nc(-c3ccco3)nn12] |
| 273 | 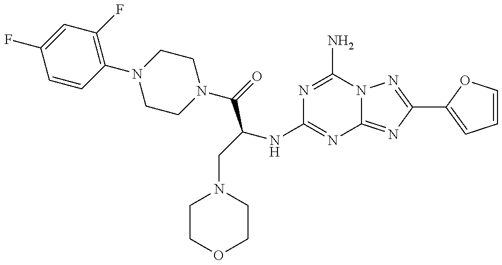 [SMILES: Nc1nc(N[C@@H](CN2CCOCC2)C(=O)N2CCN(c3ccc(F)cc3F)CC2)nc2nc(-c3ccco3)nn12] |
| 274 | 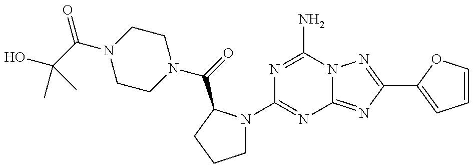 [SMILES: CC(C)(O)C(=O)N1CCN(C(=O)[C@@H]2CCCN2c2nc(N)n3nc(-c4ccco4)nc3n2)CC1] |
| 275 | 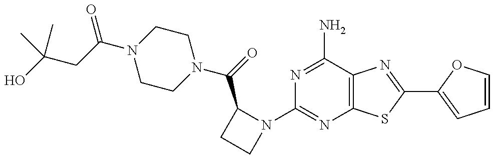 [SMILES: CC(C)(O)CC(=O)N1CCN(C(=O)[C@@H]2CCN2c2nc(N)c3nc(-c4ccco4)sc3n2)CC1] |
| 276 | 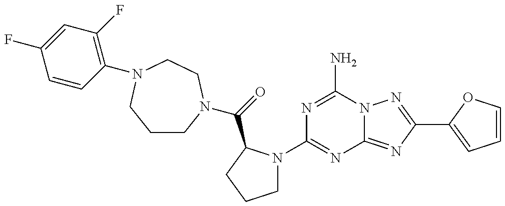 [SMILES: Nc1nc(N2CCC[C@H]2C(=O)N2CCCN(c3ccc(F)cc3F)CC2)nc2nc(-c3ccco3)nn12] |
| 277 | 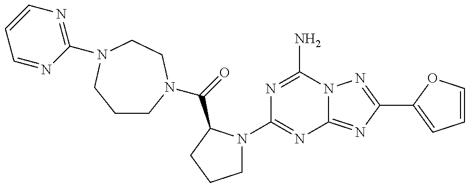 [SMILES: Nc1nc(N2CCC[C@H]2C(=O)N2CCCN(c3ncccn3)CC2)nc2nc(-c3ccco3)nn12] |
| 278 | 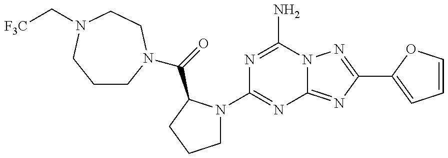 [SMILES: Nc1nc(N2CCC[C@H]2C(=O)N2CCCN(CC(F)(F)F)CC2)nc2nc(-c3ccco3)nn12] |
| 279 | 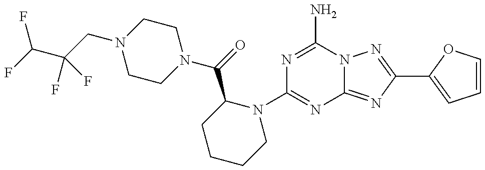 [SMILES: Nc1nc(N2CCCC[C@H]2C(=O)N2CCN(CC(F)(F)C(F)F)CC2)nc2nc(-c3ccco3)nn12] |
| 280 | 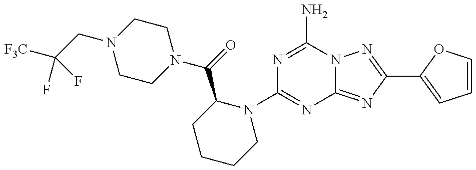 [SMILES: Nc1nc(N2CCCC[C@H]2C(=O)N2CCN(CC(F)(F)C(F)(F)F)CC2)nc2nc(-c3ccco3)nn12] |
| 281 | 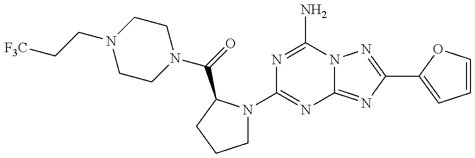 [SMILES: Nc1nc(N2CCC[C@H]2C(=O)N2CCN(CCC(F)(F)F)CC2)nc2nc(-c3ccco3)nn12] |
| 282 | 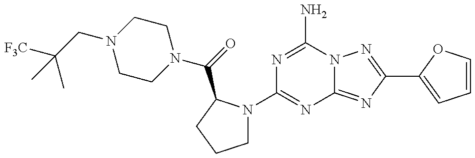 [SMILES: CC(C)(CN1CCN(C(=O)[C@@H]2CCCN2c2nc(N)n3nc(-c4ccco4)nc3n2)CC1)C(F)(F)F] |
| 283 | 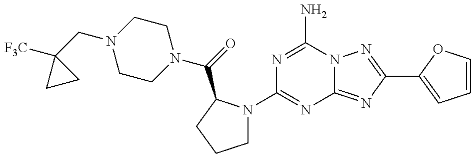 [SMILES: Nc1nc(N2CCC[C@H]2C(=O)N2CCN(CC3(C(F)(F)F)CC3)CC2)nc2nc(-c3ccco3)nn12] |
| 284 | 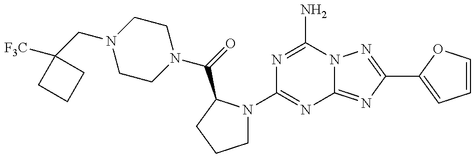 [SMILES: Nc1nc(N2CCC[C@H]2C(=O)N2CCN(CC3(C(F)(F)F)CCC3)CC2)nc2nc(-c3ccco3)nn12] |
| 285 | 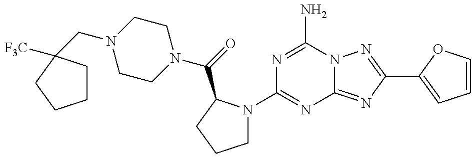 [SMILES: Nc1nc(N2CCC[C@H]2C(=O)N2CCN(CC3(C(F)(F)F)CCCC3)CC2)nc2nc(-c3ccco3)nn12] |
| 286 | 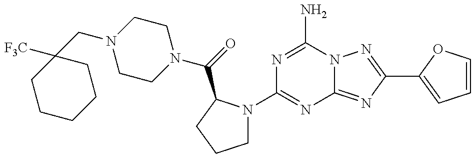 [SMILES: Nc1nc(N2CCC[C@H]2C(=O)N2CCN(CC3(C(F)(F)F)CCCCC3)CC2)nc2nc(-c3ccco3)nn12] |
| 287 | 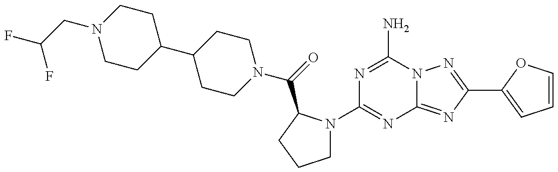 [SMILES: Nc1nc(N2CCC[C@H]2C(=O)N2CCC(C3CCN(CC(F)F)CC3)CC2)nc2nc(-c3ccco3)nn12] |
| 288 | 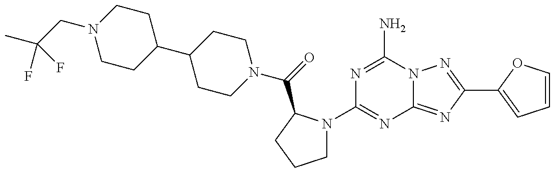 [SMILES: CC(F)(F)CN1CCC(C2CCN(C(=O)[C@@H]3CCCN3c3nc(N)n4nc(-c5ccco5)nc4n3)CC2)CC1] |
| 289 | 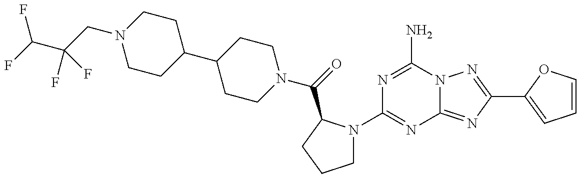 [SMILES: Nc1nc(N2CCC[C@H]2C(=O)N2CCC(C3CCN(CC(F)(F)C(F)F)CC3)CC2)nc2nc(-c3ccco3)nn12] |
| 290 | 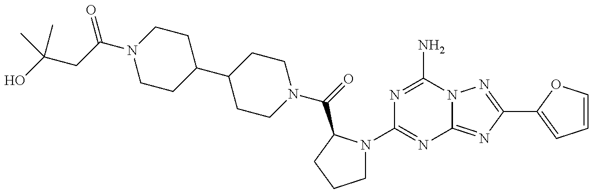 [SMILES: CC(C)(O)CC(=O)N1CCC(C2CCN(C(=O)[C@@H]3CCCN3c3nc(N)n4nc(-c5ccco5)nc4n3)CC2)CC1] |
| 291 | 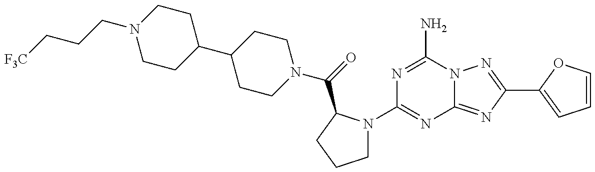 [SMILES: Nc1nc(N2CCC[C@H]2C(=O)N2CCC(C3CCN(CCCC(F)(F)F)CC3)CC2)nc2nc(-c3ccco3)nn12] |
| 292 | 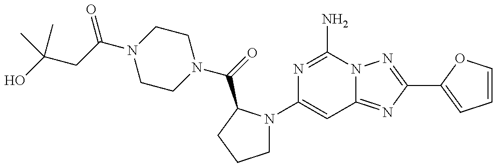 [SMILES: CC(C)(O)CC(=O)N1CCN(C(=O)[C@@H]2CCCN2c2cc3nc(-c4ccco4)nn3c(N)n2)CC1] |
| 293 | 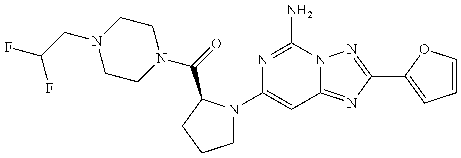 [SMILES: Nc1nc(N2CCC[C@H]2C(=O)N2CCN(CC(F)F)CC2)cc2nc(-c3ccco3)nn12] |
| 294 | 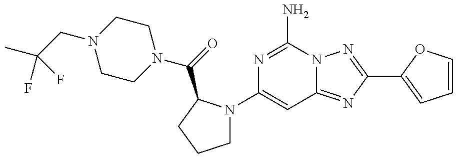 [SMILES: CC(F)(F)CN1CCN(C(=O)[C@@H]2CCCN2c2cc3nc(-c4ccco4)nn3c(N)n2)CC1] |
| 295 | 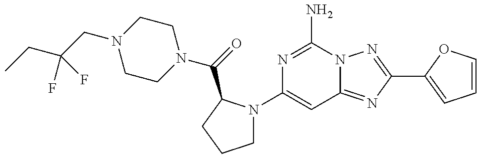 [SMILES: CCC(F)(F)CN1CCN(C(=O)[C@@H]2CCCN2c2cc3nc(-c4ccco4)nn3c(N)n2)CC1] |
| 296 | 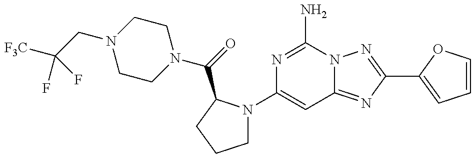 [SMILES: Nc1nc(N2CCC[C@H]2C(=O)N2CCN(CC(F)(F)C(F)(F)F)CC2)cc2nc(-c3ccco3)nn12] |
| 297 | 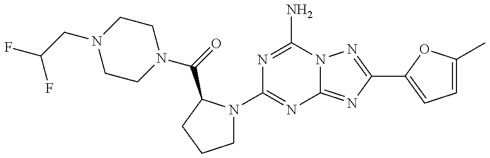 [SMILES: Cc1ccc(-c2nc3nc(N4CCC[C@H]4C(=O)N4CCN(CC(F)F)CC4)nc(N)n3n2)o1] |
| 298 | 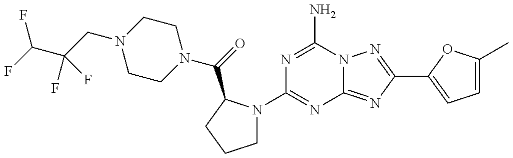 [SMILES: Cc1ccc(-c2nc3nc(N4CCC[C@H]4C(=O)N4CCN(CC(F)(F)C(F)F)CC4)nc(N)n3n2)o1] |
| 299 | 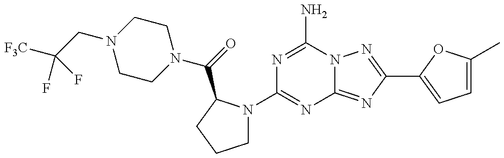 [SMILES: Cc1ccc(-c2nc3nc(N4CCC[C@H]4C(=O)N4CCN(CC(F)(F)C(F)(F)F)CC4)nc(N)n3n2)o1] |
| 300 | 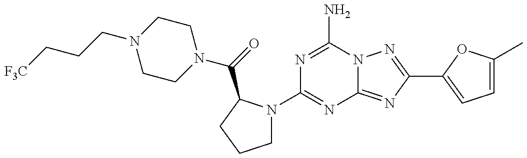 [SMILES: Cc1ccc(-c2nc3nc(N4CCC[C@H]4C(=O)N4CCN(CCCC(F)(F)F)CC4)nc(N)n3n2)o1] |
| 301 | 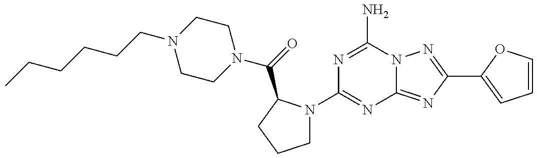 [SMILES: CCCCCCN1CCN(C(=O)[C@@H]2CCCN2c2nc(N)n3nc(-c4ccco4)nc3n2)CC1] |
| 302 | 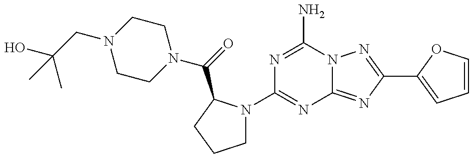 [SMILES: CC(C)(O)CN1CCN(C(=O)[C@@H]2CCCN2c2nc(N)n3nc(-c4ccco4)nc3n2)CC1] |
| 303 | 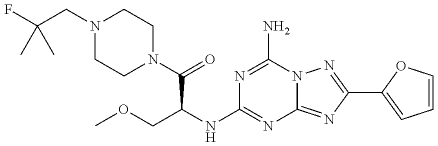 [SMILES: COC[C@H](Nc1nc(N)n2nc(-c3ccco3)nc2n1)C(=O)N1CCN(CC(C)(C)F)CC1] |
| 304 | 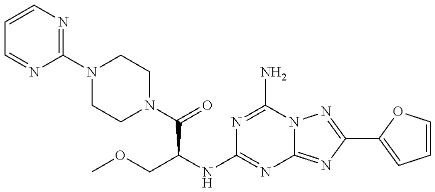 [SMILES: COC[C@H](Nc1nc(N)n2nc(-c3ccco3)nc2n1)C(=O)N1CCN(c2ncccn2)CC1] |
| 305 | 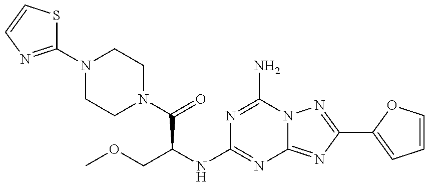 [SMILES: COC[C@H](Nc1nc(N)n2nc(-c3ccco3)nc2n1)C(=O)N1CCN(c2nccs2)CC1] |
| 306 | 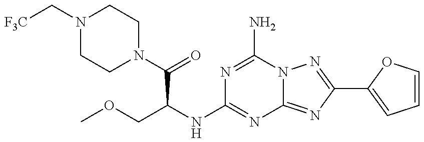 [SMILES: COC[C@H](Nc1nc(N)n2nc(-c3ccco3)nc2n1)C(=O)N1CCN(CC(F)(F)F)CC1] |
| 307 | 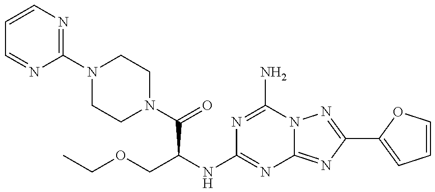 [SMILES: CCOC[C@H](Nc1nc(N)n2nc(-c3ccco3)nc2n1)C(=O)N1CCN(c2ncccn2)CC1] |
| 308 | 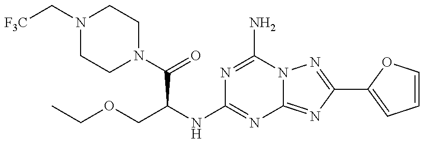 [SMILES: CCOC[C@H](Nc1nc(N)n2nc(-c3ccco3)nc2n1)C(=O)N1CCN(CC(F)(F)F)CC1] |
| 309 | 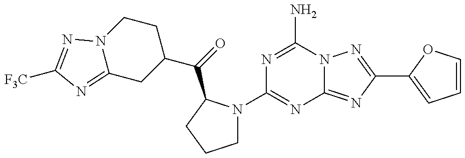 [SMILES: Nc1nc(N2CCC[C@H]2C(=O)C2CCn3nc(C(F)(F)F)nc3C2)nc2nc(-c3ccco3)nn12] |
| 310 | 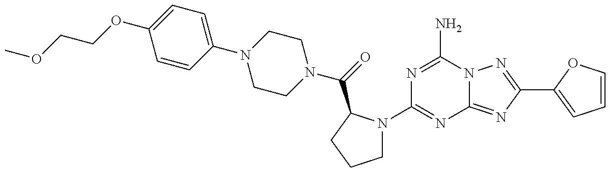 [SMILES: COCCOc1ccc(N2CCN(C(=O)[C@@H]3CCCN3c3nc(N)n4nc(-c5ccco5)nc4n3)CC2)cc1] |
| 311 | 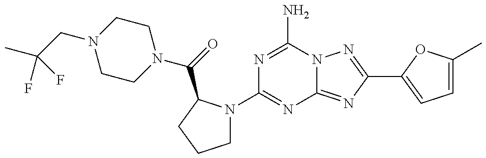 [SMILES: Cc1ccc(-c2nc3nc(N4CCC[C@H]4C(=O)N4CCN(CC(C)(F)F)CC4)nc(N)n3n2)o1] |
| 312 | 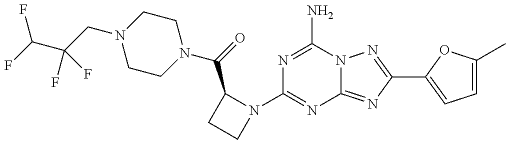 [SMILES: Cc1ccc(-c2nc3nc(N4CC[C@H]4C(=O)N4CCN(CC(F)(F)C(F)F)CC4)nc(N)n3n2)o1] |
| 313 | 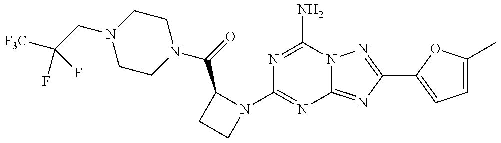 [SMILES: Cc1ccc(-c2nc3nc(N4CC[C@H]4C(=O)N4CCN(CC(F)(F)C(F)(F)F)CC4)nc(N)n3n2)o1] |
| 314 | 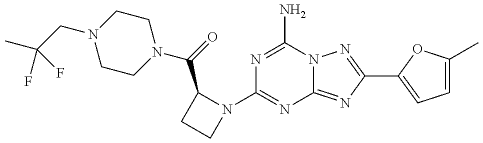 [SMILES: Cc1ccc(-c2nc3nc(N4CC[C@H]4C(=O)N4CCN(CC(C)(F)F)CC4)nc(N)n3n2)o1] |
| 315 | 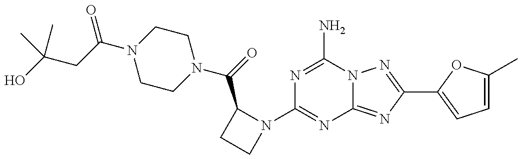 [SMILES: Cc1ccc(-c2nc3nc(N4CC[C@H]4C(=O)N4CCN(C(=O)CC(C)(C)O)CC4)nc(N)n3n2)o1] |
| 316 | 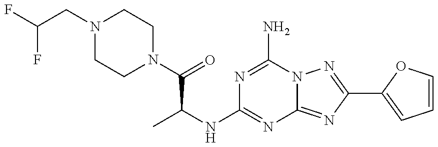 [SMILES: C[C@H](Nc1nc(N)n2nc(-c3ccco3)nc2n1)C(=O)N1CCN(CC(F)F)CC1] |
| 317 | 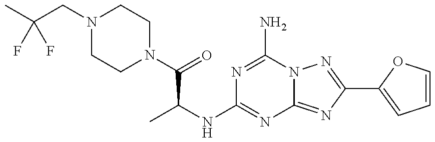 [SMILES: C[C@H](Nc1nc(N)n2nc(-c3ccco3)nc2n1)C(=O)N1CCN(CC(C)(F)F)CC1] |
| 318 | 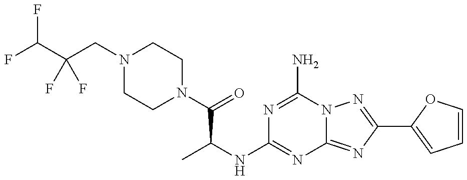 [SMILES: C[C@H](Nc1nc(N)n2nc(-c3ccco3)nc2n1)C(=O)N1CCN(CC(F)(F)C(F)F)CC1] |
| 319 | 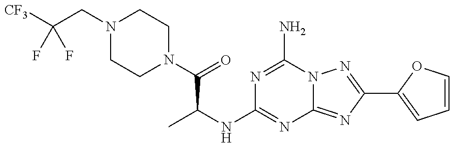 [SMILES: C[C@H](Nc1nc(N)n2nc(-c3ccco3)nc2n1)C(=O)N1CCN(CC(F)(F)C(F)(F)F)CC1] |
| 320 | 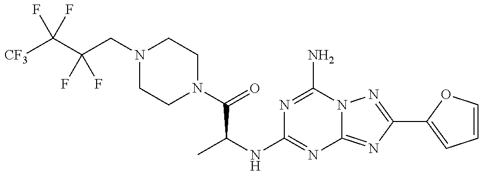 [SMILES: C[C@H](Nc1nc(N)n2nc(-c3ccco3)nc2n1)C(=O)N1CCN(CC(F)(F)C(F)(F)C(F)(F)F)CC1] |
| 321 | 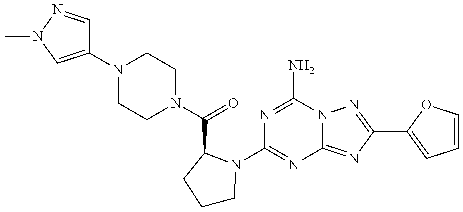 [SMILES: Cn1cc(N2CCN(C(=O)[C@@H]3CCCN3c3nc(N)n4nc(-c5ccco5)nc4n3)CC2)cn1] |
| 322 | 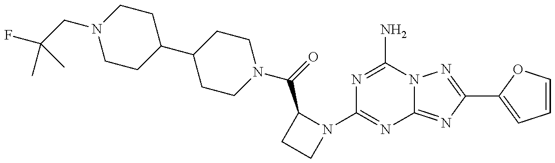 [SMILES: CC(C)(F)CN1CCC(C2CCN(C(=O)[C@@H]3CCN3c3nc(N)n4nc(-c5ccco5)nc4n3)CC2)CC1] |
| 323 | 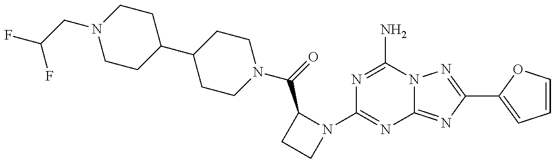 [SMILES: Nc1nc(N2CC[C@H]2C(=O)N2CCC(C3CCN(CC(F)F)CC3)CC2)nc2nc(-c3ccco3)nn12] |
| 324 | 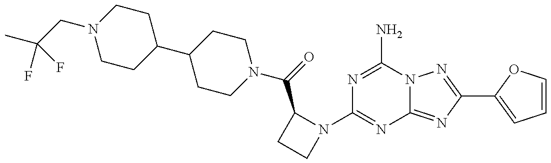 [SMILES: CC(F)(F)CN1CCC(C2CCN(C(=O)[C@@H]3CCN3c3nc(N)n4nc(-c5ccco5)nc4n3)CC2)CC1] |
| 325 | 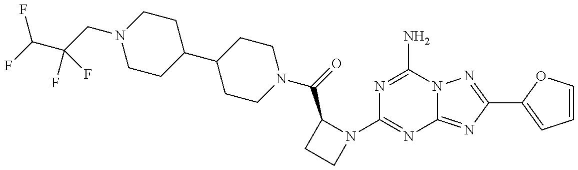 [SMILES: Nc1nc(N2CC[C@H]2C(=O)N2CCC(C3CCN(CC(F)(F)C(F)F)CC3)CC2)nc2nc(-c3ccco3)nn12] |
| 326 | 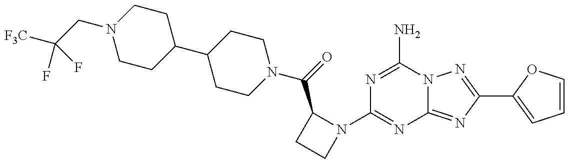 [SMILES: Nc1nc(N2CC[C@H]2C(=O)N2CCC(C3CCN(CC(F)(F)C(F)(F)F)CC3)CC2)nc2nc(-c3ccco3)nn12] |
| 327 | 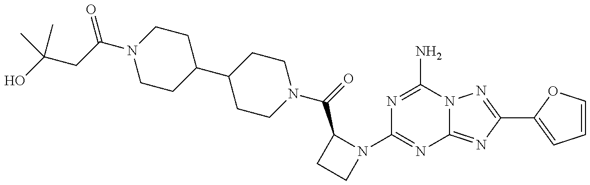 [SMILES: CC(C)(O)CC(=O)N1CCC(C2CCN(C(=O)[C@@H]3CCN3c3nc(N)n4nc(-c5ccco5)nc4n3)CC2)CC1] |
| 328 | 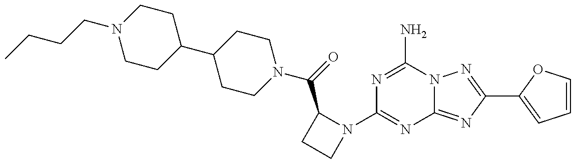 [SMILES: CCCCN1CCC(C2CCN(C(=O)[C@@H]3CCN3c3nc(N)n4nc(-c5ccco5)nc4n3)CC2)CC1] |
| 329 | 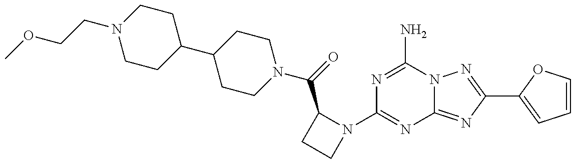 [SMILES: COCCN1CCC(C2CCN(C(=O)[C@@H]3CCN3c3nc(N)n4nc(-c5ccco5)nc4n3)CC2)CC1] |
| 330 | 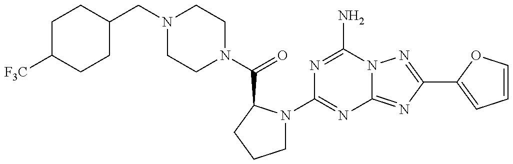 [SMILES: Nc1nc(N2CCC[C@H]2C(=O)N2CCN(CC3CCC(C(F)(F)F)CC3)CC2)nc2nc(-c3ccco3)nn12] |
| 331 | 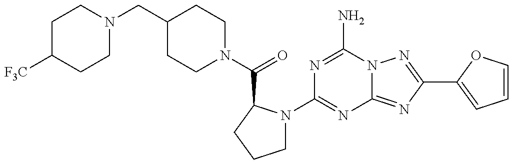 [SMILES: Nc1nc(N2CCC[C@H]2C(=O)N2CCC(CN3CCC(C(F)(F)F)CC3)CC2)nc2nc(-c3ccco3)nn12] |
| 332 | 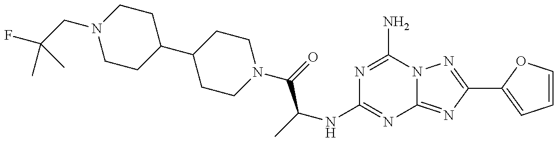 [SMILES: C[C@H](Nc1nc(N)n2nc(-c3ccco3)nc2n1)C(=O)N1CCC(C2CCN(CC(C)(C)F)CC2)CC1] |
| 333 | 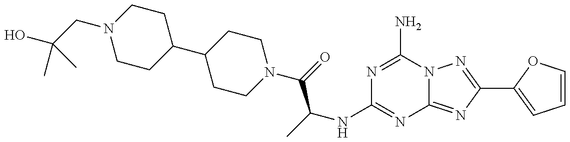 [SMILES: C[C@H](Nc1nc(N)n2nc(-c3ccco3)nc2n1)C(=O)N1CCC(C2CCN(CC(C)(C)O)CC2)CC1] |
| 334 | 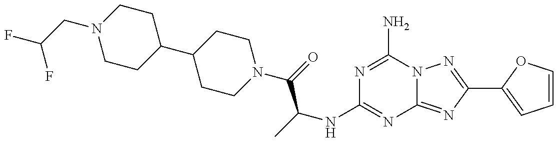 [SMILES: C[C@H](Nc1nc(N)n2nc(-c3ccco3)nc2n1)C(=O)N1CCC(C2CCN(CC(F)F)CC2)CC1] |
| 335 | 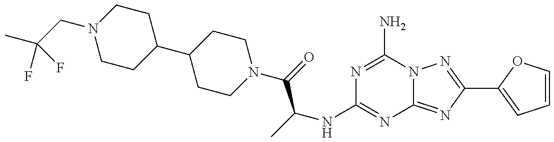 [SMILES: C[C@H](Nc1nc(N)n2nc(-c3ccco3)nc2n1)C(=O)N1CCC(C2CCN(CC(C)(F)F)CC2)CC1] |
| 336 | 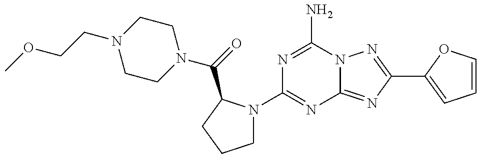 [SMILES: COCCN1CCN(C(=O)[C@@H]2CCCN2c2nc(N)n3nc(-c4ccco4)nc3n2)CC1] |
| 337 | 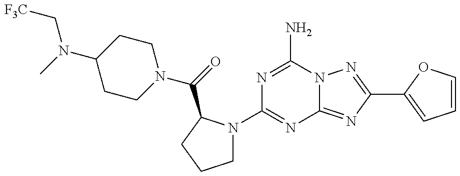 [SMILES: CN(CC(F)(F)F)C1CCN(C(=O)[C@@H]2CCCN2c2nc(N)n3nc(-c4ccco4)nc3n2)CC1] |
| 338 | 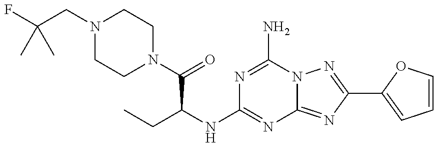 [SMILES: CC[C@H](Nc1nc(N)n2nc(-c3ccco3)nc2n1)C(=O)N1CCN(CC(C)(C)F)CC1] |
| 339 | 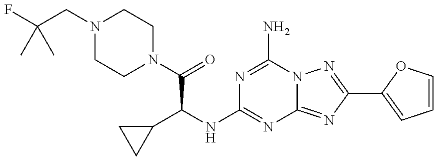 [SMILES: CC(C)(F)CN1CCN(C(=O)[C@@H](Nc2nc(N)n3nc(-c4ccco4)nc3n2)C2CC2)CC1] |
| 340 | 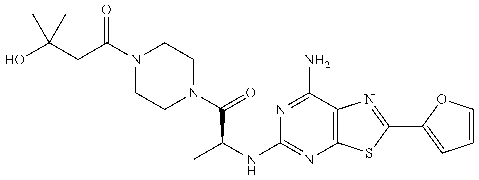 [SMILES: C[C@H](Nc1nc(N)c2nc(-c3ccco3)sc2n1)C(=O)N1CCN(C(=O)CC(C)(C)O)CC1] |
| 341 | 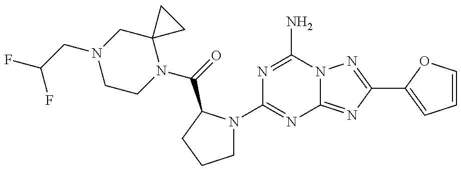 [SMILES: Nc1nc(N2CCC[C@H]2C(=O)N2CCN(CC(F)F)CC23CC3)nc2nc(-c3ccco3)nn12] |
| 342 | 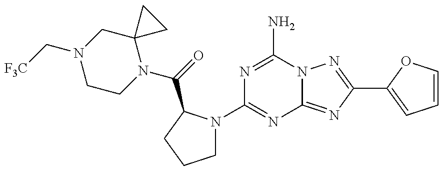 [SMILES: Nc1nc(N2CCC[C@H]2C(=O)N2CCN(CC(F)(F)F)CC23CC3)nc2nc(-c3ccco3)nn12] |
| 343 | 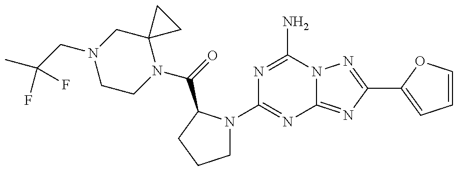 [SMILES: CC(F)(F)CN1CCN(C(=O)[C@@H]2CCCN2c2nc(N)n3nc(-c4ccco4)nc3n2)C2(CC2)C1] |
| 344 | 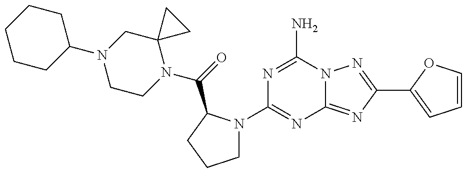 [SMILES: Nc1nc(N2CCC[C@H]2C(=O)N2CCN(C3CCCCC3)CC23CC3)nc2nc(-c3ccco3)nn12] |
| 345 | 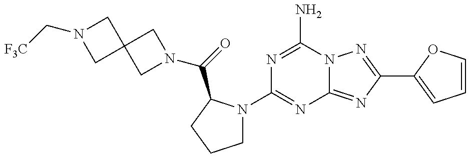 [SMILES: Nc1nc(N2CCC[C@H]2C(=O)N2CC3(CN(CC(F)(F)F)C3)C2)nc2nc(-c3ccco3)nn12] |
| 346 | 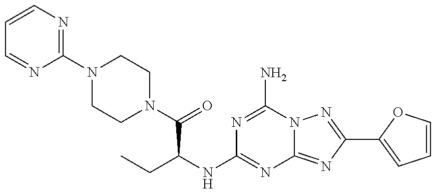 [SMILES: CC[C@H](Nc1nc(N)n2nc(-c3ccco3)nc2n1)C(=O)N1CCN(c2ncccn2)CC1] |
| 347 | 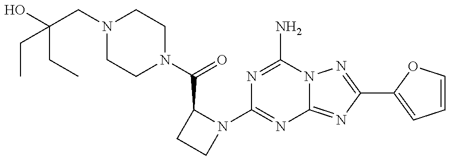 [SMILES: CCC(O)(CC)CN1CCN(C(=O)[C@@H]2CCN2c2nc(N)n3nc(-c4ccco4)nc3n2)CC1] |
| 348 | 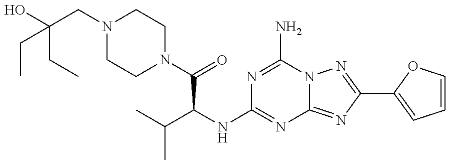 [SMILES: CCC(O)(CC)CN1CCN(C(=O)[C@@H](Nc2nc(N)n3nc(-c4ccco4)nc3n2)C(C)C)CC1] |
| 349 | 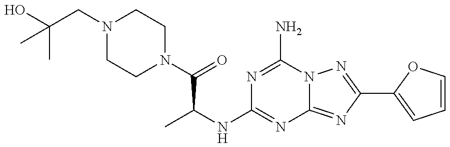 [SMILES: C[C@H](Nc1nc(N)n2nc(-c3ccco3)nc2n1)C(=O)N1CCN(CC(C)(C)O)CC1] |
| 350 | 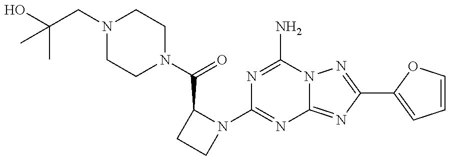 [SMILES: CC(C)(O)CN1CCN(C(=O)[C@@H]2CCN2c2nc(N)n3nc(-c4ccco4)nc3n2)CC1] |
| 351 | 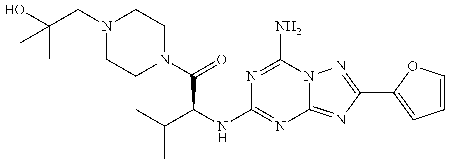 [SMILES: CC(C)[C@H](Nc1nc(N)n2nc(-c3ccco3)nc2n1)C(=O)N1CCN(CC(C)(C)O)CC1] |
| 352 | 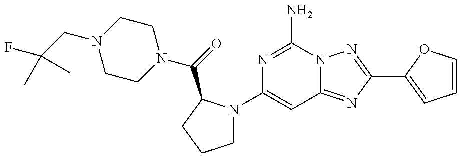 [SMILES: CC(C)(F)CN1CCN(C(=O)[C@@H]2CCCN2c2cc3nc(-c4ccco4)nn3c(N)n2)CC1] |
| 353 | 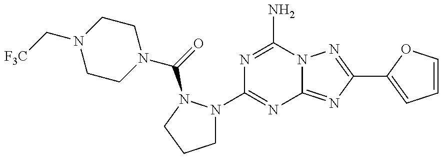 |
| 354 | 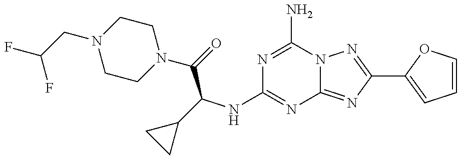 [SMILES: Nc1nc(N[C@H](C(=O)N2CCN(CC(F)F)CC2)C2CC2)nc2nc(-c3ccco3)nn12] |
| 355 | 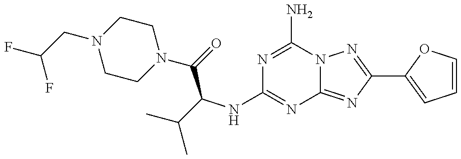 [SMILES: CC(C)[C@H](Nc1nc(N)n2nc(-c3ccco3)nc2n1)C(=O)N1CCN(CC(F)F)CC1] |
| 356 | 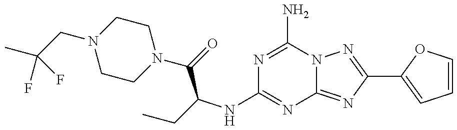 [SMILES: CC[C@H](Nc1nc(N)n2nc(-c3ccco3)nc2n1)C(=O)N1CCN(CC(C)(F)F)CC1] |
| 357 | 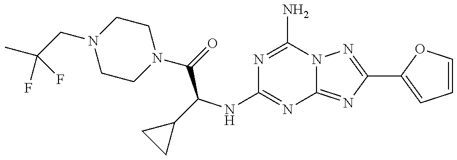 [SMILES: CC(F)(F)CN1CCN(C(=O)[C@@H](Nc2nc(N)n3nc(-c4ccco4)nc3n2)C2CC2)CC1] |
| 358 | 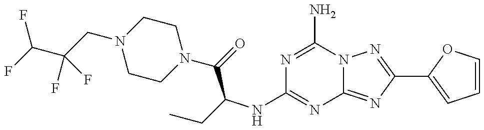 [SMILES: CC[C@H](Nc1nc(N)n2nc(-c3ccco3)nc2n1)C(=O)N1CCN(CC(F)(F)C(F)F)CC1] |
| 359 | 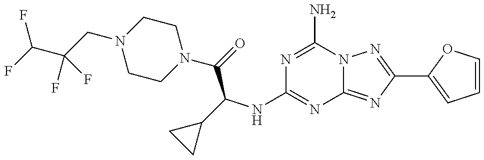 [SMILES: Nc1nc(N[C@H](C(=O)N2CCN(CC(F)(F)C(F)F)CC2)C2CC2)nc2nc(-c3ccco3)nn12] |
| 360 | 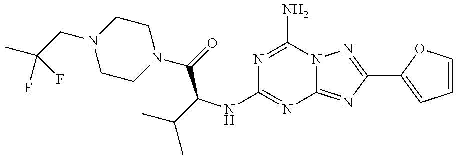 [SMILES: CC(C)[C@H](Nc1nc(N)n2nc(-c3ccco3)nc2n1)C(=O)N1CCN(CC(C)(F)F)CC1] |
| 361 | 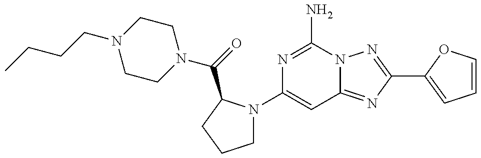 [SMILES: CCCCN1CCN(C(=O)[C@@H]2CCCN2c2cc3nc(-c4ccco4)nn3c(N)n2)CC1] |
| 362 | 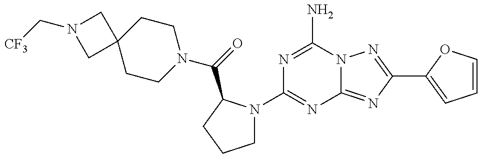 [SMILES: Nc1nc(N2CCC[C@H]2C(=O)N2CCC3(CC2)CN(CC(F)(F)F)C3)nc2nc(-c3ccco3)nn12] |
| 363 | 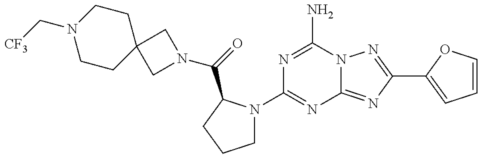 [SMILES: Nc1nc(N2CCC[C@H]2C(=O)N2CC3(CCN(CC(F)(F)F)CC3)C2)nc2nc(-c3ccco3)nn12] |
| 364 | 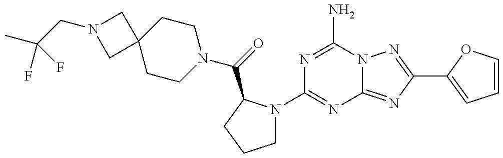 [SMILES: CC(F)(F)CN1CC2(CCN(C(=O)[C@@H]3CCCN3c3nc(N)n4nc(-c5ccco5)nc4n3)CC2)C1] |
| 365 | 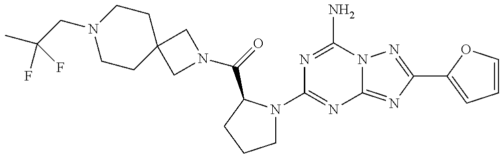 [SMILES: CC(F)(F)CN1CCC2(CC1)CN(C(=O)[C@@H]1CCCN1c1nc(N)n3nc(-c4ccco4)nc3n1)C2] |
| 366 | 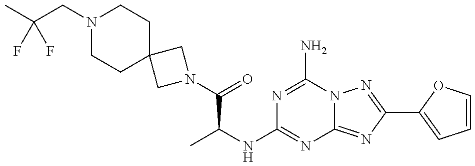 [SMILES: C[C@H](Nc1nc(N)n2nc(-c3ccco3)nc2n1)C(=O)N1CC2(CCN(CC(C)(F)F)CC2)C1] |
| 367 | 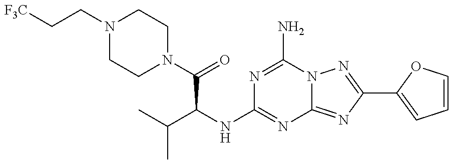 [SMILES: CC(C)[C@H](Nc1nc(N)n2nc(-c3ccco3)nc2n1)C(=O)N1CCN(CCC(F)(F)F)CC1] |
| 368 | 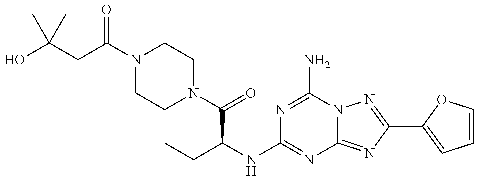 [SMILES: CC[C@H](Nc1nc(N)n2nc(-c3ccco3)nc2n1)C(=O)N1CCN(C(=O)CC(C)(C)O)CC1] |
| 369 | 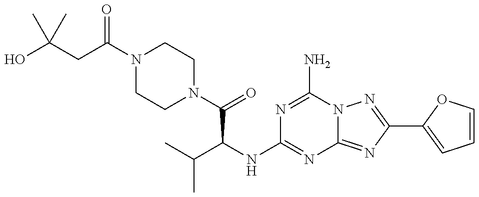 [SMILES: CC(C)[C@H](Nc1nc(N)n2nc(-c3ccco3)nc2n1)C(=O)N1CCN(C(=O)CC(C)(C)O)CC1] |
| 370 | 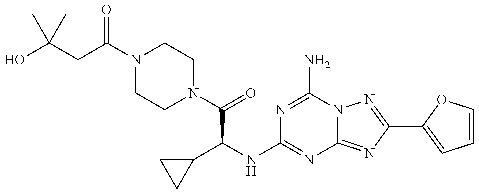 [SMILES: CC(C)(O)CC(=O)N1CCN(C(=O)[C@@H](Nc2nc(N)n3nc(-c4ccco4)nc3n2)C2CC2)CC1] |
| 371 | 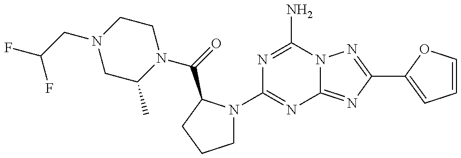 [SMILES: C[C@@H]1CN(CC(F)F)CCN1C(=O)[C@@H]1CCCN1c1nc(N)n2nc(-c3ccco3)nc2n1] |
| 372 | 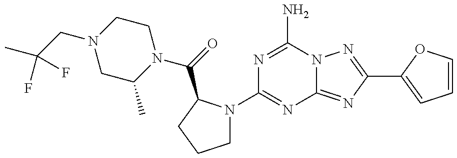 [SMILES: C[C@@H]1CN(CC(C)(F)F)CCN1C(=O)[C@@H]1CCCN1c1nc(N)n2nc(-c3ccco3)nc2n1] |
| 373 | 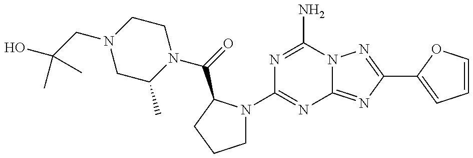 [SMILES: C[C@@H]1CN(CC(C)(C)O)CCN1C(=O)[C@@H]1CCCN1c1nc(N)n2nc(-c3ccco3)nc2n1] |
| 374 | 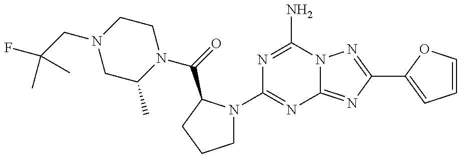 [SMILES: C[C@@H]1CN(CC(C)(C)F)CCN1C(=O)[C@@H]1CCCN1c1nc(N)n2nc(-c3ccco3)nc2n1] |
| 375 | 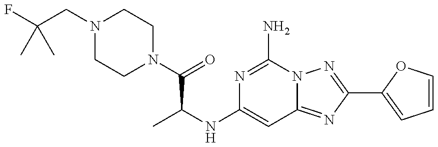 [SMILES: C[C@H](Nc1cc2nc(-c3ccco3)nn2c(N)n1)C(=O)N1CCN(CC(C)(C)F)CC1] |
| 376 | 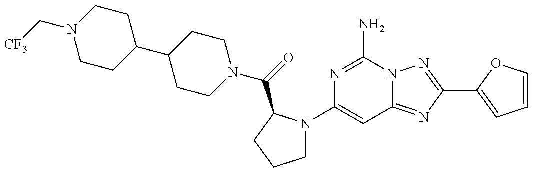 [SMILES: Nc1nc(N2CCC[C@H]2C(=O)N2CCC(C3CCN(CC(F)(F)F)CC3)CC2)cc2nc(-c3ccco3)nn12] |
| 377 | 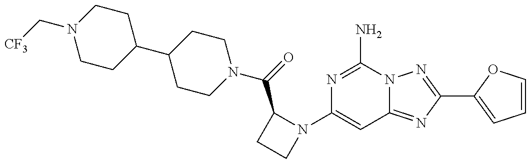 [SMILES: Nc1nc(N2CC[C@H]2C(=O)N2CCC(C3CCN(CC(F)(F)F)CC3)CC2)cc2nc(-c3ccco3)nn12] |
| 378 | 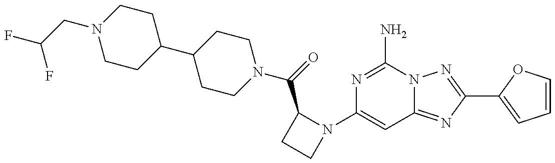 [SMILES: Nc1nc(N2CC[C@H]2C(=O)N2CCC(C3CCN(CC(F)F)CC3)CC2)cc2nc(-c3ccco3)nn12] |
| 379 | 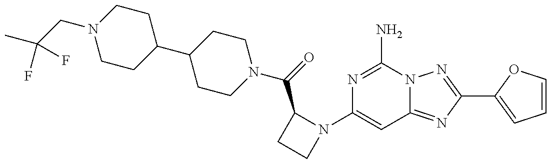 [SMILES: CC(F)(F)CN1CCC(C2CCN(C(=O)[C@@H]3CCN3c3cc4nc(-c5ccco5)nn4c(N)n3)CC2)CC1] |
| 380 | 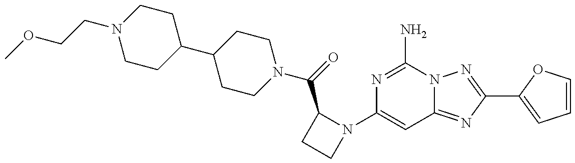 [SMILES: COCCN1CCC(C2CCN(C(=O)[C@@H]3CCN3c3cc4nc(-c5ccco5)nn4c(N)n3)CC2)CC1] |
| 381 | 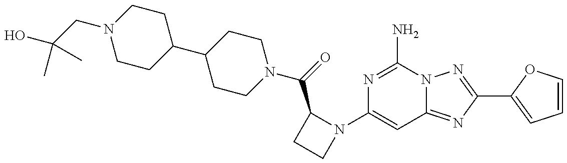 [SMILES: CC(C)(O)CN1CCC(C2CCN(C(=O)[C@@H]3CCN3c3cc4nc(-c5ccco5)nn4c(N)n3)CC2)CC1] |
| 382 | 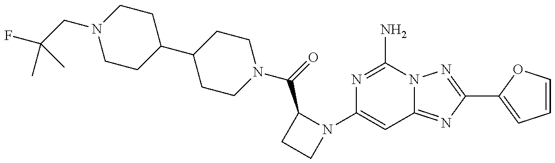 [SMILES: CC(C)(F)CN1CCC(C2CCN(C(=O)[C@@H]3CCN3c3cc4nc(-c5ccco5)nn4c(N)n3)CC2)CC1] |
| 383 | 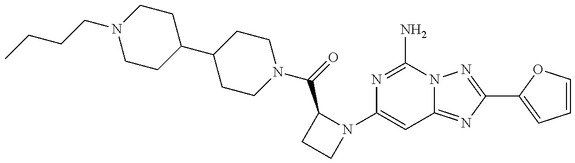 [SMILES: CCCCN1CCC(C2CCN(C(=O)[C@@H]3CCN3c3cc4nc(-c5ccco5)nn4c(N)n3)CC2)CC1] |
| 384 | 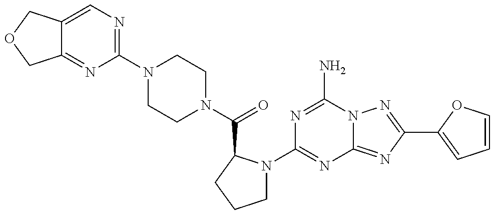 [SMILES: Nc1nc(N2CCC[C@H]2C(=O)N2CCN(c3ncc4c(n3)COC4)CC2)nc2nc(-c3ccco3)nn12] |
| 385 | 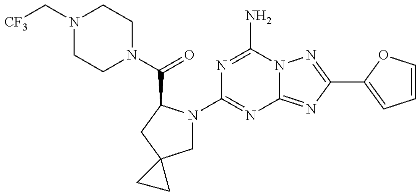 [SMILES: Nc1nc(N2CC3(CC3)C[C@H]2C(=O)N2CCN(CC(F)(F)F)CC2)nc2nc(-c3ccco3)nn12] |
| 386 | 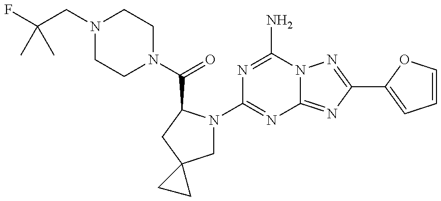 [SMILES: CC(C)(F)CN1CCN(C(=O)[C@@H]2CC3(CC3)CN2c2nc(N)n3nc(-c4ccco4)nc3n2)CC1] |
| 387 | 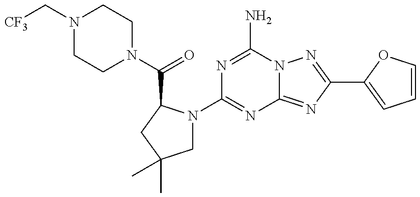 [SMILES: CC1(C)C[C@@H](C(=O)N2CCN(CC(F)(F)F)CC2)N(c2nc(N)n3nc(-c4ccco4)nc3n2)C1] |
| 388 | 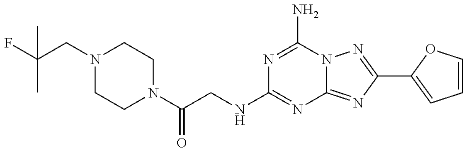 [SMILES: CC(C)(F)CN1CCN(C(=O)CNc2nc(N)n3nc(-c4ccco4)nc3n2)CC1] |
| 389 | 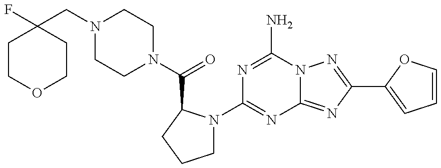 [SMILES: Nc1nc(N2CCC[C@H]2C(=O)N2CCN(CC3(F)CCOCC3)CC2)nc2nc(-c3ccco3)nn12] |
| 390 | 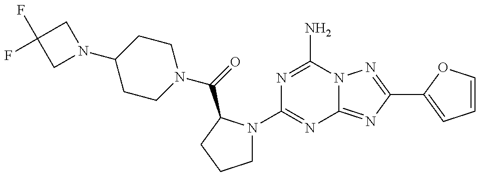 [SMILES: Nc1nc(N2CCC[C@H]2C(=O)N2CCC(N3CC(F)(F)C3)CC2)nc2nc(-c3ccco3)nn12] |
| 391 | 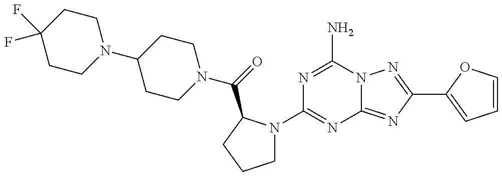 [SMILES: Nc1nc(N2CCC[C@H]2C(=O)N2CCC(N3CCC(F)(F)CC3)CC2)nc2nc(-c3ccco3)nn12] |
| 392 | 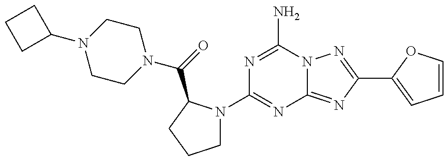 [SMILES: Nc1nc(N2CCC[C@H]2C(=O)N2CCN(C3CCC3)CC2)nc2nc(-c3ccco3)nn12] |
| 393 | 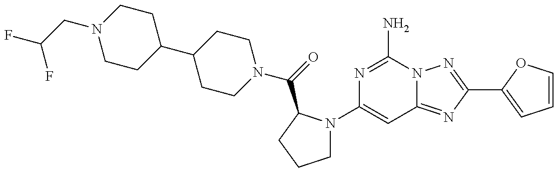 [SMILES: Nc1nc(N2CCC[C@H]2C(=O)N2CCC(C3CCN(CC(F)F)CC3)CC2)cc2nc(-c3ccco3)nn12] |
| 394 | 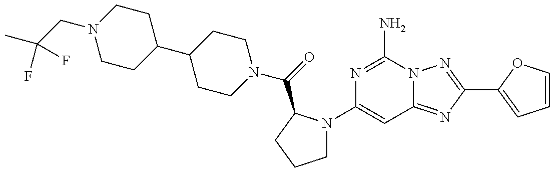 [SMILES: CC(F)(F)CN1CCC(C2CCN(C(=O)[C@@H]3CCCN3c3cc4nc(-c5ccco5)nn4c(N)n3)CC2)CC1] |
| 395 | 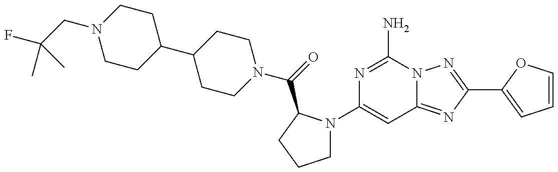 [SMILES: CC(C)(F)CN1CCC(C2CCN(C(=O)[C@@H]3CCCN3c3cc4nc(-c5ccco5)nn4c(N)n3)CC2)CC1] |
| 396 | 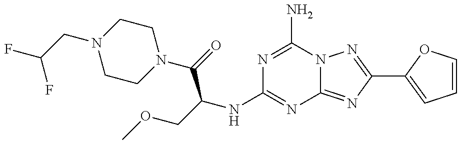 [SMILES: COC[C@H](Nc1nc(N)n2nc(-c3ccco3)nc2n1)C(=O)N1CCN(CC(F)F)CC1] |
| 397 | 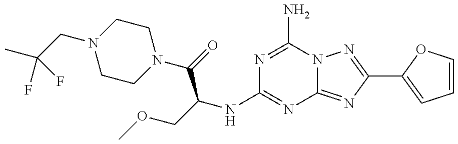 [SMILES: COC[C@H](Nc1nc(N)n2nc(-c3ccco3)nc2n1)C(=O)N1CCN(CC(C)(F)F)CC1] |
| 398 | 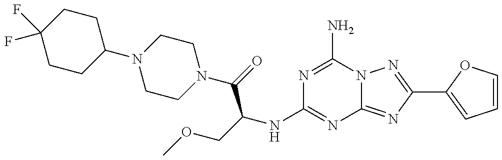 [SMILES: COC[C@H](Nc1nc(N)n2nc(-c3ccco3)nc2n1)C(=O)N1CCN(C2CCC(F)(F)CC2)CC1] |
| 399 | 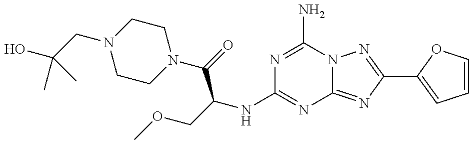 [SMILES: COC[C@H](Nc1nc(N)n2nc(-c3ccco3)nc2n1)C(=O)N1CCN(CC(C)(C)O)CC1] |
| 400 | 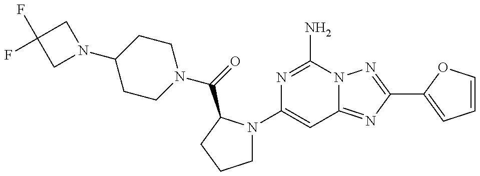 [SMILES: Nc1nc(N2CCC[C@H]2C(=O)N2CCC(N3CC(F)(F)C3)CC2)cc2nc(-c3ccco3)nn12] |
| 401 | 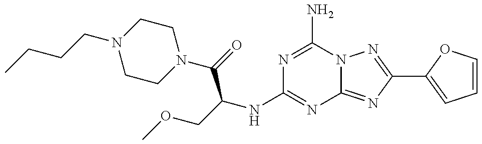 [SMILES: CCCCN1CCN(C(=O)[C@H](COC)Nc2nc(N)n3nc(-c4ccco4)nc3n2)CC1] |
| 402 | 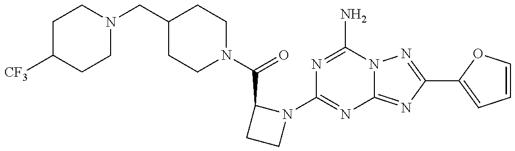 [SMILES: Nc1nc(N2CC[C@H]2C(=O)N2CCC(CN3CCC(C(F)(F)F)CC3)CC2)nc2nc(-c3ccco3)nn12] |
| 403 | 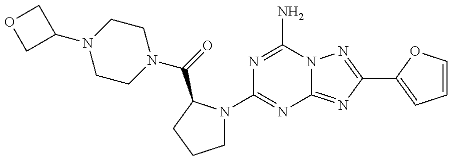 [SMILES: Nc1nc(N2CCC[C@H]2C(=O)N2CCN(C3COC3)CC2)nc2nc(-c3ccco3)nn12] |
| 404 | 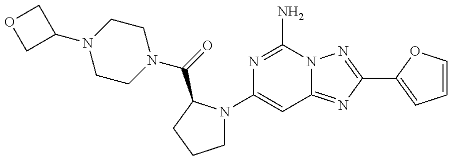 [SMILES: Nc1nc(N2CCC[C@H]2C(=O)N2CCN(C3COC3)CC2)cc2nc(-c3ccco3)nn12] |
| 405 | 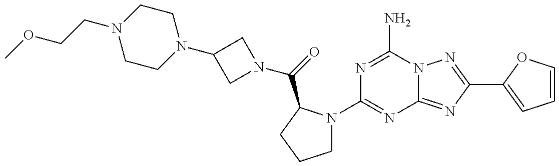 [SMILES: COCCN1CCN(C2CN(C(=O)[C@@H]3CCCN3c3nc(N)n4nc(-c5ccco5)nc4n3)C2)CC1] |
| 406 | 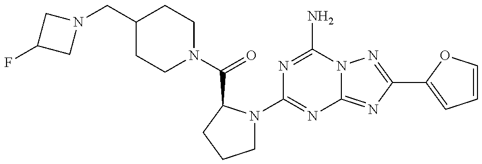 [SMILES: Nc1nc(N2CCC[C@H]2C(=O)N2CCC(CN3CC(F)C3)CC2)nc2nc(-c3ccco3)nn12] |

6. The compound, stereoisomers thereof or pharmaceutically acceptable salts thereof according to claim 5, wherein the compound is any one selected from the group consisting of compounds below:

| Example | |
|---|---|
| No. | Compound Structure |
| 25 | 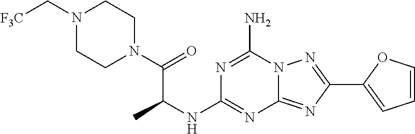 [SMILES: C[C@H](Nc1nc(N)n2nc(-c3ccco3)nc2n1)C(=O)N1CCN(CC(F)(F)F)CC1] |
| 26 | 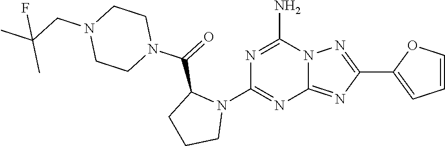 [SMILES: CC(C)(F)CN1CCN(C(=O)[C@@H]2CCCN2c2nc(N)n3nc(-c4ccco4)nc3n2)CC1] |
| 48 | 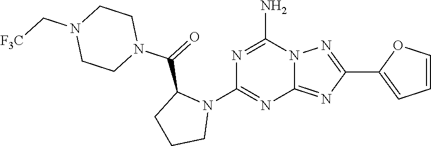 [SMILES: Nc1nc(N2CCC[C@H]2C(=O)N2CCN(CC(F)(F)F)CC2)nc2nc(-c3ccco3)nn12] |
| 90 | 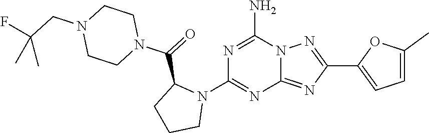 [SMILES: Cc1ccc(-c2nc3nc(N4CCC[C@H]4C(=O)N4CCN(CC(C)(C)F)CC4)nc(N)n3n2)o1] |
| 111 | 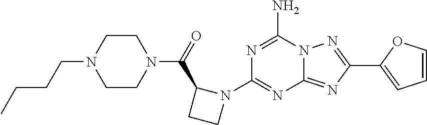 [SMILES: CCCCN1CCN(C(=O)[C@@H]2CCN2c2nc(N)n3nc(-c4ccco4)nc3n2)CC1] |
| 223 | 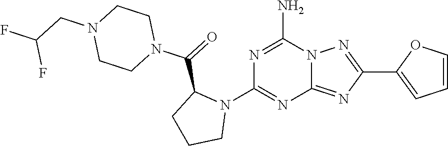 [SMILES: Nc1nc(N2CCC[C@H]2C(=O)N2CCN(CC(F)F)CC2)nc2nc(-c3ccco3)nn12] |
| 224 | 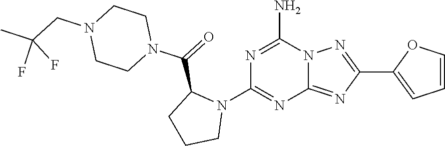 [SMILES: CC(F)(F)CN1CCN(C(=O)[C@@H]2CCCN2c2nc(N)n3nc(-c4ccco4)nc3n2)CC1] |
| 294 | 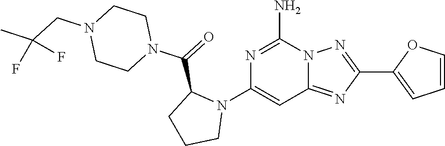 [SMILES: CC(F)(F)CN1CCN(C(=O)[C@@H]2CCCN2c2cc3nc(-c4ccco4)nn3c(N)n2)CC1] |
| 303 | 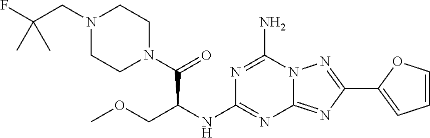 [SMILES: COC[C@H](Nc1nc(N)n2nc(-c3ccco3)nc2n1)C(=O)N1CCN(CC(C)(C)F)CC1] |
| 353 | 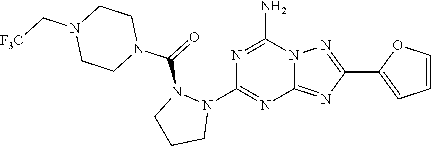 |
| 371 | 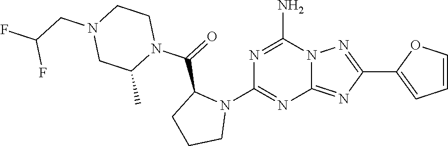 [SMILES: C[C@@H]1CN(CC(F)F)CCN1C(=O)[C@@H]1CCCN1c1nc(N)n2nc(-c3ccco3)nc2n1] |

7. A pharmaceutical composition comprising the compound according to claim 1, stereoisomers thereof or pharmaceutically acceptable salts thereof as an active ingredient.

8. The pharmaceutical composition according to claim 7, wherein the pharmaceutical composition is for treating or preventing adenosine A2a receptor-associated diseases.

9. The pharmaceutical composition according to claim 8, wherein the adenosine A2a receptor-associated diseases are cancer or inflammatory diseases.

10. The pharmaceutical composition according to claim 9, wherein the cancer is at least one selected from lung cancer, stomach cancer, ovarian cancer, prostate cancer, esophageal cancer, gastrointestinal cancer, pancreatic cancer, colorectal cancer, kidney cancer, testicular cancer, bladder cancer, breast cancer, uterine cancer, cervical cancer, head and neck cancer, blood cancer, bone cancer, liver cancer, thyroid cancer, skin cancer, lymphoma, leukemia, myeloma, sarcoma and virus-associated cancer.

11. The pharmaceutical composition according to claim 9, wherein the inflammatory disease is at least one selected from rheumatoid arthritis, multiple sclerosis, Crohn's disease, ulcerative colitis, graft-versus-host disease, systemic lupus erythematosus, toxic shock syndrome, osteoarthritis, and insulin-dependent diabetes.

12. A method for treating or preventing adenosine A2a receptor-associated diseases, the method administering an effective amount of the compound according to claim 1, stereoisomers thereof or pharmaceutically acceptable salts thereof.

13. (canceled)

14. (canceled)

15. A pharmaceutical composition comprising the compound according to claim 5, stereoisomers thereof or pharmaceutically acceptable salts thereof as an active ingredient.

16. The pharmaceutical composition according to claim 15, wherein the pharmaceutical composition is for treating or preventing adenosine A2a receptor-associated diseases.

17. The pharmaceutical composition according to claim 16, wherein the adenosine A2a receptor-associated diseases are cancer or inflammatory diseases.

18. The pharmaceutical composition according to claim 17, wherein the cancer is at least one selected from lung cancer, stomach cancer, ovarian cancer, prostate cancer, esophageal cancer, gastrointestinal cancer, pancreatic cancer, colorectal cancer, kidney cancer, testicular cancer, bladder cancer, breast cancer, uterine cancer, cervical cancer, head and neck cancer, blood cancer, bone cancer, liver cancer, thyroid cancer, skin cancer, lymphoma, leukemia, myeloma, sarcoma and virus-associated cancer.

19. The pharmaceutical composition according to claim 17, wherein the inflammatory disease is at least one selected from rheumatoid arthritis, multiple sclerosis, Crohn's disease, ulcerative colitis, graft-versus-host disease, systemic lupus erythematosus, toxic shock syndrome, osteoarthritis, and insulin-dependent diabetes.

20. A method for treating or preventing adenosine A2a receptor-associated diseases, the method administering an effective amount of the compound according to claim 5, stereoisomers thereof or pharmaceutically acceptable salts thereof.
